(19) United States
(12) Patent Application Publication
(10) Pub. No.: US 2024/0075097 A1
(43) Pub. Date: Mar. 7, 2024

(54) THERAPEUTIC PEPTIDES

(71) Applicants: UNIVERSITE TOULOUSE III-PAUL SABATIER, Toulouse Cedex 9 (FR); CENTRE NATIONAL DE LA RECHERCHE SCIENTIFIQUE, Paris Cedex 16 (FR); INSERM (INSTITUT NATIONAL DE LA SANTE ET DE LA RECHERCHE MEDICALE), Paris Cedex 13 (FR); ETABLISSEMENT FRANÇAIS DU SANG, La Plaine Saint Denis (FR)

(72) Inventors: Jean-Philippe COMBIER, Castanet Tolosan (FR); Anne PREL, Toulouse (FR); Frédéric DESCHASEAUX, Toulouse (FR)

(73) Assignees: UNIVERSITE TOULOUSE III-PAUL SABATIER, Toulouse Cedex 9 (FR); CENTRE NATIONAL DE LA RECHERCHE SCIENTIFIQUE, Paris Cedex 16 (FR); INSERM (INSTITUT NATIONAL DE LA SANTE ET DE LA RECHERCHE MEDICALE), Paris Cedex 13 (FR); ETABLISSEMENT FRANÇAIS DU SANG, La Plaine Saint Denis (FR)

(21) Appl. No.: 18/146,779

(22) Filed: Dec. 27, 2022

Related U.S. Application Data
(62) Division of application No. 16/481,111, filed on Jul. 26, 2019, now Pat. No. 11,607,443.

(30) Foreign Application Priority Data
Dec. 22, 2016 (FR) 16/63245

(51) Int. Cl. A61K 38/17; A61P 9/10; A61P 11/00; A61P 19/08; A61P 31/14; A61P 35/00; C12Q 1/6883

(52) U.S. Cl. CPC A61K 38/1709; A61P 9/10; A61P 11/00; A61P 19/08; A61P 31/14; A61P 35/00; C12Q 1/6883; C12Q 2600/178

(57) ABSTRACT

Compositions containing micropeptides capable of modulating the accumulation of miRs involved in certain pathologies, and the use of these micropeptides for treating the certain pathologies. Also, methods of identifying these micropeptides modulating the accumulation of miRs involved in pathologies. Further, nuclei acids encoding those micropeptides that modulate the accumulation of miRs involved in pathologies.

### FIELD

The present invention relates to therapeutic peptides.

### REFERENCE TO A SEQUENCE LISTING

In accordance with 37 CFR § 1.831, the present specification makes reference to a Sequence Listing submitted electronically as an “.xml” file named “USD PHAR2—Seq. Listing ST.26.xml”. The .xml file was generated on Jul. 19, 2023 and is 11,055,713 bytes in size. The entire contents of the Sequence Listing are hereby incorporated by reference.

### BACKGROUND

The microRNAs (miRs) are small non-coding RNAs, about 21 nucleotides in length after maturation, which control expression of target genes at the post-transcriptional level, by degrading the target mRNA or by inhibiting its translation, in eukaryotic organisms. The miRs can in particular regulate the expression of specific genes involved in certain pathologies in humans and animals. It is now recognized that miRs play an important role in many pathologies, and these are therefore attractive targets for the development of new drugs. The regulation of expression of the miRs is very poorly understood, but it is known in particular that the latter involves, like most coding genes, an RNA polymerase II: this enzyme produces a primary transcript, called “pri-miR”, which is then matured by a protein complex in particular containing the Dicer type enzymes. This maturation leads firstly to the formation of a precursor of miR called “pre-miR”, having a stem-loop secondary structure containing the miR and its complementary sequence miR*. Then the precursor is matured, which leads to formation of a shorter double-stranded RNA containing the miRNA and the miR*. The miR is then manipulated by the RISC complex, which cleaves the mRNA of the target gene or inhibits its translation.

At present, there are mainly two types of therapeutic approaches for regulating the expression of miRs in vivo.

The first approach is to mimic the effect of miR using synthetic RNA duplexes designed to mimic the miR of interest. One strand is identical to the miR of interest, while the other strand can be modified to facilitate cellular uptake of the molecule. However, since one of the strands must function as a miR and be recognized as such by the cell, the allowed modifications are chemically limited. In addition, although this approach allows for the replacement of the amounts of miRs lost during a pathology, it is difficult to target the RNA molecules to specific tissues, and the tissues that do not normally express the miR of interest. can also capture the synthetic RNA duplex and lead to undesirable side effects.

The second approach is to use antisense oligonucleotides. Such antisense oligonucleotides have a sequence complementary to all or part of that of the target miR and will reduce the endogenous amount of miRs. However, because of the low in vivo stability of this type of molecule, their manipulation is made difficult and expensive.

It is therefore necessary to provide a means of regulating more simply, and specifically, the miRs involved in certain pathologies.

Recently, small open reading frames (ORFs) have also been found in long intergenic non-coding RNAs (lincRNAs) whose putative function, if any, is not known (Ingolia et al., Cell, 147(4):789-802, 2011; Guttman & Rinn, Nature, 482(7385):339-46, 2012). However, no examples have yet been reported concerning the existence of ORFs encoding peptides within miRs. So far, the miRs, and by extension their primary transcript, have always been considered, by their particular mode of action, as noncoding regulatory RNAs producing no peptide.

However, the inventors have recently demonstrated the existence of micropeptides (or “miPEPs”, microRNA encoded PEPtides) capable of modulating the accumulation of miRs in cells (Lauressergues et al., Nature, 520(7545):90-3, 2015; International Application WO 2015/063431).

### SUMMARY

One of the aspects of the invention is therefore to propose compositions containing micropeptides capable of modulating the accumulation of miRs involved in certain pathologies. Another aspect of the invention relates to these micropeptides for their use as medicaments, and for the treatment of specific pathologies.

Another aspect of the invention relates to a method of identifying micropeptides for modulating the accumulation of miRs involved in pathologies.

Other aspects of the invention also relate to micropeptides as such and the nucleic acids encoding them.

### BRIEF DESCRIPTION OF THE DRAWINGS

FIG. 1: List indicating the corresponding miPEPs and miORPs, as well as the associated miRs.

FIG. 2: Expression of the iBSP gene in mesenchymal stem cells cultured in DIF+BMP4 medium in presence of miPEP//125a-1.

The mesenchymal stem cells were treated for 4 days with 100 μM of miPEP//125a-1, with 100 μM of control peptide (ScmiPEP//125a) or untreated (NT). The y-axis indicates the relative expression of the iBSP gene. Analyzes and statistics were performed with the Mann-Withney test (p-value: *p<0.05; **p<0.01 and ***p<0.001).

FIG. 3: Expression of the PTHR1 gene in mesenchymal stem cells cultured in DIF+BMP4 medium in presence of miPEP//125a-1.

The mesenchymal stem cells were treated for 4 days with 100 μM of miPEP//125a-1, with 100 μM of control peptide (ScmiPEP//125a) or untreated (NT). The y-axis indicates the relative expression of the PTHR1 gene. Analyzes and statistics were performed with the Mann-Withney test (p-value: *p<0.05; **p<0.01 and ***p<0.001).

FIG. 4: Expression of the STMN2 gene in mesenchymal stem cells cultured in DIF+BMP4 medium in presence of miPEP//125a-1.

The mesenchymal stem cells were treated for 4 days with 100 μM of miPEP//125a-1, with 100 μM of control peptide (ScmiPEP//125a) or untreated (NT). The y-axis indicates the relative expression of the STMN2 gene. Analyzes and statistics were performed with the Mann-Withney test (p-value: *p<0.05; **p<0.01 and ***p<0.001).

FIG. 5: Expression of the OSTERIX gene in mesenchymal stem cells cultured in DIF+BMP4 medium in presence of miPEP//125a-1.

The mesenchymal stem cells were treated for 4 days with 100 μM of miPEP//125a-1, with 100 μM of control peptide (ScmiPEP//125a) or untreated (NT). The y-axis indicates the relative expression of the OSTERIX gene. Analyzes and statistics were performed with the Mann-Withney test (p-value: *p<0.05; **p<0.01 and ***p<0.001).

FIG. 6: Expression of the STMN2 gene in mesenchymal stem cells cultured in DIF+BMP4 medium in presence of miPEP//145-2.

The mesenchymal stem cells were treated for 4 days with 100 μM of miPEP//145-2, with 100 μM of control peptide (ScmiPEP//125a) or untreated (NT). The y-axis indicates the relative expression of the STMN2 gene. Analyzes and statistics were performed with the Mann-Withney test (p-value: *p<0.05; **p<0.01 and ***p<0.001).

FIG. 7: Expression of the STMN2 gene in mesenchymal stem cells cultured in DIFΔBMP4 medium in presence of miPEP//125a-1.

The mesenchymal stem cells were treated for 4 days with 100 μM of miPEP//125a-1, with 100 μM of control peptide (ScmiPEP//125a) or untreated (NT). The y-axis indicates the relative expression of the STMN2 gene. Analyzes and statistics were performed with the Mann-Withney test (p-value: *p<0.05; **p<0.01 and ***p<0.001).

FIG. 8: Expression of the OSTERIX gene in mesenchymal stem cells cultured in DIF Δ BMP4 medium in presence of miPEP//125a-1.

The mesenchymal stem cells were treated for 4 days with 100 μM of miPEP//125a-1, with 100 μM of control peptide (ScmiPEP//125a) or untreated (NT). The y-axis indicates the relative expression of the OSTERIX gene. Analyzes and statistics were performed with the Mann-Withney test (p-value: *p<0.05; **p<0.01 and ***p<0.001).

FIG. 9: Expression of the STMN2 gene in mesenchymal stem cells cultured in DIF Δ BMP4 medium in presence of miPEP//145-2.

The mesenchymal stem cells were treated for 4 days with 100 μM of miPEP//145-2, with 100 μM of control peptide (ScmiPEP//125a) or untreated (NT). The y-axis indicates the relative expression of the STMN2 gene. Analyzes and statistics were performed with the Mann-Withney test (p-value: *p<0.05; **p<0.01 and ***p<0.001).

FIG. 10: Expression of the OSTERIX gene in mesenchymal stem cells cultured in DIF Δ BMP4 medium in presence of miPEP//145-2.

The mesenchymal stem cells were treated for 4 days with 100 μM of miPEP//145-2, with 100 μM of control peptide (ScmiPEP//125a) or untreated (NT). The y-axis indicates the relative expression of the OSTERIX gene. Analyzes and statistics were performed with the Mann-Withney test (p-value: *p<0.05; **p<0.01 and ***p<0.001).

FIG. 11: Expression of the STMN2 gene in mesenchymal stem cells cultured in DIF Δ BMP4 medium in presence of miPEP//145-2.

The mesenchymal stem cells were treated for 4 days with 100 μM of miPEP//145-2, with 100 μM of control peptide (ScmiPEP//125a) or untreated (NT). The y-axis indicates the relative expression of the STMN2 gene. Analyzes and statistics were performed with the Mann-Withney test (p-value: *p<0.05; **p<0.01 and ***p<0.001).

FIG. 12: Entry of miPEP//15a-16-FAM in mesenchymal stem cells. The undifferentiated mesenchymal stem cells were treated for 6 hours with miPEP//15a-16-1-1-FAM at 5 μM (A), 10 μM (B), 50 μM (C) or 100 μM (D).

FIG. 13: Entry of miPEP//15a-16-FAM in mesenchymal stem cells.

The undifferentiated mesenchymal stem cells were treated with 100 μM of miPEP//15a-16-1-1-FAM for 2 hours (A), 4 hours (B), 6 hours (C), 8 hours (D) and 24 hours (E).

FIG. 14: Entry of miPEP//15a-16-FAM-TAT in mesenchymal stem cells.

The undifferentiated mesenchymal stem cells were treated for 6 hours with miPEP//15a-16-1-1-FAM-TAT at 5 μM (A), 10 μM (B), 20 μM (C), 50 μM (D) or 100 μM (E).

FIG. 15: Entry of miPEP//15a-16-FAM-TAT in mesenchymal stem cells.

The undifferentiated mesenchymal stem cells were treated for 2 hours with miPEP//15a-16-1-1-FAM-TAT at 10 μM.

FIG. 16: Accumulation of the miR16-1 precursor in the presence of miPEP//15a-16-1-TAT. The mesenchymal stem cells were treated with 10 μM of miPEP//15a-16-1-TAT, treated with 10 μM of control peptide (ScmiPEP//21-1) or untreated (NT) for 3 hours. The amount of miR16-1 precursor in cells has been measured by quantitative RT-PCR. The measurement of the amount of miR16-1 precursor was normalized with respect to the PPIA household gene. Analyzes and statistics were performed with the Mann-Withney test (p-value: *p<0.05; **p<0.01 and ***p<0.001).

FIG. 17: Evaluation of the effects of miPEP125a-1 on bone formation in vivo.

Nude mice were grafted with
A. the biomaterial alone,
B. mesenchymal stem cells mixed with biomaterial (granules of βTCP [20/80]),
C. & D. mesenchymal stem cells pretreated for 4 days with miPEP//125a-1 mixed with biomaterial (granules of βTCP [20/80]),
E. & F. mesenchymal stem cells pre-treated 4 days with the control miPEP (scramble) mixed with biomaterial (granules of βTCP [20/80]).

For D. and F. conditions, mice also received regular injections (once a week) of either miPEP//125a-1 (D) or scramble miPEP (F) during the study. After 4 weeks, the mice are euthanized and the grafts were then isolated, fixed, included in methacrylate, cut and treated for histology. The cuts were scanned (NDPview) and bone formation (dark grey) is evaluated (A to F) and is also quantified (G) using the Fiji image analysis software and the total area of new bone is reported to the total area of the cut

With regard to microscopic photographs A to F, the scale is indicated in the figure. For G, the y-axis indicates the percentage of bone formation and the statistical analyzes were performed with the paired t-test (p-value: *p<0.05; **p<0.01 and ***p<0.001).

### DETAILED DESCRIPTION

In one aspect, the invention relates to a pharmaceutical composition comprising:
a miPEP from 3 to 500 amino acids encoded by a nucleotide sequence contained in the primary transcript of a miR, or a fragment of said miPEP, said miPEP being capable of modulating the accumulation of said miR in a eukaryotic cell, and
a pharmaceutically acceptable excipient.

In the invention, the terms “microARN”, “non coding microARN” and “miR” are equivalent and may be used interchangeably. They define small molecules of RNA of about 21 nucleotides, which are not translated and do not lead to a peptide or a protein.

However, in this mature form, the microRNAs perform a function of regulation of certain genes via post-transcriptional mechanisms, for example by means of the RISC complex.

The primary transcript of the microRNA or “pri-miR” corresponds to the RNA molecule obtained directly from transcription of the DNA molecule. Generally, this primary transcript undergoes one or more post-transcriptional modifications, involving for example a particular structure of the RNA or cleavage of certain portions of the RNA, and which lead to the precursor form of the microRNA or “pre-miR”, then to the mature form of the microRNA or “miR”.

The terms “micropeptides” and “miPEPs” (microRNA encoded PEPtides) are equivalent and may be used interchangeably. They define a peptide that is encoded by an open reading frame present on the primary transcript of a microRNA, and which is capable of modulating the accumulation of said microRNA. The micropeptides within the meaning of the present invention are not to be understood as necessarily being small peptides, as “micro” does not correspond to the size of the peptide.

According to the invention, a miPEP can also be considered as a transcription modulator, and in particular a transcription activator. Such a transcription modulator can operate at the transcription level to modulate the accumulation of pri-miR, pre-miR and miR.

Taking into account the degeneracy of the genetic code, one and the same micropeptide may be encoded by several nucleotide sequences. Nucleotide sequences of this kind, differing from one another by at least one nucleotide but encoding one and the same peptide, are called “degenerate sequences”.

The terms “open reading frame” or “ORF” are equivalent and may be used interchangeably. They correspond to a nucleotide sequence in a DNA or RNA molecule that may potentially encode a peptide or a protein: said open reading frame begins with a start codon (the start codon generally encoding a methionine), followed by a series of codons (each codon encoding an amino acid), and ends with a stop codon (the stop codon not being translated).

In the invention, the ORFs may be called specifically “miORFs” when they are present on the primary transcripts of microRNA.

The miORFs may be contained in the 5′ or 3′ portion of said primary microAR transcript, preferably in the 5′ portion.

The 5′ or 3′ portions of the primary microRNA transcript correspond to the terminal portions of the RNA molecule that are cleaved during microRNA processing.

The miORFs as defined in the particular invention may have a size from 12 to 1503 nucleotides and may encode peptides from 3 to 500 amino acids.

In particular, the miORFs encode miPEPs having a size of:

3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 amino acids.

The miPEPs may especially have a size from 100 to 500 amino acids, from 101 to 500 amino acids, from 200 to 500 amino acids, from 300 to 500 amino acids, or from 400 to 500 amino acids.

According to the invention, a “miPEP fragment” corresponds to an N-terminal part, a C-terminal part or an internal part of the sequence of a miPEP.

In others words, a “miPEP fragment” corresponds to a part of a miPEP containing the first N-terminal amino acid, to a part of the miPEP containing the last C-terminal amino acid or to a part containing neither the first N-terminal amino acid or the last C-terminal amino acid.

In particular, a miPEP fragment has a size from 2 to 20 amino acids, preferably from 5 to 10 amino acids.

In particular, a miPEP fragment has a size of 2, 3, 4, 5, 6, 7, 8, 9, 10, 11, 12, 13, 14, 15, 16, 17, 18, 19 or 20 amino acids.

Preferably, a miPEP fragment has a size of 10 amino acids and corresponds to the N-terminal portion of a miPEP.

In the present invention, a miPEP fragment may be referred to as “miPEP//”.

In the invention, “accumulation” means the production of a molecule, such as a microRNA or a micropeptide, in the cell.

The accumulation of a microRNA or a micropeptide can be determined using assay methods known to those skilled in the art, such as for example by RT-qPCR or by the quantification of the fluorescence due to the fusion of a peptide or protein to a fluorescent marker.

Thus, “modulation” of the accumulation of a molecule in a cell corresponds to a modification of the quantity of this molecule present in the cell.

Moreover, the effect of a miPEP can be observed through the modulation of the accumulation of the miR, but also through the modulation of the accumulation of the corresponding pri-miR or pre-miR.

In one embodiment, the invention relates to a composition as defined above, wherein the modulation of the accumulation of said microRNA is a decrease or an increase in the accumulation of said microRNA, in particular an increase.

A “decrease in the accumulation” corresponds to a decrease in the quantity of said molecule in a cell relative to a control cell. Conversely, an “increase in the accumulation” corresponds to an increase in the quantity of said molecule in a cell relative to a control cell.

According to the invention, a miPEP is capable of modulating the accumulation of pri-miR, pre-miR and miR. Therefore, if a primary transcript contains more than one miR, a same miPEP encoded by said primary transcript is capable of modulating the accumulation of at least one of the miRs present on the primary transcript.

In a particular embodiment, the invention relates to a pharmaceutical composition comprising:
a miPEP from 3 to 500 amino acids encoded by a nucleotide sequence contained in the primary transcript of a miR, or a fragment of said miPEP, said miPEP being capable of modulating the accumulation of said miR in a eukaryotic cell, which miR regulates expression of at least one gene involved in a pathology,
a pharmaceutically acceptable excipient.

According to the invention, said pharmaceutical composition comprises at least one miPEP but may also comprise a mixture of several miPEPs.

In a nonlimiting manner, said pharmaceutical composition may for example comprise 2, 3, 4, 5, 6, 7, 8, 9 or 10 miPEPs.

In a particular embodiment, the invention relates to a pharmaceutical composition as defined above, wherein said miPEP is selected from the group of miPEPs consisting of SEQ ID NO: 2q-1, q varying from 1 to 5 875.

In a particular embodiment, the invention relates to a pharmaceutical composition as defined above, wherein said miPEP is selected from the group of miPEPs consisting of:

miPEP 145-2
(MVGLNPPLWQETGEYT, SEQ ID NO: 11,745),
miPEP 125a-1
(MSLCLSPSLTPTPGSTGPPHTMLPVSRSLRPFNL, SEQ ID
NO: 11,747), ant
miPEP 15a-16-1
(MFKHRFFYMHSFFPERKYFLYSLGANVCLKKIKPWSKVAAHN
GLWILKRCRPYCAASKIQGSDLLKKIYFFLFIALMIAMSAVP,
SEQ ID NO: 11,749).

In a particular embodiment, the invention relates to a pharmaceutical composition as defined above, wherein said miPEP fragment is a fragment of miPEP 145-2, preferably consisting of

SEQ ID NO: 11,751
(MVGLNPPLWQ).

In a particular embodiment, the invention relates to a pharmaceutical composition as defined above, wherein said miPEP fragment is the miPEP//145-2 consisting of SEQ ID NO: 11 751

(MVGLNPPLWQ).

In a particular embodiment, the invention relates to a pharmaceutical composition as defined above, wherein said miPEP fragment is a fragment of miPEP 125a-1, preferably consisting of

SEQ ID NO: 11,752
(MSLCLSPSLT).

In a particular embodiment, the invention relates to a pharmaceutical composition as defined above, wherein said miPEP fragment is the miPEP//125a-1 consisting of SEQ ID NO: 11 752

(MSLCLSPSLT).

In a particular embodiment, the invention relates to a pharmaceutical composition as defined above, wherein said miPEP fragment is a fragment of miPEP 15a-16-1, preferably consisting of SEQ ID NO: 11 753 (MFKHRFFYMH).

In a particular embodiment, the invention relates to a pharmaceutical composition as defined above, wherein said miPEP fragment is the miPEP//15a-16-1 consisting of SEQ ID NO: 11 753

(MFKHRFFYMH).

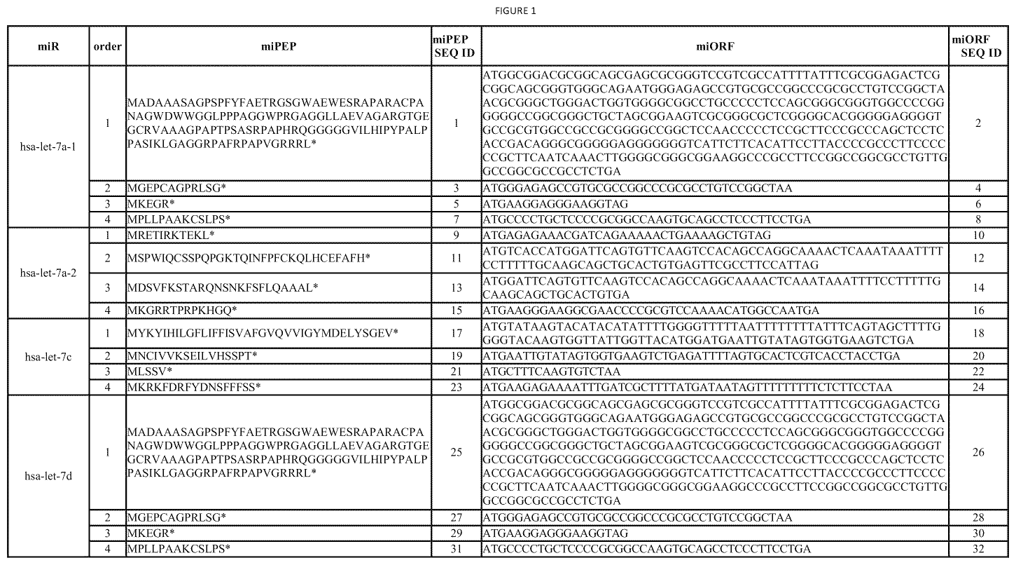

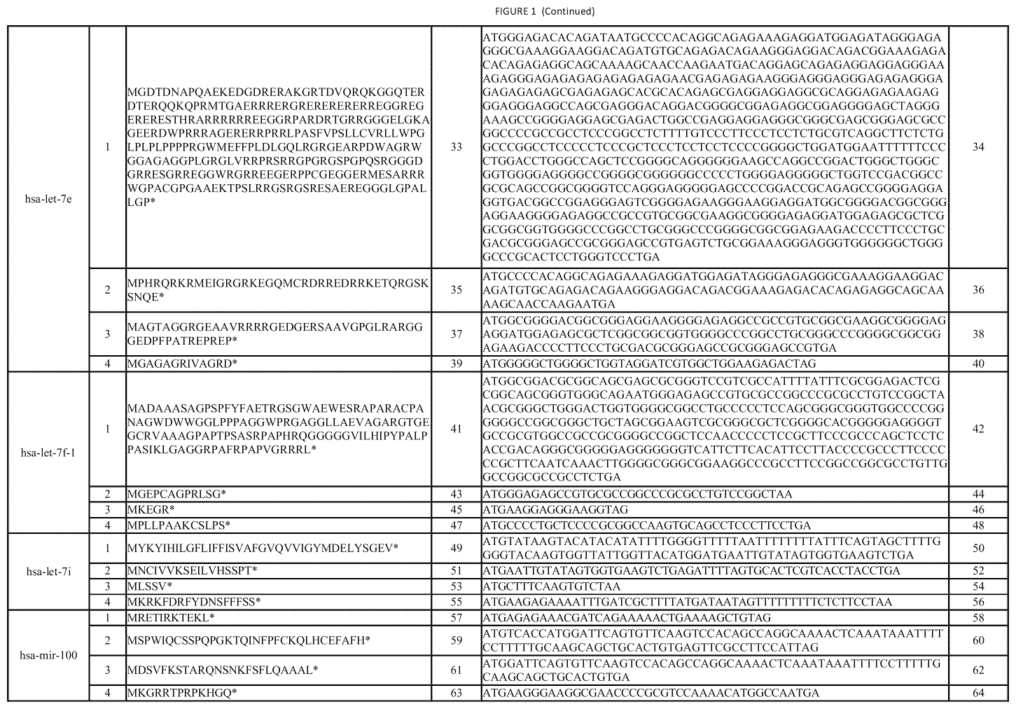

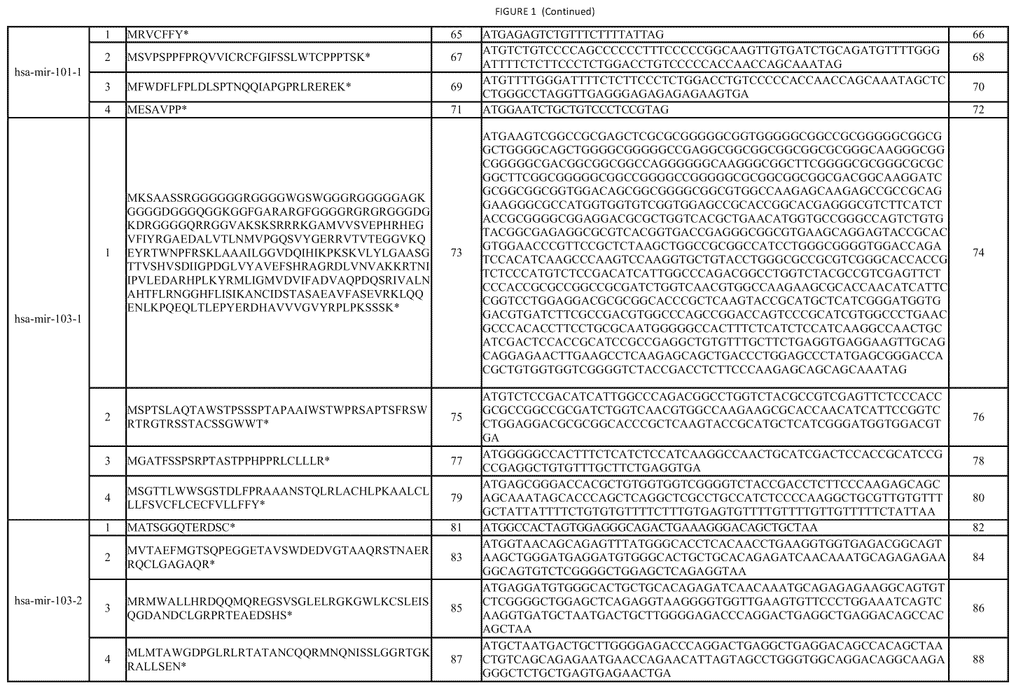

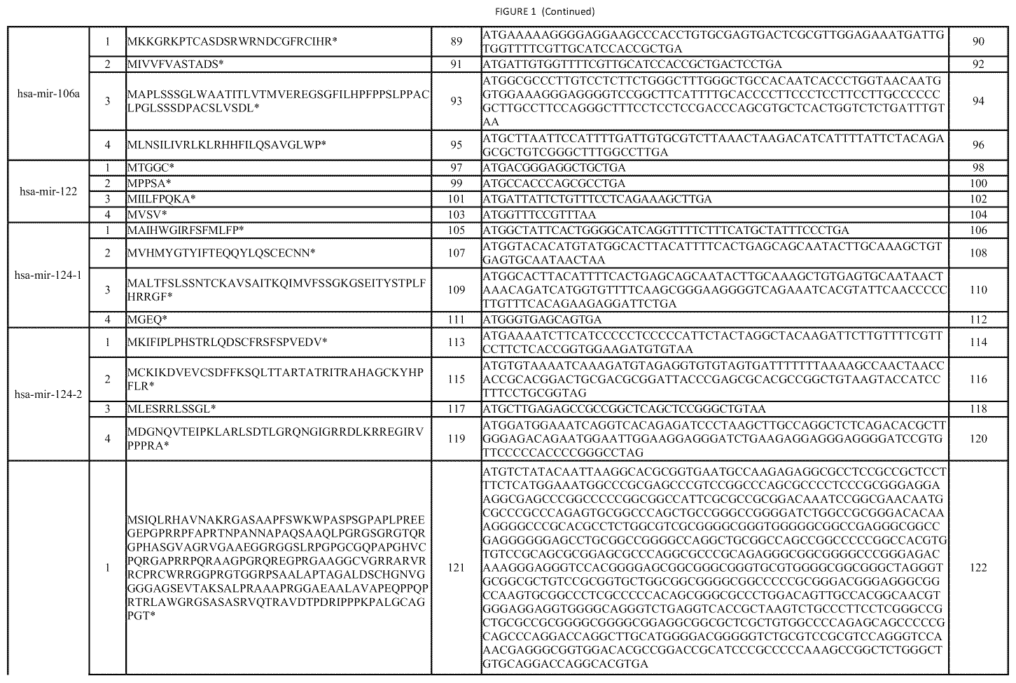

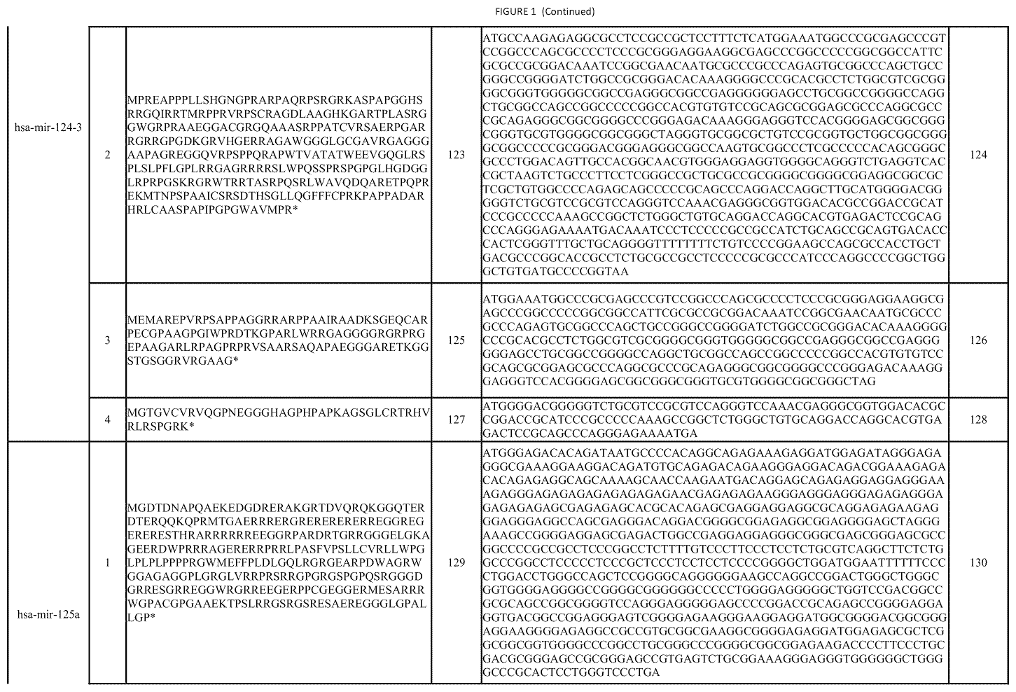

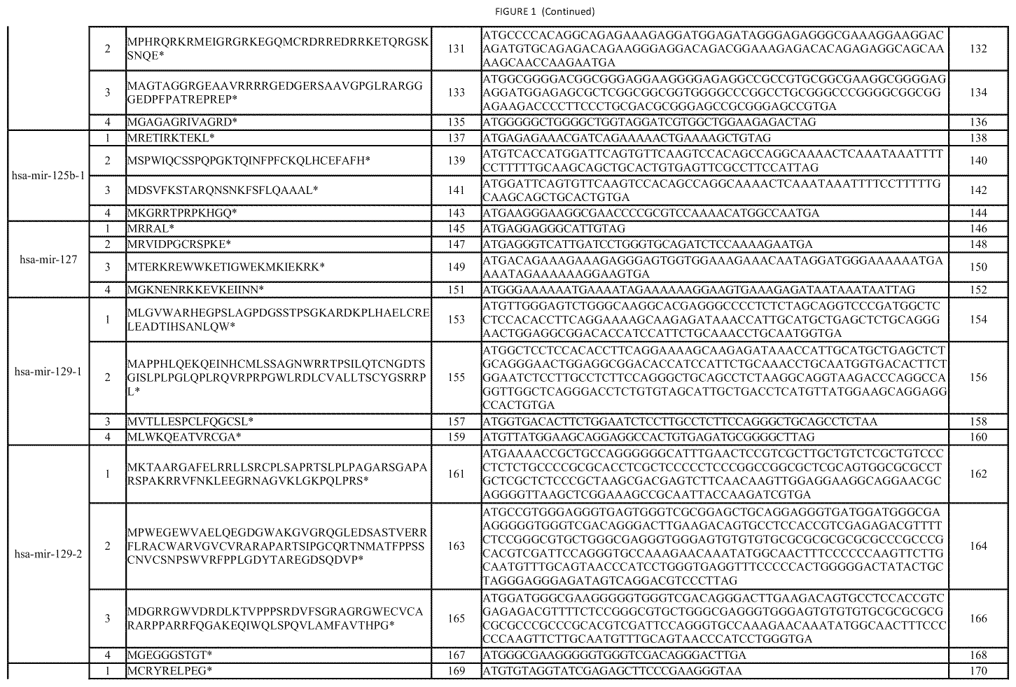

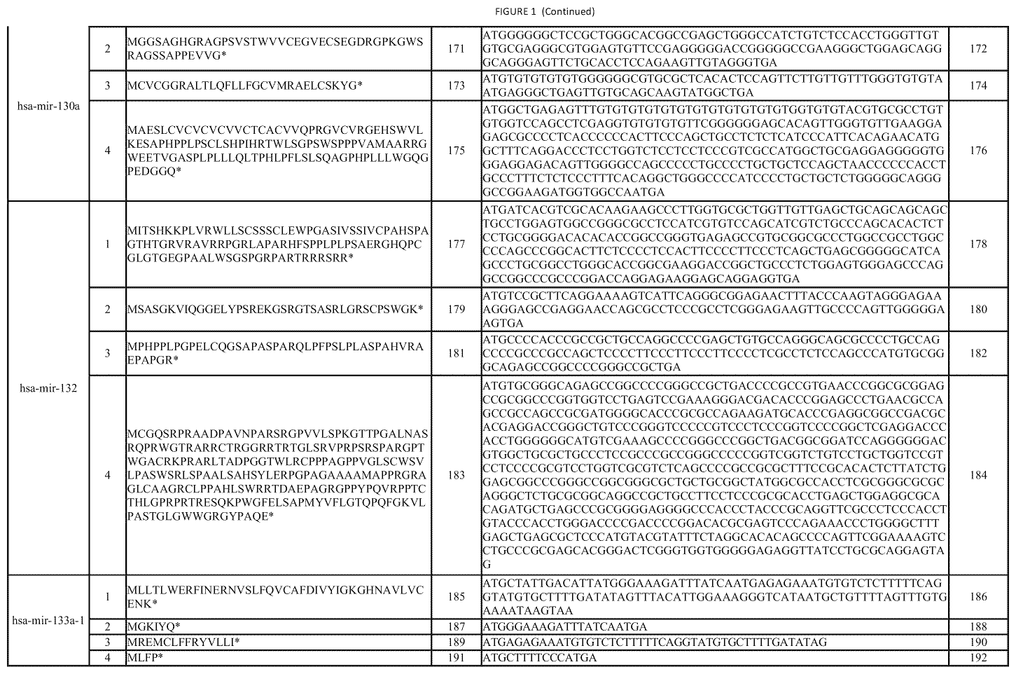

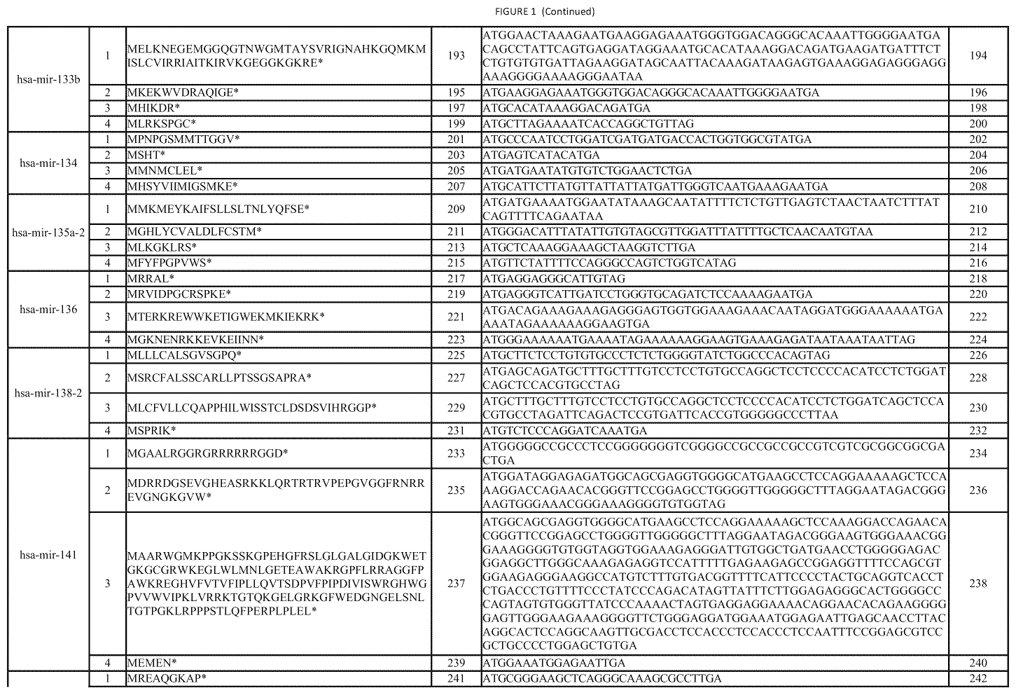

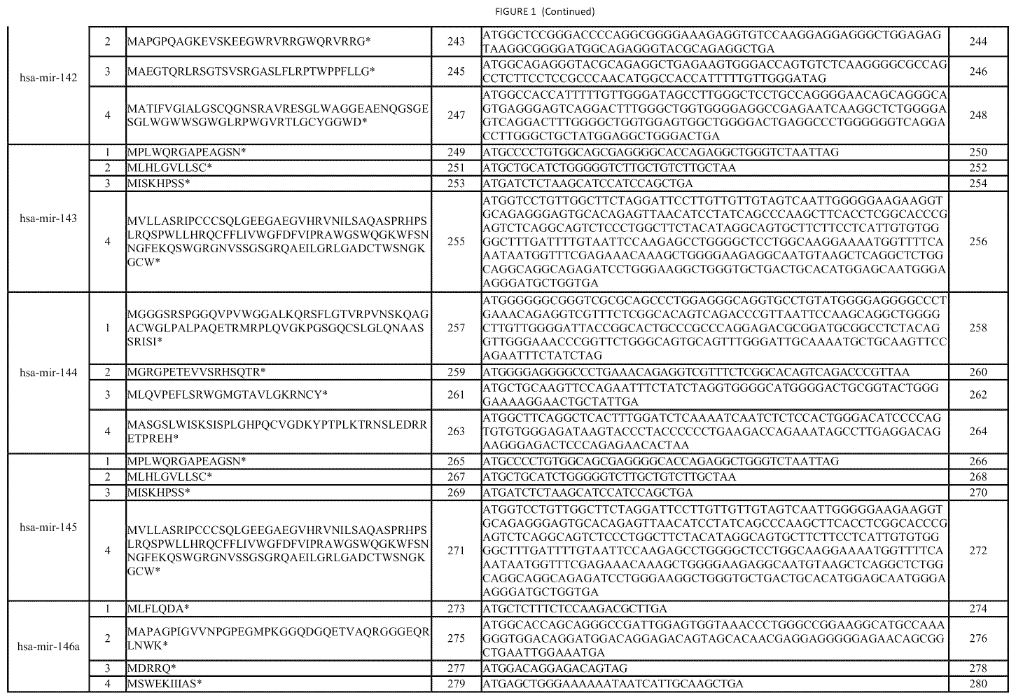

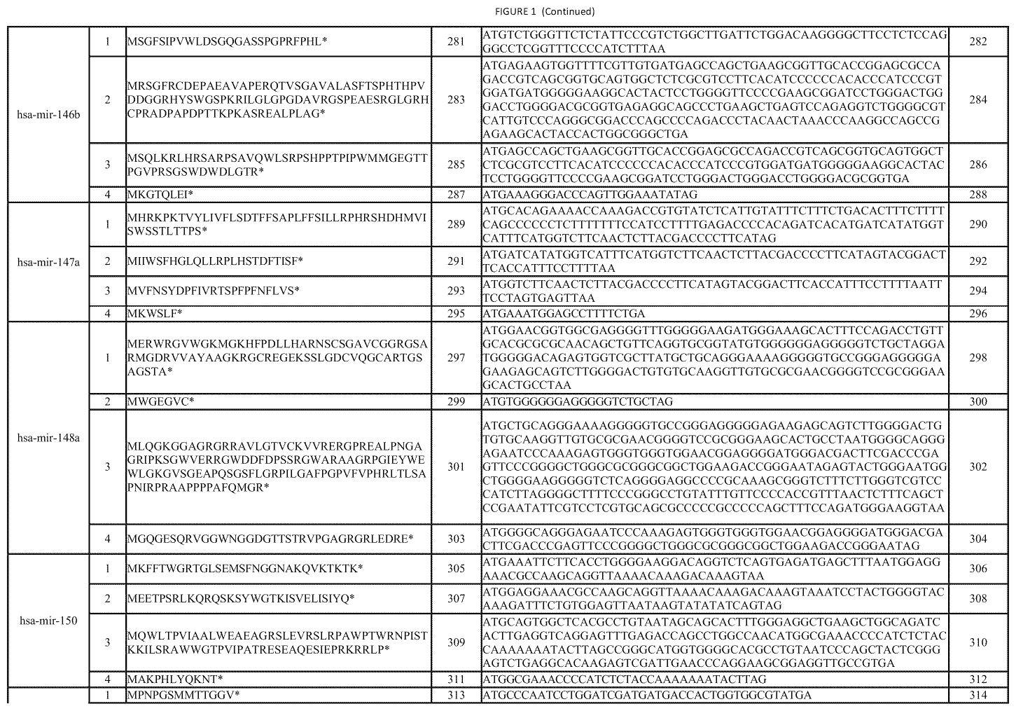

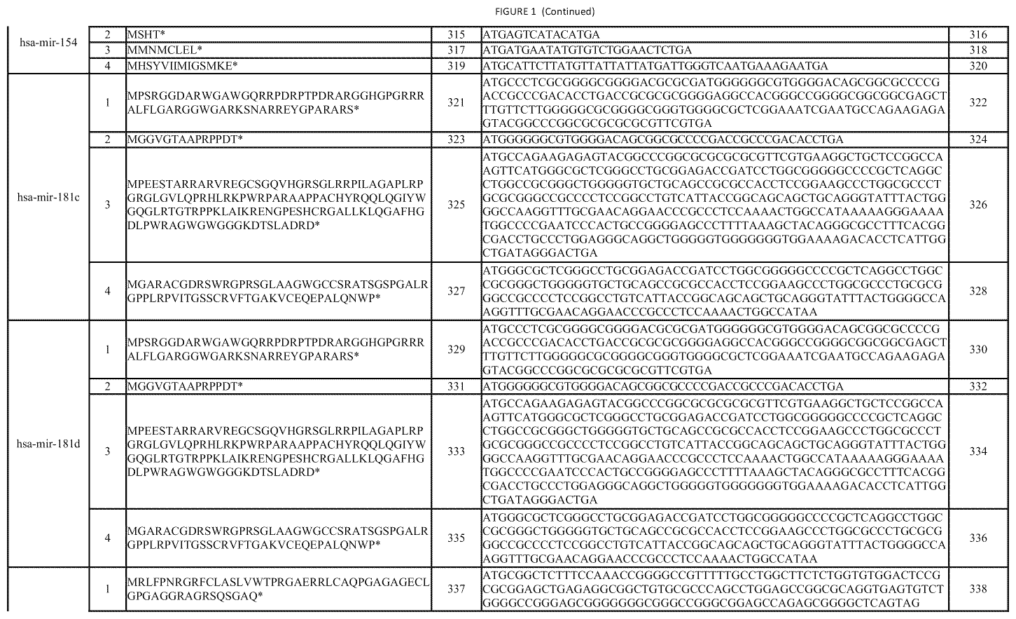

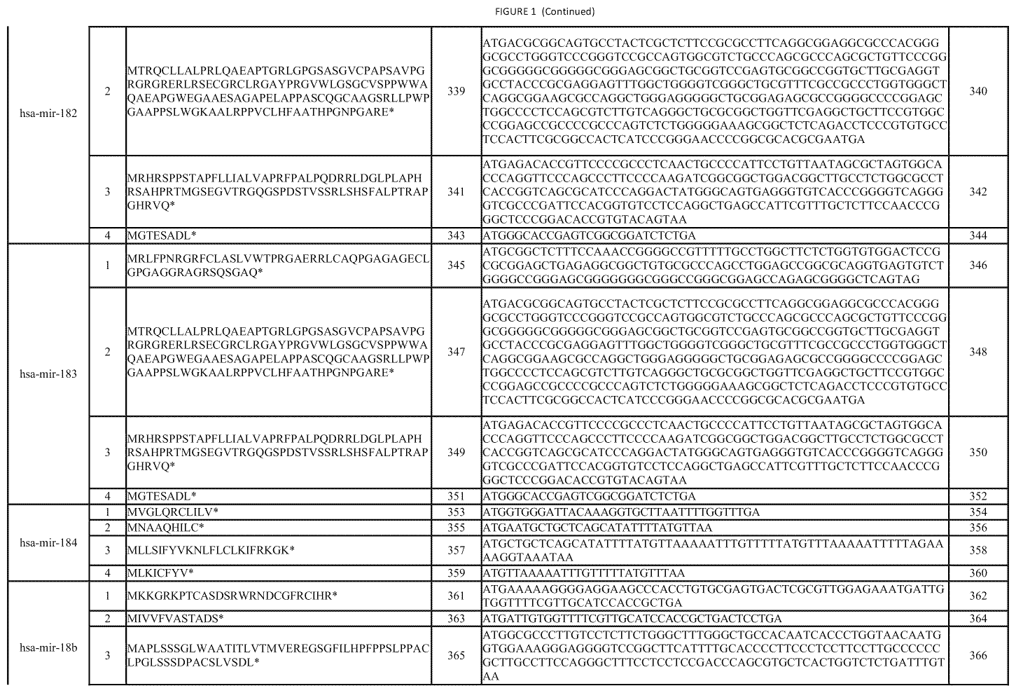

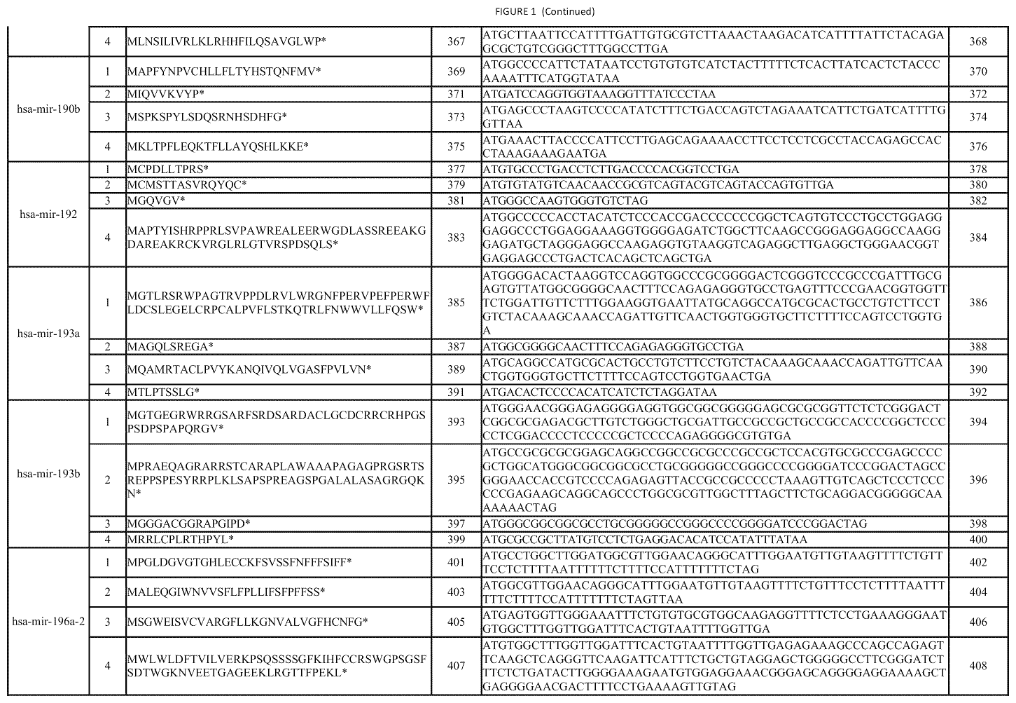

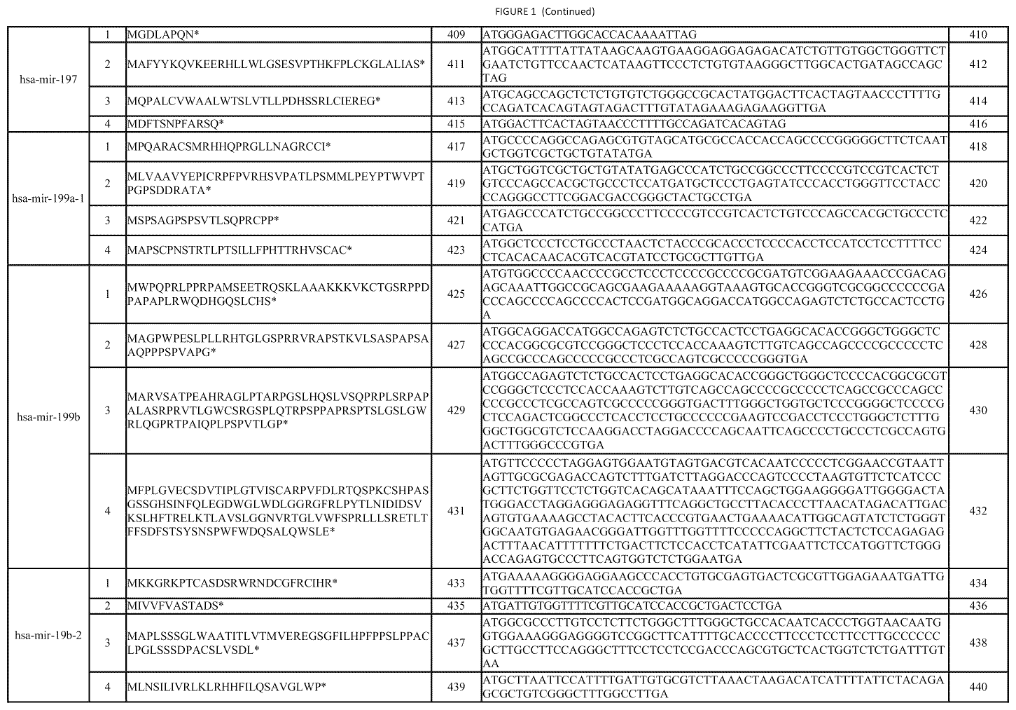

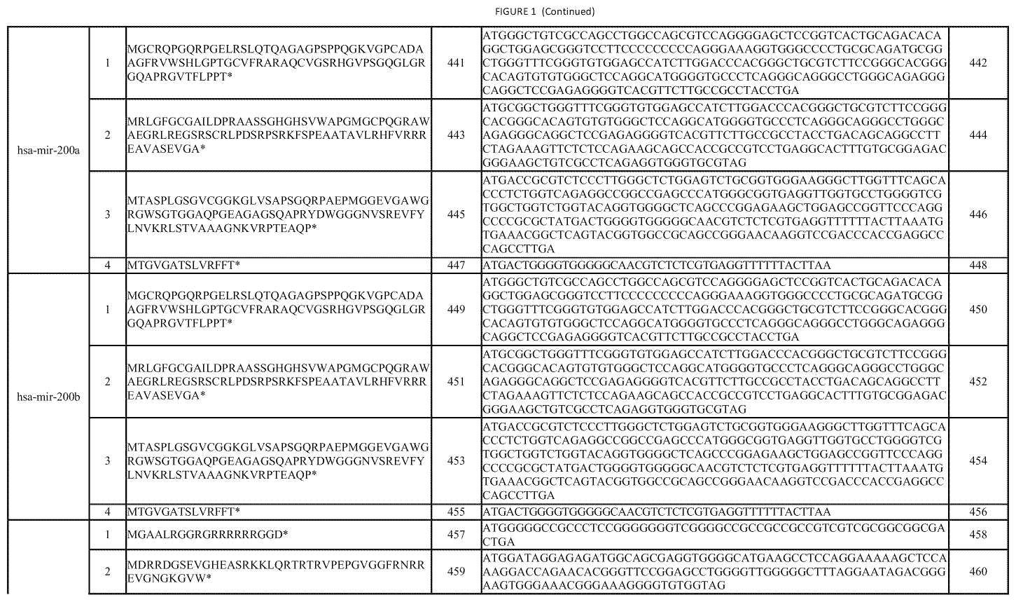

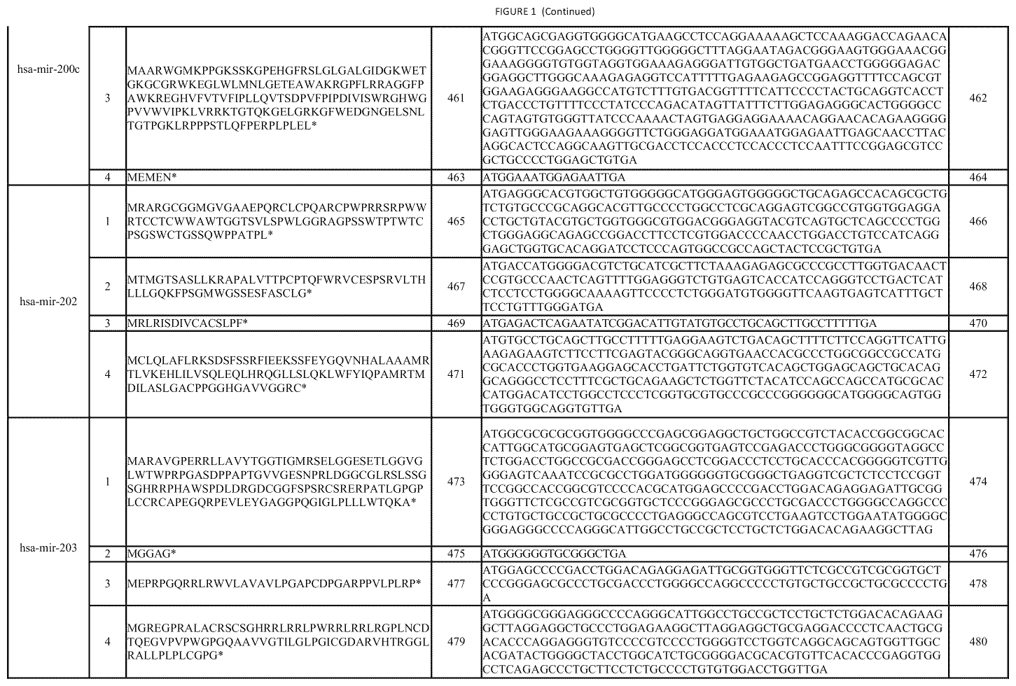

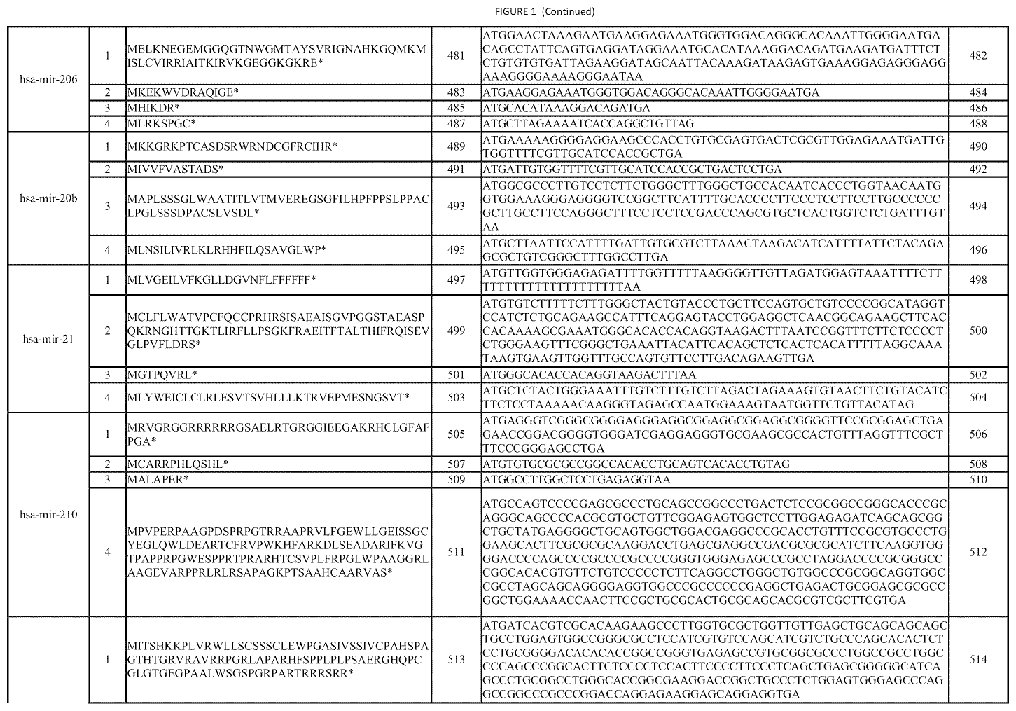

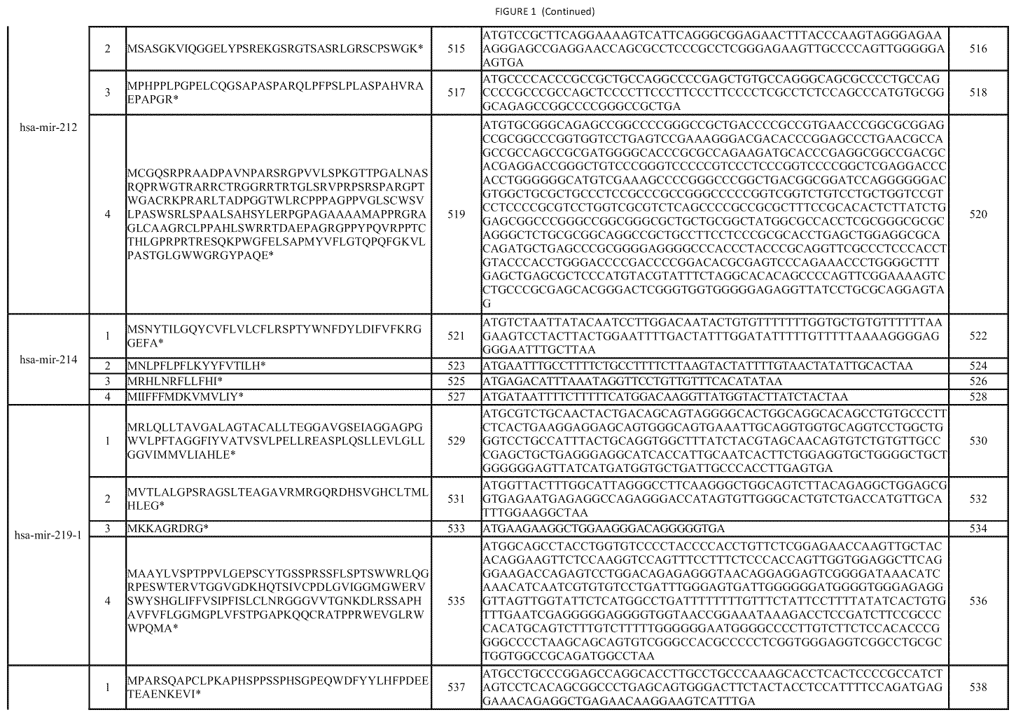

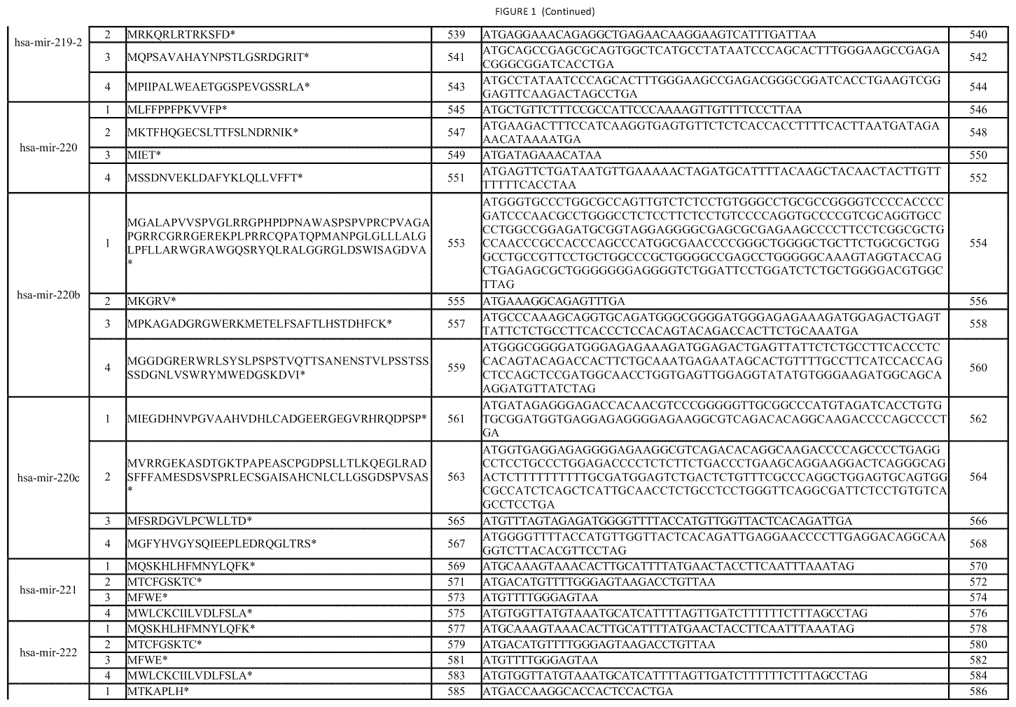

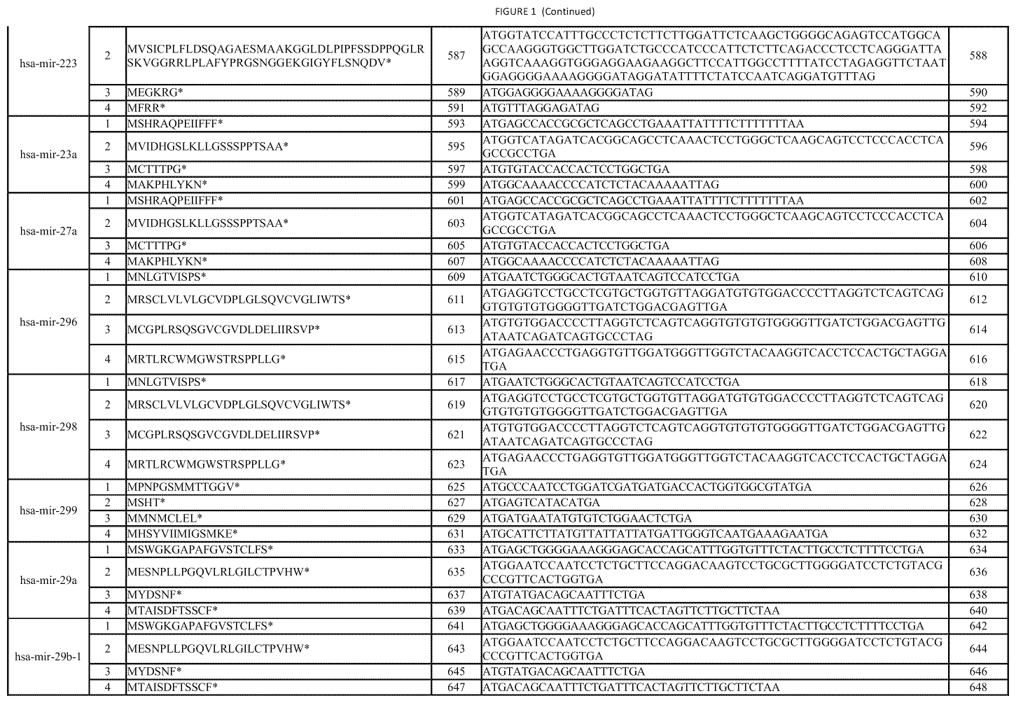

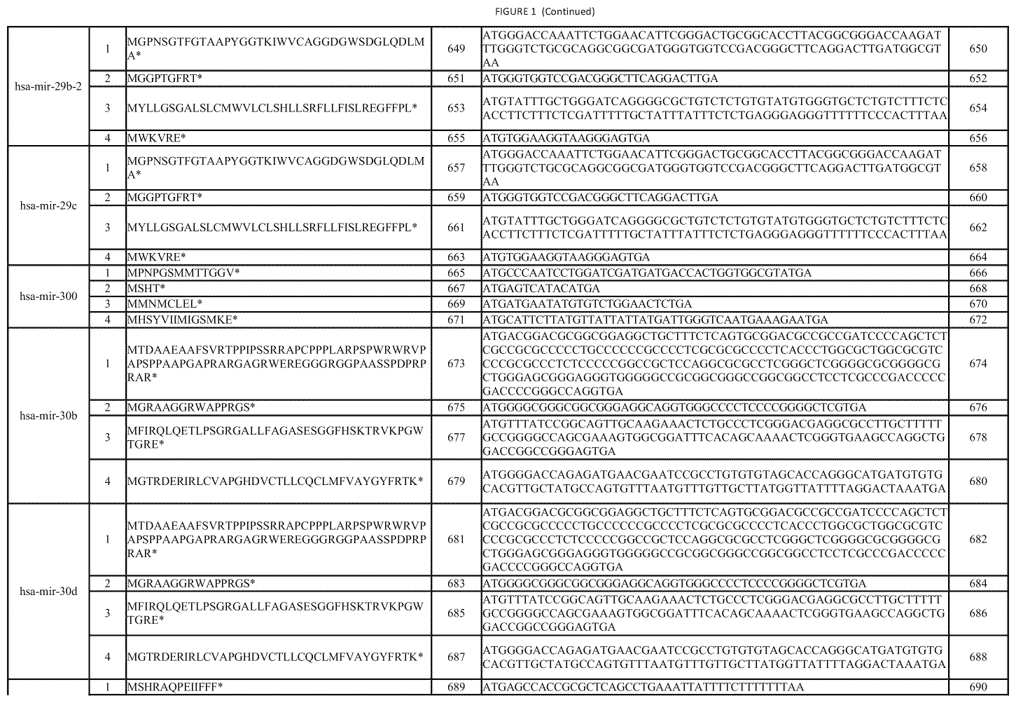

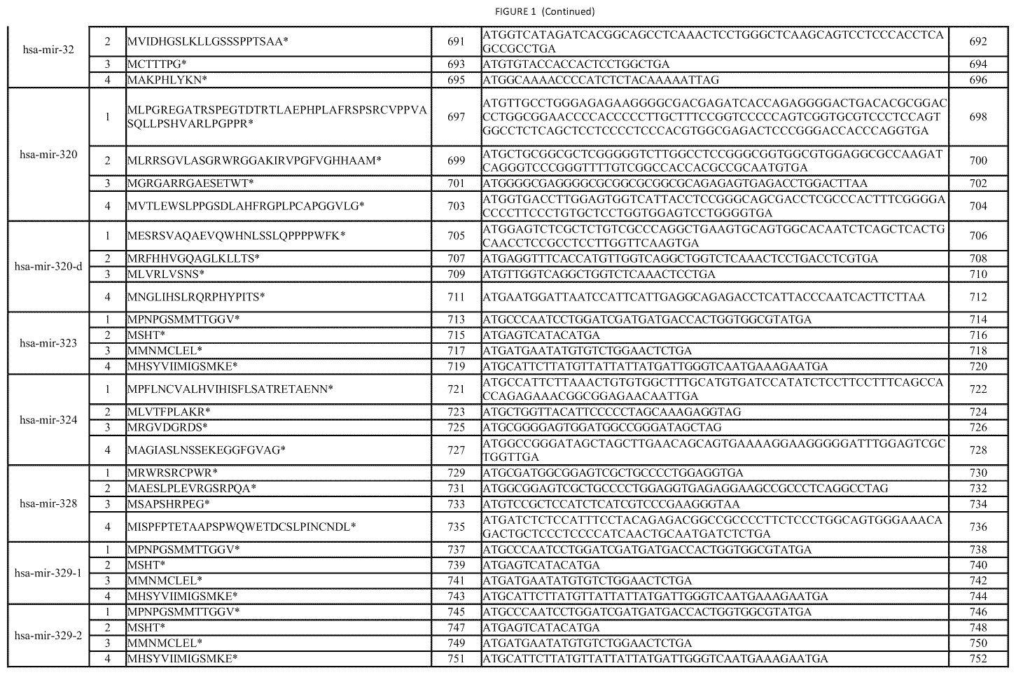

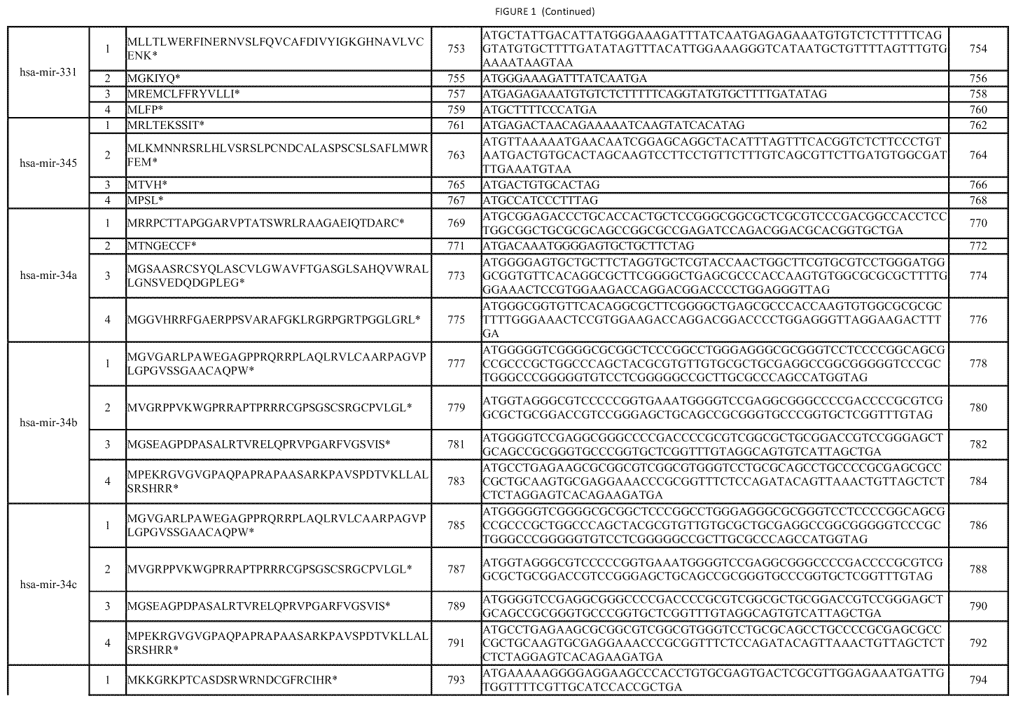

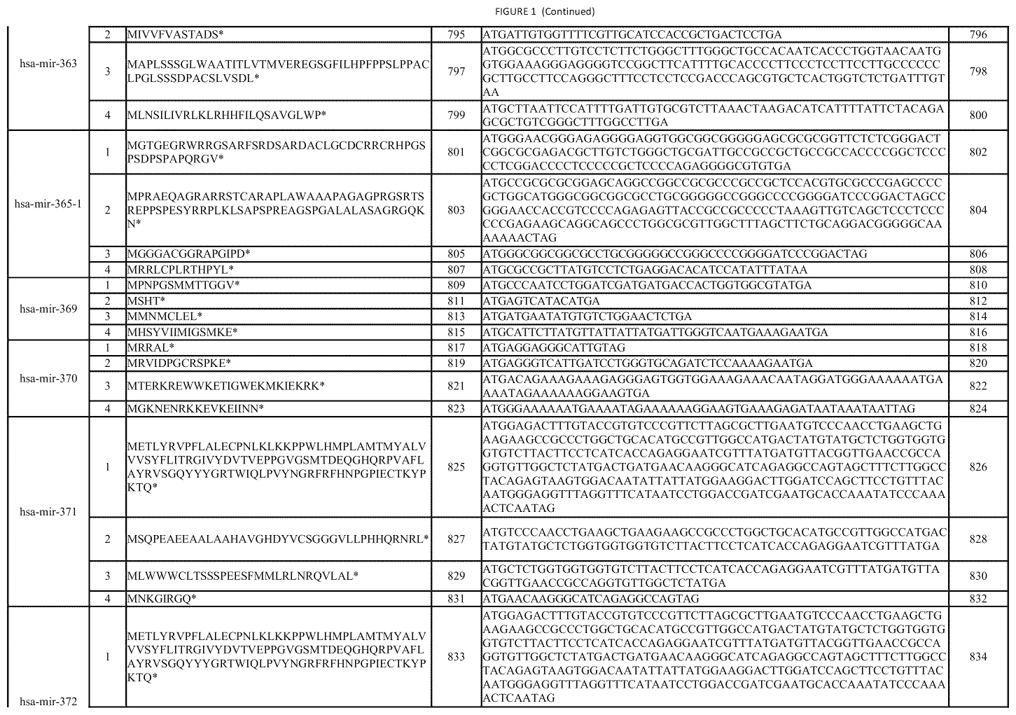

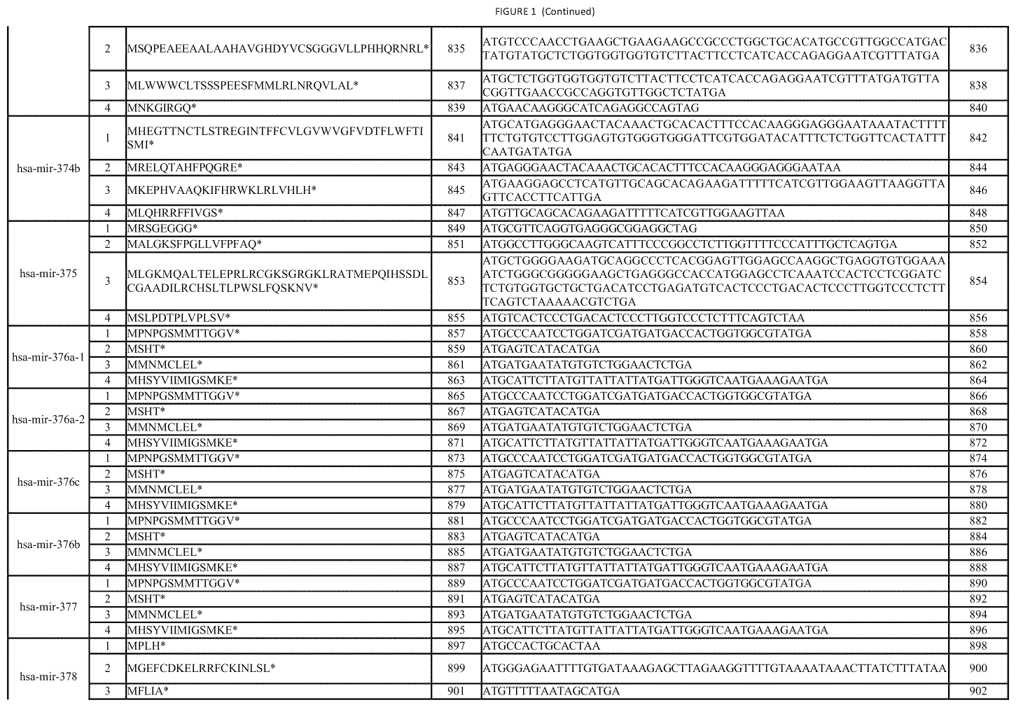

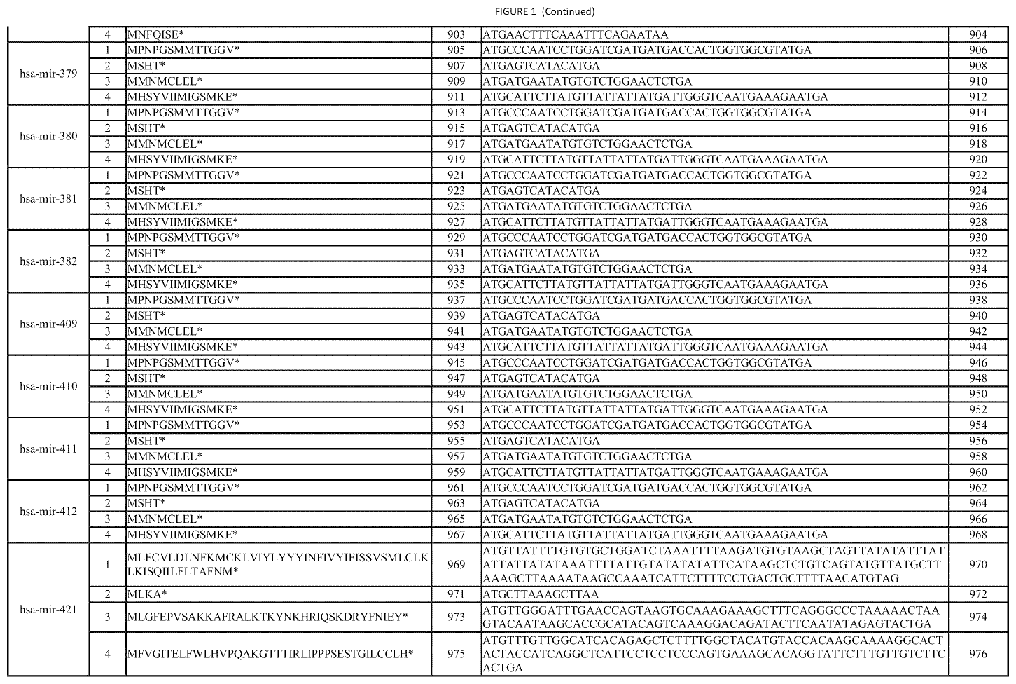

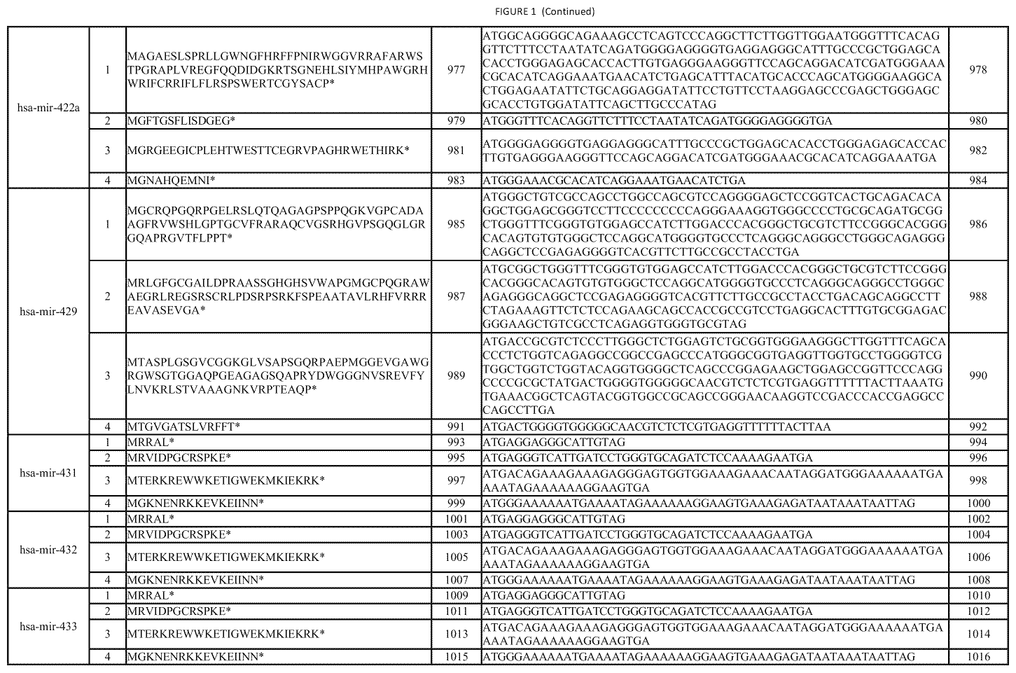

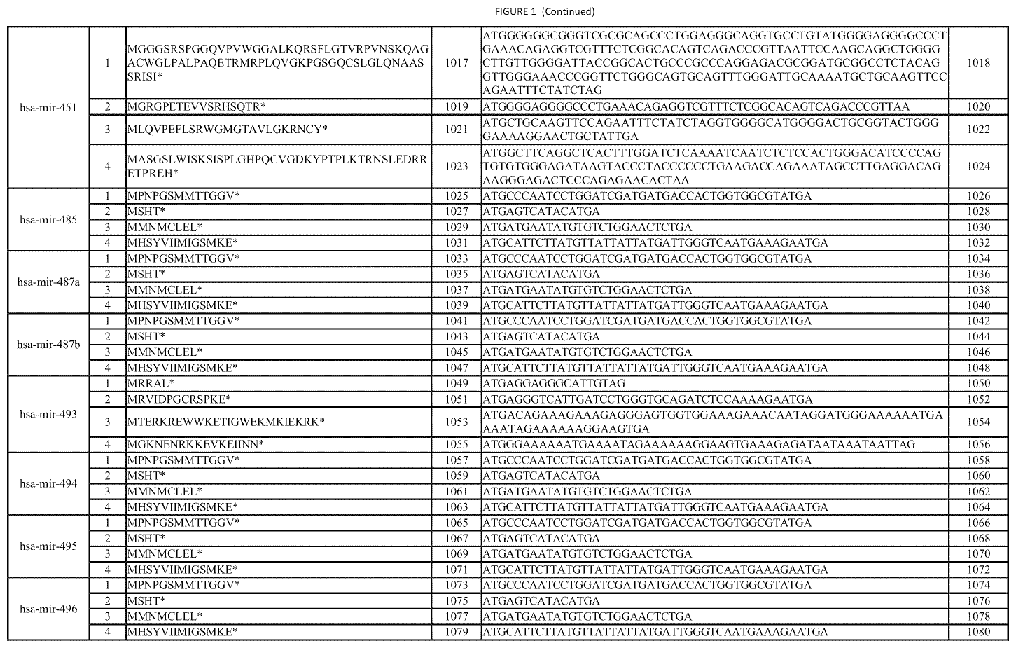

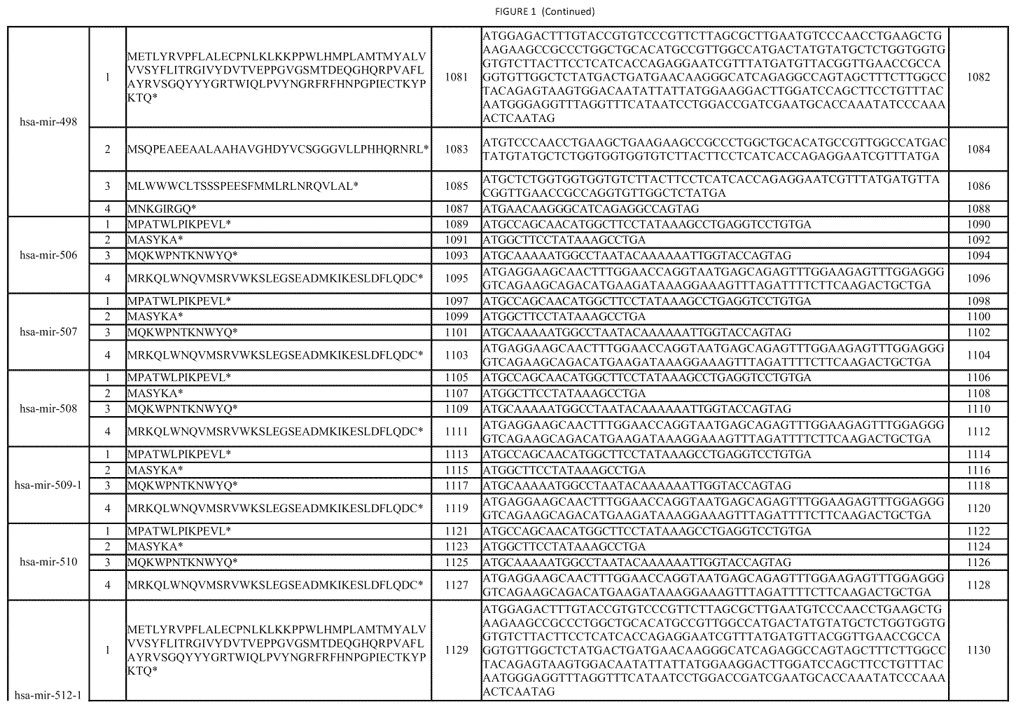

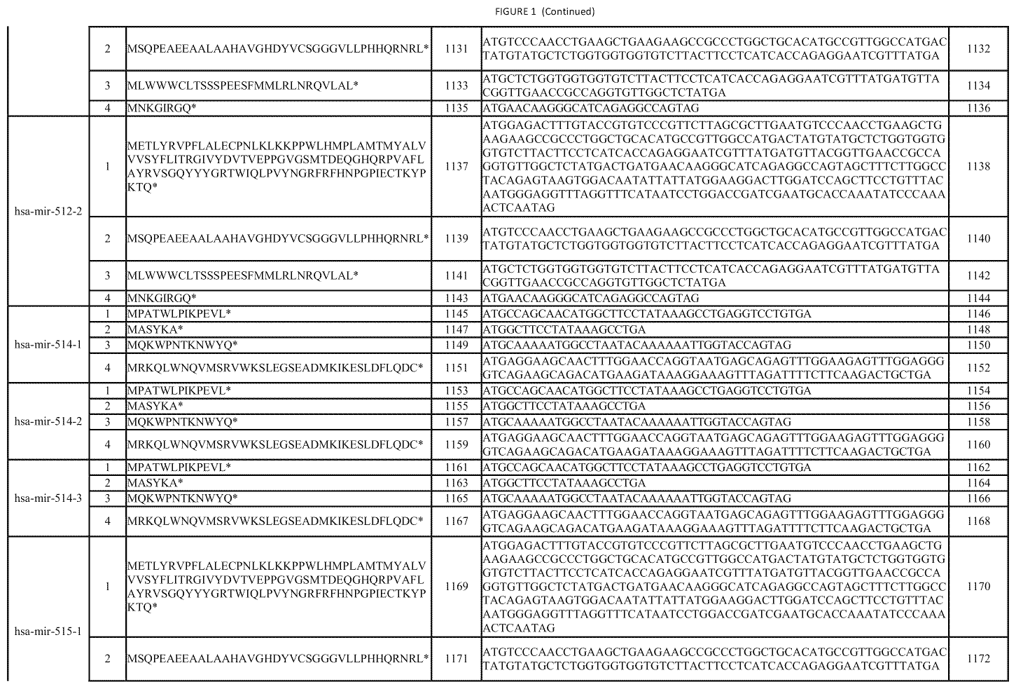

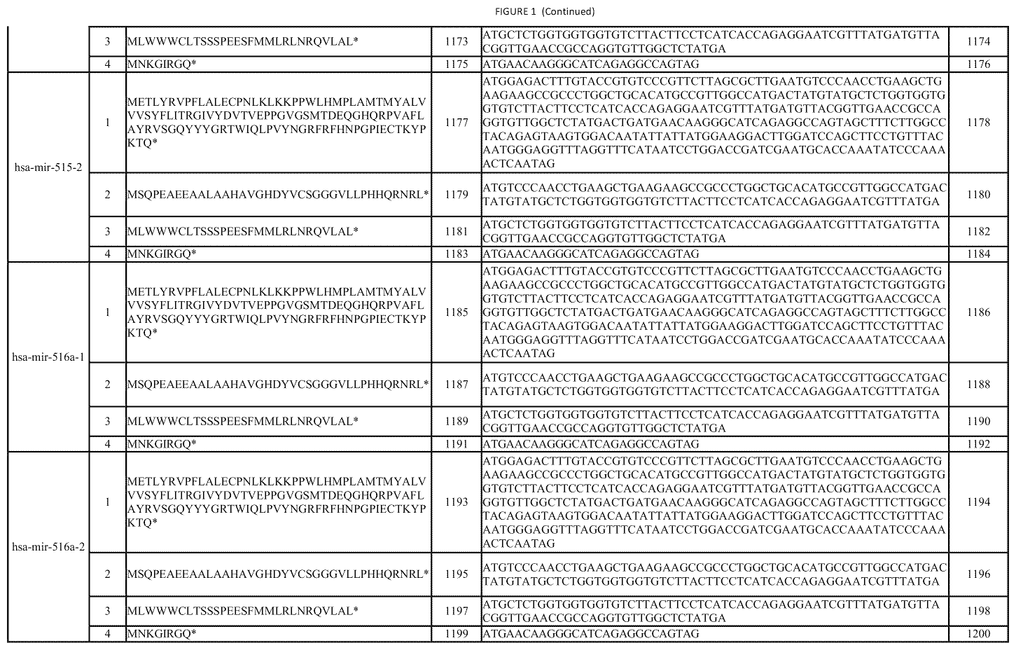

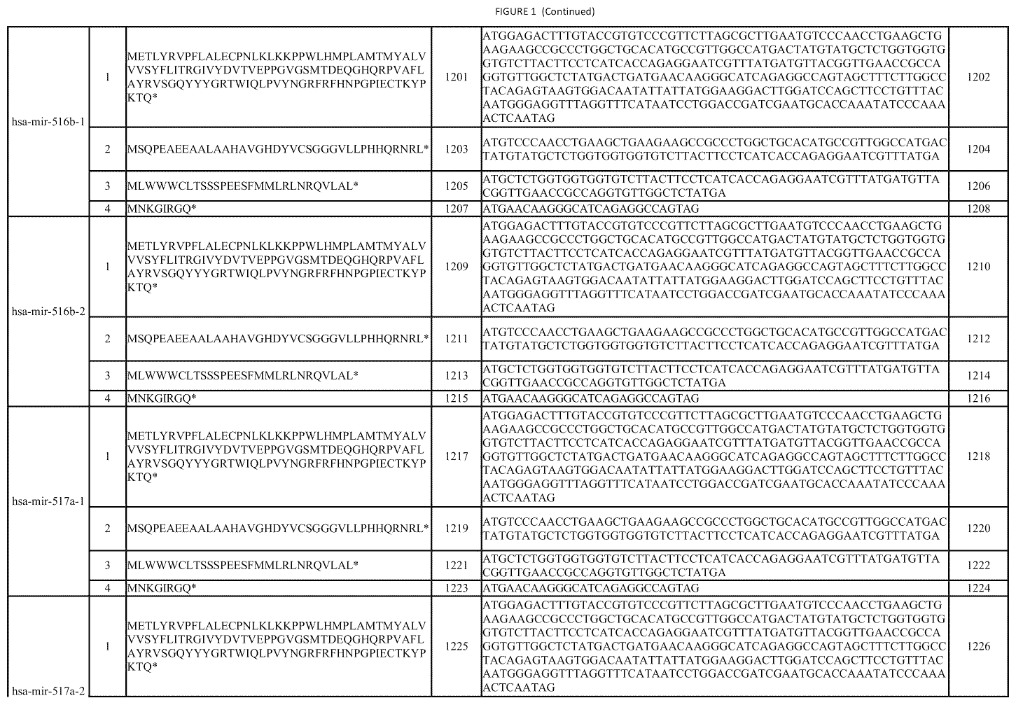

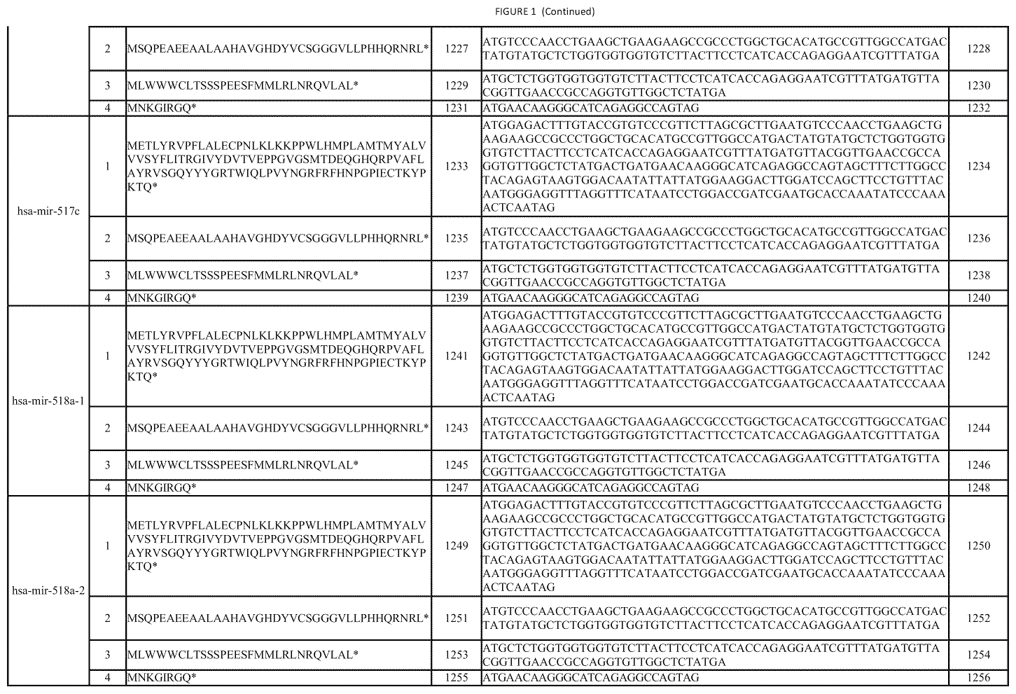

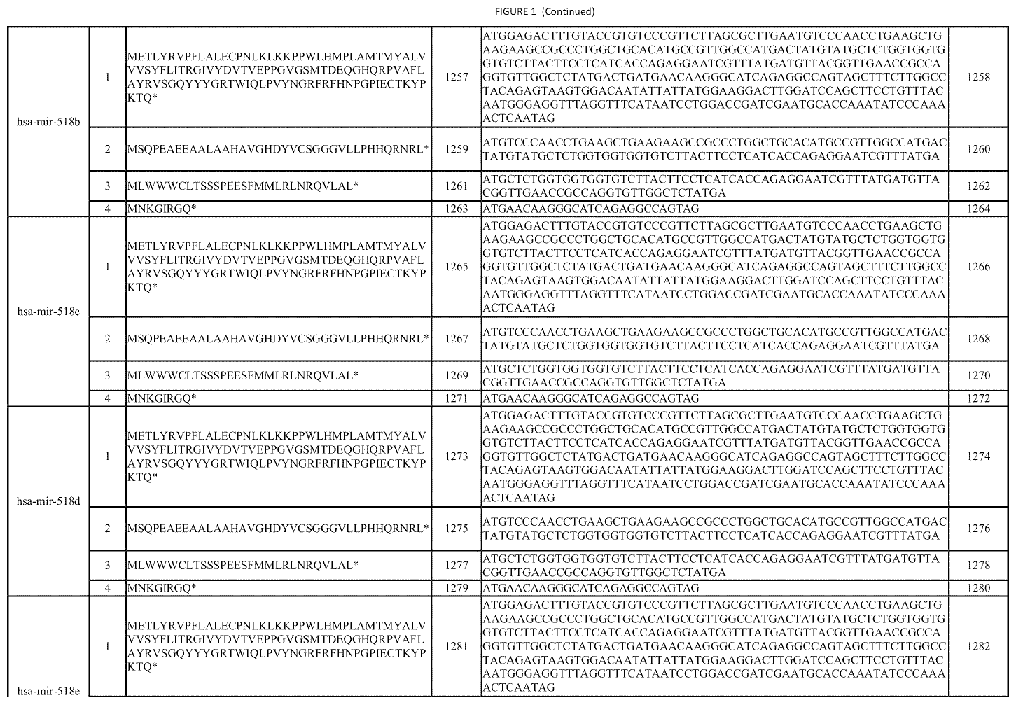

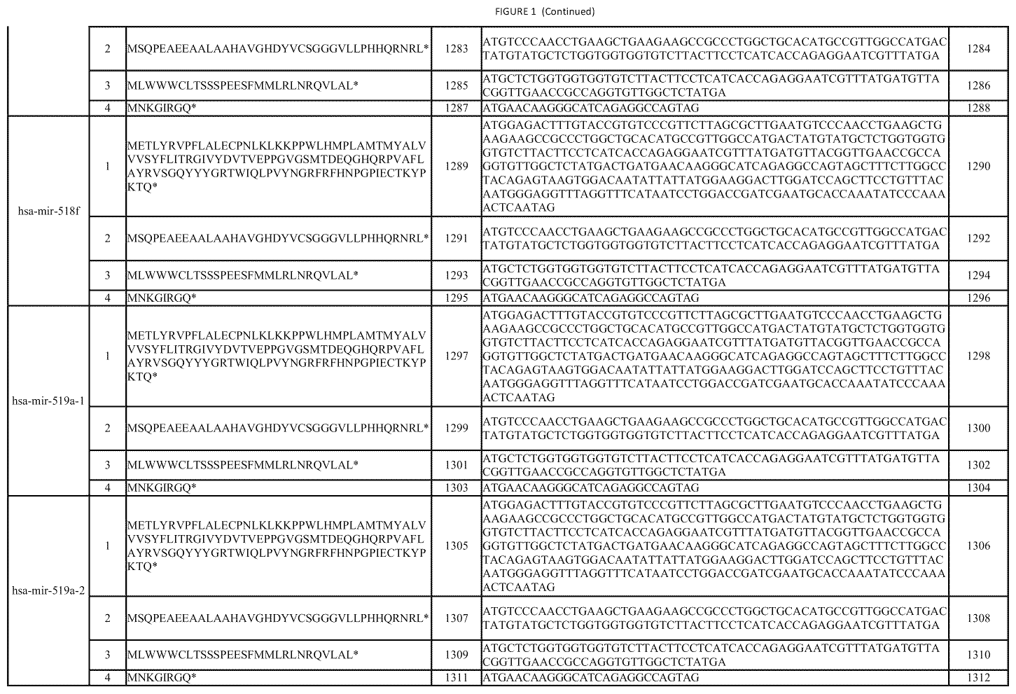

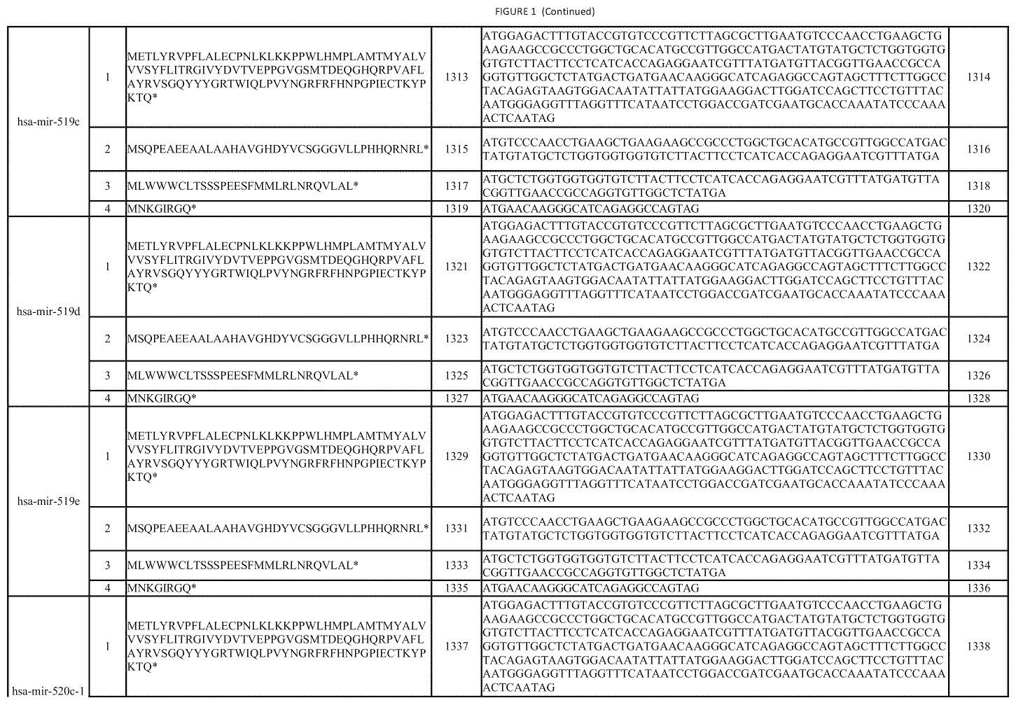

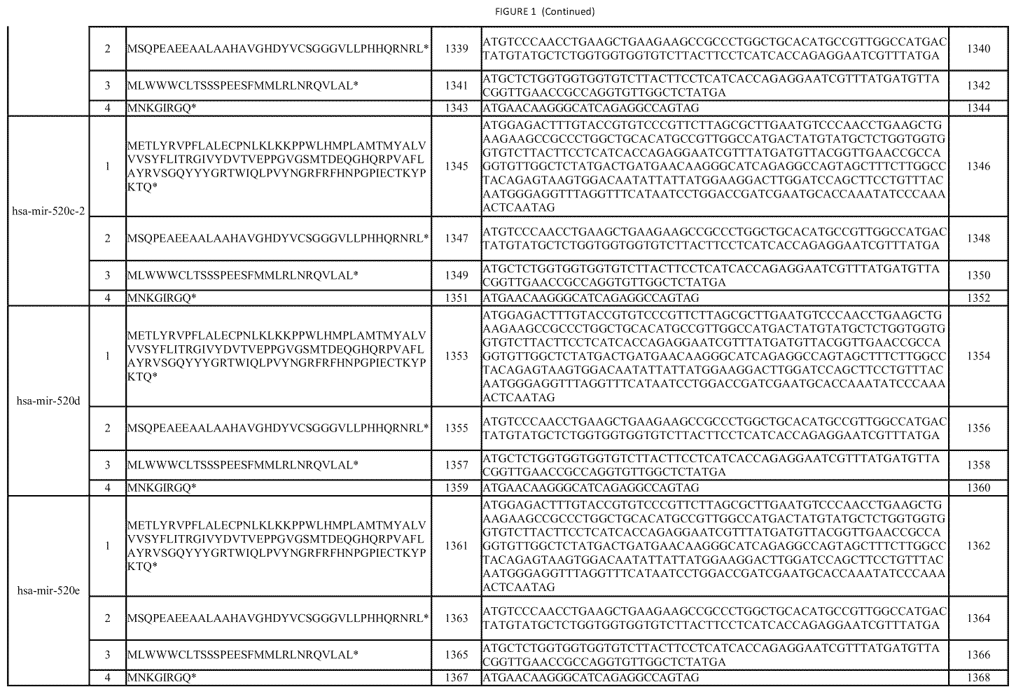

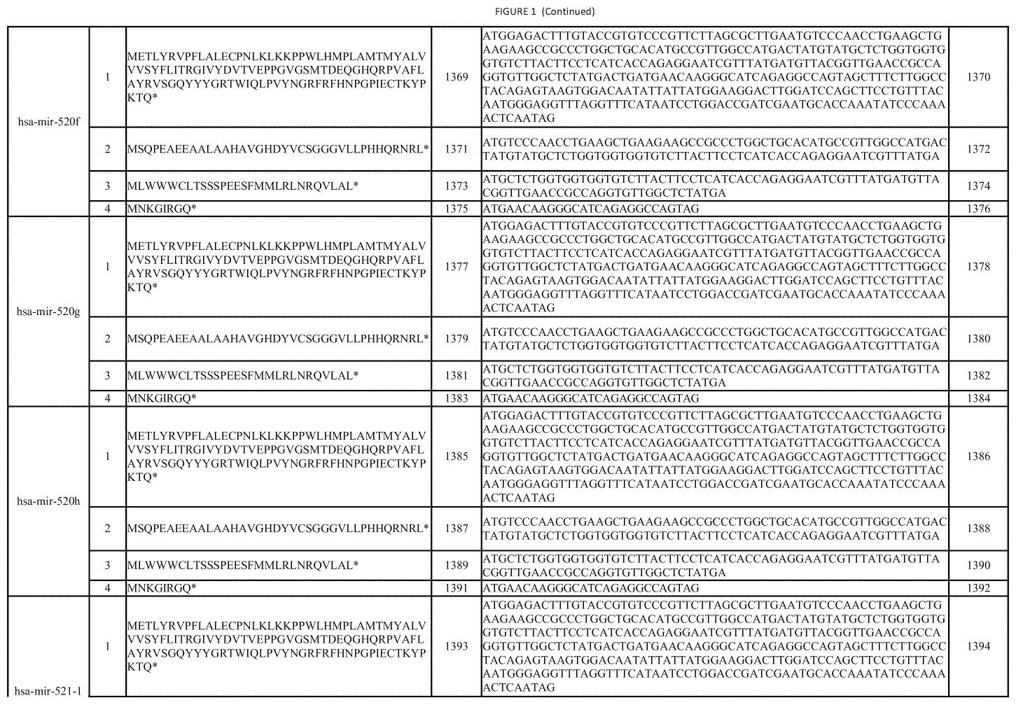

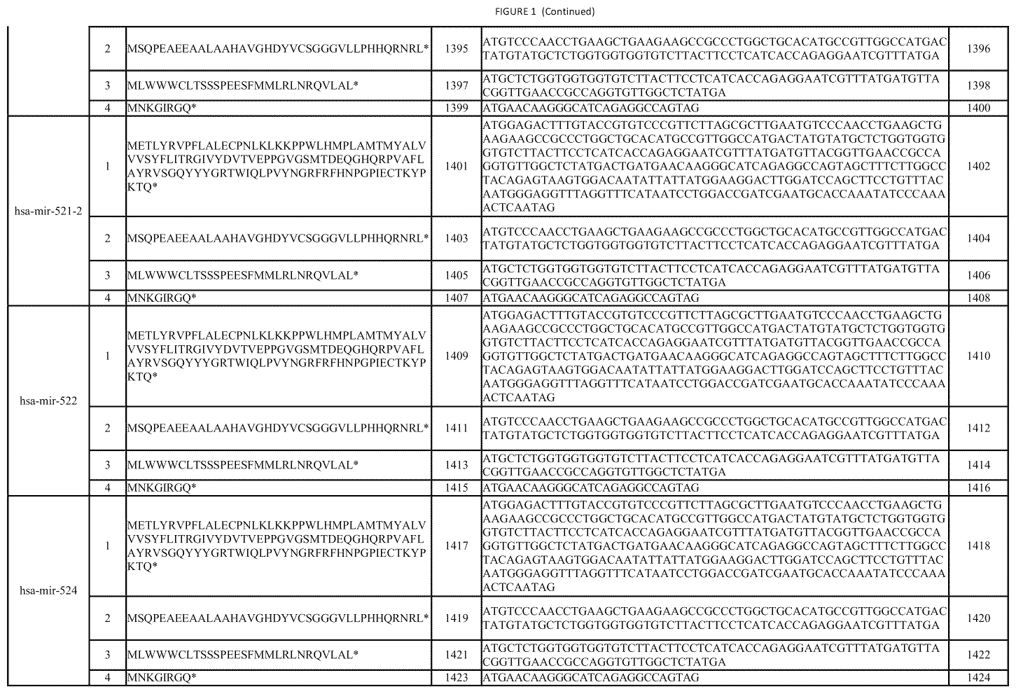

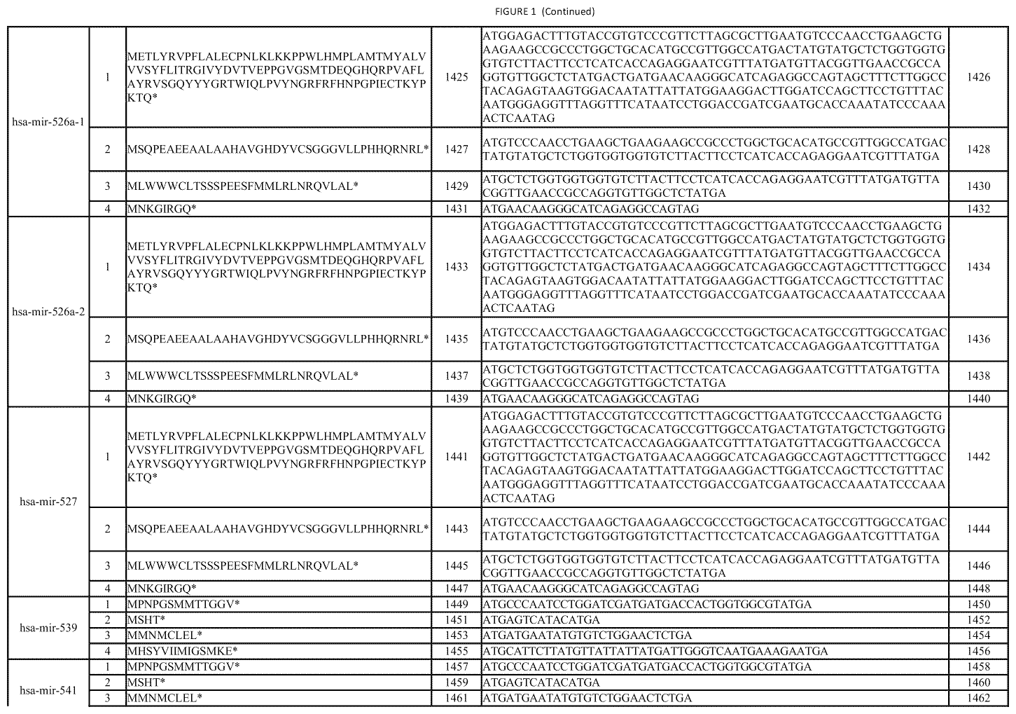

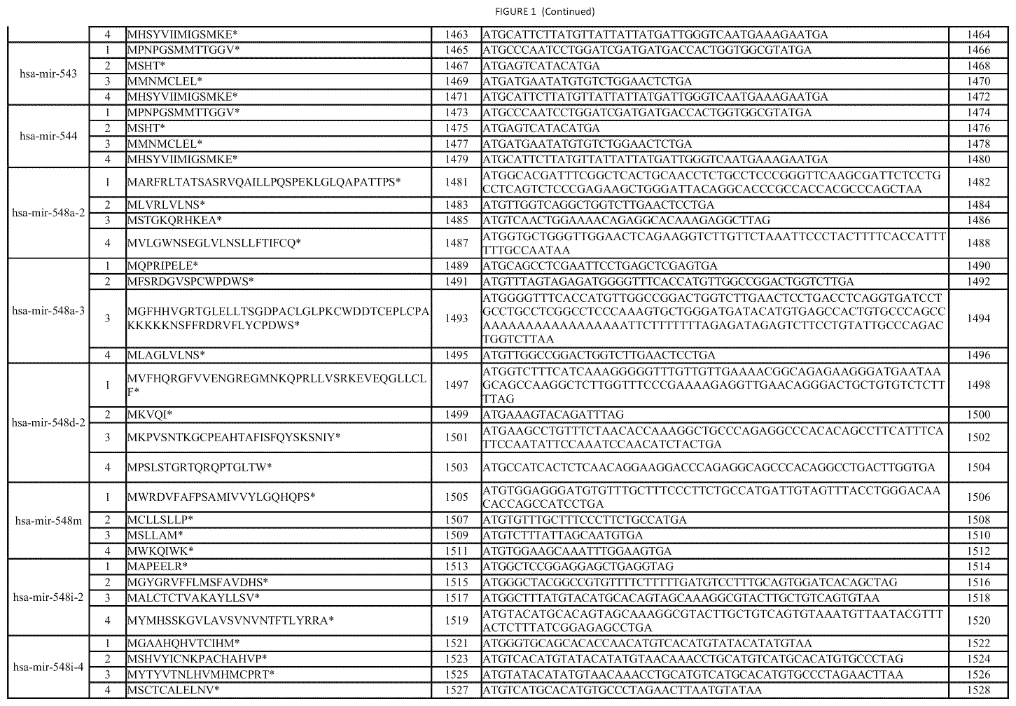

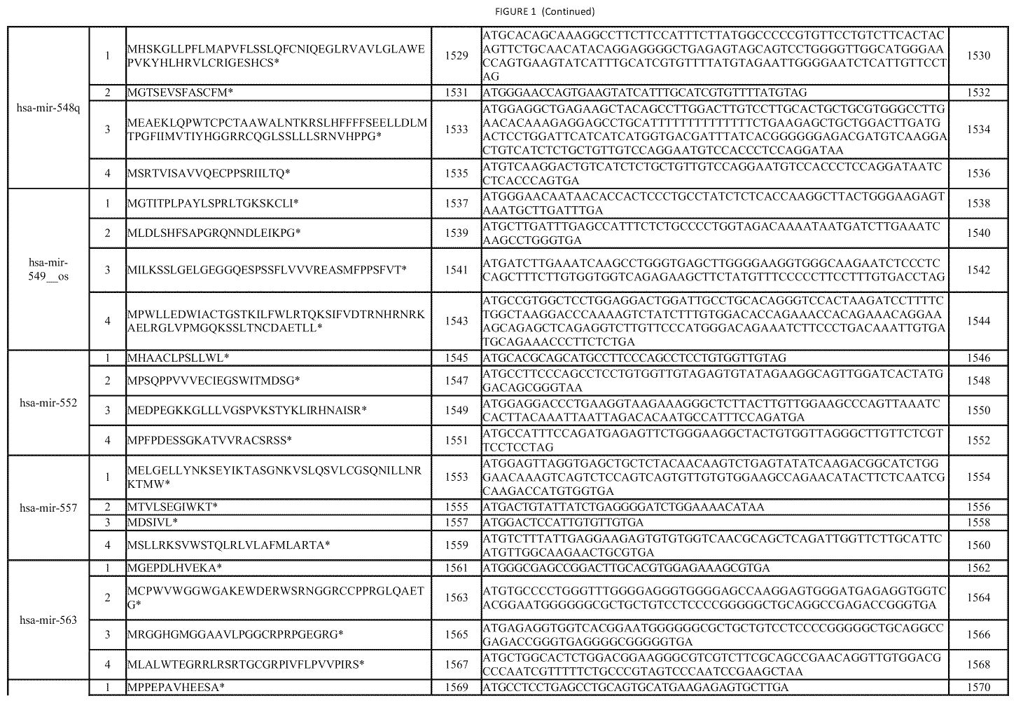

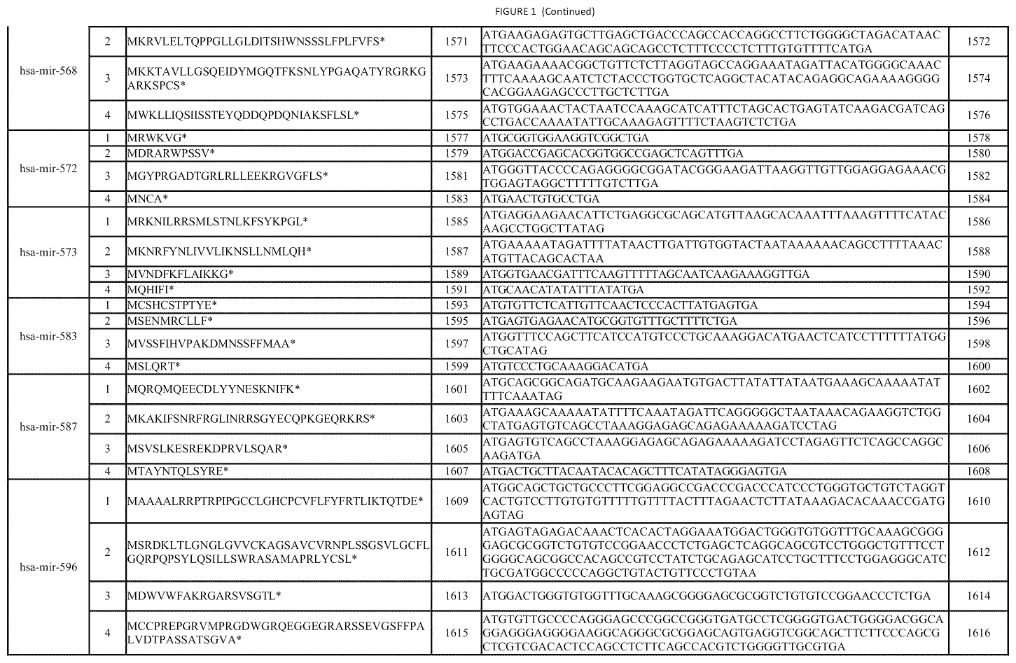

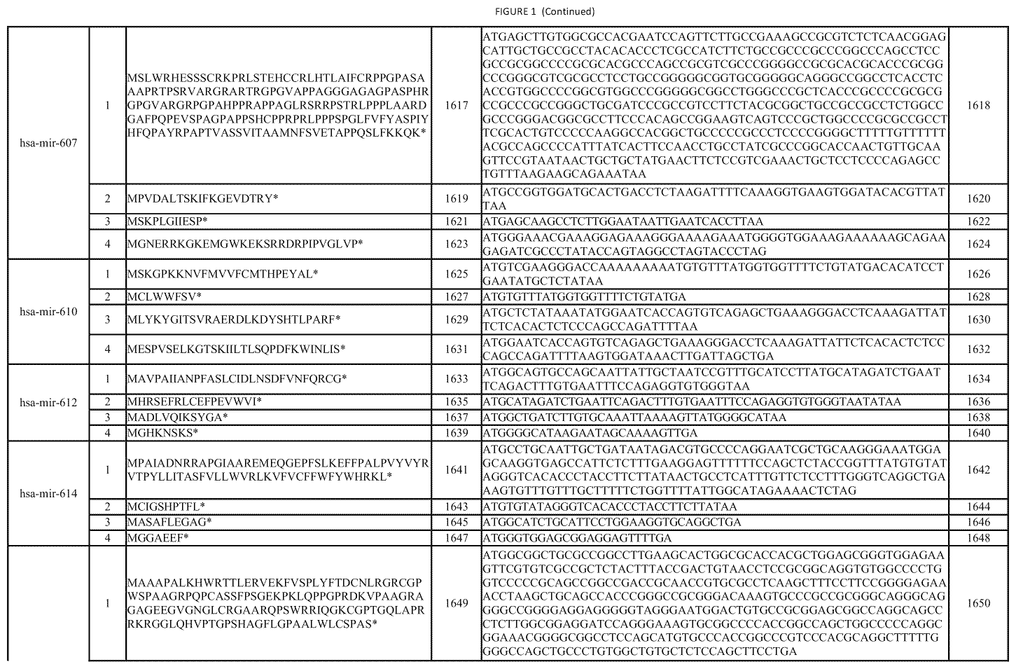

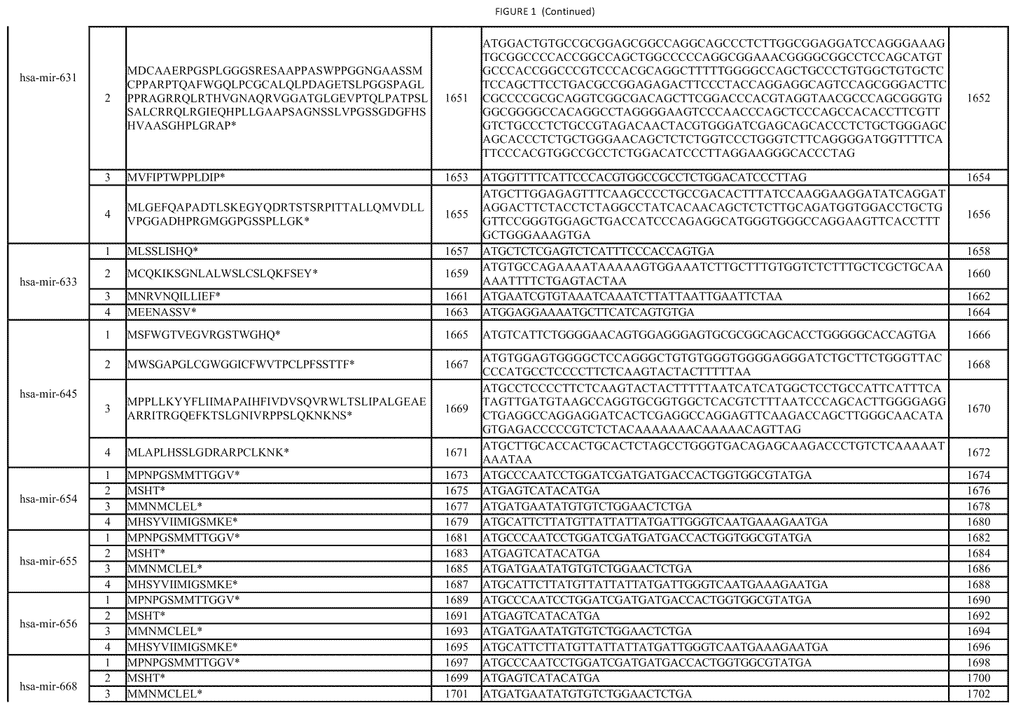

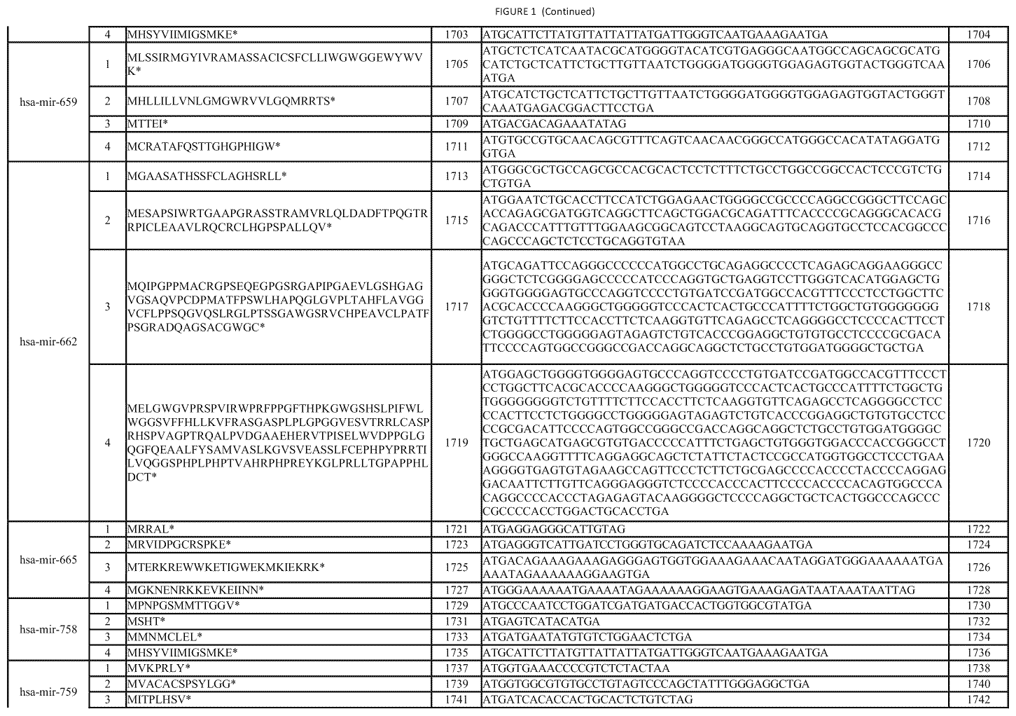

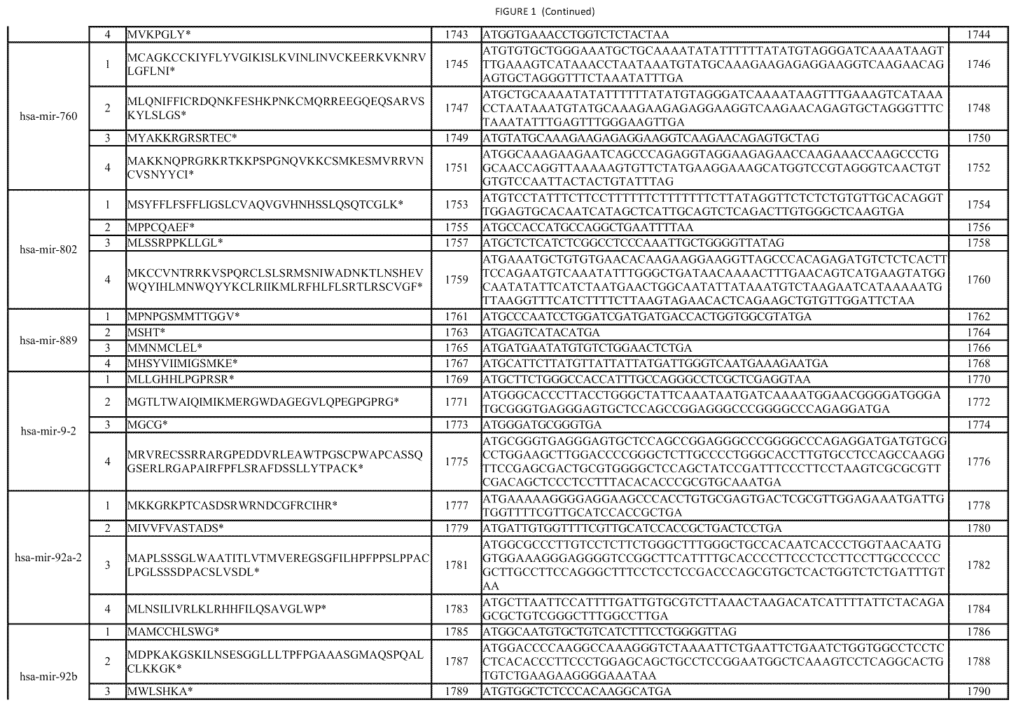

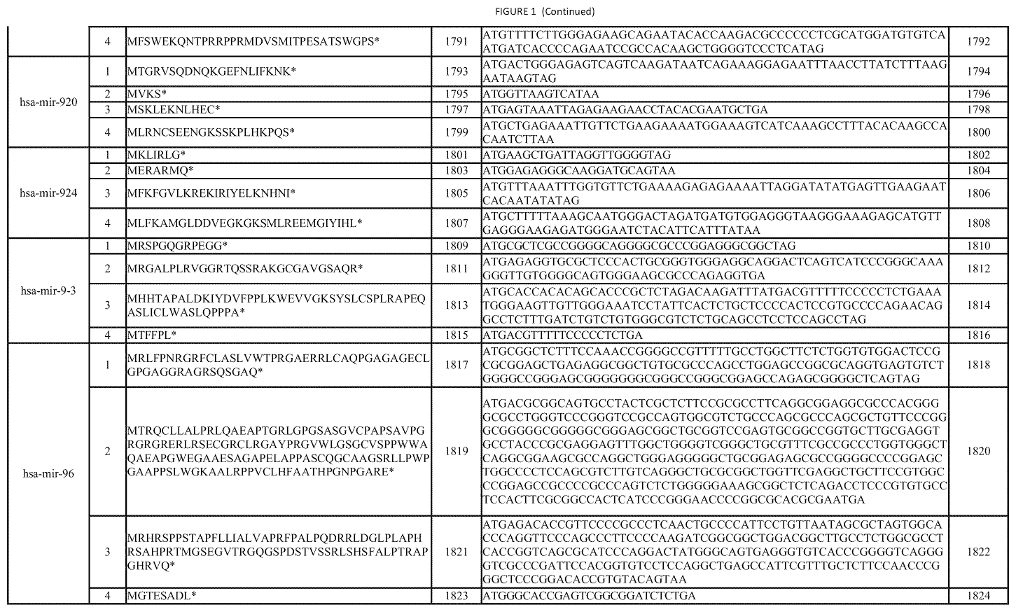

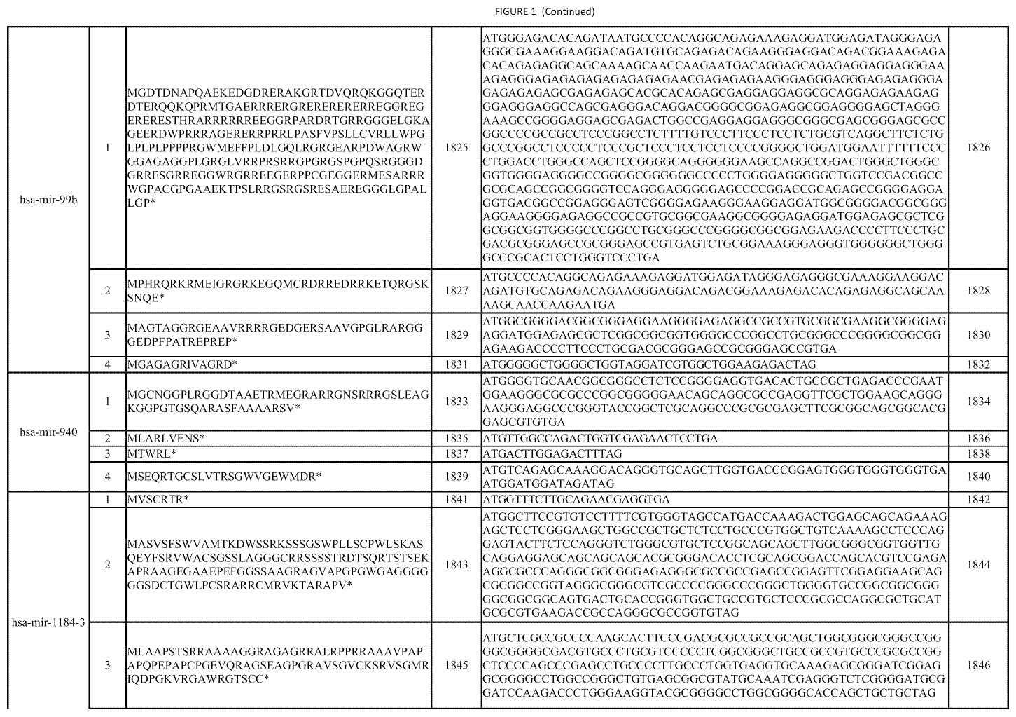

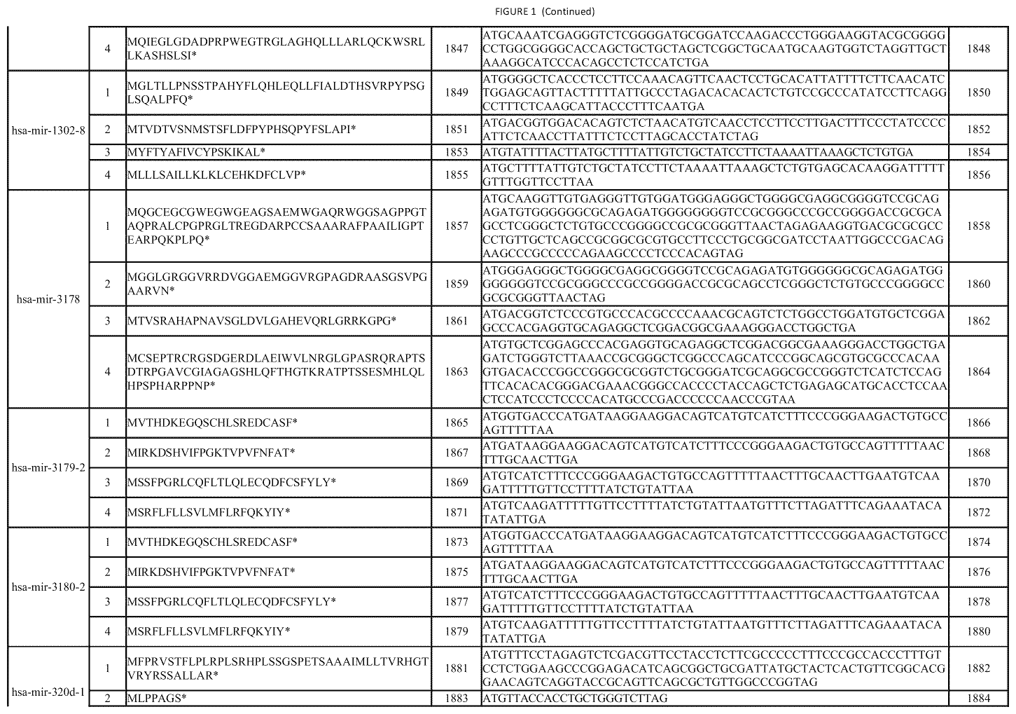

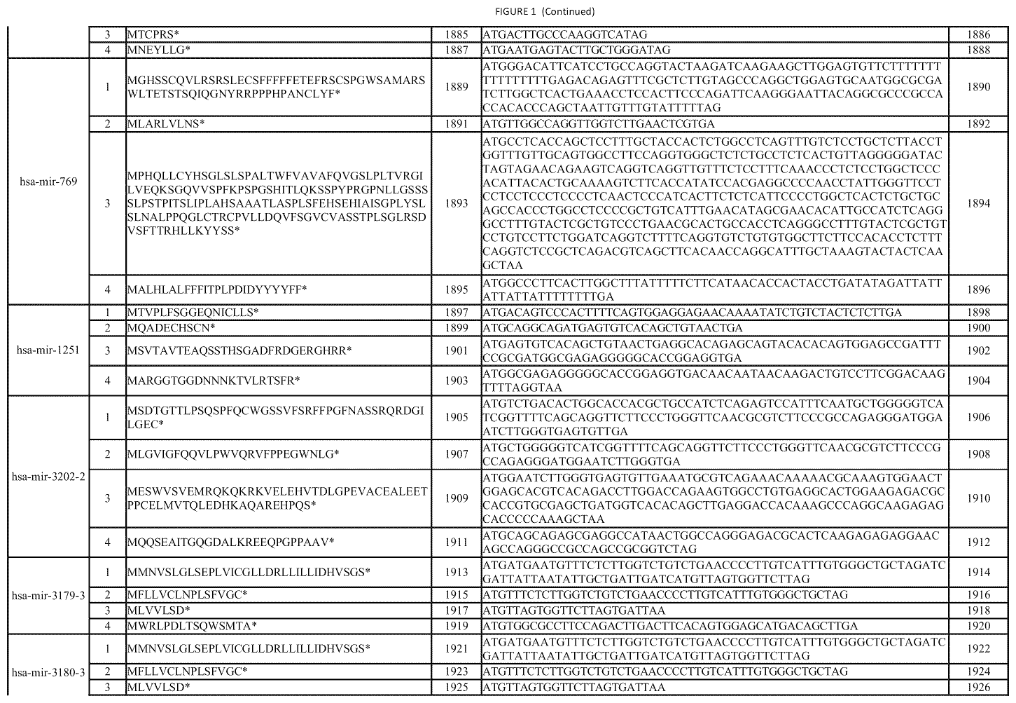

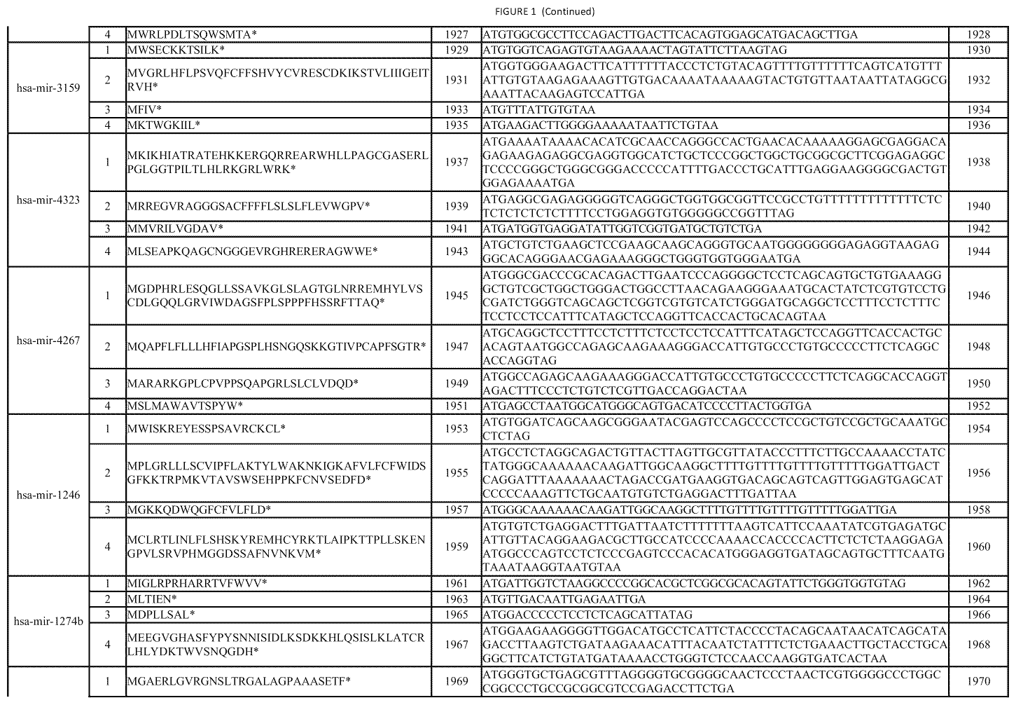

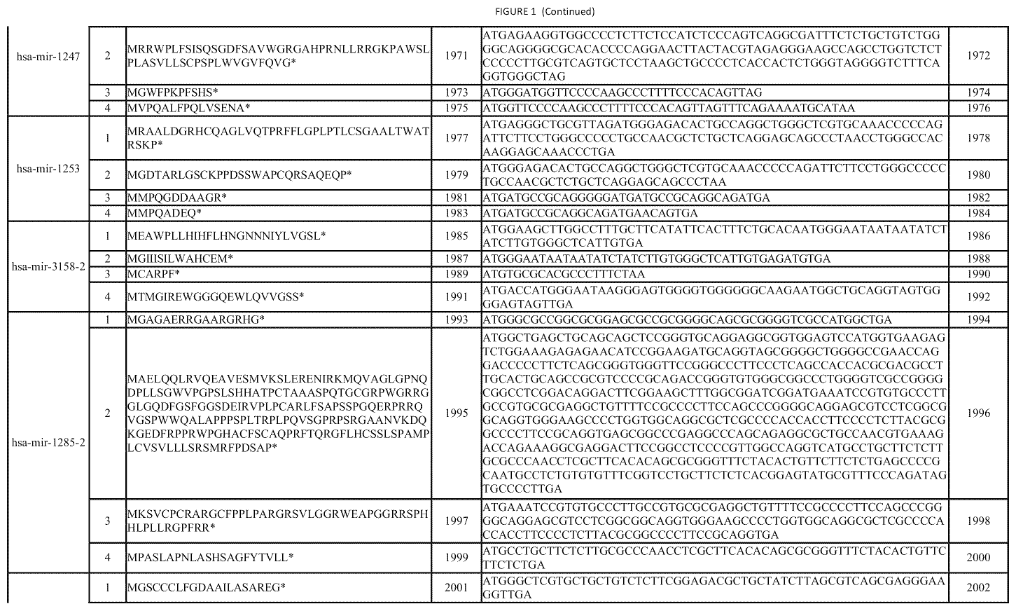

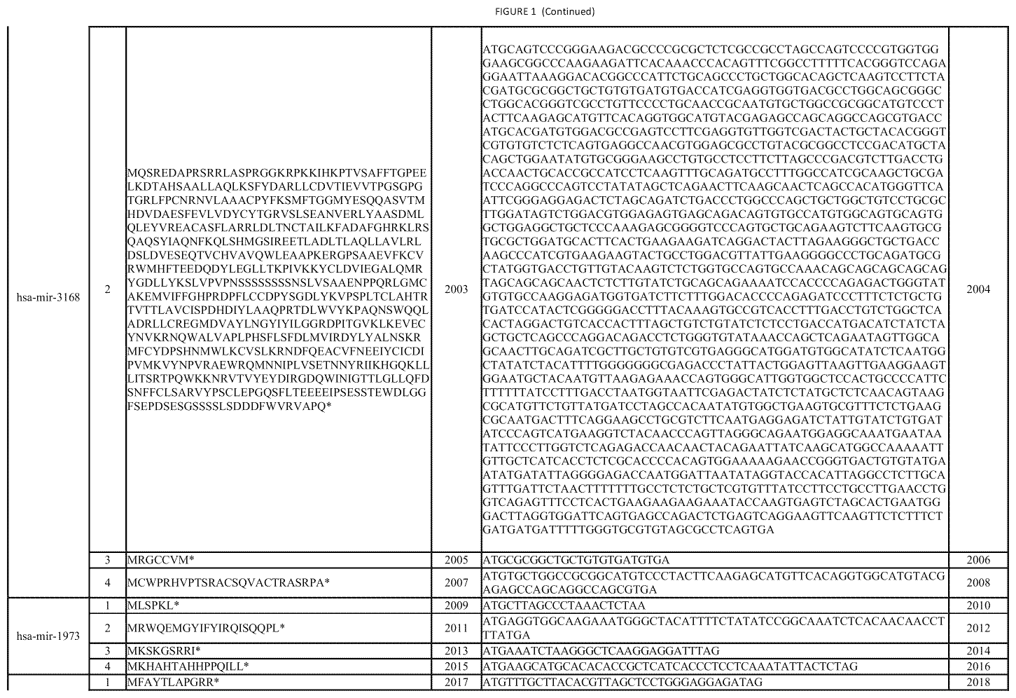

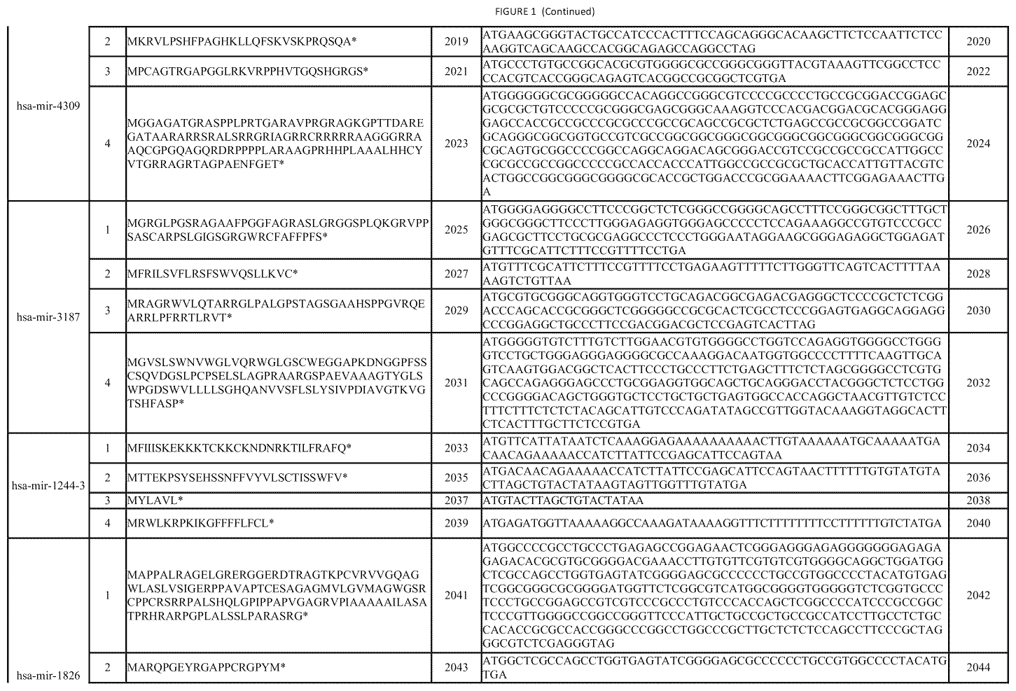

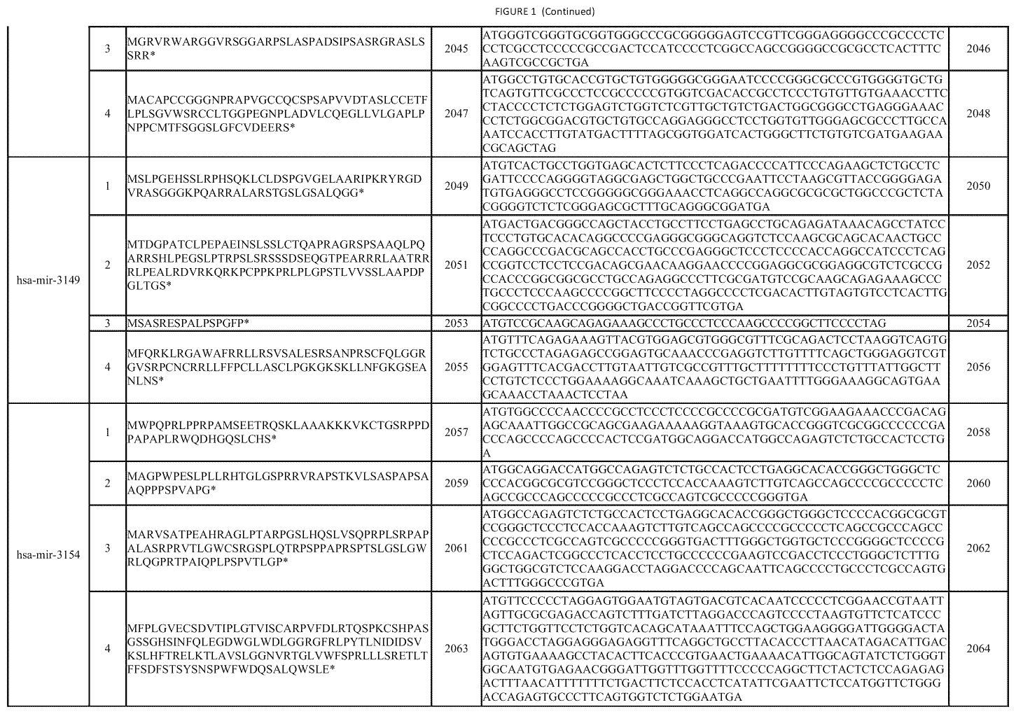

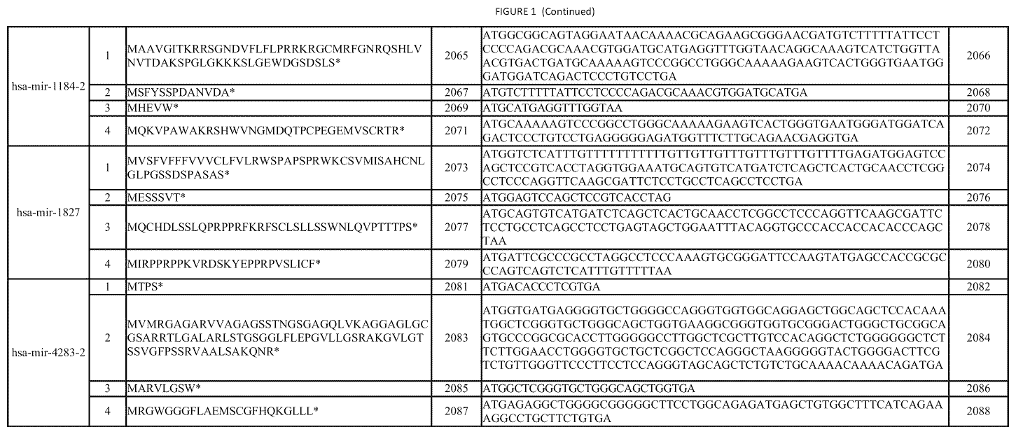

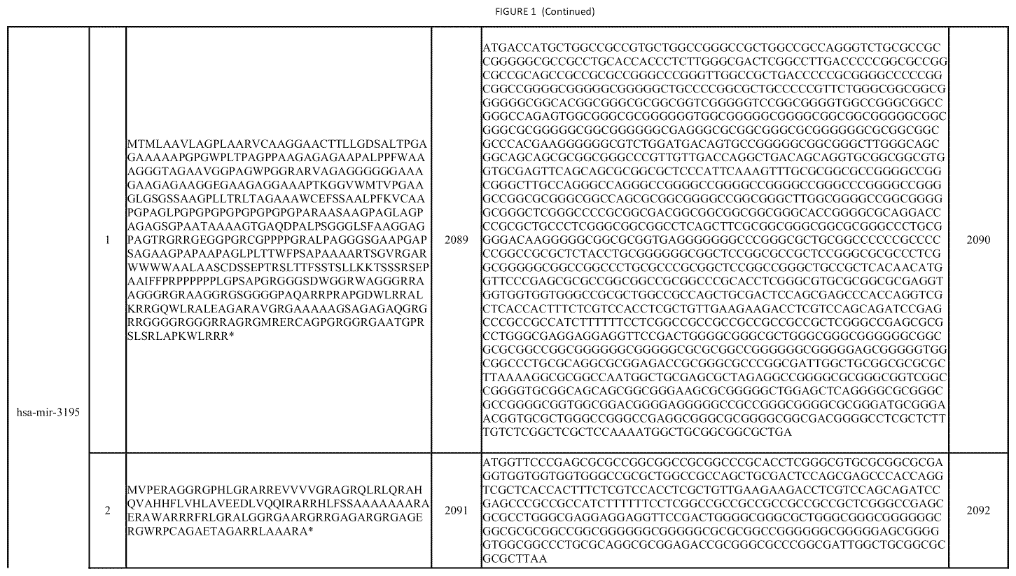

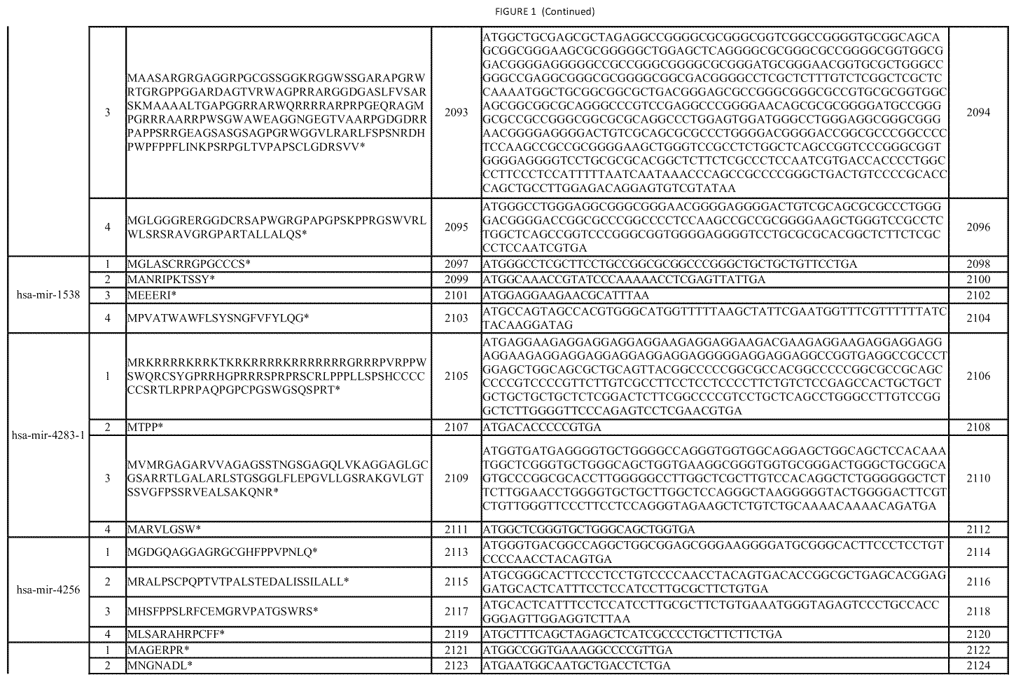

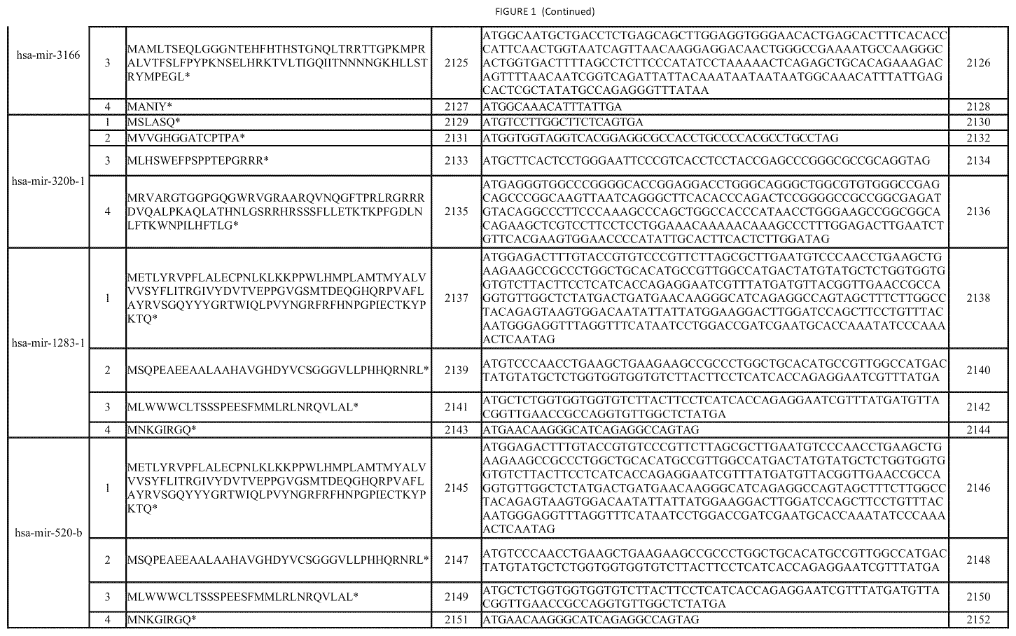

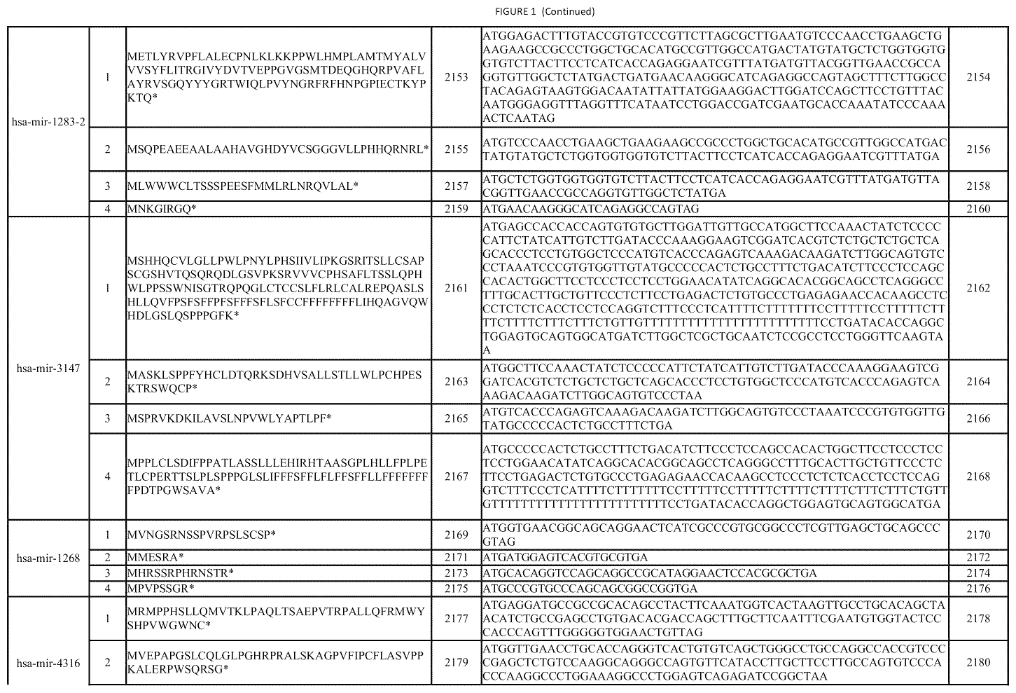

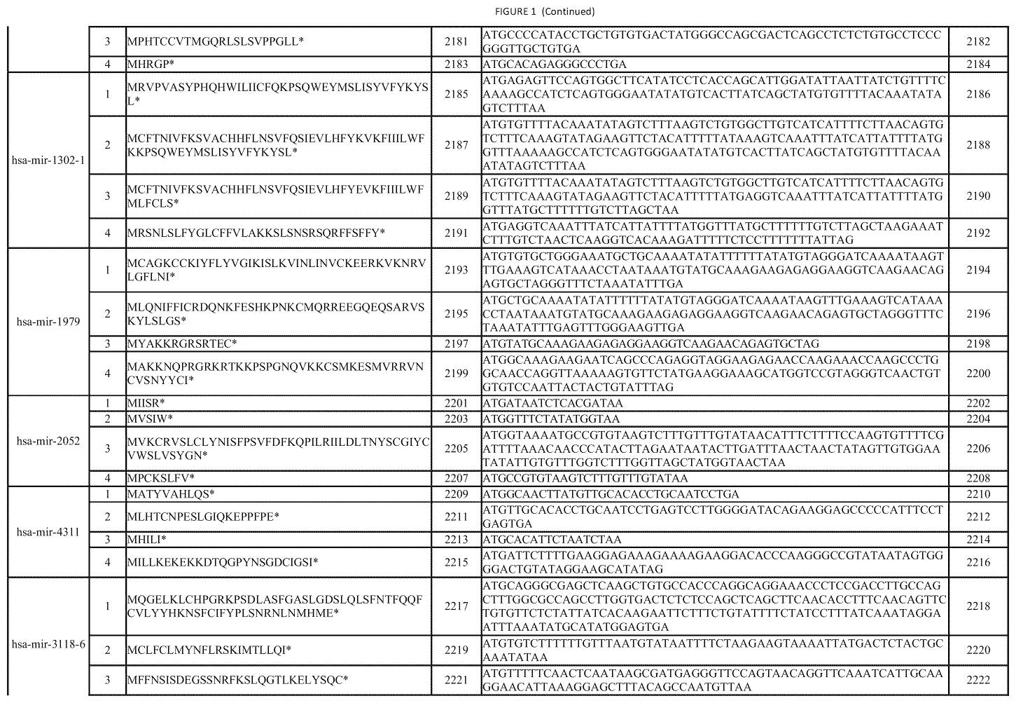

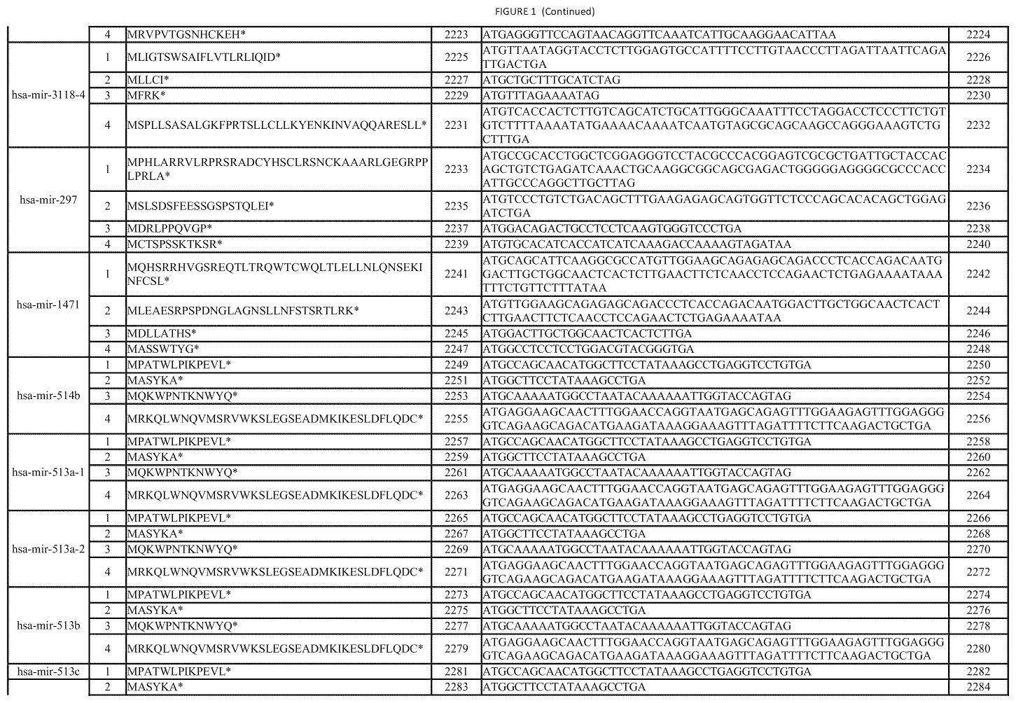

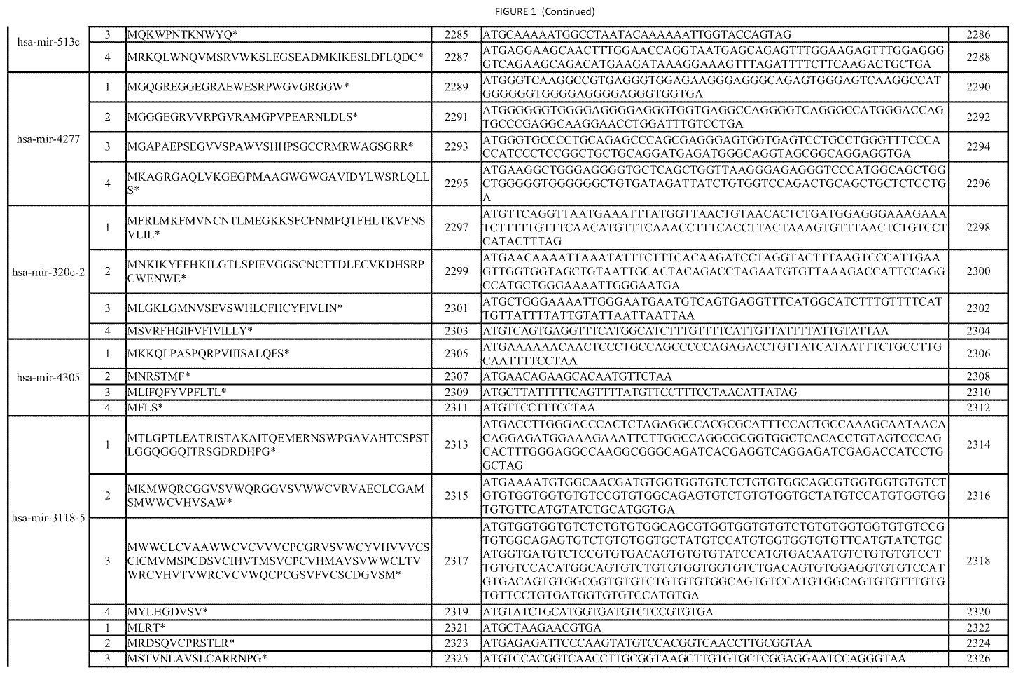

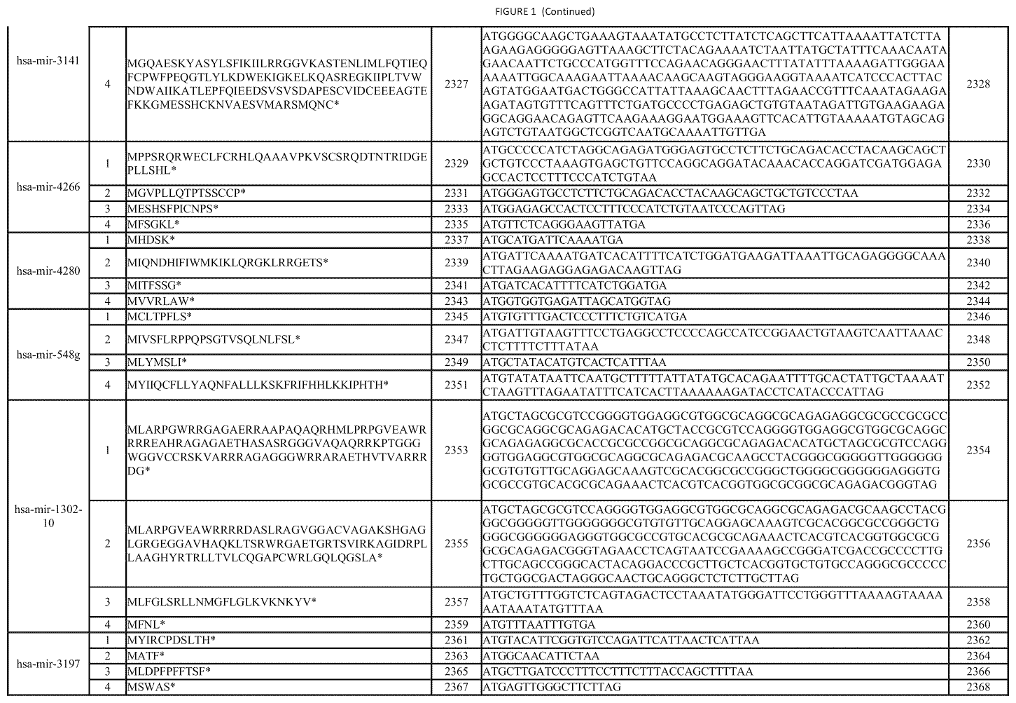

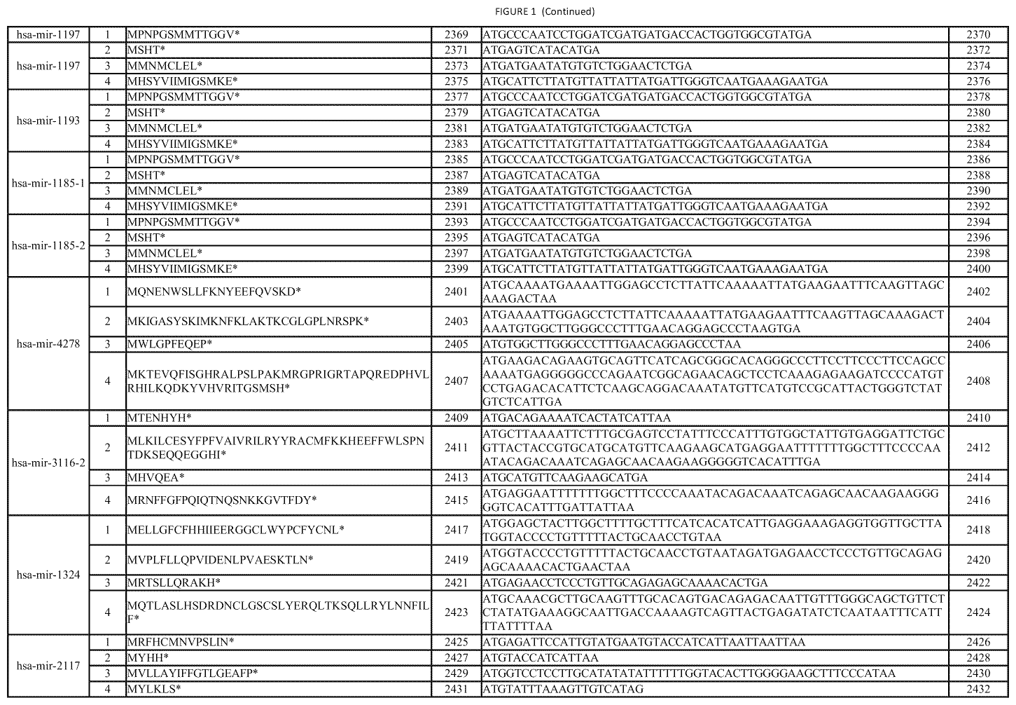

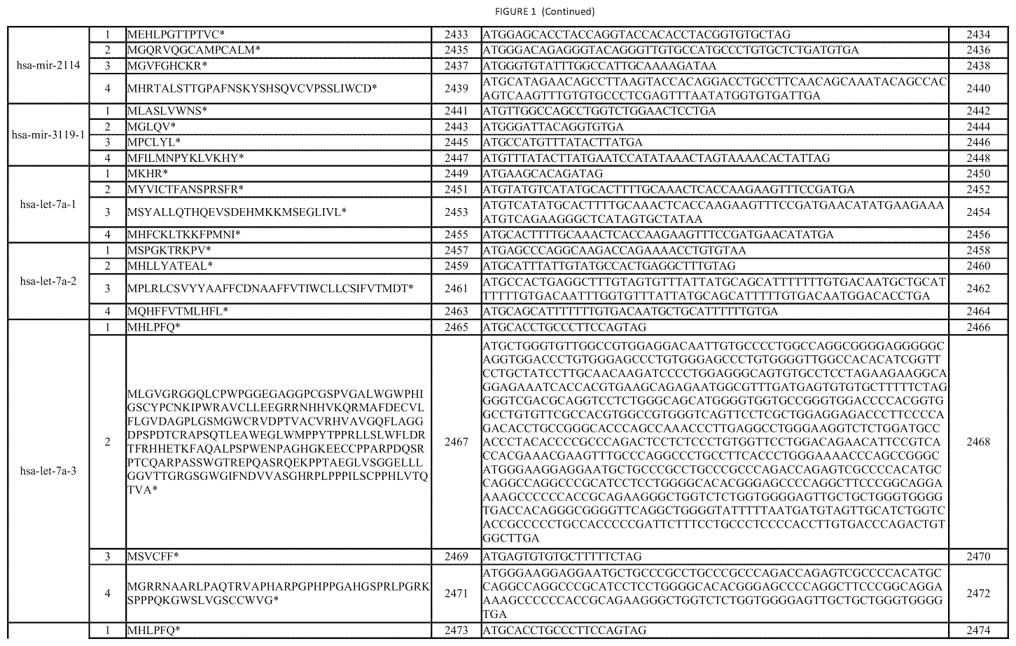

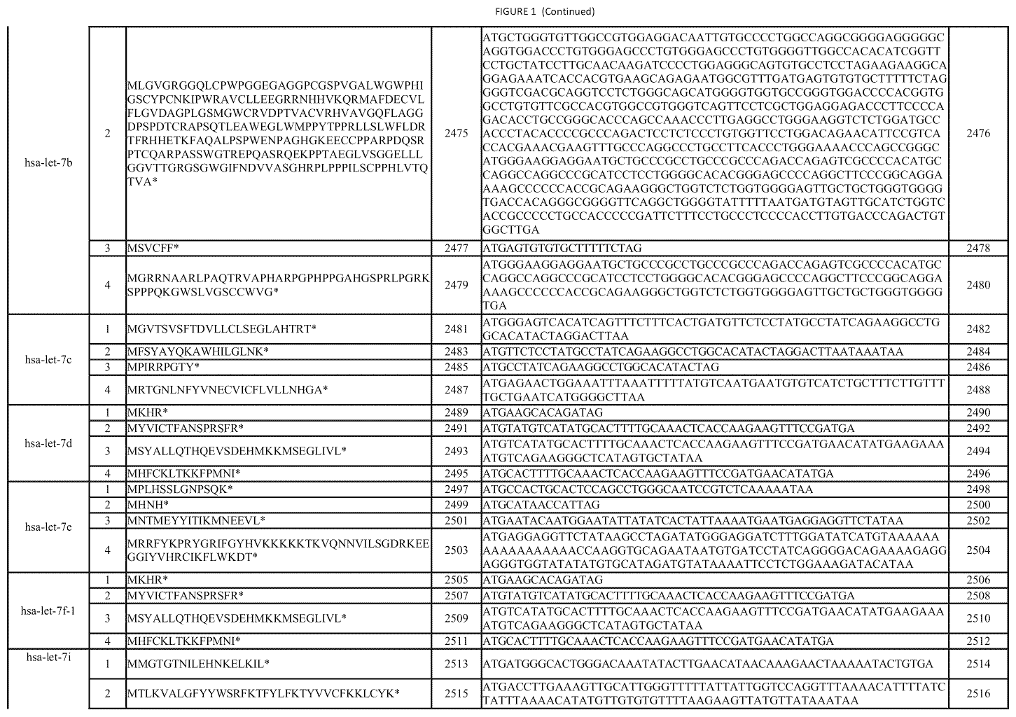

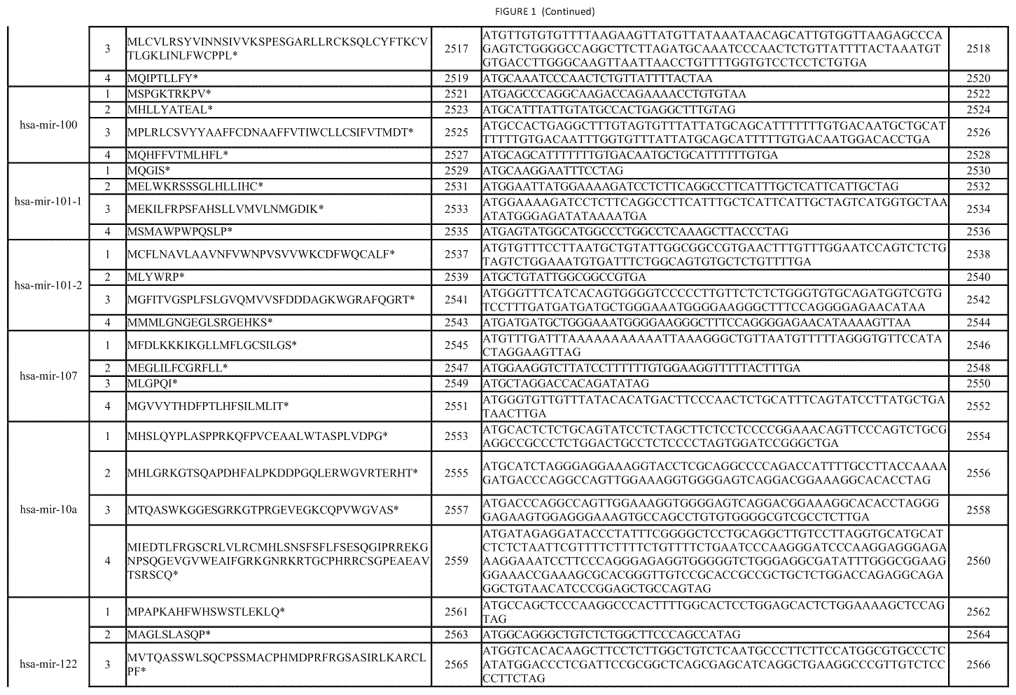

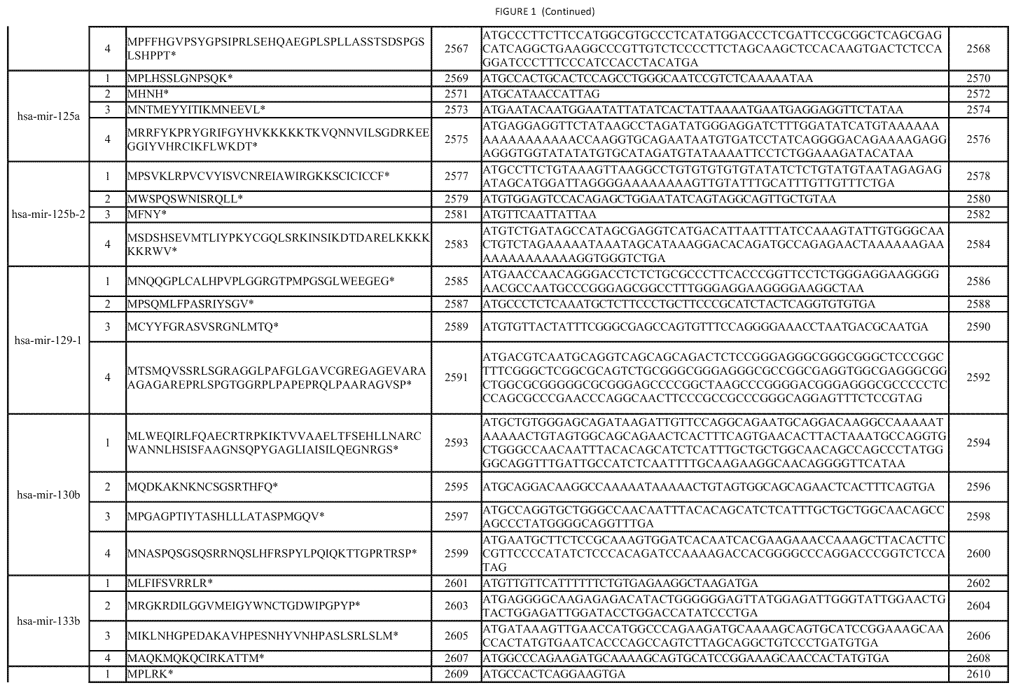

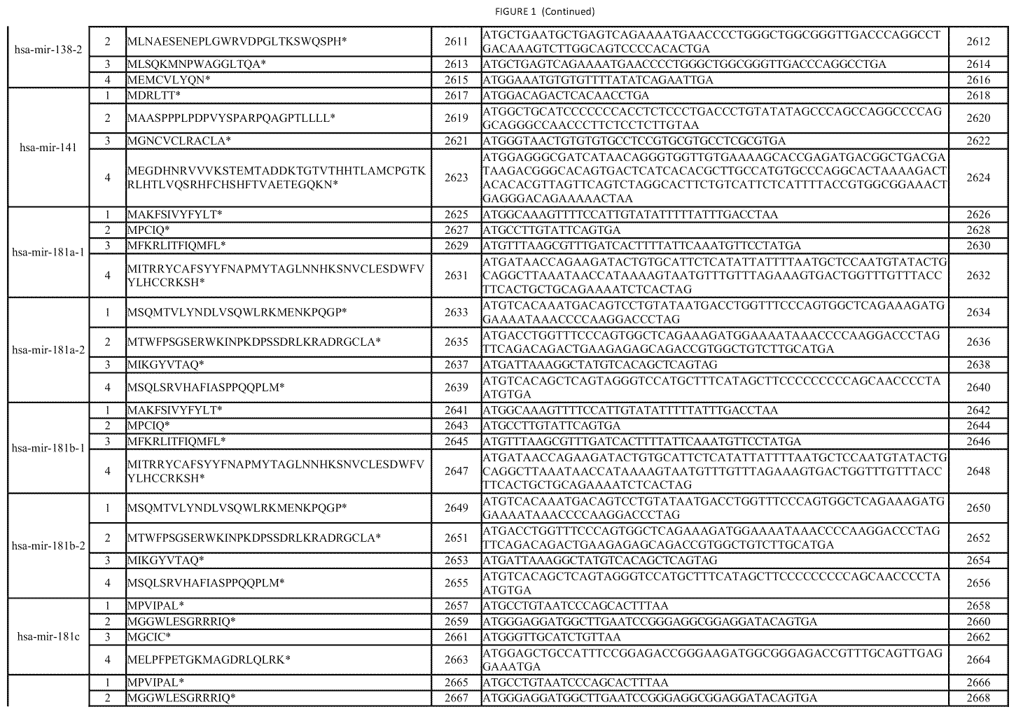

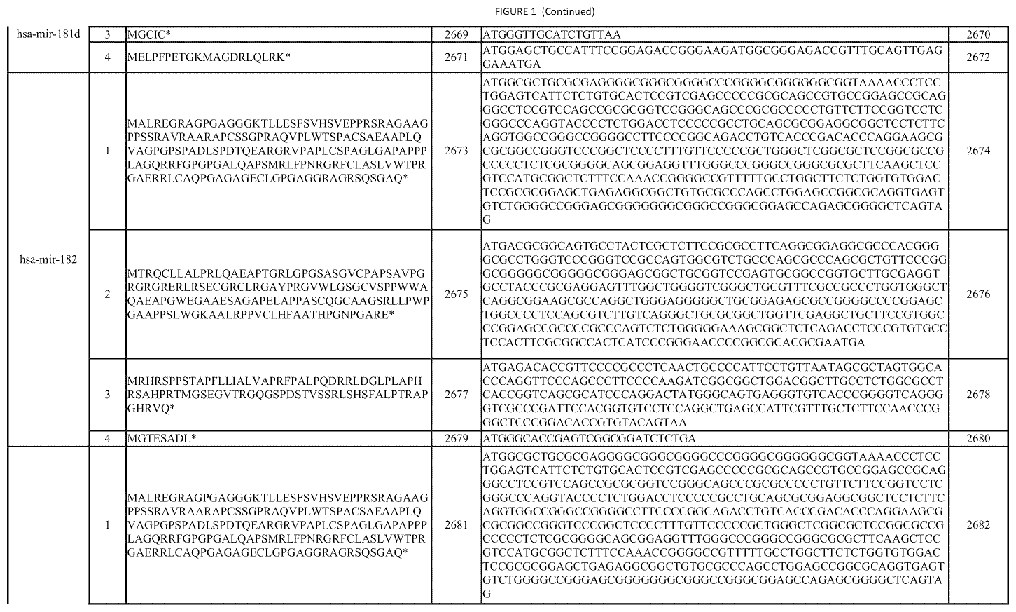

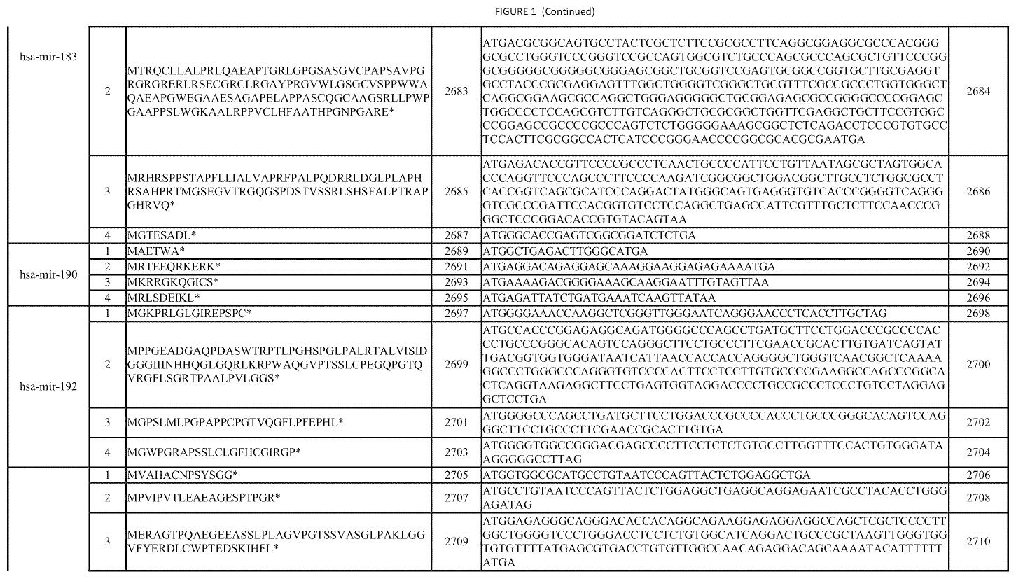

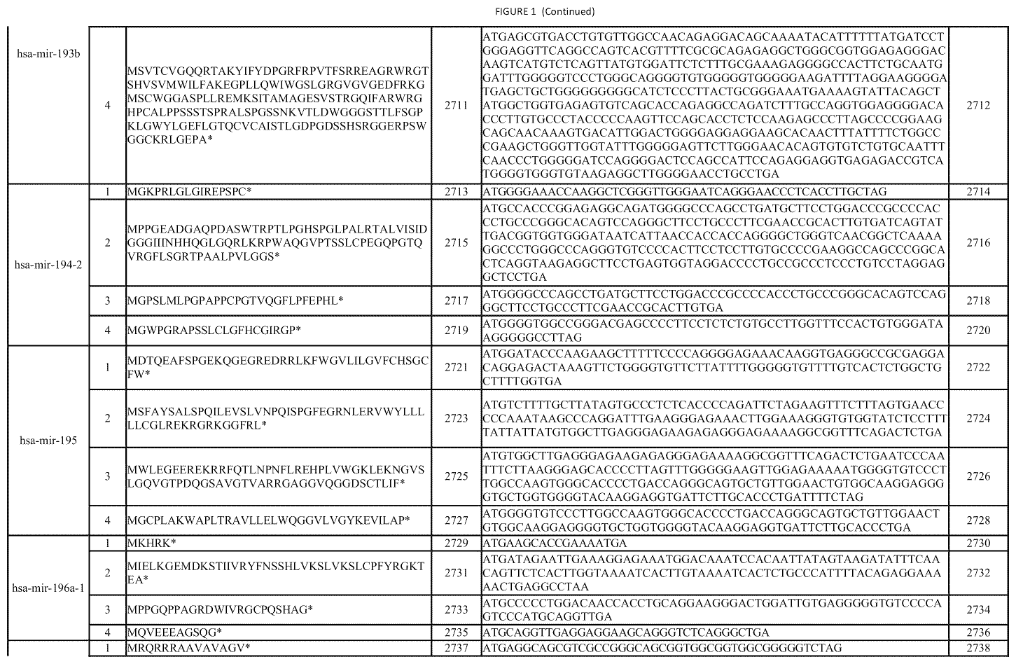

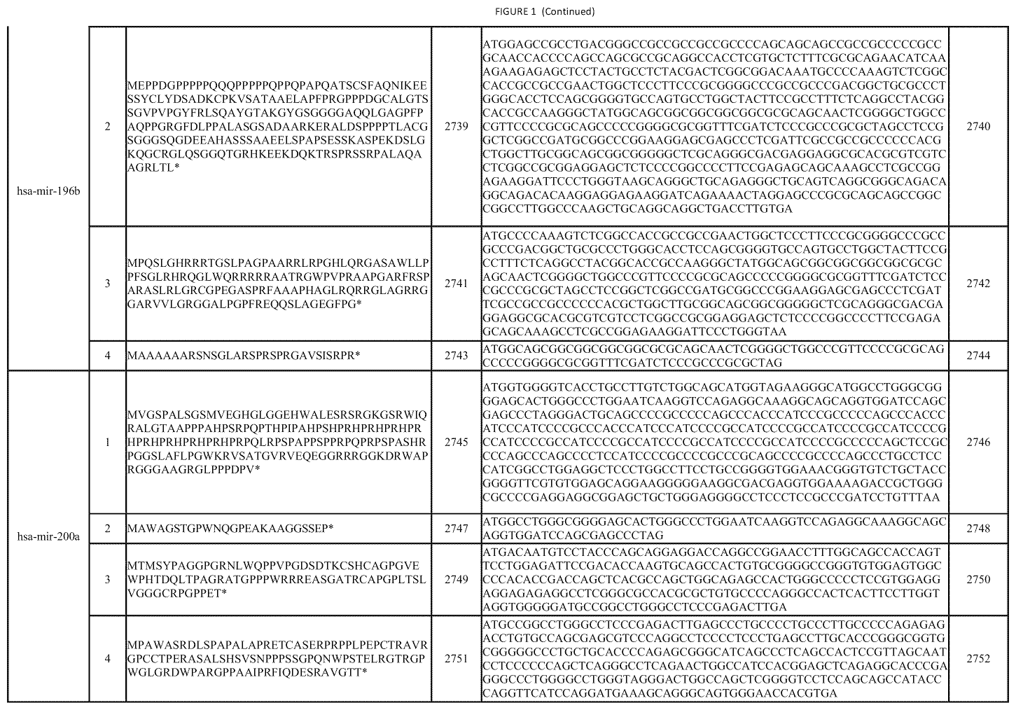

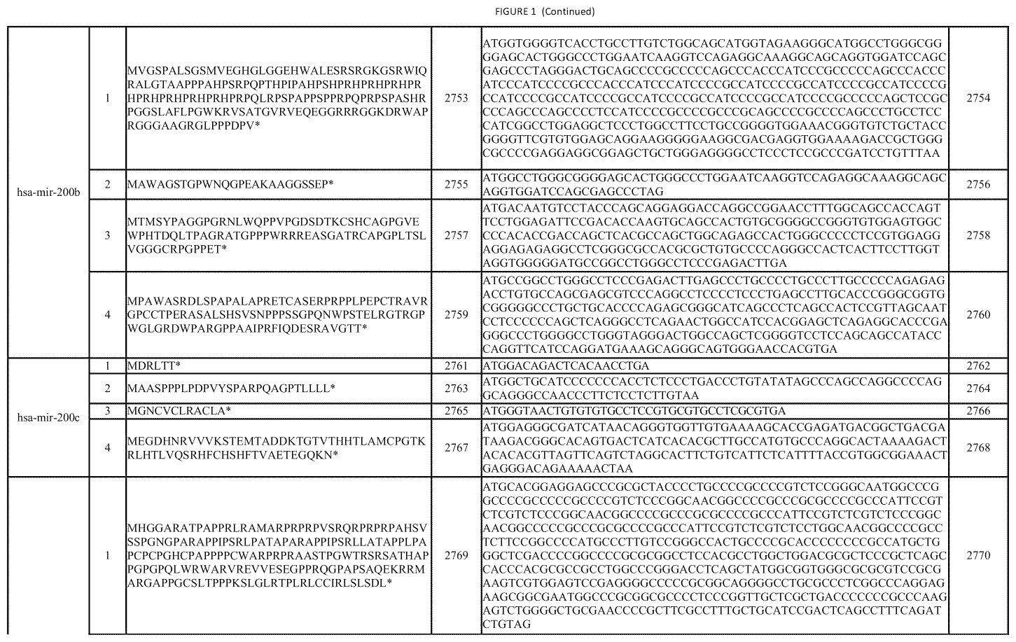

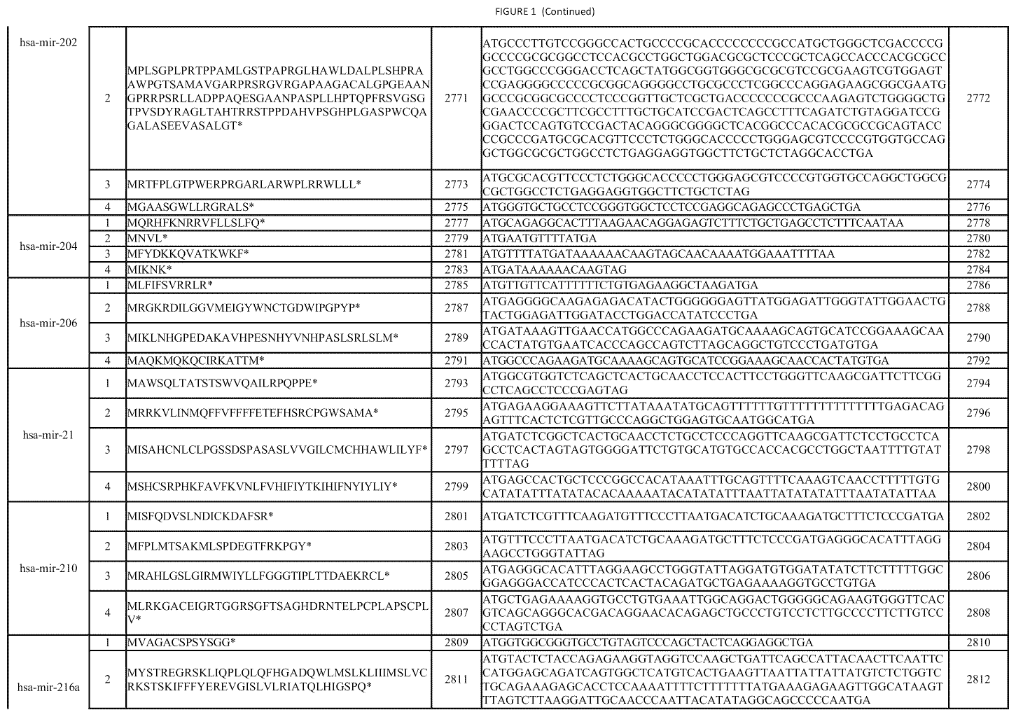

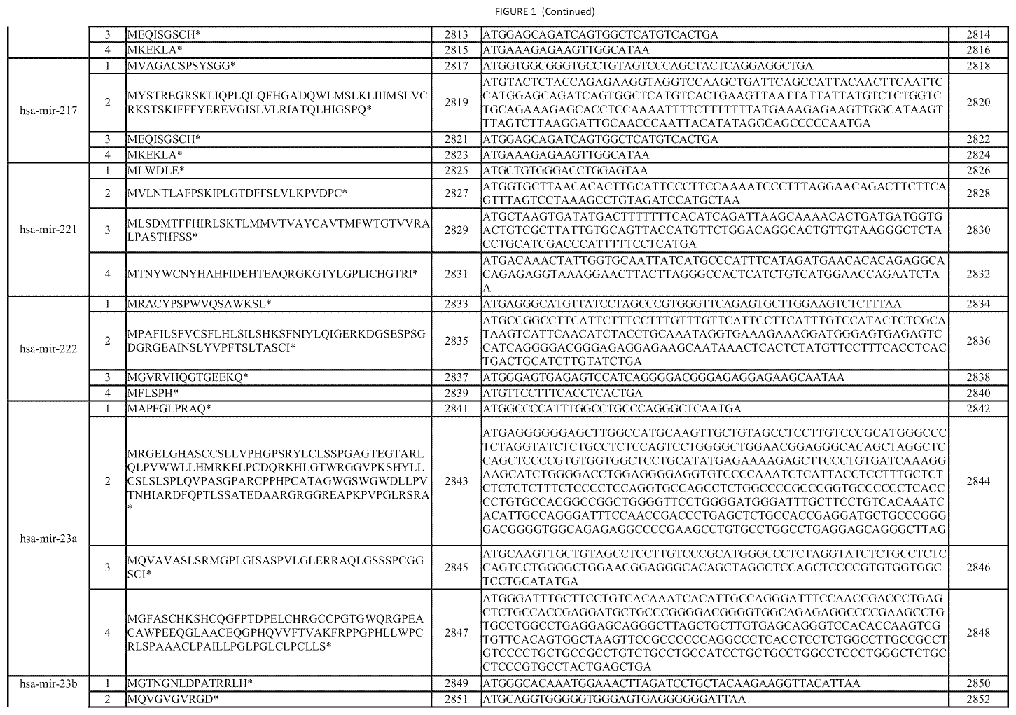

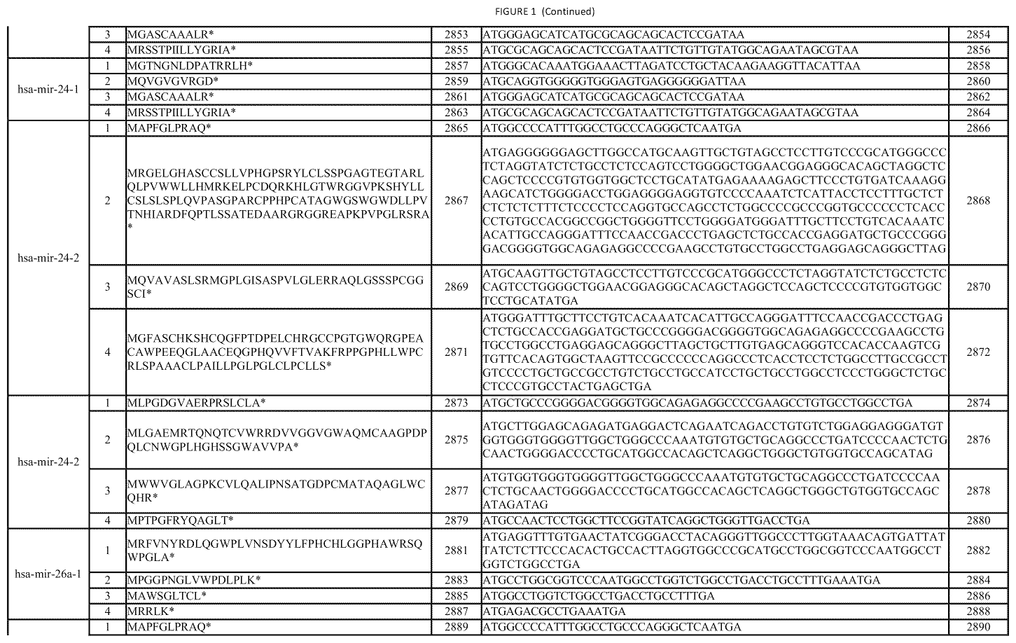

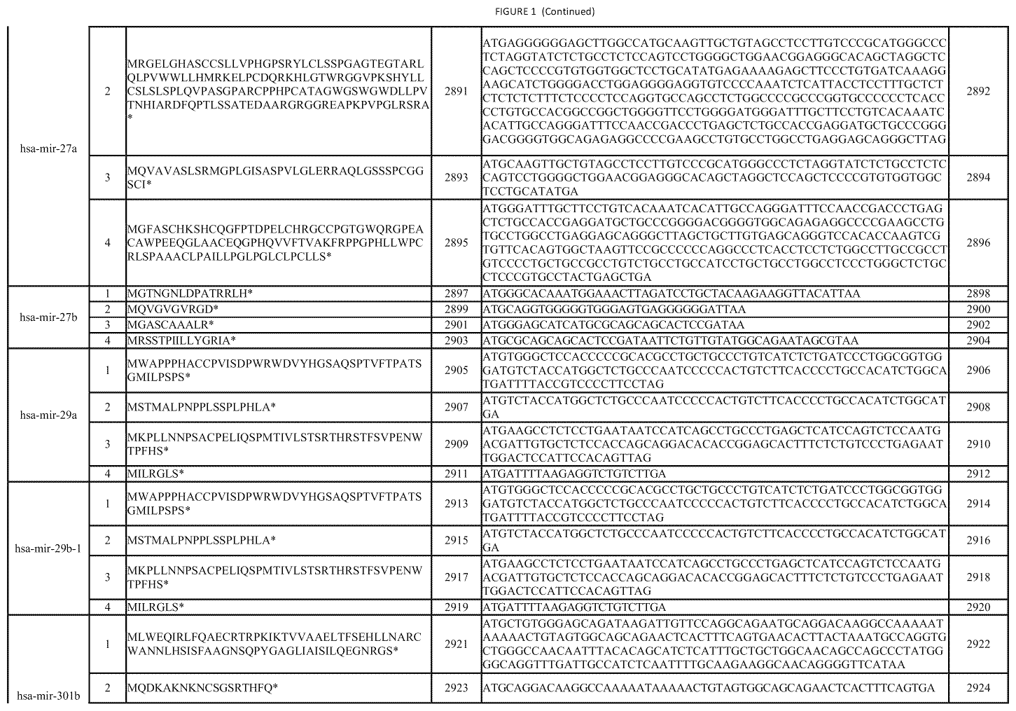

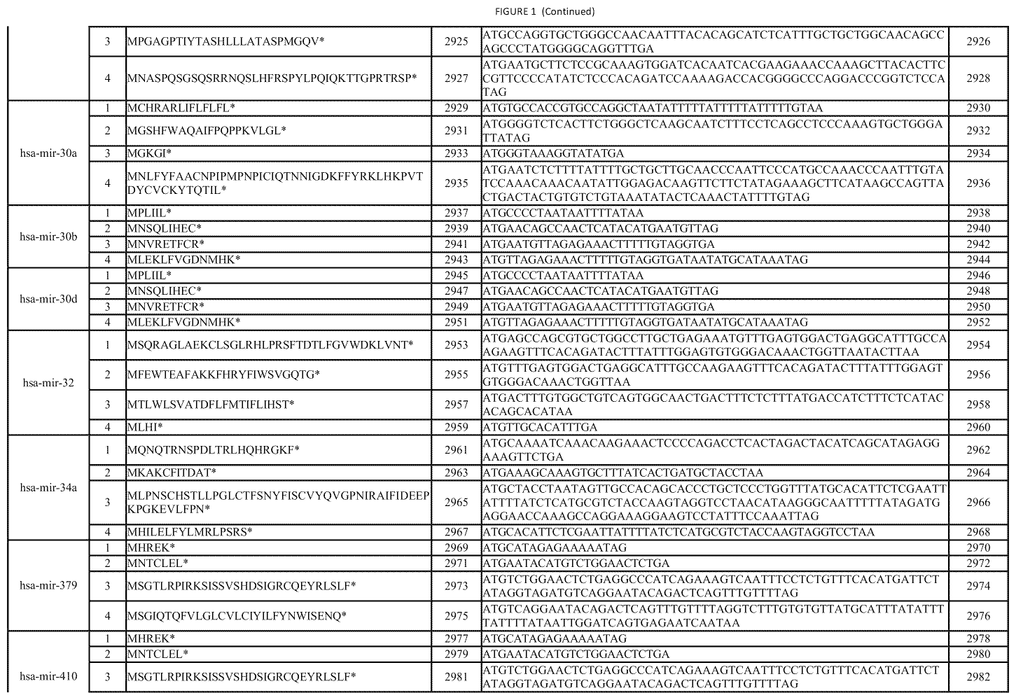

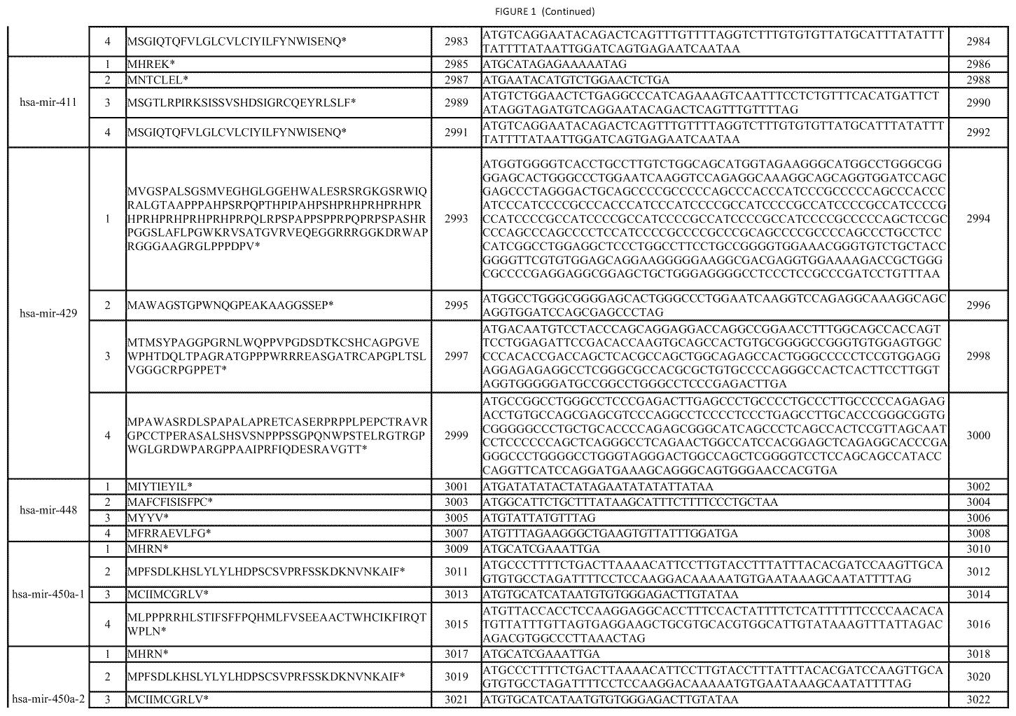

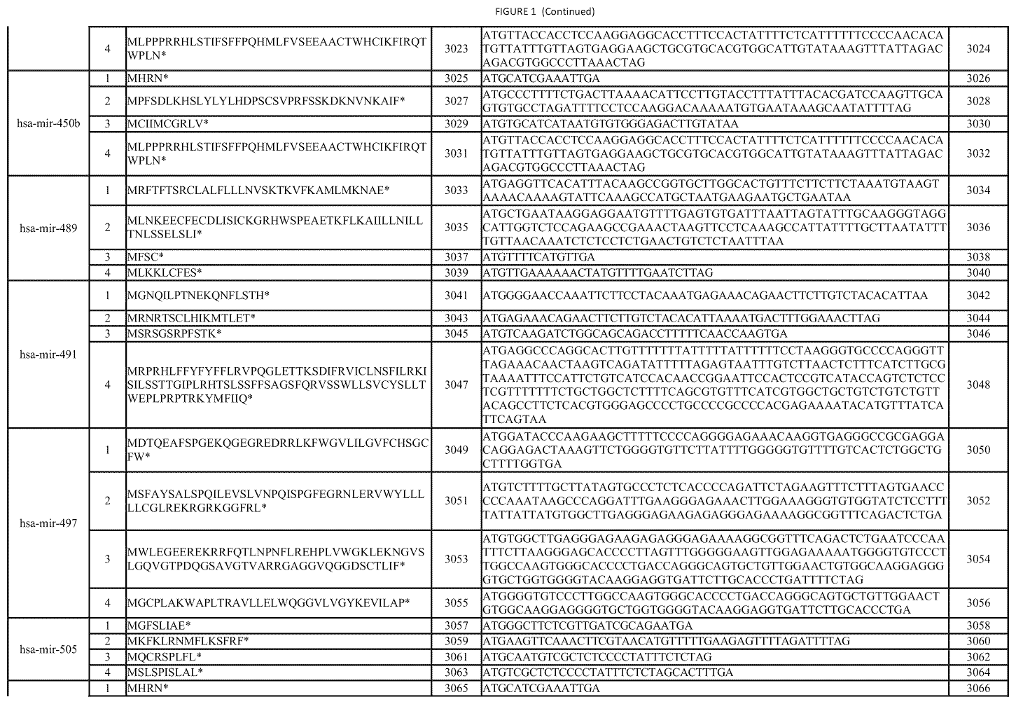

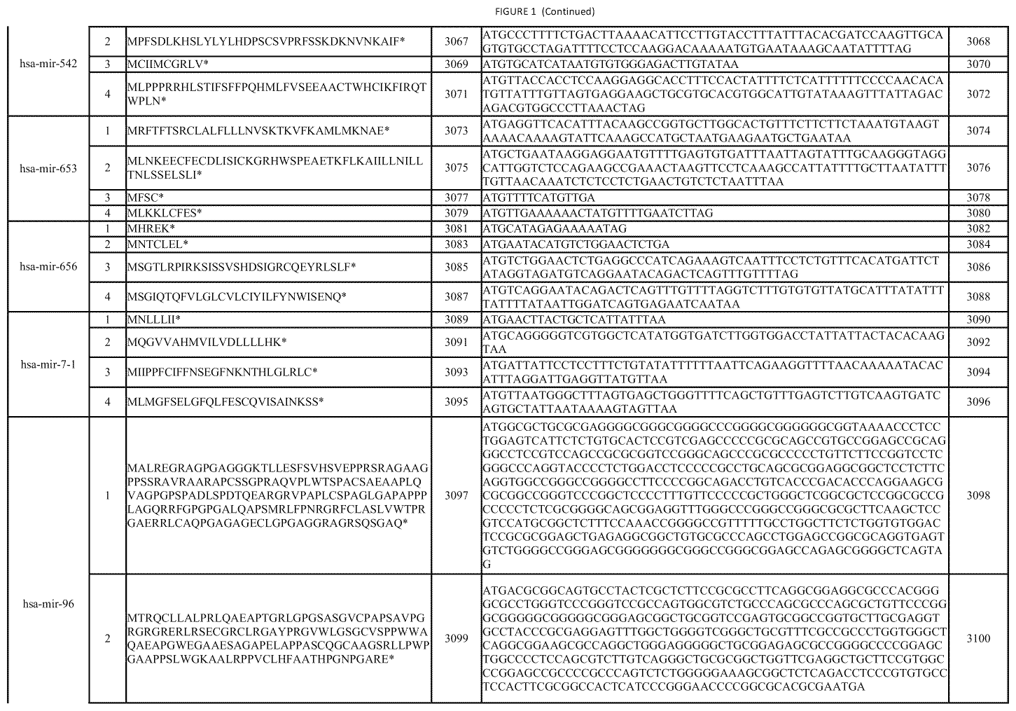

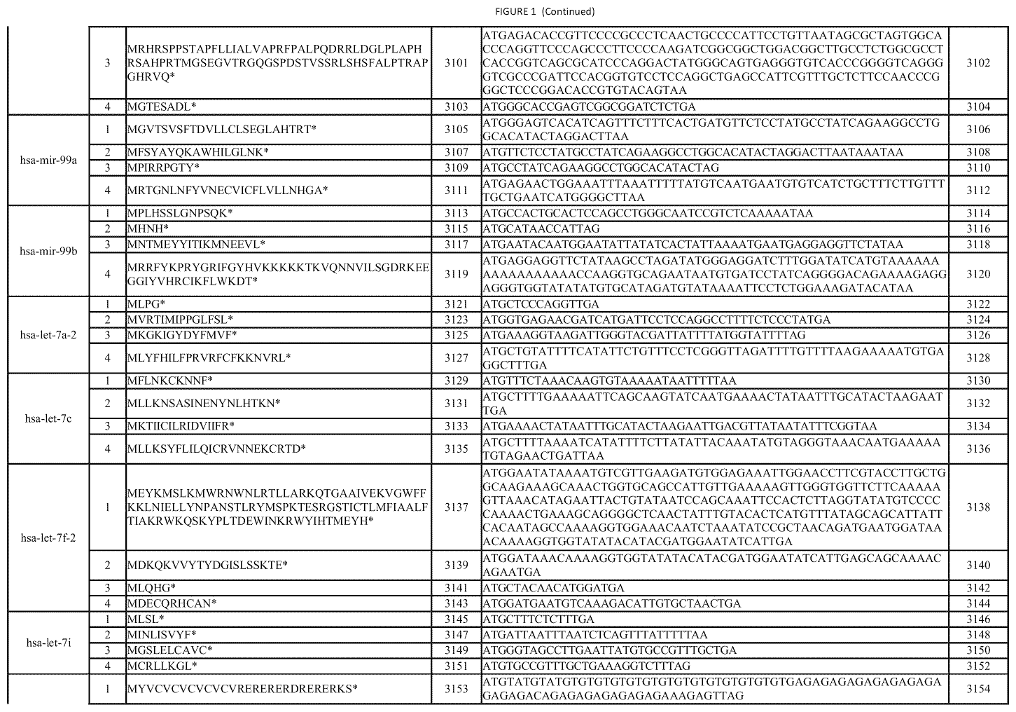

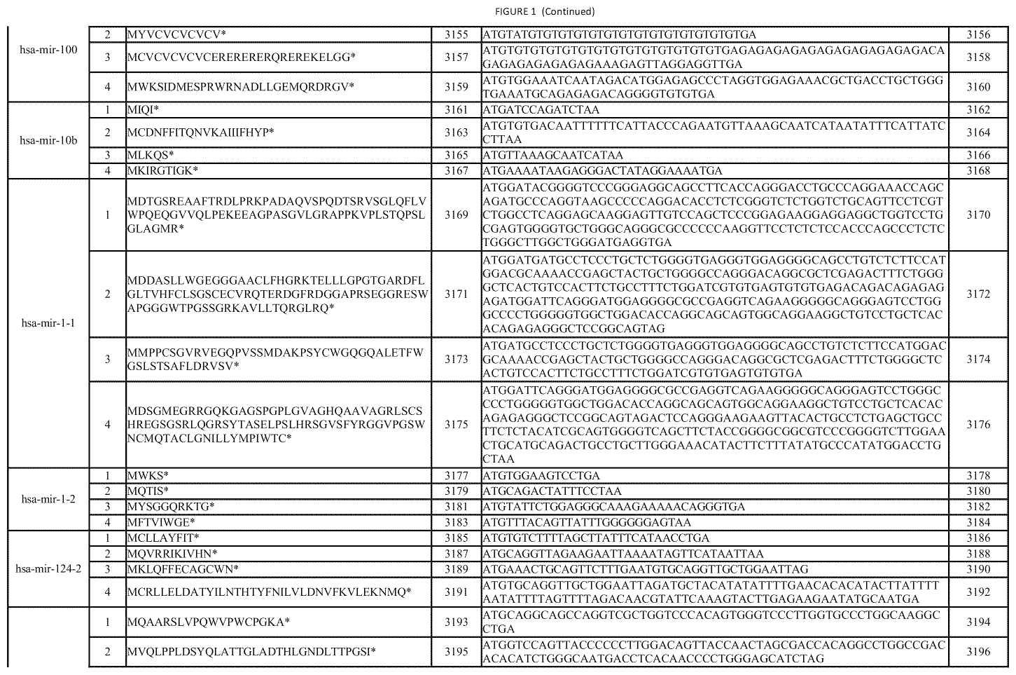

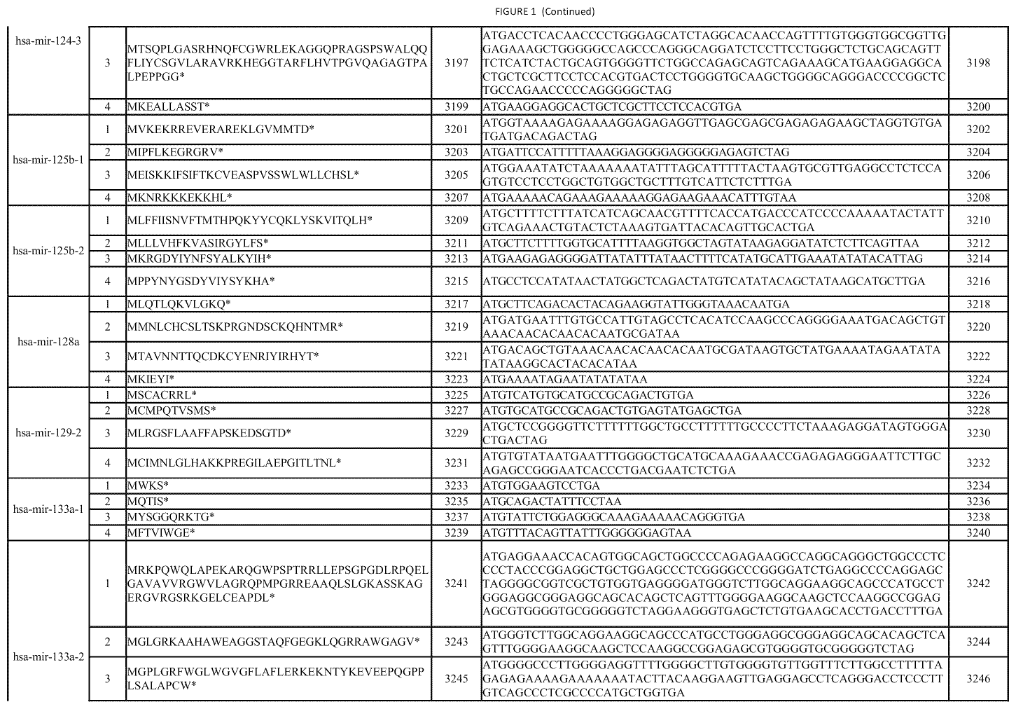

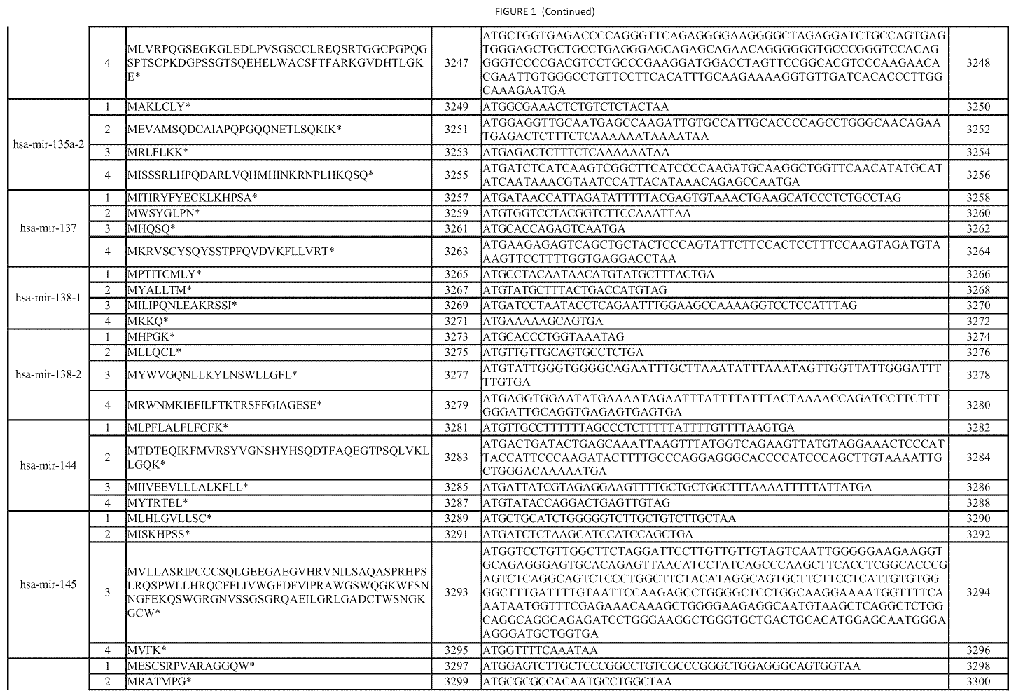

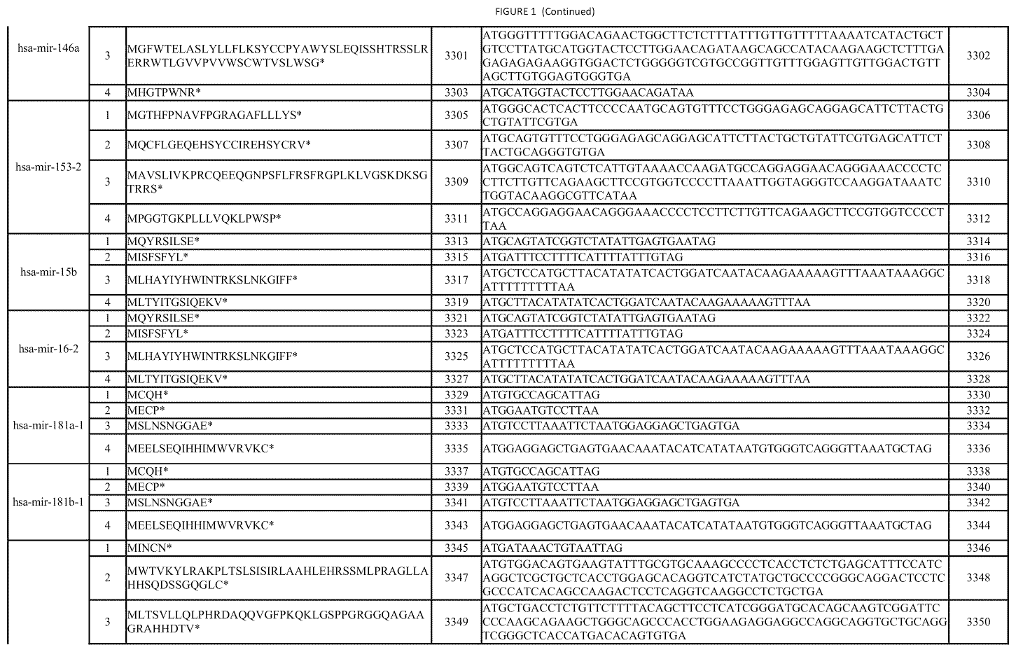

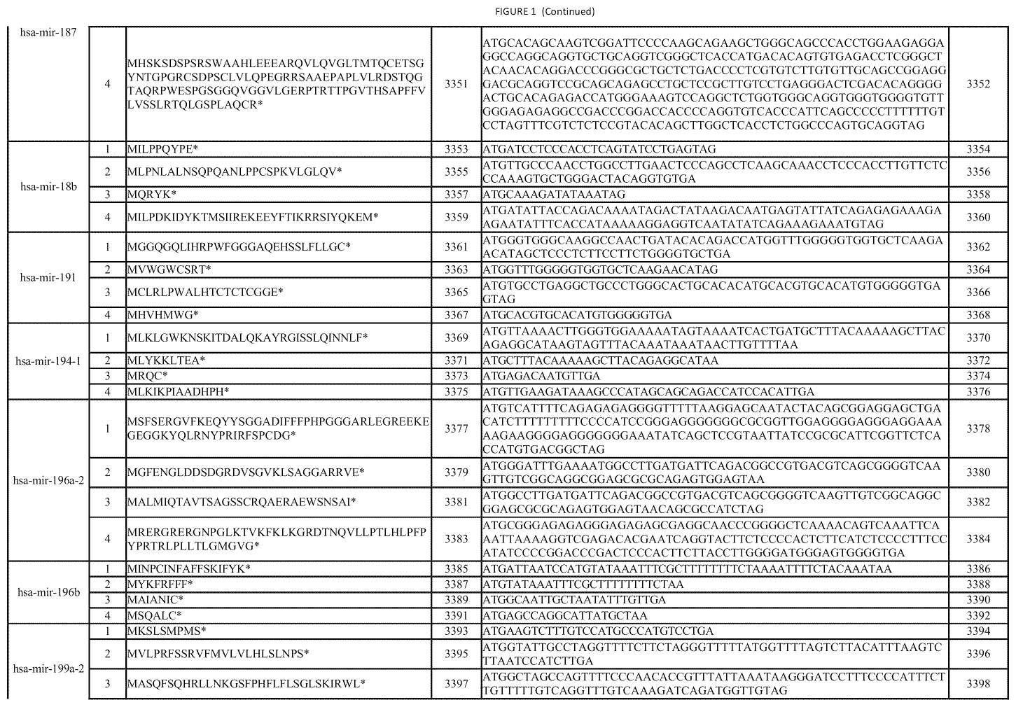

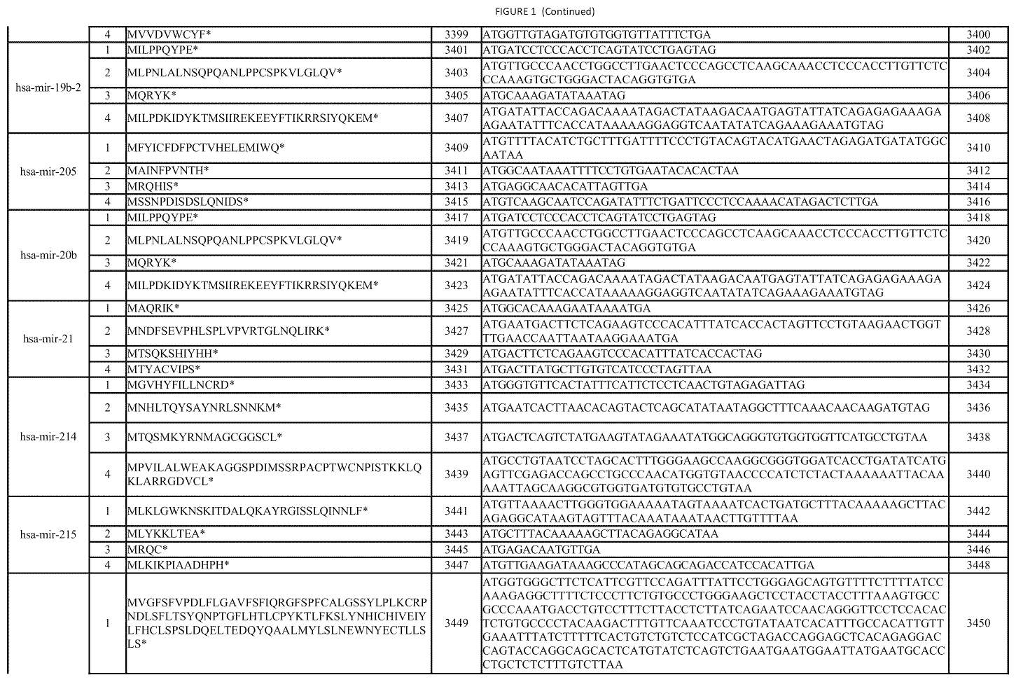

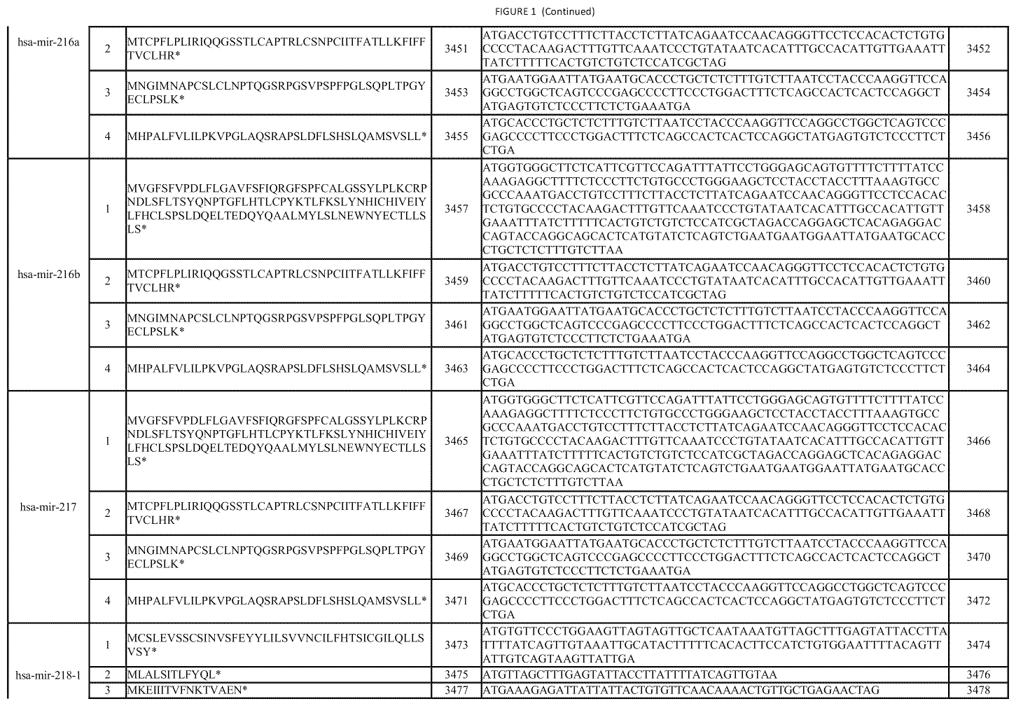

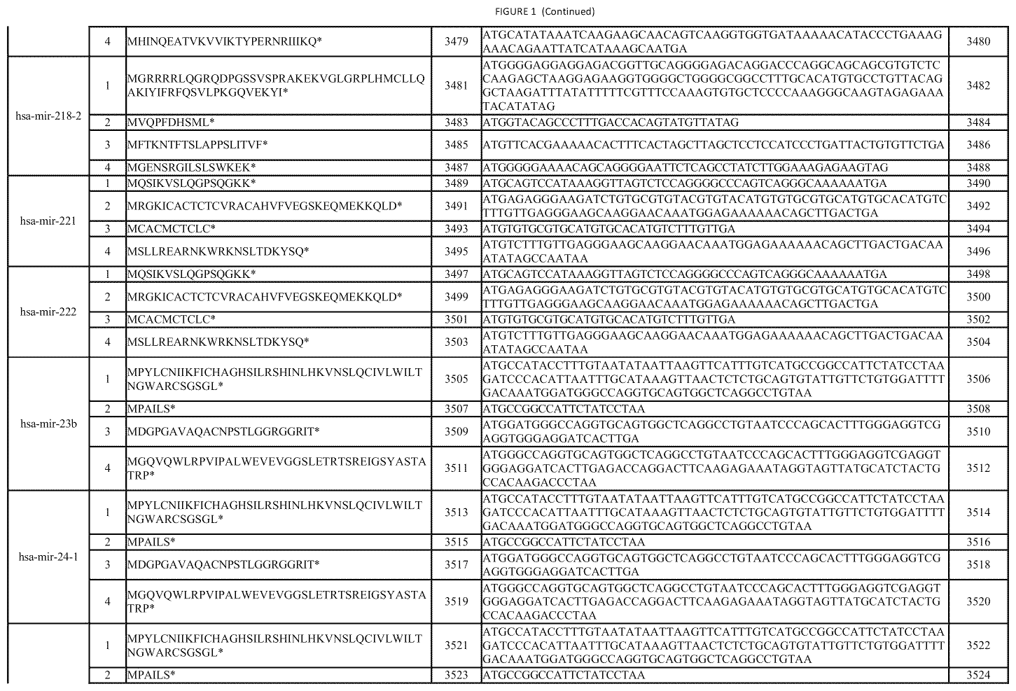

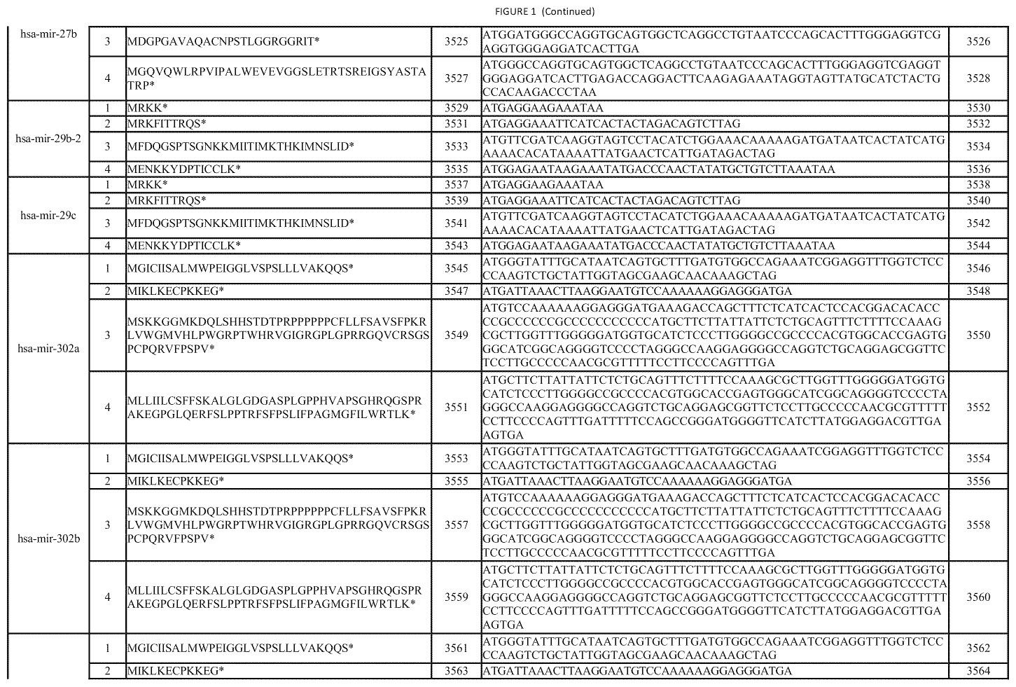

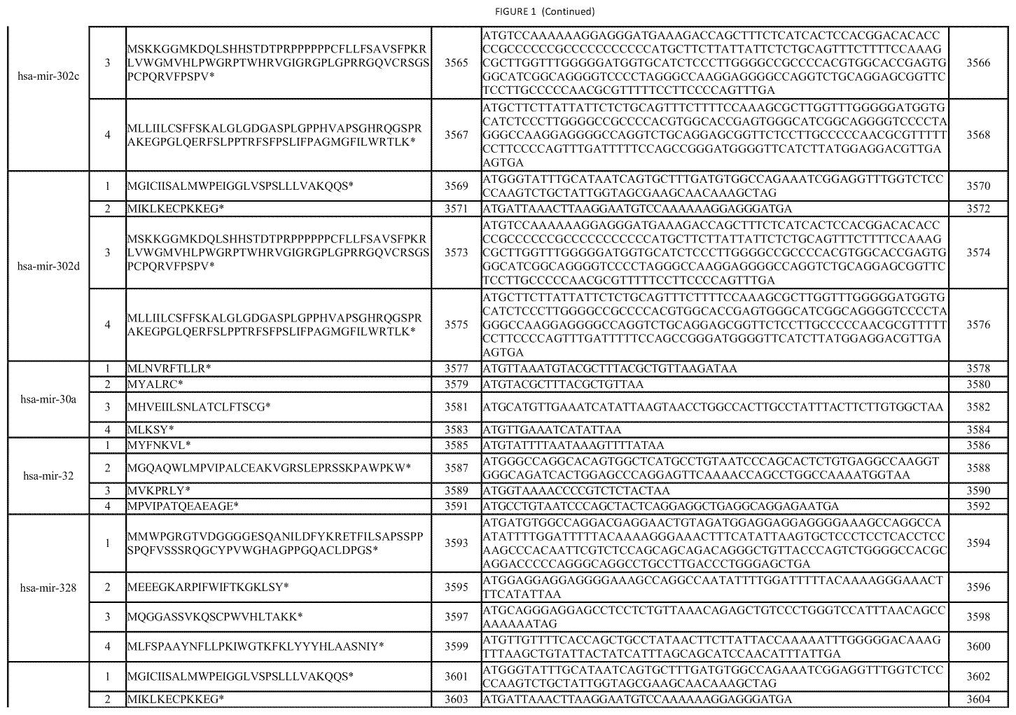

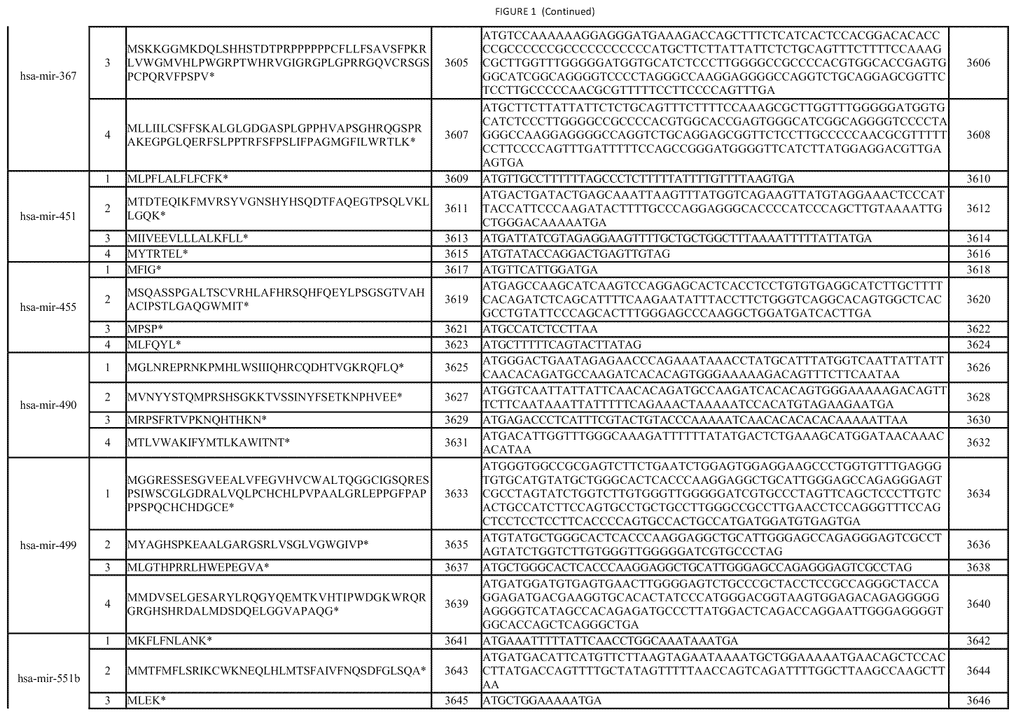

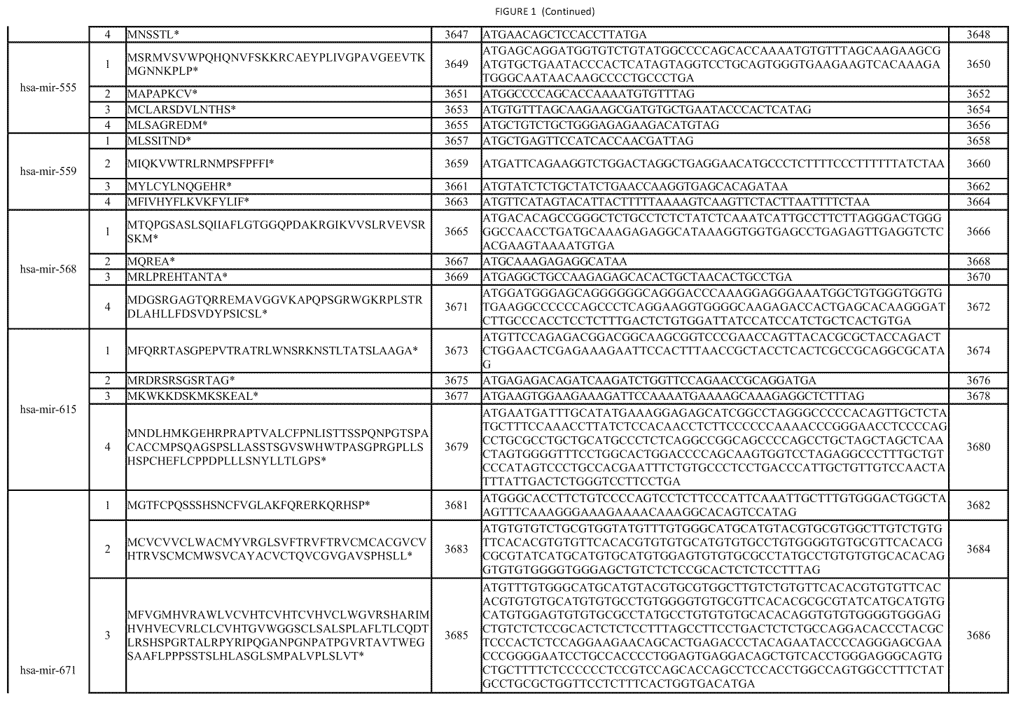

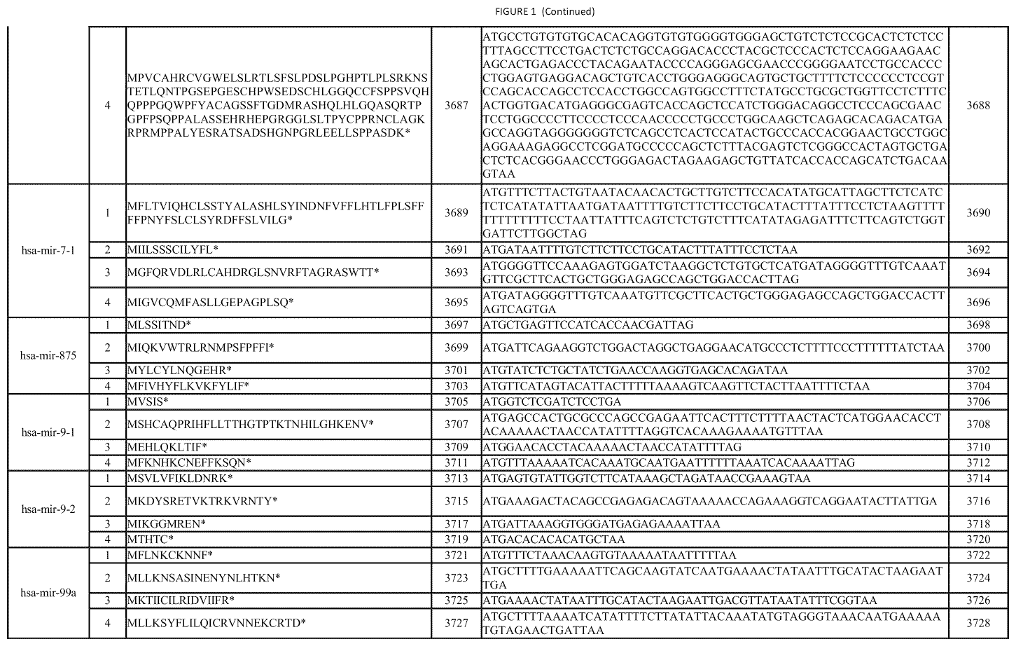

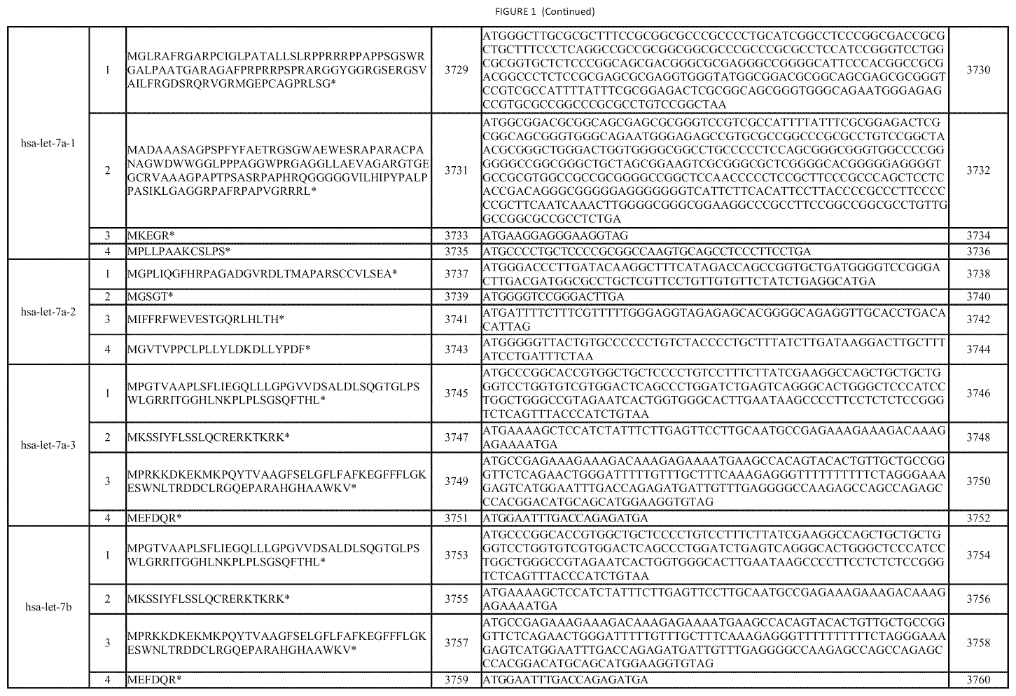

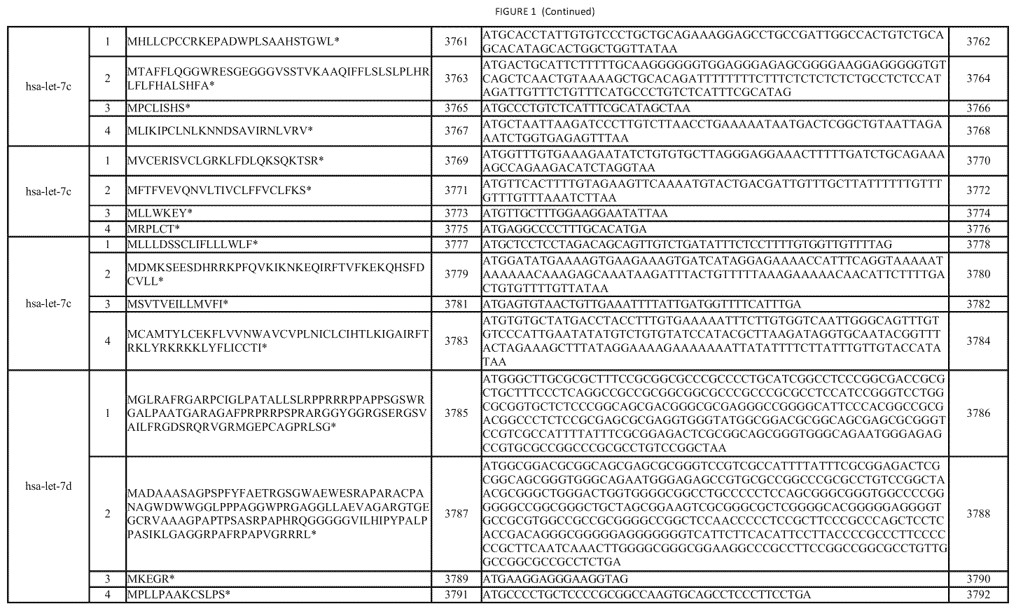

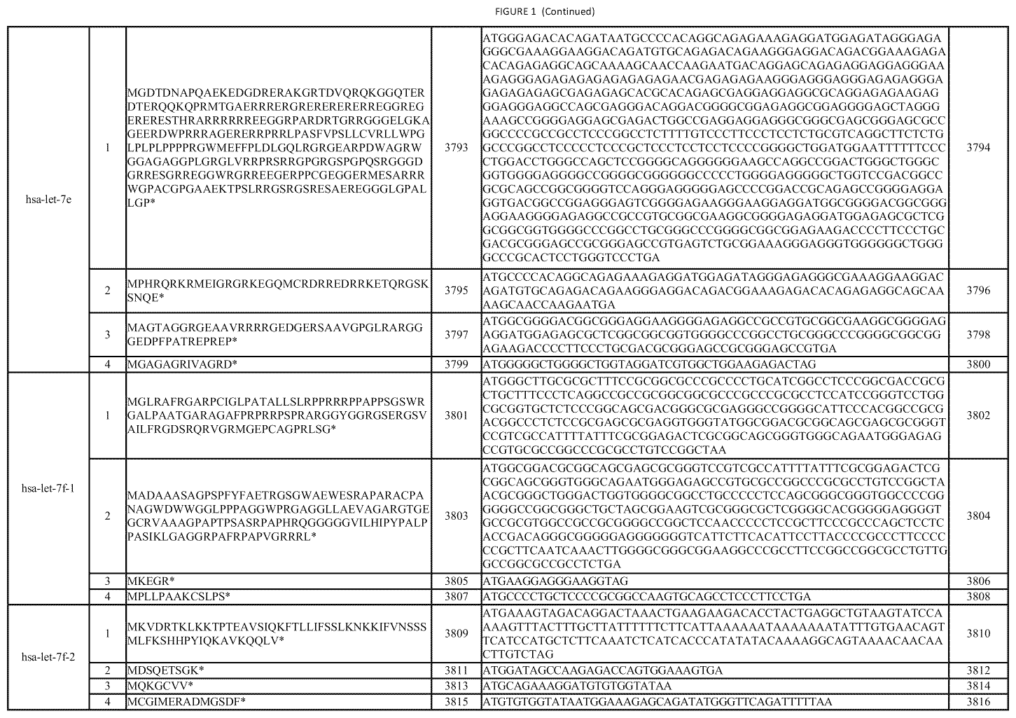

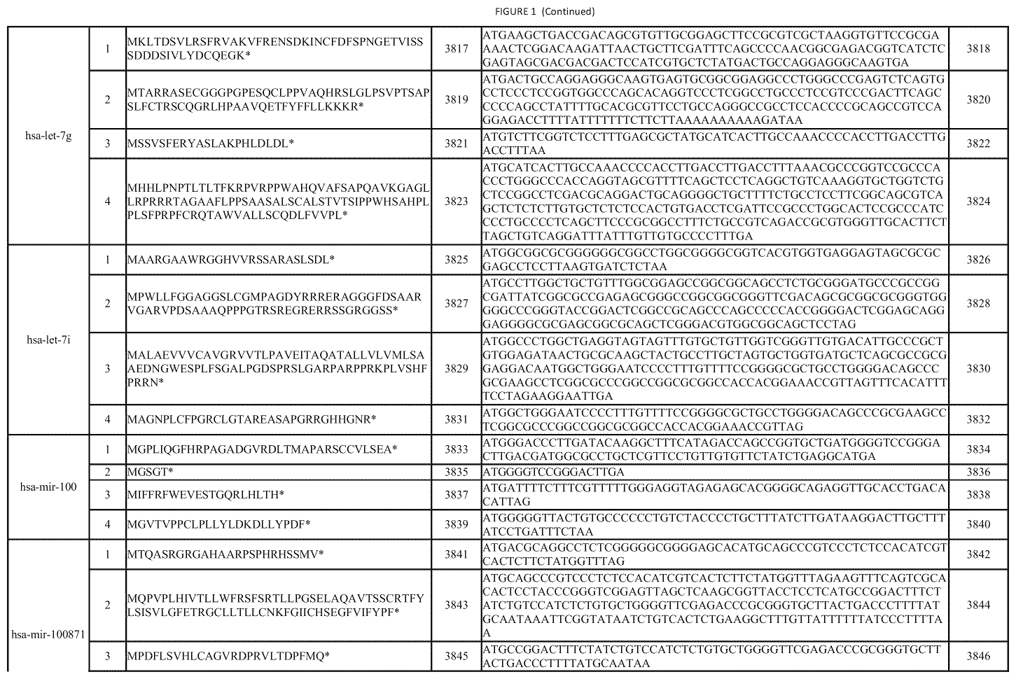

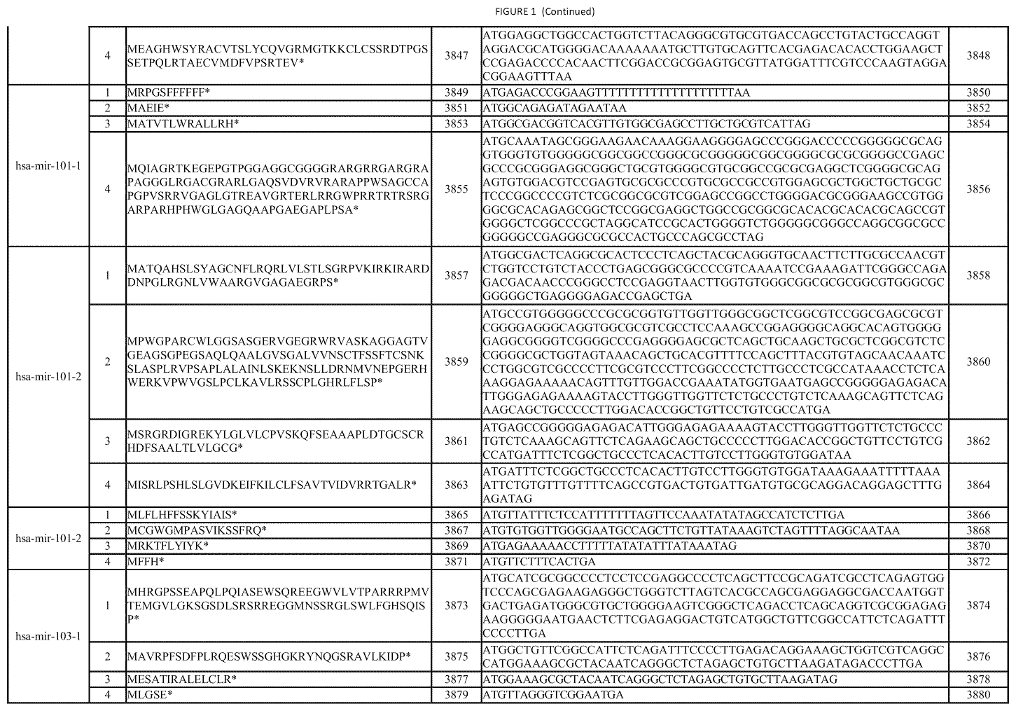

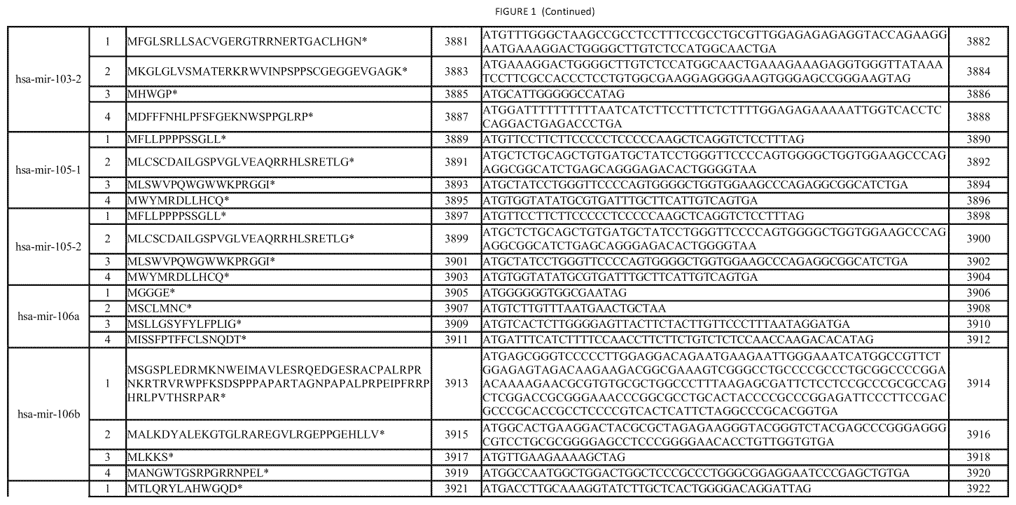

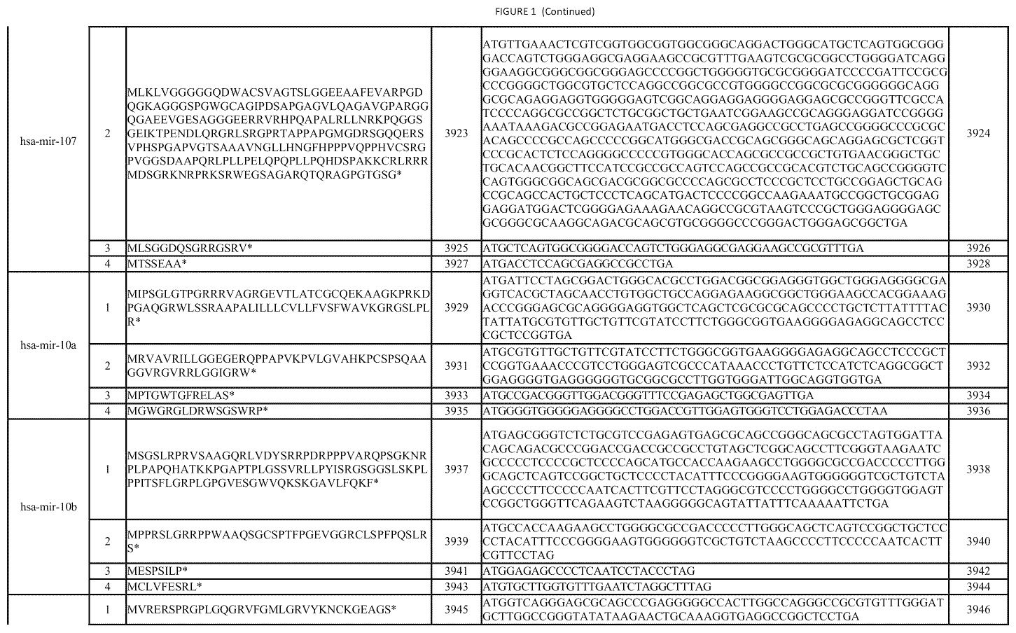

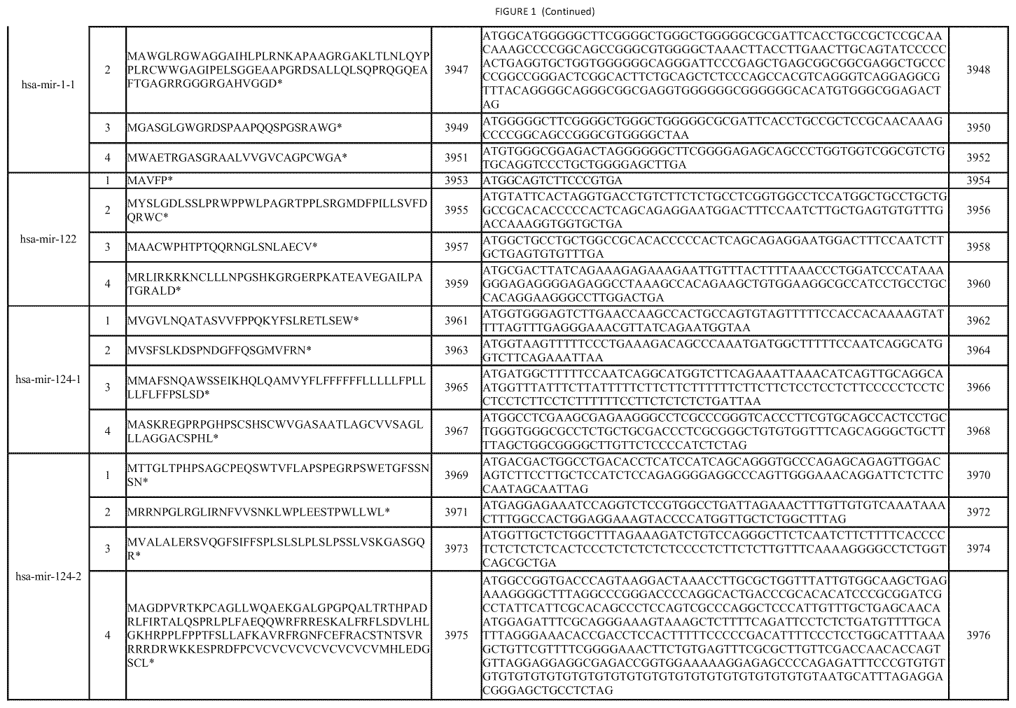

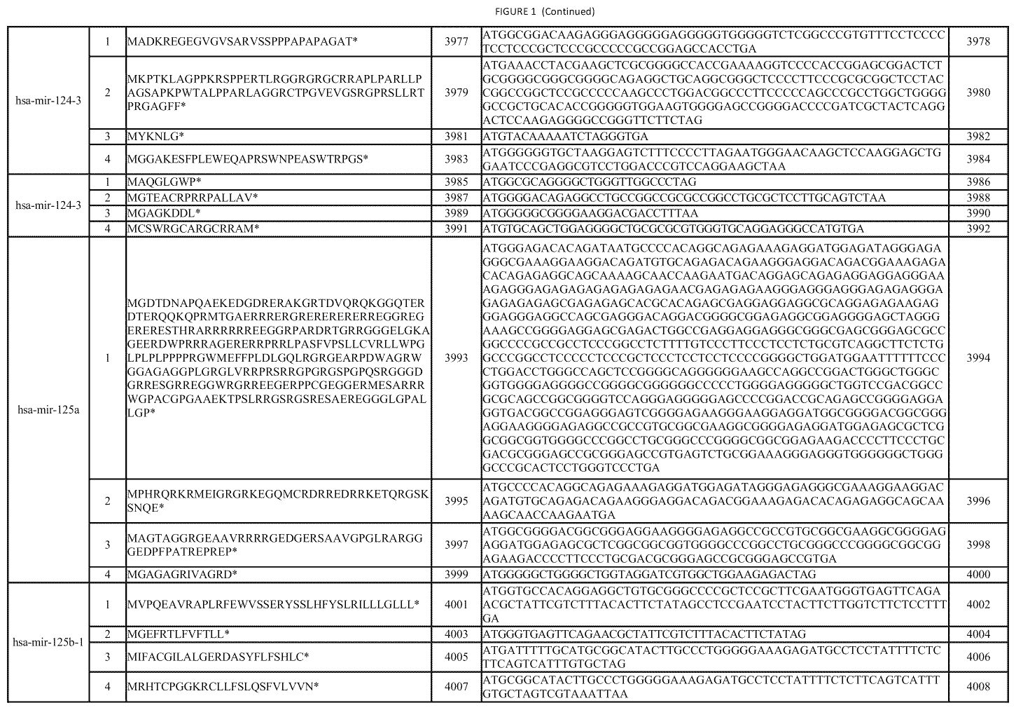

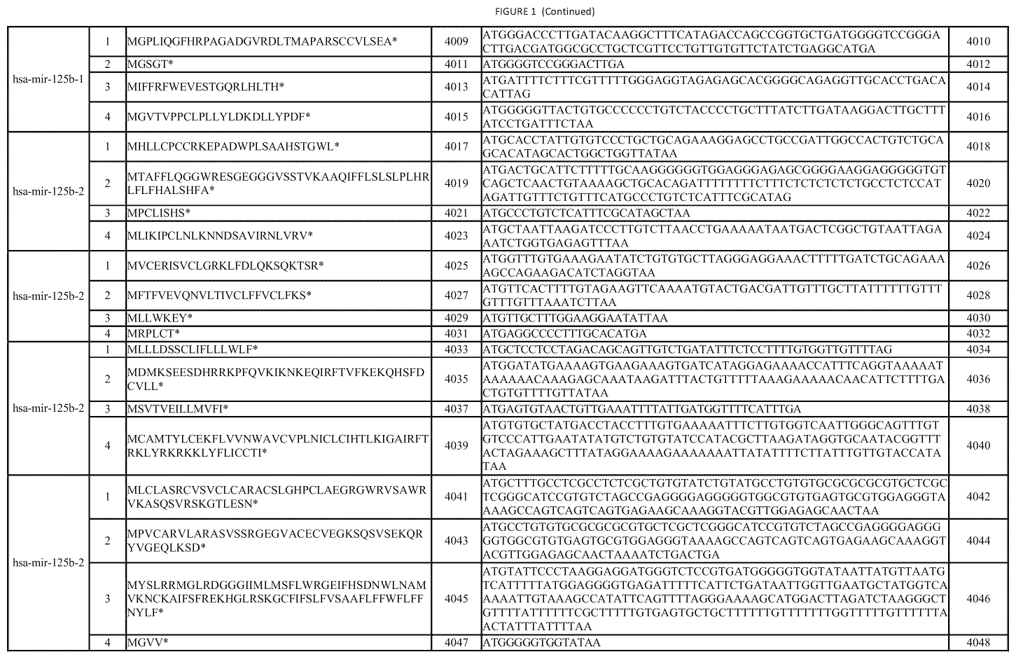

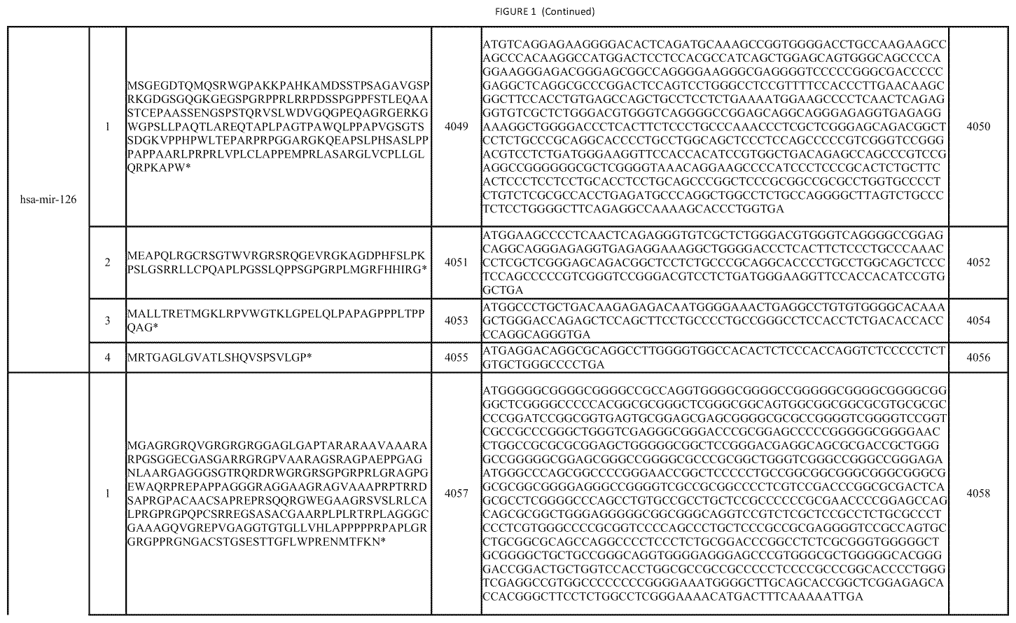

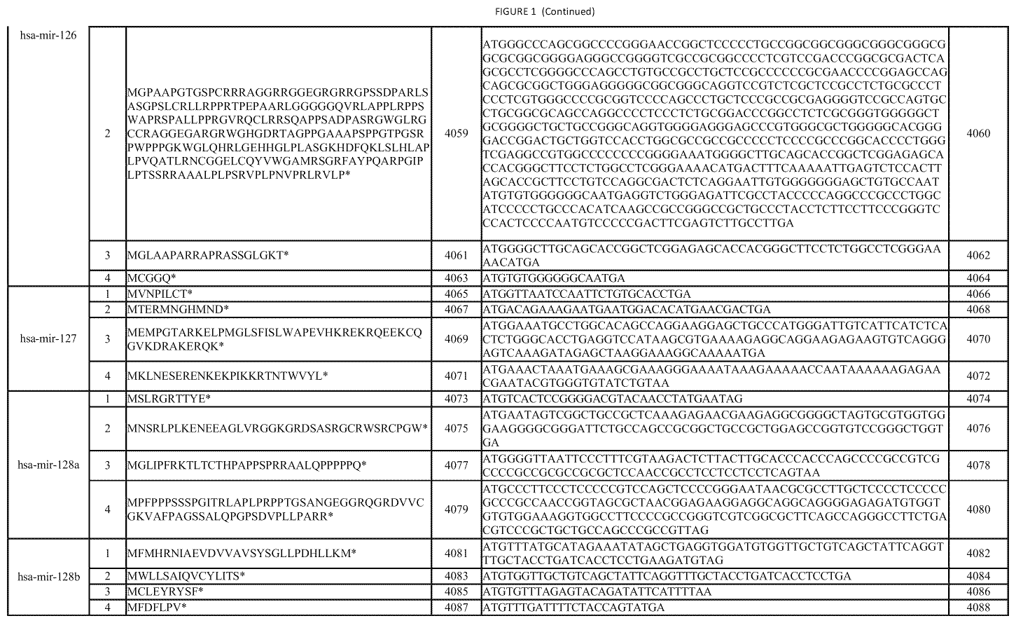

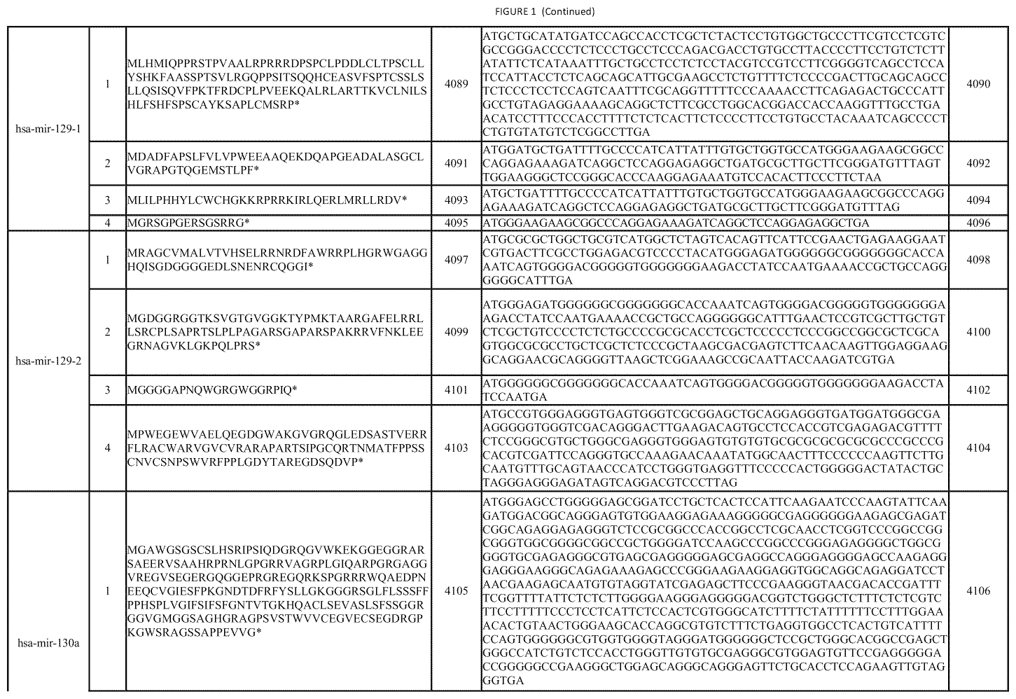

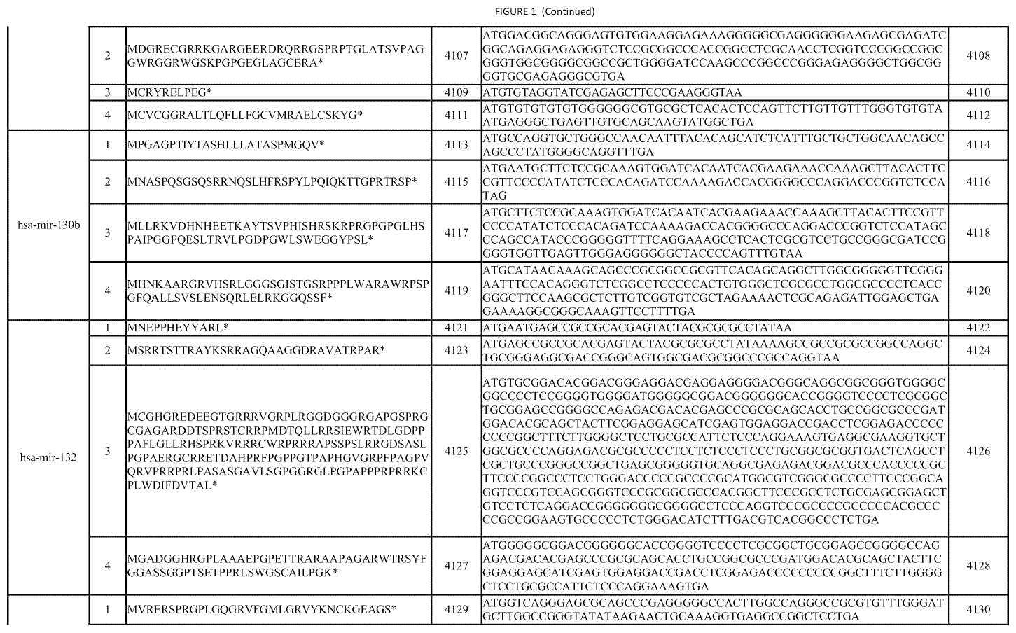

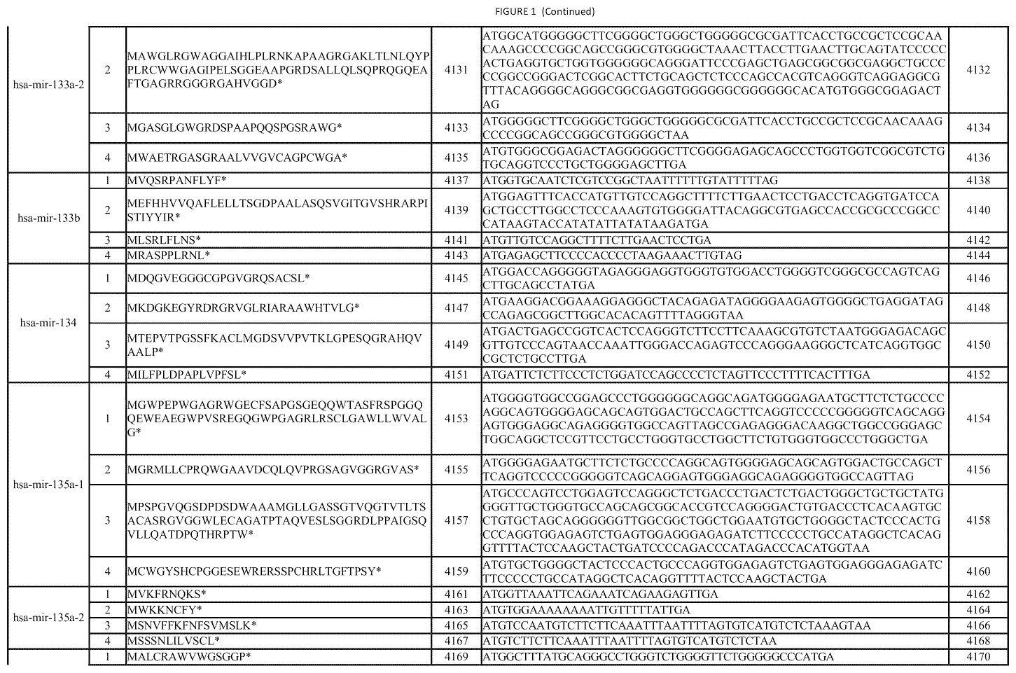

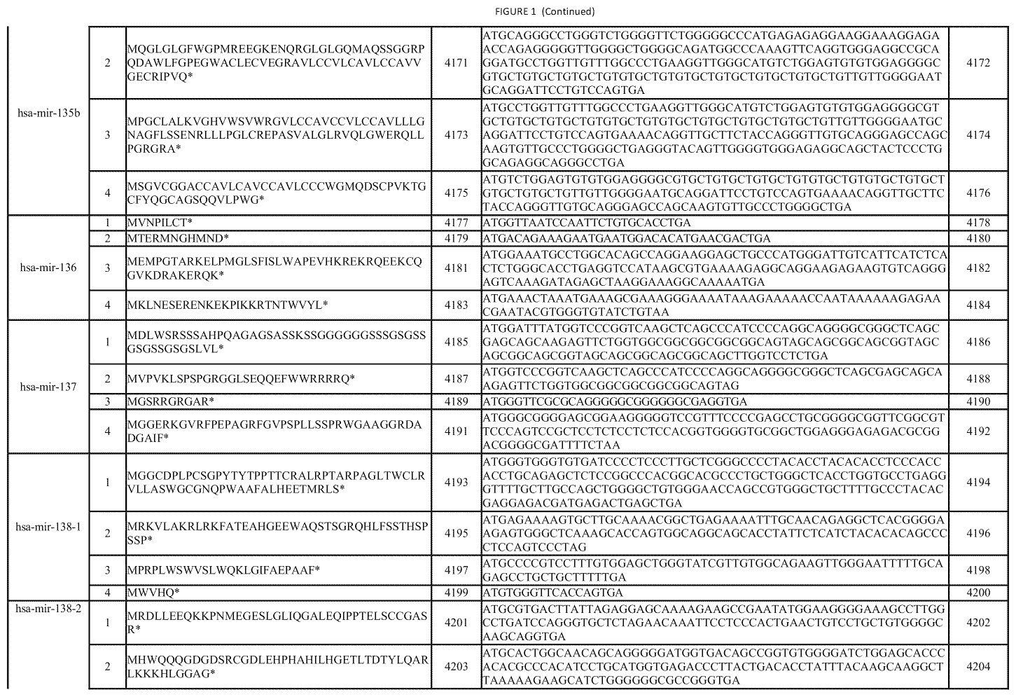

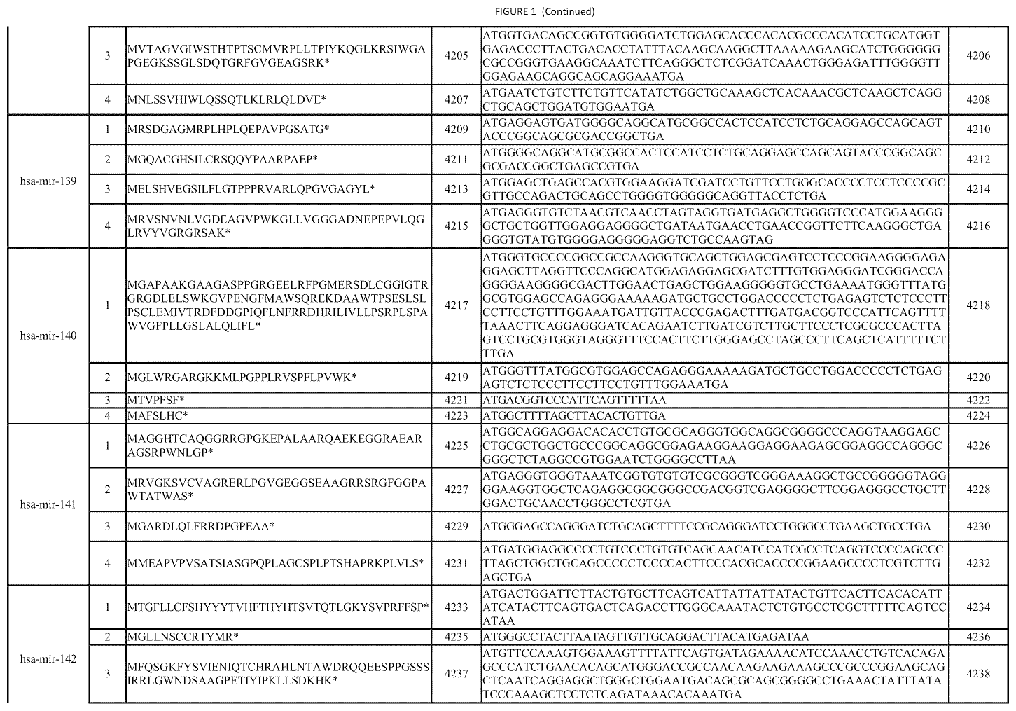

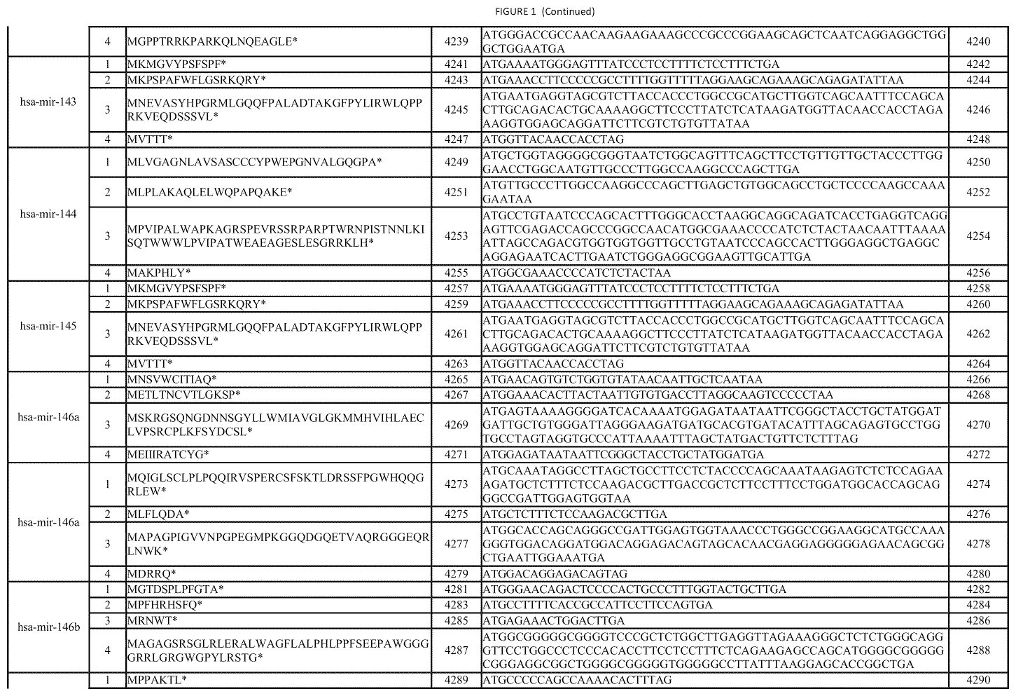

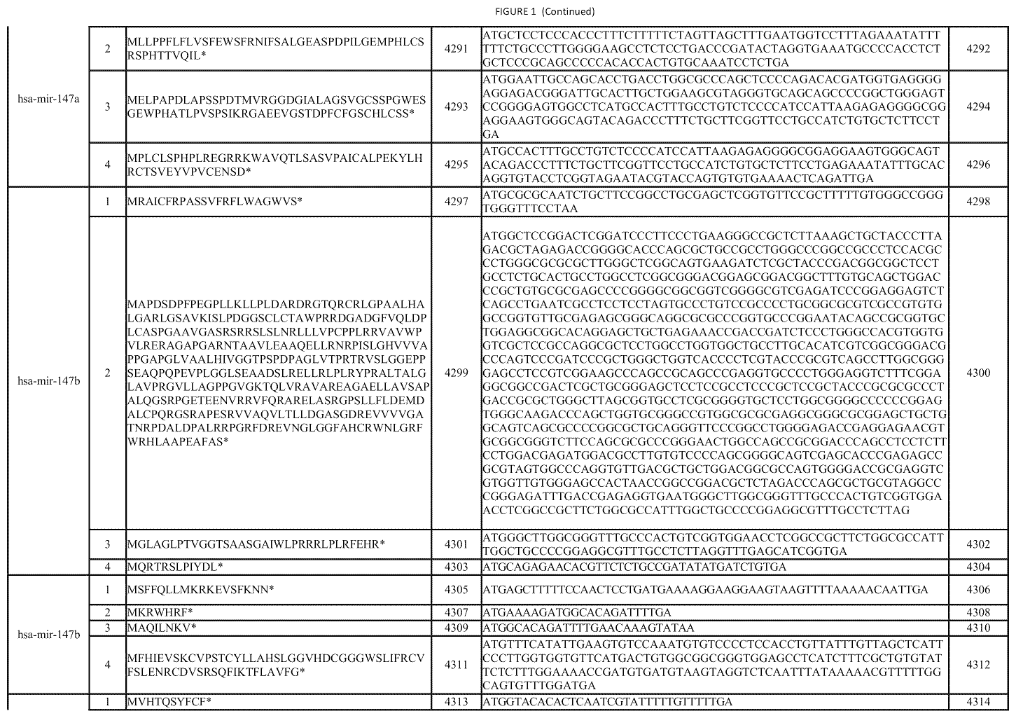

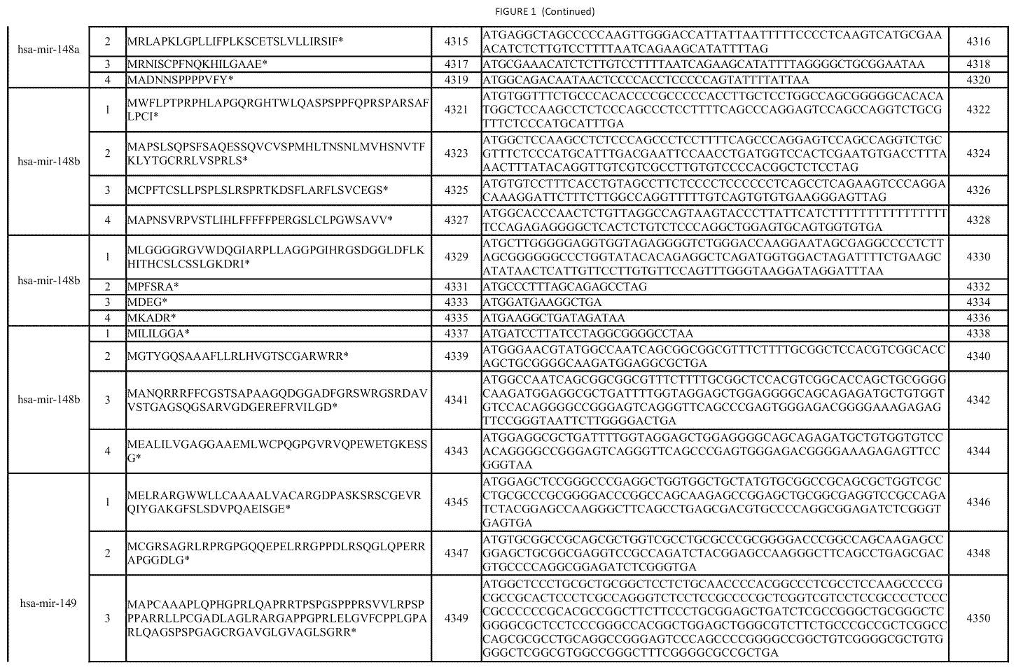

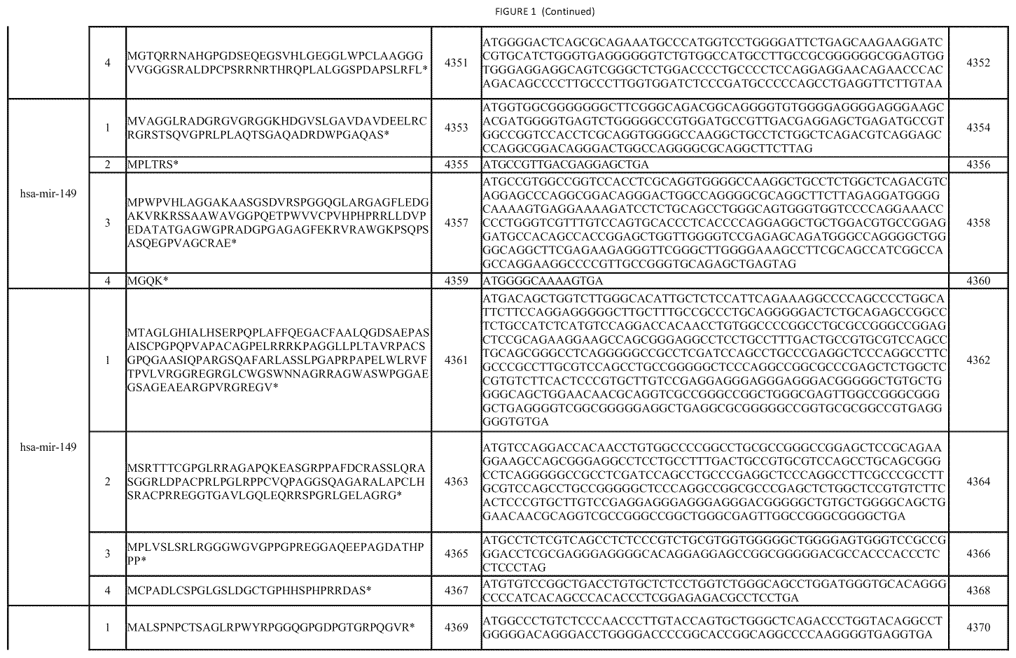

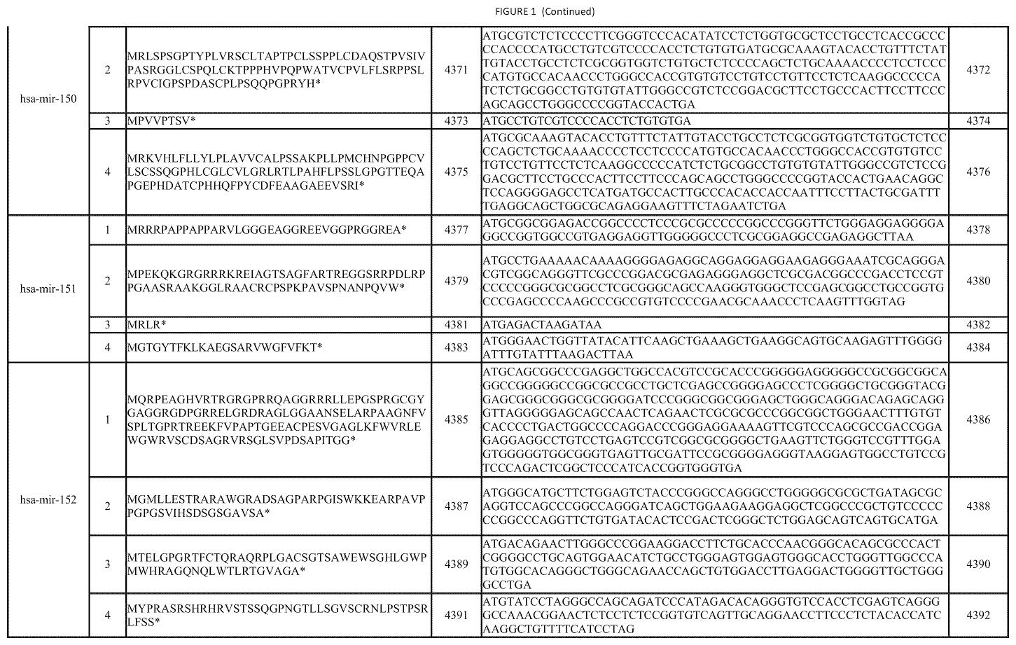

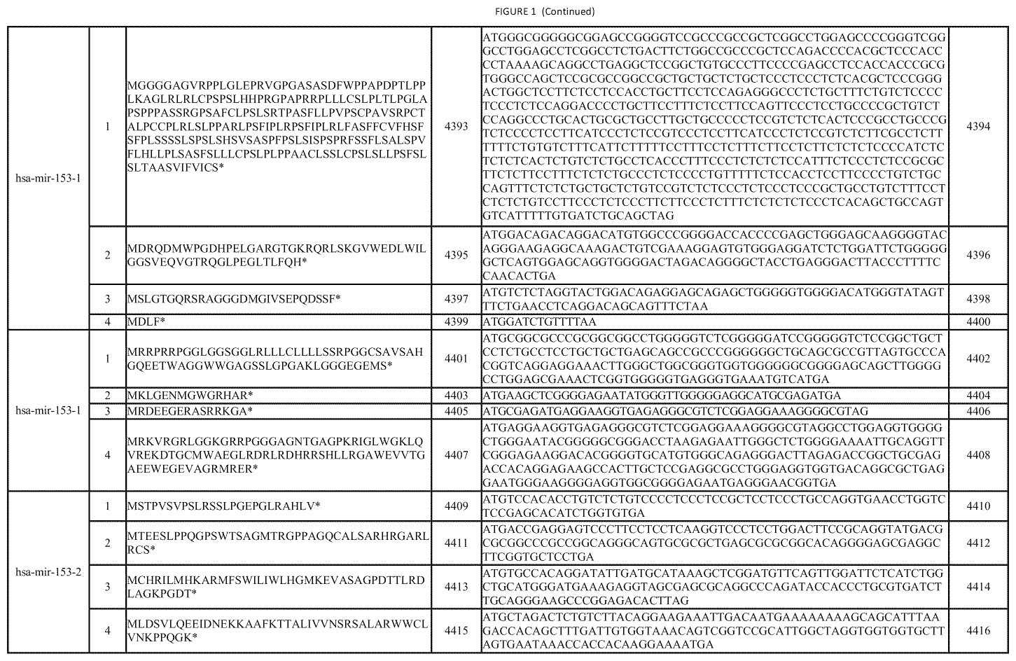

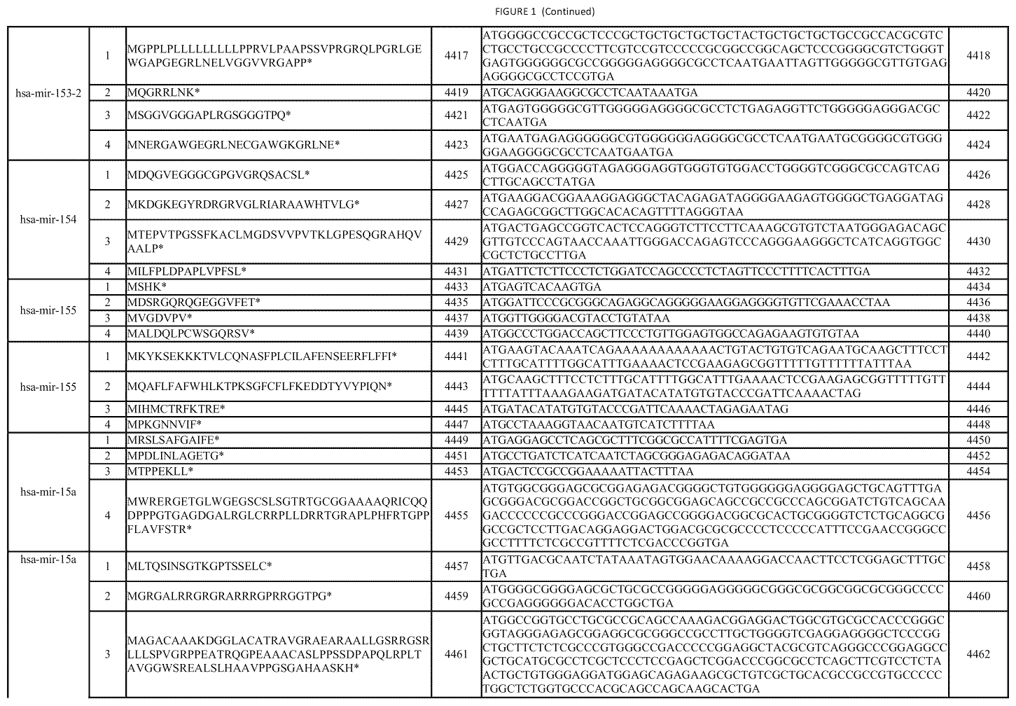

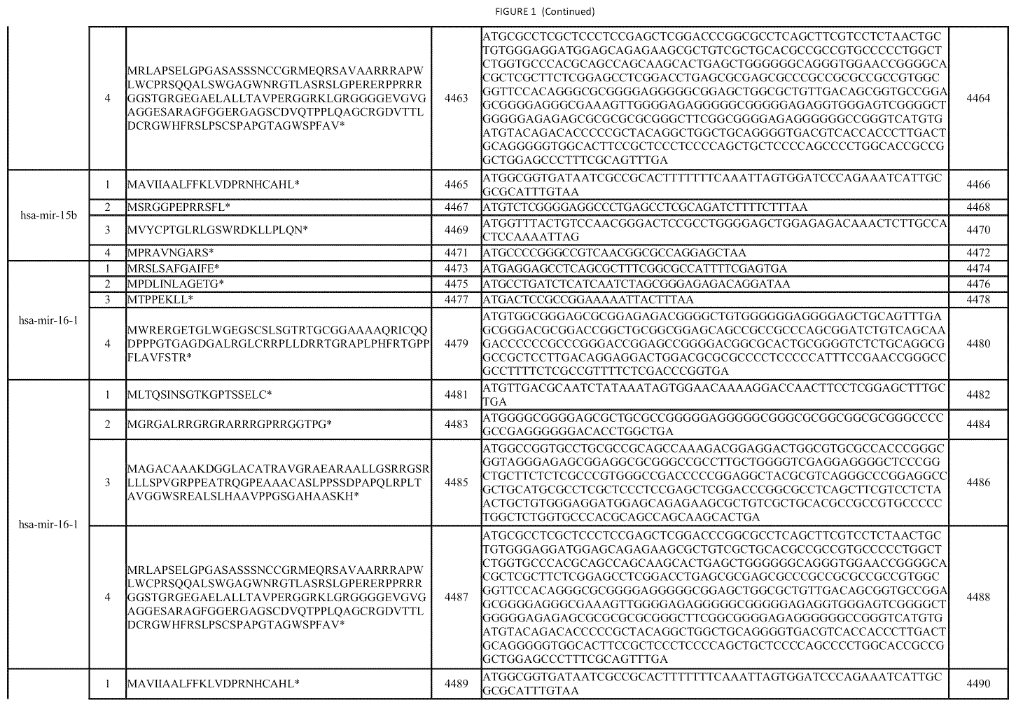

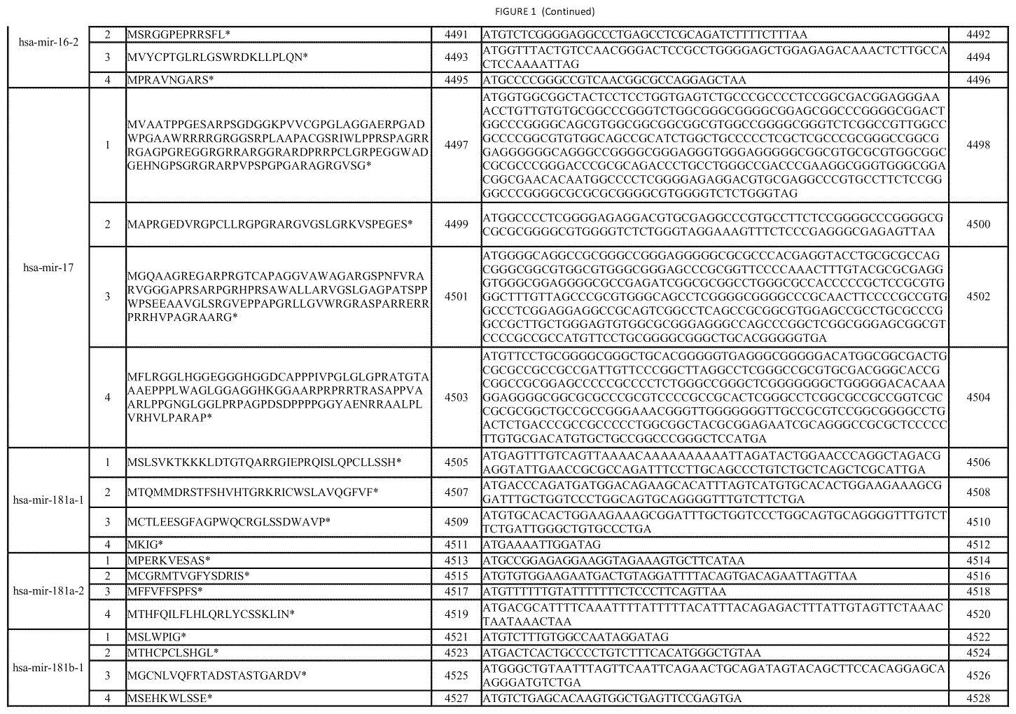

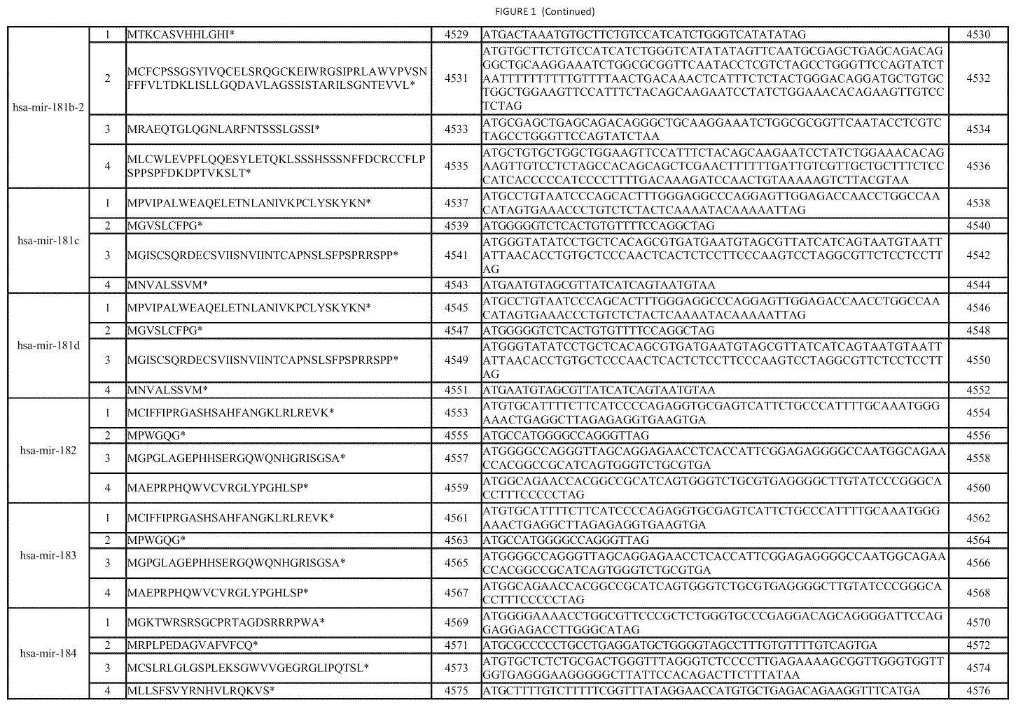

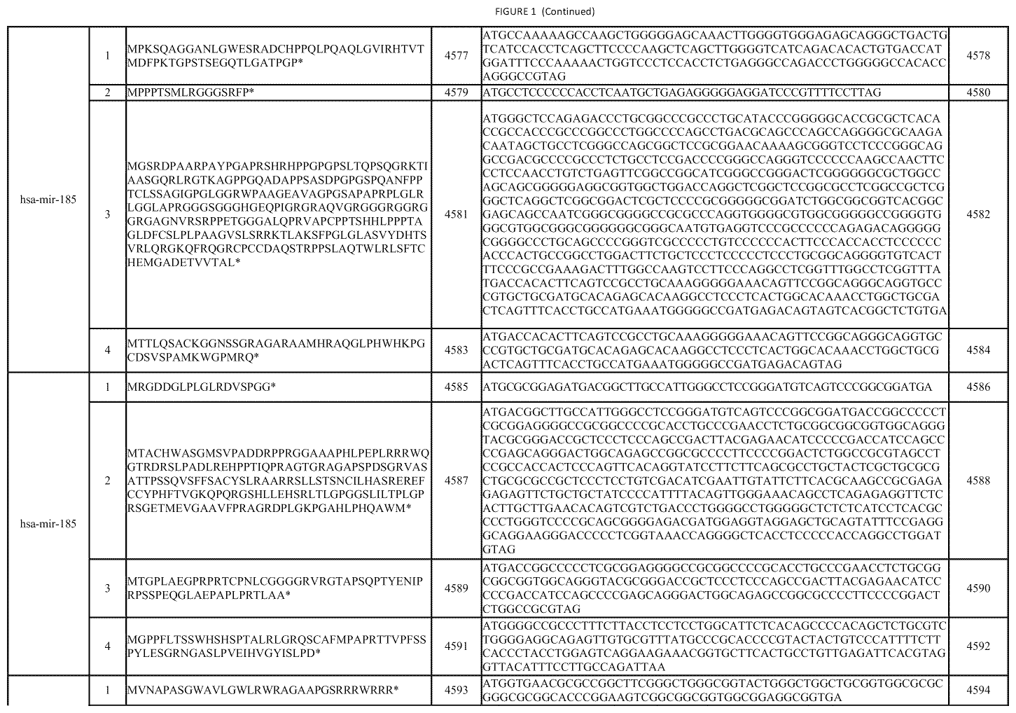

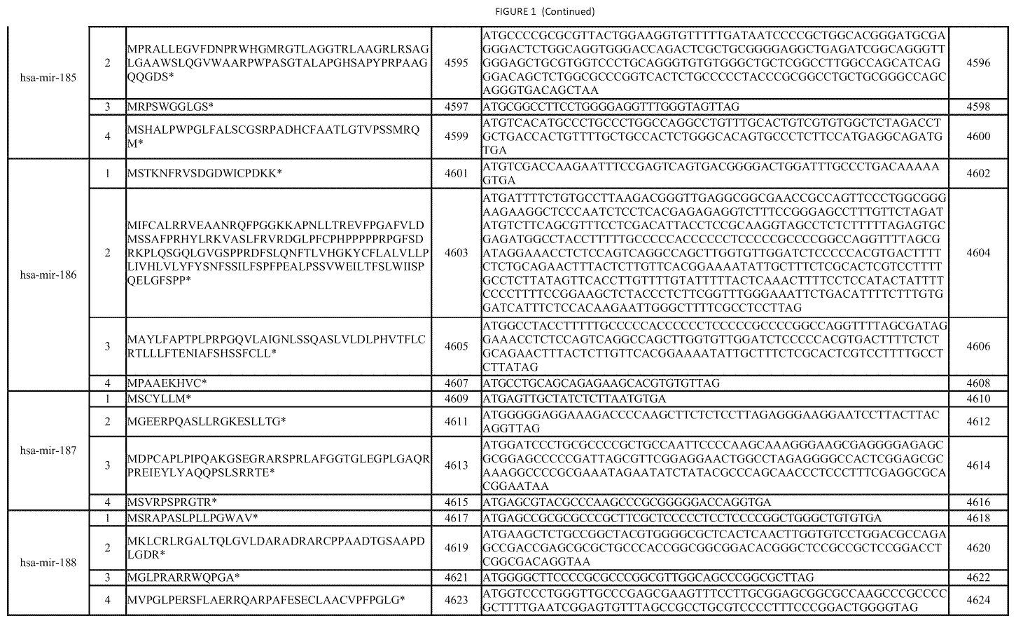

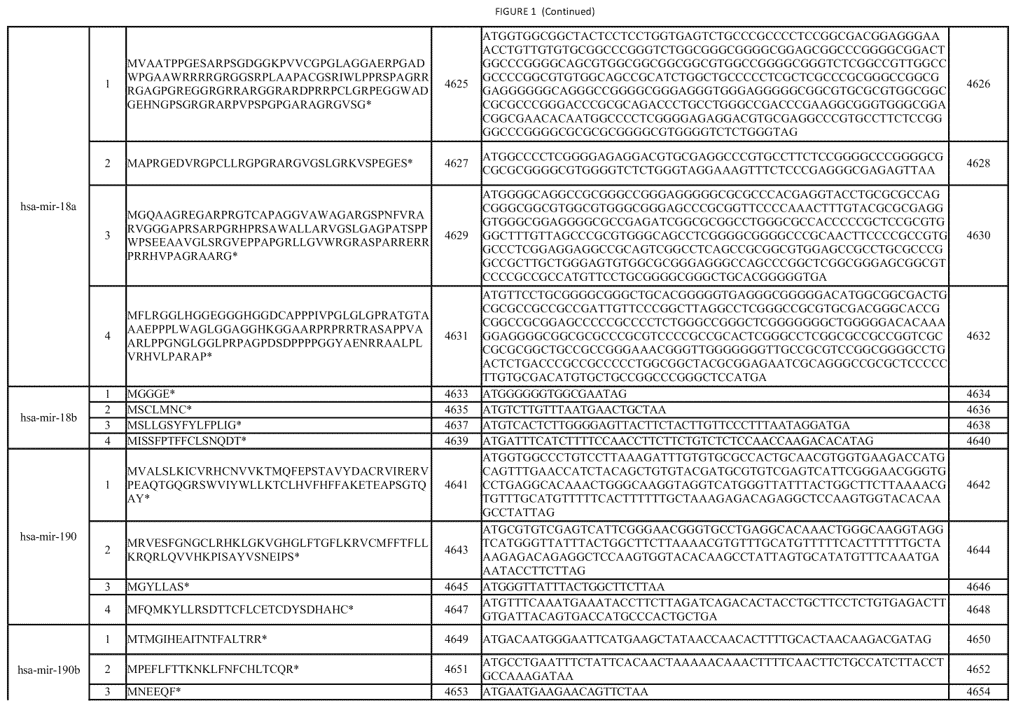

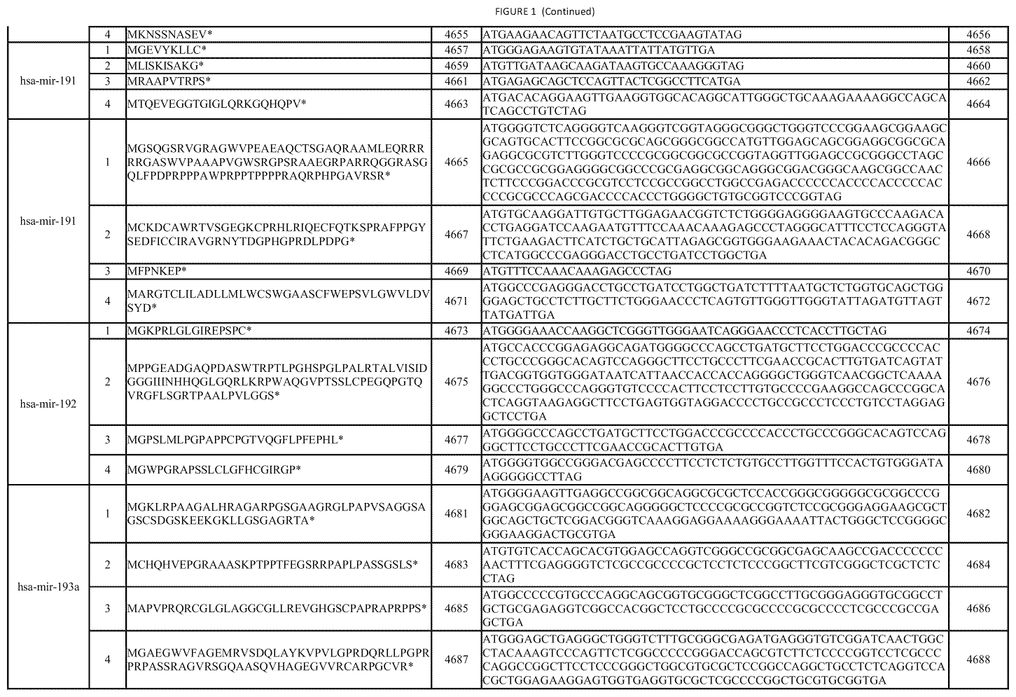

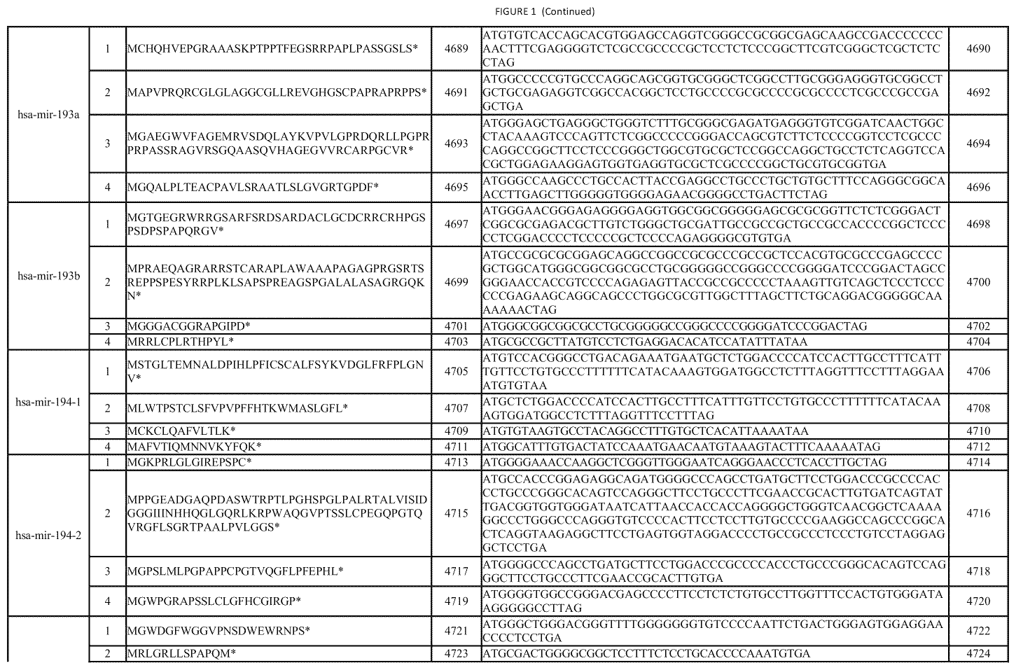

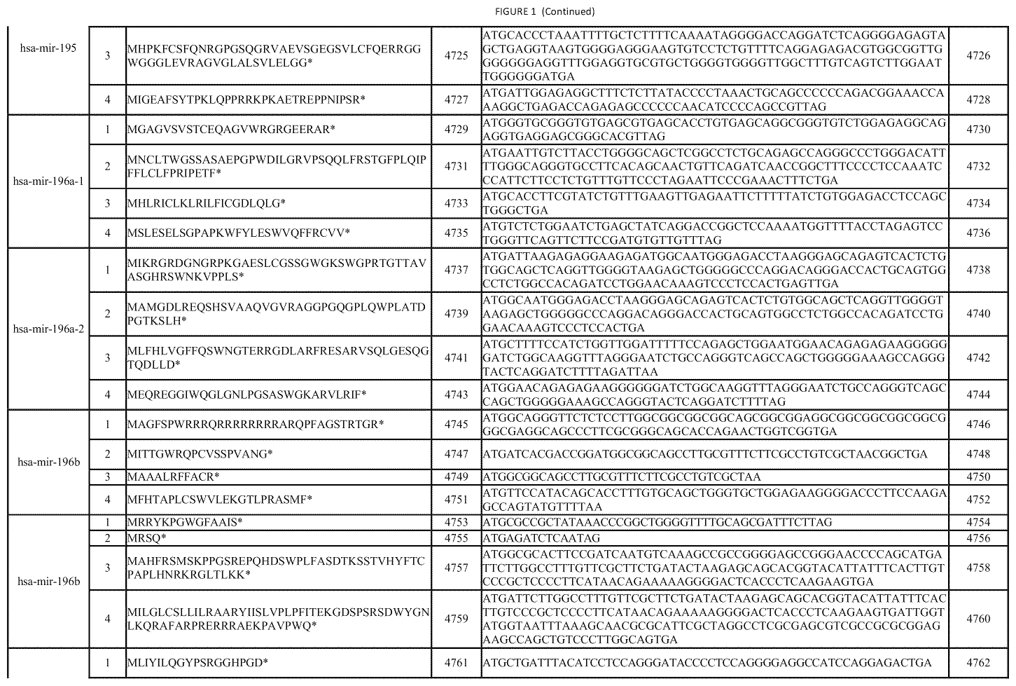

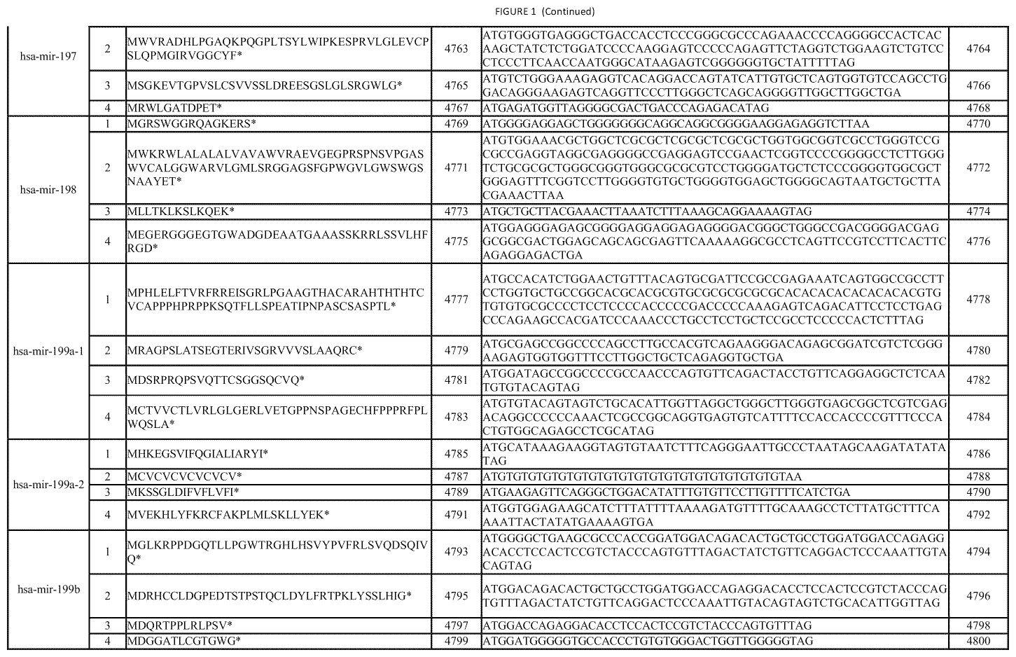

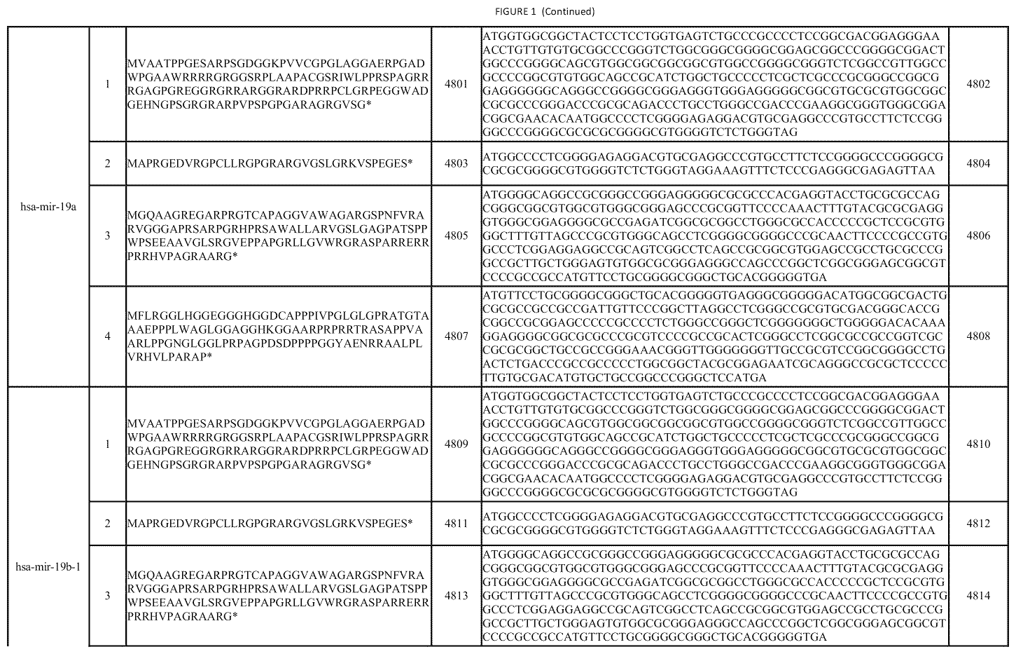

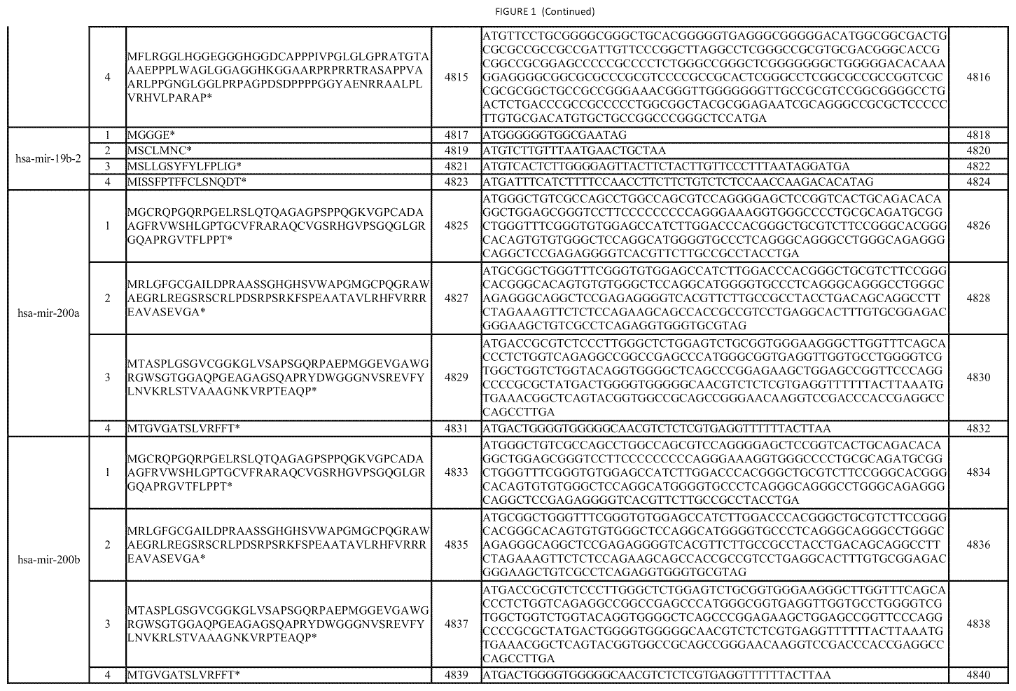

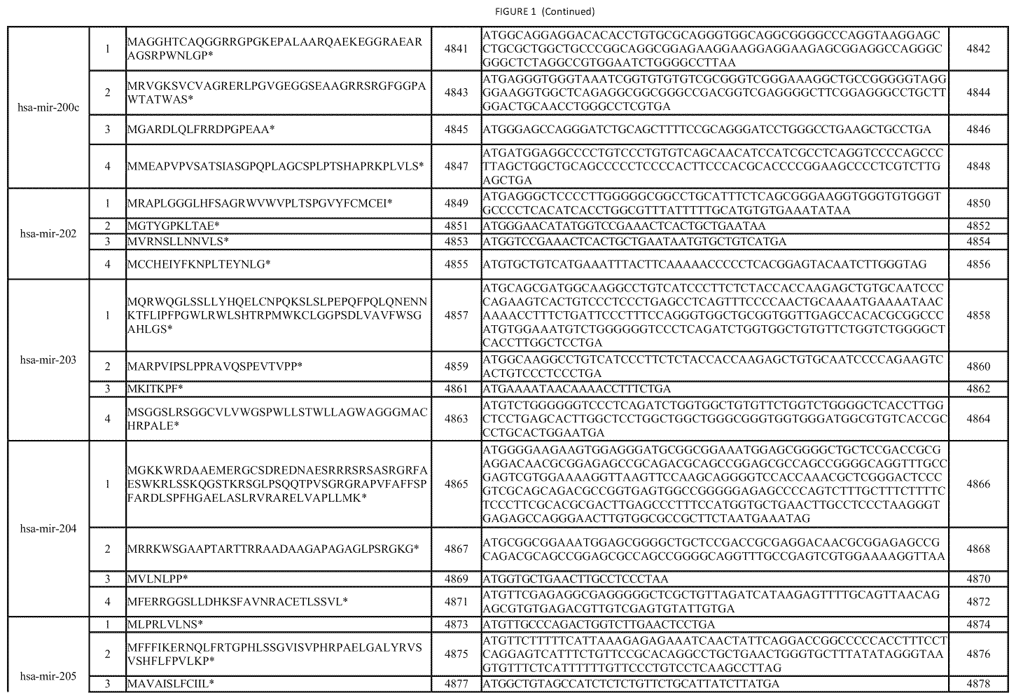

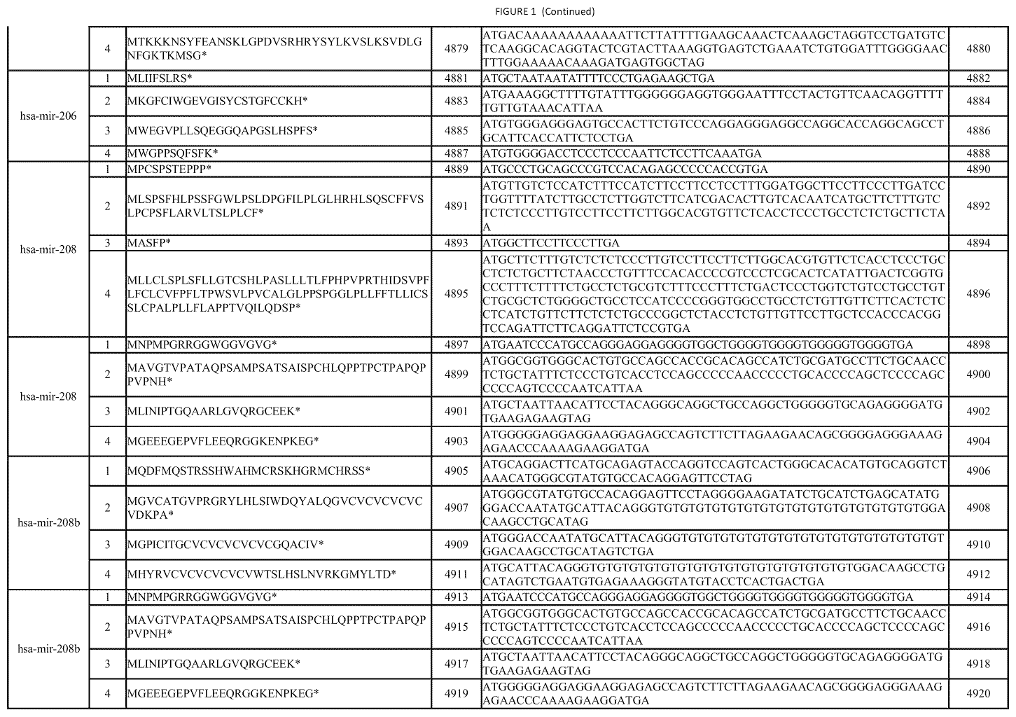

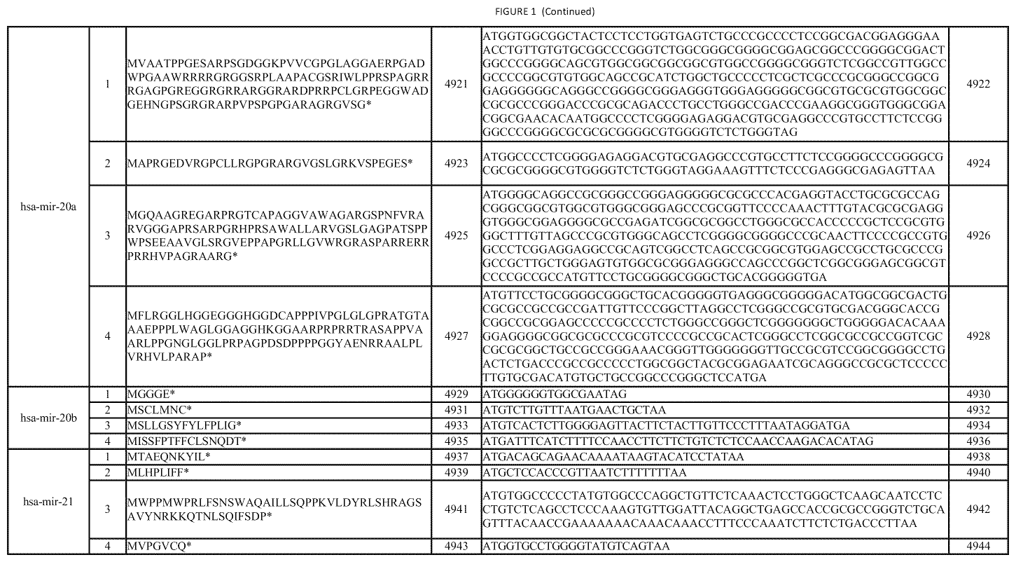

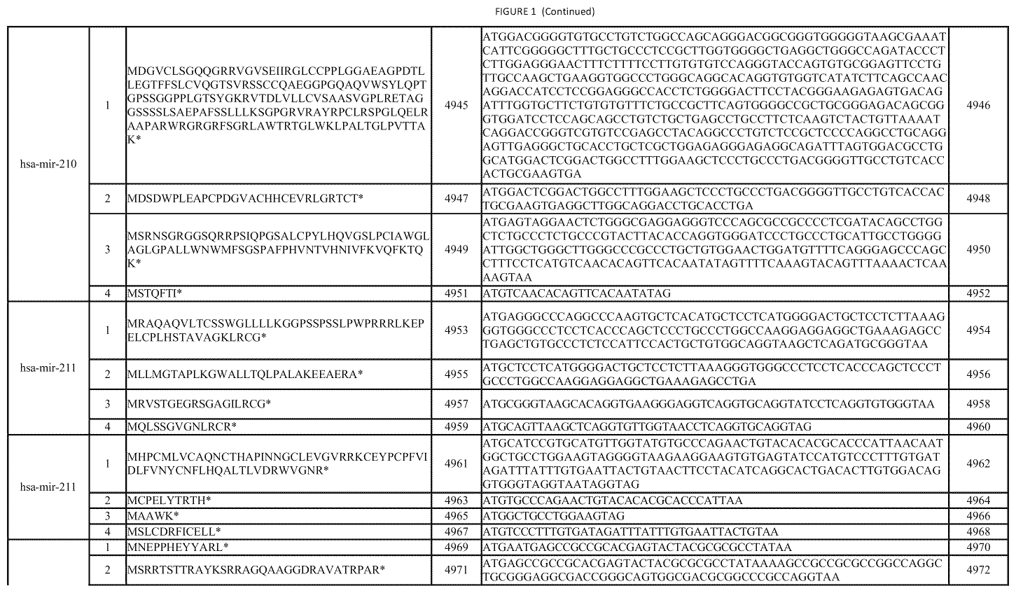

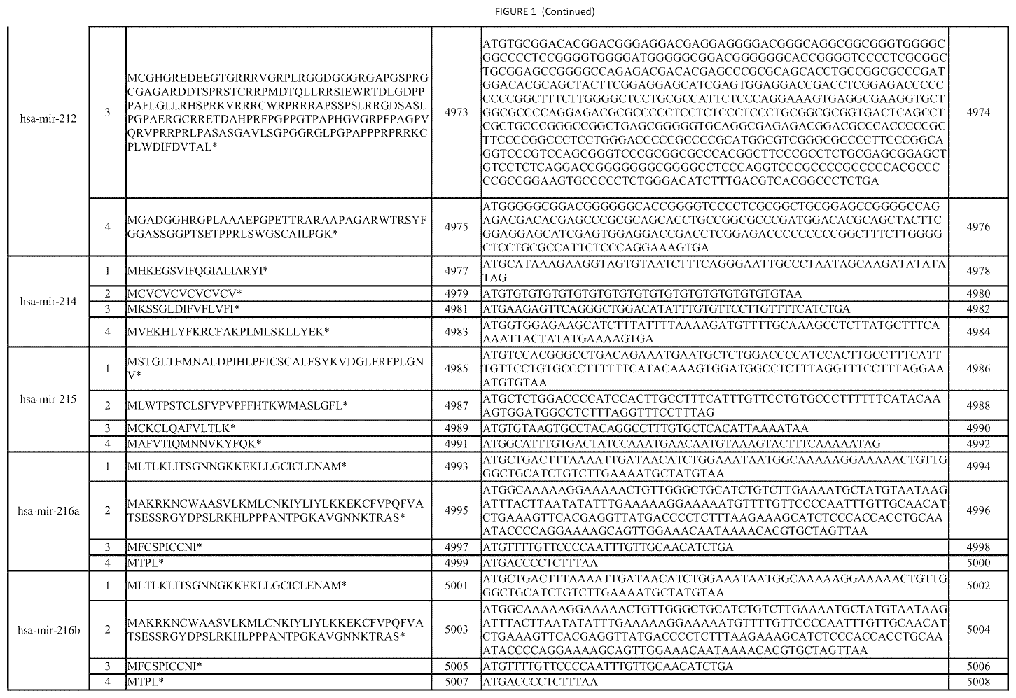

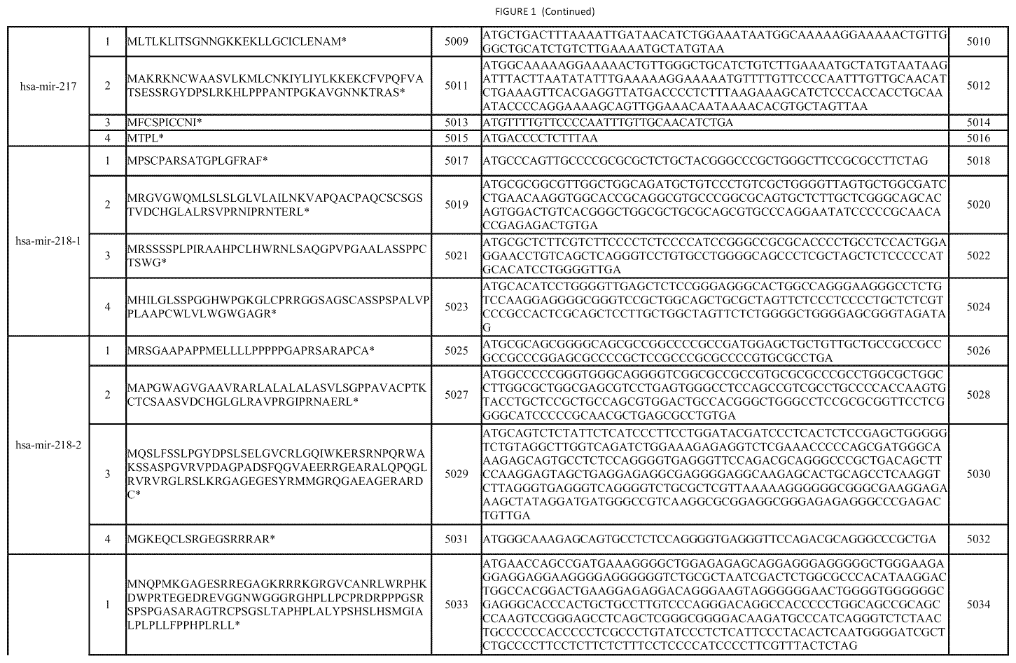

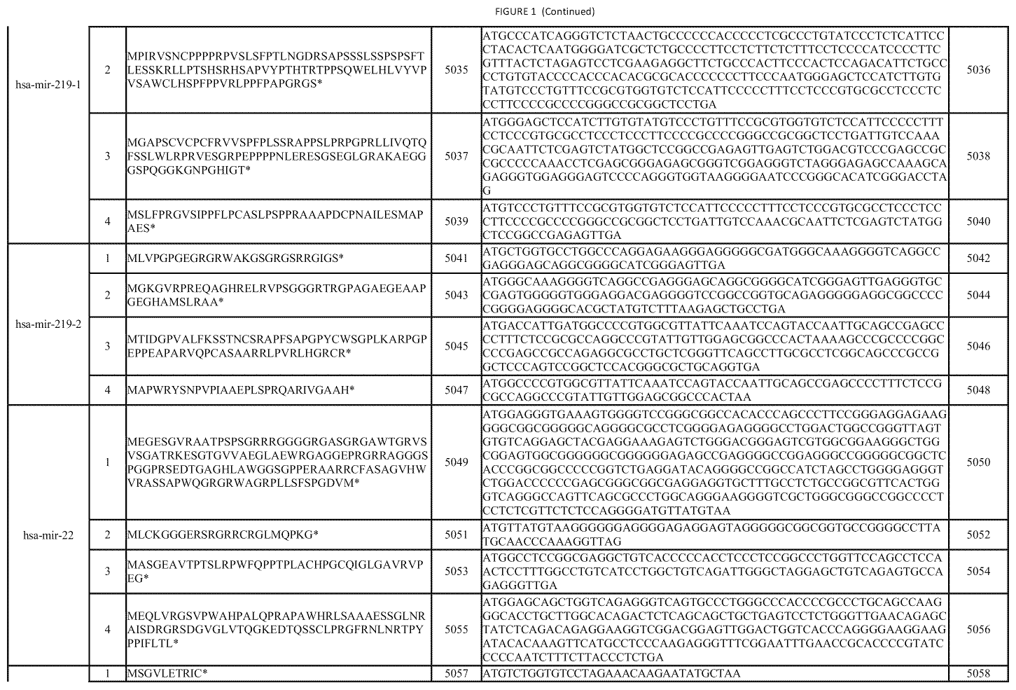

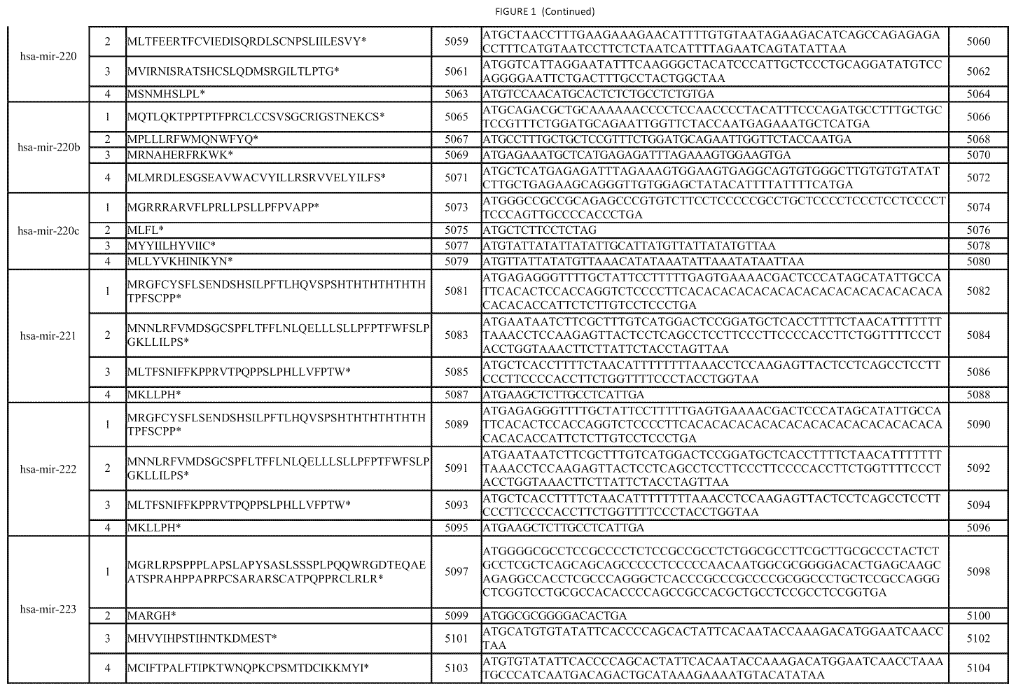

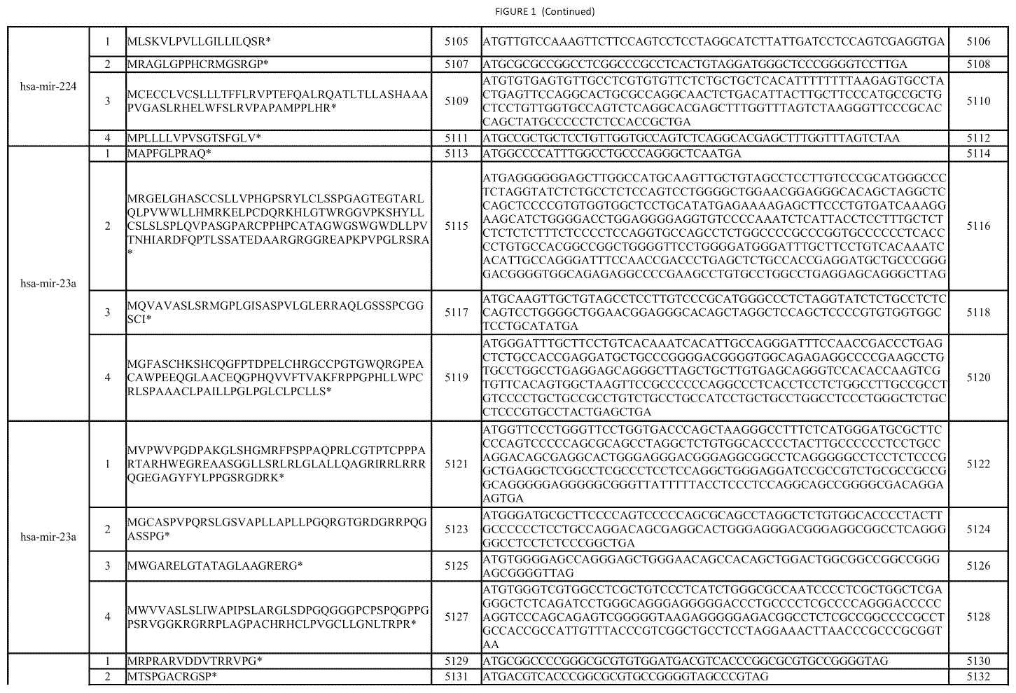

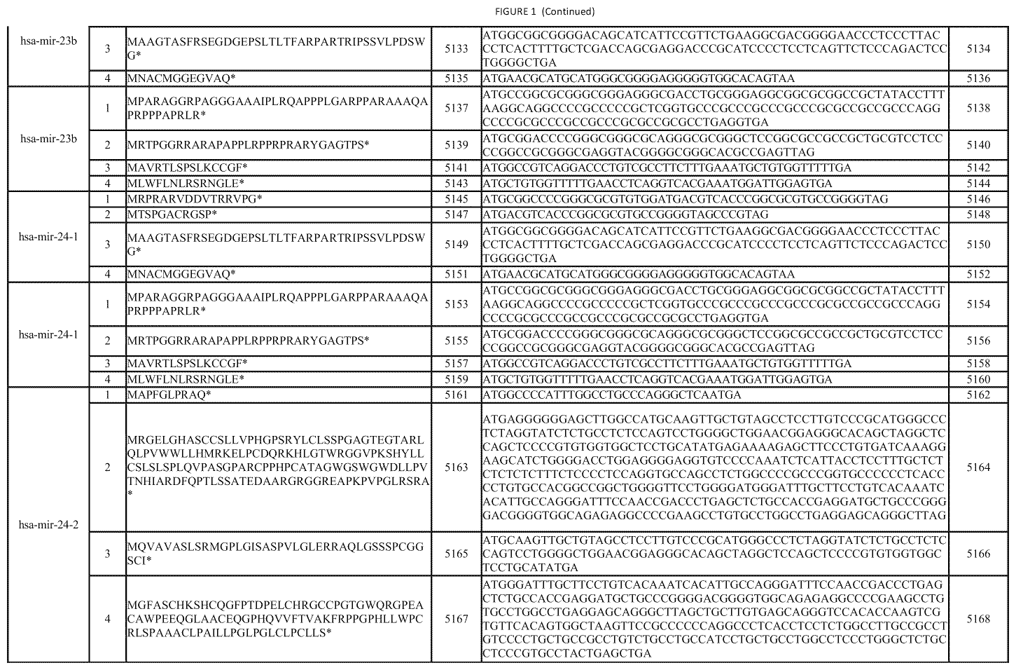

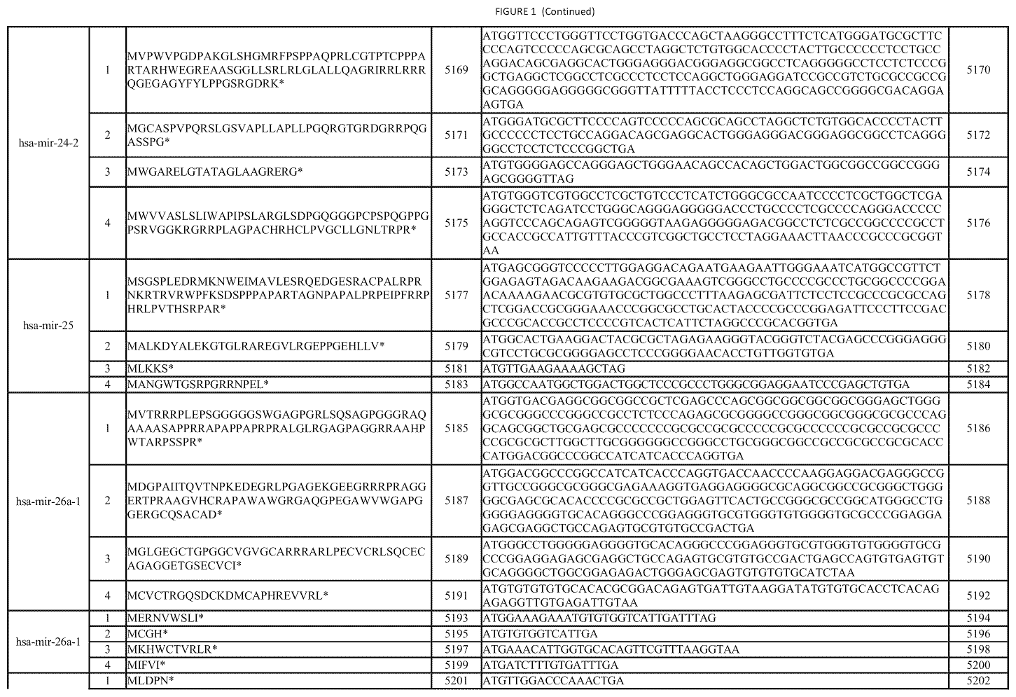

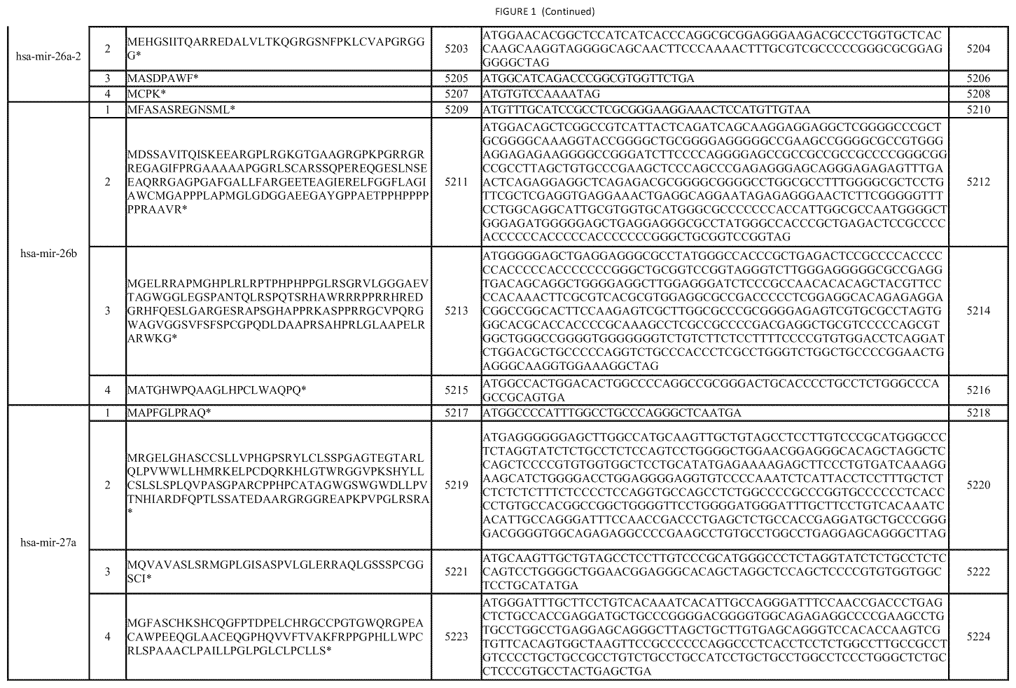

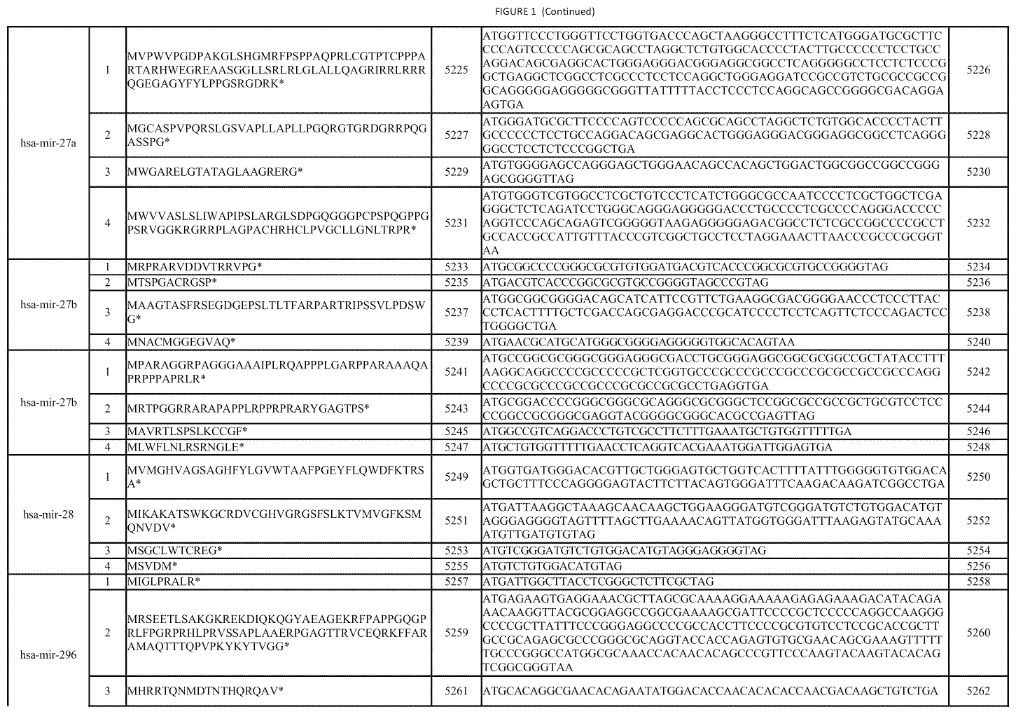

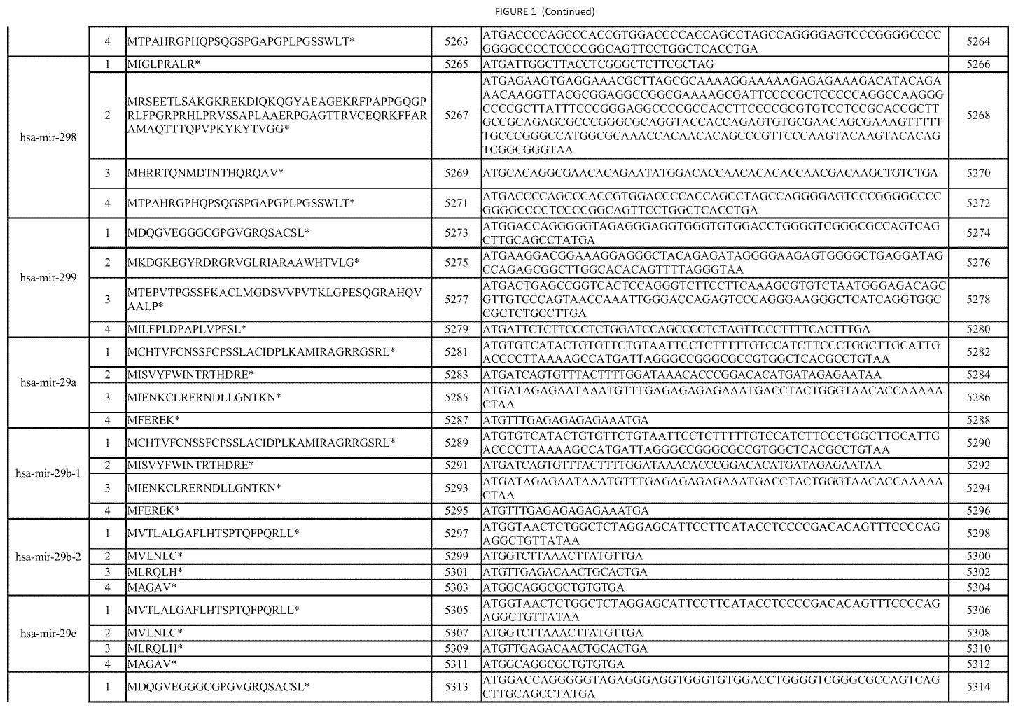

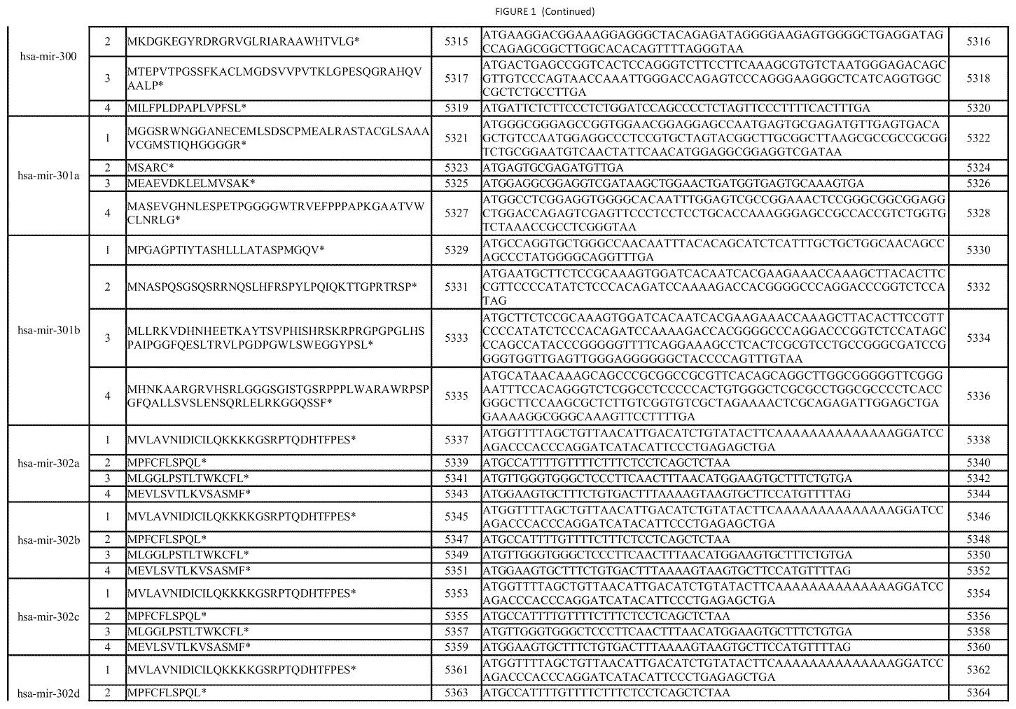

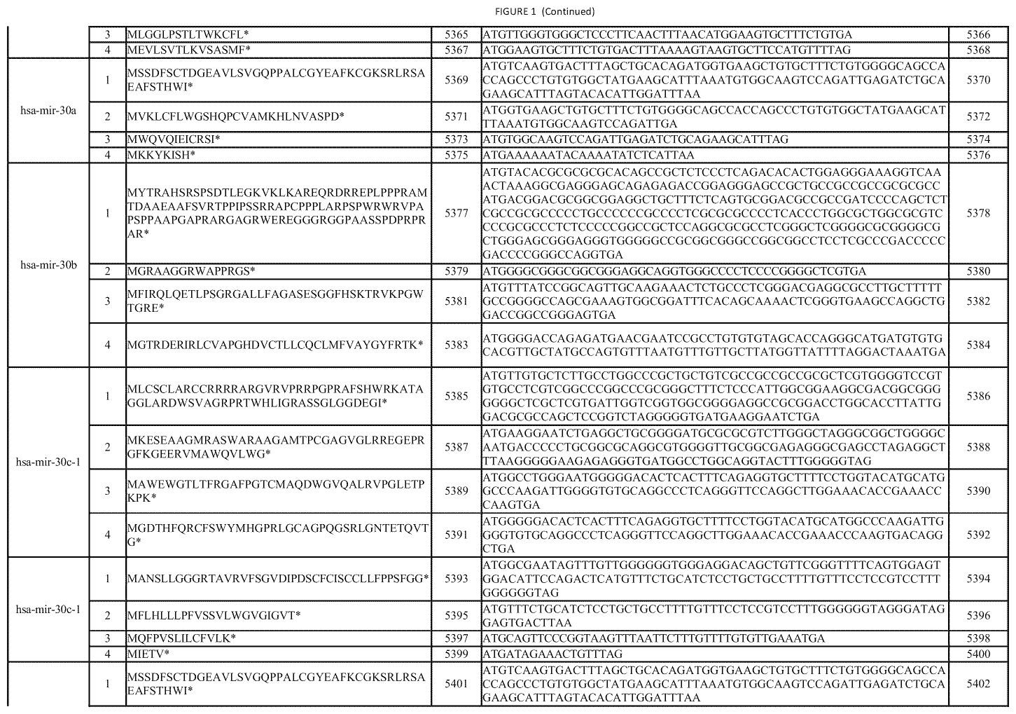

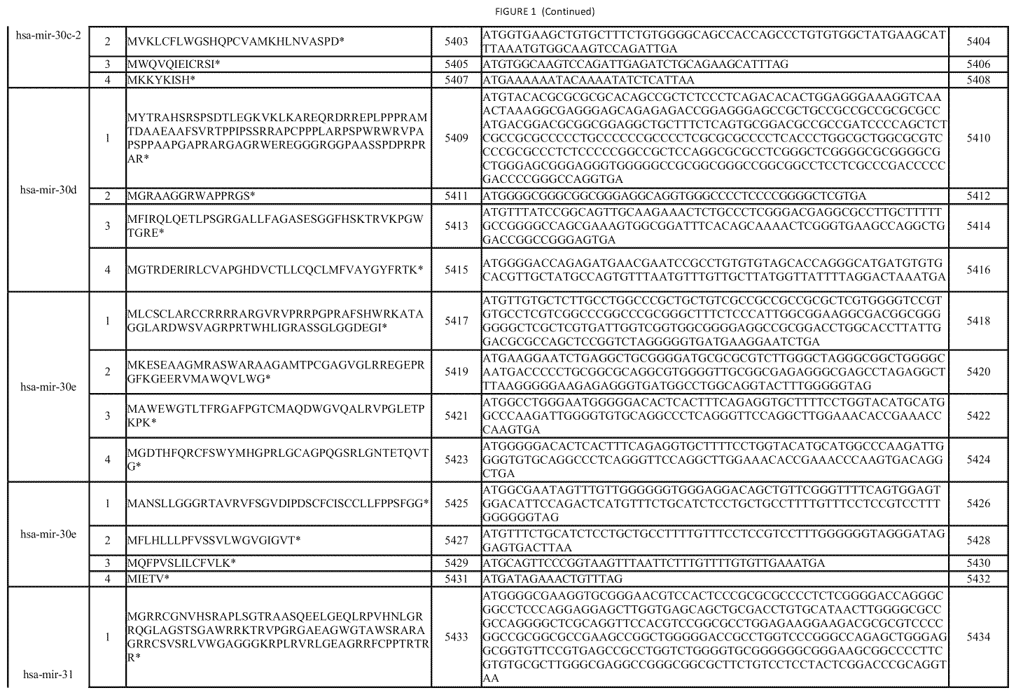

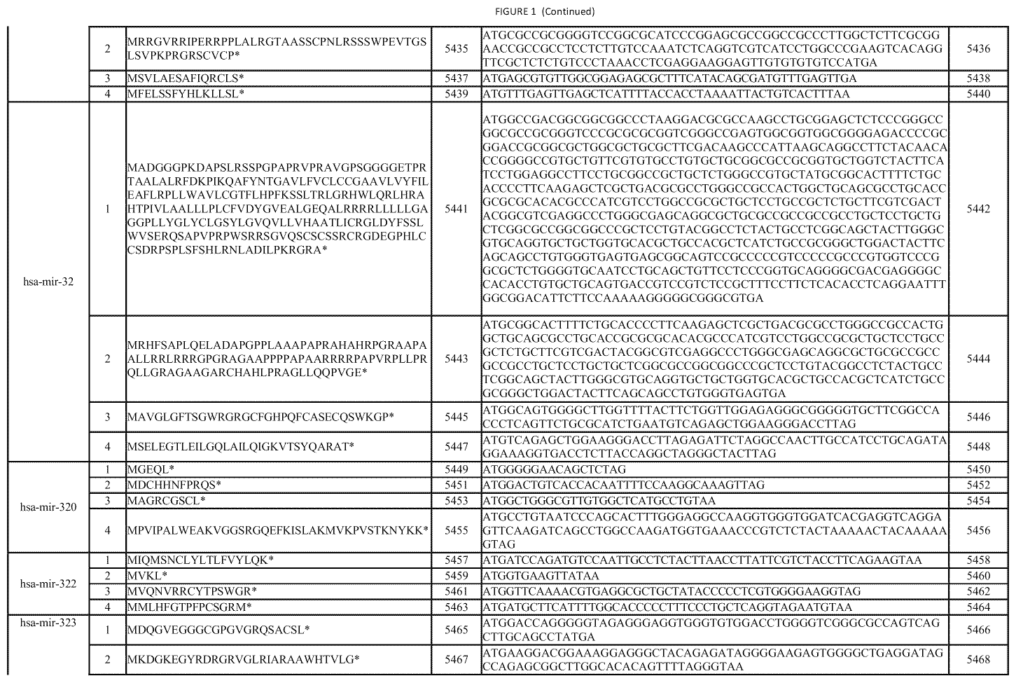

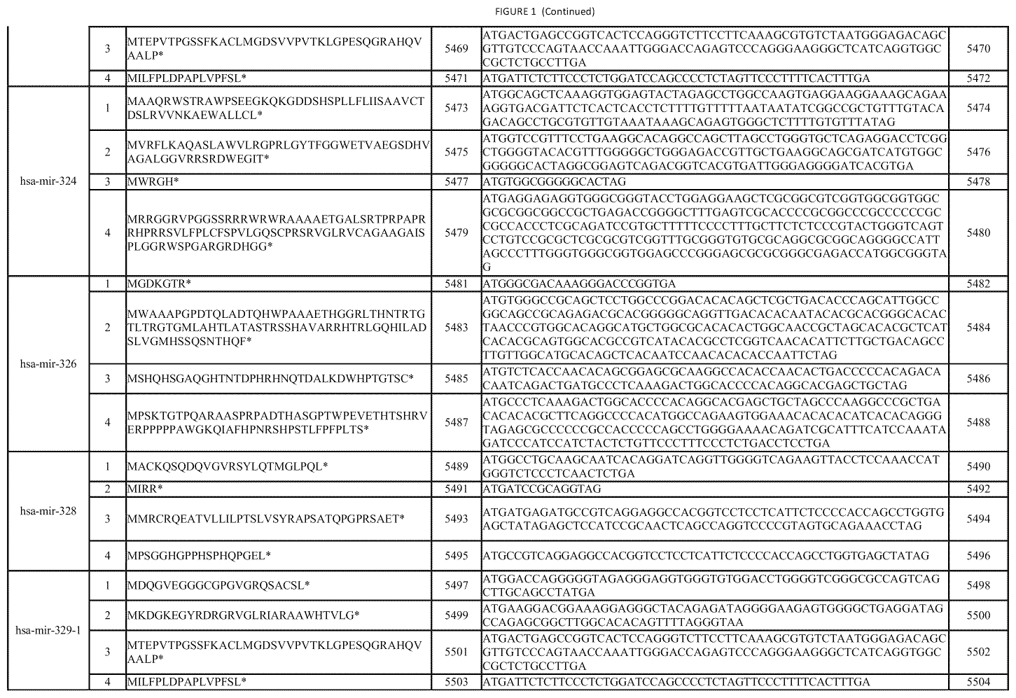

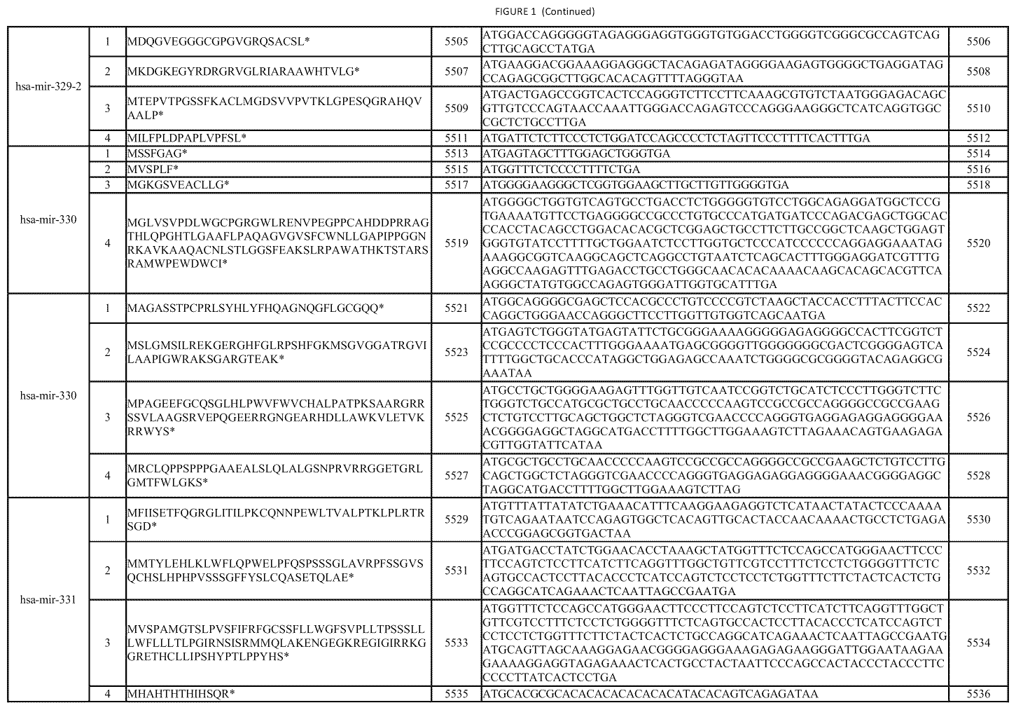

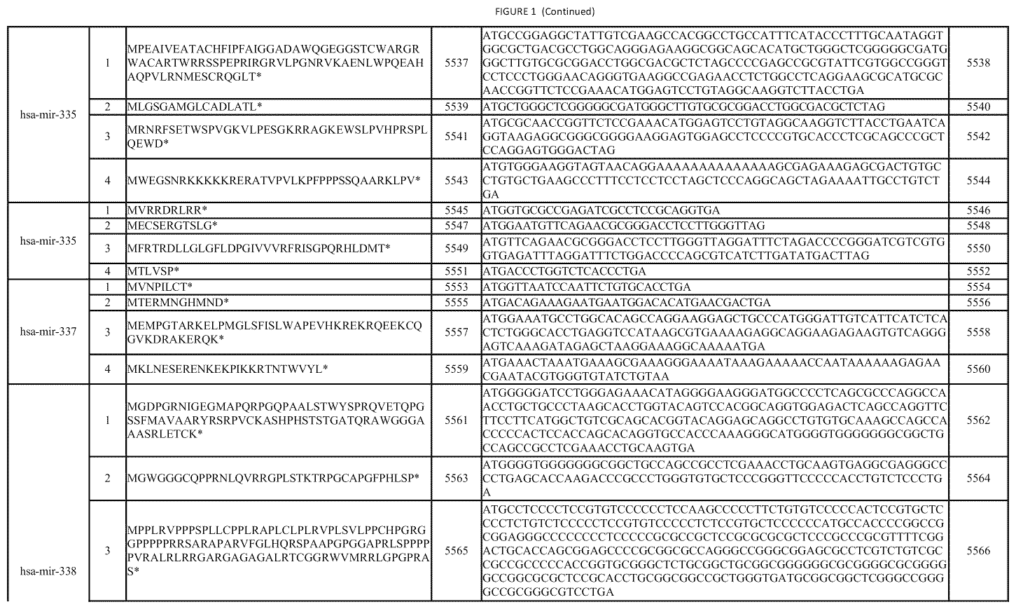

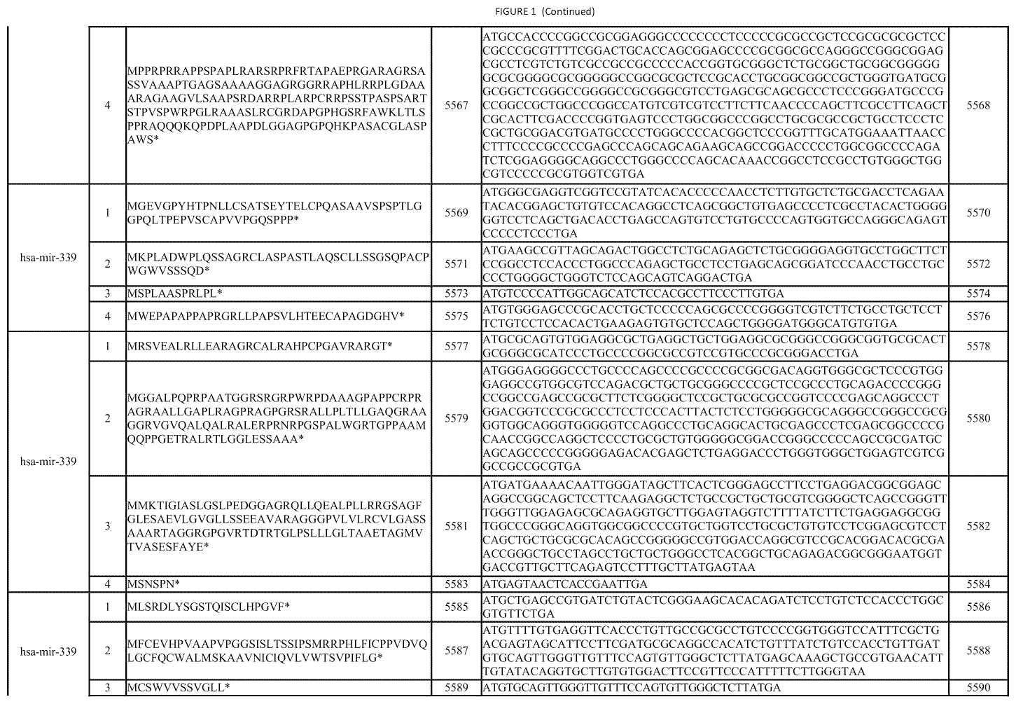

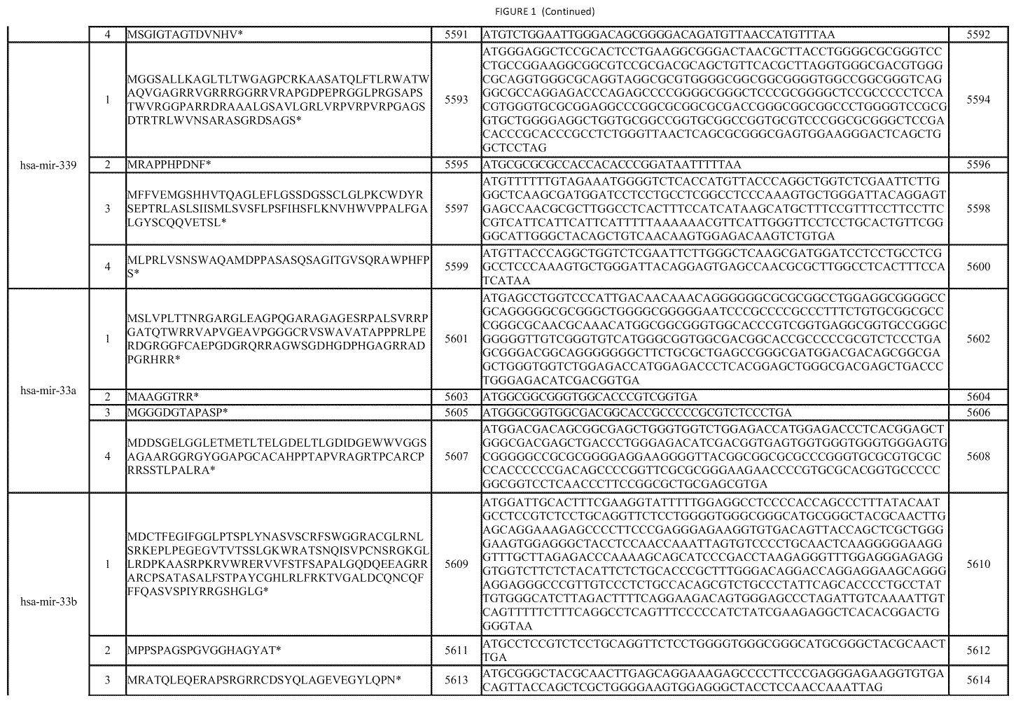

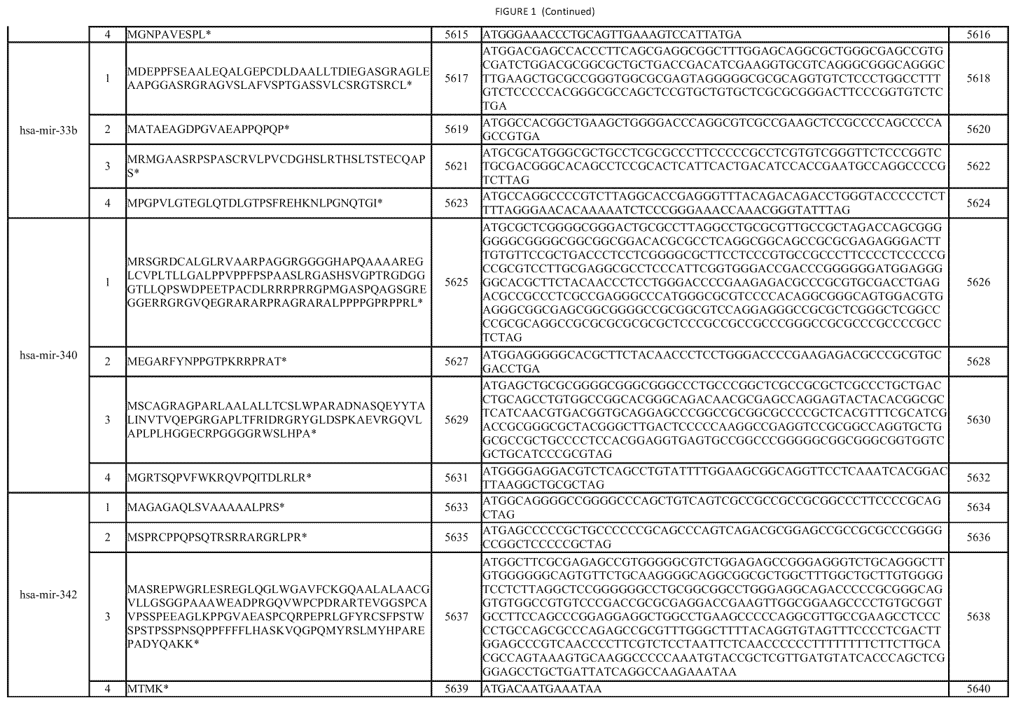

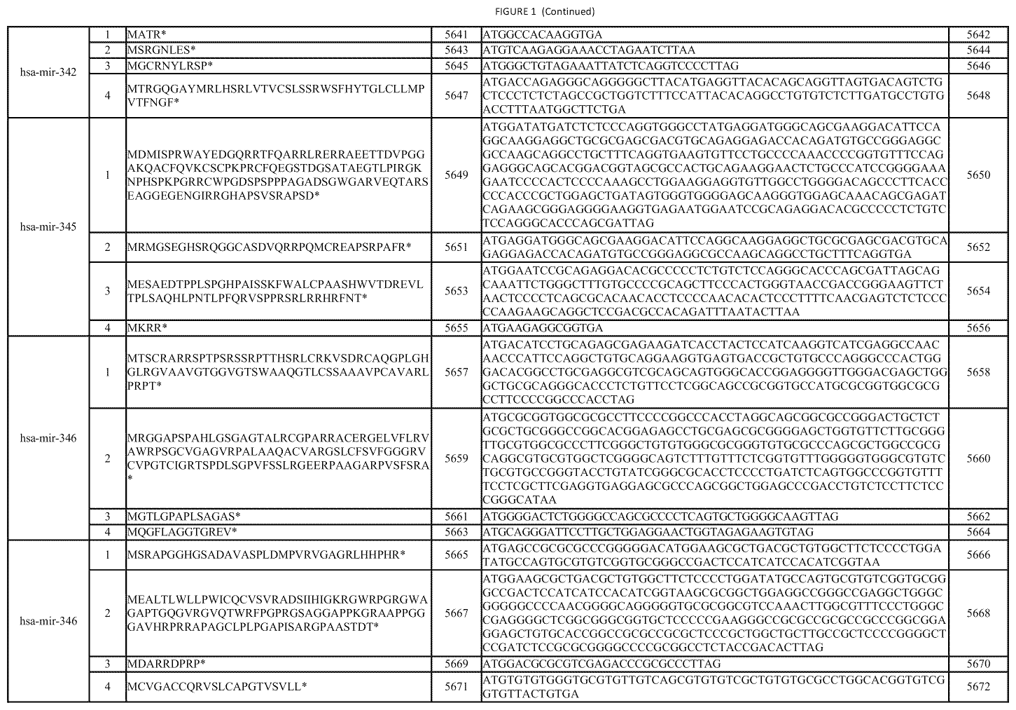

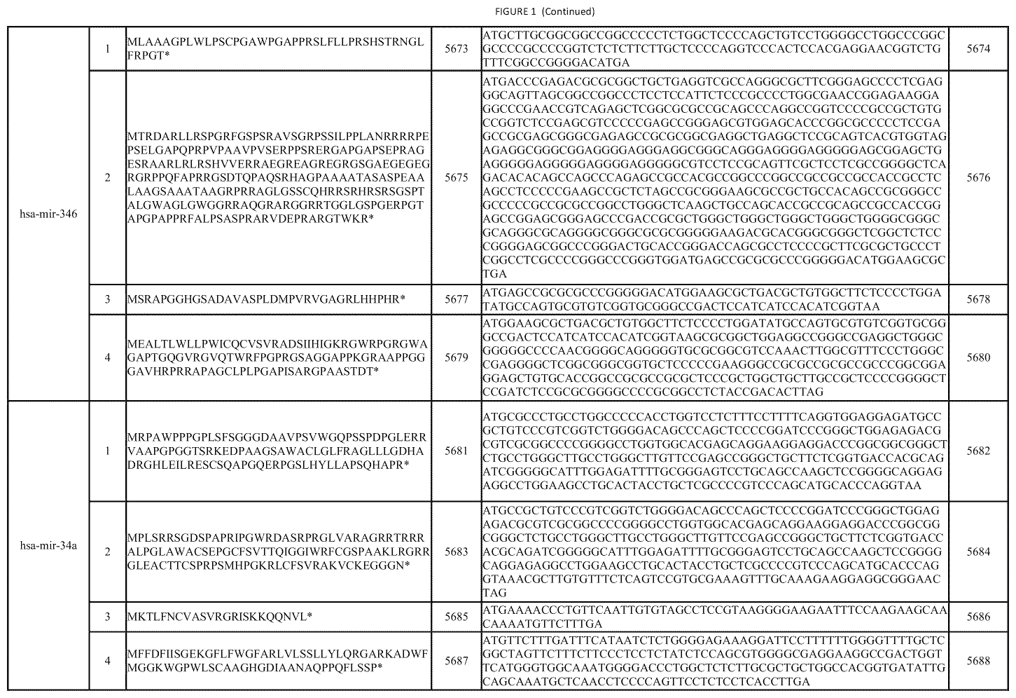

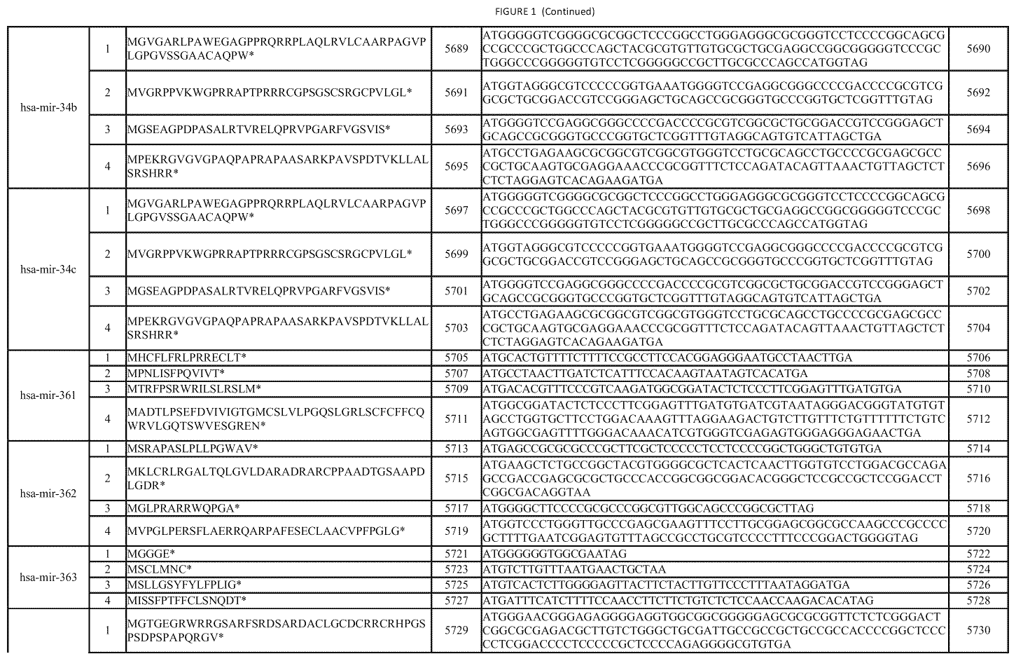

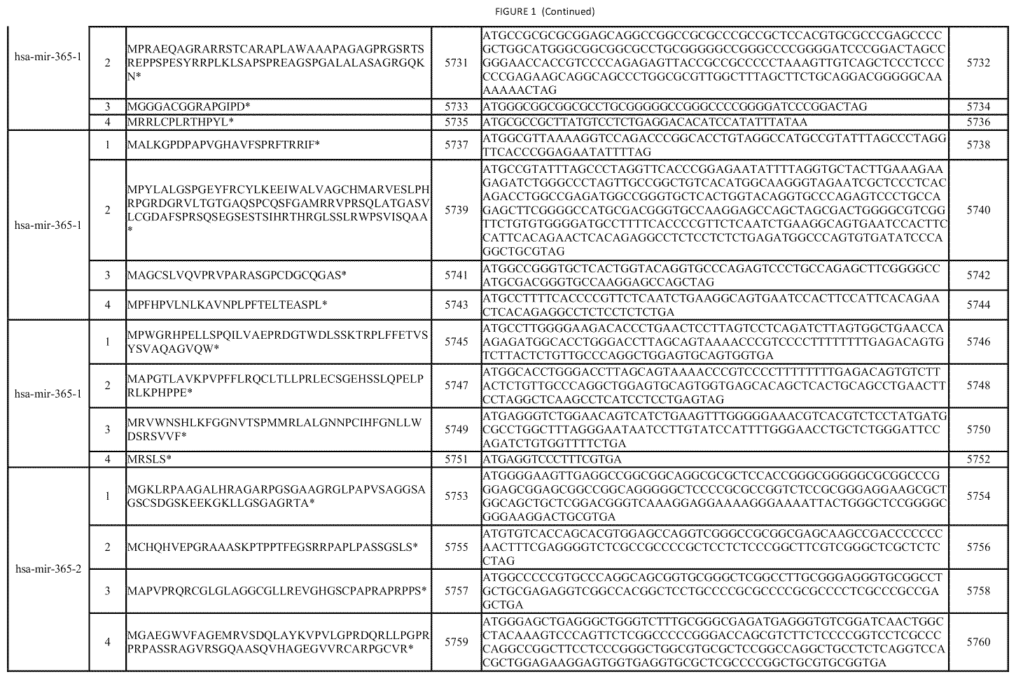

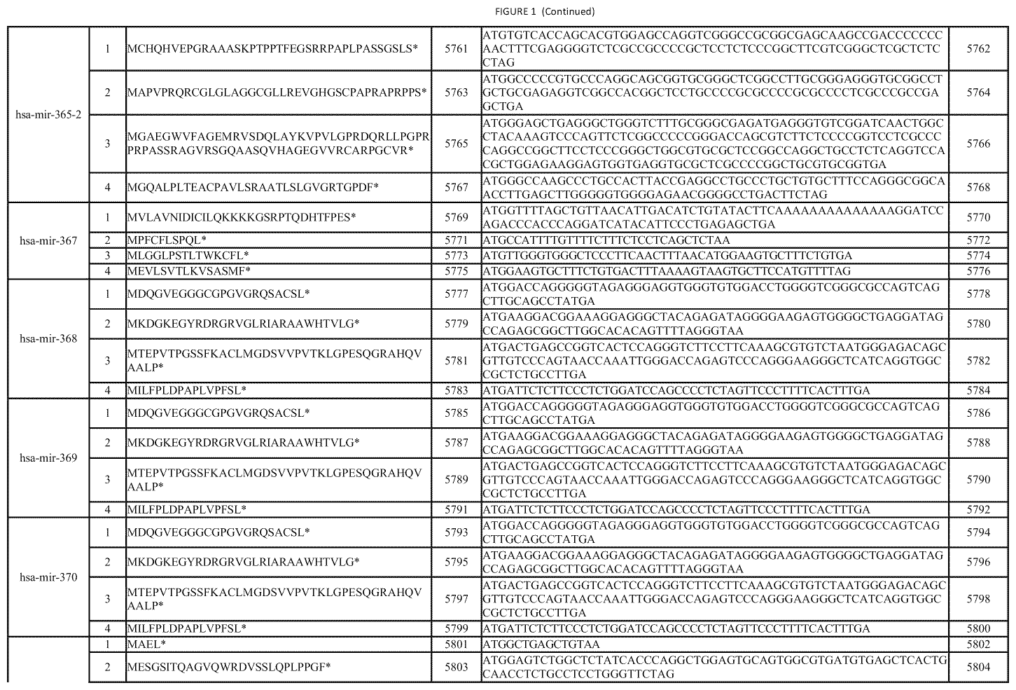

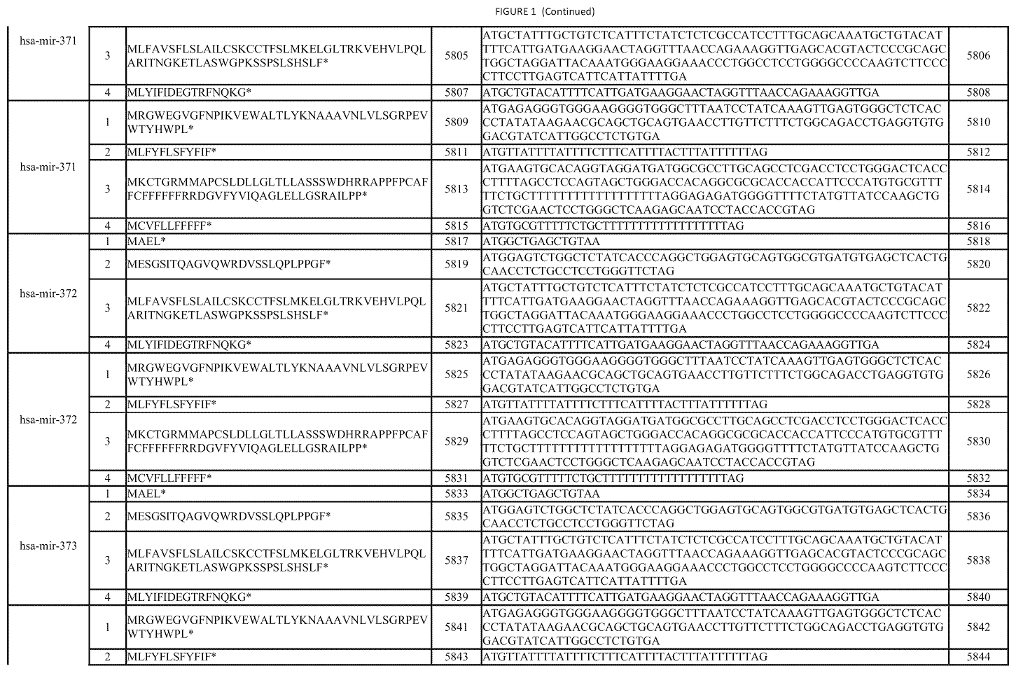

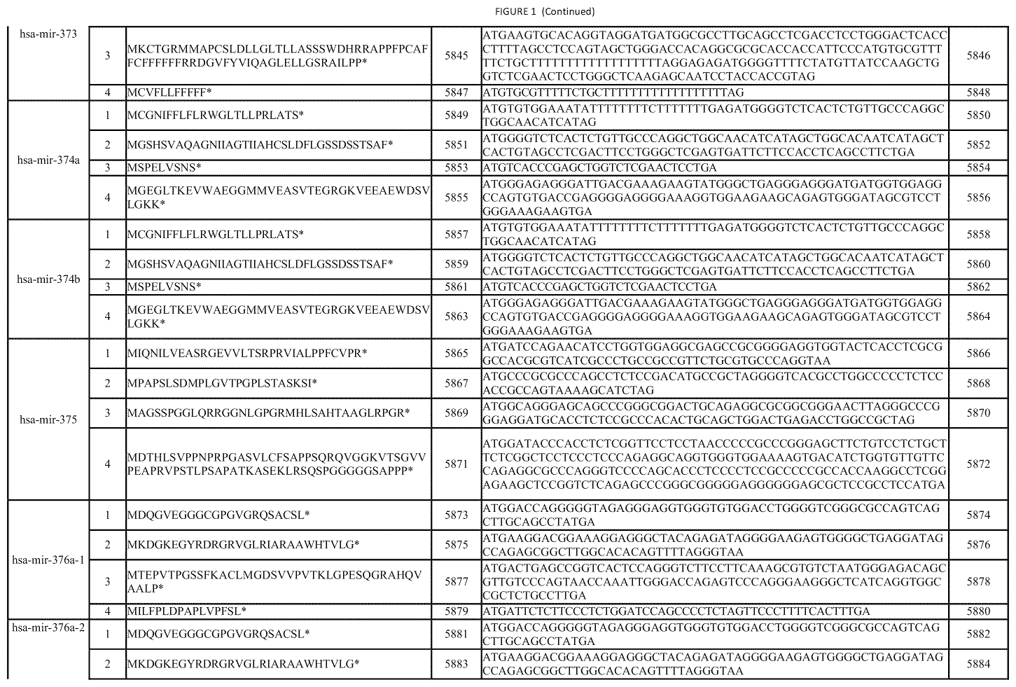

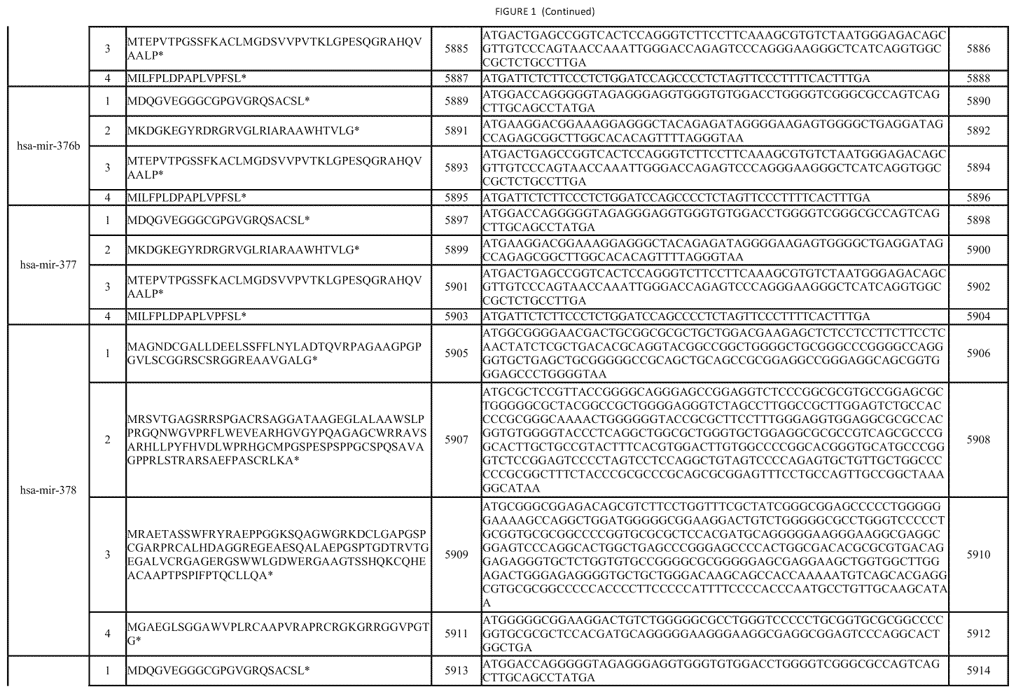

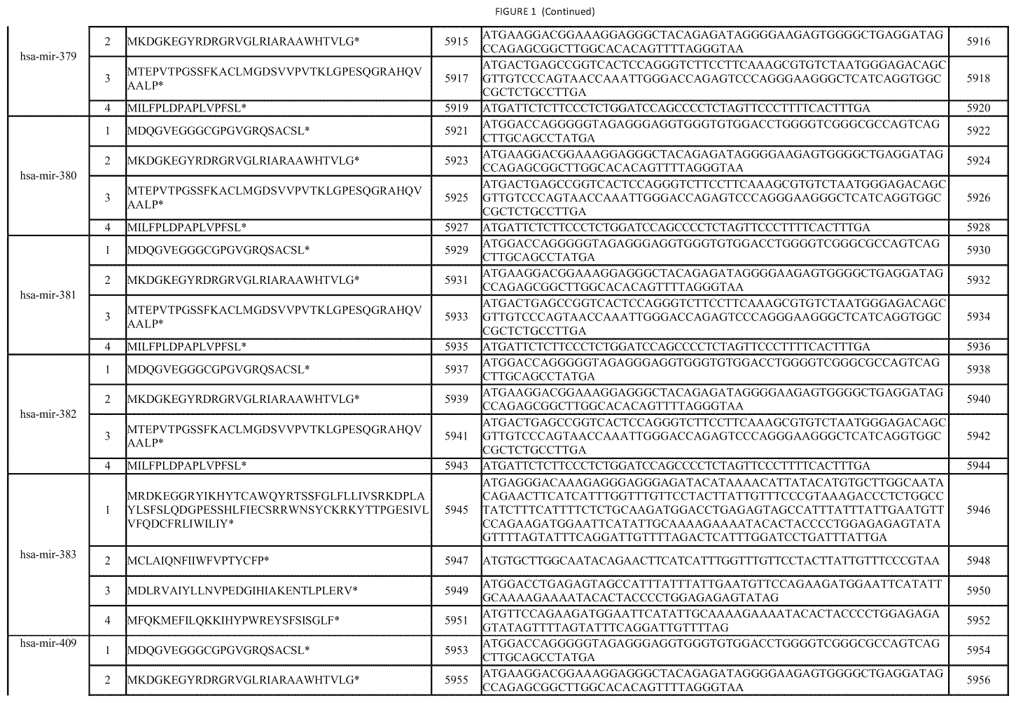

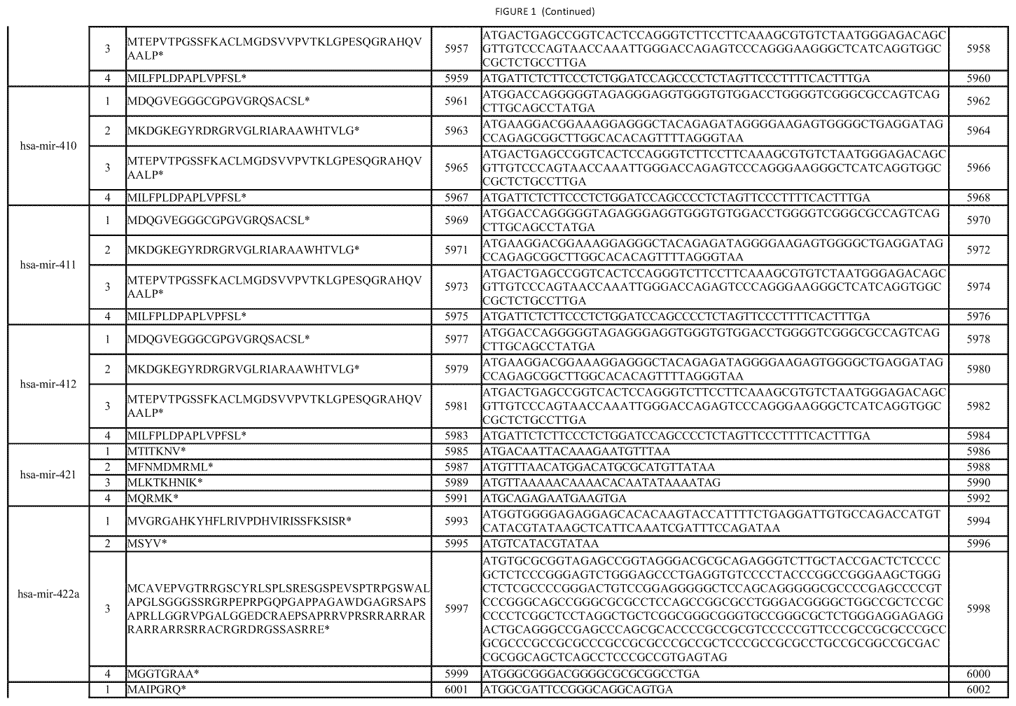

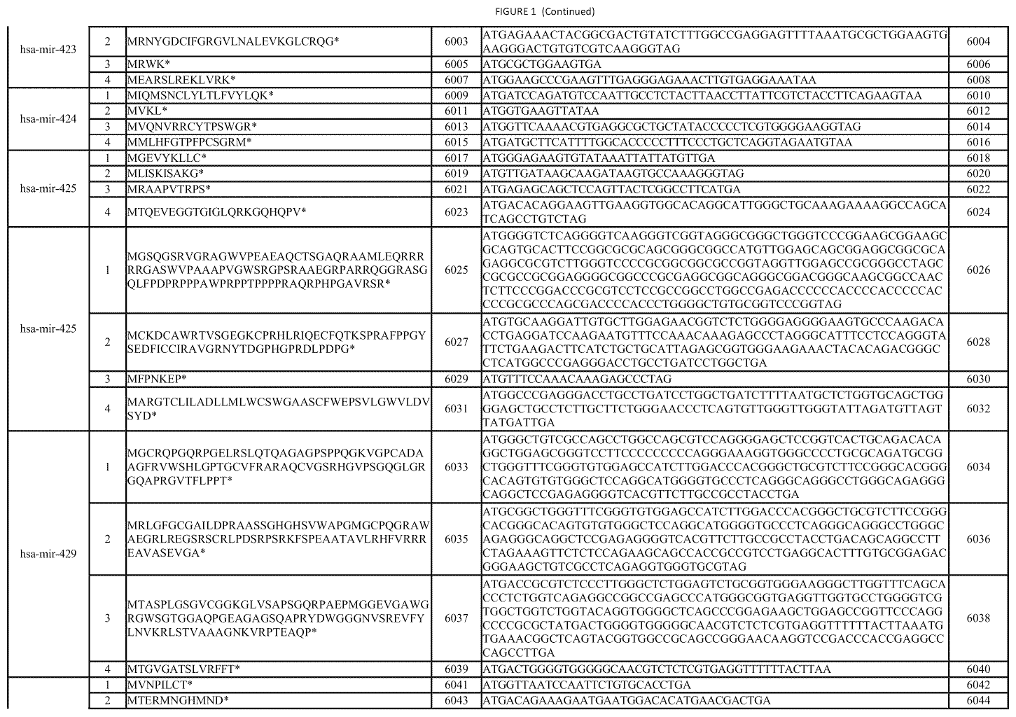

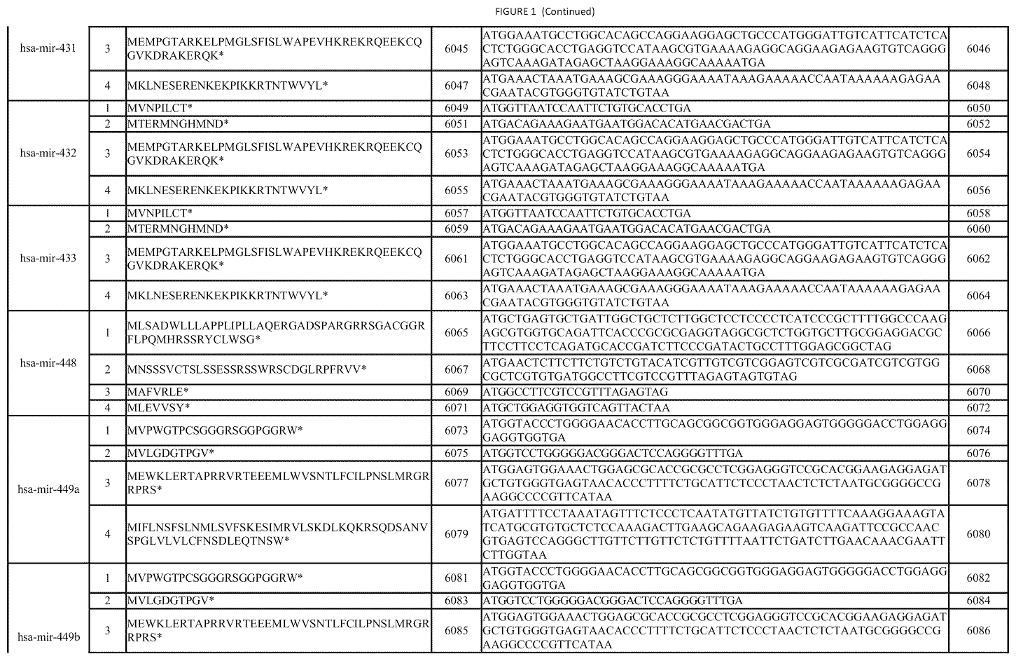

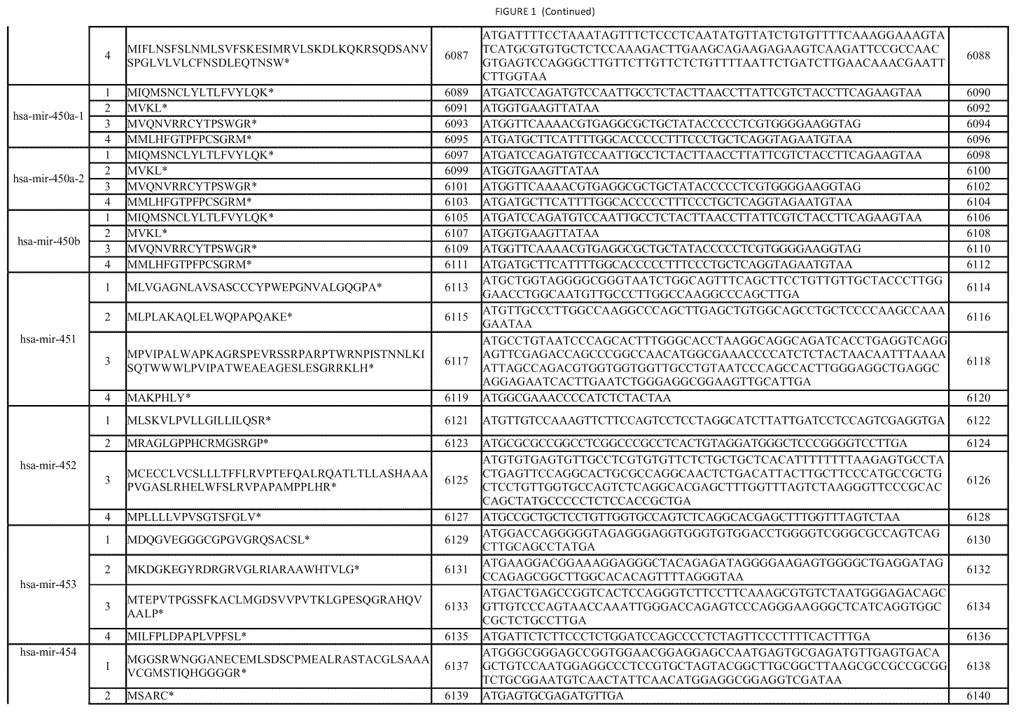

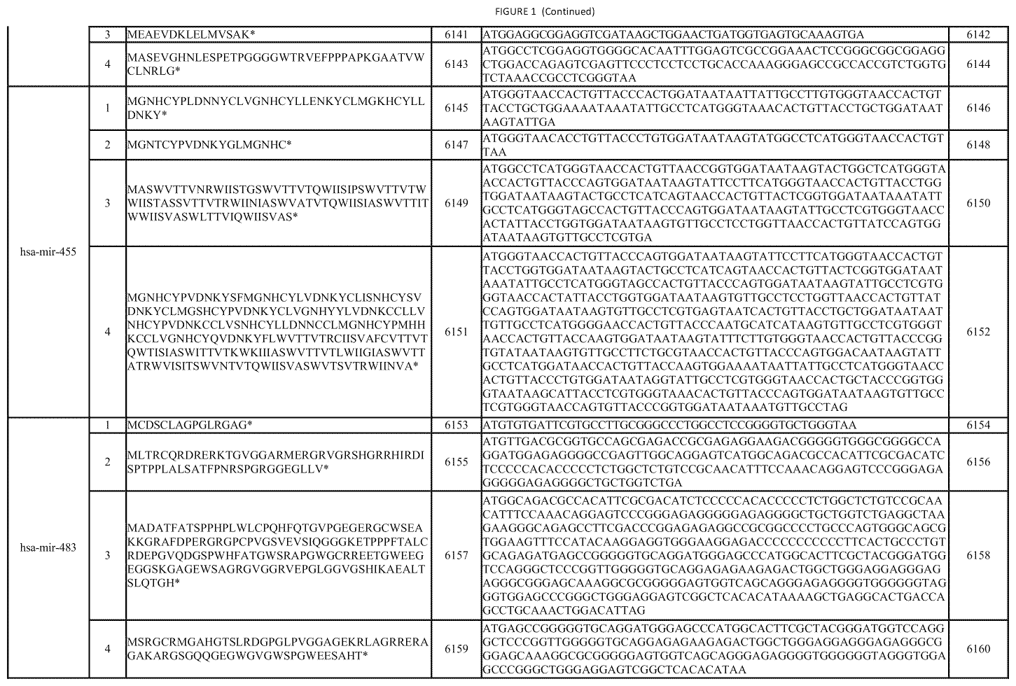

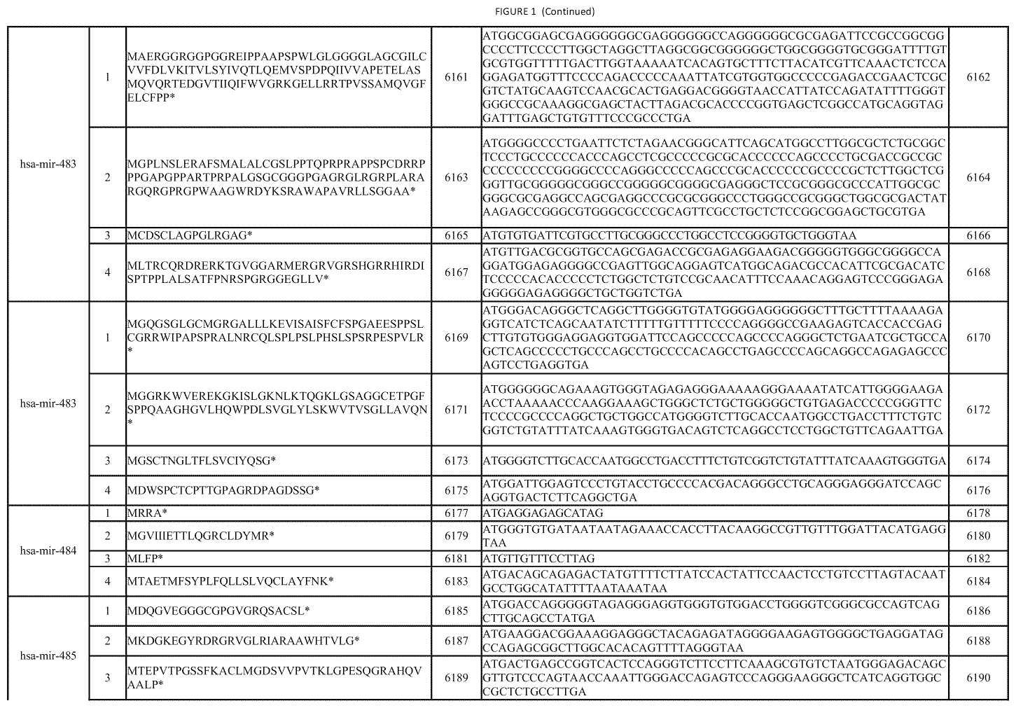

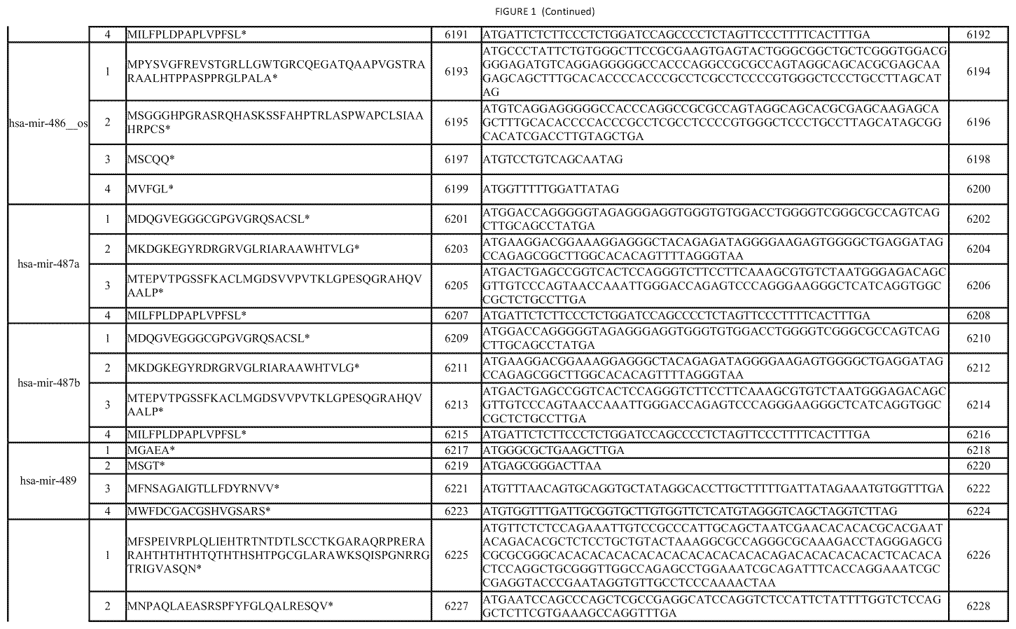

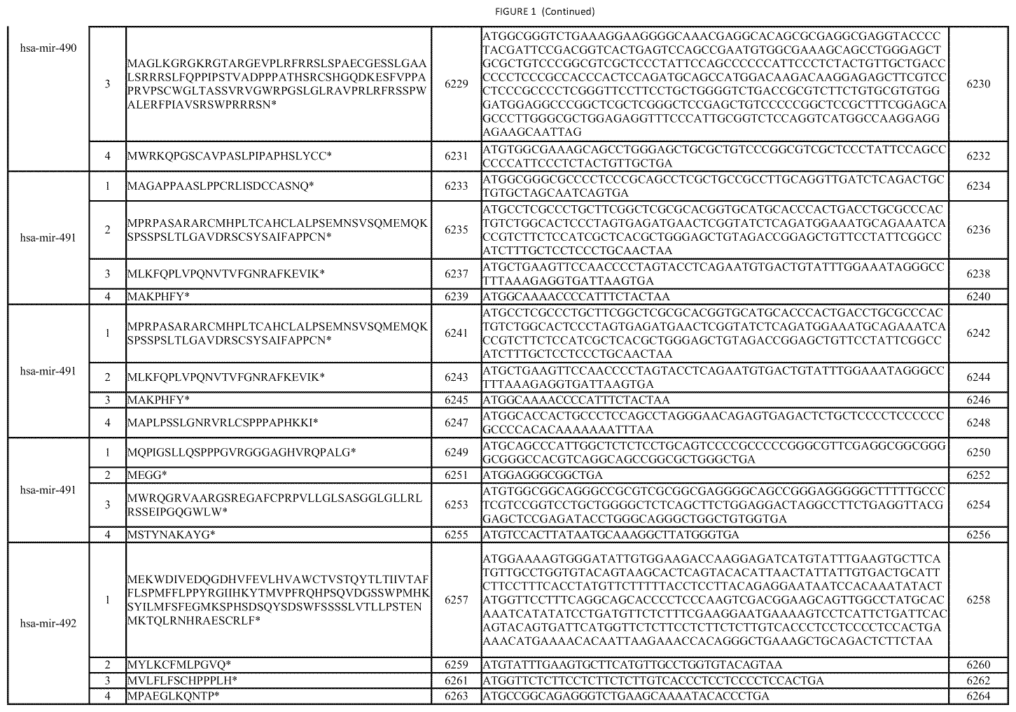

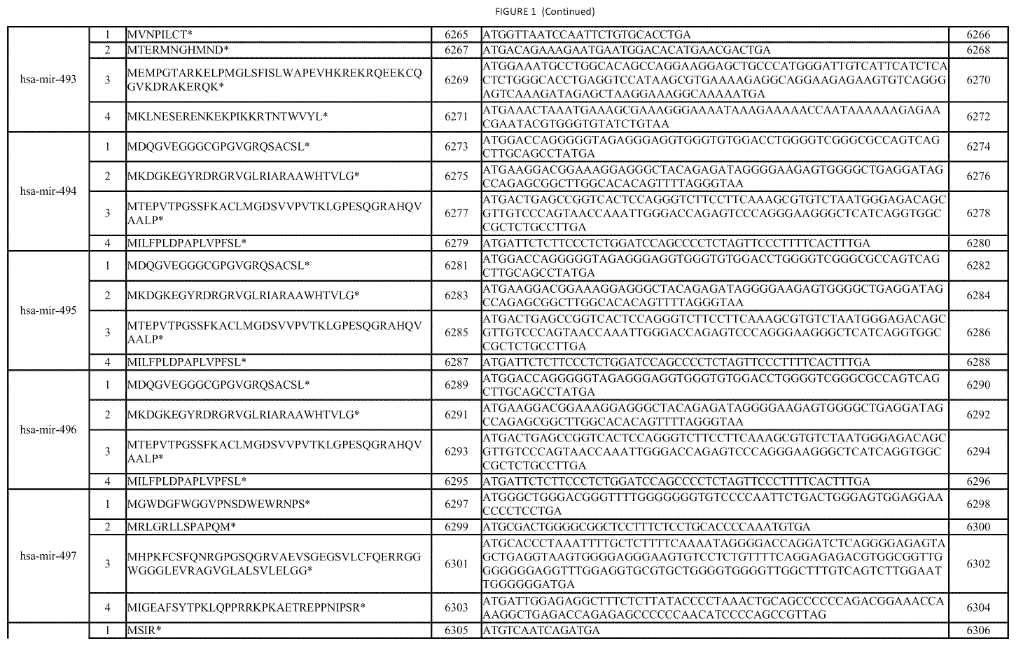

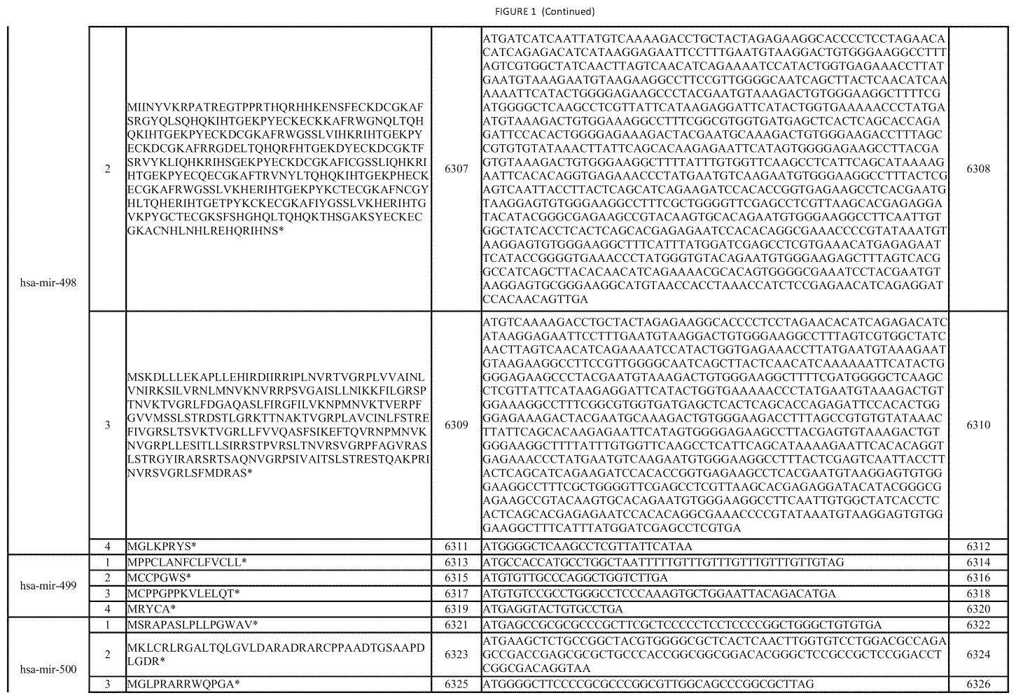

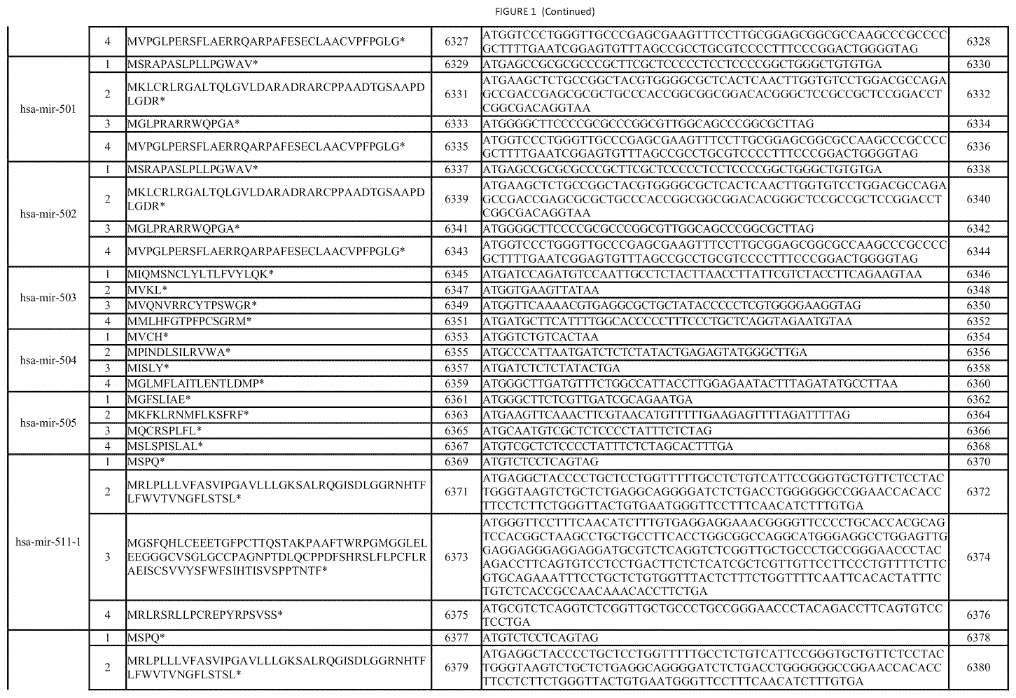

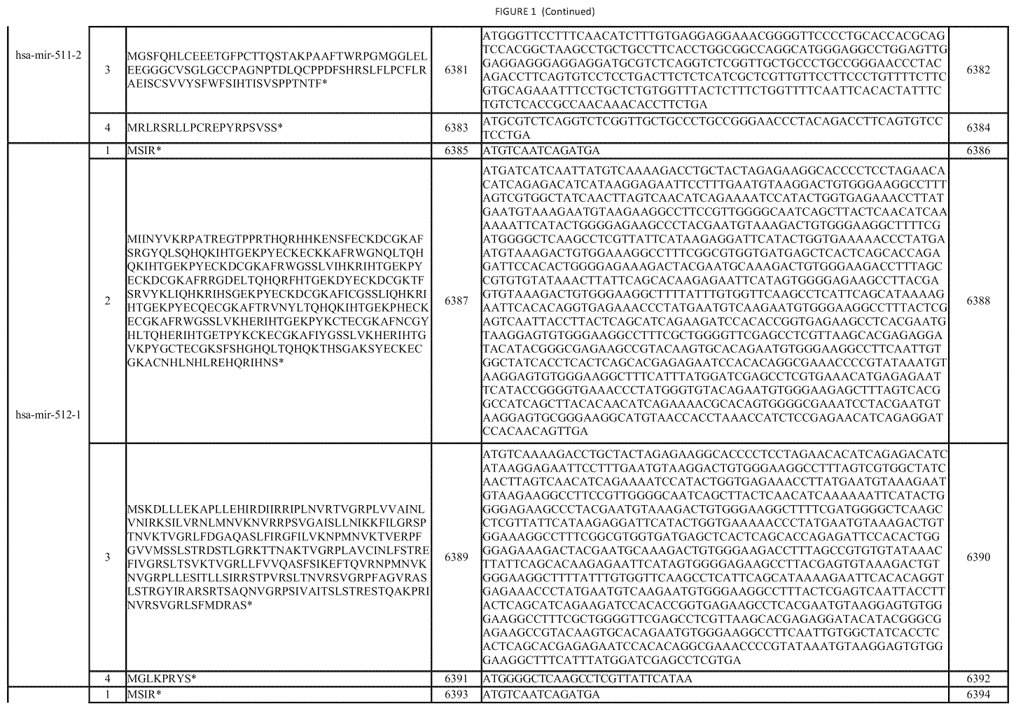

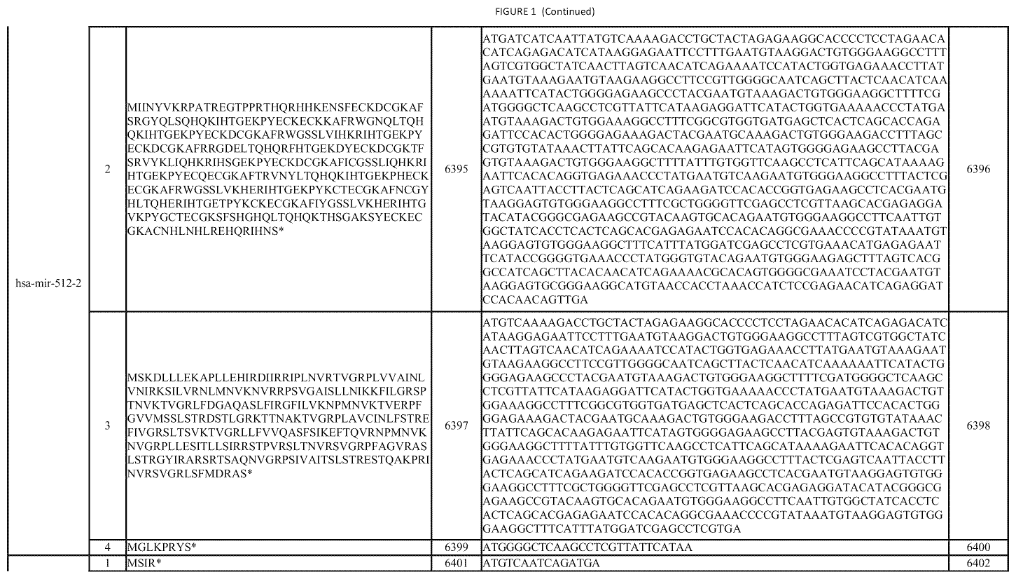

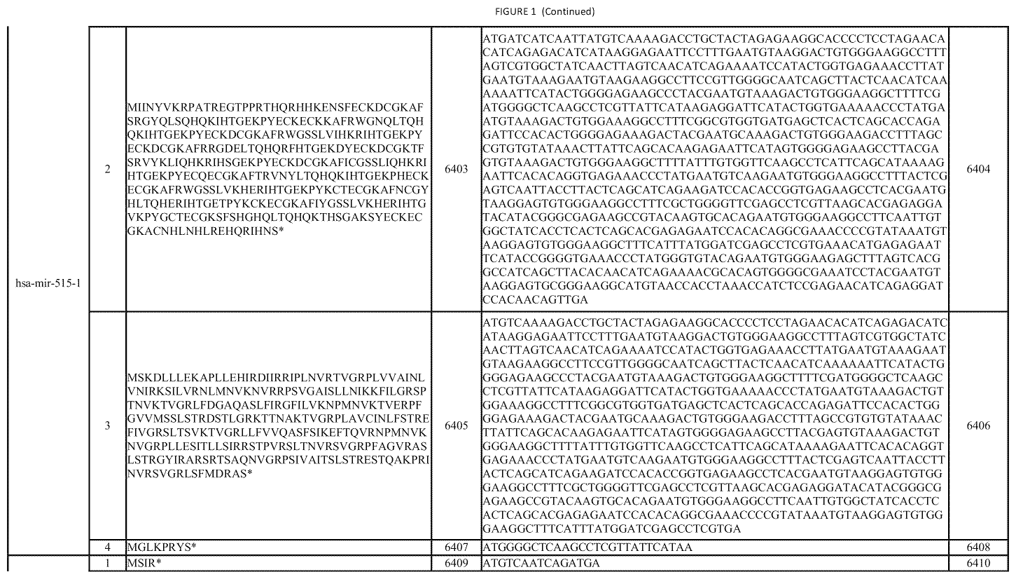

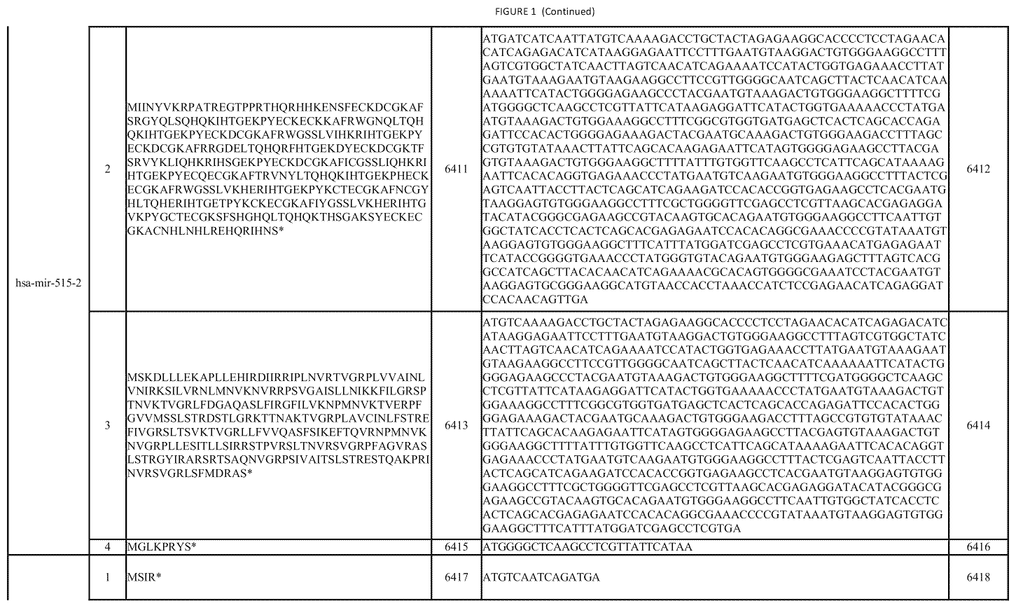

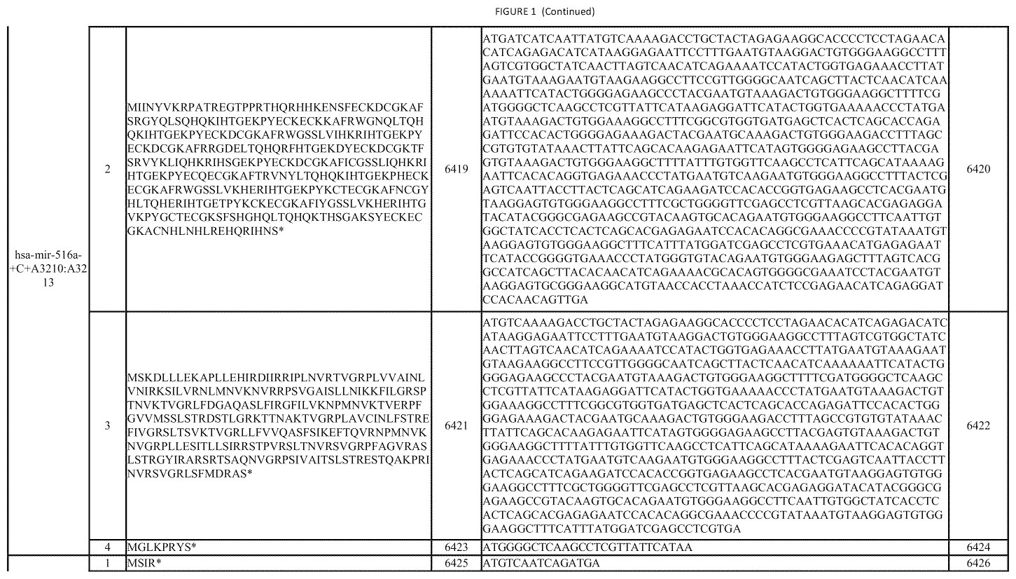

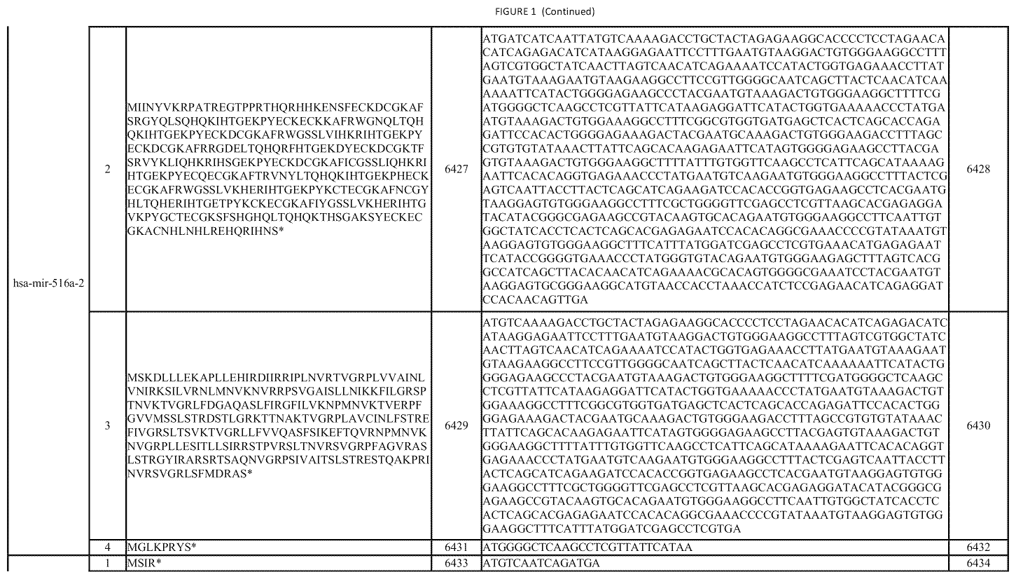

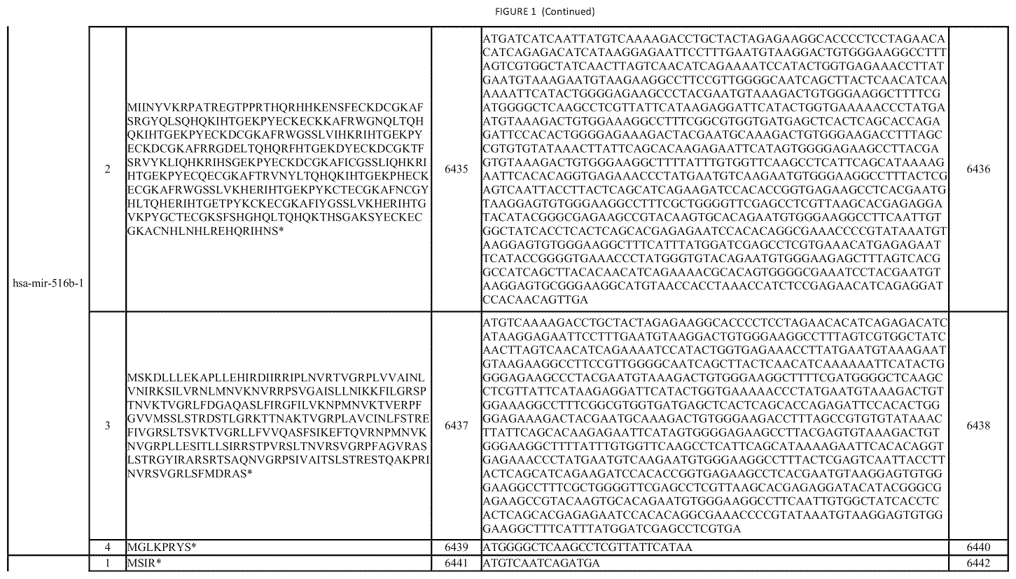

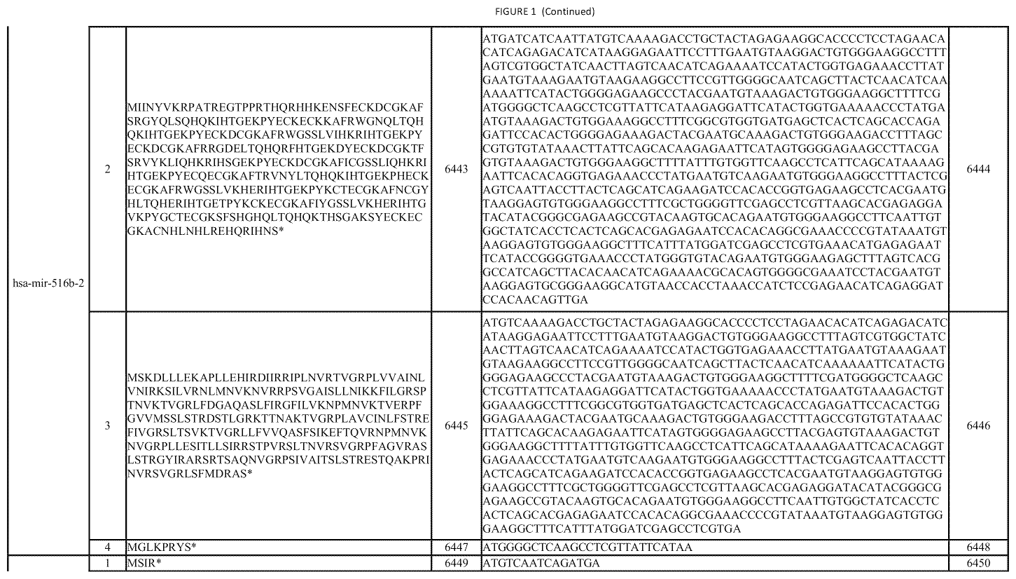

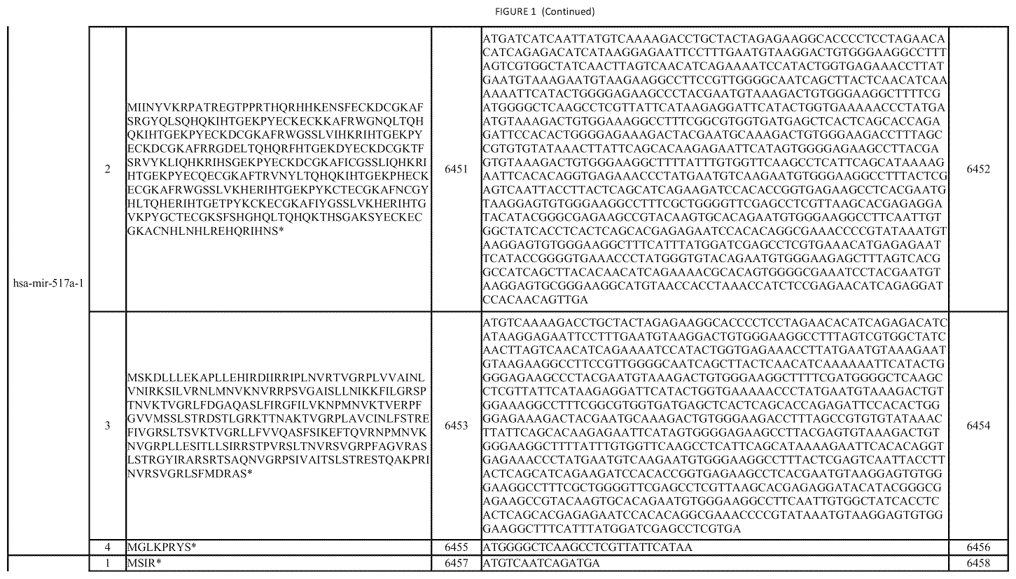

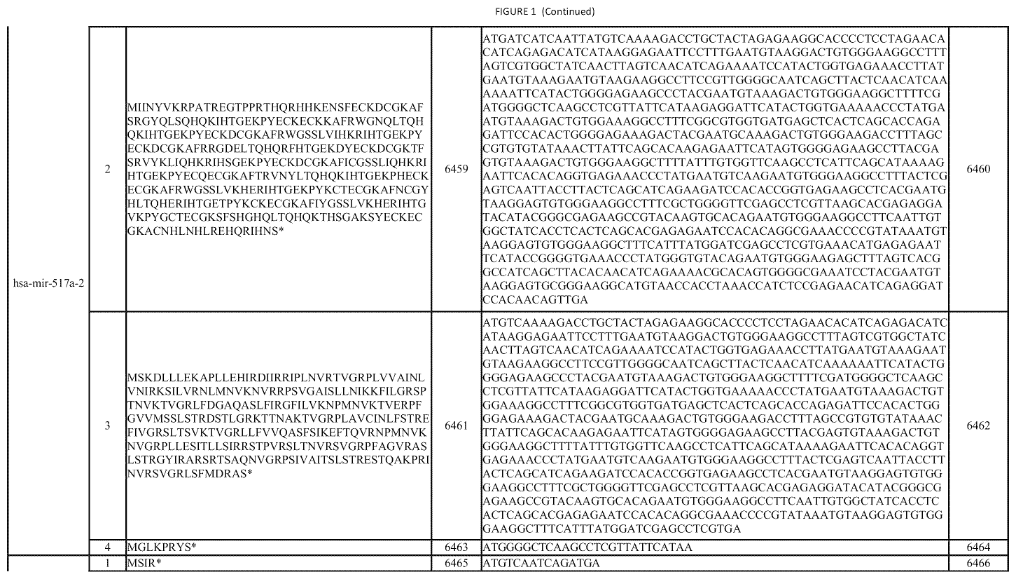

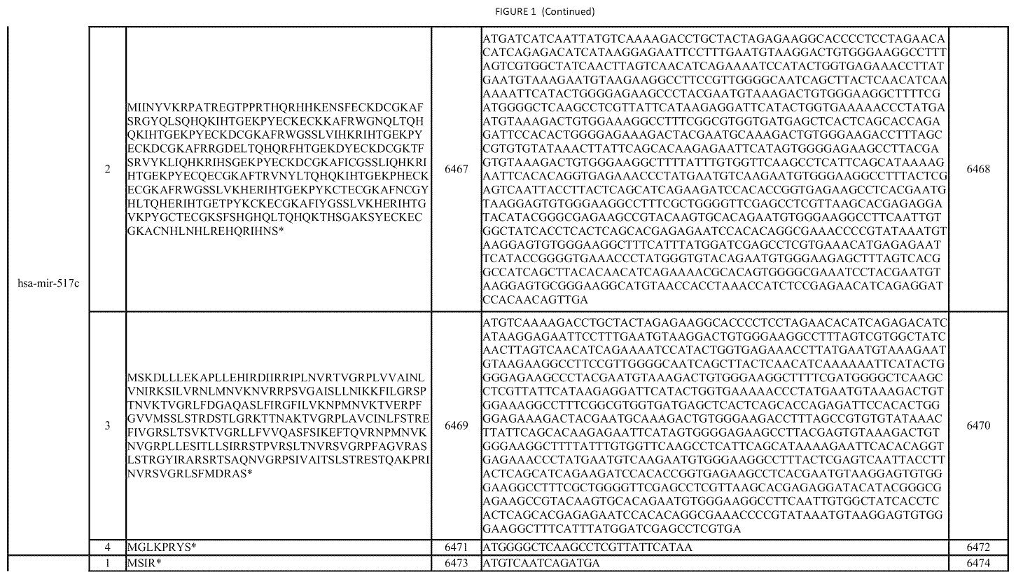

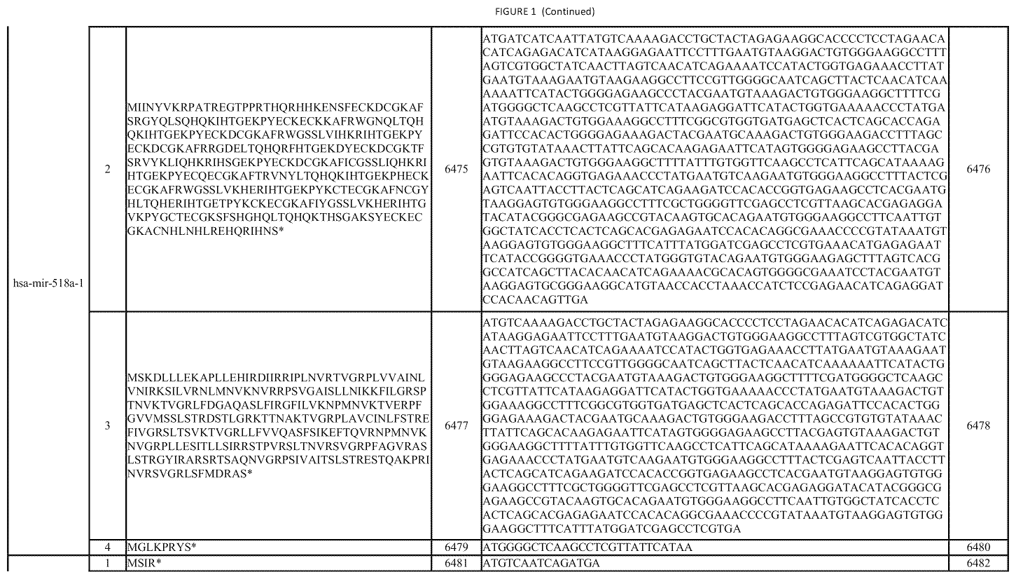

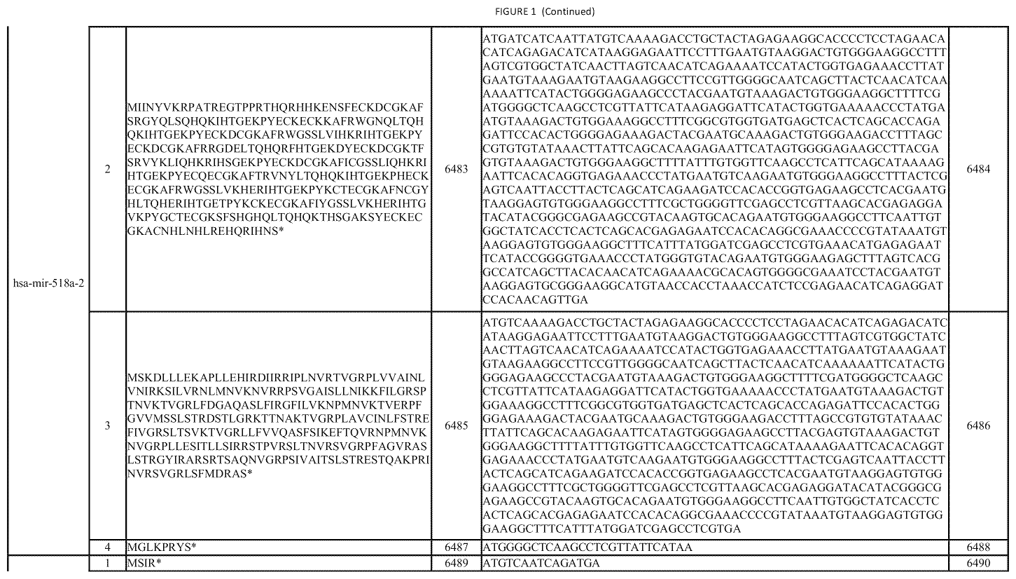

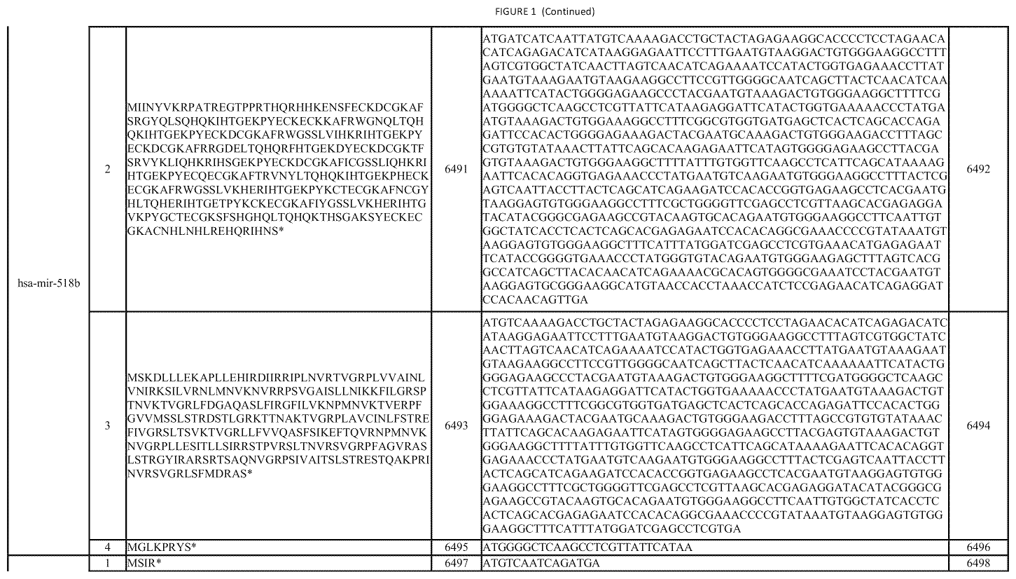

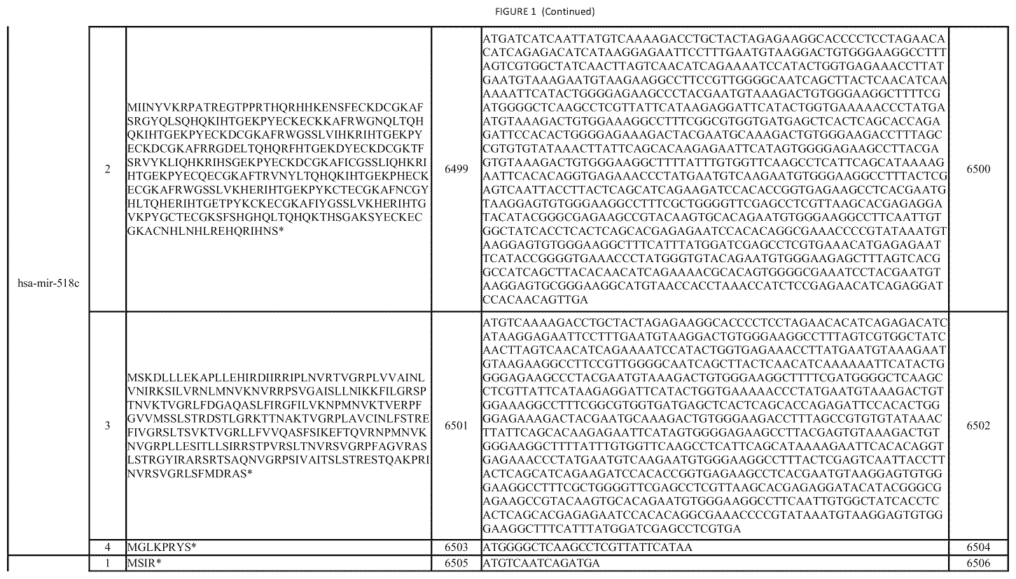

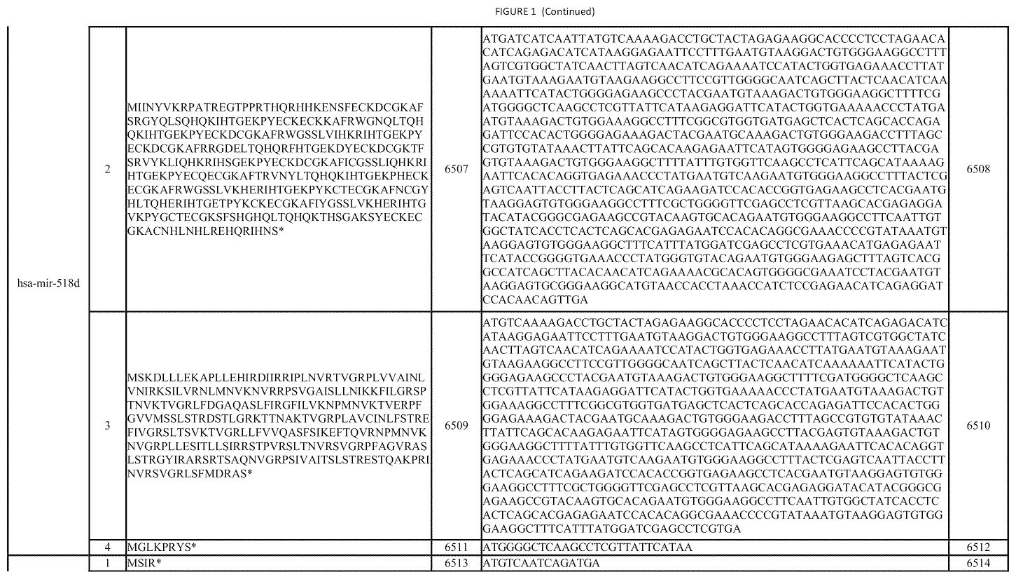

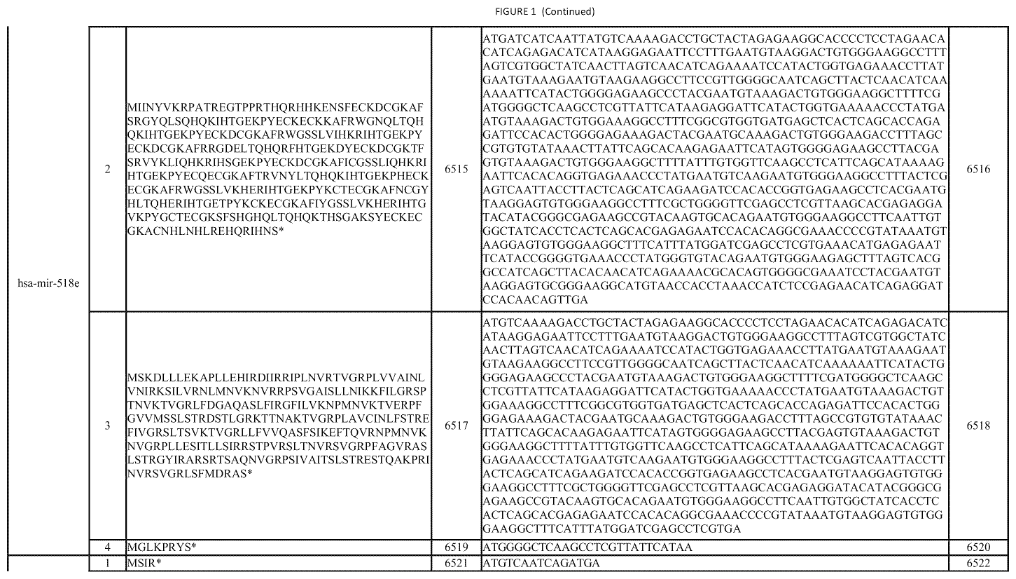

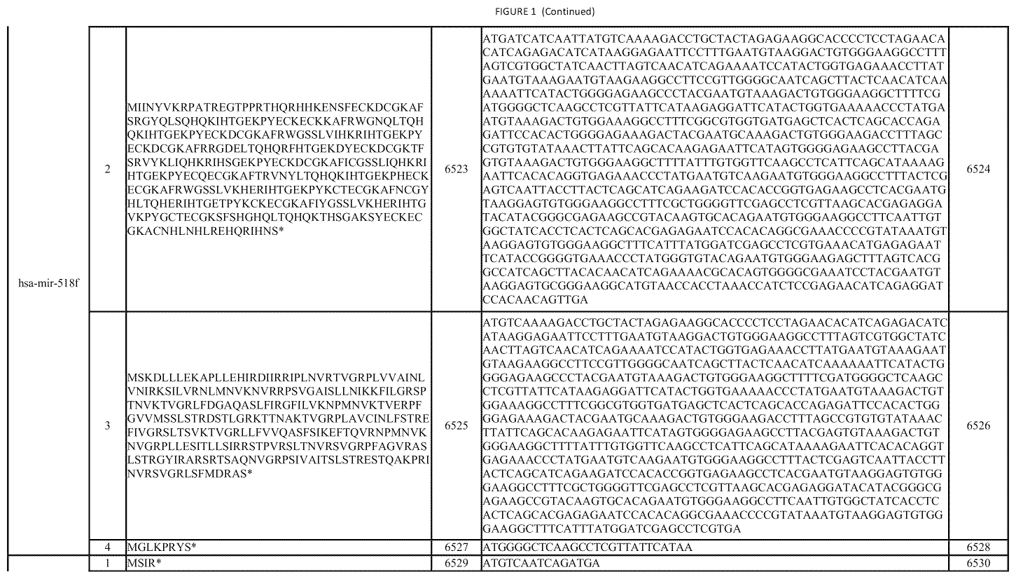

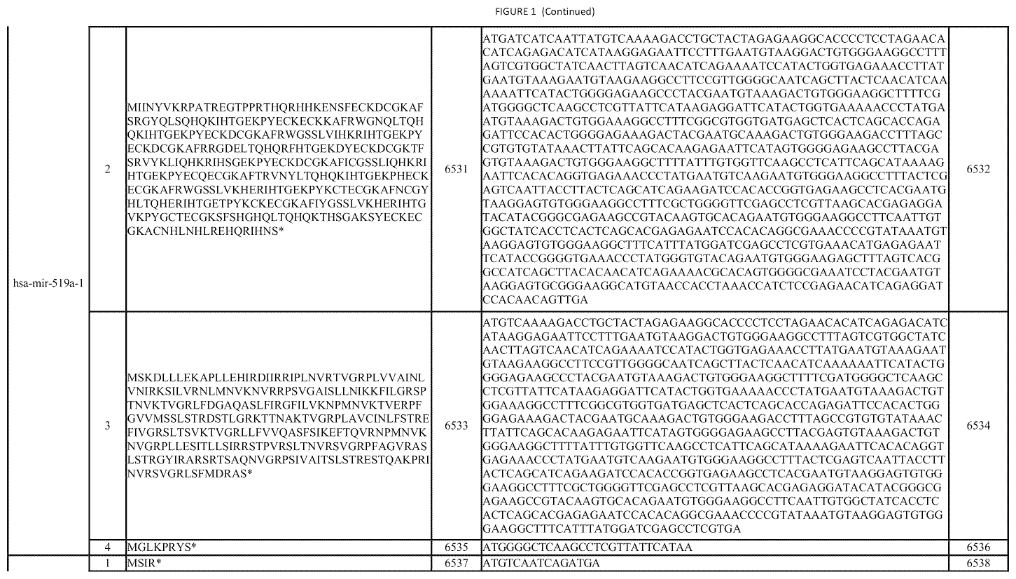

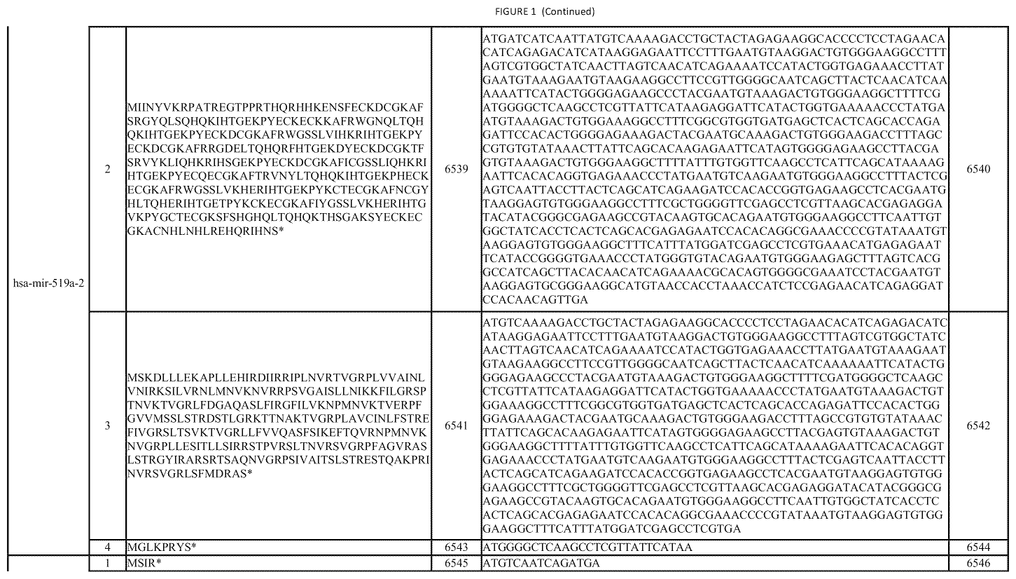

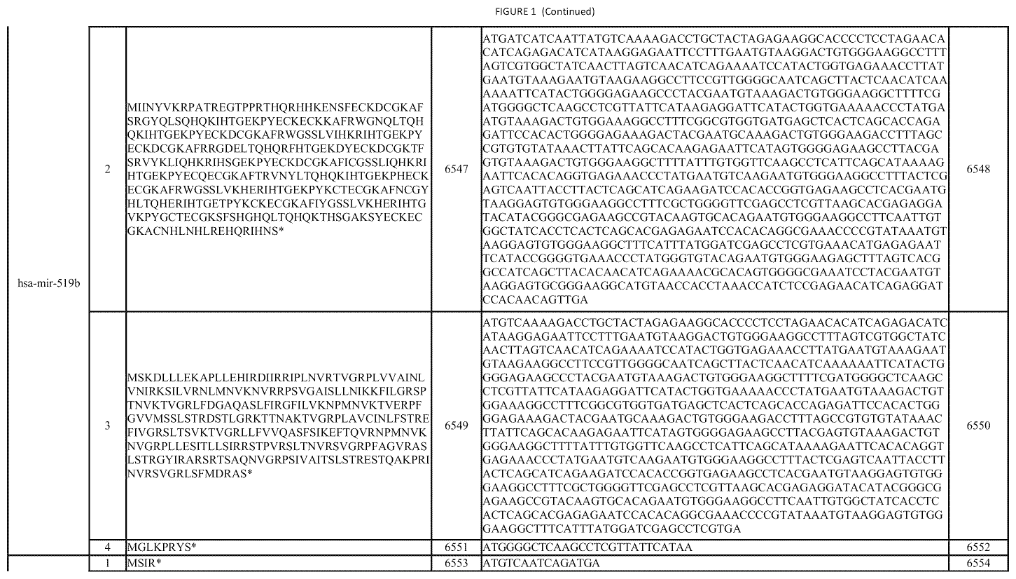

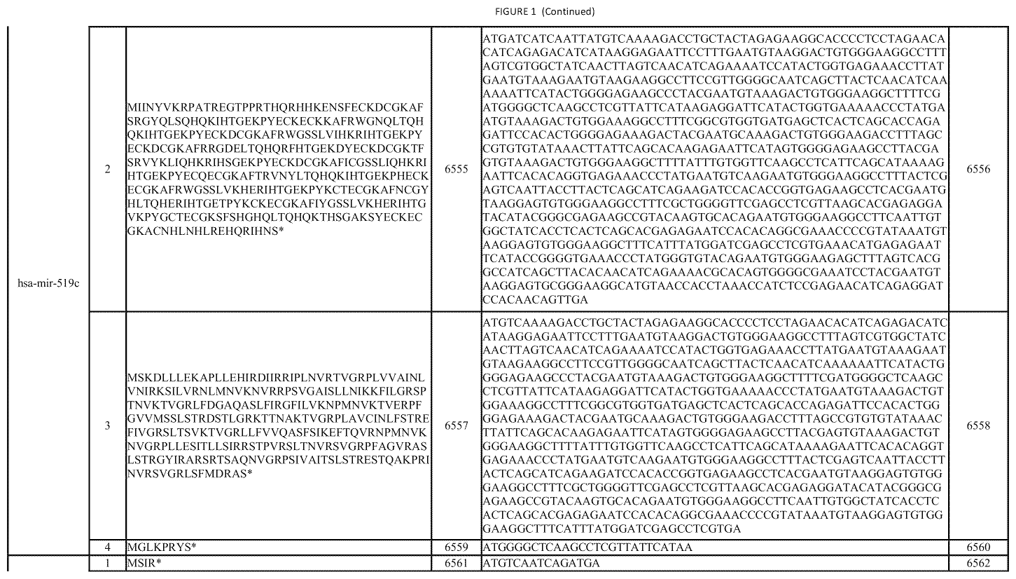

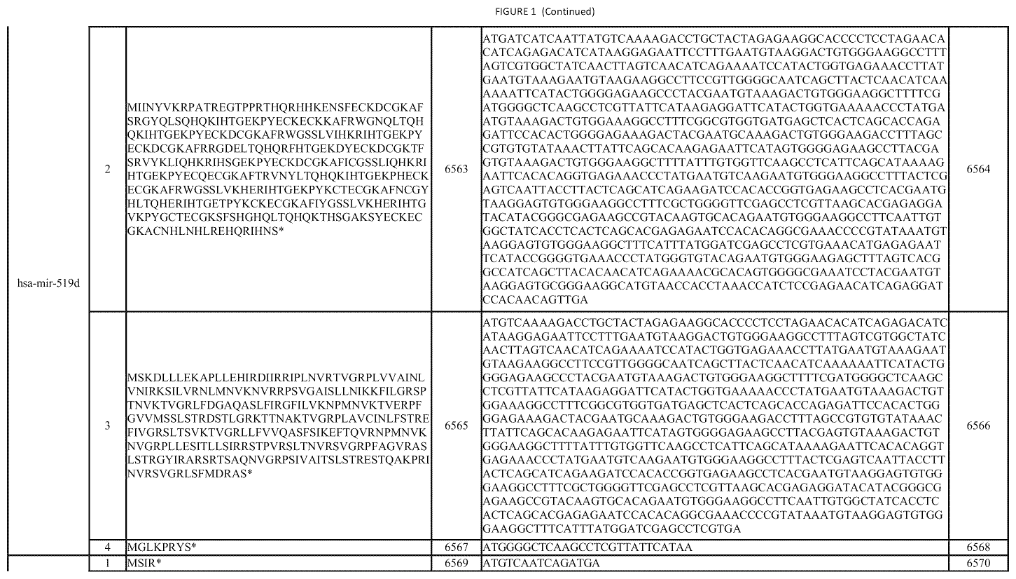

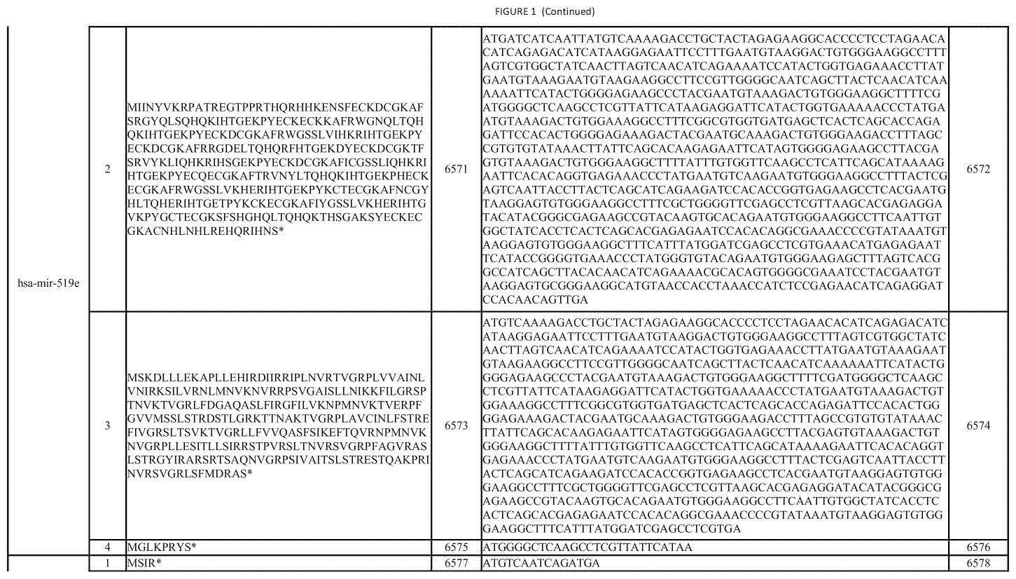

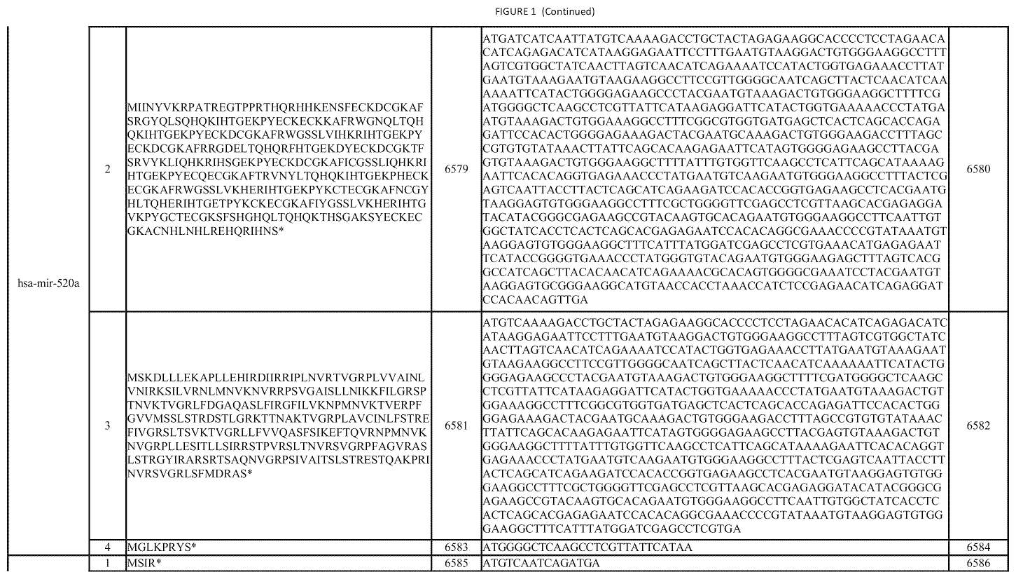

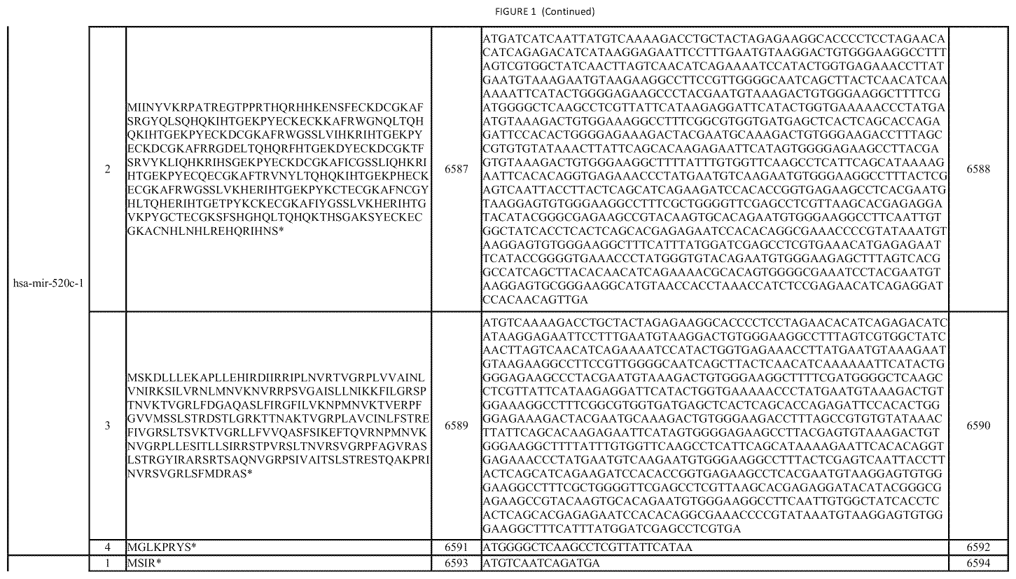

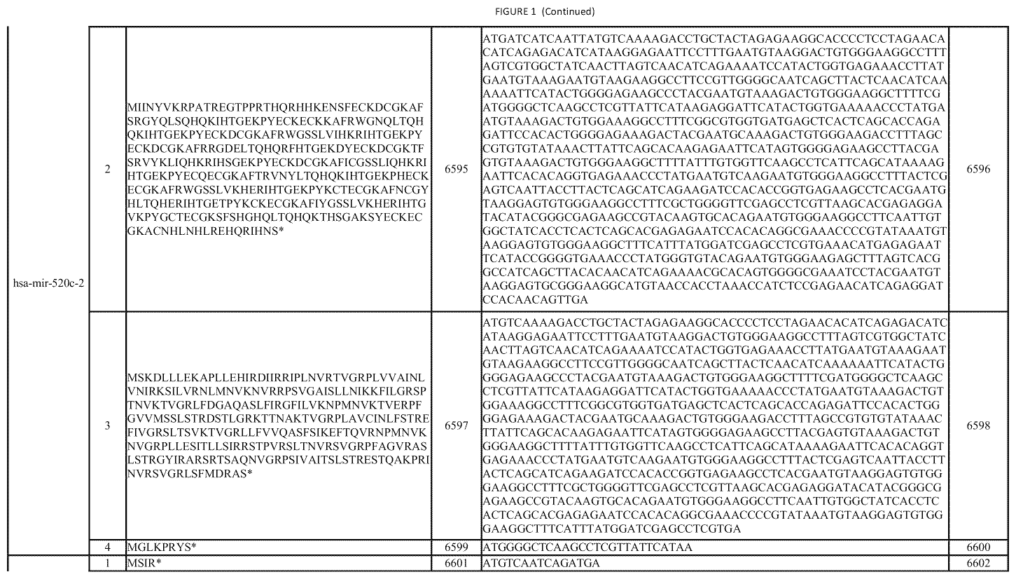

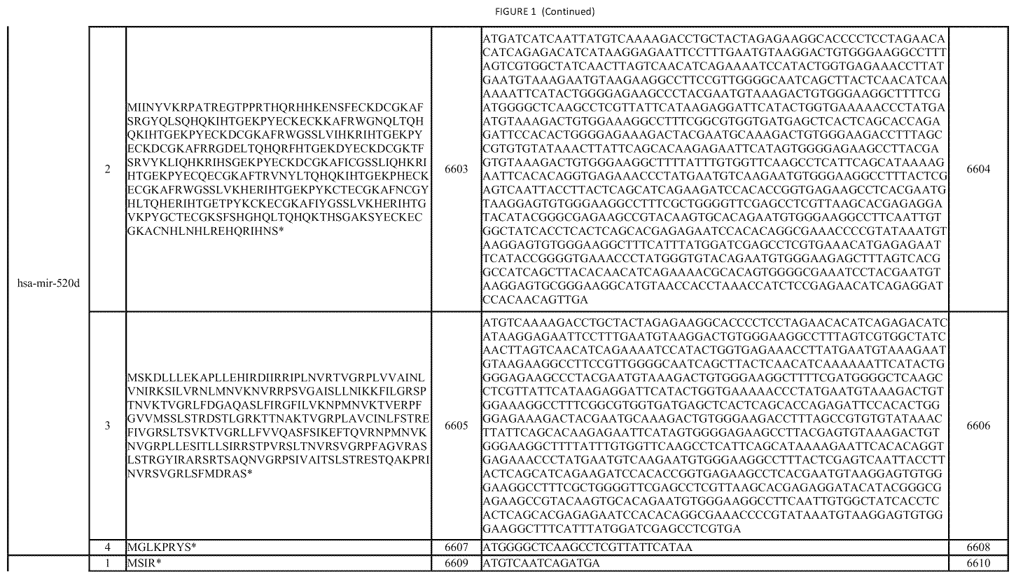

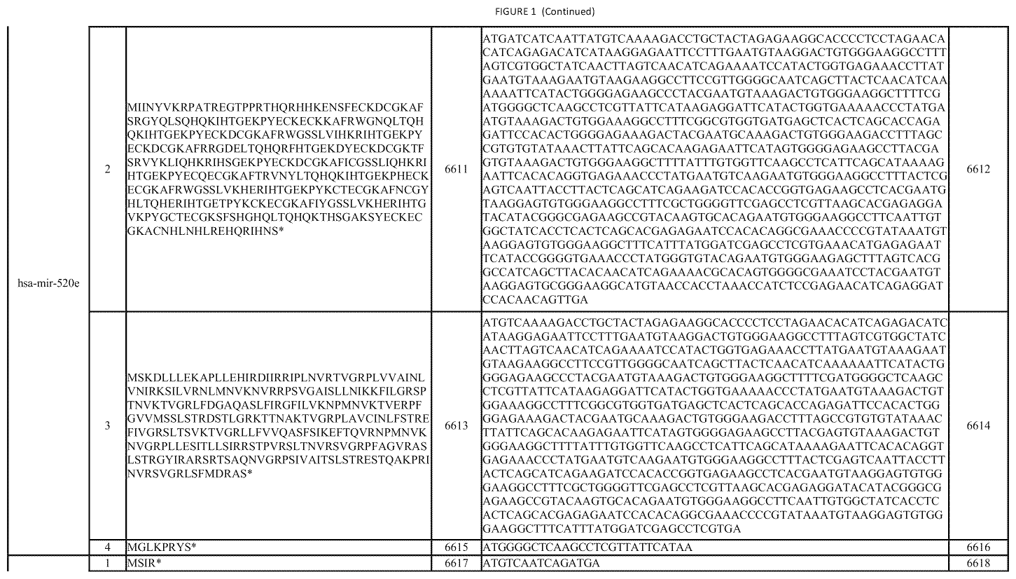

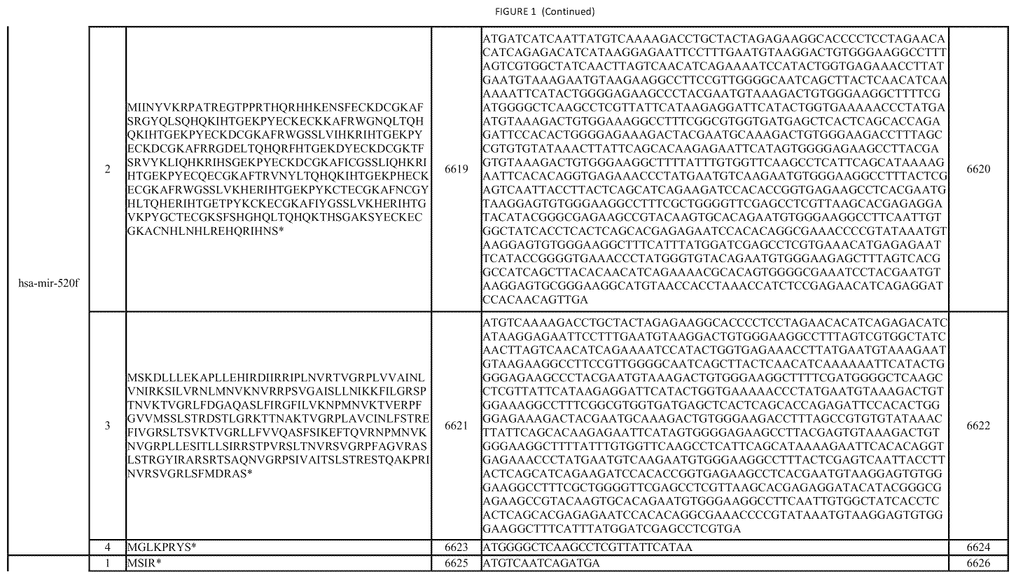

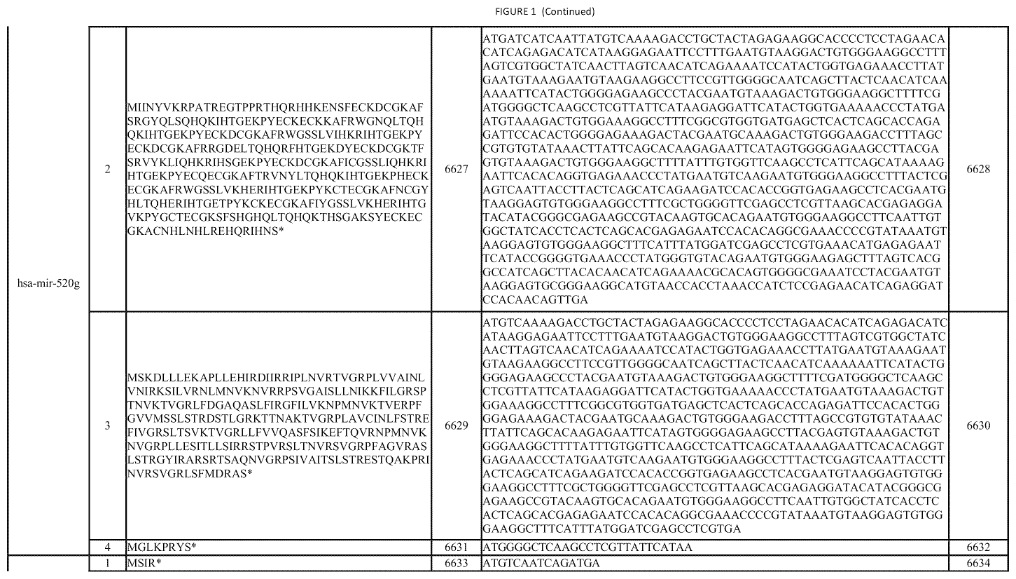

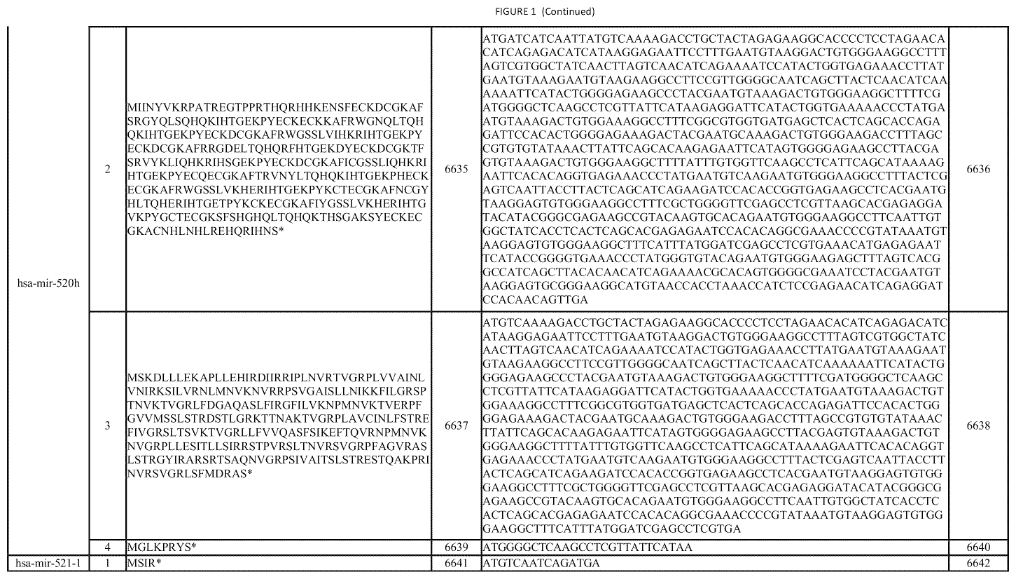

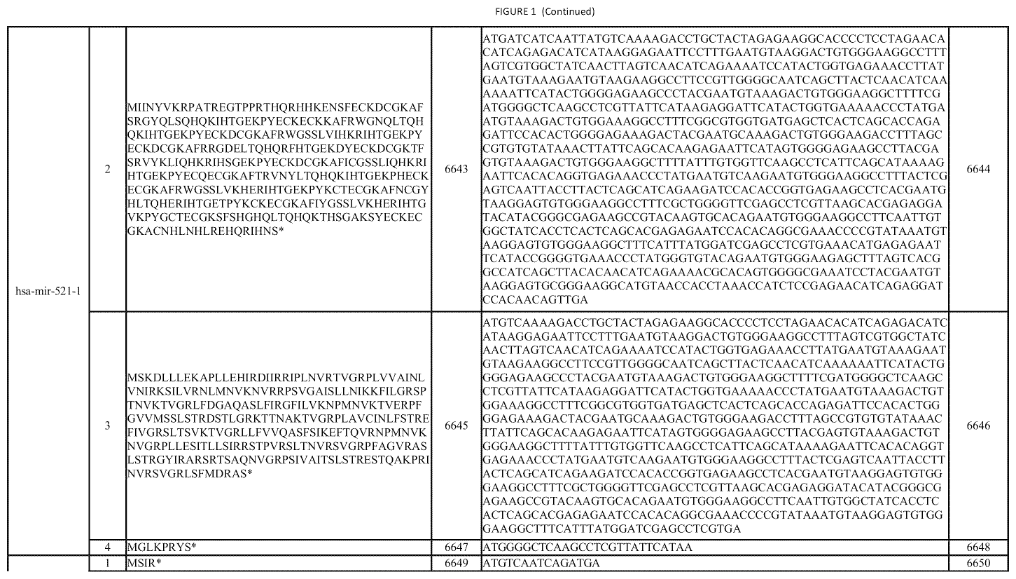

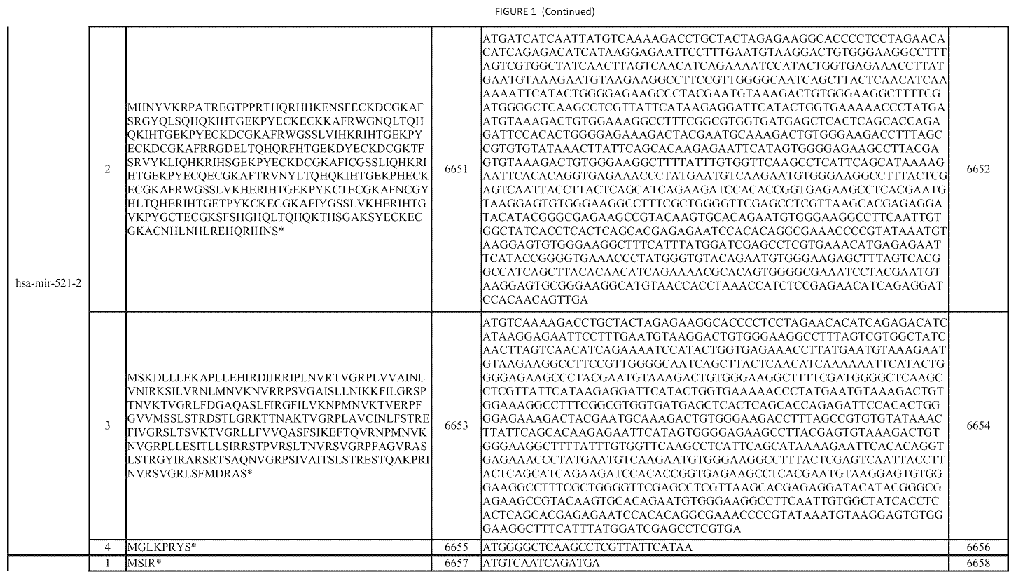

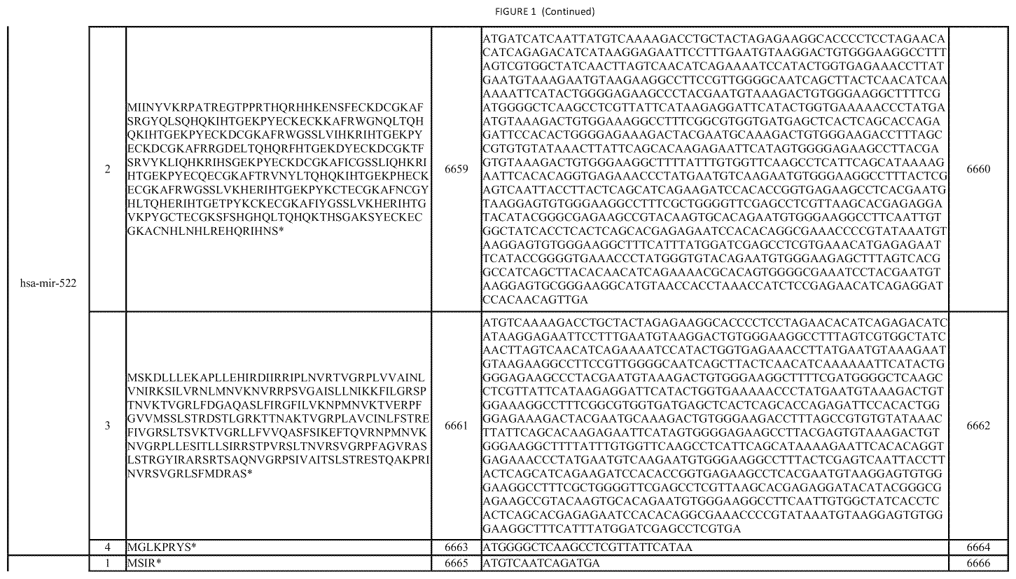

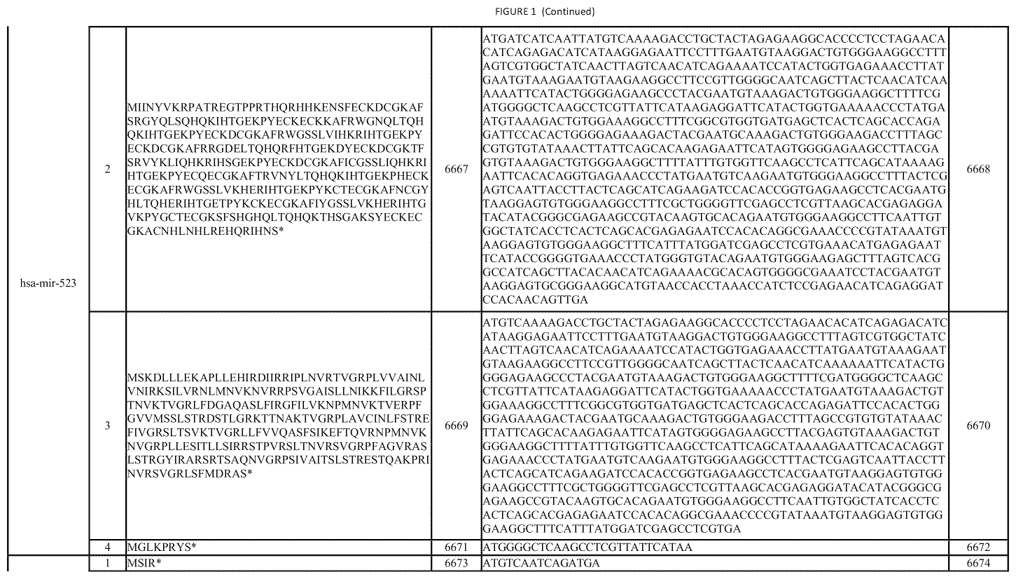

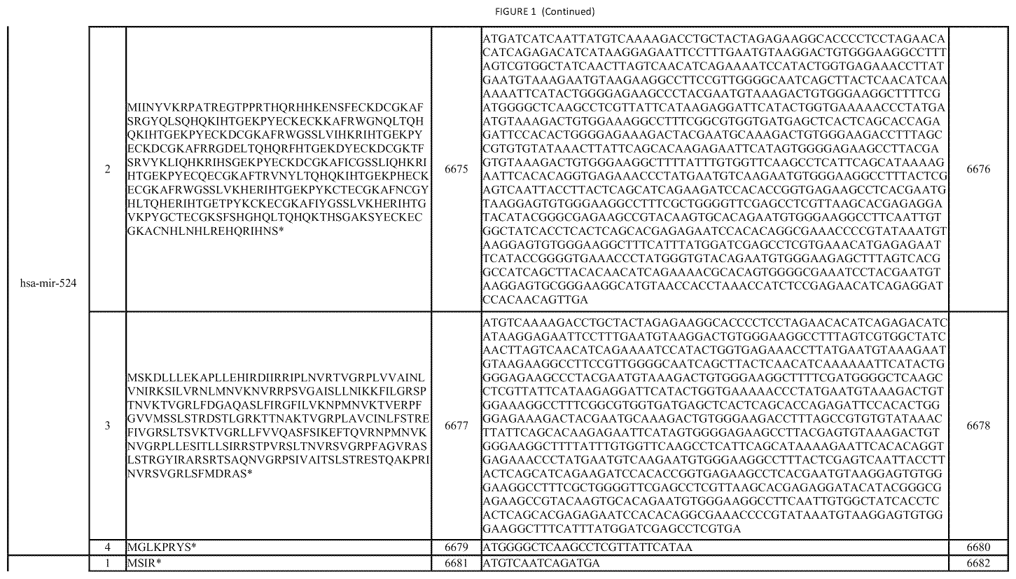

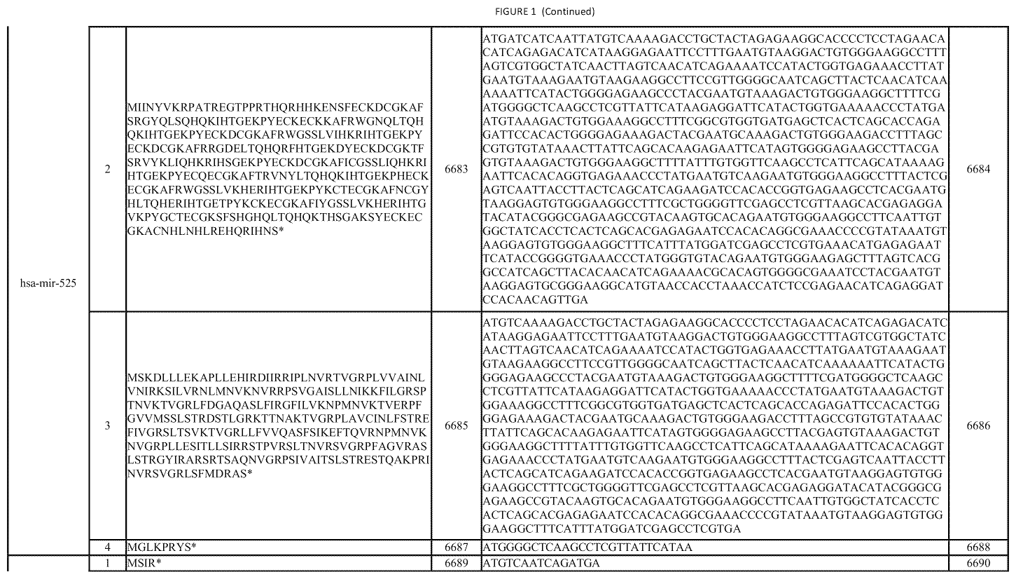

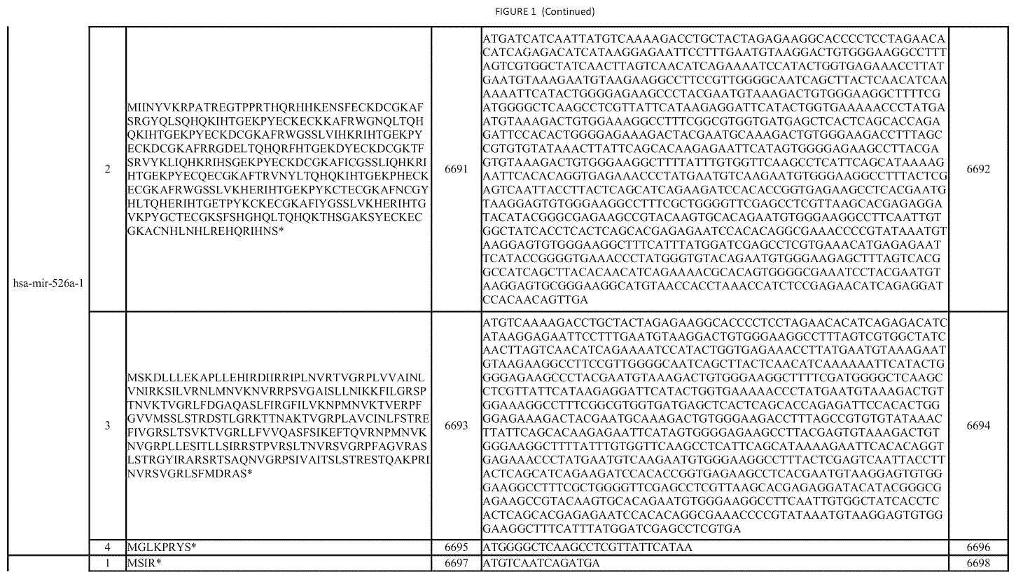

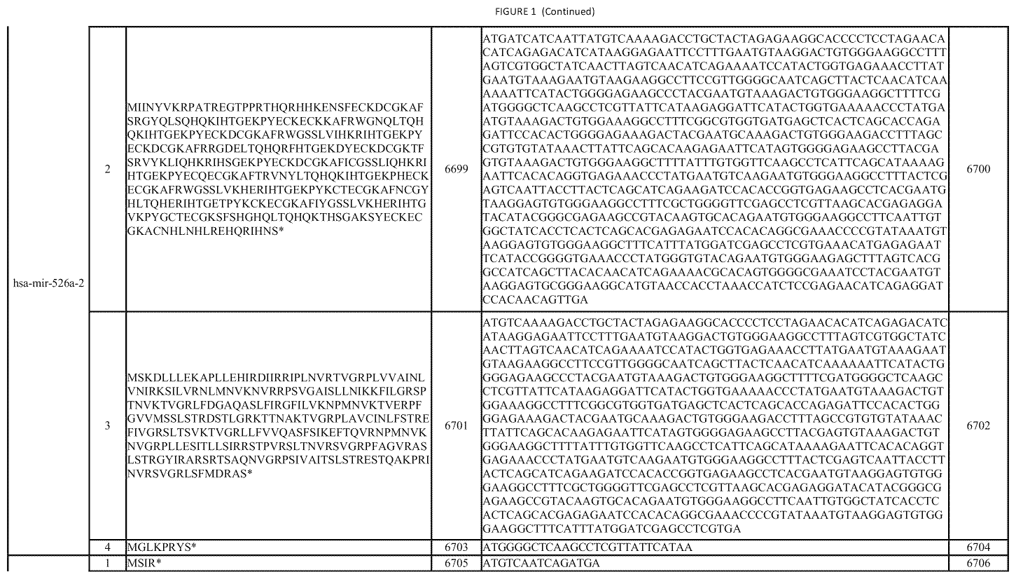

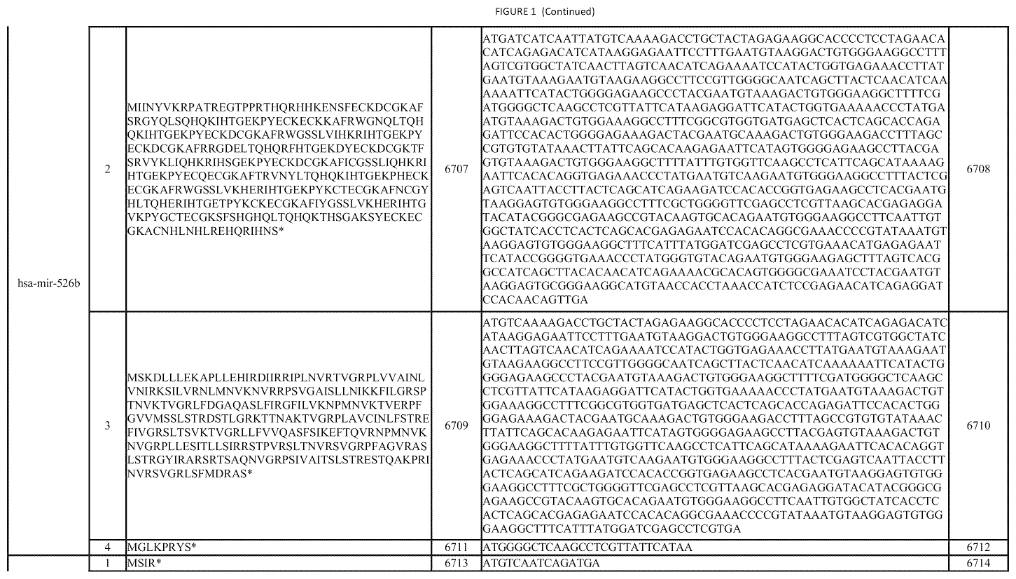

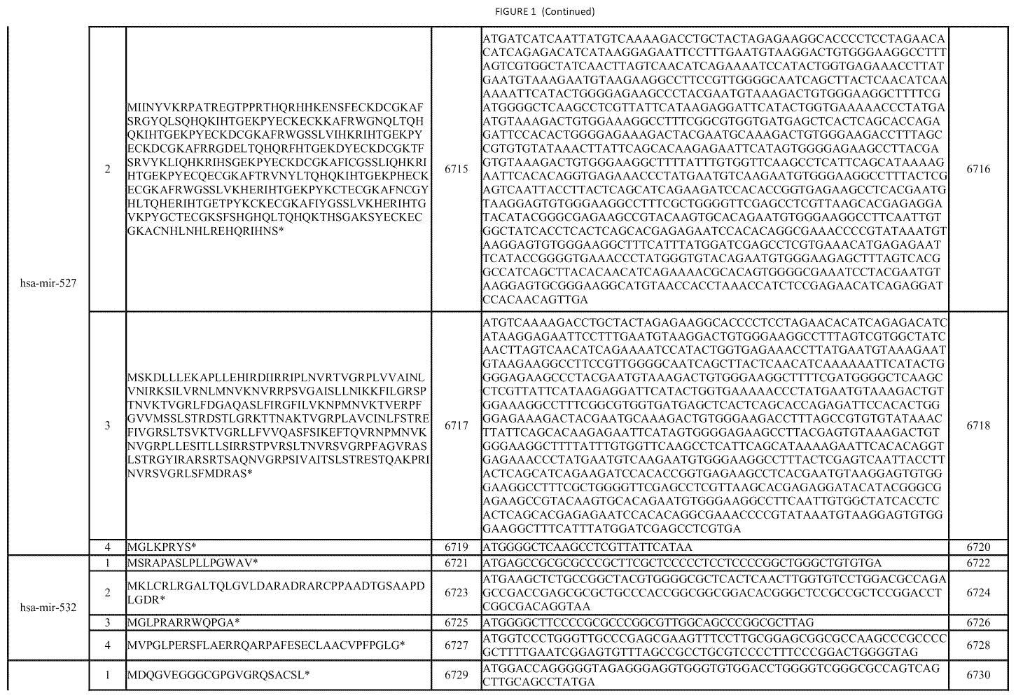

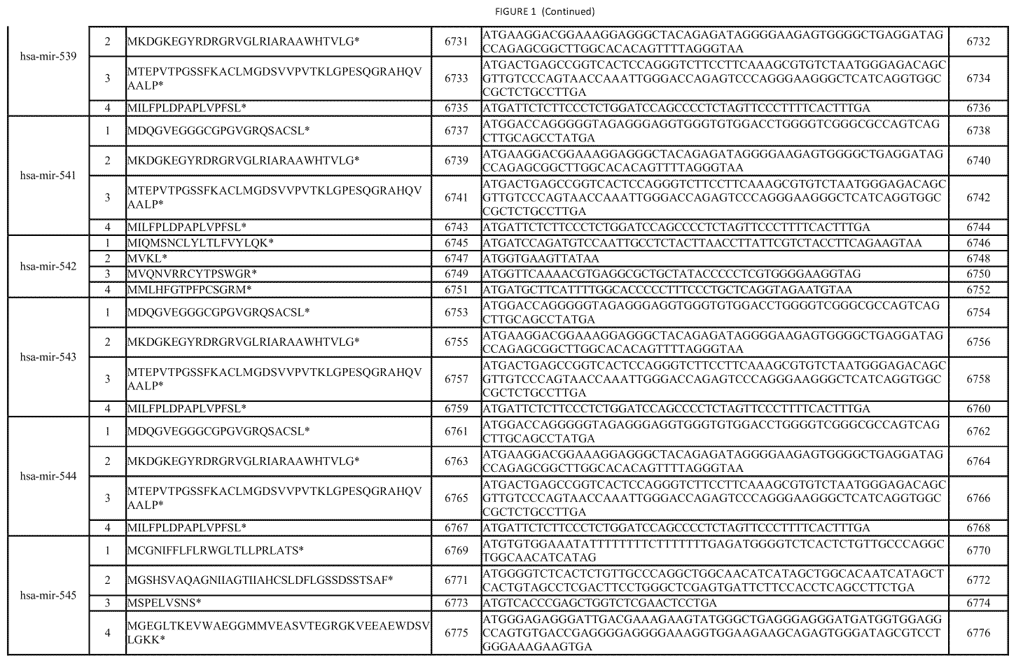

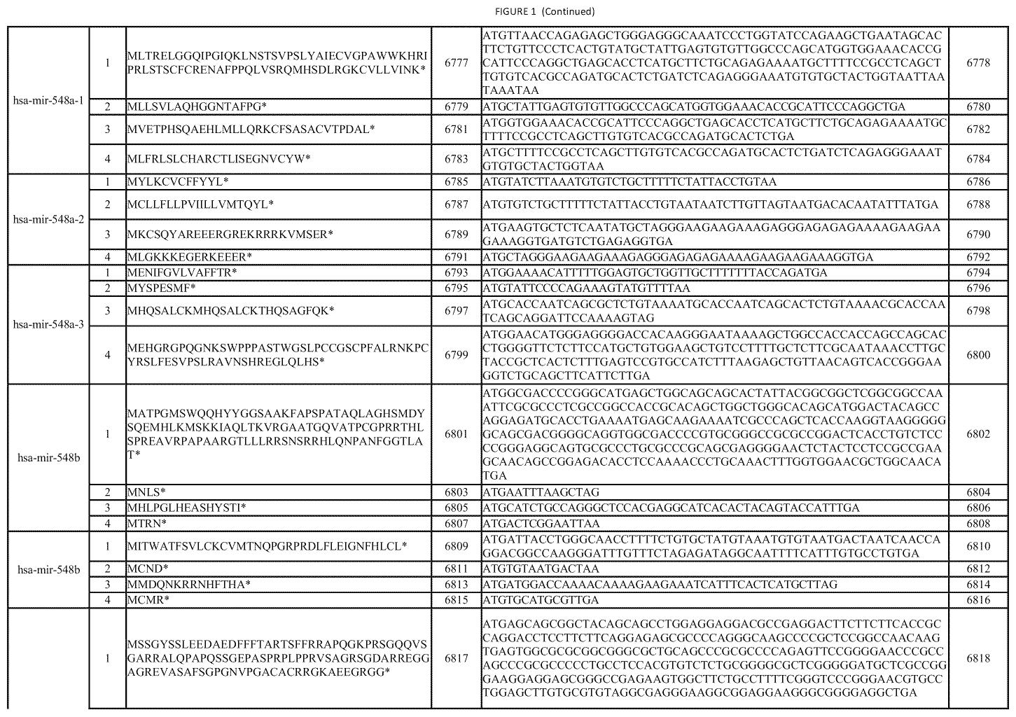

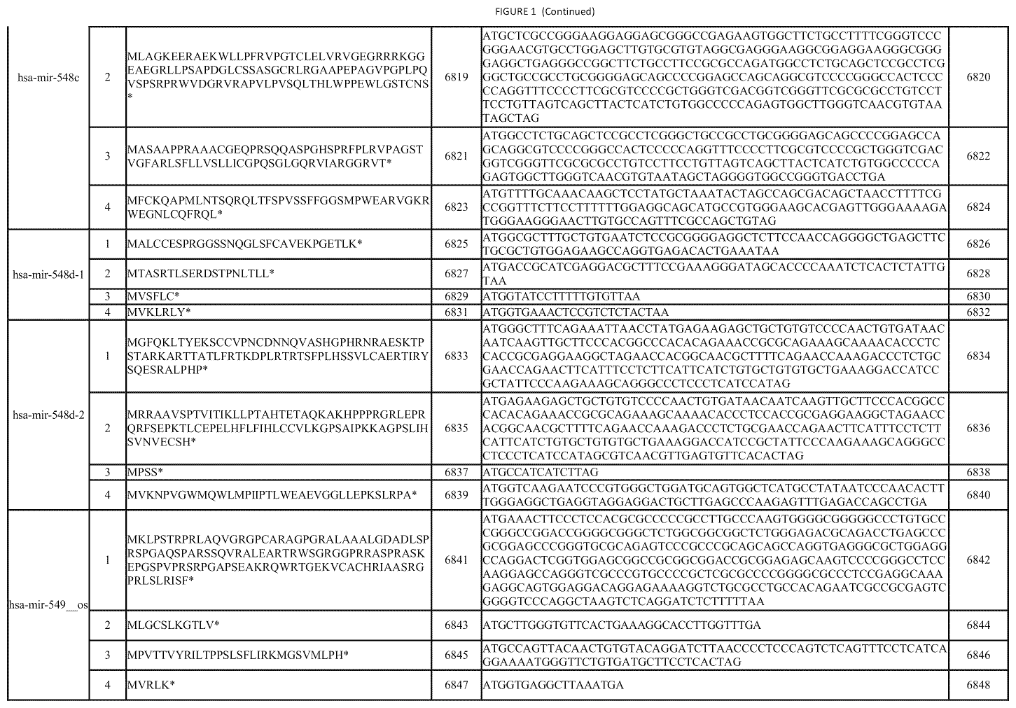

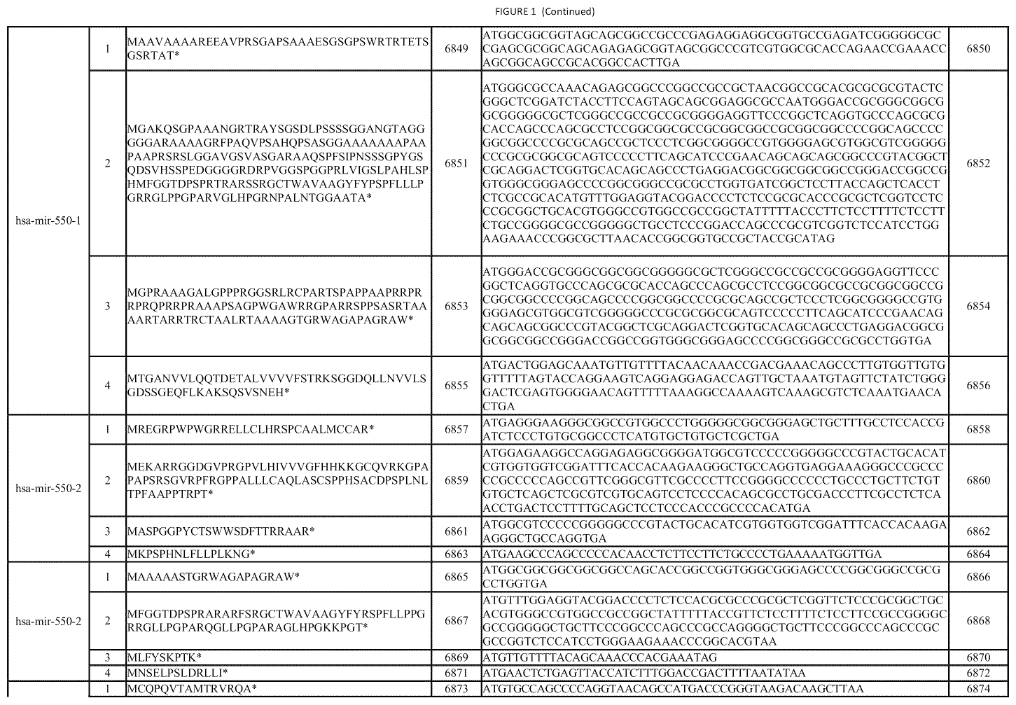

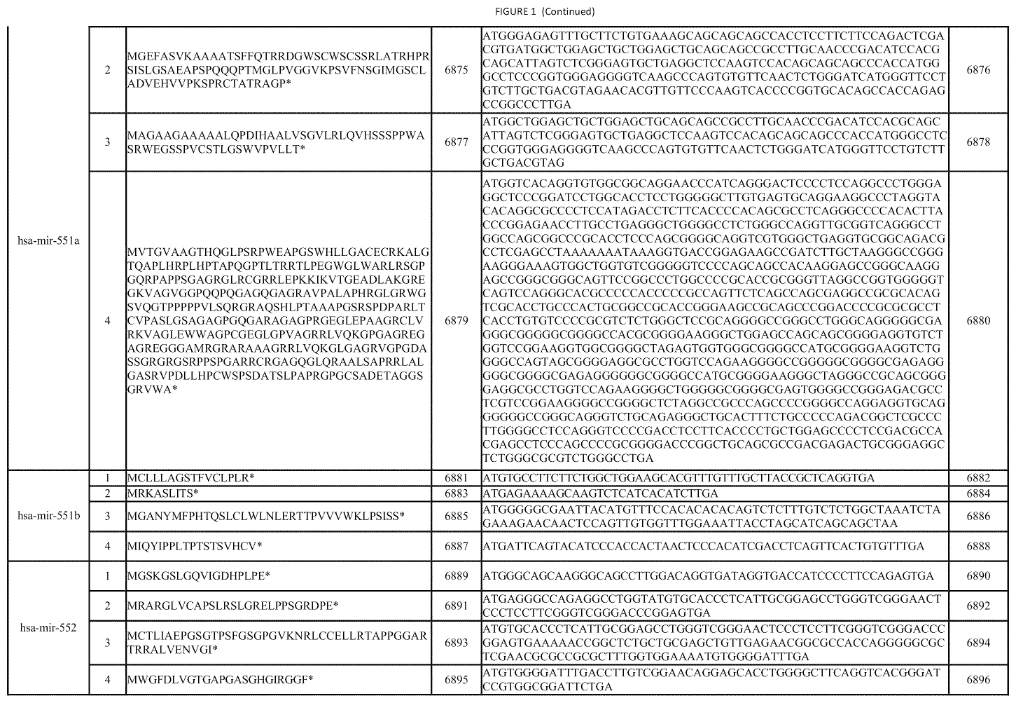

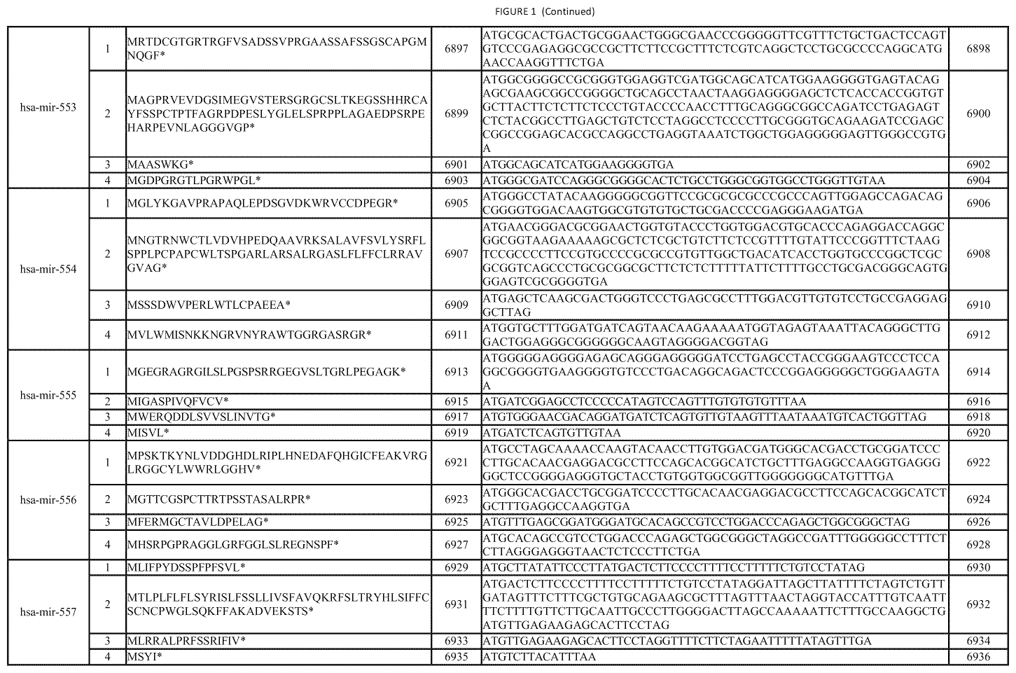

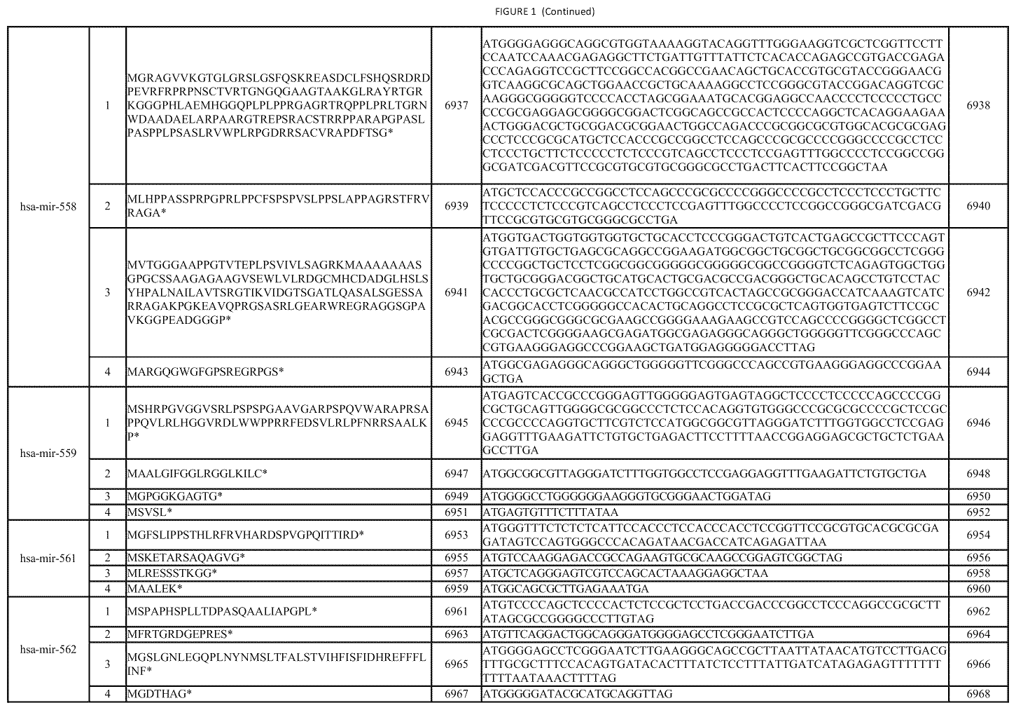

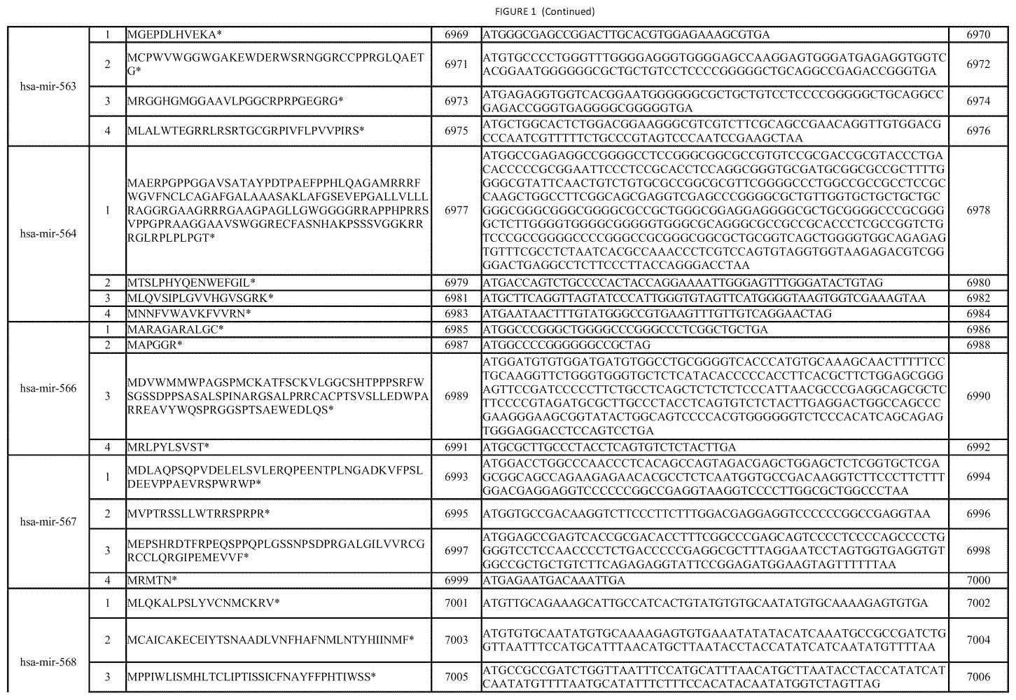

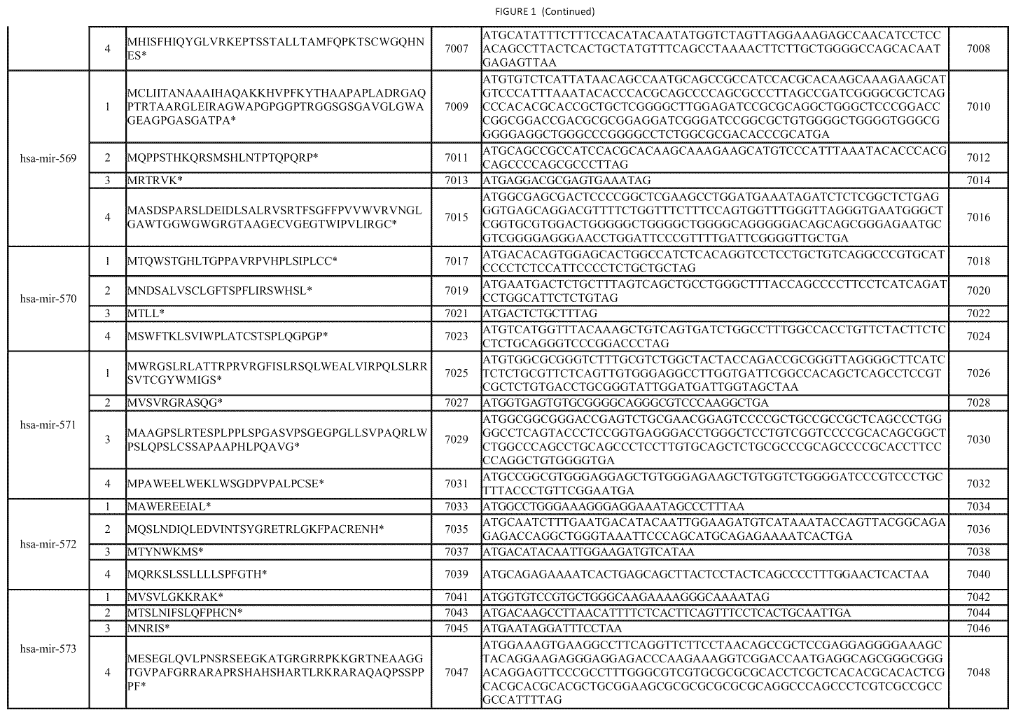

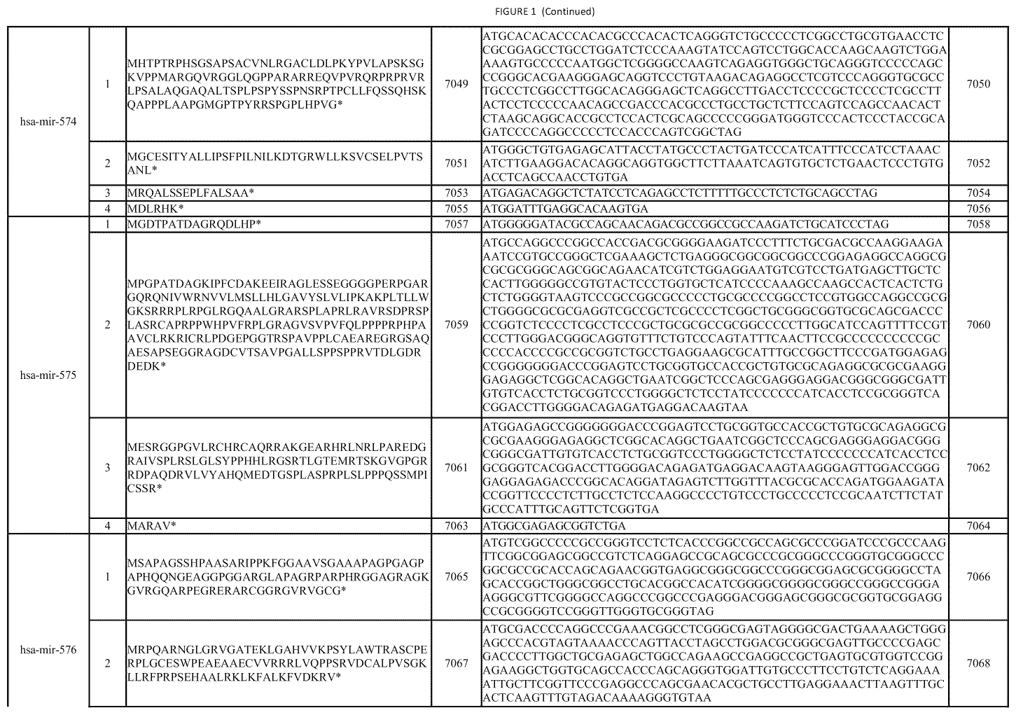

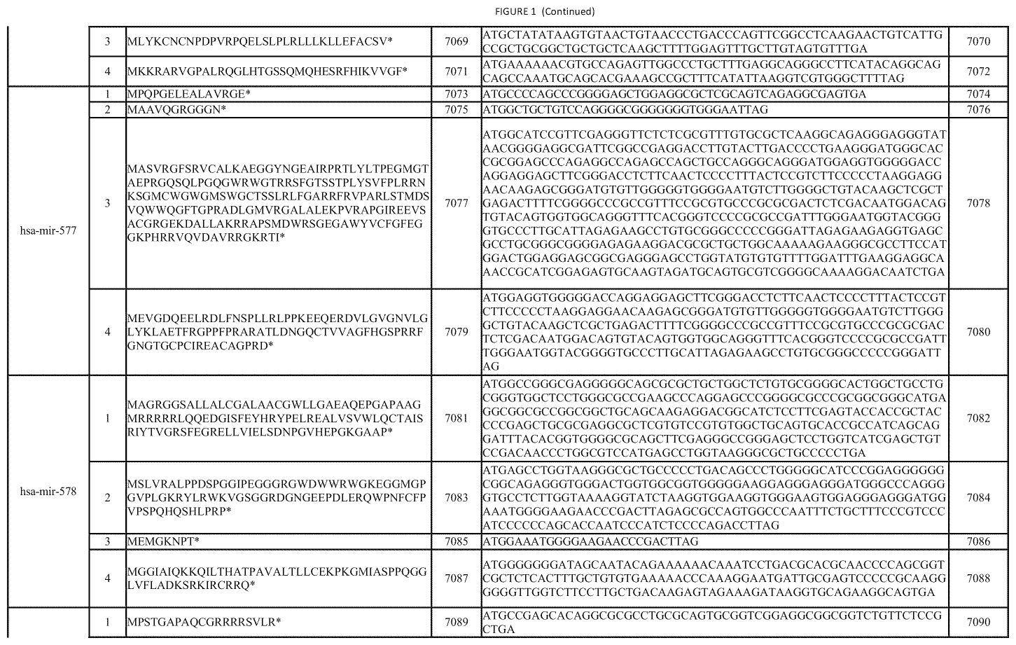

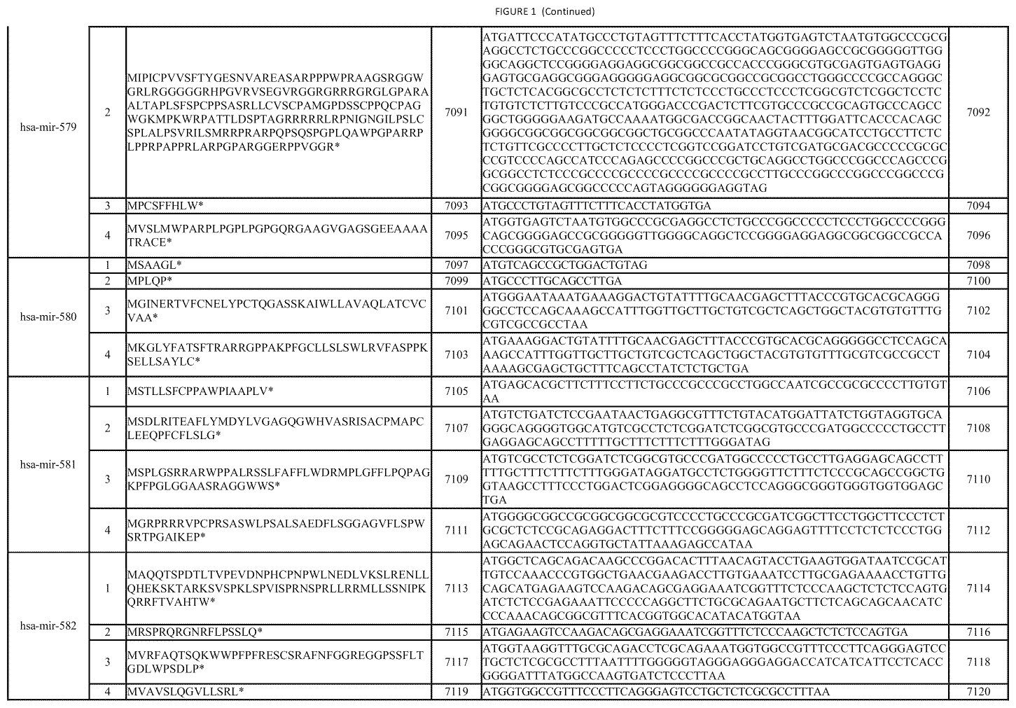

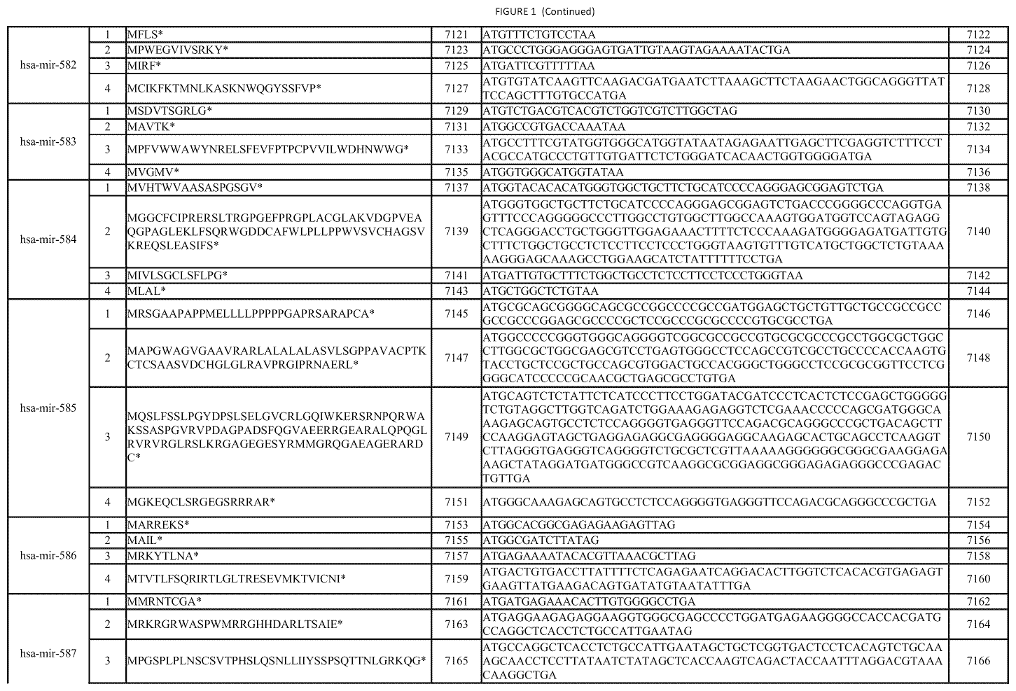

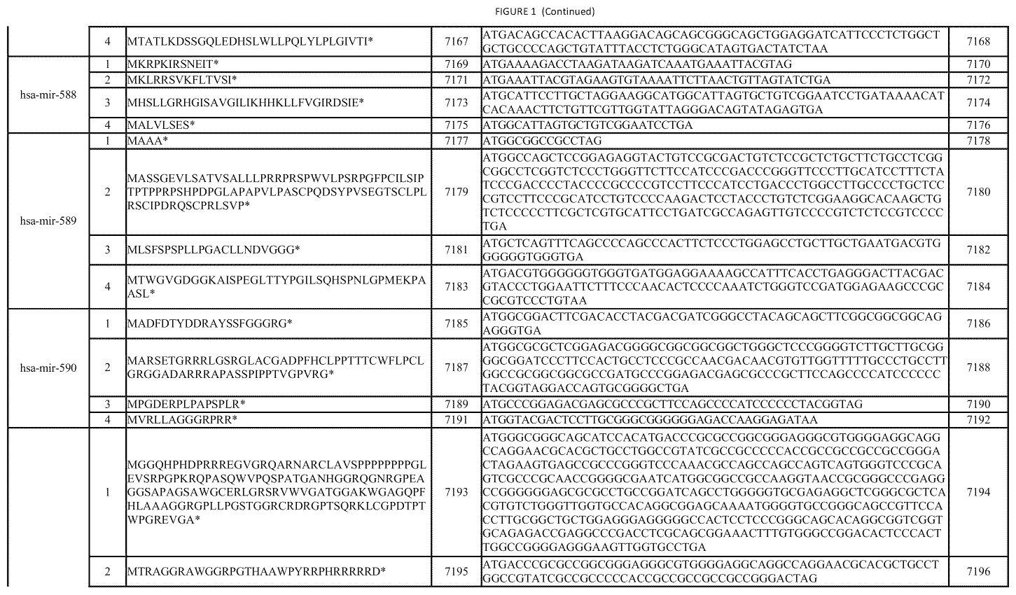

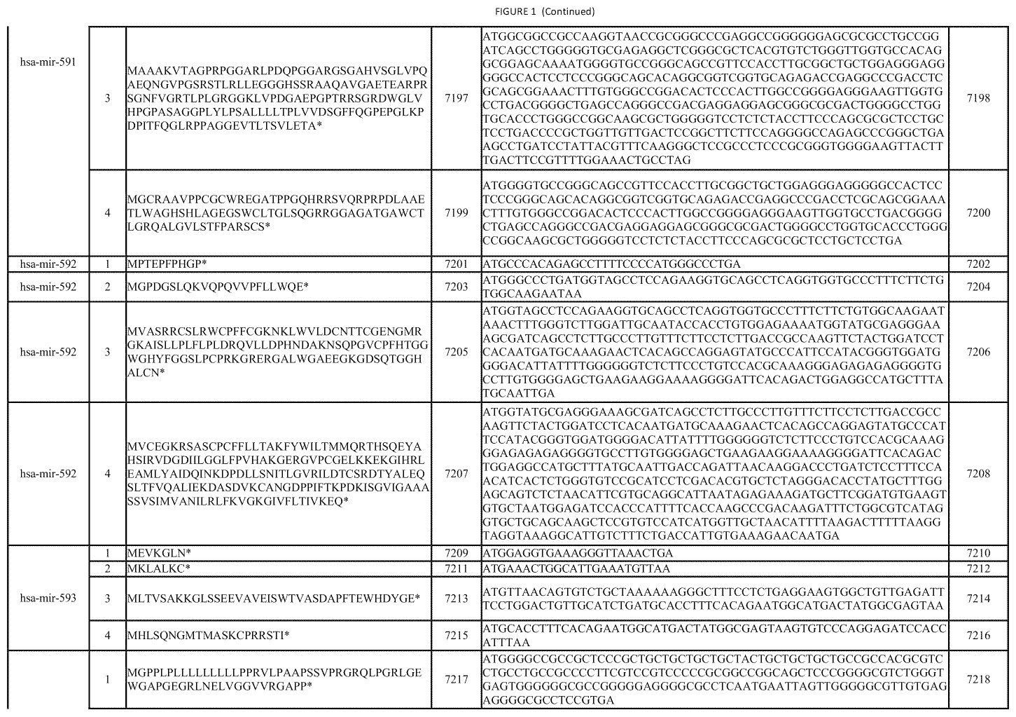

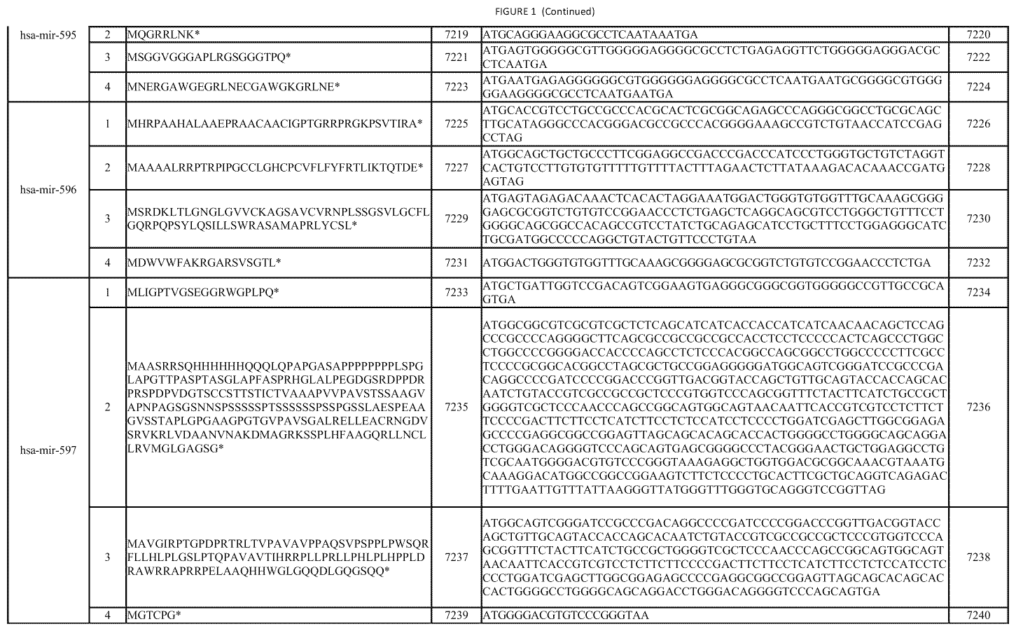

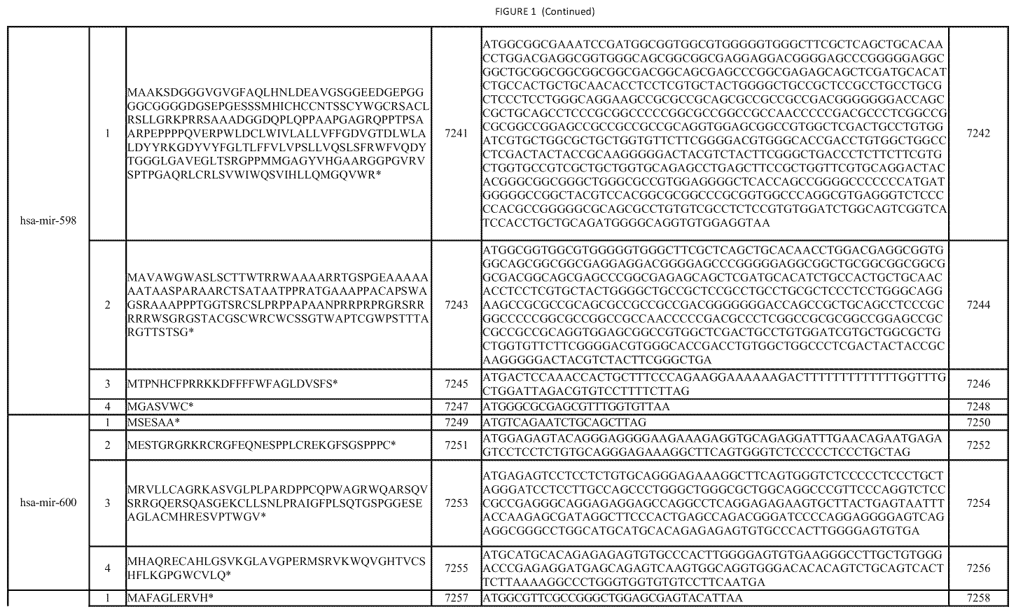

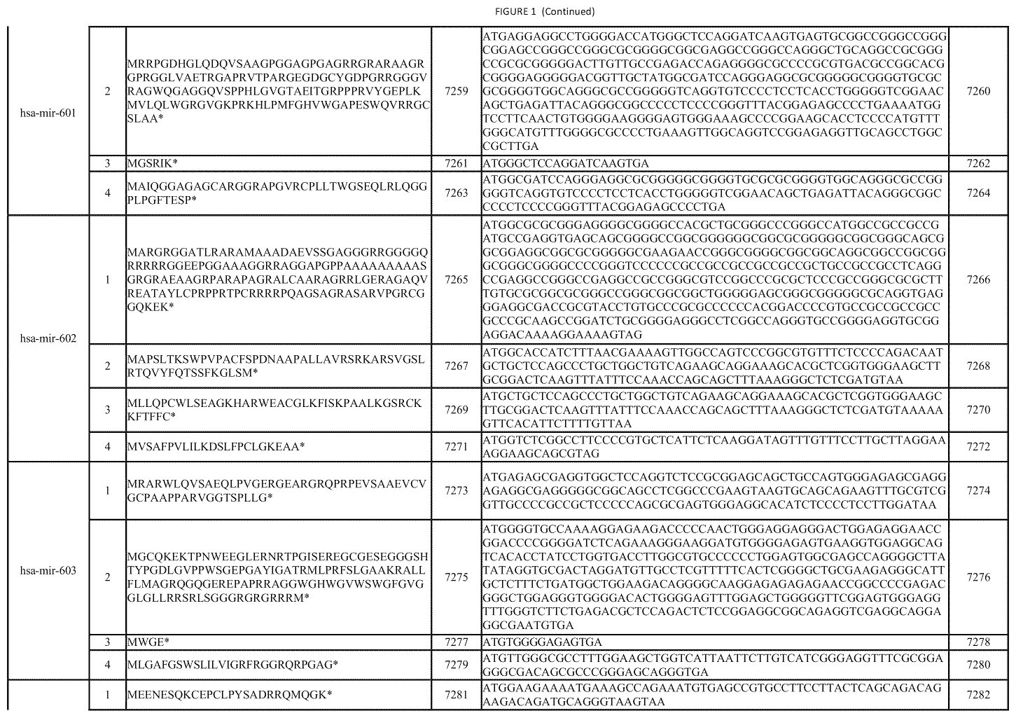

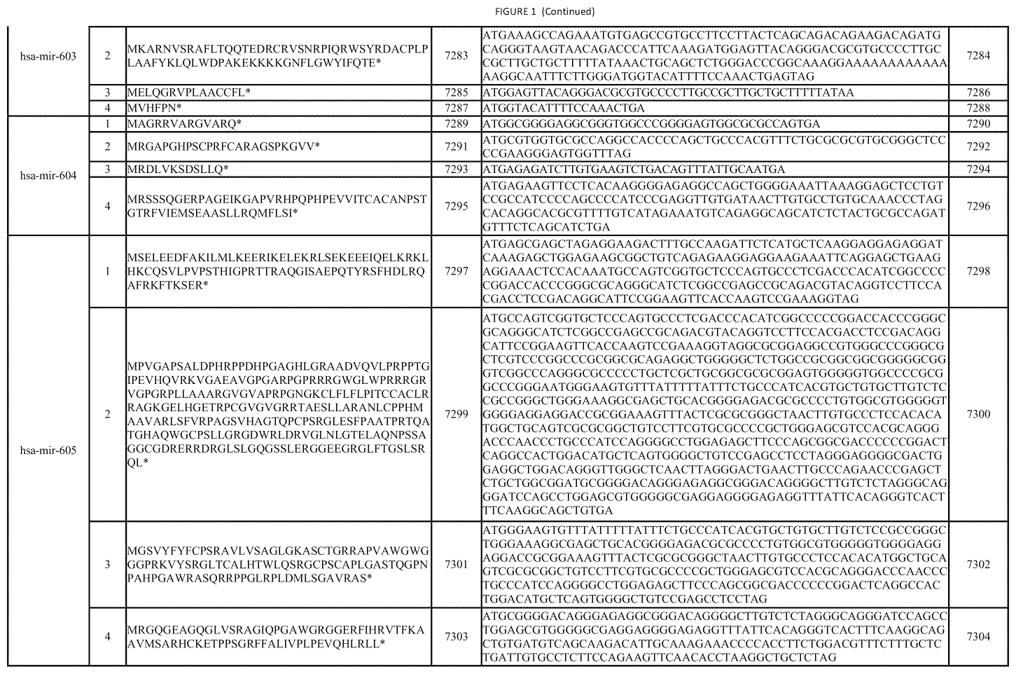

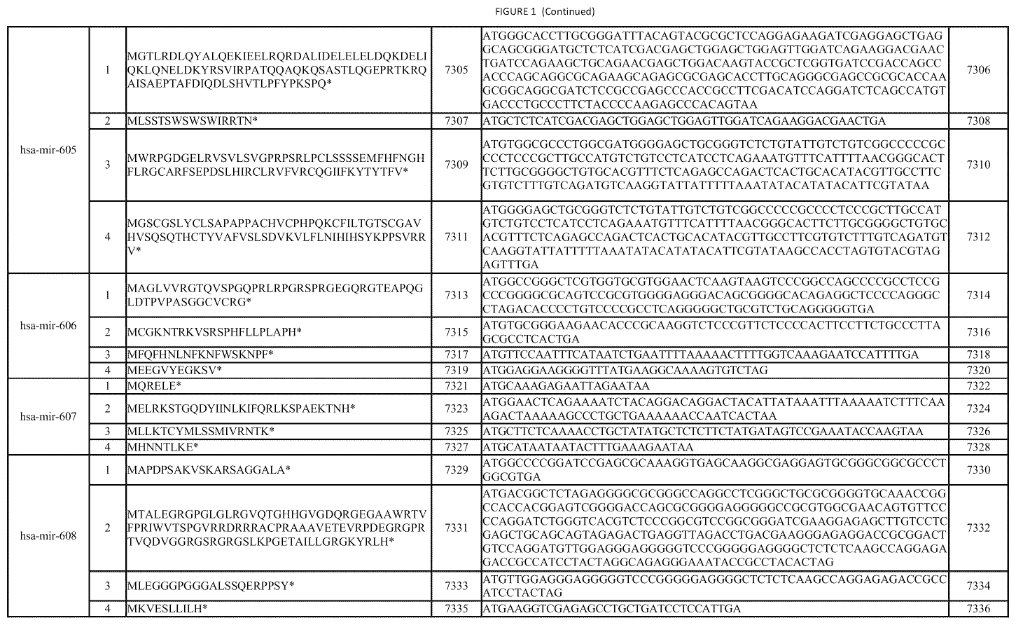

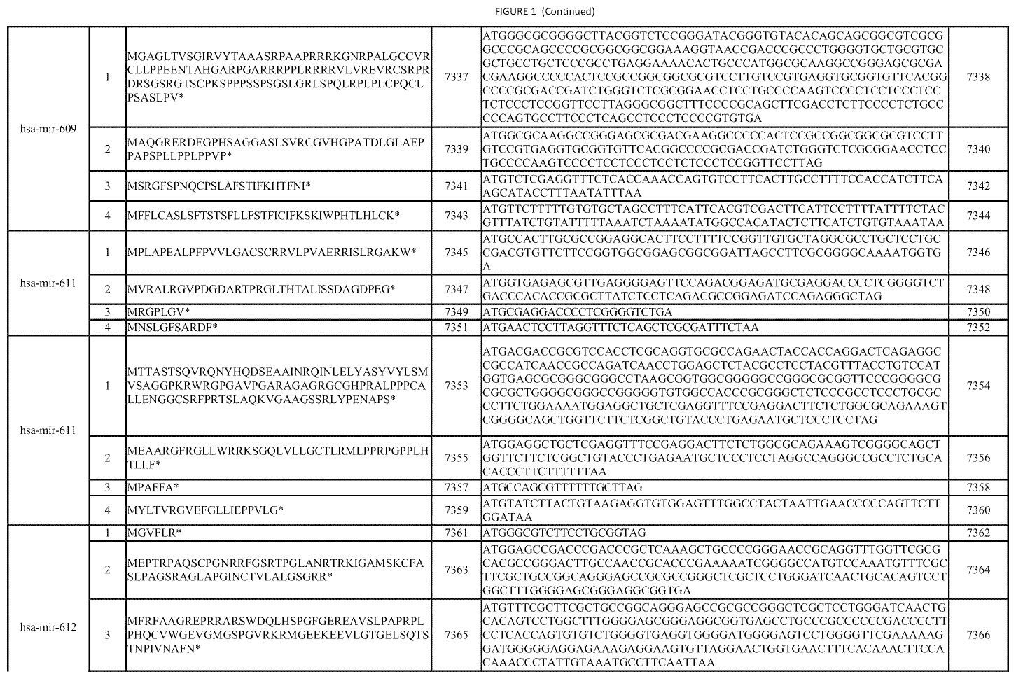

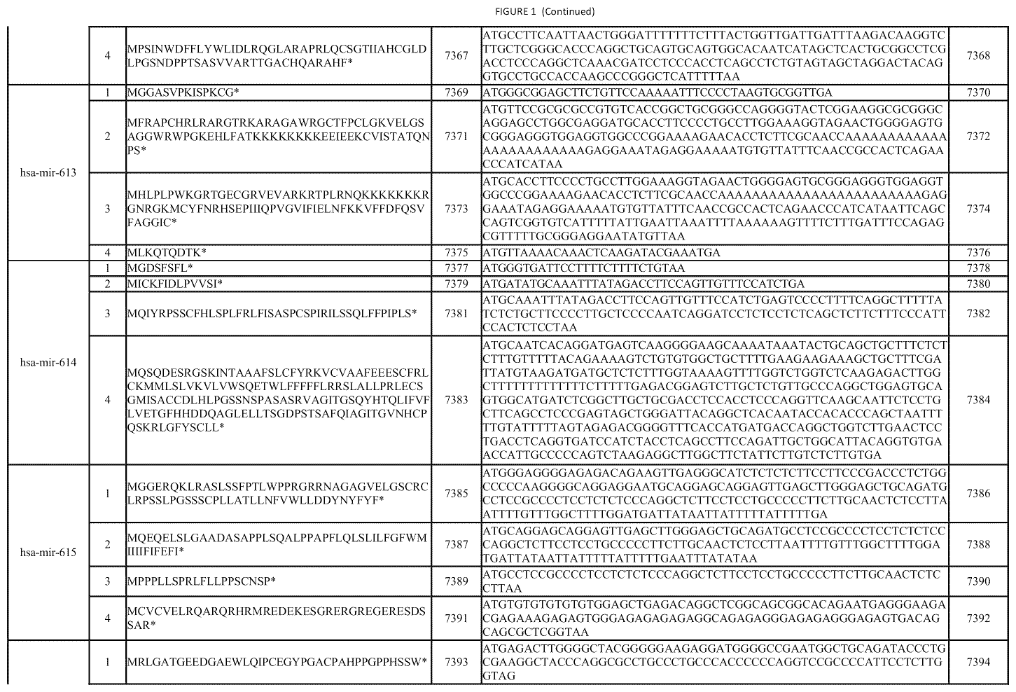

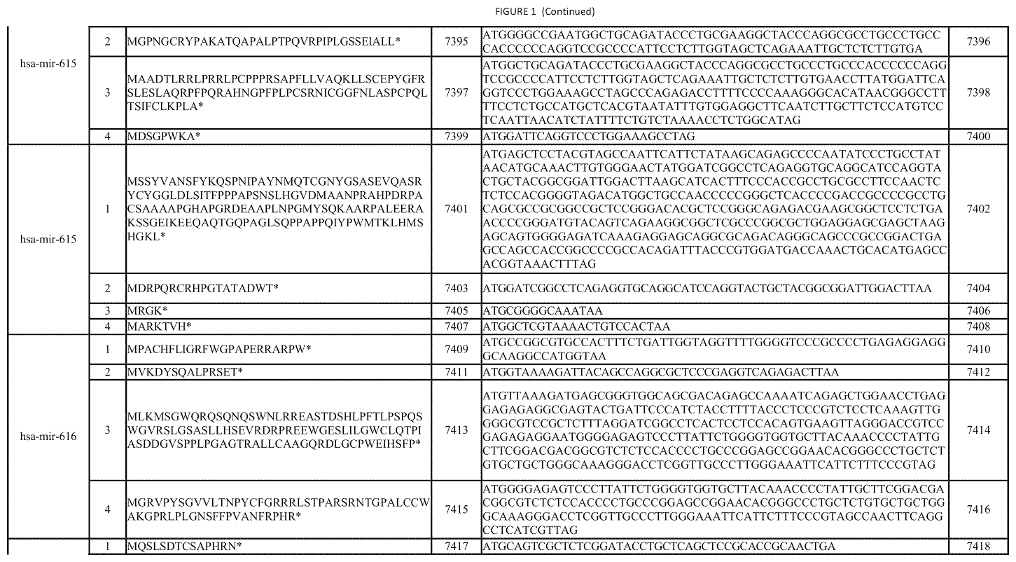

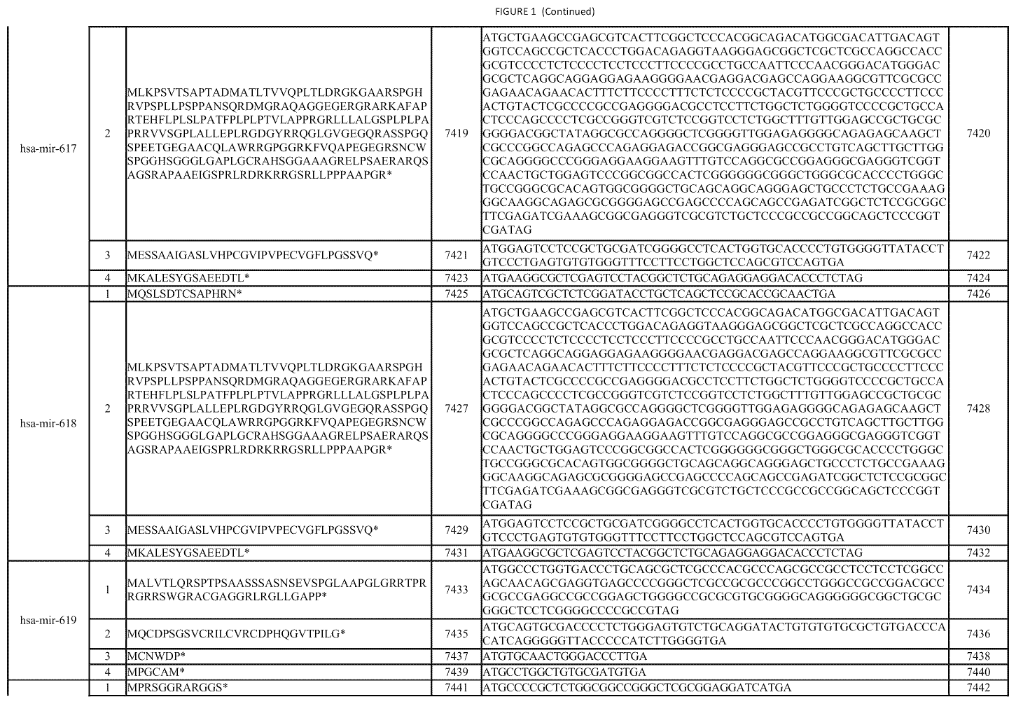

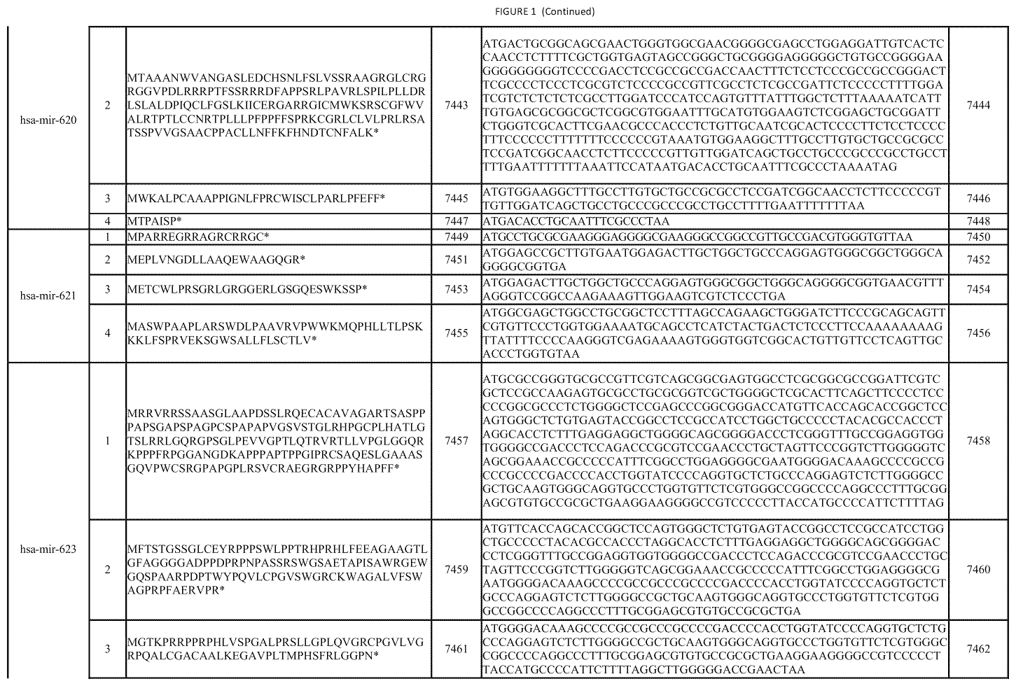

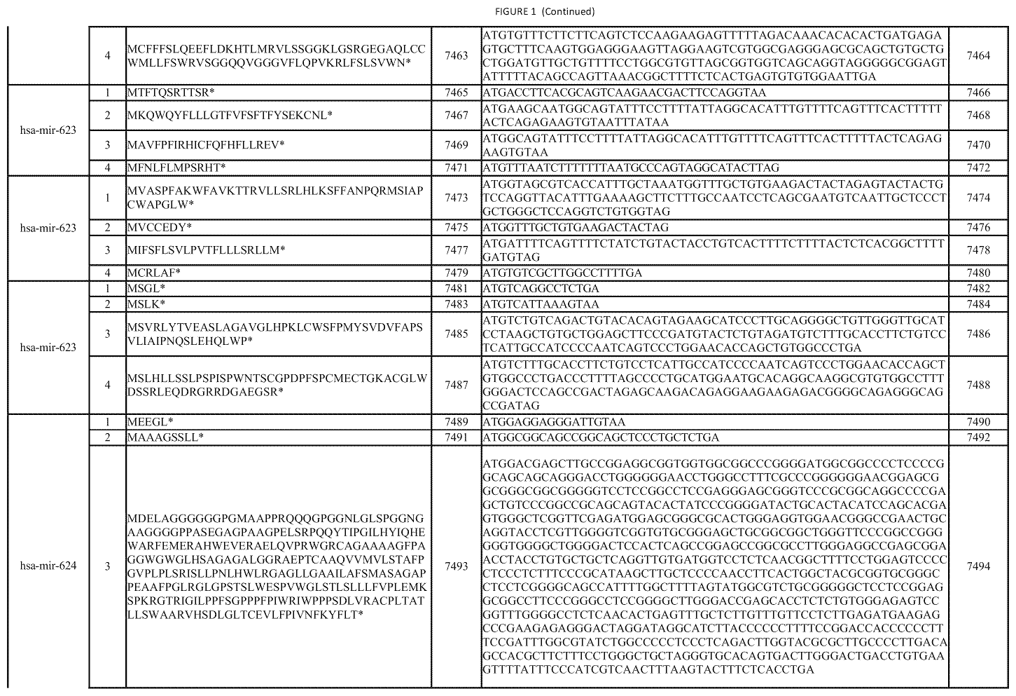

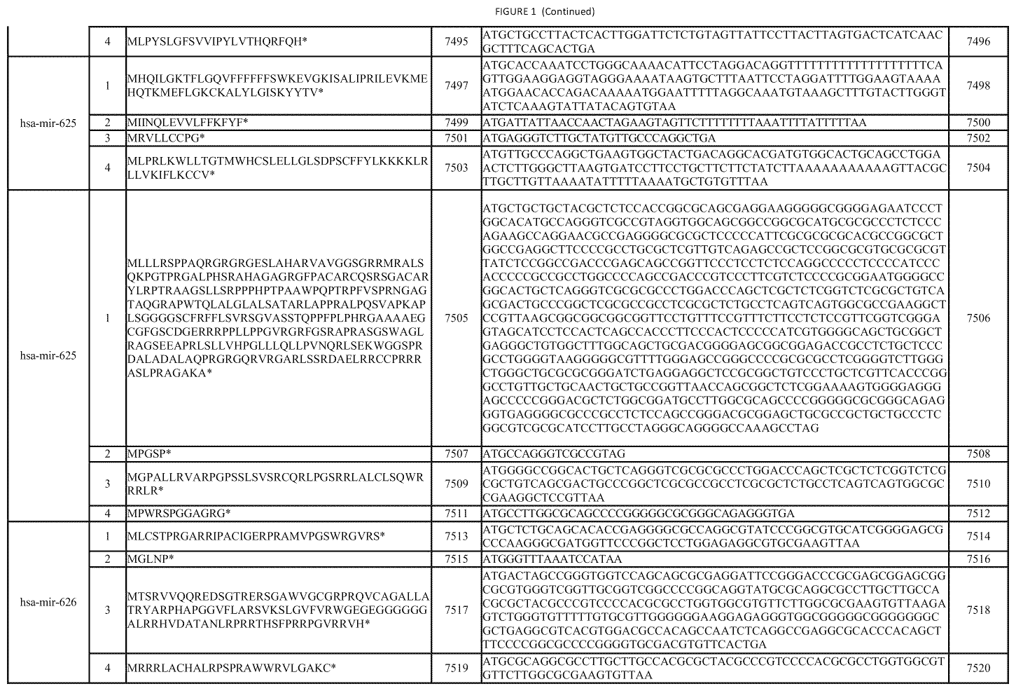

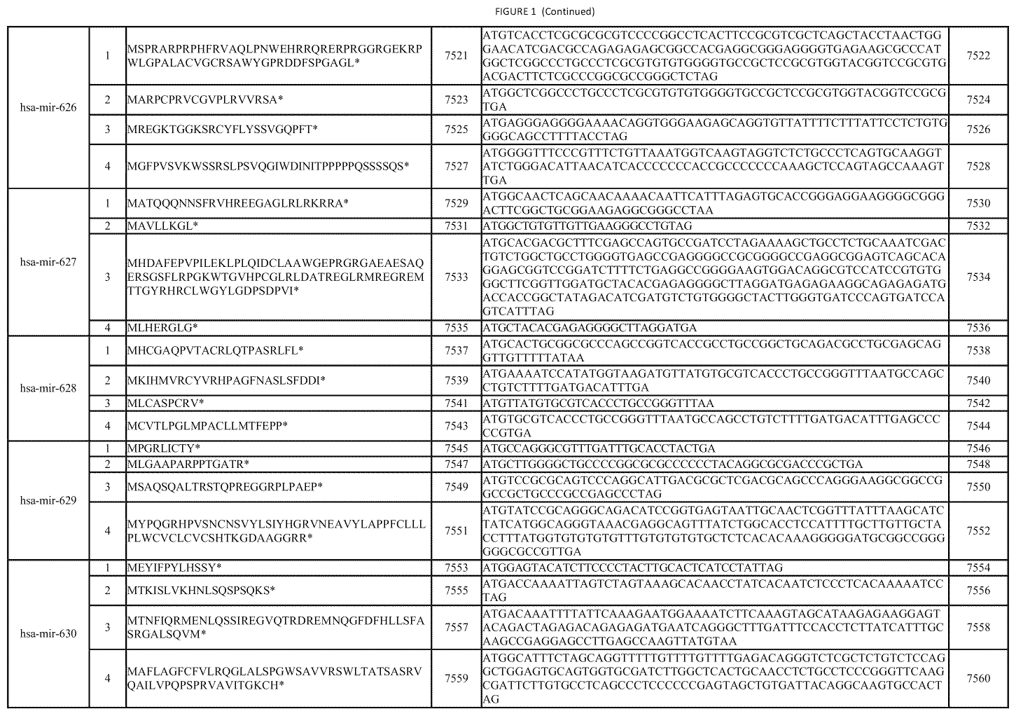

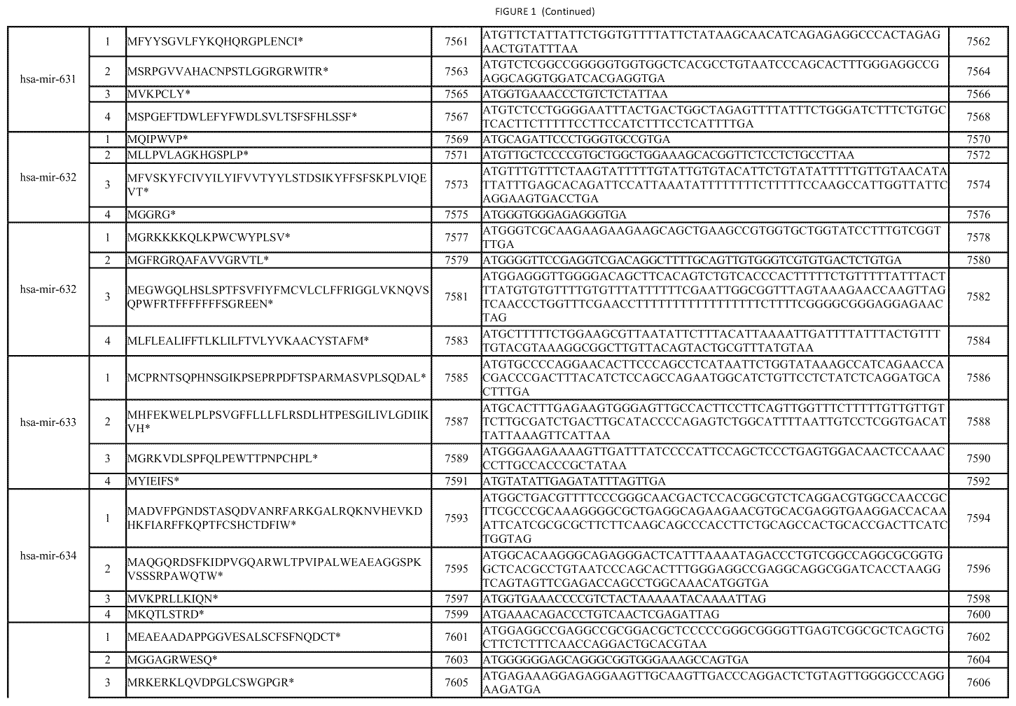

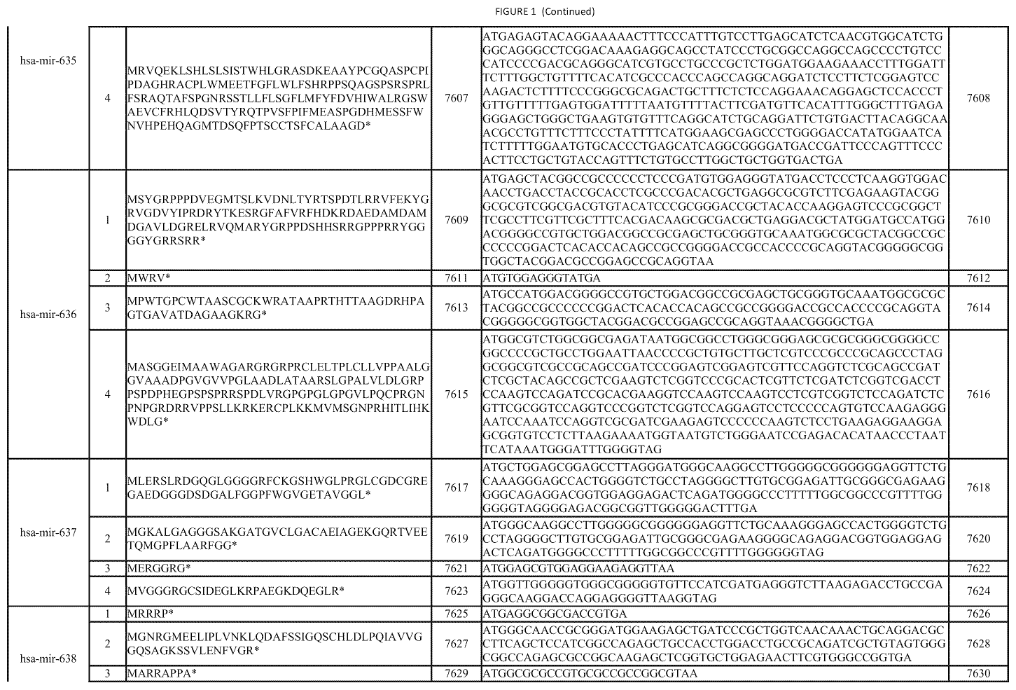

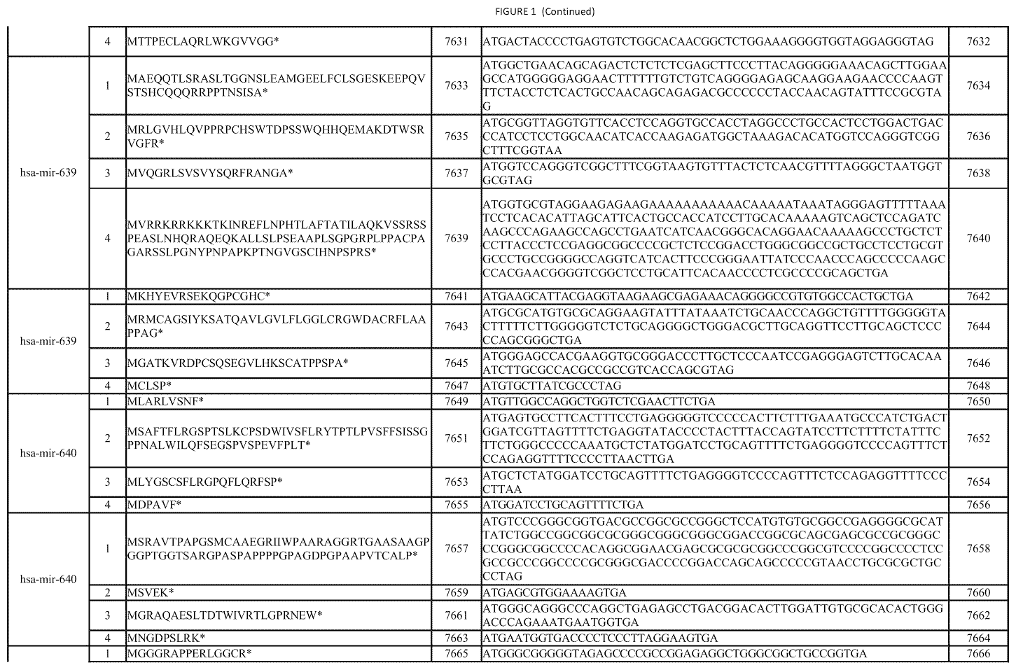

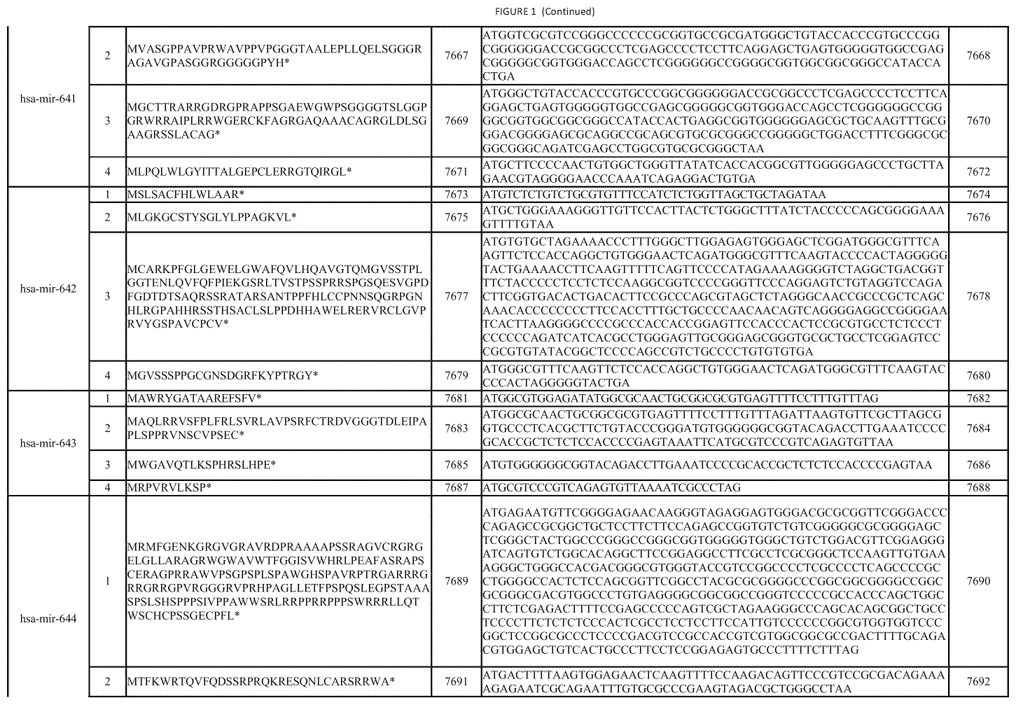

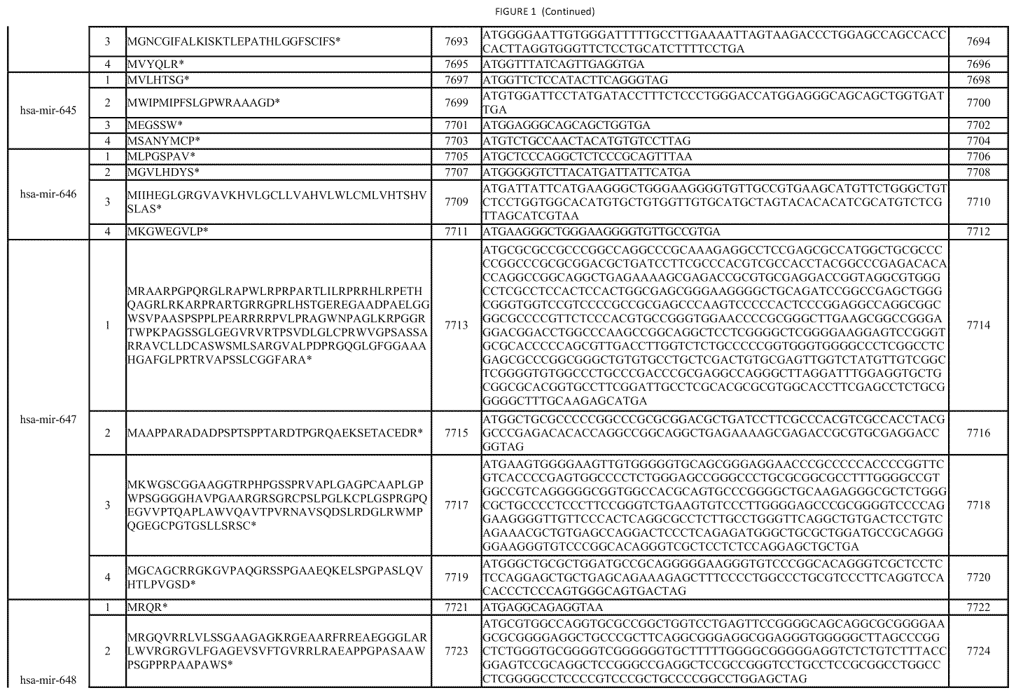

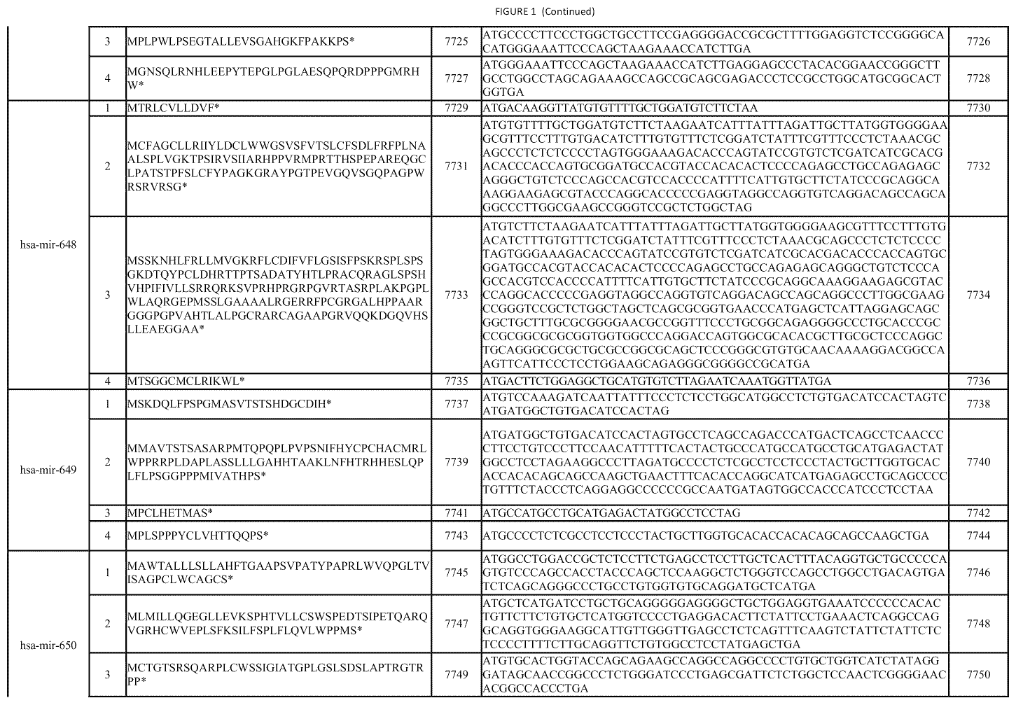

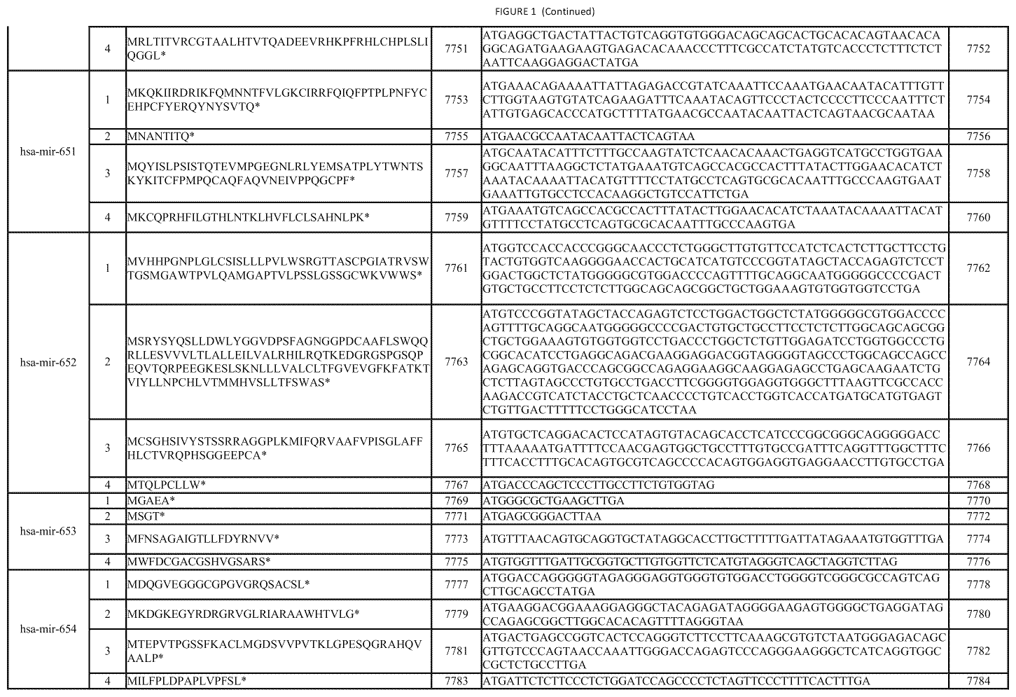

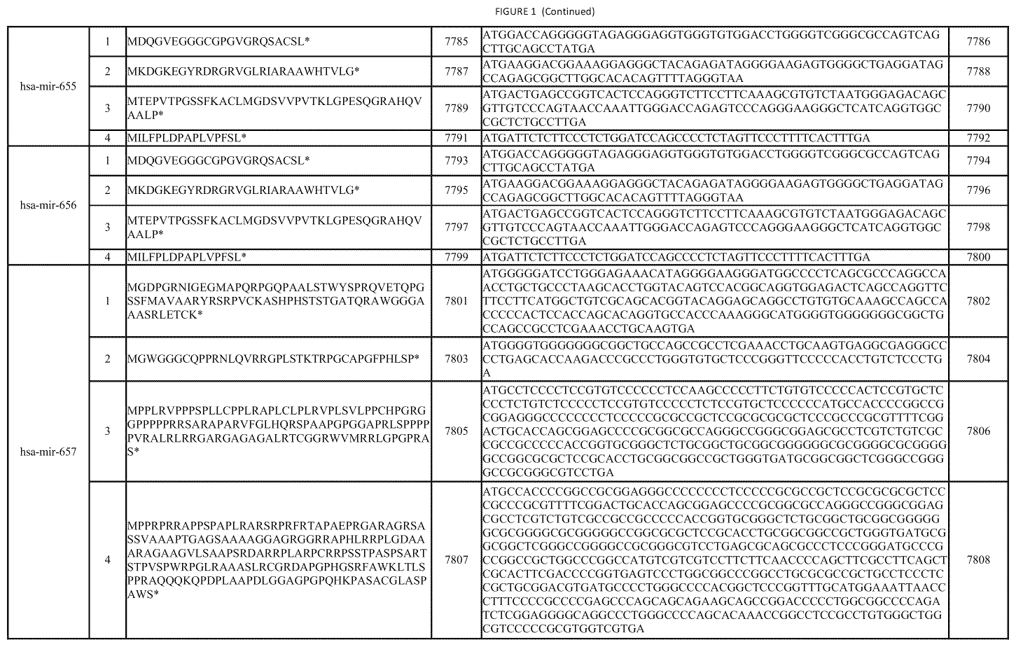

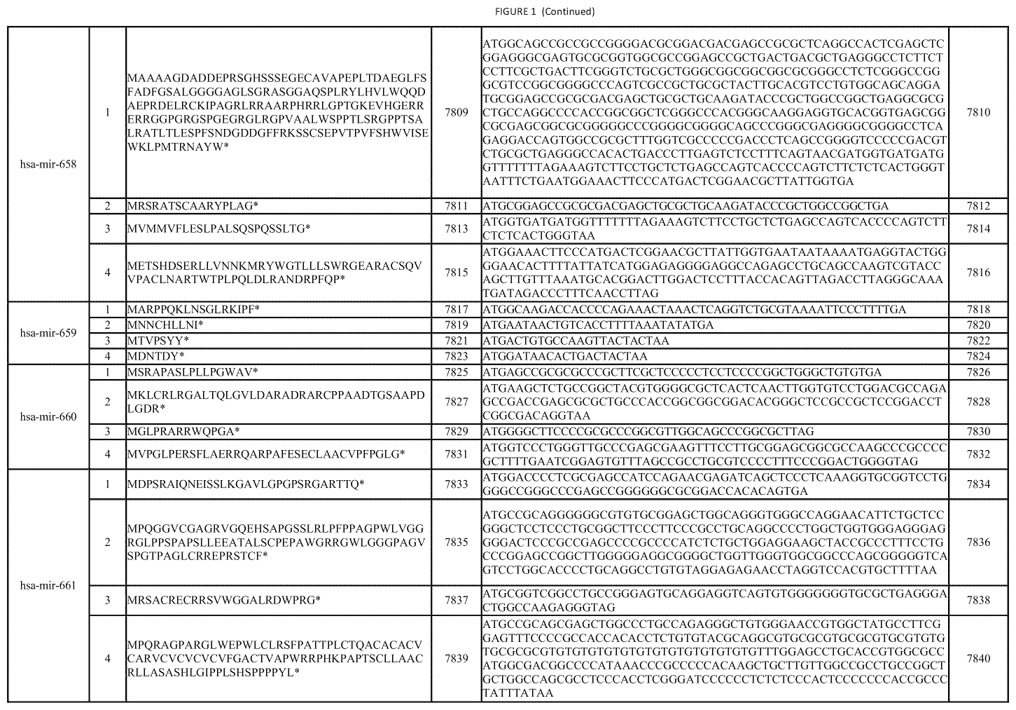

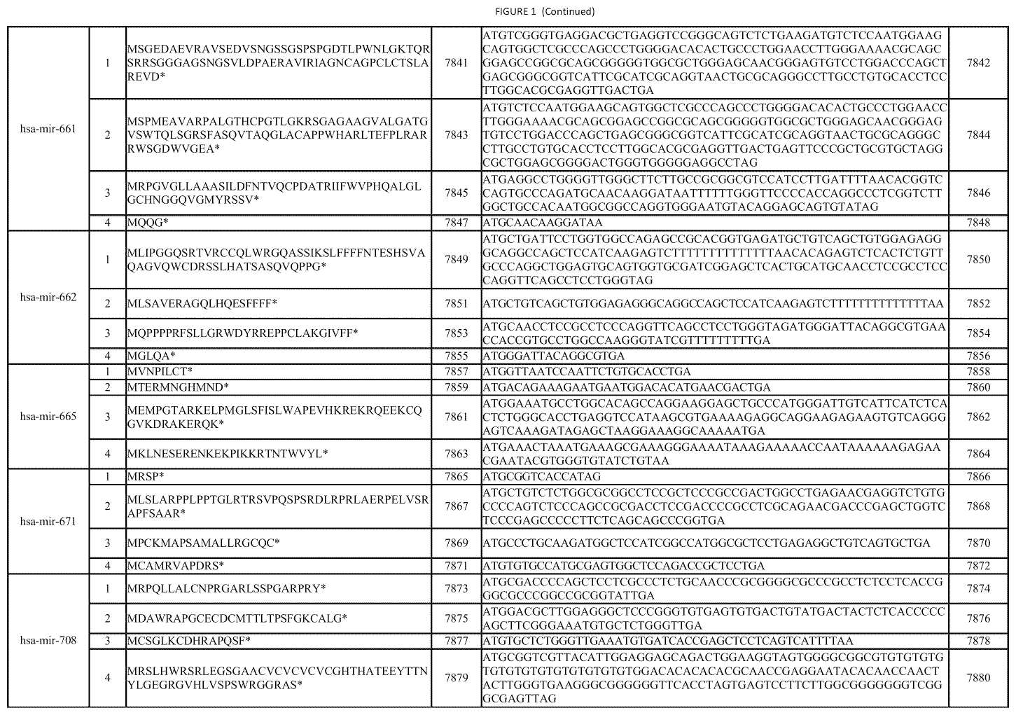

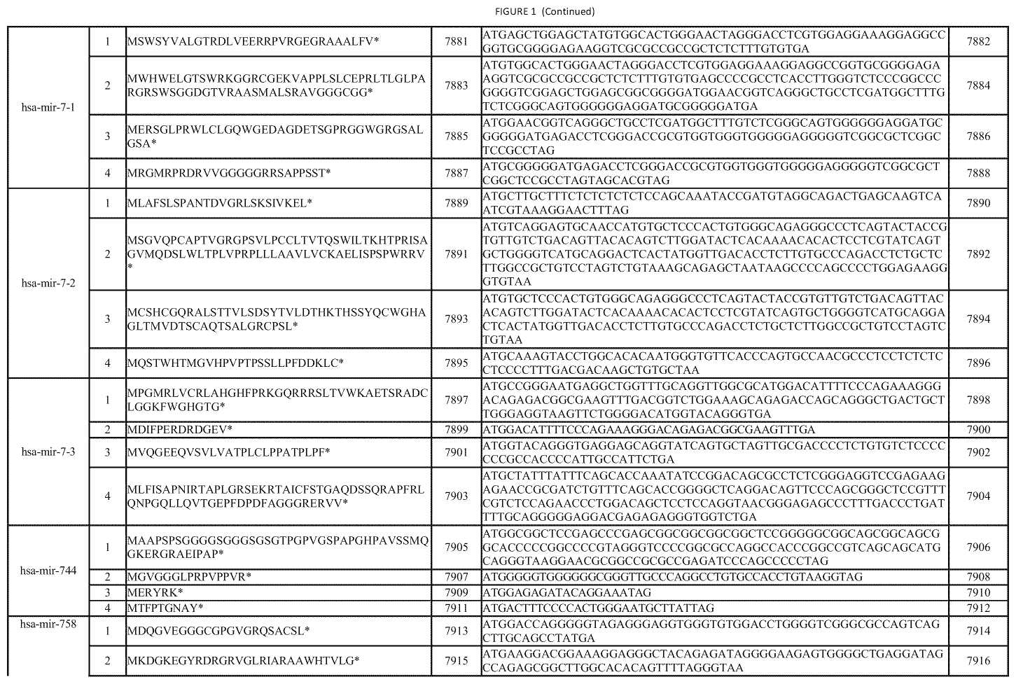

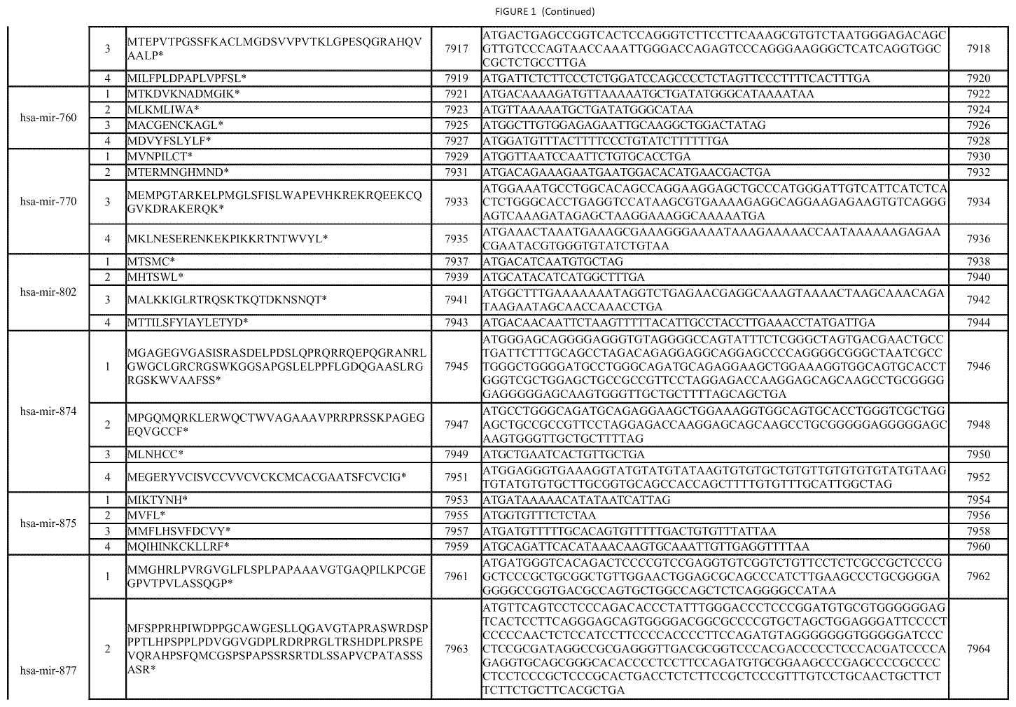

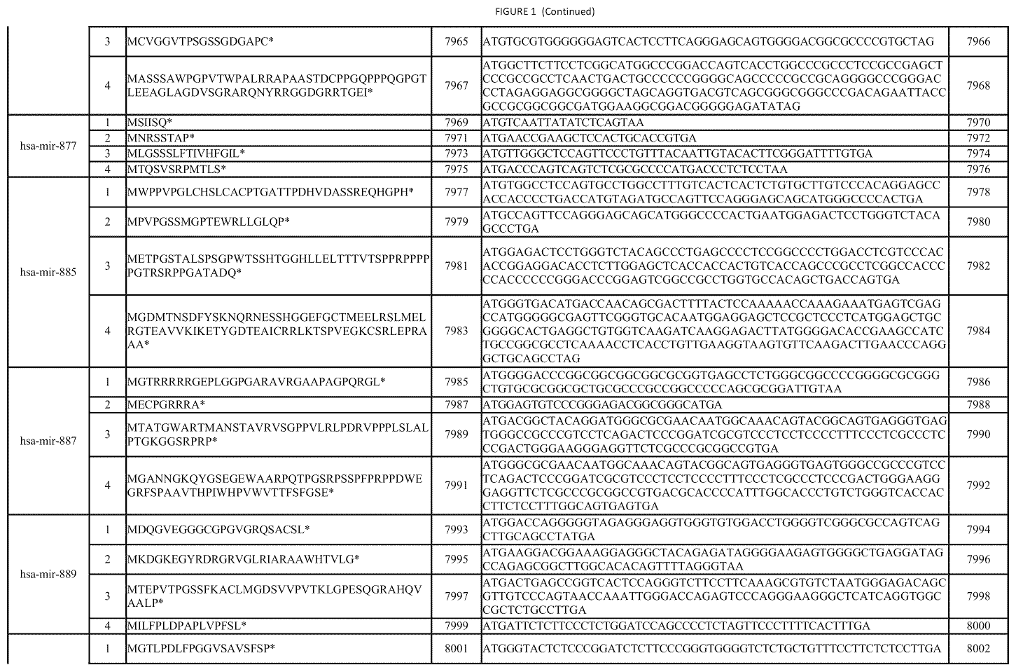

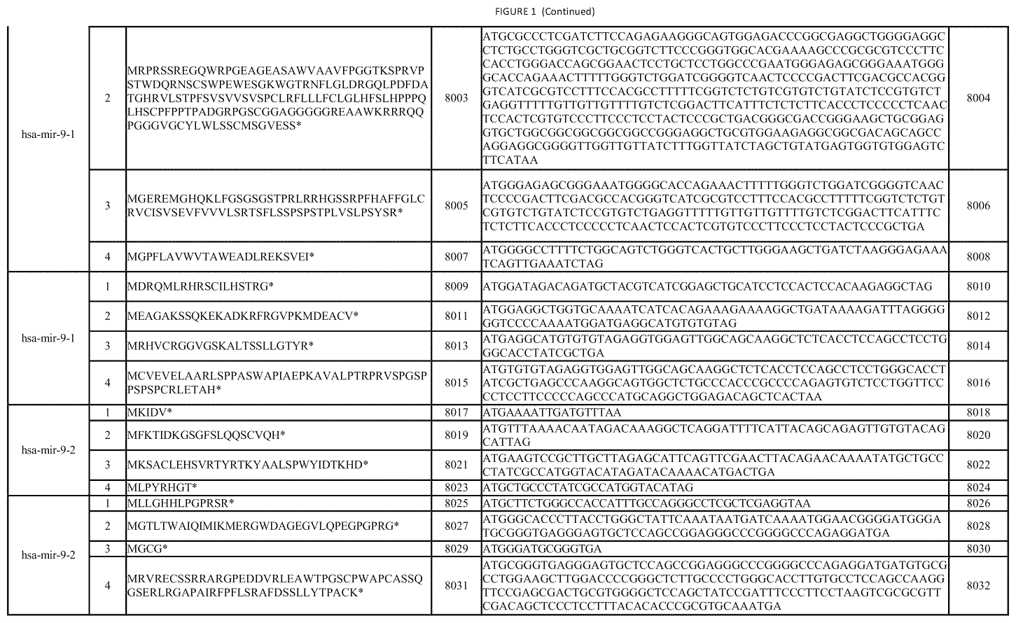

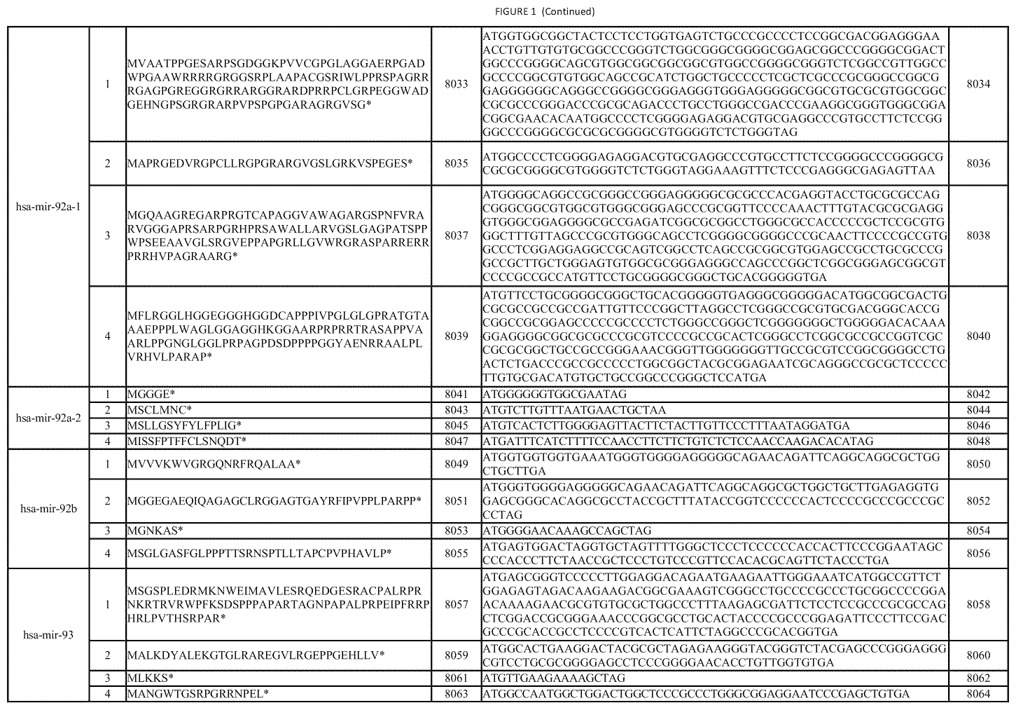

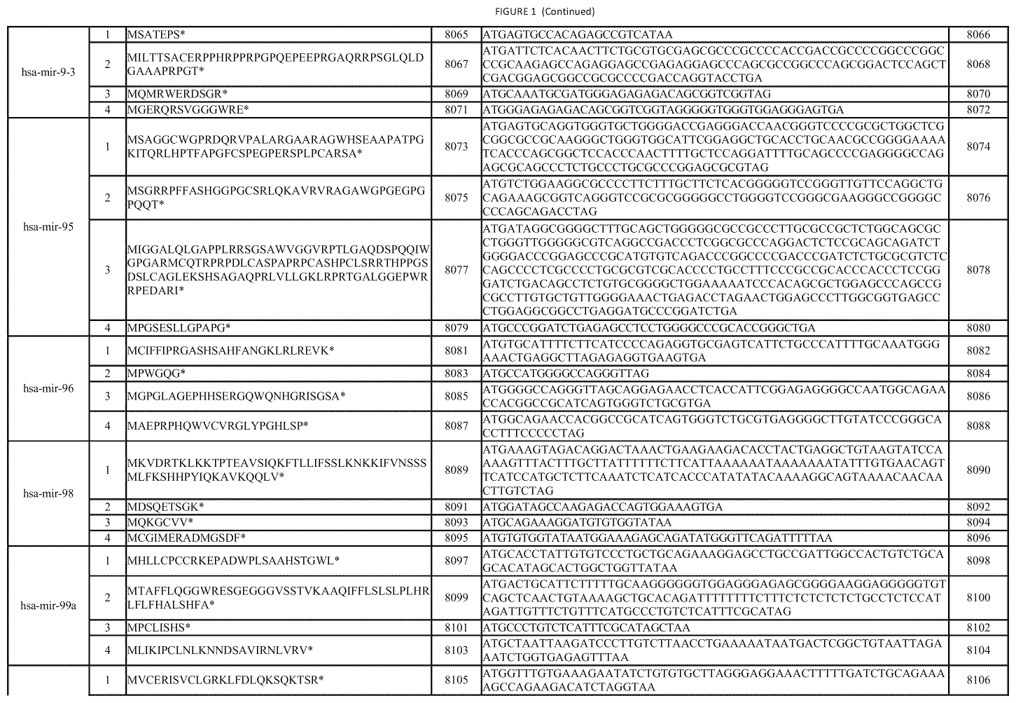

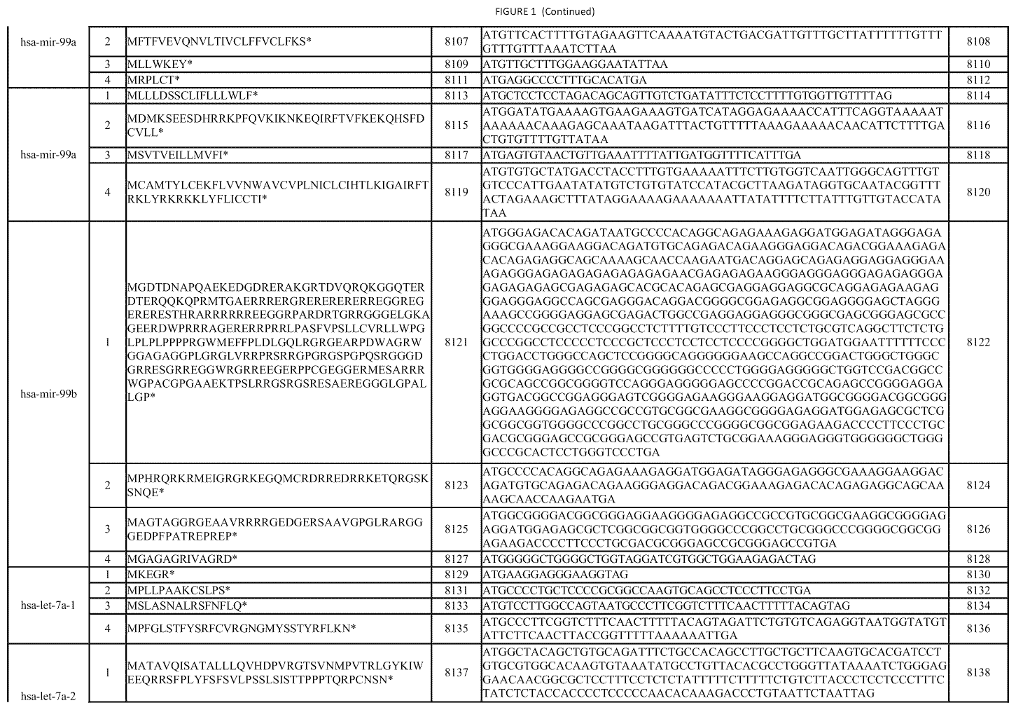

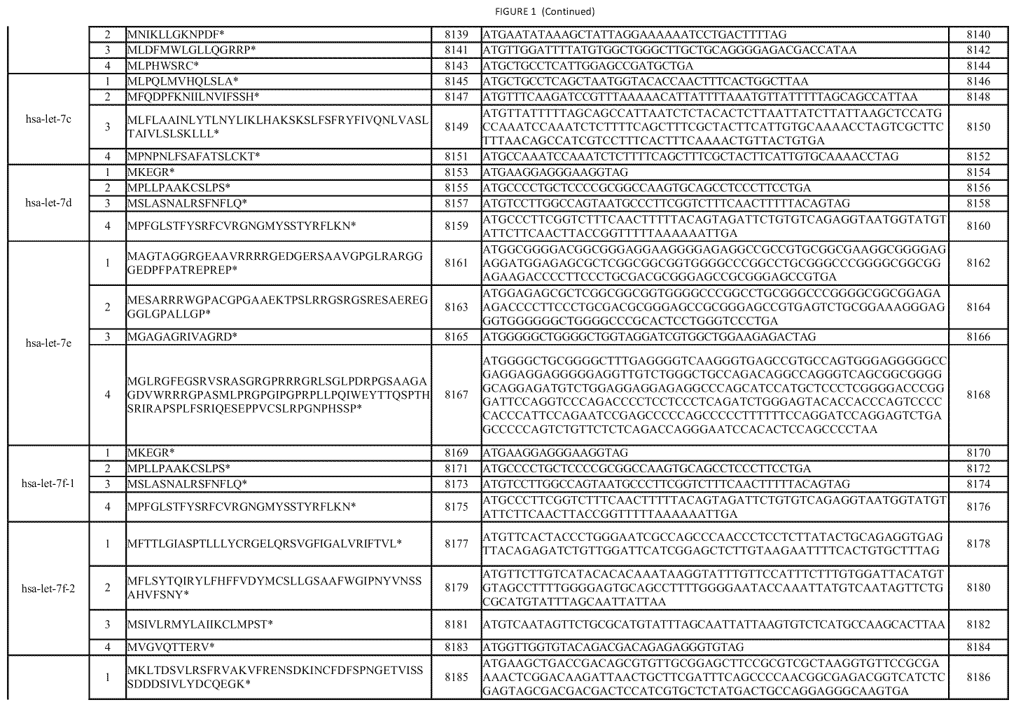

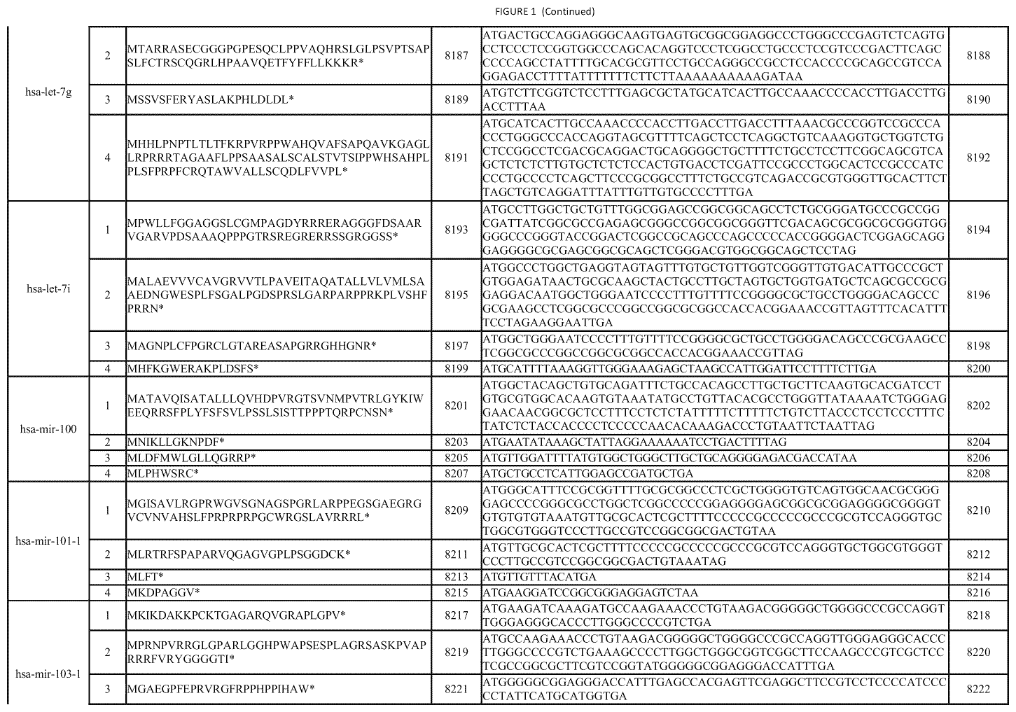

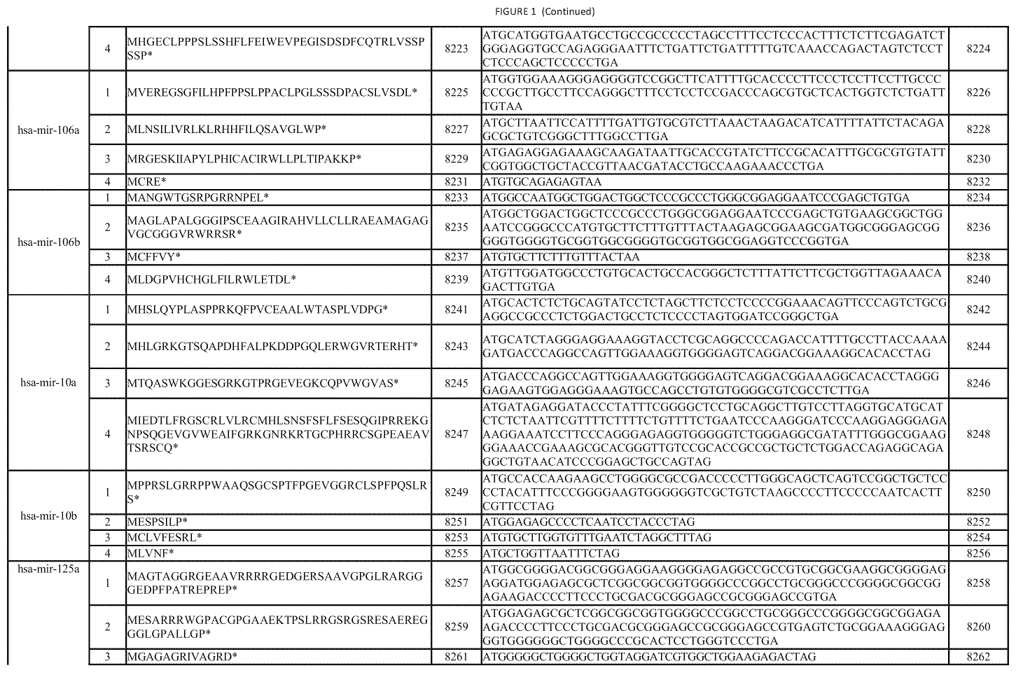

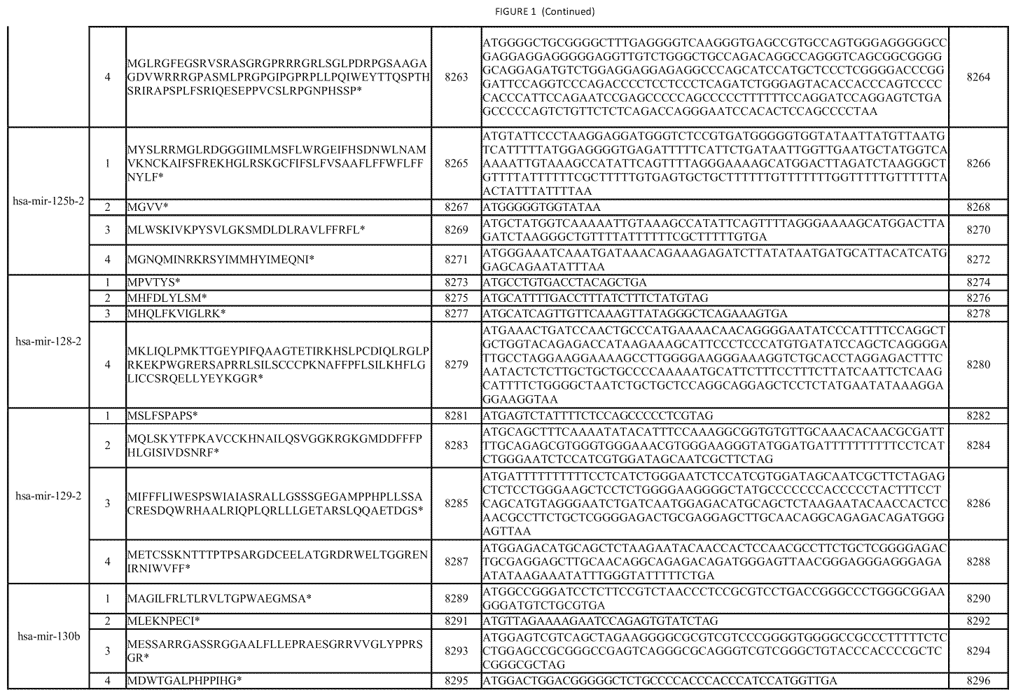

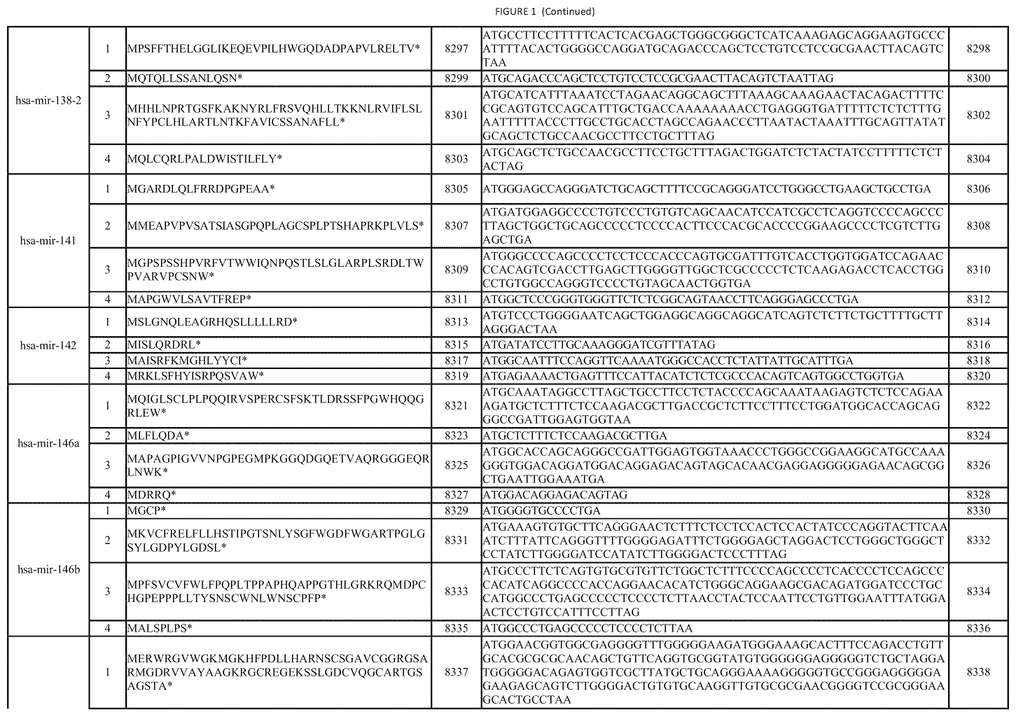

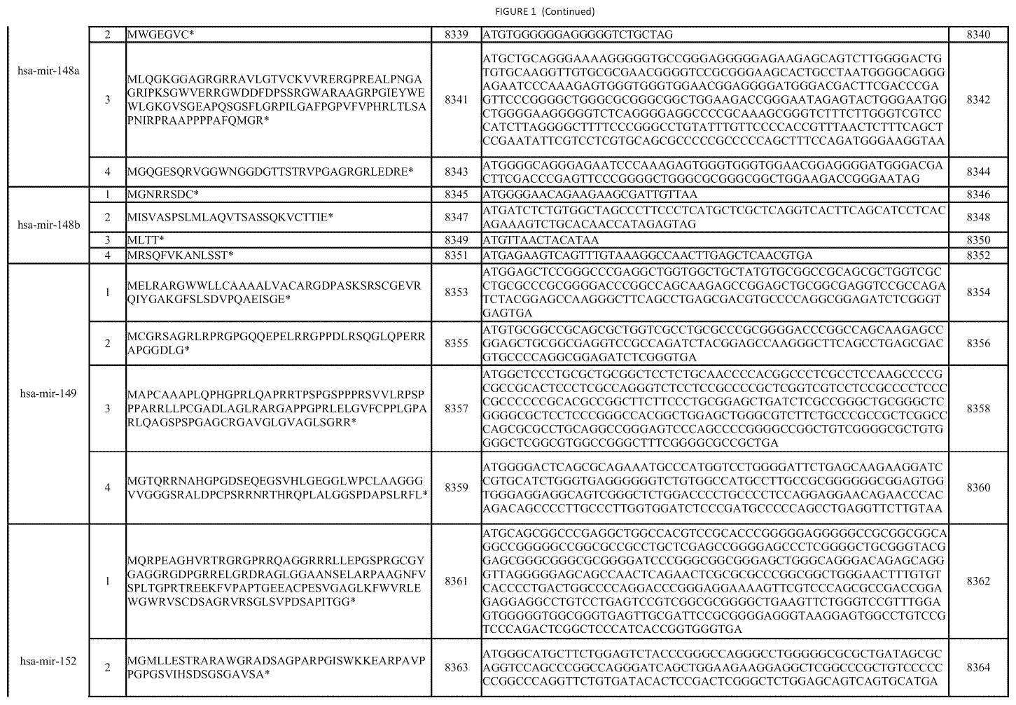

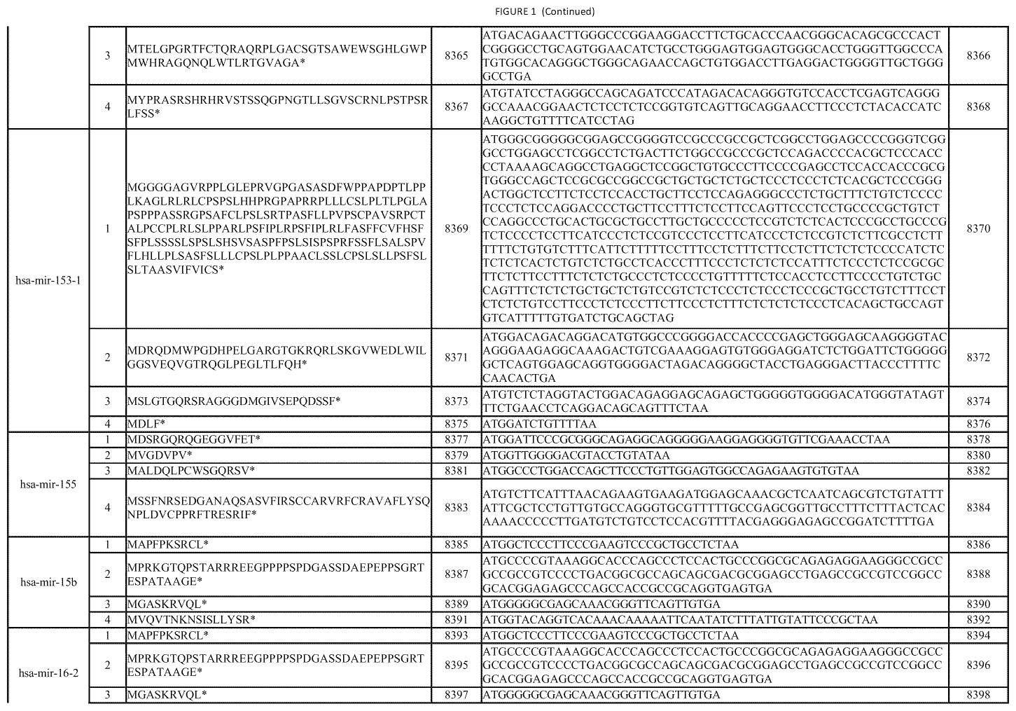

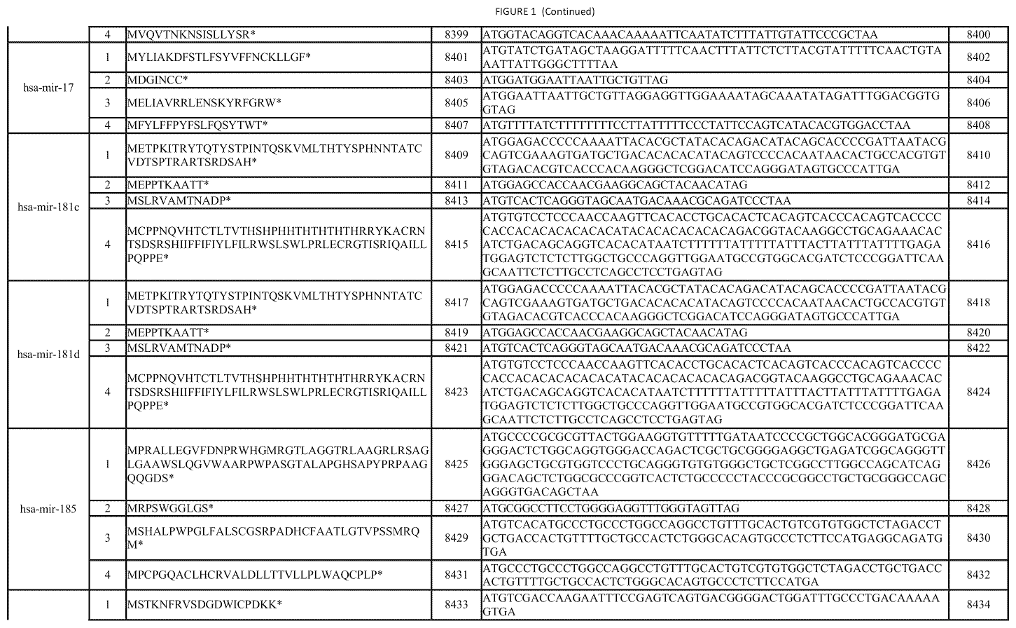

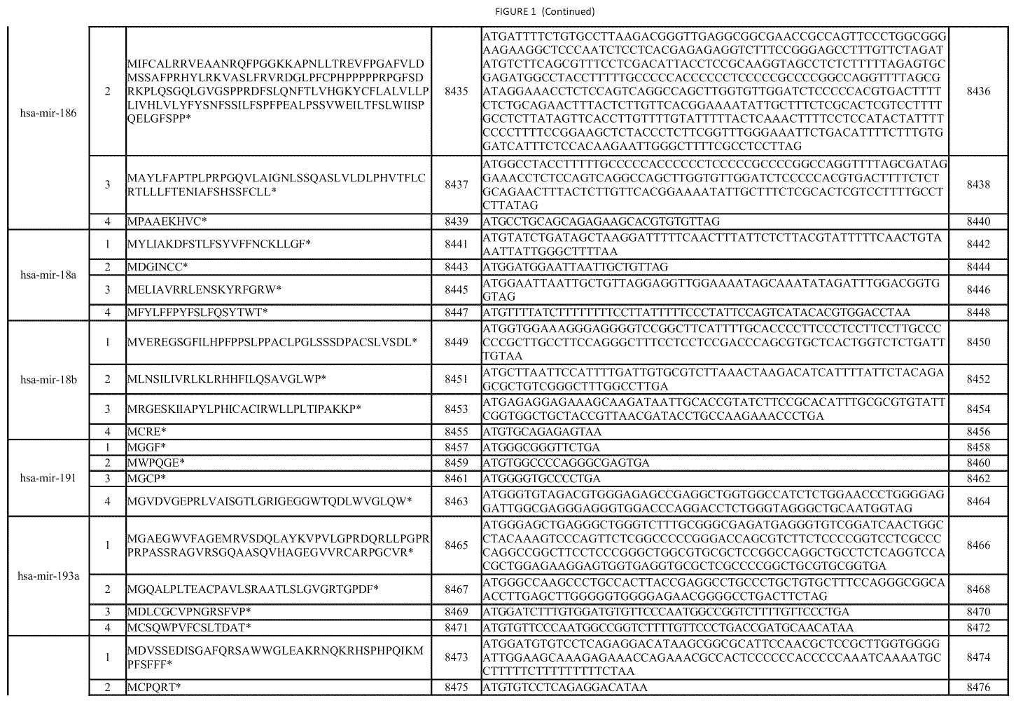

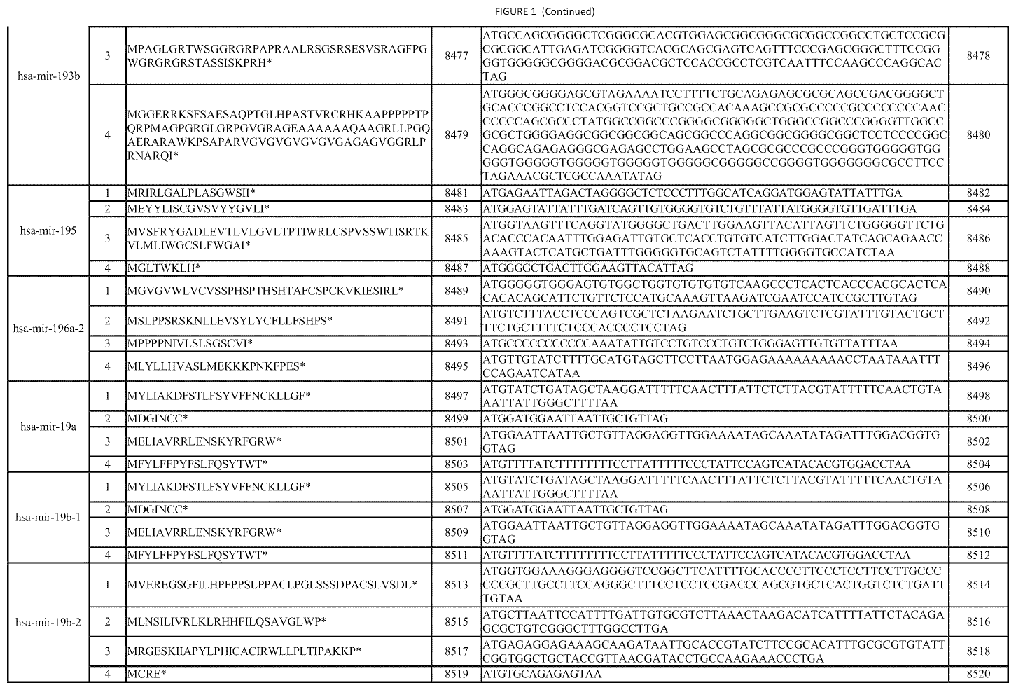

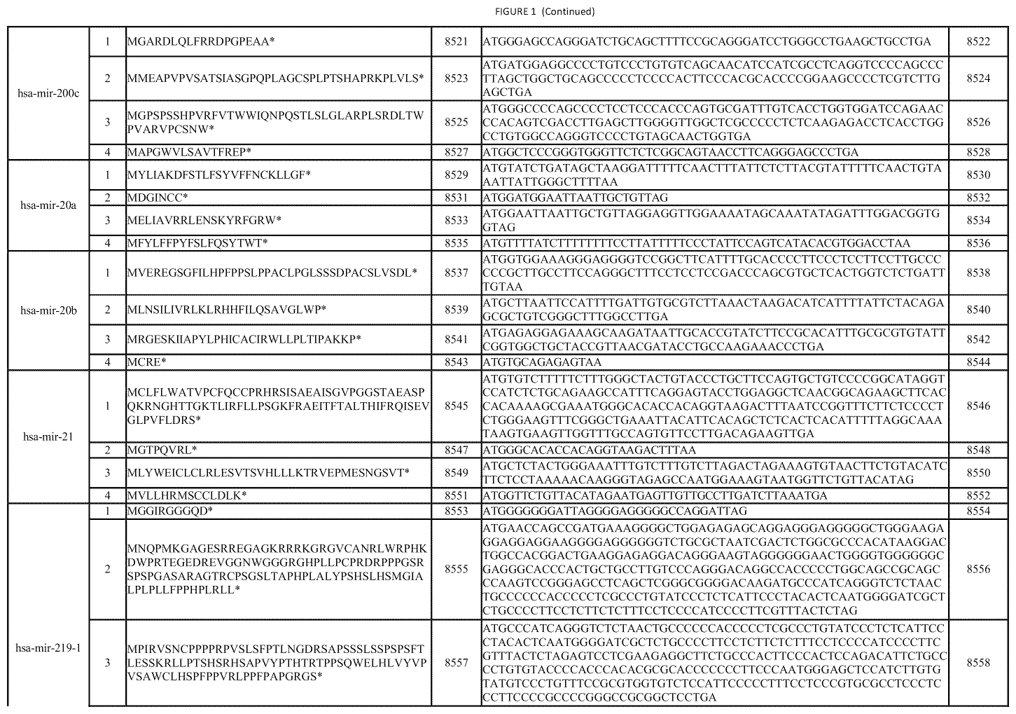

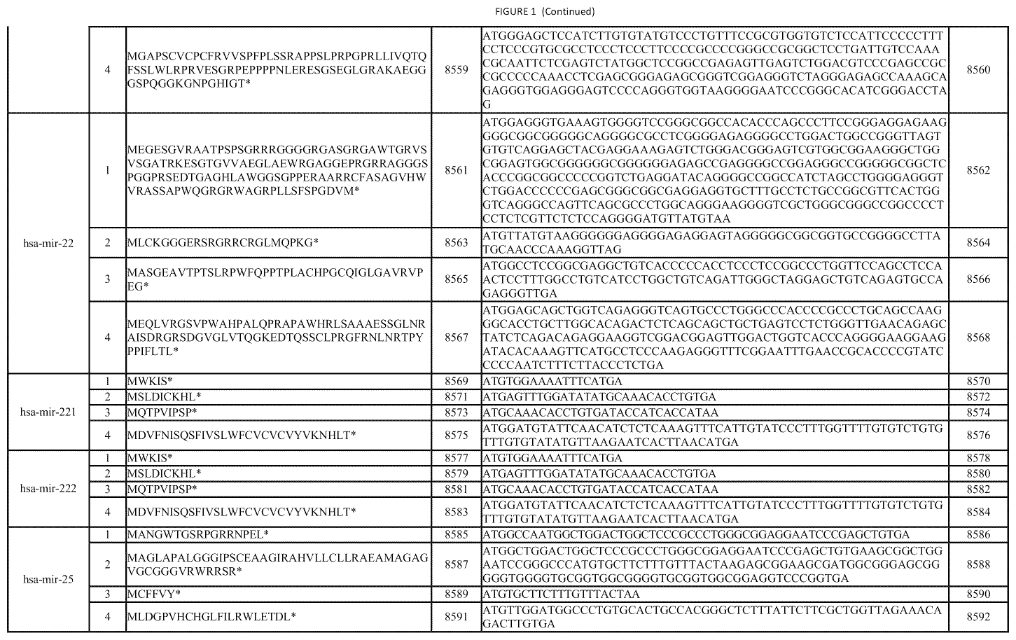

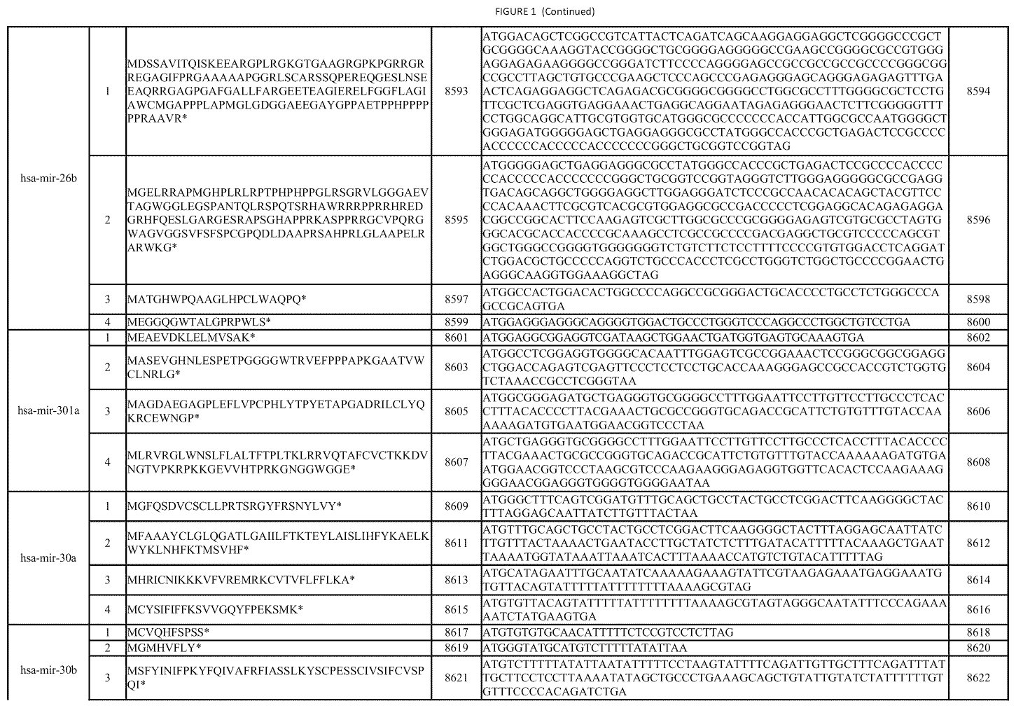

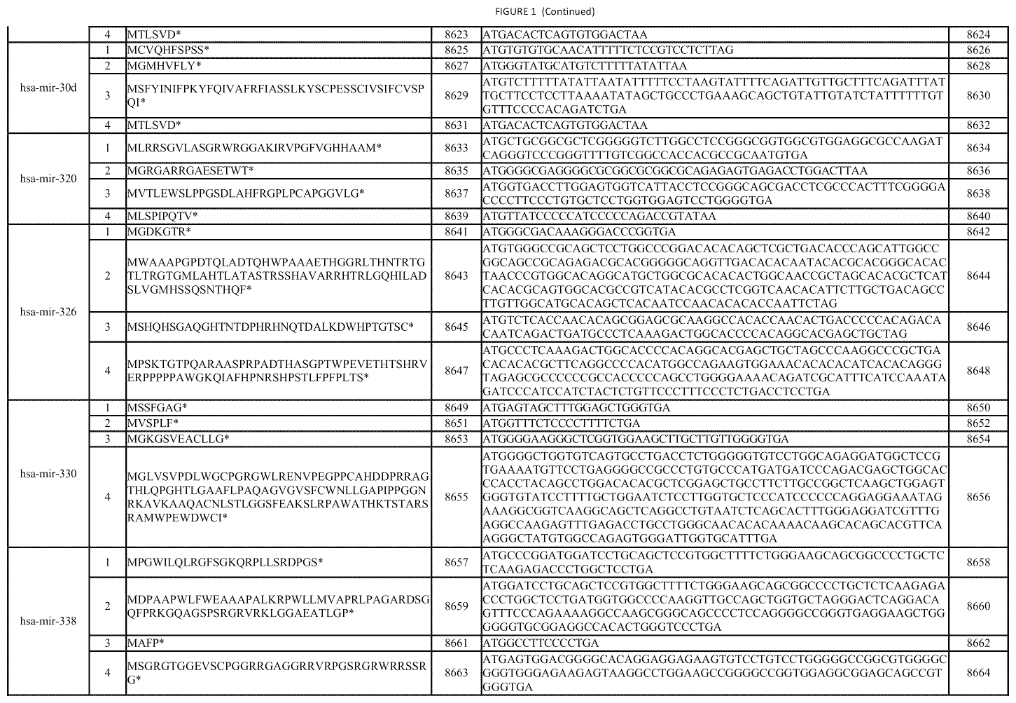

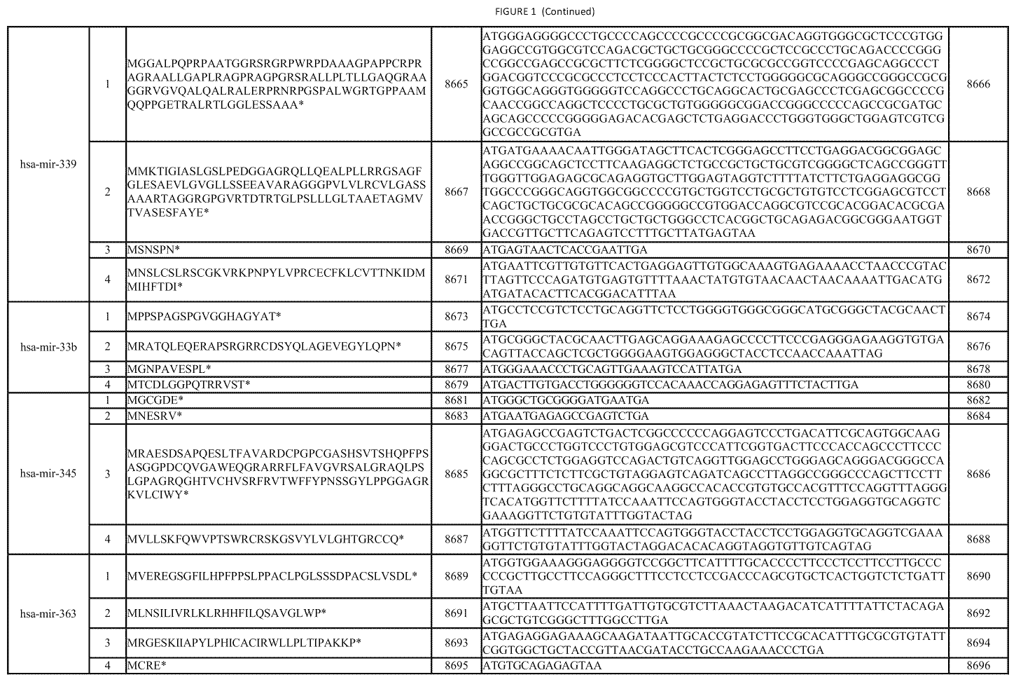

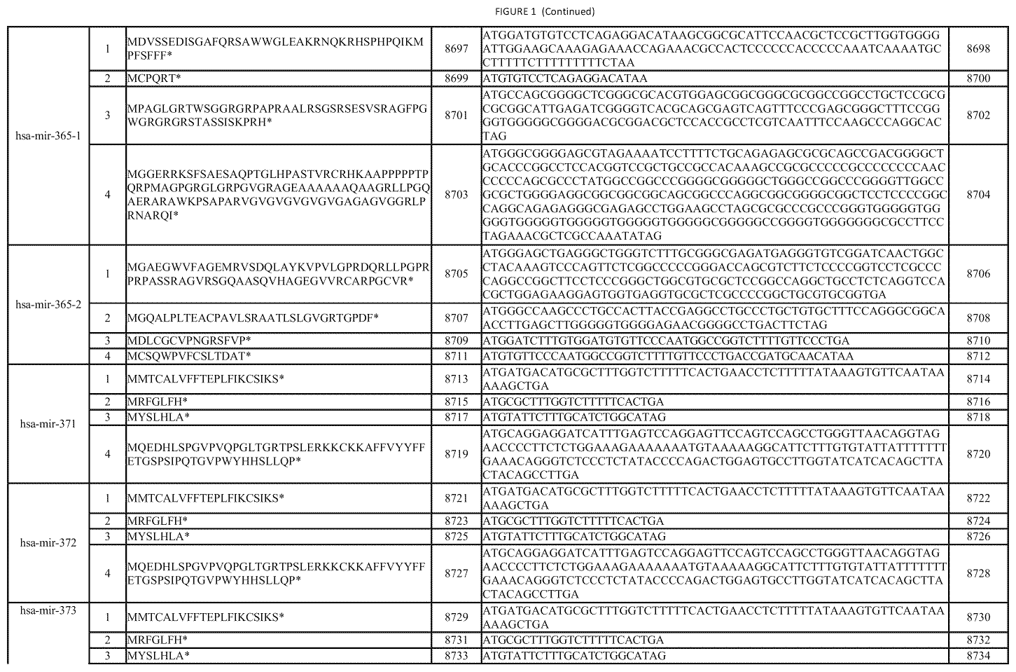

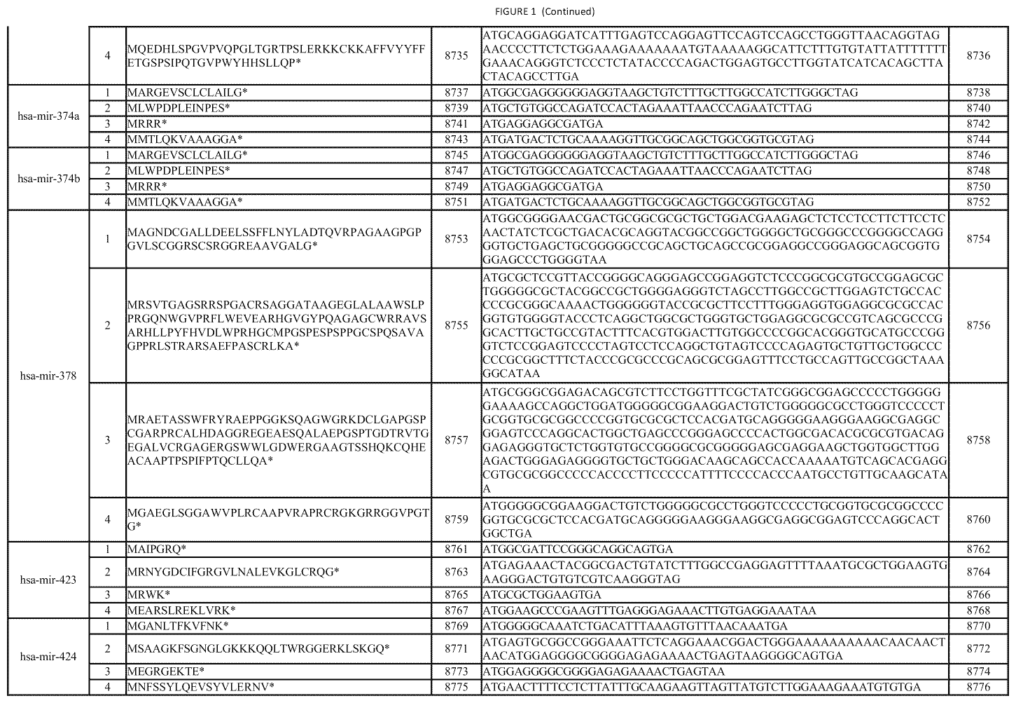

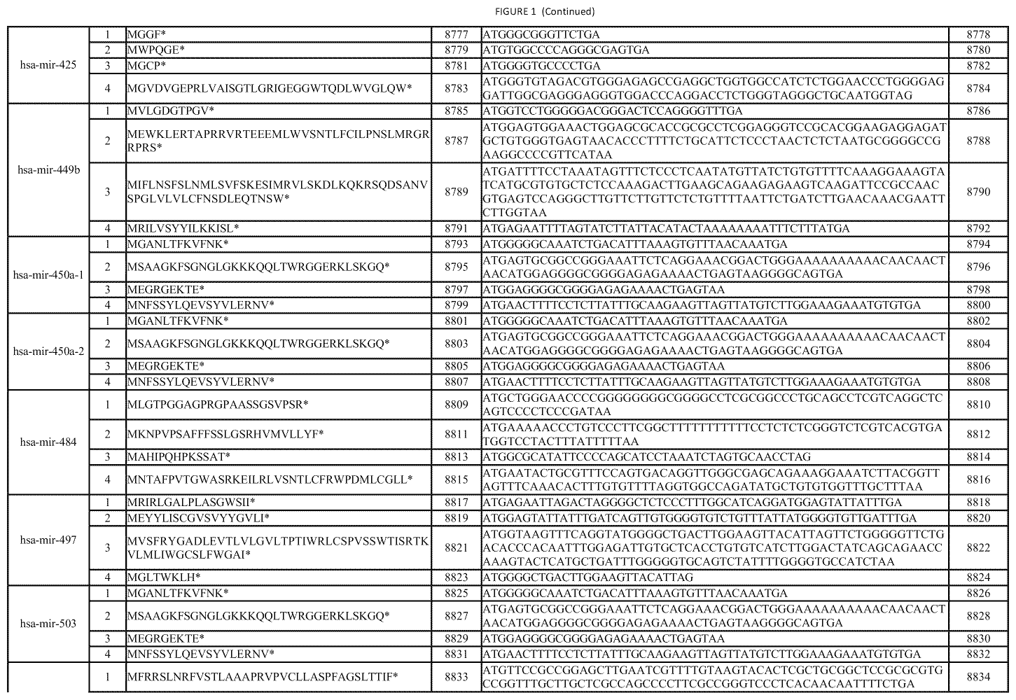

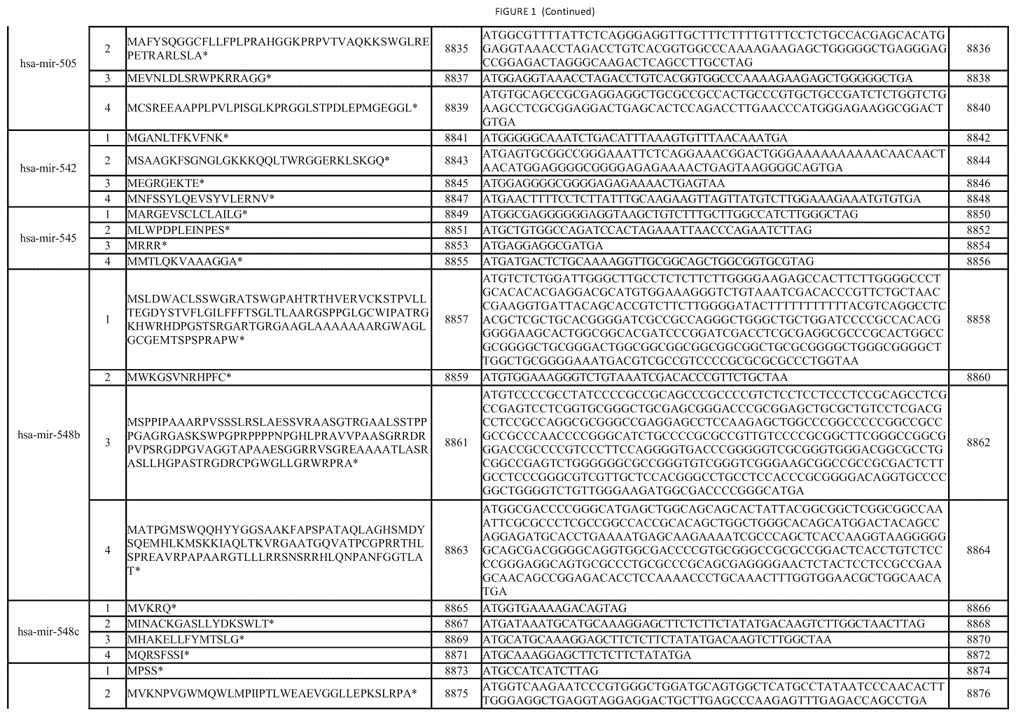

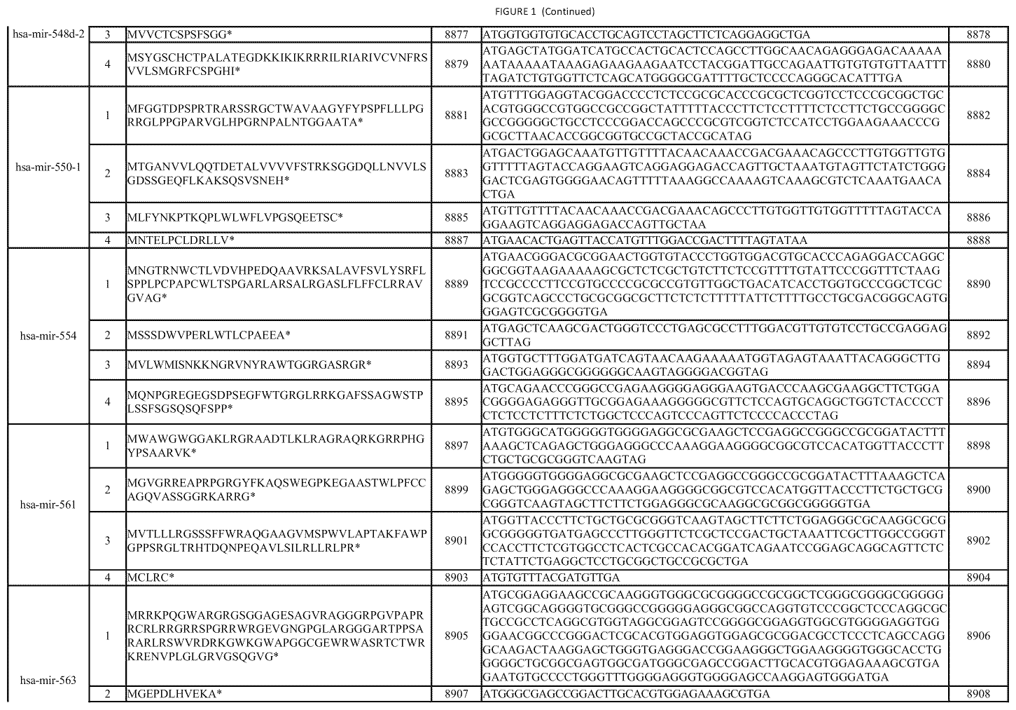

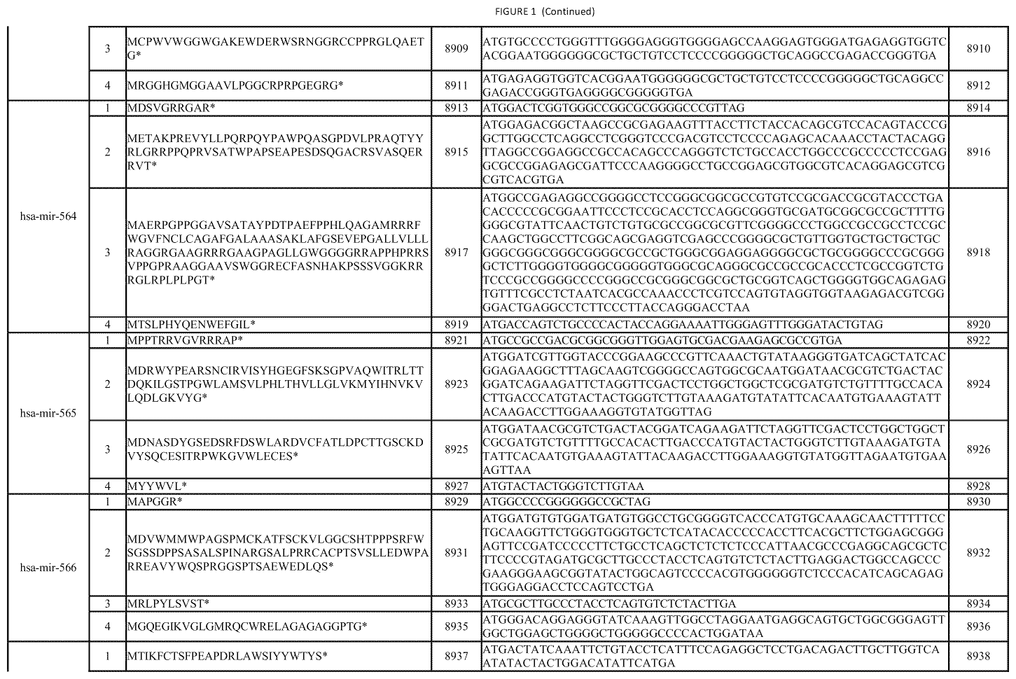

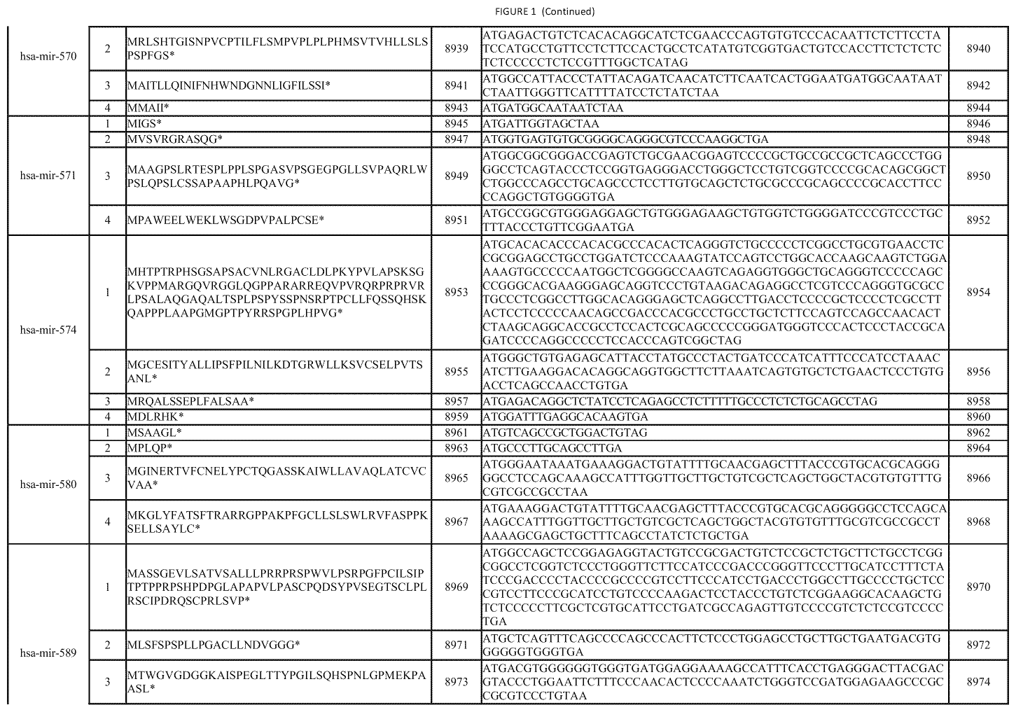

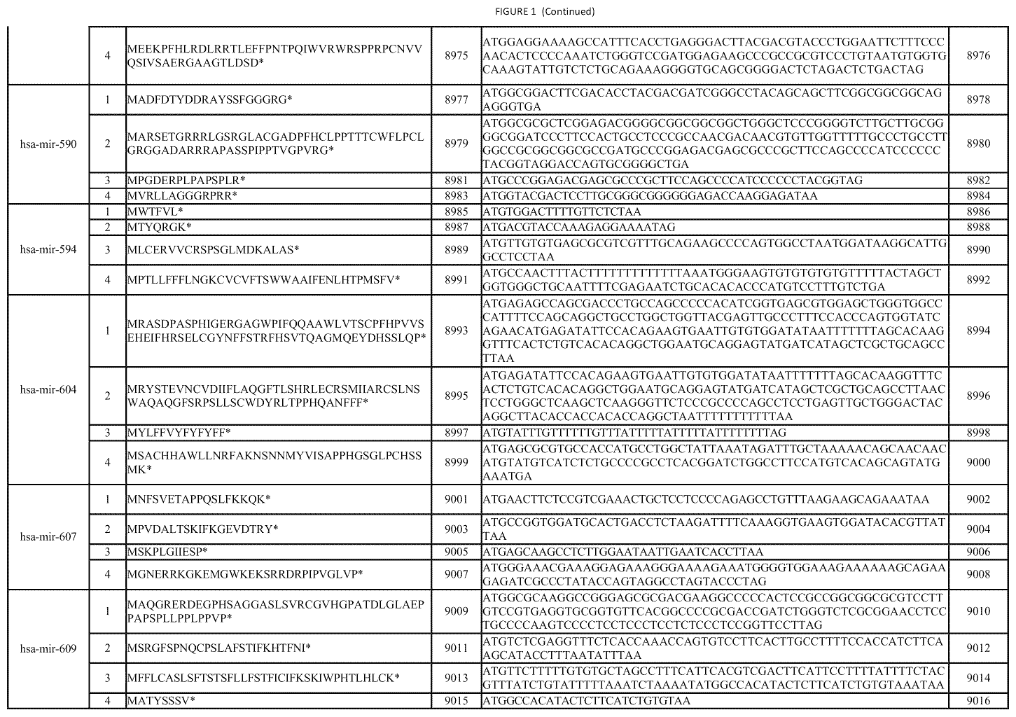

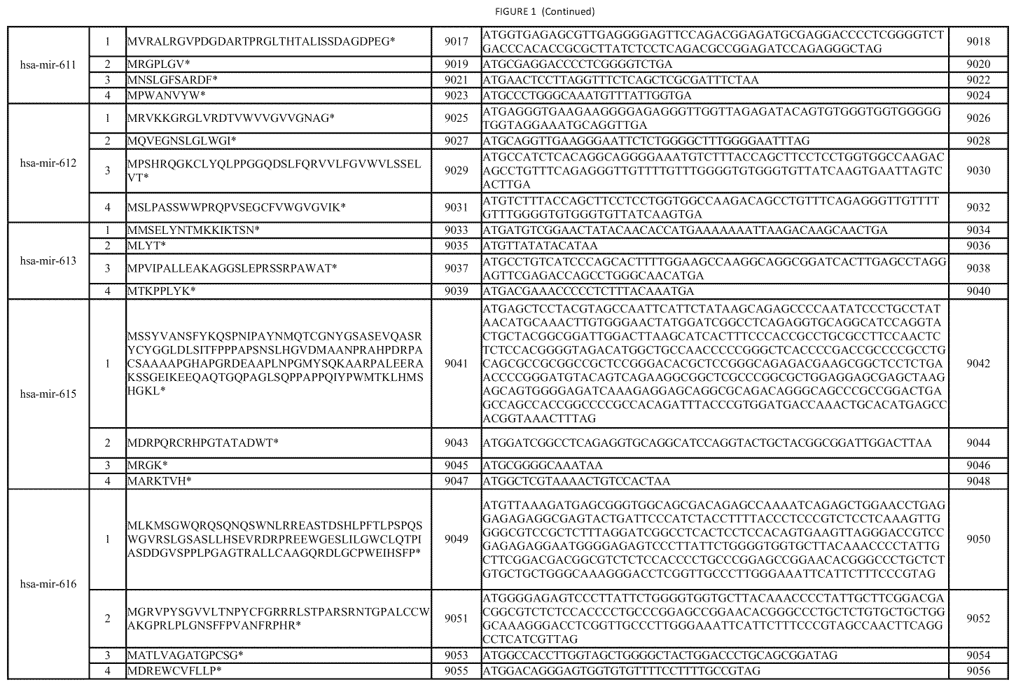

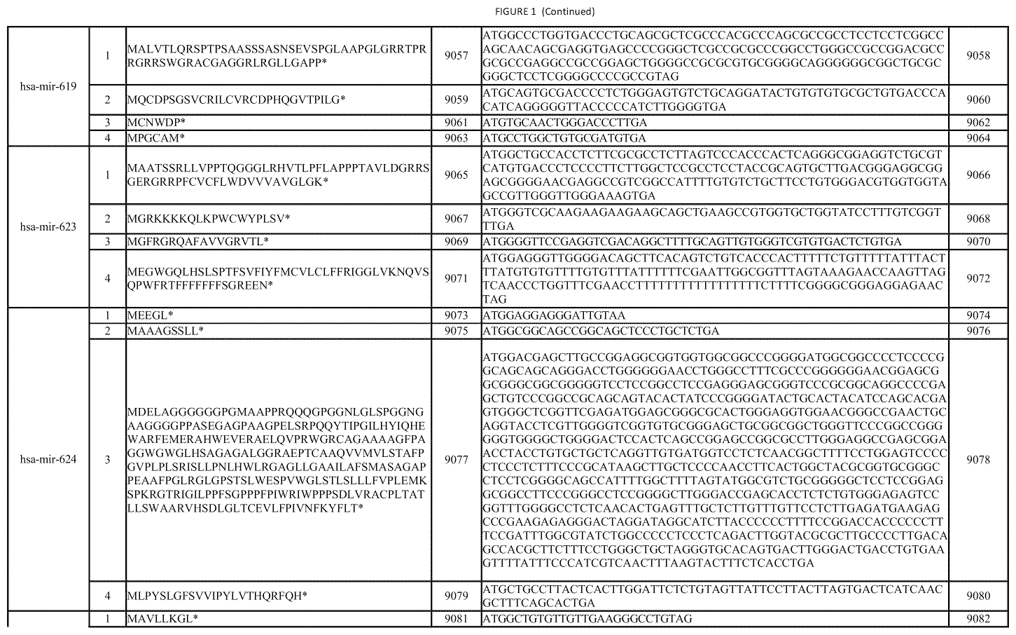

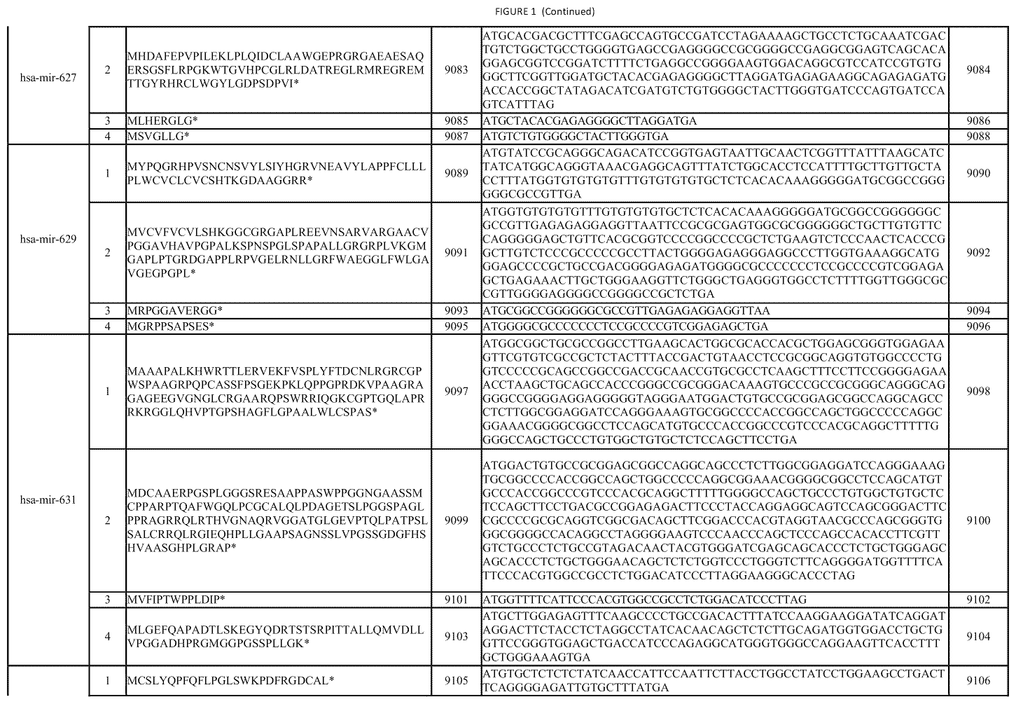

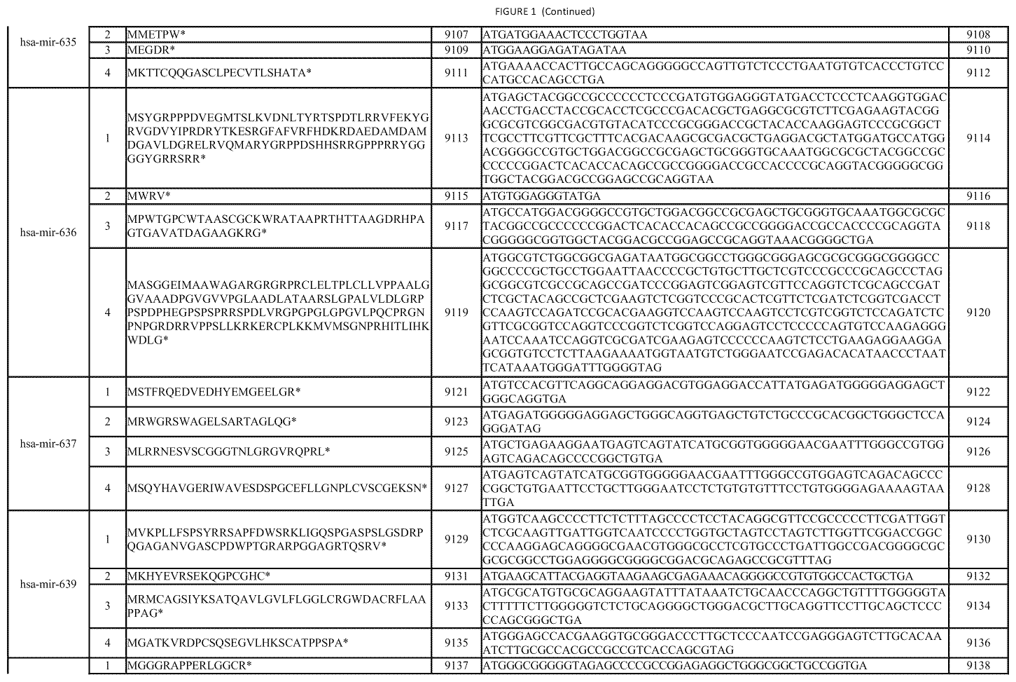

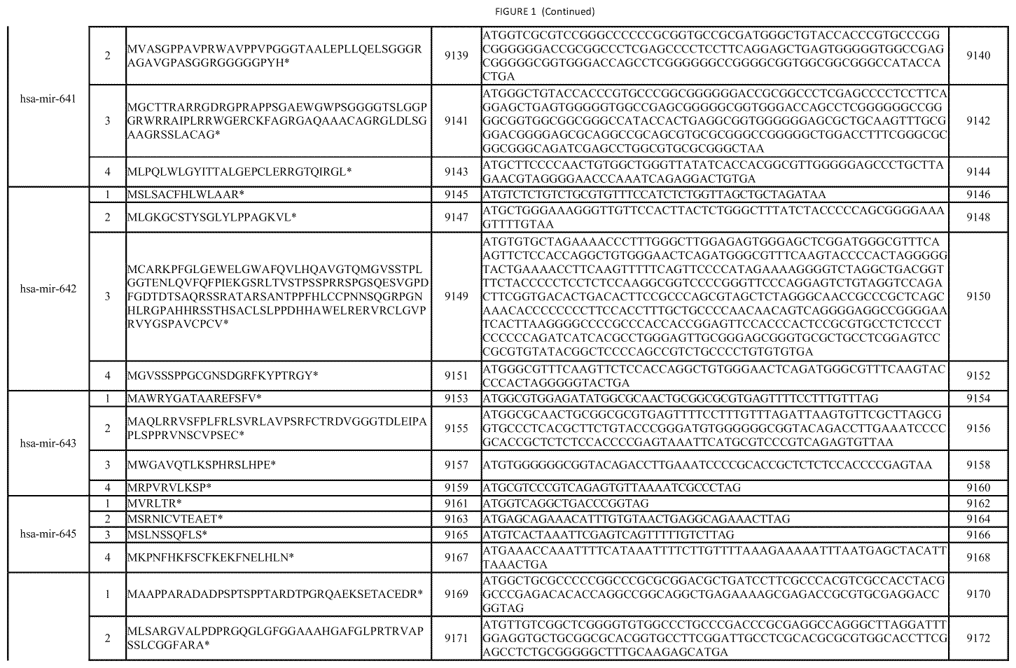

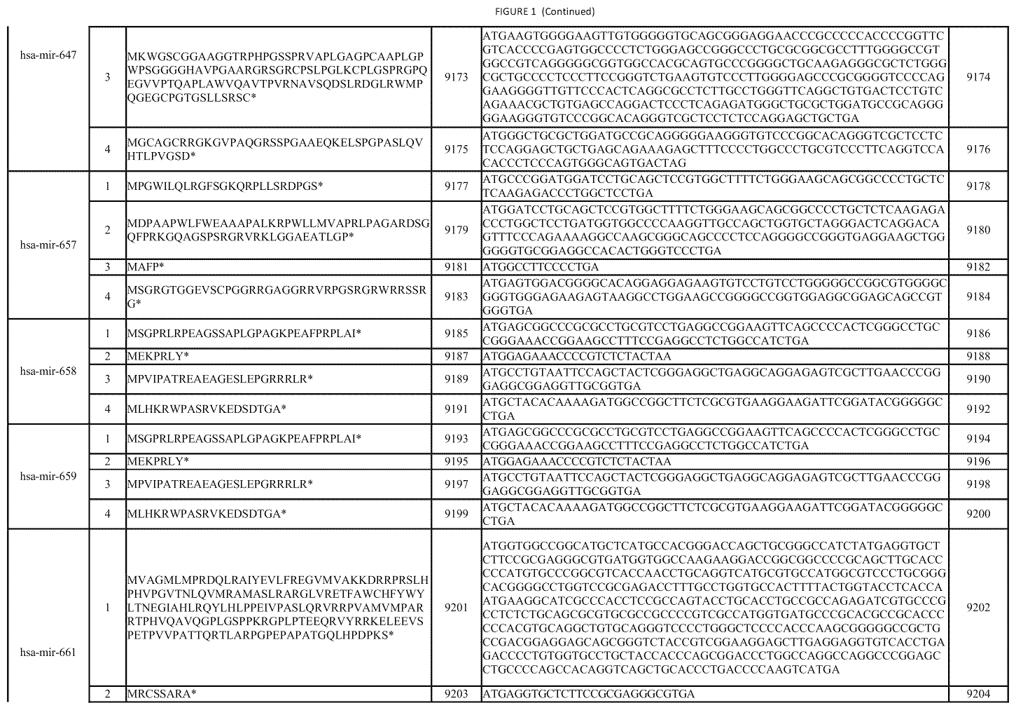

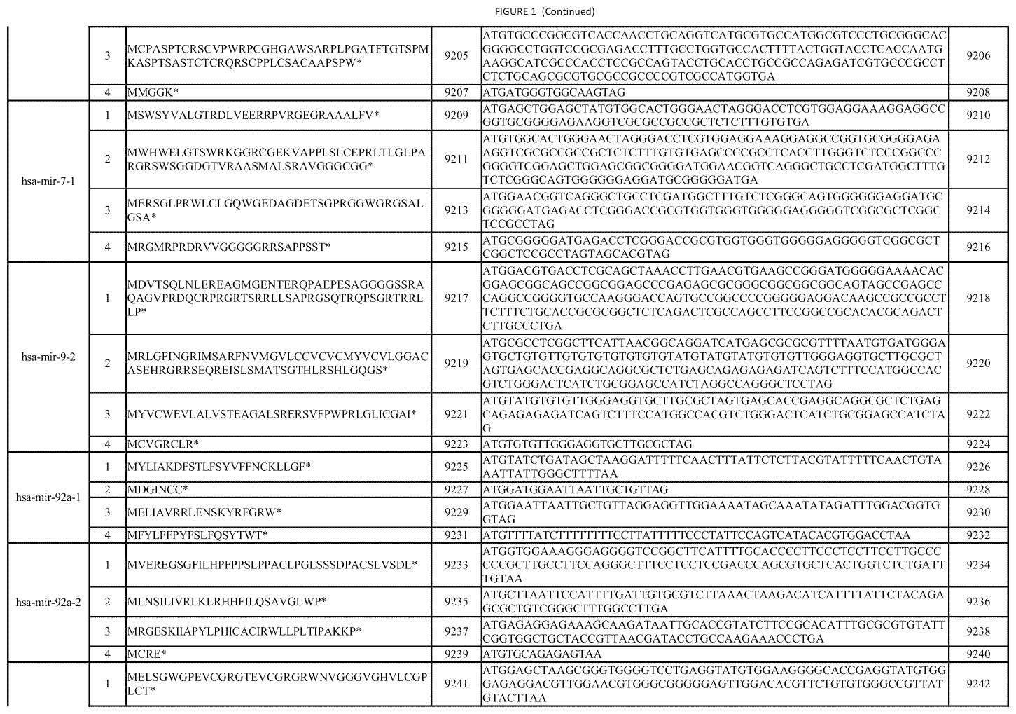

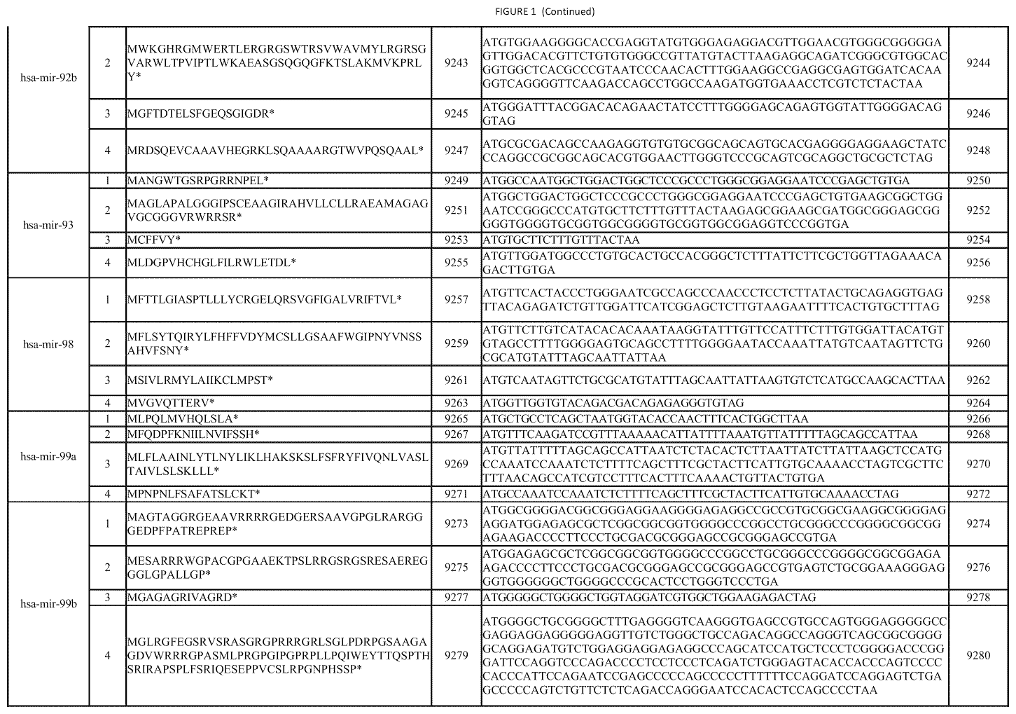

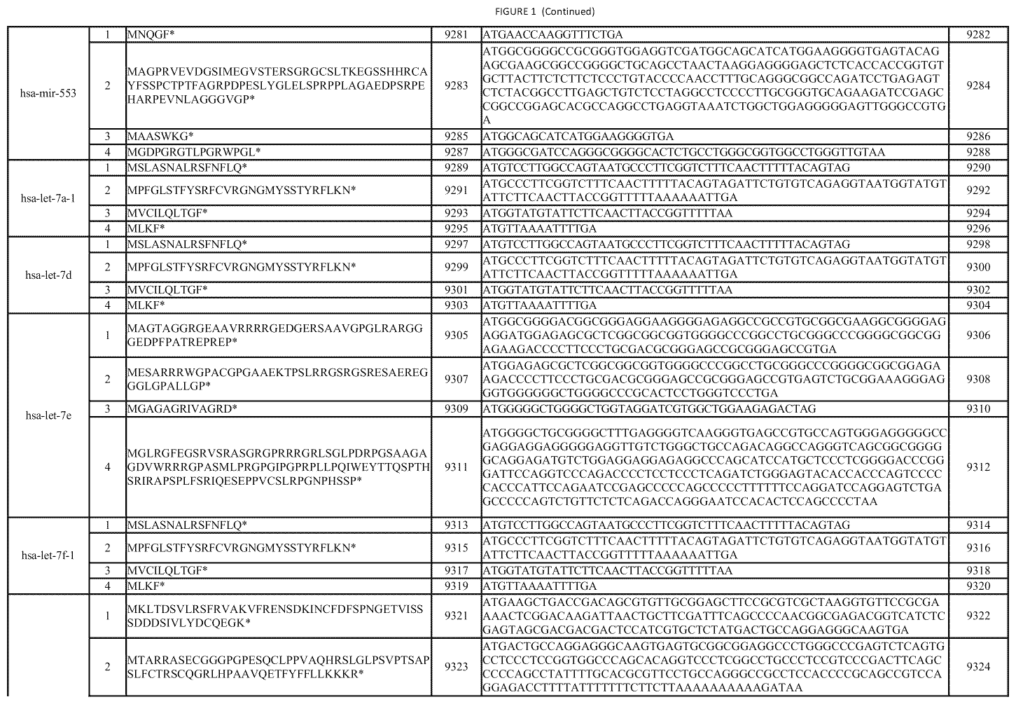

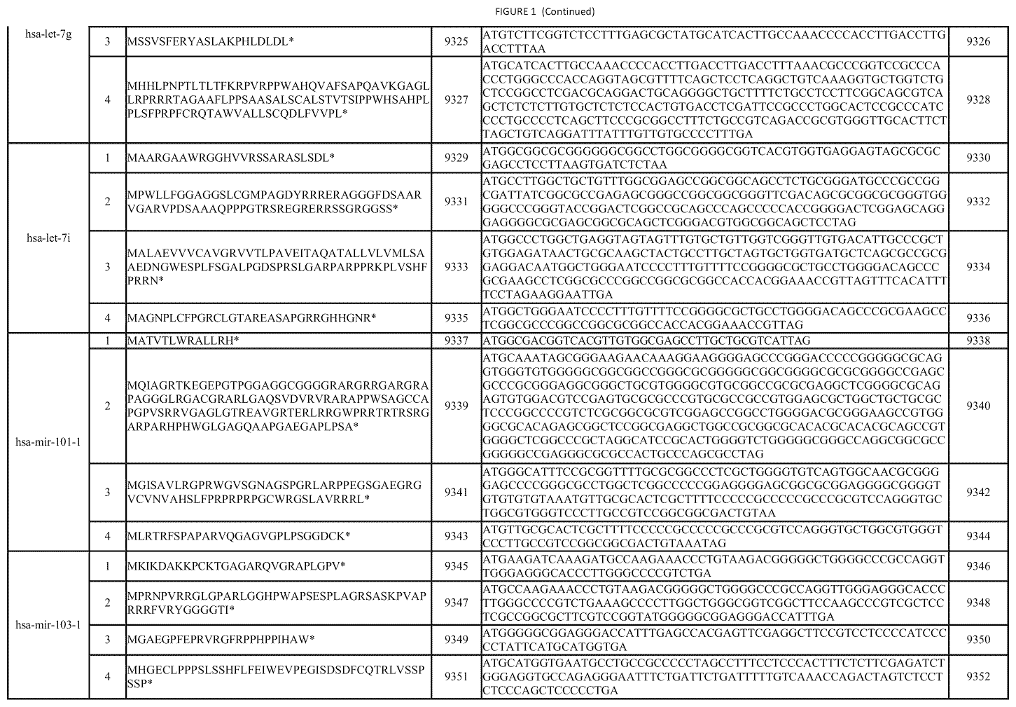

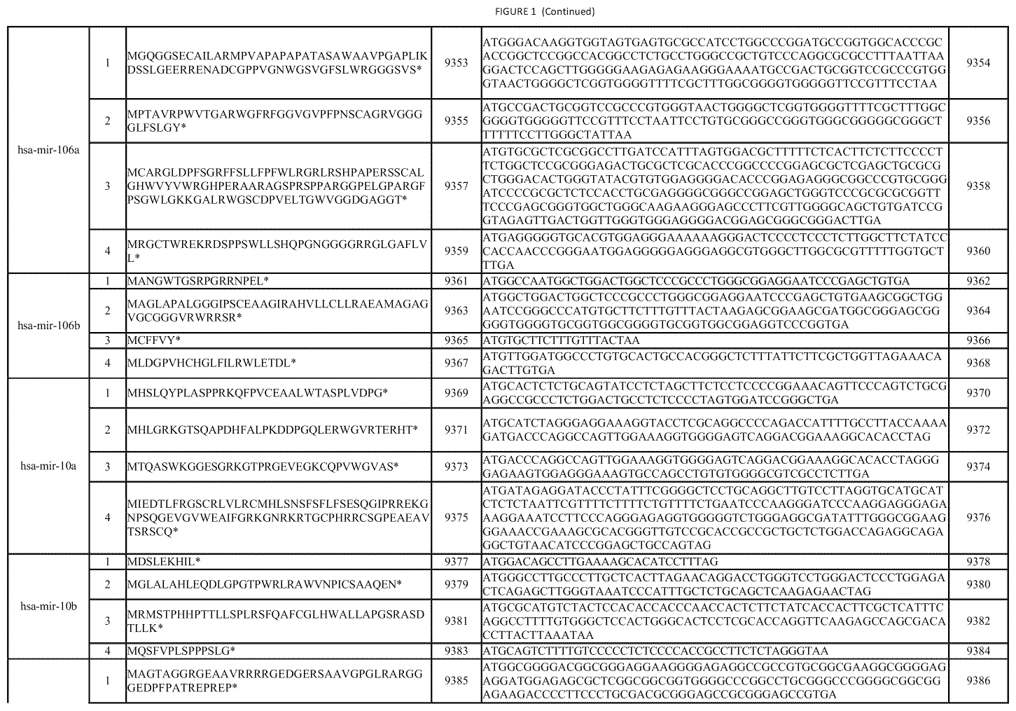

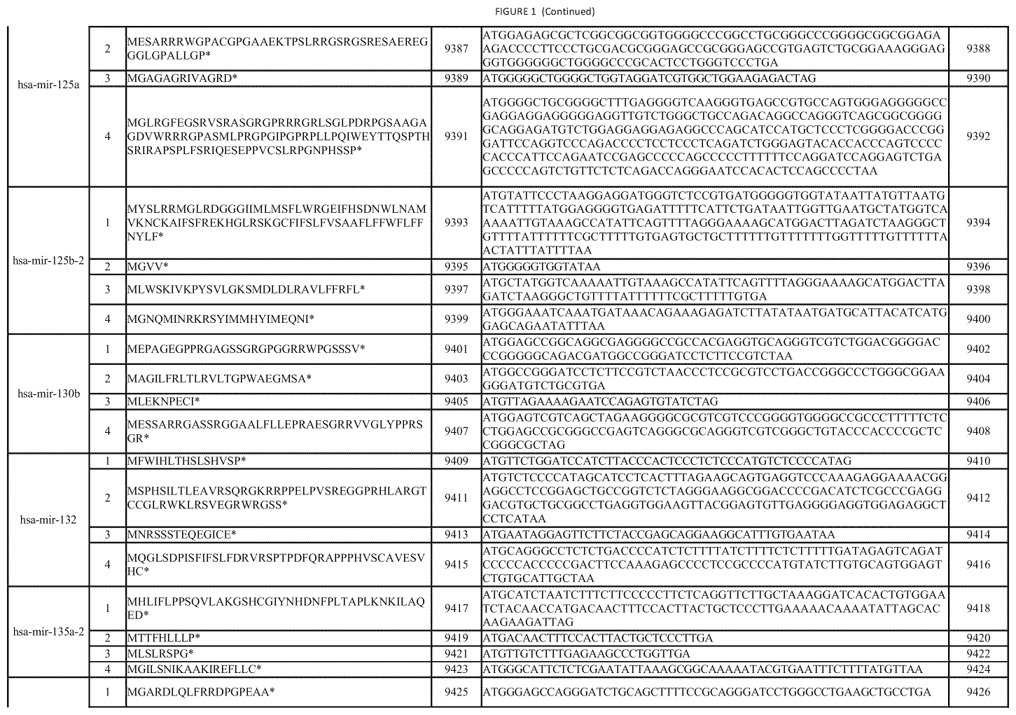

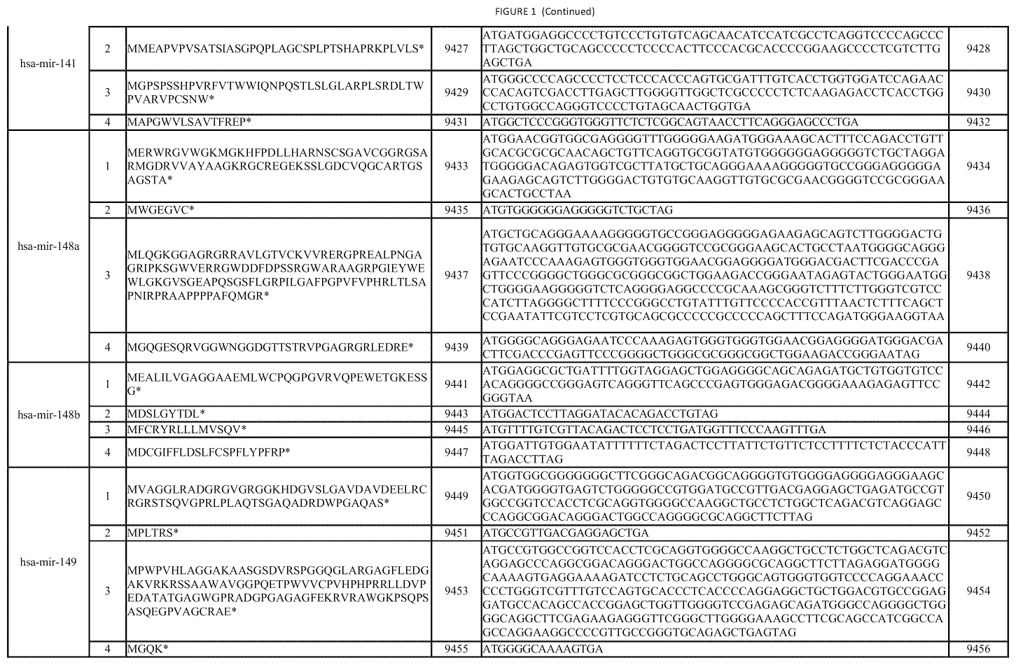

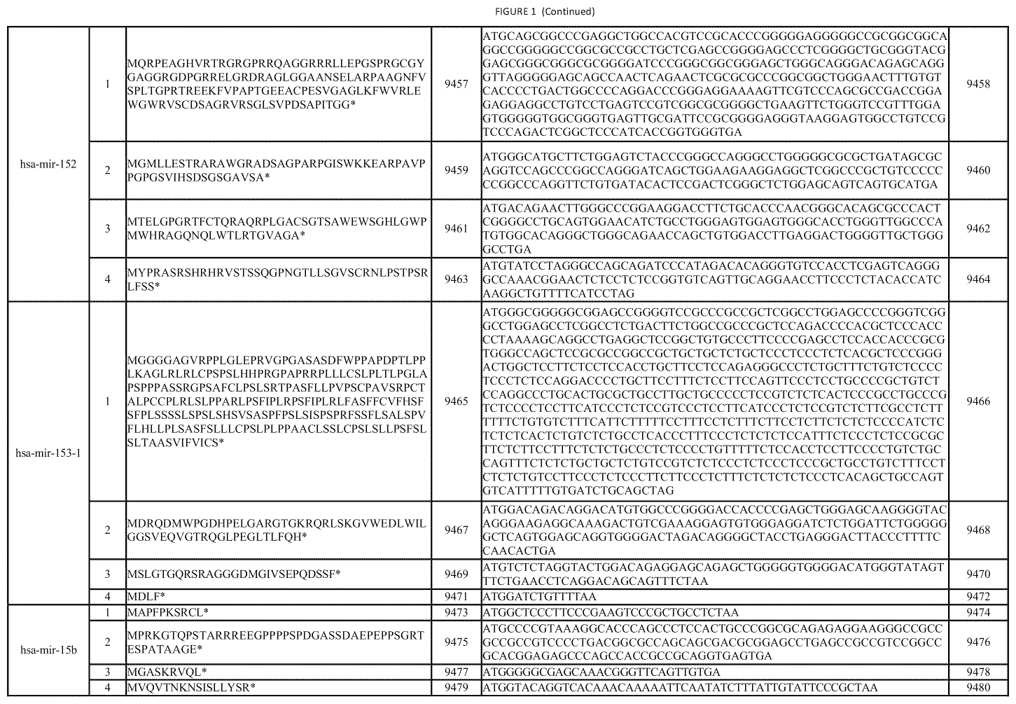

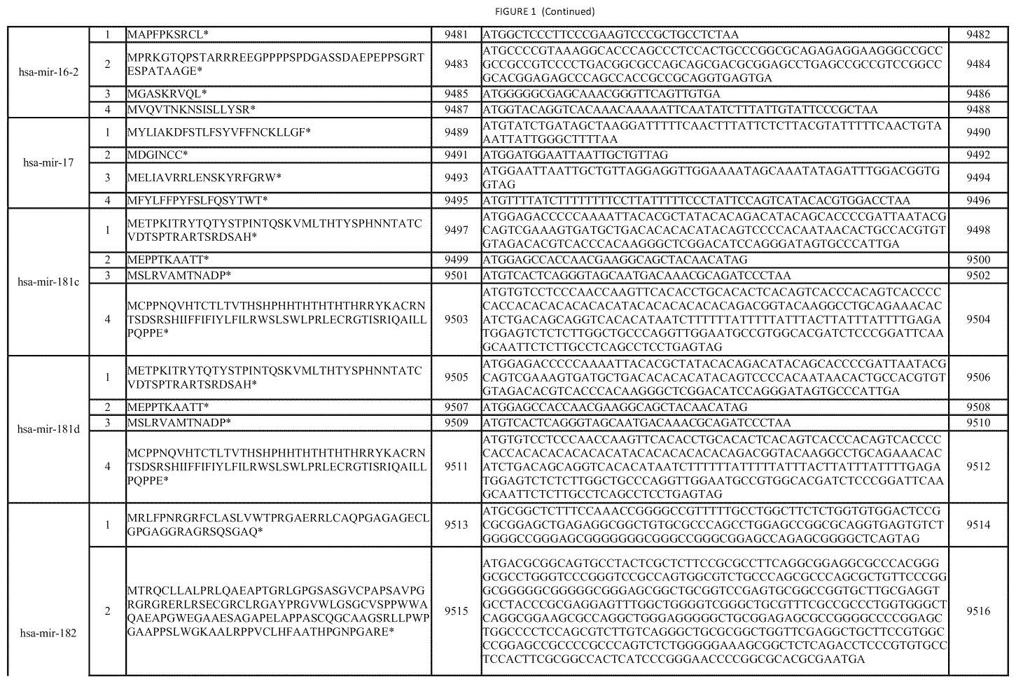

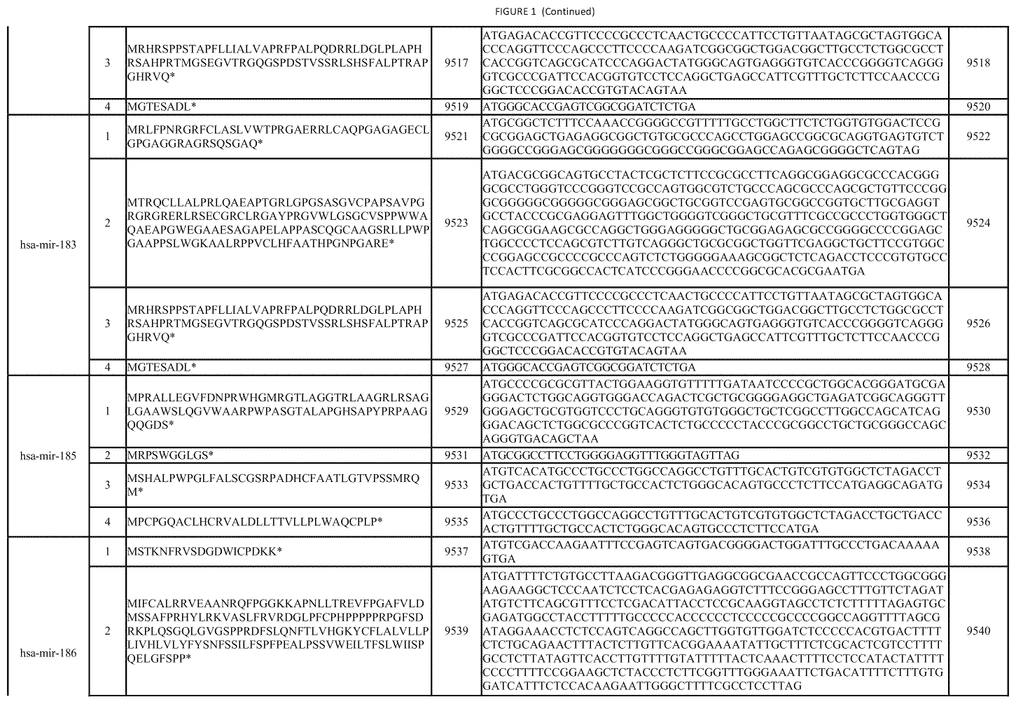

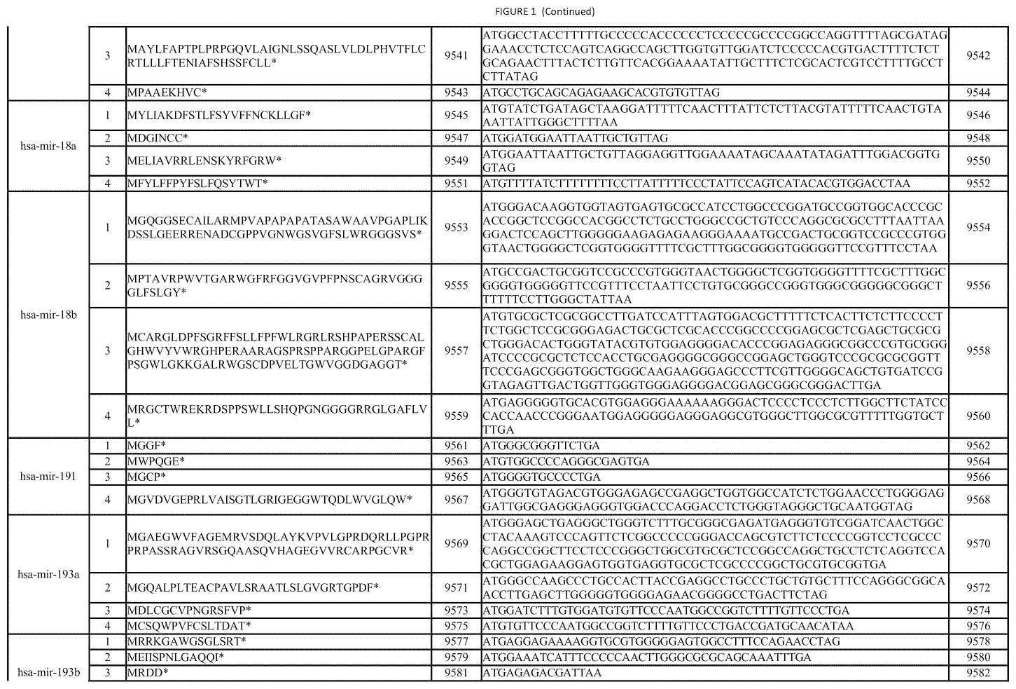

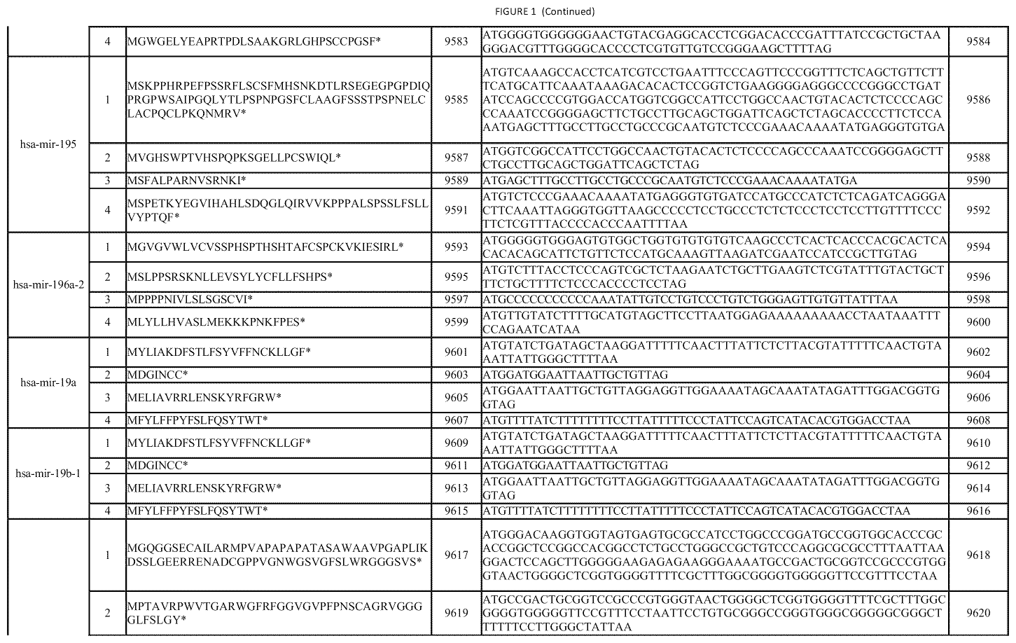

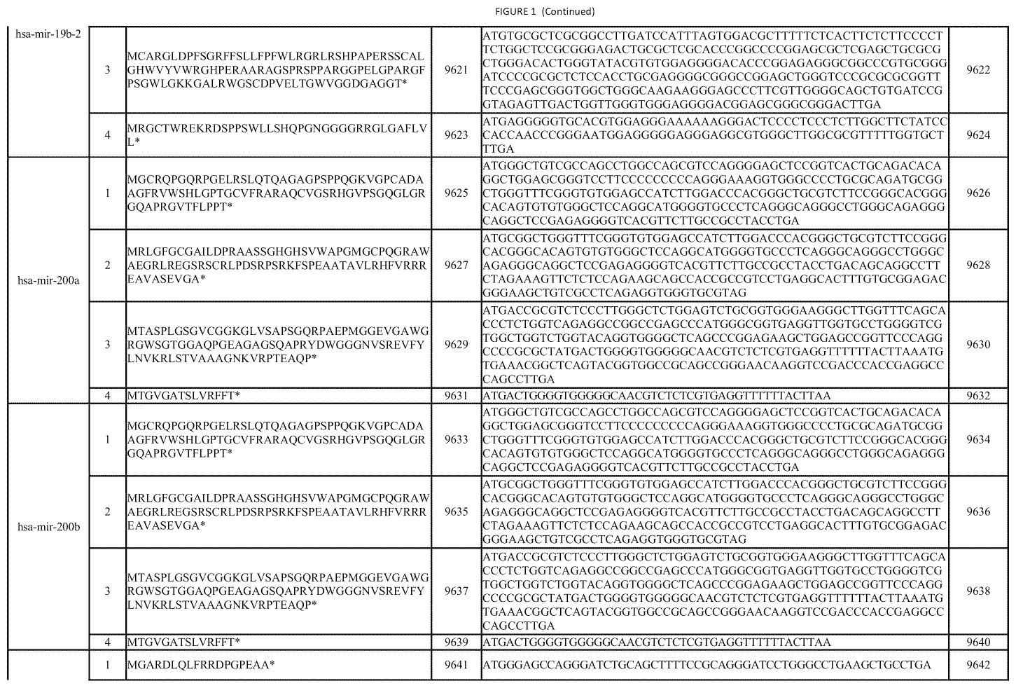

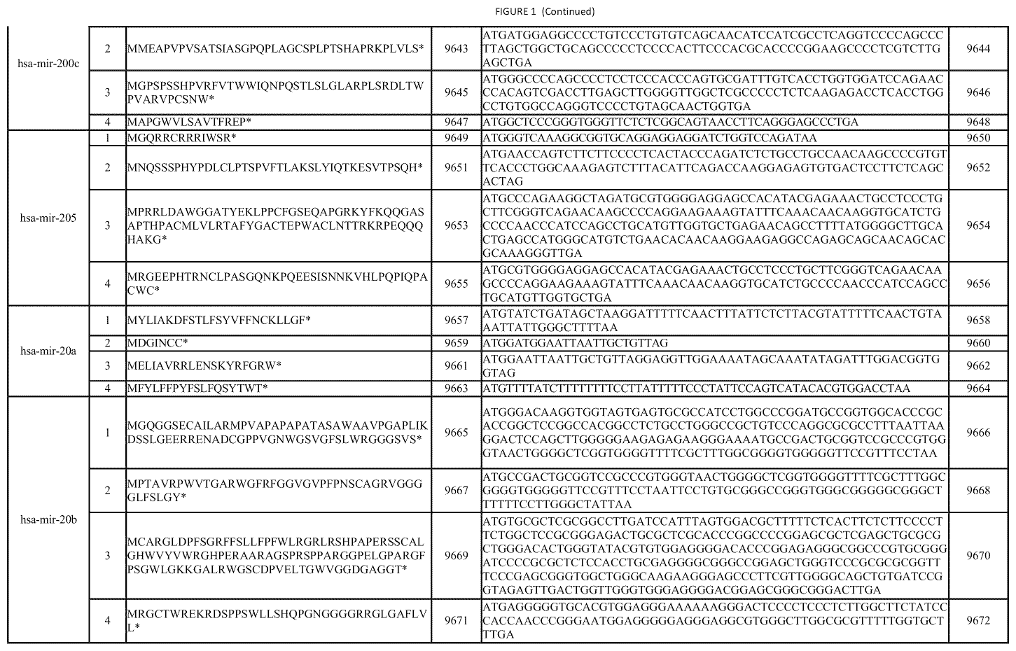

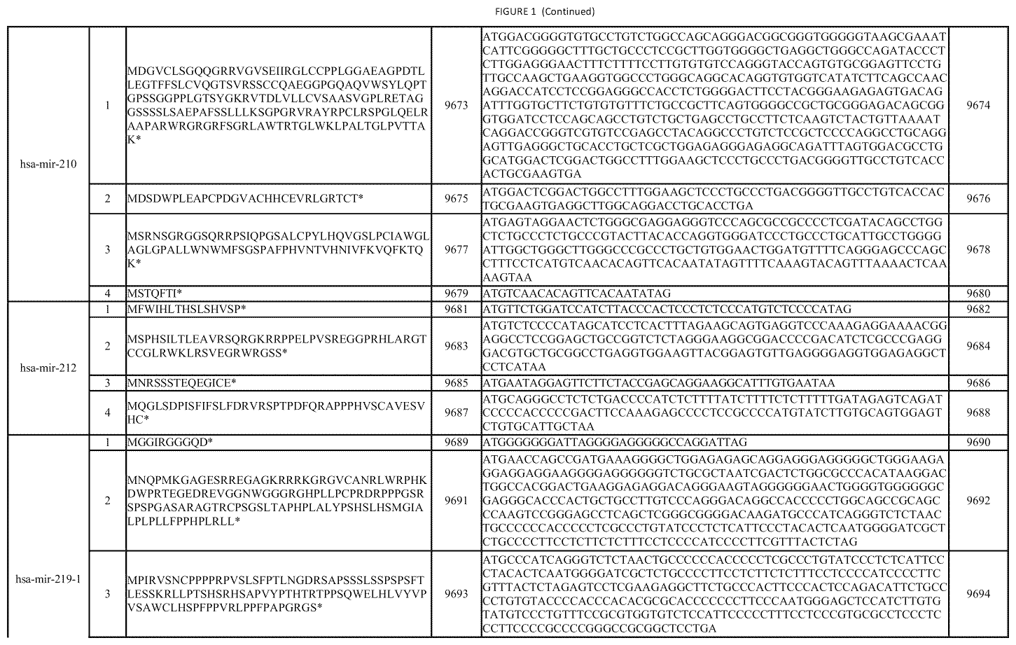

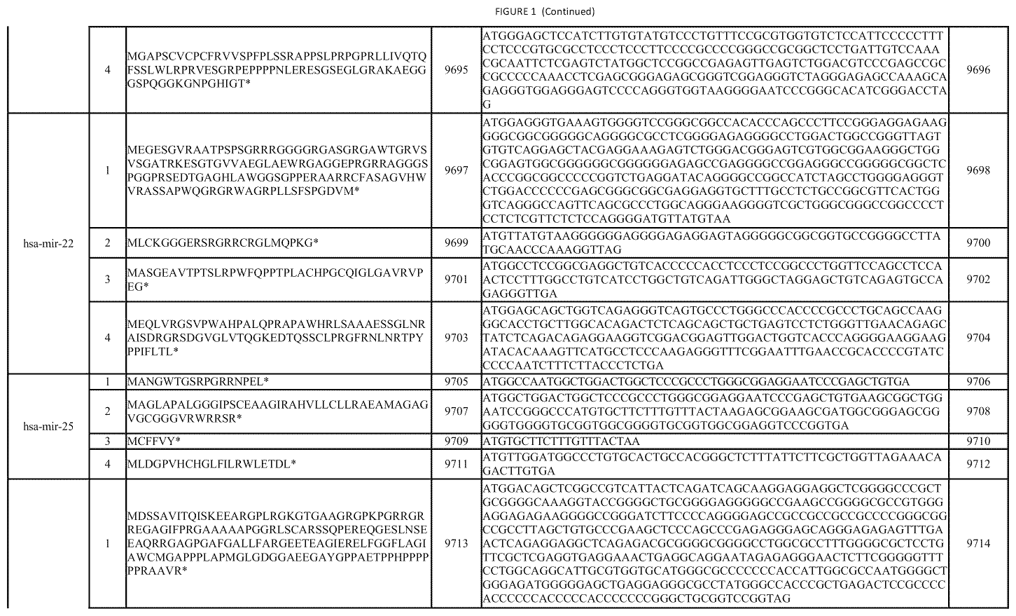

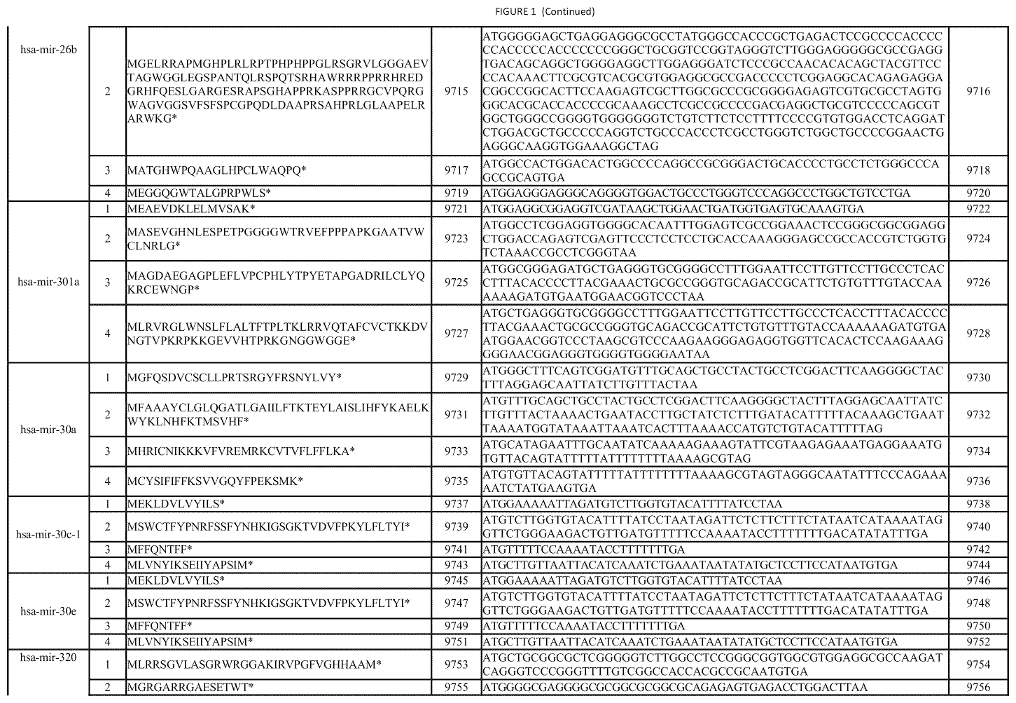

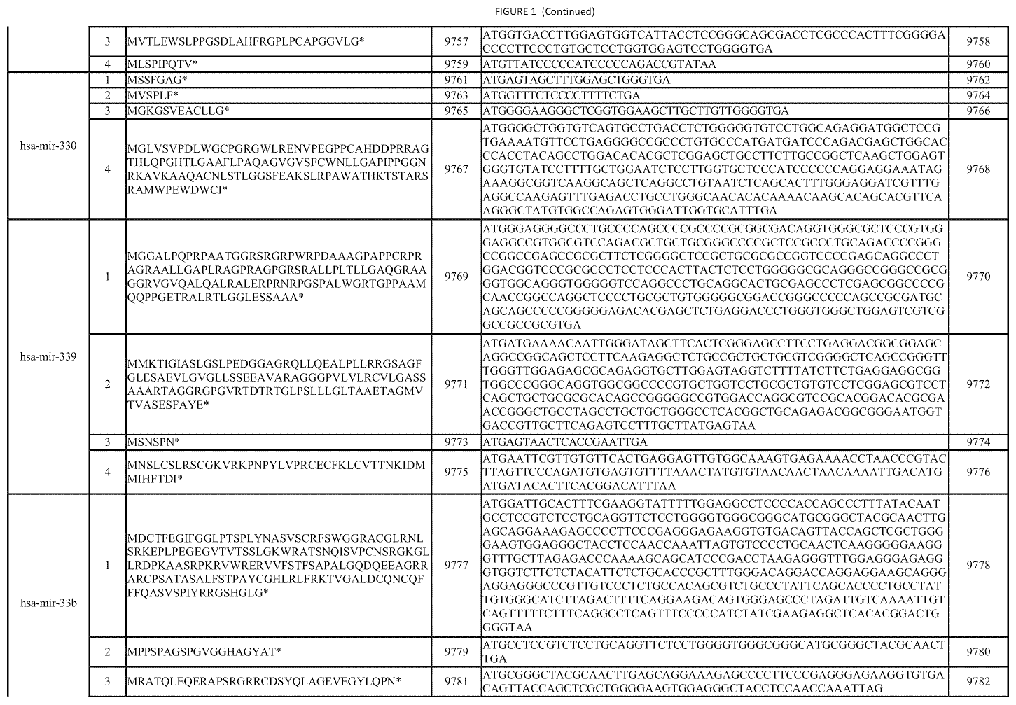

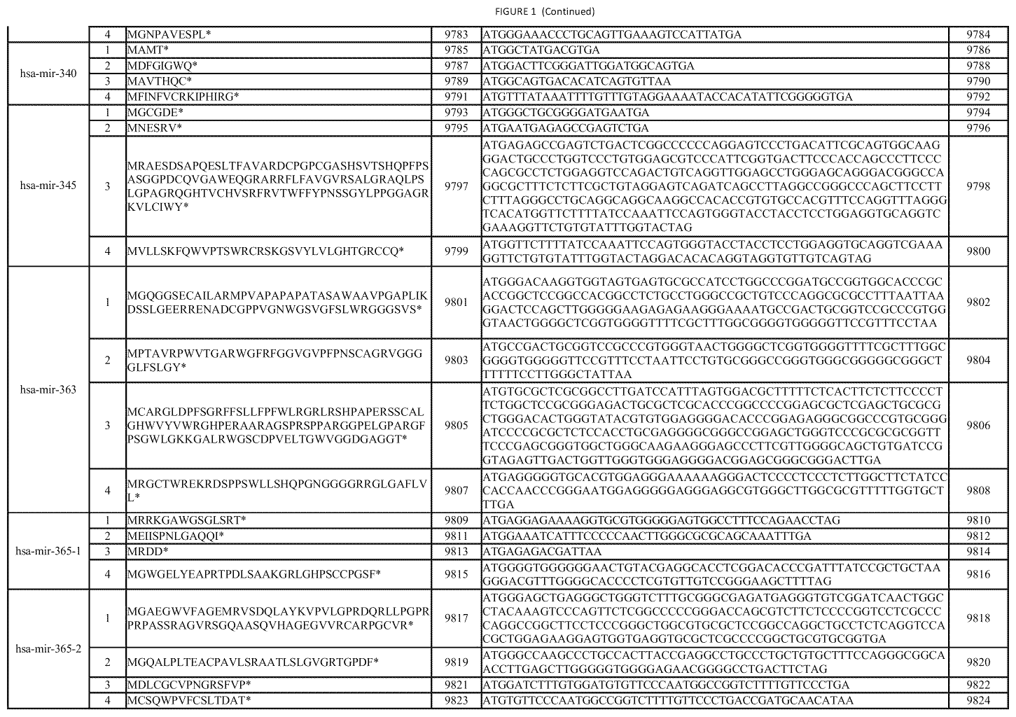

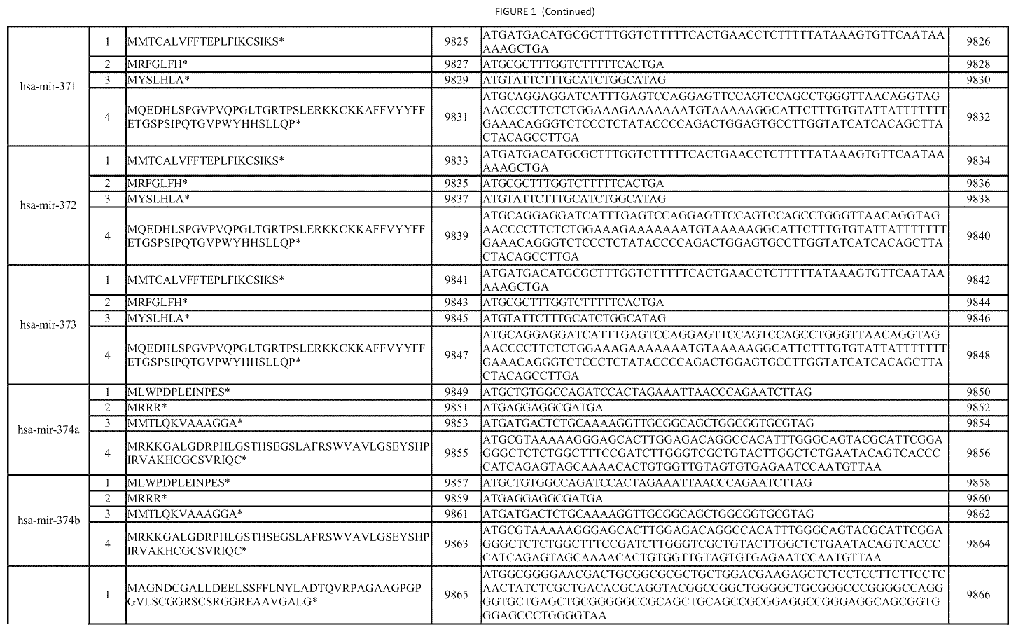

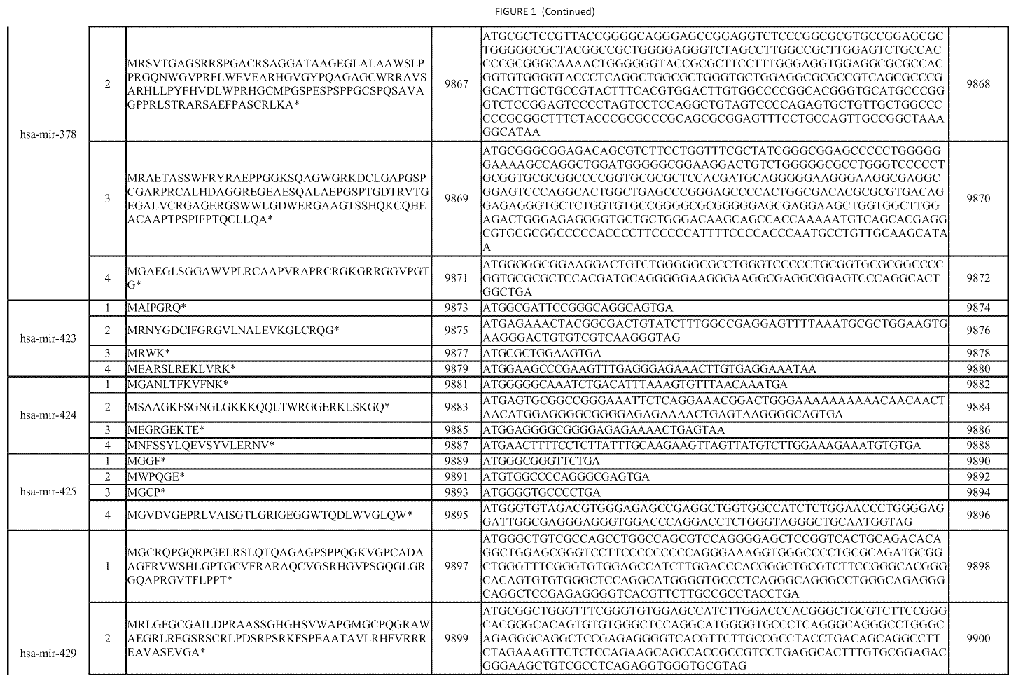

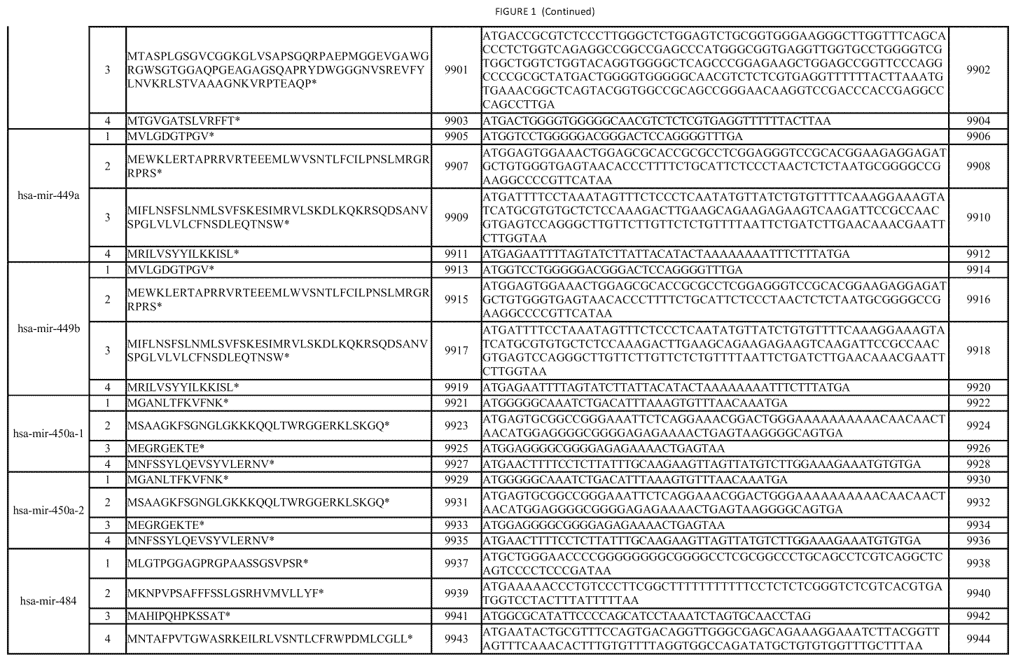

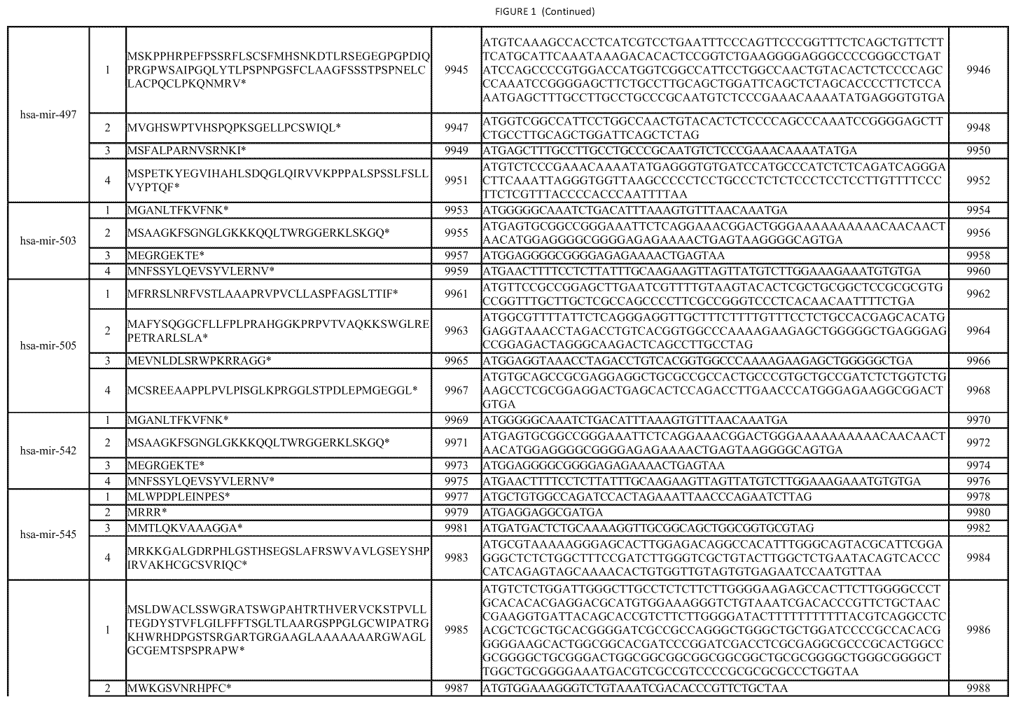

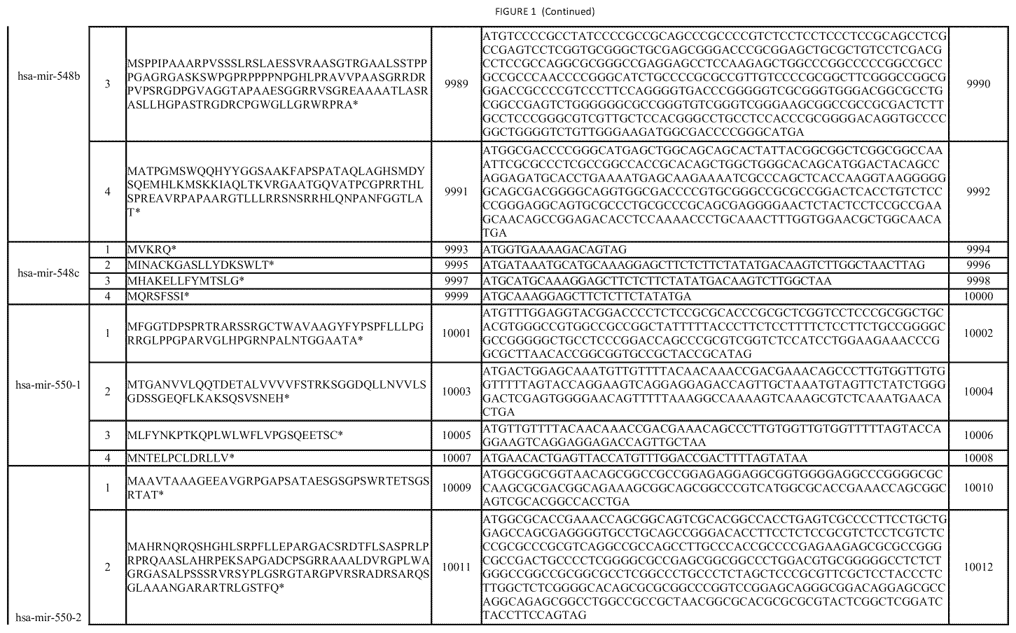

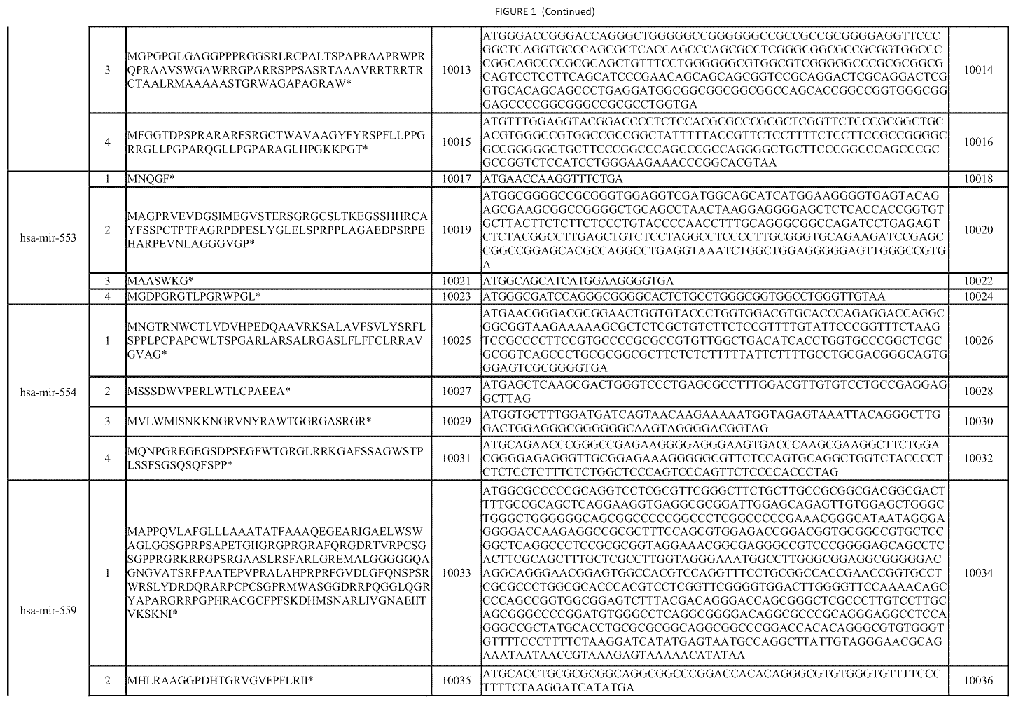

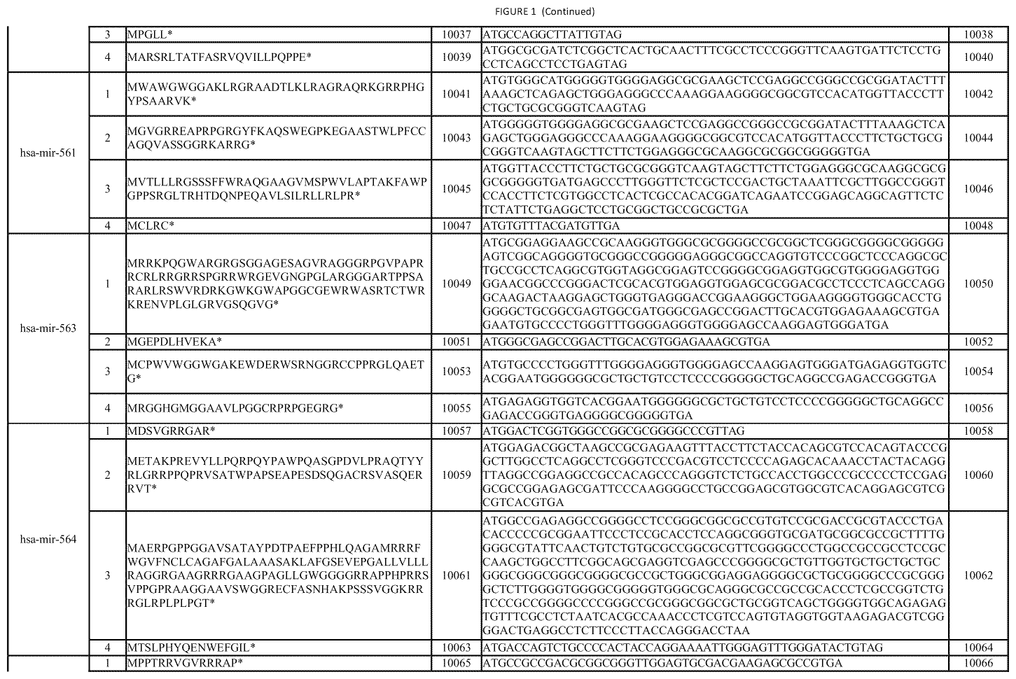

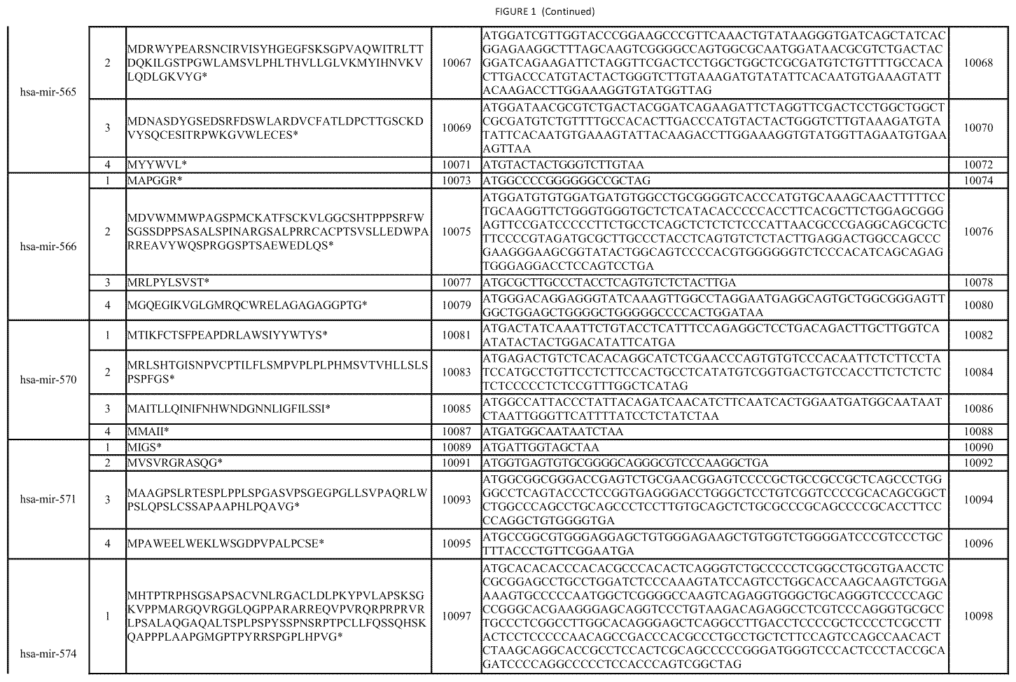

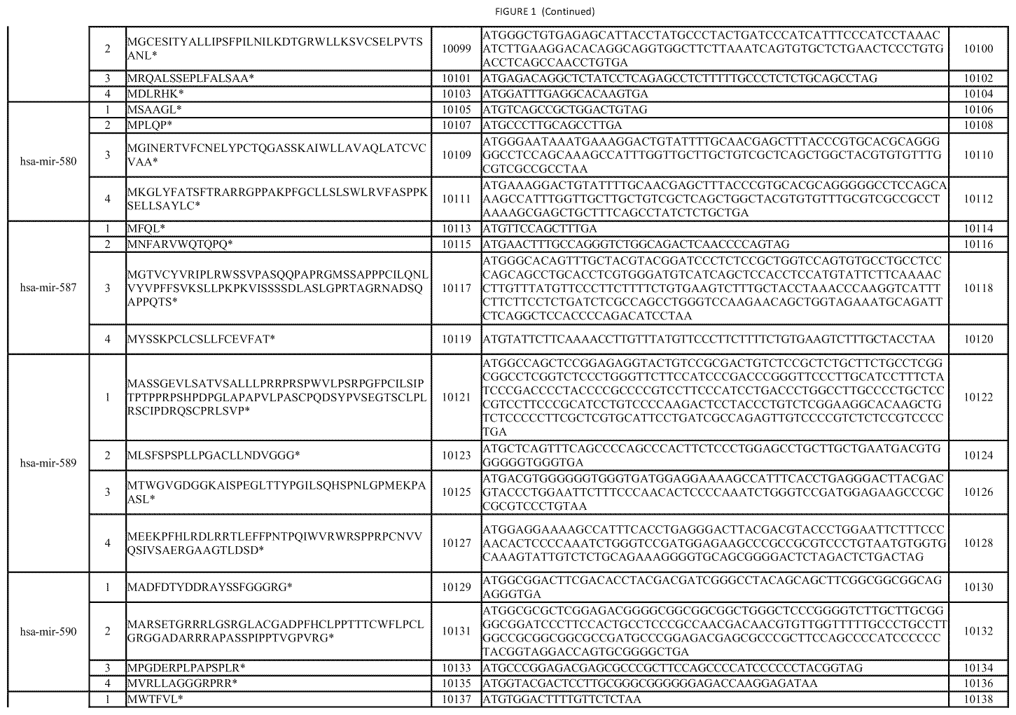

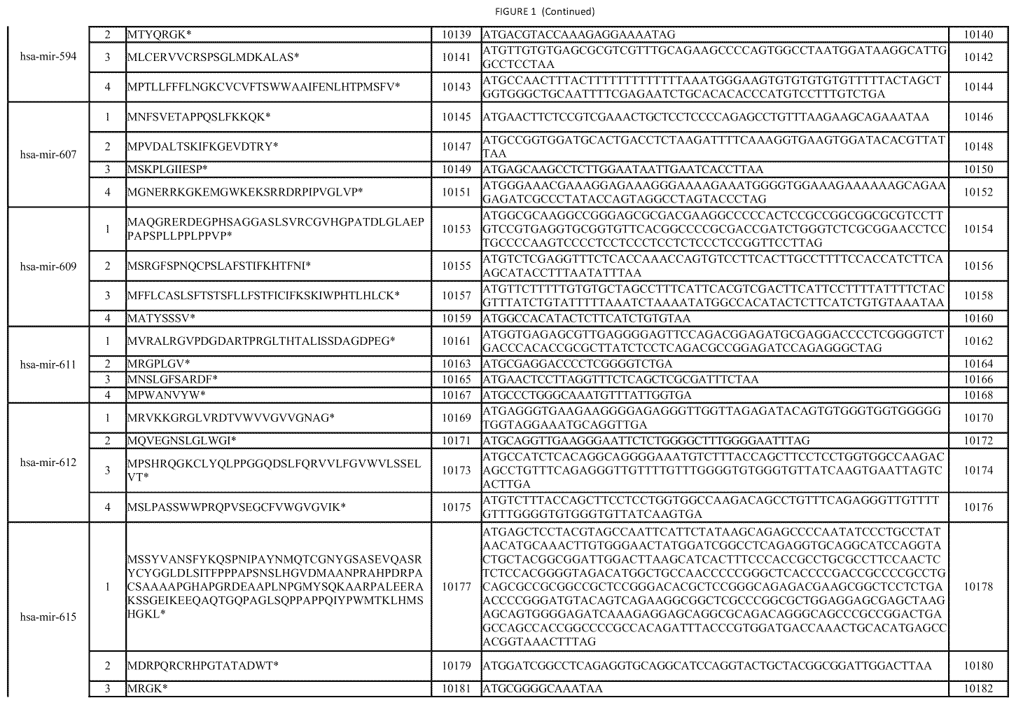

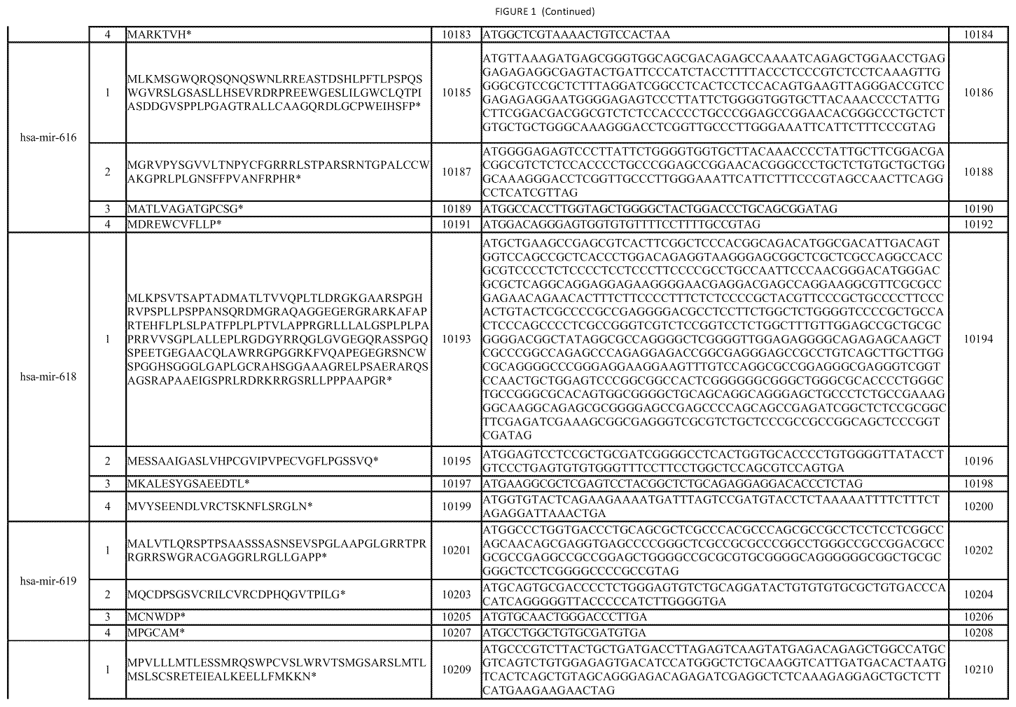

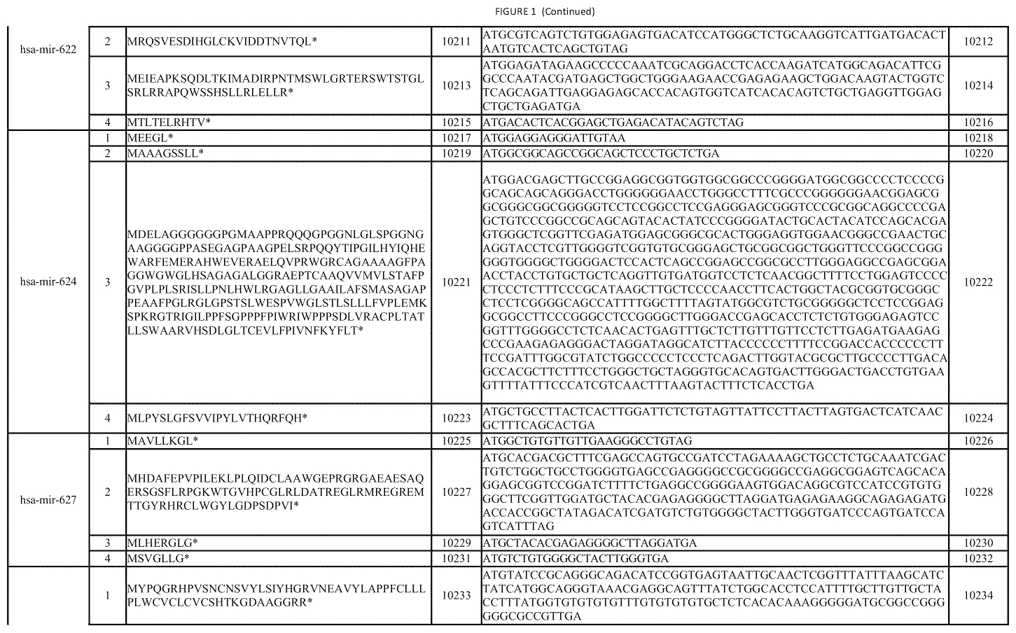

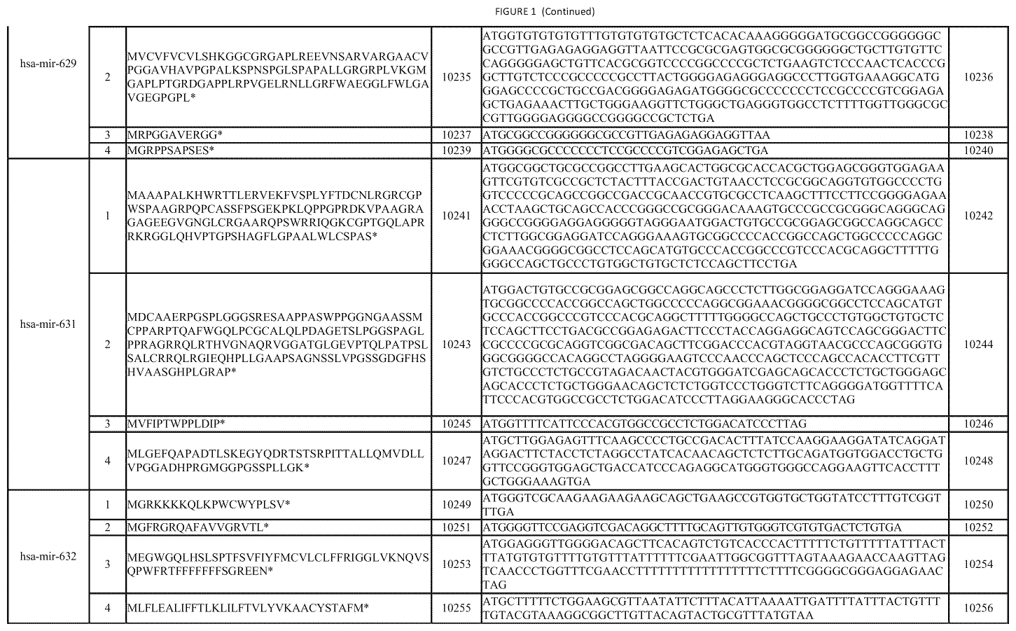

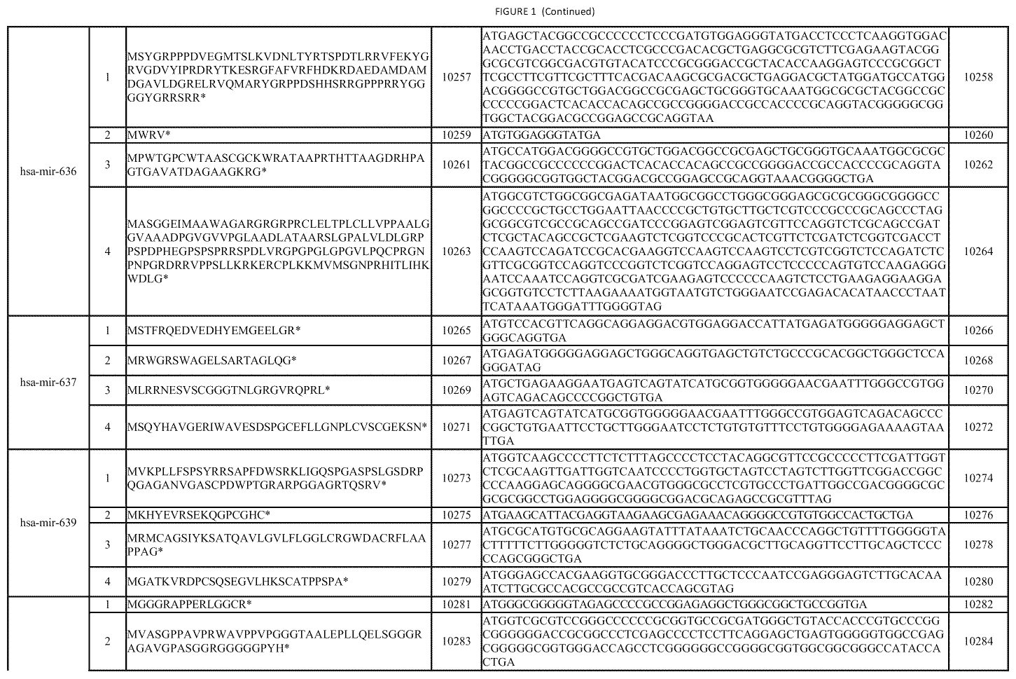

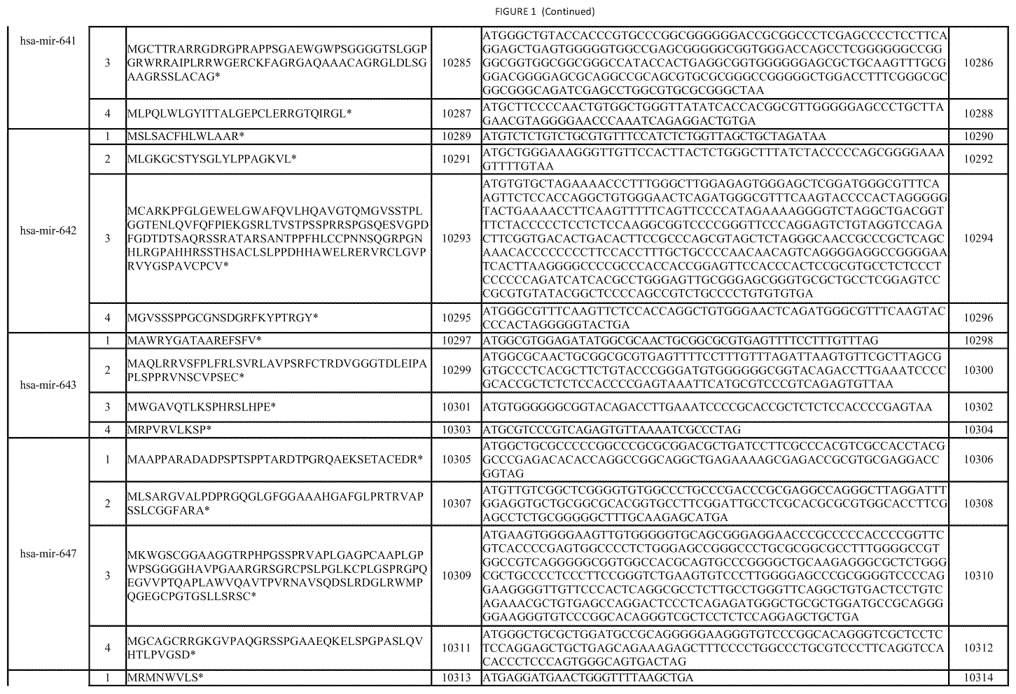

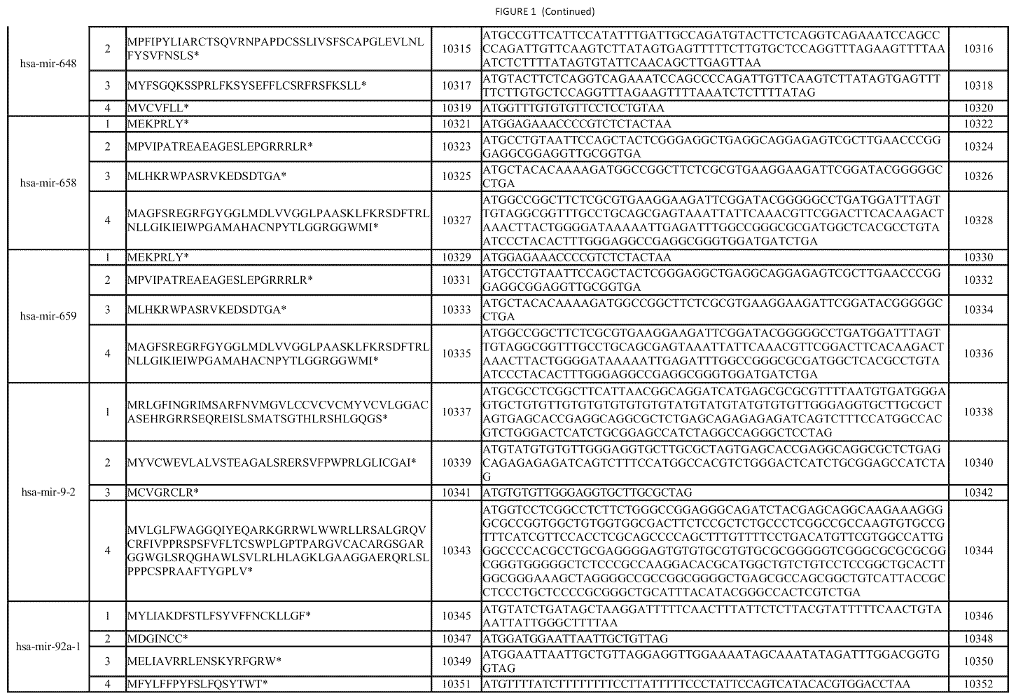

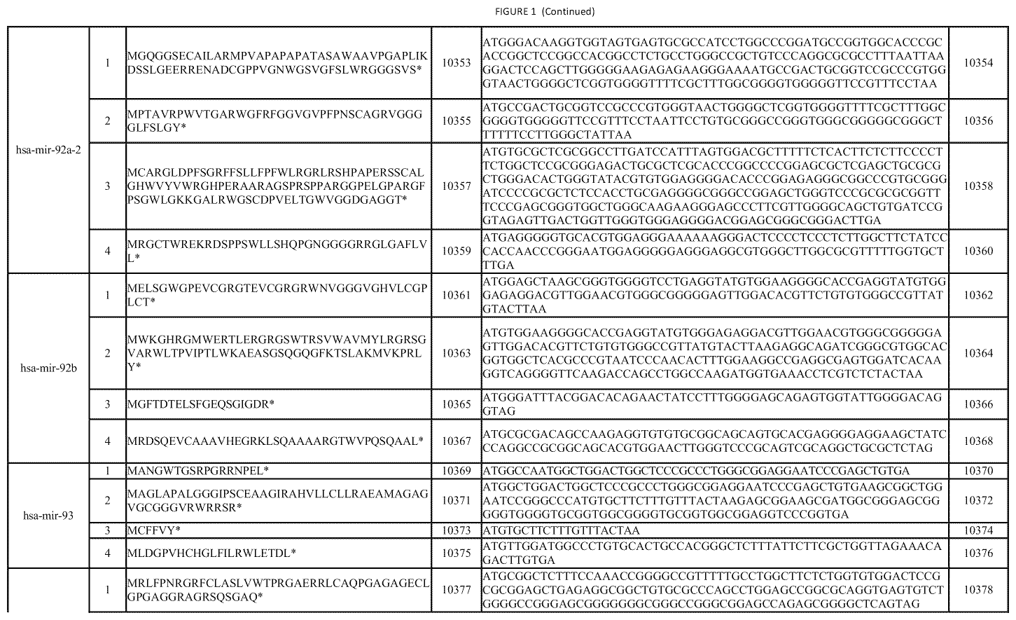

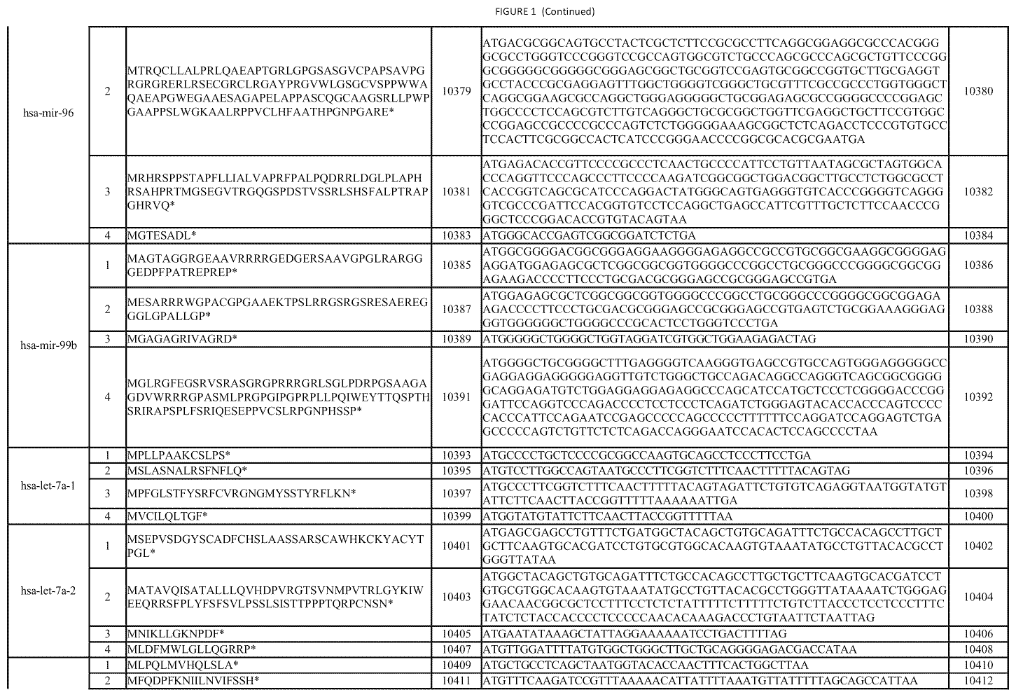

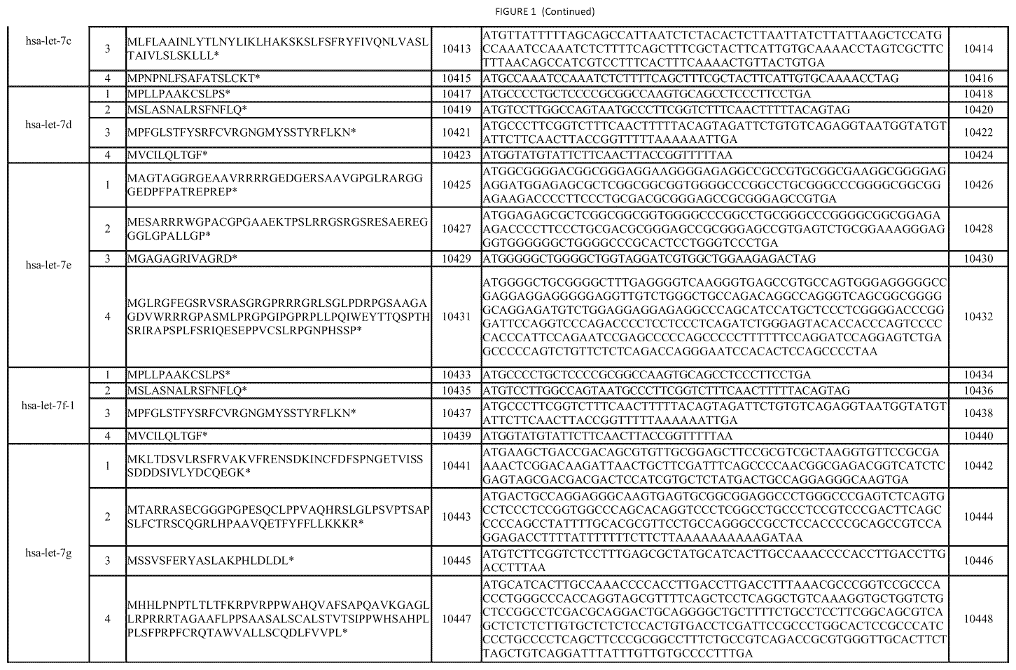

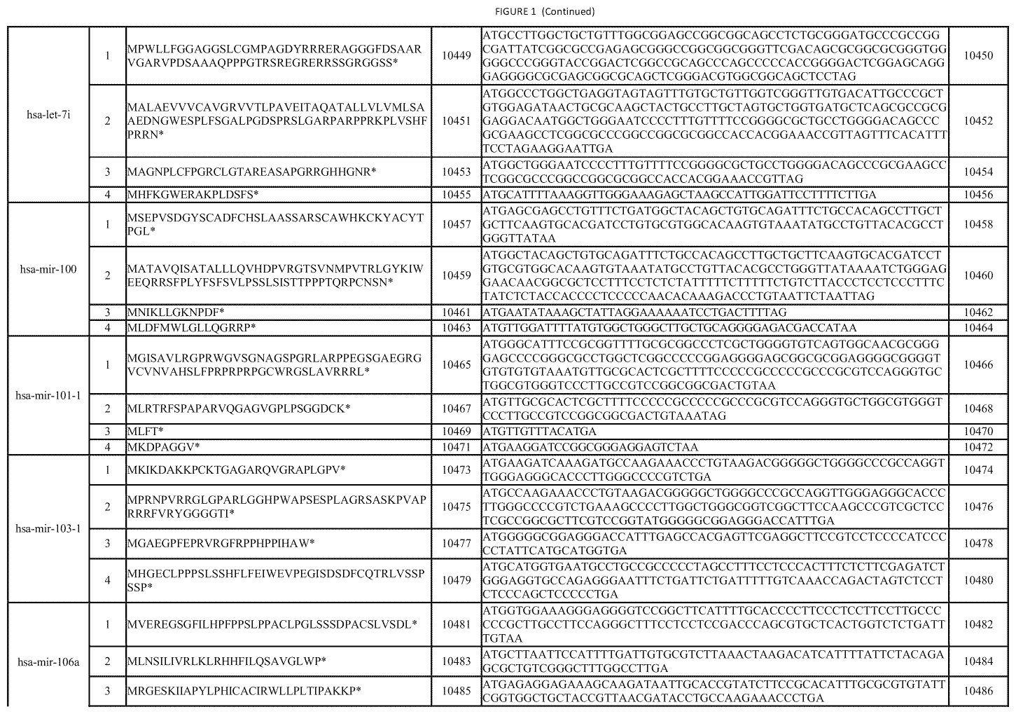

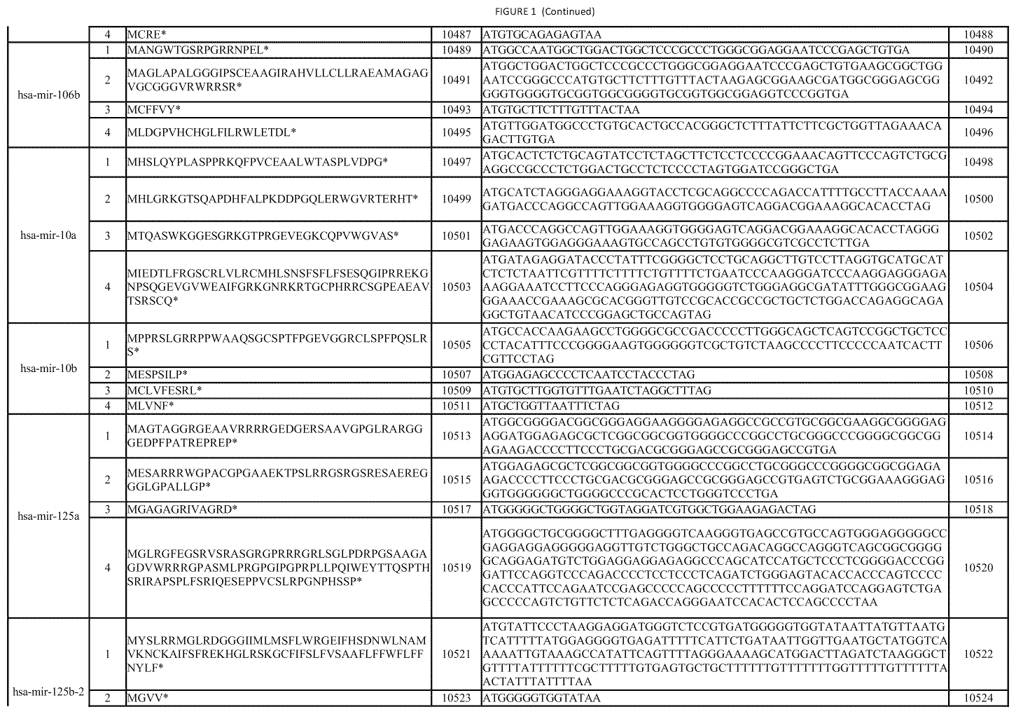

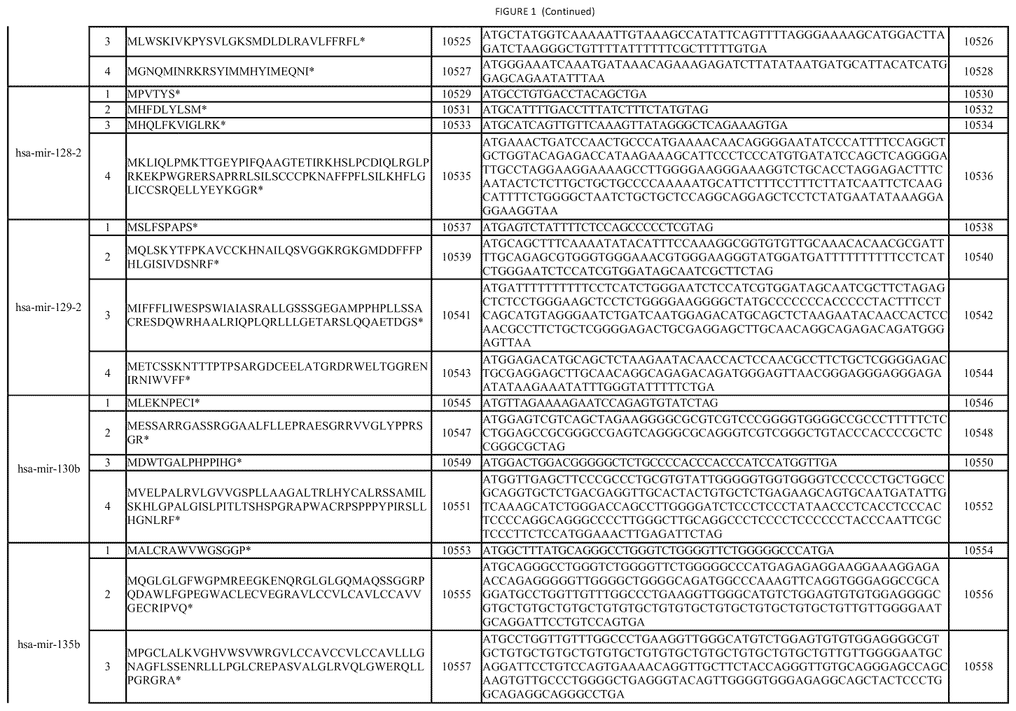

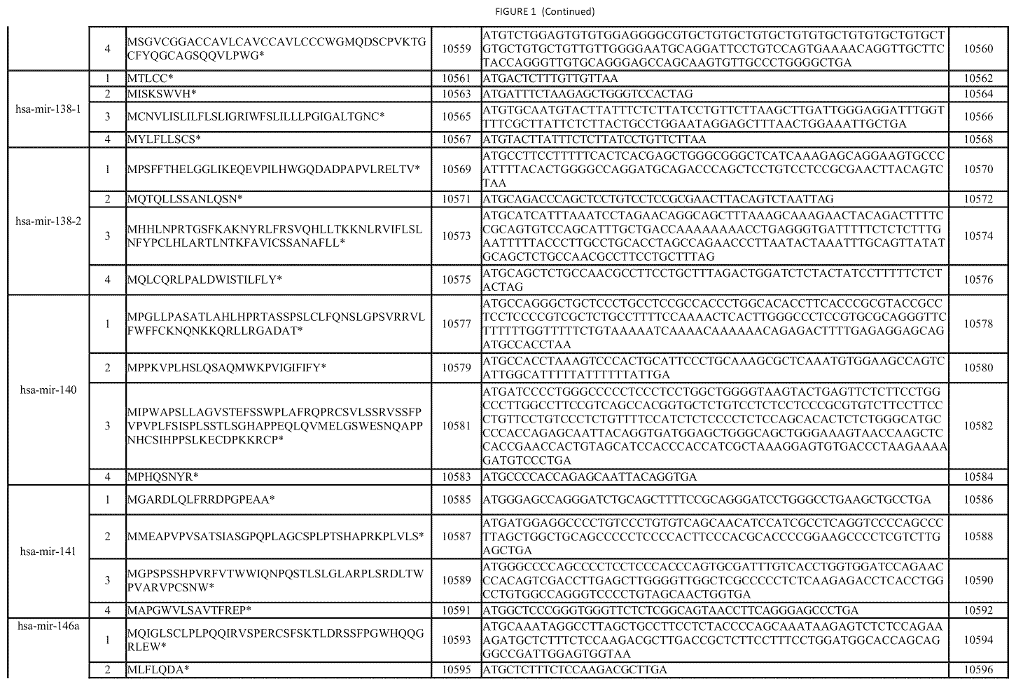

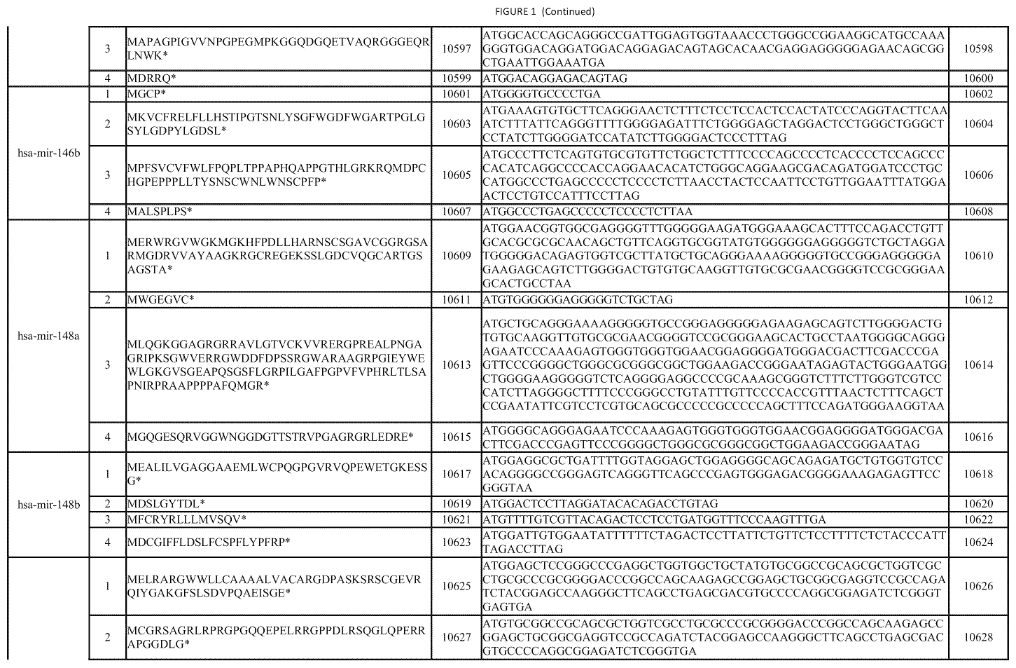

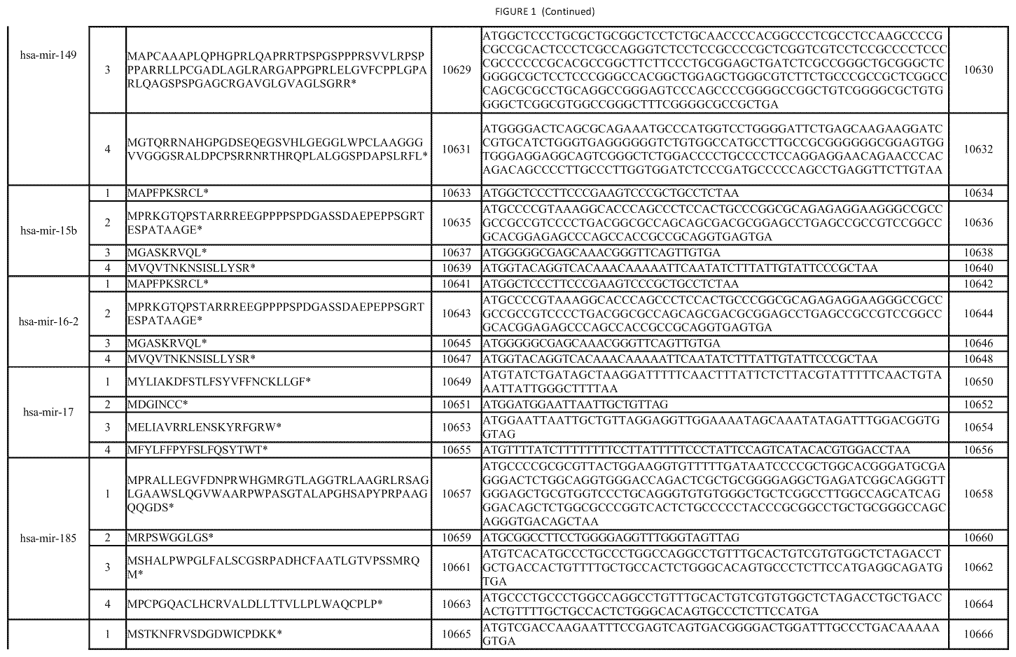

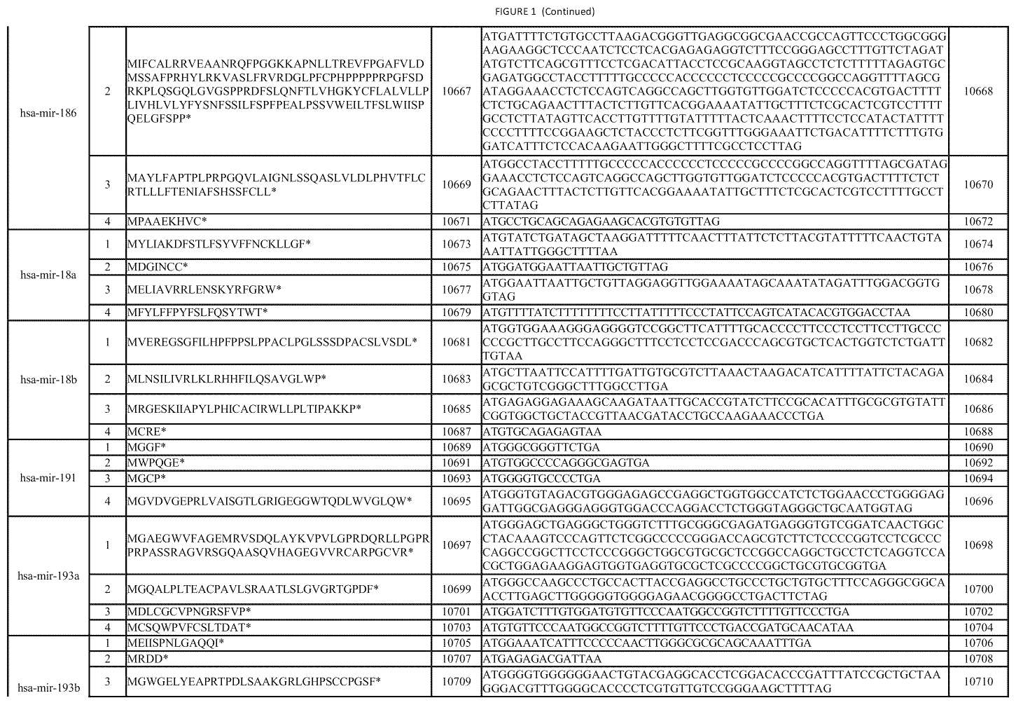

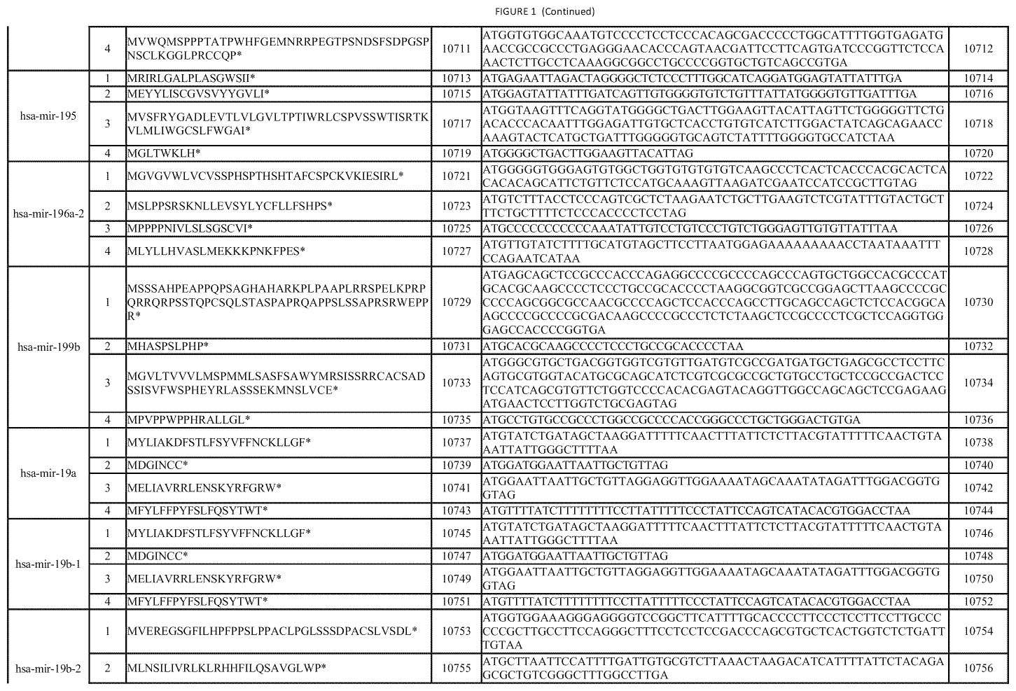

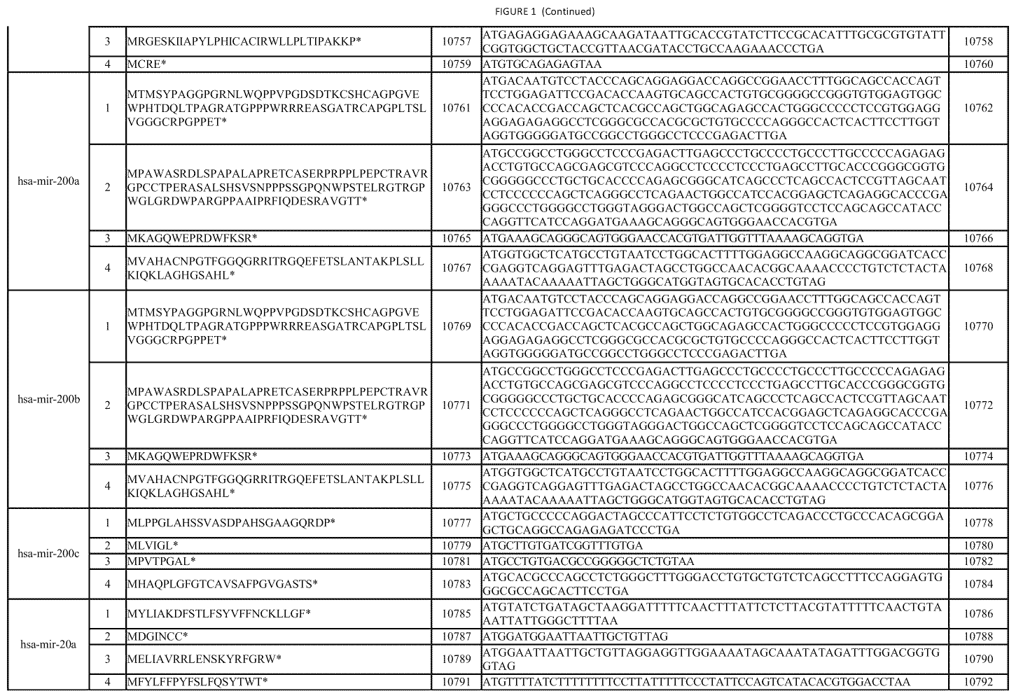

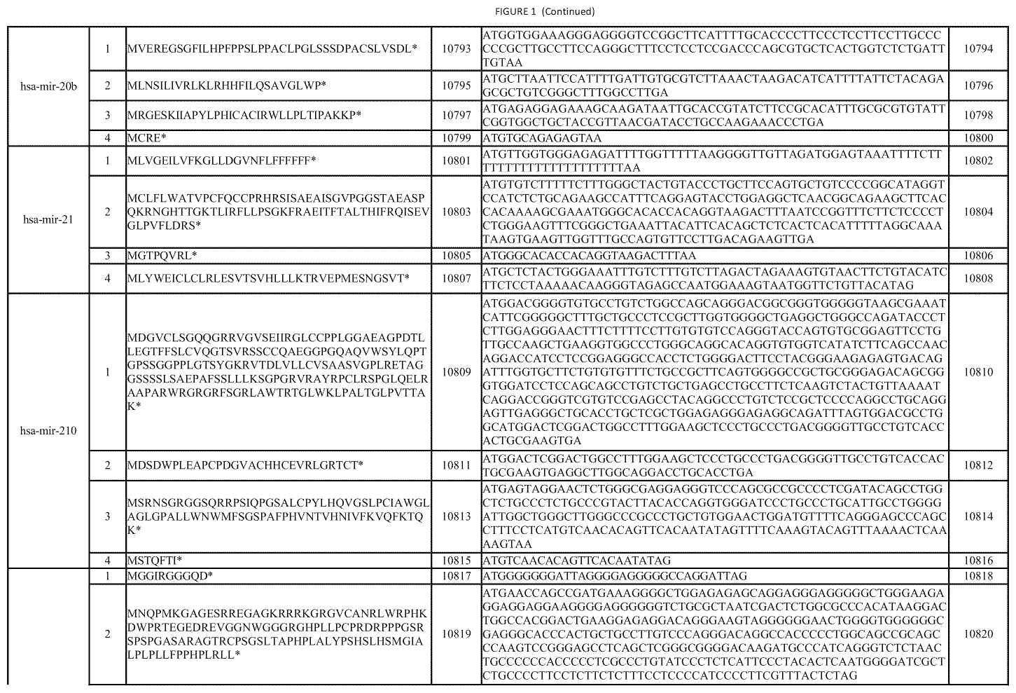

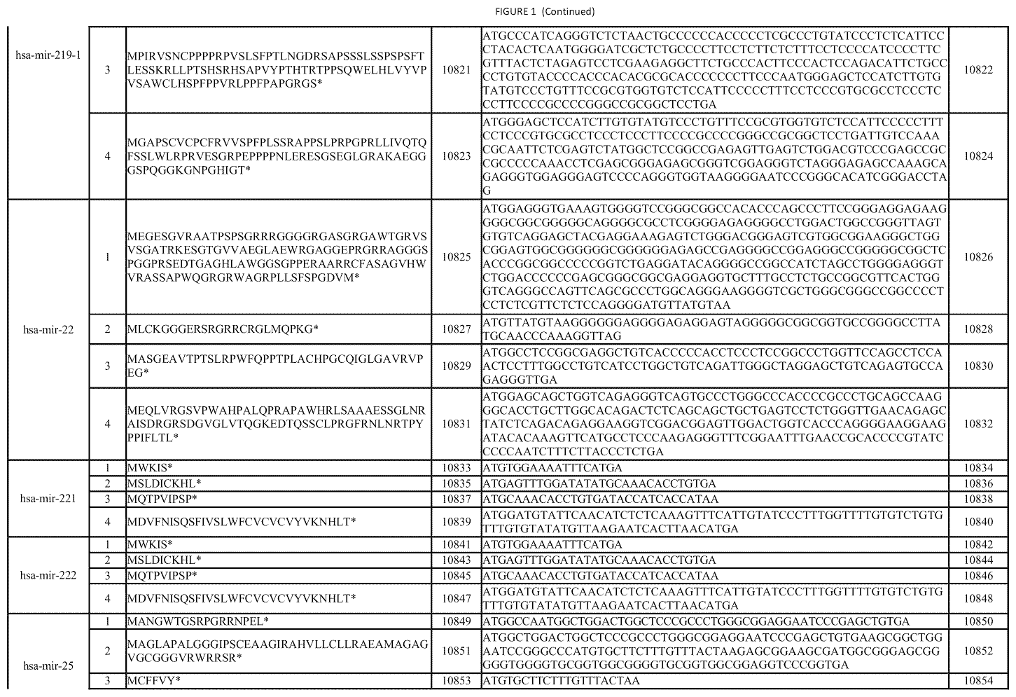

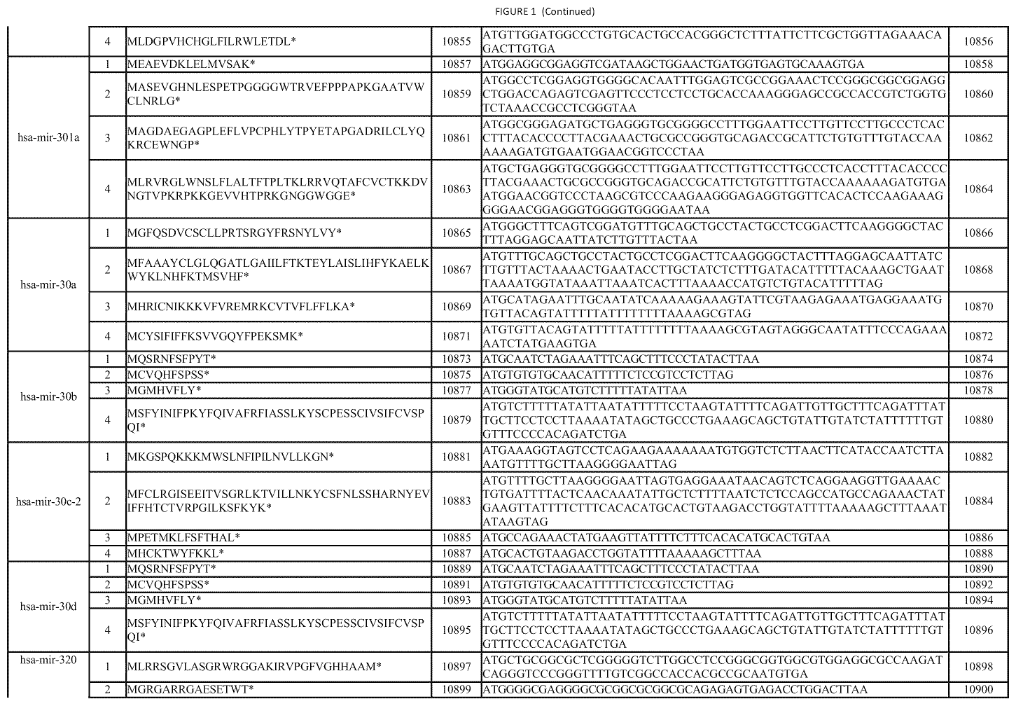

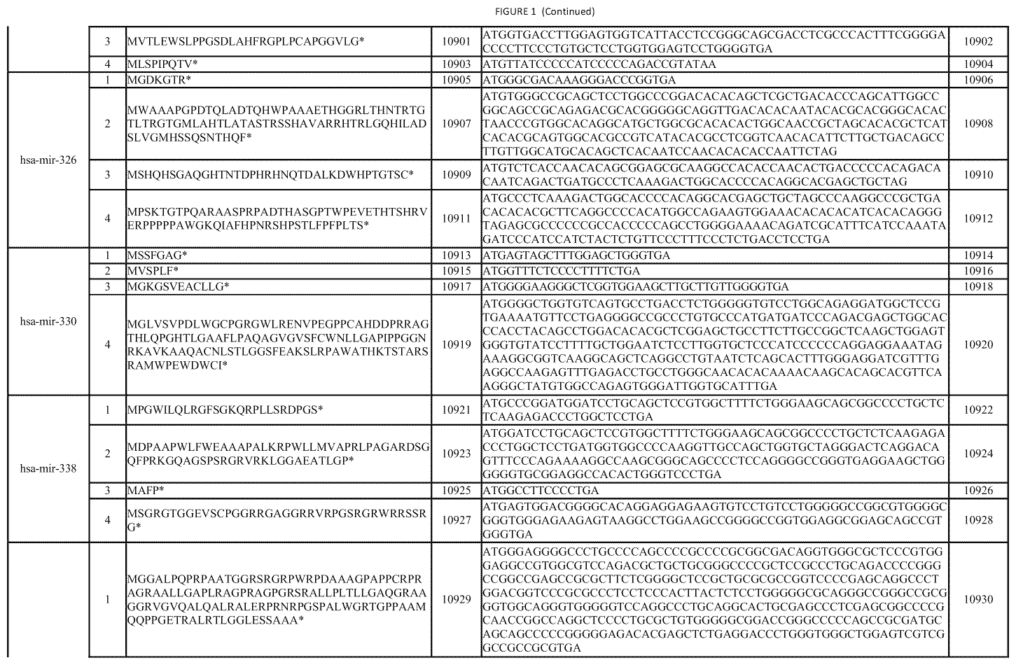

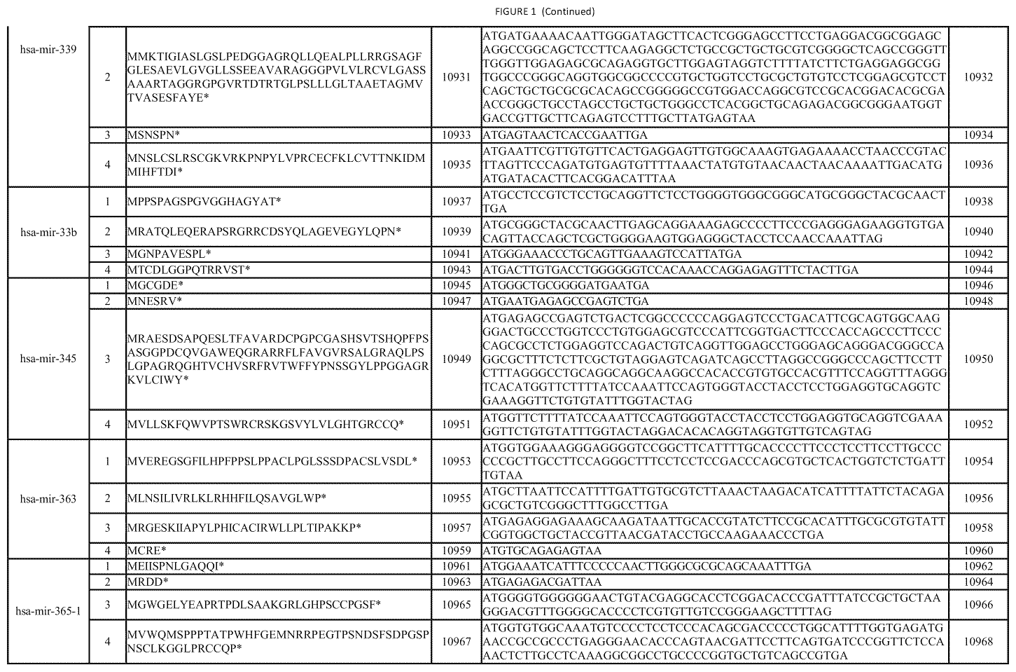

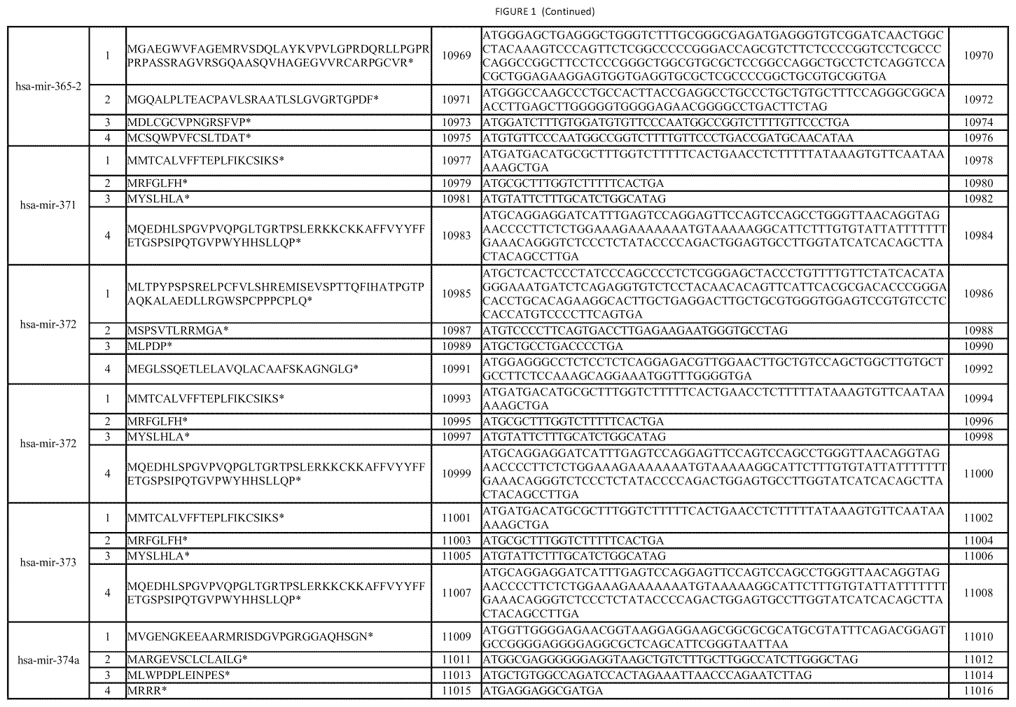

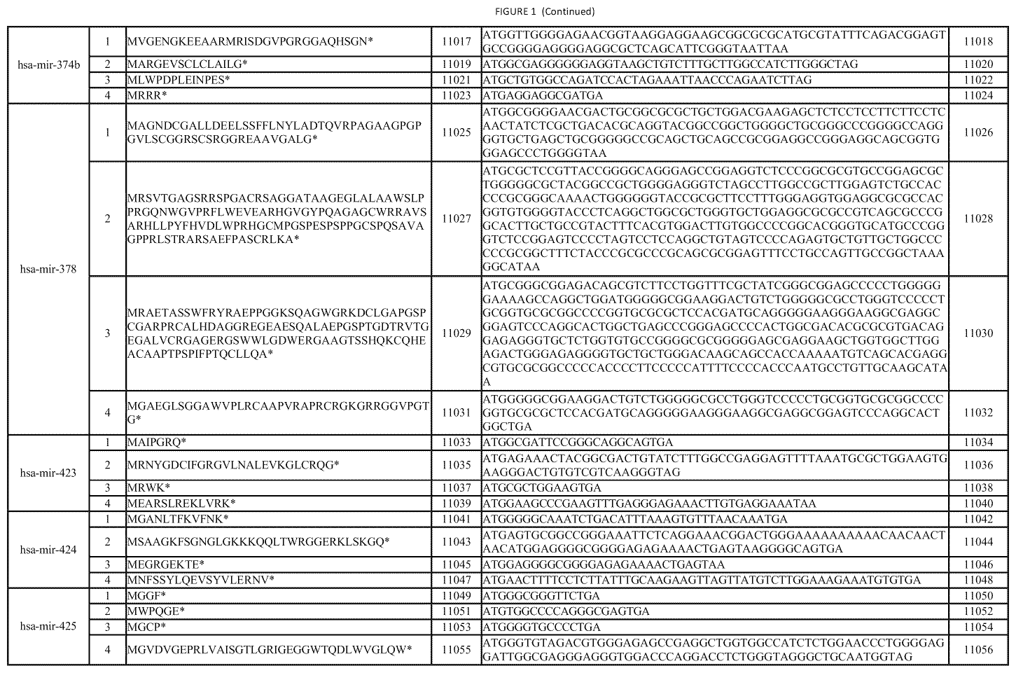

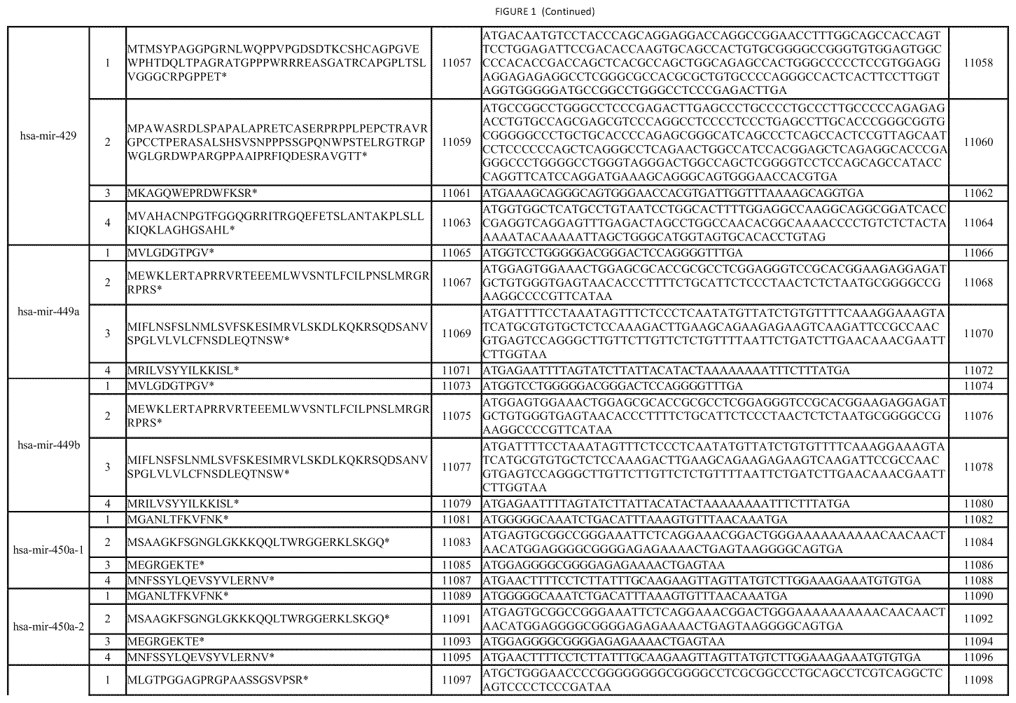

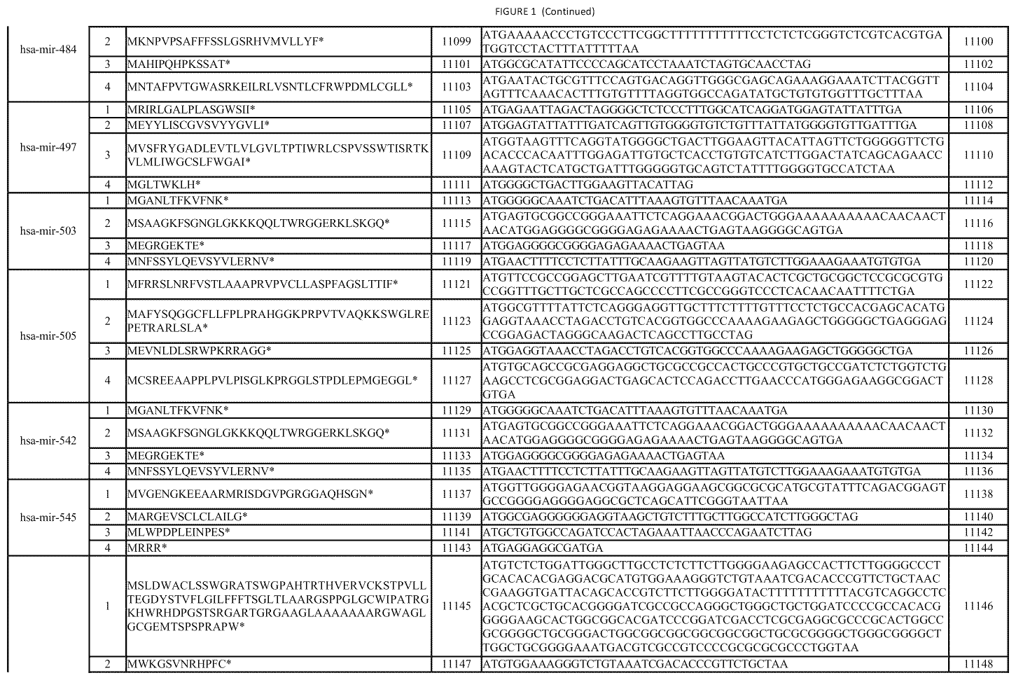

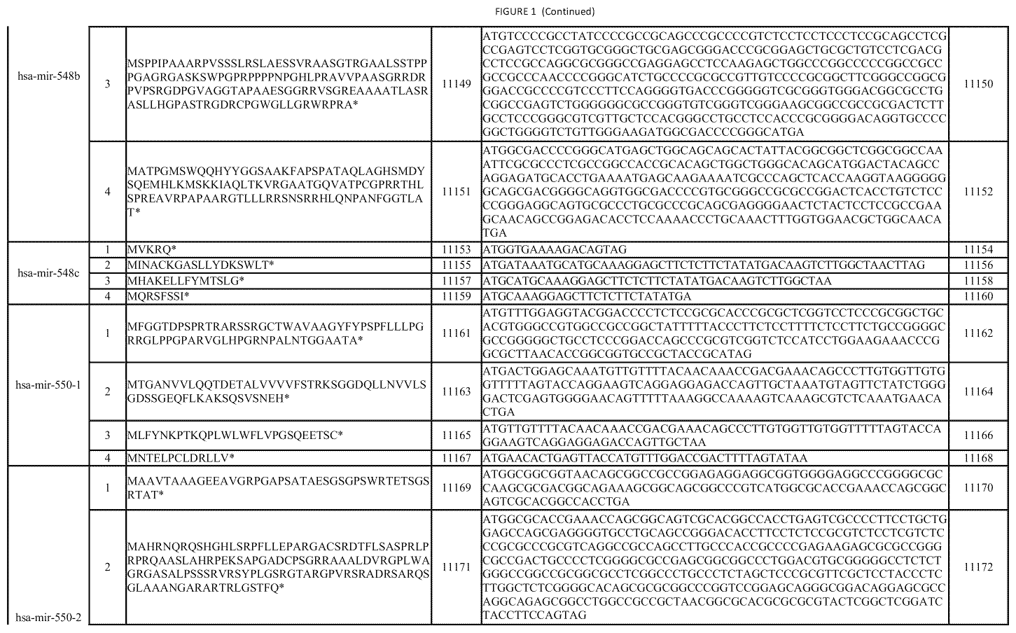

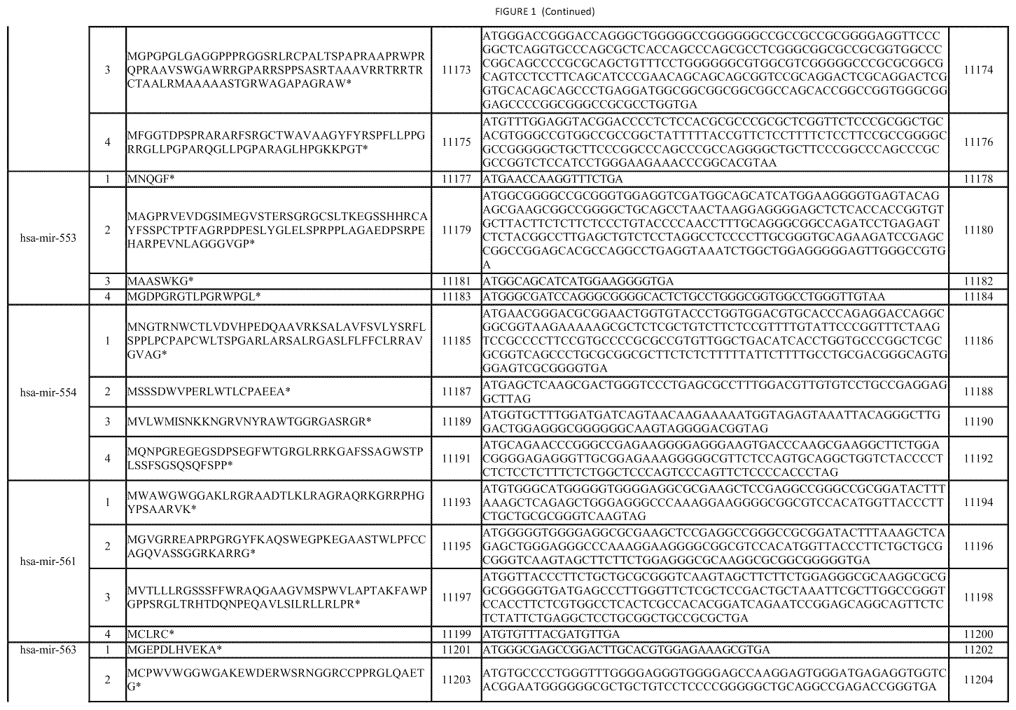

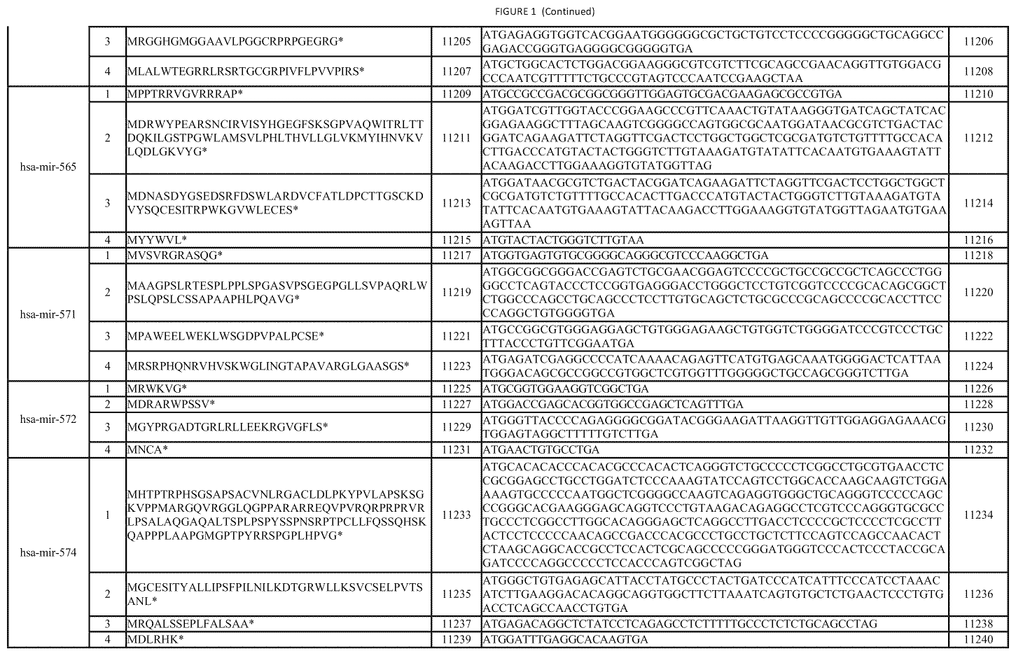

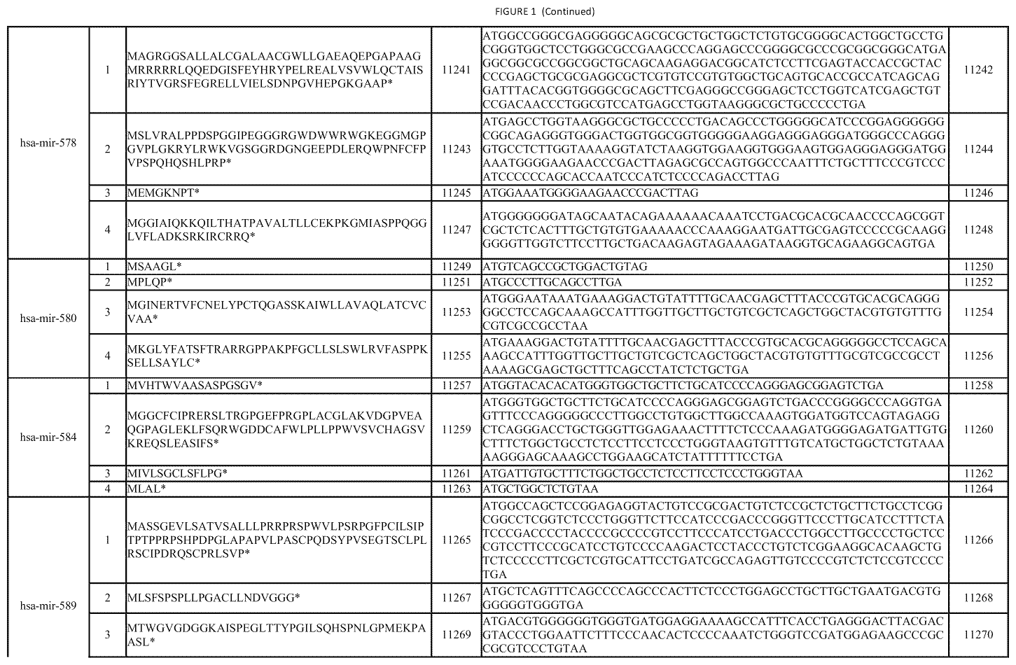

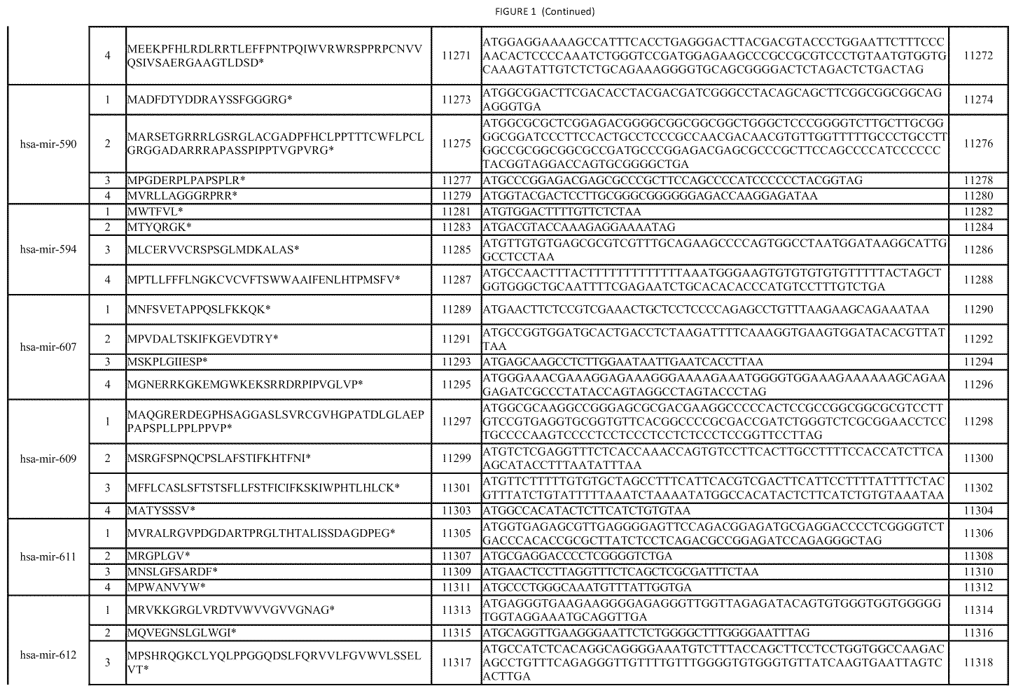

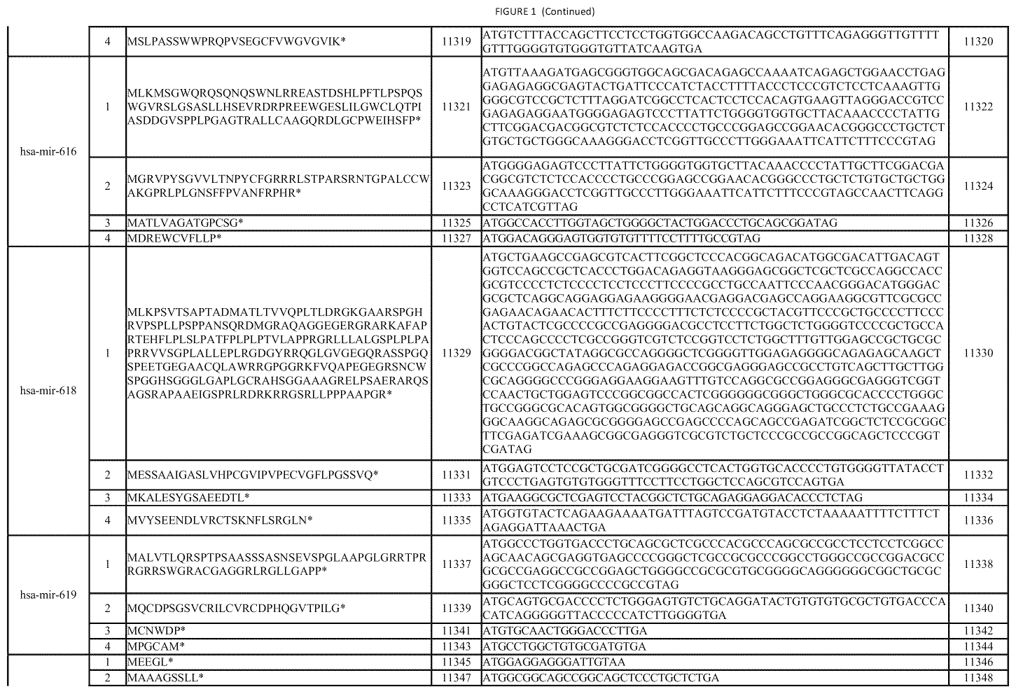

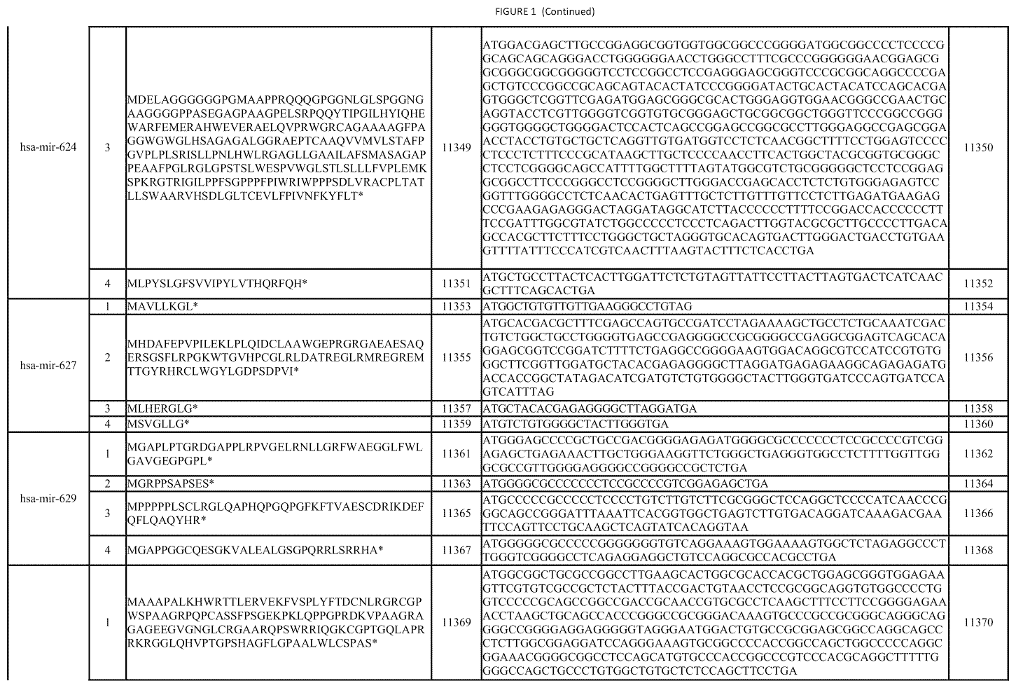

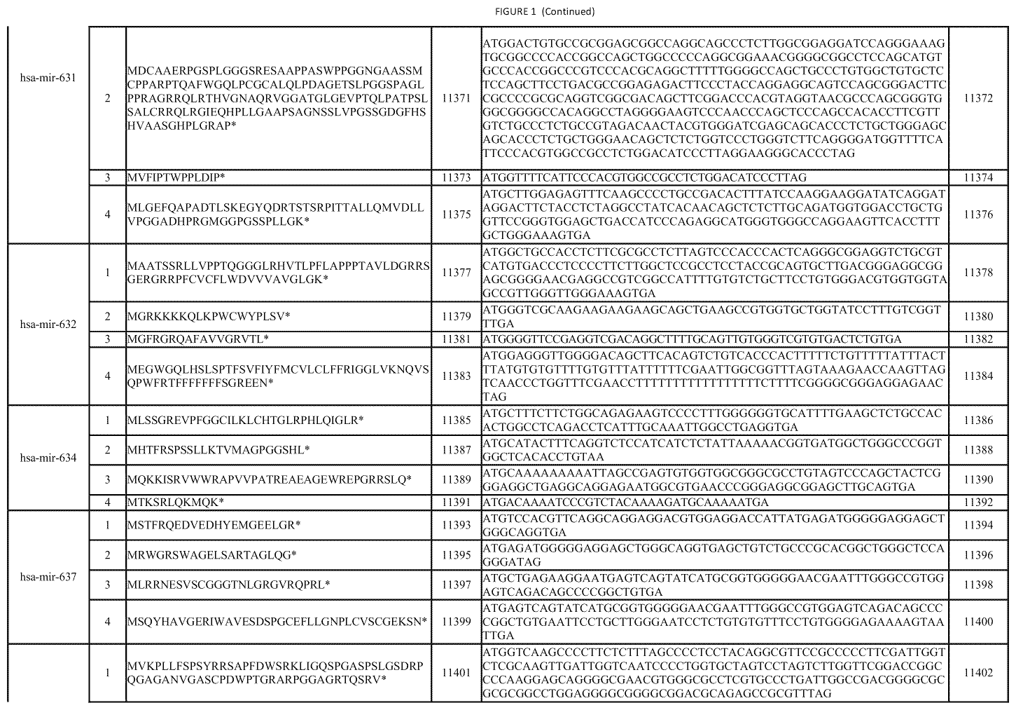

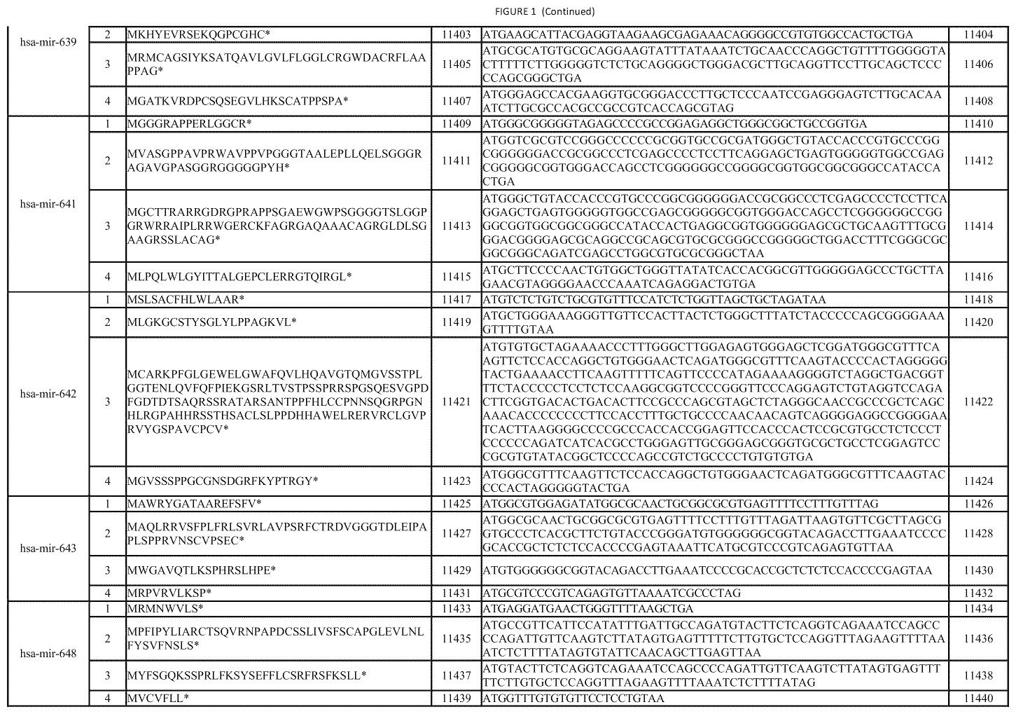

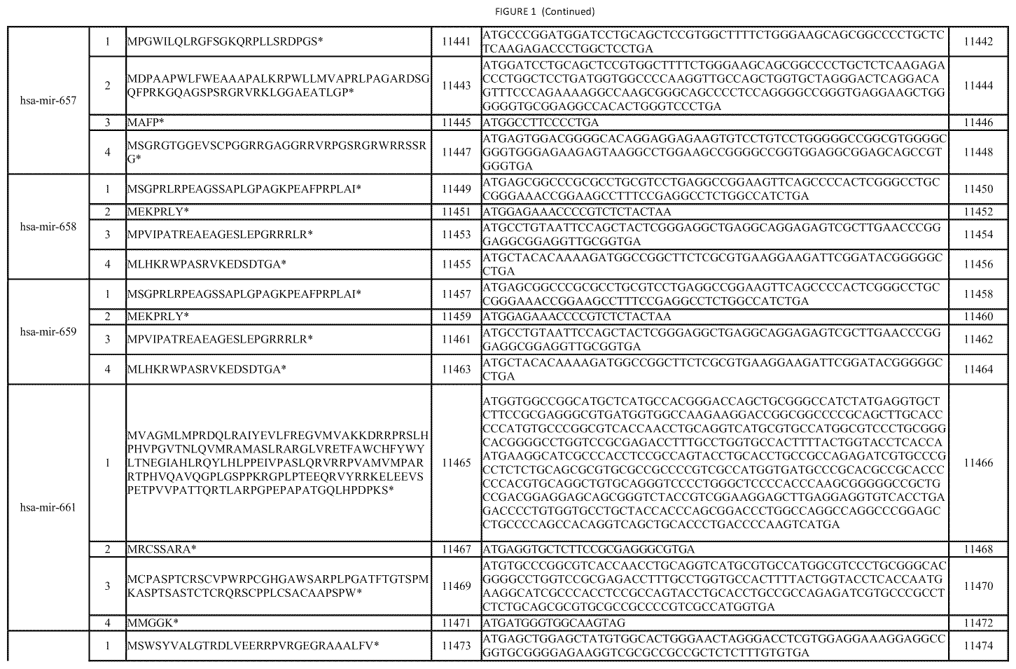

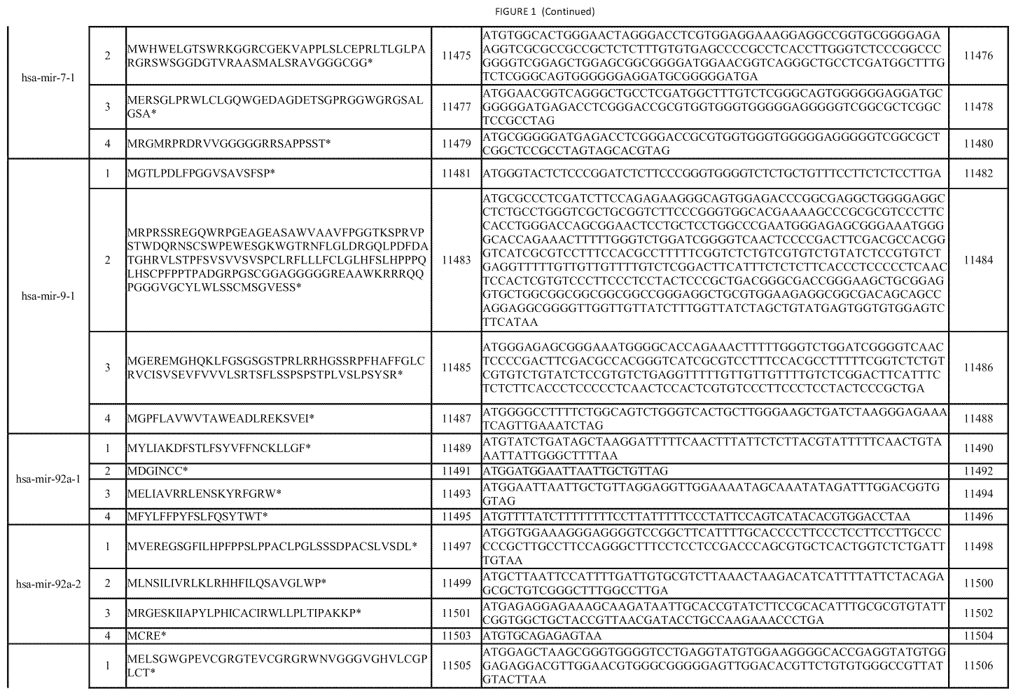

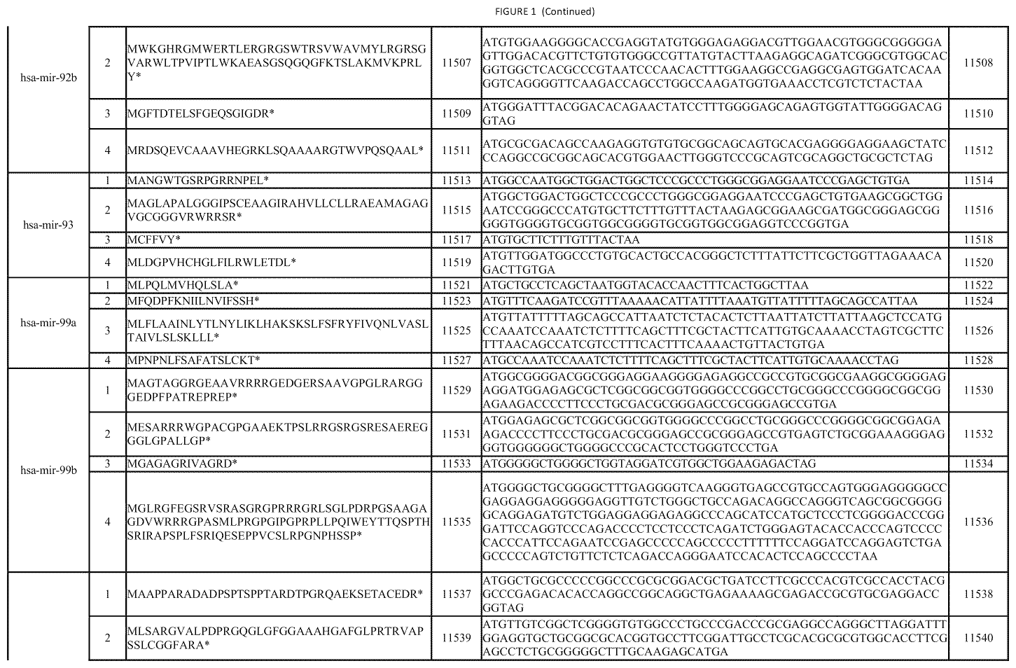

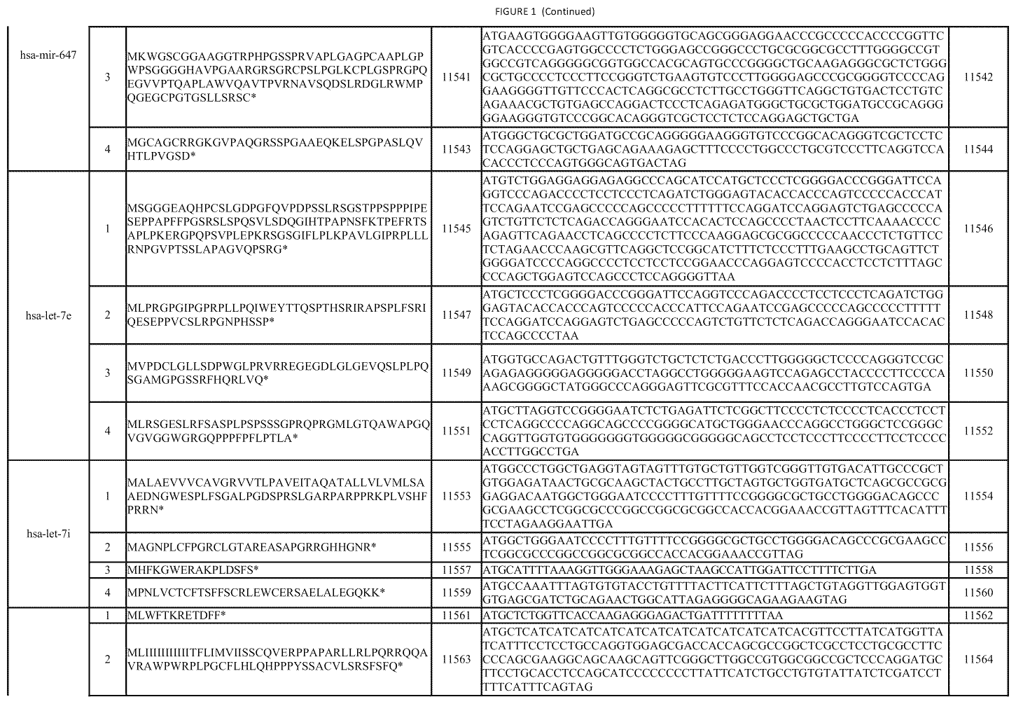

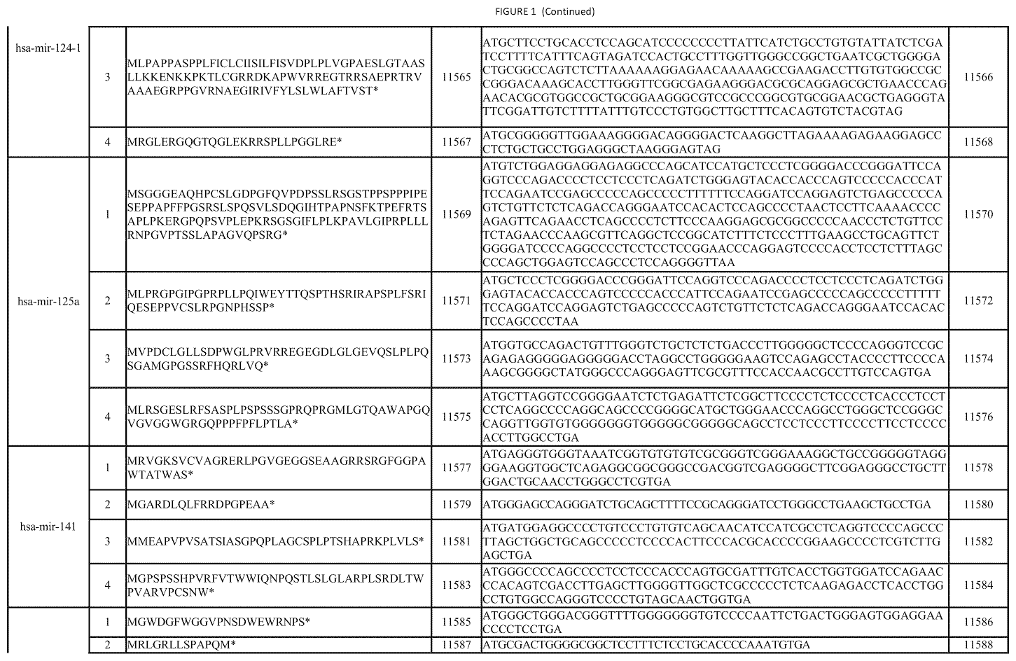

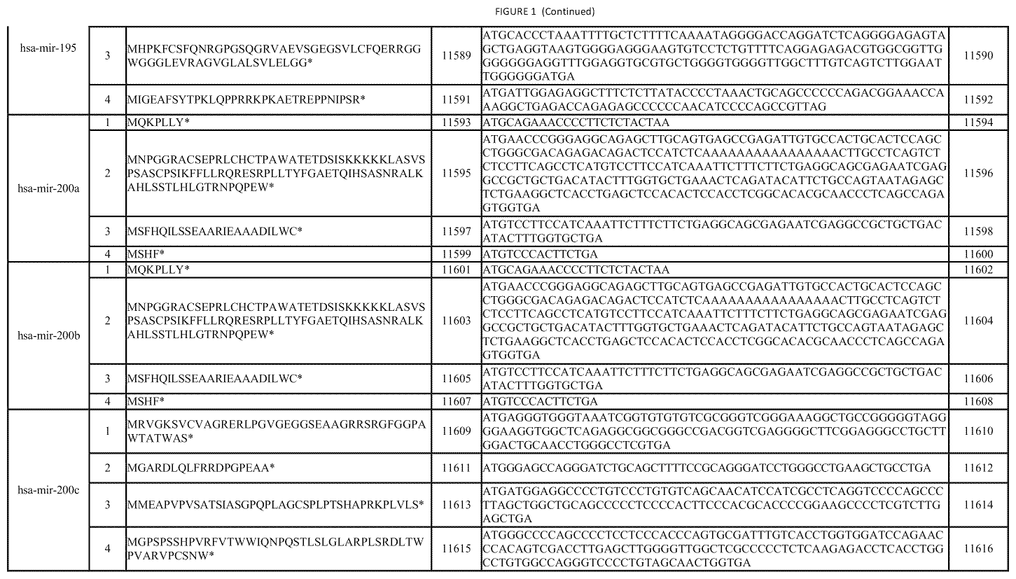

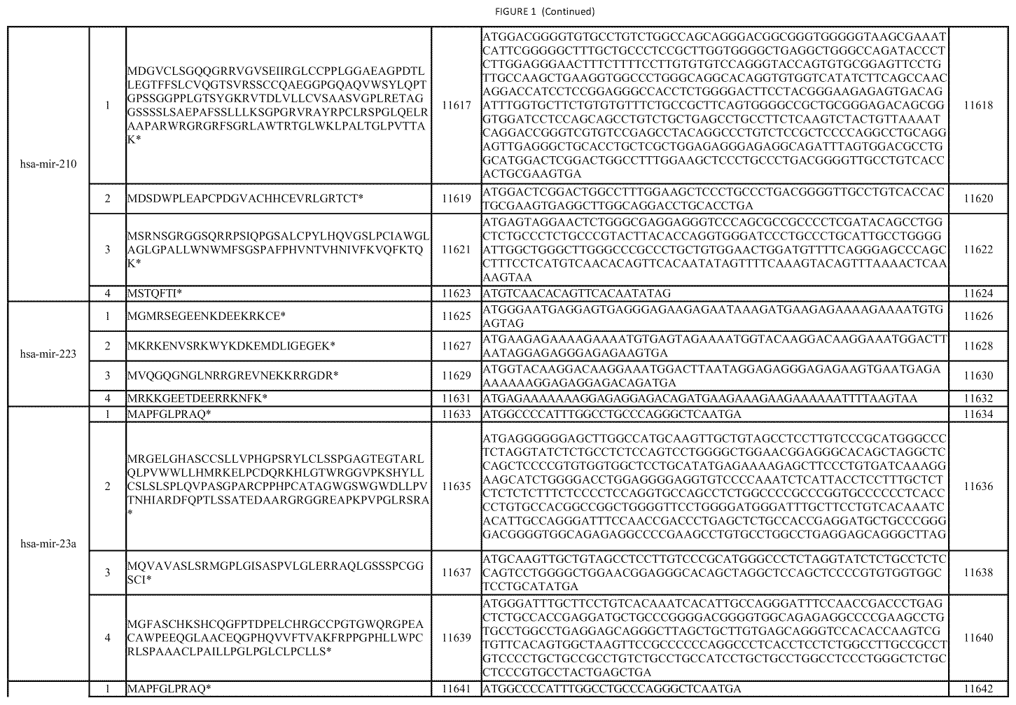

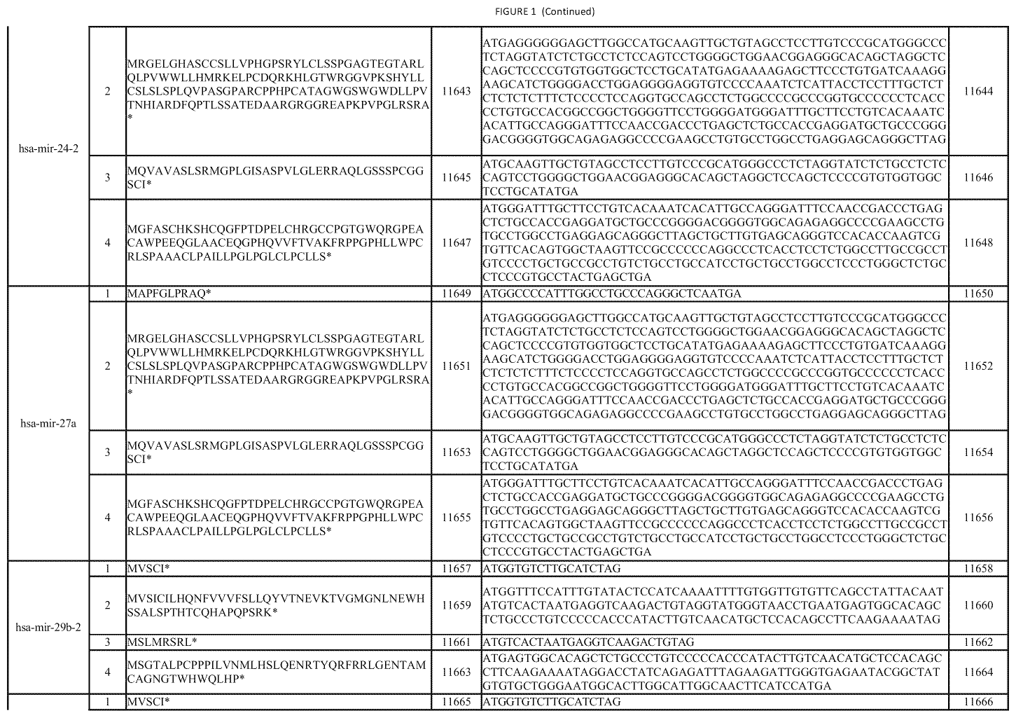

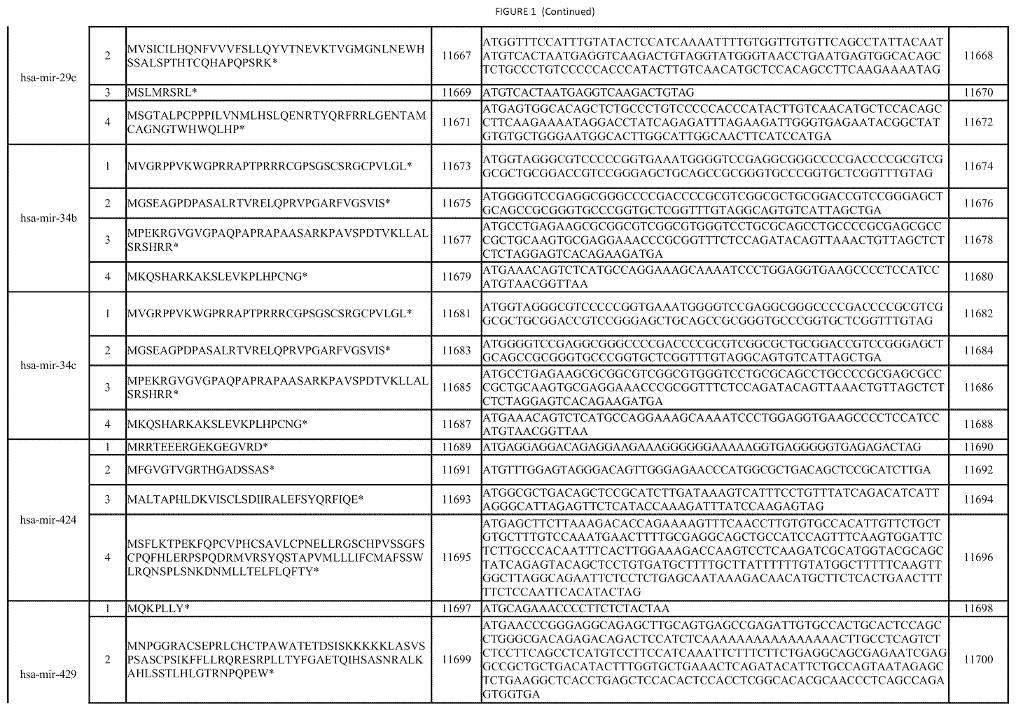

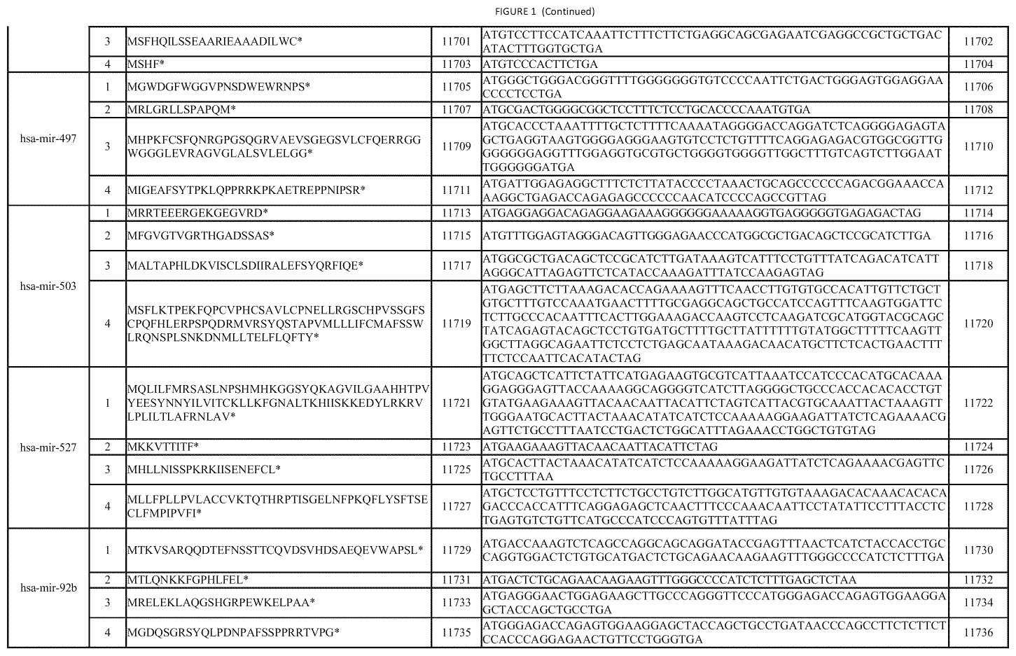

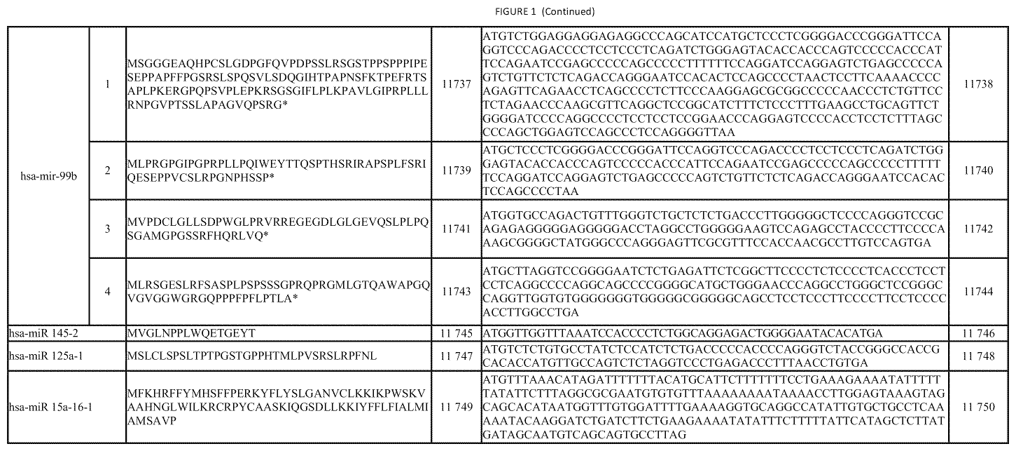

The list of all miPEPs, and corresponding miORPs, is presented in FIG. 1 and is incorporated in this application.

In a particular embodiment, the pharmaceutical compositions comprising the miPEPs are in the form of a unit dose comprising from 10⁻⁴ M to 10⁻¹⁰ M of miPEP.

In a particular embodiment, the compositions according to the invention can be administered in one or more times.

In a particular embodiment, the pharmaceutical compositions are suitable for oral, ocular, nasal, parenteral (intravenous, intraarterial, subcutaneous, intradermal, intramuscular), rectal, vaginal, topical or auricular administration.

In a particular embodiment, the miPEP can be vectorized or encapsulated.

In the invention, the miPEP, or the fragment of said miPEP, may be fused or linked to one or more molecules facilitating the entry of the miPEP, or miPEP fragment, into the cell.

In a particular embodiment, the invention relates to a pharmaceutical composition as defined above, wherein said miPEP, or said fragment of said miPEP, is fused to a peptide facilitating the entry into the cell of the miPEP, or miPEP fragment.

In a particular embodiment, the invention relates to a pharmaceutical composition as defined above, wherein said miPEP, or said fragment of said miPEP, is fused to the TAT peptide (YGRKKRRQRRR, SEQ ID NO: 11 754).

In a particular embodiment, the invention relates to a pharmaceutical composition as defined above, wherein said miPEP, or said fragment of said miPEP, is fused in N-ter or in C-ter to the TAT peptide (YGRKKRRQRRR, SEQ ID NO: 11 754).

In a particular embodiment, the invention relates to a pharmaceutical composition as defined above, wherein said miPEP fragment is selected from:

miPEP//145-2-TAT consisting of SEQ ID NO: 11 755
(MVGLNPPLWQYGRKKRRQRRR),
miPEP//125a-1-TAT consisting of SEQ ID NO: 11 756
(MSLCLSPSLTYGRKKRRQRRR),
and
miPEP//15a-16-1-TAT consisting of SEQ ID NO: 11 757
(MFKHRFFYMHYGRKKRRQRRR).

In a particular embodiment, the invention relates to a pharmaceutical composition as defined above, wherein said miPEP, or said fragment of said miPEP, is fused to penetratin, to a polyhistidine peptide (in particular a peptide of at least 6 histidine residues) or to a polyarginine peptide (in particular a peptide of 9 arginine residues).

In a particular embodiment, the invention relates to a pharmaceutical composition as defined above, wherein said miPEP, or said fragment of said miPEP, is linked to one or more palmitic acid molecules.

The amount of miPEP present in the composition or used for the treatment of a pathology may vary depending on whether or not the miPEP is modified with a molecule facilitating cell penetration.

In another aspect, the invention relates to a miPEP from 3 to 500 amino acids encoded by a nucleotide sequence contained in the primary transcript of a miR, or a fragment of said miPEP, said miPEP being capable of modulating the accumulation of said miR in a eukaryotic cell, for its use as a drug.

In a particular embodiment, the invention relates to a miPEP from 3 to 500 amino acids encoded by a nucleotide sequence contained in the primary transcript of a miR, or a fragment of said miPEP, said miPEP being capable of modulating the accumulation of said miR in a eukaryotic cell, which miR regulates expression of at least one gene involved in a pathology, for its use as a drug.

In the invention, a gene is involved in a pathology if the expression of said gene varies significantly between a patient suffering from said pathology and a healthy individual.

In a particular embodiment, the invention relates to a miPEP, or a fragment of said miPEP, for its use as defined above, said miPEP being identified, or as identified, by implementing of a process for identifying a miPEP modulating the accumulation of a miR involved in a pathology, comprising:
a) a step of detecting an open reading frame from 12 to 1 503 nucleotides contained in the sequence of the primary transcript of said miR, then
b) a step of comparison between:
   i. the accumulation of said miR in a specified eukaryotic cell expressing the primary transcript of said miR, in the presence of a peptide encoded by a nucleotide sequence that is identical or degenerate relative to that of said open reading frame, said peptide being present in the cell independently of transcription of the primary transcript of said miR, and
   ii. the accumulation of said miR in a eukaryotic cell of the same type as the aforesaid specified eukaryotic cell expressing the primary transcript of said miR, in the absence of said peptide,
      in which a modulation of the accumulation of said miR in the presence of said peptide relative to the accumulation of said miR in the absence of said peptide indicates the existence of a miPEP, the latter being encoded by said open reading frame and being capable of modulating the accumulation of said miR involved in a pathology.

In the invention, a miR is involved in a pathology if said miR is capable of regulating the expression of a gene involved in said pathology and/or of reducing the symptoms of said pathology.

In a particular embodiment, the invention relates to a miPEP, or a fragment of said miPEP, for its use as defined above, said miPEP being encoded by a nucleotide sequence contained in the primary transcript of a miR selected from those presented in Tables 2, 3, 4, 5 and 6.

In a particular embodiment, the invention relates to a miPEP, or a fragment of said miPEP, for its use as defined above, said miPEP being encoded by a nucleotide sequence contained in the primary transcript of a miR selected from the group consisting of:

hsa-mir-103-1, hsa-mir-103-2, hsa-mir-107, hsa-mir-122, hsa-mir-155, hsa-mir-21, hsa-mir-33, hsa-mir-15a, hsa-miR-15b, hsa-mir-451, hsa-mir-92a-1, hsa-mir-92a-2, hsa-mir-92b, hsa-mir-208, hsa-mir-208b, hsa-mir-29a, hsa-mir-29b-1, hsa-mir-29b-2, hsa-mir-29c, hsa-mir-143, hsa-mir-145, hsa-mir-206, hsa-mir-378, hsa-mir-34a, hsa-mir-34b, hsa-mir-34c, hsa-let-7, hsa-mir-16-1, hsa-mir-16-2 and hsa-mir-125a.

In a particular embodiment, the invention relates to a miPEP, or a fragment of said miPEP, for its use as defined above, said miPEP being encoded by a nucleotide sequence contained in the primary transcript of a miR selected from the group consisting of: hsa-mir-125a, hsa-mir-15a-16-1 and hsa-mir145.

In a particular embodiment, the invention relates to a miPEP, or a fragment of said miPEP, for its use as defined above, said miPEP being selected from the group consisting of SEQ ID NO:

2q-1, q varying from 1 to 5 875.

In a particular embodiment, the invention relates to a miPEP, or a fragment of said miPEP, for its use as defined above, said miPEP being selected from the group consisting of: miPEP 145-2 (SEQ ID NO: 11745), miPEP 125a-1 (SEQ ID NO: 11747) and miPEP 15a-16-1 (SEQ ID NO: 11 749).

In a particular embodiment, the invention relates to a miPEP, or a fragment of said miPEP, for its use as defined above, said miPEP fragment being a fragment of miPEP 145-2, preferably consisting of SEQ ID NO: 11 751.

In a particular embodiment, the invention relates to a miPEP, or a fragment of said miPEP, for its use as defined above, said miPEP fragment being miPEP//145-2 consisting of SEQ ID NO: 11 751.

In a particular embodiment, the invention relates to a miPEP, or a fragment of said miPEP, for its use as defined above, said miPEP fragment being a fragment of miPEP 125a-1, preferably consisting of SEQ ID NO: 11 752.

In a particular embodiment, the invention relates to a miPEP, or a fragment of said miPEP, for its use as defined above, said miPEP fragment being miPEP//125a-1 consisting of SEQ ID NO: 11 752.

In a particular embodiment, the invention relates to a miPEP, or a fragment of said miPEP, for its use as defined above, said miPEP fragment being a fragment of miPEP 15a-16-1, preferably consisting of SEQ ID NO: 11 753.

In a particular embodiment, the invention relates to a miPEP, or a fragment of said miPEP, for its use as defined above, said miPEP fragment being miPEP//15a-16-1 consisting of SEQ ID NO: 11 753.

In a particular embodiment, the invention relates to a miPEP, or a fragment of said miPEP, for its use as defined above, wherein said miPEP, or said fragment of said miPEP, is fused to the TAT peptide.

In a particular embodiment, the invention relates to a miPEP fragment for its use as defined above, said miPEP fragment being selected from:

miPEP//145-2-TAT consisting of SEQ ID NO: 11 755
(MVGLNPPLWQYGRKKRRQRRR),
miPEP//125a-1-TAT consisting of SEQ ID NO: 11 756
(MSLCLSPSLTYGRKKRRQRRR),
and
miPEP//15a-16-1-TAT consisting of SEQ ID NO: 11 757
(MFKHRFFYMHYGRKKRRQRRR).

In a particular embodiment, the invention relates to a miPEP, or a fragment of said miPEP, for its use as defined above, wherein said miPEP, or said fragment of said miPEP, being fused to penetratin, to a polyhistidine peptide (in particular a peptide of at least 6 histidine residues) or to a polyarginine peptide (in particular a peptide of 9 arginine residues).

In a particular embodiment, the invention relates to a miPEP, or a fragment of said miPEP, for its use as defined above, wherein said miPEP, or said fragment of said miPEP, being linked to one or more palmitic acid molecules.

Another aspect of the invention relates to a miPEP from 3 to 500 amino acids encoded by a nucleotide sequence contained in the primary transcript of a miR, or a fragment of said miPEP, said miPEP being capable of modulating the accumulation of said miR in a eukaryotic cell, for its use in the treatment of a pathology selected from: cancer, bacterial infections, mycoses, cardiovascular diseases, hereditary congenital diseases, skin diseases, eye diseases, diseases of the digestive system, diseases of the endocrine system, diseases of the nervous system, diseases related to viruses, diseases related to nutrition and metabolism, lymphatic diseases and hemopathies, neonatal and hereditary diseases, respiratory diseases, urogenital diseases in humans, urogenital diseases in women, disorders of the immune system, musculoskeletal disorders, diseases or trauma of bone tissue or cartilage tissue.

Another aspect of the invention relates to a miPEP from 3 to 500 amino acids encoded by a nucleotide sequence contained in the primary transcript of a miR, or a fragment of said miPEP, said miPEP being capable of modulating the accumulation of said miR in a eukaryotic cell, for its use in the treatment of a disease or trauma of bone tissue or cartilage tissue.

In the invention, the term “bone tissue disease” refers to any disease reducing bone capital, such as osteoporosis.

In the invention, the term “bone tissue trauma” refers to a break in continuity or fracture of a bone.

In a particular embodiment, the invention relates to a miPEP from 3 to 500 amino acids encoded by a nucleotide sequence contained in the primary transcript of a miR, or a fragment of said miPEP, said miPEP being capable of modulating the accumulation of said miR in a eukaryotic cell, for its use as defined above, which miR regulates expression of at least one gene involved in a pathology.

In a particular embodiment, the invention relates to a miPEP, or a fragment of said miPEP, for its use as defined above, said miPEP being identified, or as identified, by implementing of a process for identifying a miPEP modulating the accumulation of a miR involved in a pathology, comprising:
a) a step of detecting an open reading frame from 12 to 1 503 nucleotides contained in the sequence of the primary transcript of said miR, then
b) a step of comparison between:
   i. the accumulation of said miR in a specified eukaryotic cell expressing the primary transcript of said miR, in the presence of a peptide encoded by a nucleotide sequence that is identical or degenerate relative to that of said open reading frame, said peptide being present in the cell independently of transcription of the primary transcript of said miR, and
   ii. the accumulation of said miR in a eukaryotic cell of the same type as the aforesaid specified eukaryotic cell expressing the primary transcript of said miR, in the absence of said peptide,
      in which a modulation of the accumulation of said miR in the presence of said peptide relative to the accumulation of said miR in the absence of said peptide indicates the existence of a miPEP, the latter being encoded by said open reading frame and being capable of modulating the accumulation of said miR involved in a pathology.

In a particular embodiment, the invention relates to a miPEP, or a fragment of said miPEP, for its use as defined above, said miPEP being selected from the group consisting of SEQ ID NO: 2q-1, q varying from 1 to 5 875.

In a particular embodiment, the invention relates to a miPEP, or a fragment of said miPEP, for its use as defined above, said miPEP being selected from the group consisting of: miPEP 145-2 (SEQ ID NO: 11745), miPEP 125a-1 (SEQ ID NO: 11747) and miPEP 15a-16-1 (SEQ ID NO: 11 749).

In a particular embodiment, the invention relates to a miPEP, or a fragment of said miPEP, for its use as defined above, said miPEP fragment being a fragment of miPEP 145-2, preferably consisting of SEQ ID NO: 11 751.

In a particular embodiment, the invention relates to a miPEP, or a fragment of said miPEP, for its use as defined above, said miPEP fragment being miPEP//145-2 consisting of SEQ ID NO: 11 751.

In a particular embodiment, the invention relates to a miPEP, or a fragment of said miPEP, for its use as defined above, said miPEP fragment being a fragment of miPEP 125a-1, preferably consisting of SEQ ID NO: 11 752.

In a particular embodiment, the invention relates to a miPEP, or a fragment of said miPEP, for its use as defined above, said miPEP fragment being miPEP//125a-1 consisting of SEQ ID NO: 11 752.

In a particular embodiment, the invention relates to a miPEP, or a fragment of said miPEP, for its use as defined above, said miPEP fragment being a fragment of miPEP 15a-16-1, preferably consisting of SEQ ID NO: 11 753.

In a particular embodiment, the invention relates to a miPEP, or a fragment of said miPEP, for its use as defined above, said miPEP fragment being miPEP//15a-16-1 consisting of SEQ ID NO: 11 753.

In a particular embodiment, the invention relates to a miPEP, or a fragment of said miPEP, for its use as defined above, wherein said miPEP, or said fragment of said miPEP, is fused to the TAT peptide.

In a particular embodiment, the invention relates to a miPEP fragment for its use as defined above, said miPEP fragment being selected from:

miPEP//145-2-TAT consisting of SEQ ID NO: 11 755
(MVGLNPPLWQYGRKKRRQRRR),
miPEP//125a-1-TAT consisting of SEQ ID NO: 11 756
(MSLCLSPSLTYGRKKRRQRRR),
and
miPEP//15a-16-1-TAT consisting of SEQ ID NO: 11 757
(MFKHRFFYMHYGRKKRRQRRR).

In a particular embodiment, the invention relates to a miPEP, or a fragment of said miPEP, for its use as defined above, wherein said miPEP, or said fragment of said miPEP, being fused to penetratin, to a polyhistidine peptide (in particular a peptide of at least 6 histidine residues) or to a polyarginine peptide (in particular a peptide of 9 arginine residues).

In a particular embodiment, the invention relates to a miPEP, or a fragment of said miPEP, for its use as defined above, wherein said miPEP, or said fragment of said miPEP, being linked to one or more palmitic acid molecules.

Tables 2 to 6 present the different miRs and the pathologies in which they are involved.

In a particular embodiment, the invention relates to a miPEP, or a fragment of said miPEP, for its use as defined above, said pathology being a cancer, said miPEP being encoded by a nucleotide sequence contained in the primary transcript of a miR selected from the cancer-related miRs in Table 2, particularly among the cancer-related miRs in Table 5.

In a particular embodiment, the invention relates to a miPEP, or a fragment of said miPEP, for its use as defined above, said pathology being a bacterial infection or mycosis, said miPEP being encoded by a nucleotide sequence contained in the primary transcript of a miR selected from the bacterial infection- and mycosis-related miRs in Table 2, particularly among the bacterial infection- and mycosis-related miRs in Table 5.

In a particular embodiment, the invention relates to a miPEP, or a fragment of said miPEP, for its use as defined above, said pathology being a cardiovascular disease, said miPEP being encoded by a nucleotide sequence contained in the primary transcript of a miR selected from the cardiovascular disease-related miRs in Table 2, particularly among the cardiovascular disease-related miRs in Table 5.

In a particular embodiment, the invention relates to a miPEP, or a fragment of said miPEP, for its use as defined above, said pathology being a hereditary congenital disease, said miPEP being encoded by a nucleotide sequence contained in the primary transcript of a miR selected from the hereditary congenital disease-related miRs in Table 2, particularly among the hereditary congenital disease-related miRs in Table 5.

In a particular embodiment, the invention relates to a miPEP, or a fragment of said miPEP, for its use as defined above, said pathology being a skin disease, said miPEP being encoded by a nucleotide sequence contained in the primary transcript of a miR selected from the skin disease-related miRs in Table 2, particularly among the skin disease-related miRs in Table 5.

In a particular embodiment, the invention relates to a miPEP, or a fragment of said miPEP, for its use as defined above, said pathology being a eye disease, said miPEP being encoded by a nucleotide sequence contained in the primary transcript of a miR selected from the eye disease-related miRs in Table 2, particularly among the eye disease-related miRs in Table 5.

In a particular embodiment, the invention relates to a miPEP, or a fragment of said miPEP, for its use as defined above, said pathology being a disease of the digestive system, said miPEP being encoded by a nucleotide sequence contained in the primary transcript of a miR selected from disease of the digestive system-related miRs in Table 3, particularly among disease of the digestive system-related miRs in Table 5.

In a particular embodiment, the invention relates to a miPEP, or a fragment of said miPEP, for its use as defined above, said pathology being a disease of the endocrine system, said miPEP being encoded by a nucleotide sequence contained in the primary transcript of a miR selected from disease of the endocrine system-related miRs in Table 3, particularly among disease of the endocrine system-related miRs in Table 6.

In a particular embodiment, the invention relates to a miPEP, or a fragment of said miPEP, for its use as defined above, said pathology being a disease of the nervous system, said miPEP being encoded by a nucleotide sequence contained in the primary transcript of a miR selected from disease of the nervous system-related miRs in Table 3, particularly among disease of the nervous system-related miRs in Table 6.

In a particular embodiment, the invention relates to a miPEP, or a fragment of said miPEP, for its use as defined above, said pathology being a disease linked to a virus, said miPEP being encoded by a nucleotide sequence contained in the primary transcript of a miR selected from disease linked to a virus-related miRs in Table 3, particularly among disease linked to a virus-related miRs in Table 6.

In a particular embodiment, the invention relates to a miPEP, or a fragment of said miPEP, for its use as defined above, said pathology being a disease related to nutrition and metabolism, said miPEP being encoded by a nucleotide sequence contained in the primary transcript of a miR selected from disease related to nutrition and metabolism-related miRs in Table 3, particularly among disease related to nutrition and metabolism-related miRs in Table 6.

In a particular embodiment, the invention relates to a miPEP, or a fragment of said miPEP, for its use as defined above, said pathology being a lymphatic disease or hemopathy, said miPEP being encoded by a nucleotide sequence contained in the primary transcript of a miR selected from lymphatic disease or hemopathy-related miRs in Table 3, particularly among lymphatic disease or hemopathy-related miRs in Table 6.

In a particular embodiment, the invention relates to a miPEP, or a fragment of said miPEP, for its use as defined above, said pathology being a neonatal and hereditary disease, said miPEP being encoded by a nucleotide sequence contained in the primary transcript of a miR selected from neonatal and hereditary disease -related miRs in Table 3, particularly among neonatal and hereditary disease-related miRs in Table 6.

In a particular embodiment, the invention relates to a miPEP, or a fragment of said miPEP, for its use as defined above, said pathology being a respiratory disease, said miPEP being encoded by a nucleotide sequence contained in the primary transcript of a miR selected from respiratory disease-related miRs in Table 4, particularly among respiratory disease-related miRs in Table 6.

In a particular embodiment, the invention relates to a miPEP, or a fragment of said miPEP, for its use as defined above, said pathology being a urogenital disease in men, said miPEP being encoded by a nucleotide sequence contained in the primary transcript of a miR selected from urogenital disease in men-related miRs in Table 4, particularly among urogenital disease in men-related miRs in Table 6.

In a particular embodiment, the invention relates to a miPEP, or a fragment of said miPEP, for its use as defined above, said pathology being a urogenital disease in women, said miPEP being encoded by a nucleotide sequence contained in the primary transcript of a miR selected from urogenital disease in women-related miRs in Table 4, particularly among urogenital disease in women-related miRs in Table 6.

In a particular embodiment, the invention relates to a miPEP, or a fragment of said miPEP, for its use as defined above, said pathology being a disorder of the immune system, said miPEP being encoded by a nucleotide sequence contained in the primary transcript of a miR selected from disorder of the immune system-related miRs in Table 4, particularly among disorder of the immune system-related miRs in Table 6.

In a particular embodiment, the invention relates to a miPEP, or a fragment of said miPEP, for its use as defined above, said pathology being a musculoskeletal disorder, said miPEP being encoded by a nucleotide sequence contained in the primary transcript of a miR selected from musculoskeletal disorder-related miRs in Table 4, particularly among musculoskeletal disorder-related miRs in Table 6.

In a particular embodiment, the invention relates to a miPEP, or a fragment of said miPEP, for its use as defined above, said pathology being a metabolism disorder, said miR being selected from: hsa-miR-103-1, hsa-miR-103-2 and hsa-mir-107, said miPEP being preferably selected from SEQ ID NOs: 73, 75, 77, 79, 81, 83, 85, 87, 2 547, 2 549, 2 551, 2 553, 3 921, 3 923, 3 925 and 3 927.

In a particular embodiment, the invention relates to a miPEP, or a fragment of said miPEP, for its use as defined above, said pathology being a hepatitis C virus infection, said miR being the hsa-miR-122, said miPEP being preferably selected from SEQ ID NOs: 97, 99, 101, 103, 2 561, 2 563, 2 565 and 2 567.

In a particular embodiment, the invention relates to a miPEP, or a fragment of said miPEP, for its use as defined above, said pathology being an inflammatory disease, said miR being the hsa-miR-155, said miPEP being preferably selected from SEQ ID NOs: 4 433, 4 435, 4 437, 4 439, 4 441, 4 443, 4 445, 4 447, 8 377, 8 379, 8 381 and 8 383.

In a particular embodiment, the invention relates to a miPEP, or a fragment of said miPEP, for its use as defined above, said pathology being a fibrosis, said miR being the hsa-miR-21, said miPEP being preferably selected from SEQ ID NOs: 497, 499, 501, 503, 2 793, 2 795, 2 797, 2 799, 3 425, 3 427, 3 429, 3 431, 4 937, 4 939, 4 941, 4 943, 8 545, 8 547, 8 549, 8 551, 10 801, 10 803, 10 805 and 10 807.

In a particular embodiment, the invention relates to a miPEP, or a fragment of said miPEP, for its use as defined above, said pathology being atherosclerosis, said miR being the hsa-miR-33, said miPEP being preferably selected from SEQ ID NOs: 5 609, 5 611, 5 613, 5 615, 8 673, 8 675, 8 677 and 8 679.

In a particular embodiment, the invention relates to a miPEP, or a fragment of said miPEP, for its use as defined above, said pathology being a cardiovascular disease, said miR being selected from: hsa-miR-15a and has-miR-15b, said miPEP being preferably selected from SEQ ID NOs: 4 449, 4 451, 4 453, 4 455, 4 457, 4 459, 4 461, 4 463, 4 465, 4 467, 4 469 and 4 471.

In a particular embodiment, the invention relates to a miPEP, or a fragment of said miPEP, for its use as defined above, said pathology being a myeloproliferative disease, said miR being the hsa-miR-451, said miPEP being preferably selected from SEQ ID NOs: 1 017, 1 019, 1 021, 1 023, 3 609, 3 611, 3 613, 3 615, 6 113, 6 115, 6 117 and 6 119.

In a particular embodiment, the invention relates to a miPEP, or a fragment of said miPEP, for its use as defined above, said pathology being linked to neoangiogenesis, said miR being selected from: hsa-mir-92a-1, hsa-mir-92a-2 and hsa-mir-92b, said miPEP being preferably selected from SEQ ID NOs: 1 777, 1 779, 1 781, 1 783, 1 785, 1 787, 1 789, 1 791, 8 033, 8 035, 8 037, 8 039, 8 041, 8 043, 8 045, 8 047, 8 049, 8 051, 8 053, 8 055, 9 225, 9 227, 9 229, 9 231, 9 233, 9 235, 9 237, 9 239, 9 241, 9 243, 9 245, 9 247, 10 345, 10 347, 10 349, 10 351, 10 353, 10 355, 10 357, 10 359, 10 361, 10 363, 10 365, 10 367, 11 489, 11 491, 11 493, 11 495, 11 497, 11 499, 11 501, 11 503, 11 505, 11 507, 11 509 and 11 511.

In a particular embodiment, the invention relates to a miPEP, or a fragment of said miPEP, for its use as defined above, said pathology being a cardiac disorder, said miR being selected from: hsa-mir-208 and hsa-mir-208b, said miPEP being preferably selected from SEQ ID NOs: 4 889, 4 891, 4 893, 4 895, 4 897, 4 899, 4 901, 4 903, 4 905, 4 907, 4 909, 4 911, 4 913, 4 915, 4 917 and 4919.

In a particular embodiment, the invention relates to a miPEP, or a fragment of said miPEP, for its use as defined above, said pathology being a pulmonary fibrosis, said miR being the hsa-mir-208, said miPEP being preferably selected from SEQ ID NOs: 4 889, 4 891, 4 893, 4 895, 4 897, 4 899, 4 901, 4 903, 4 905, 4 905, 4 907, 4 909, 4 911, 4 913, 4 915, 4 917 and 4 919.

In a particular embodiment, the invention relates to a miPEP, or a fragment of said miPEP, for its use as defined above, said pathology being a cancer, said miR being the hsa-let-7, said miPEP being preferably selected from SEQ ID NO: 2q-1, q varying from 1 to 28, from 1 225 to 1 260, from 1 561 to 1 576, from 1 865 to 1 916, from 4 065 to 4 100, from 4 645 to 4 668, from 5 197 to 5 228.

In a particular embodiment, the invention relates to a miPEP, or a fragment of said miPEP, for its use as defined above, said pathology being a cardiovascular disease, in particular the ischemia-reperfusion syndrome, said miR being the hsa-miR-7, said miPEP being preferably selected from SEQ ID NOs: 3 089, 3 091, 3 093, 3 095, 3 689, 3 691, 3 693, 3 695, 7 881, 7 883, 7 885, 7 887, 7 889, 7 891, 7 893, 7 895, 7 897, 7 899, 7 901, 7 903, 9 209, 9 211, 9 213, 9 215, 11 473, 11 475, 11 477 and 11 479.

In a particular embodiment, the invention relates to a miPEP, or a fragment of said miPEP, for its use as defined above, said pathology being a hepatitis C virus infection, said miR being the hsa-mir-181c, said miPEP being preferably selected from SEQ ID NOs: 321, 323, 325, 327, 2 657, 2 659, 2 661, 2 663, 4 537, 4 539, 4 541, 4 543, 8 409, 8 411, 8 413, 8 415, 9 497, 9 499, 9 501 and 9 503.

In a particular embodiment, the invention relates to a miPEP, or a fragment of said miPEP, for its use as defined above, said pathology being a pulmonary fibrosis, said miR being the hsa-miR-29, said miPEP being preferably selected from SEQ ID NOs: 633, 635, 637, 639, 641, 643, 645, 647, 649, 651, 653, 655, 657, 659, 661, 663, 2 905, 2 907, 2 909, 2 911, 2 913, 2 915, 2 917, 2 919, 3 529, 3 531, 3 533, 3 535, 3 537, 3 539, 3 541, 3 543, 5 281, 5 283, 5 285, 5 287, 5 289, 5 291, 5 293, 5 295, 5 297, 5 299, 5 301, 5 303, 5 305, 5 307, 5 309, 5 311, 11 657, 11 659, 11 661, 11 663, 11 665, 11 667, 11 669 and 11 671.

In a particular embodiment, the invention relates to a miPEP for its use in the treatment of a disease or trauma of bone tissue or cartilage tissue, said miPEP being selected from the group of miPEPs consisting of: miPEP 145-2 (SEQ ID NO: 11 745), miPEP 125a-1 (SEQ ID NO: 11 747) and miPEP 15a-16-1 (SEQ ID NO: 11 749).

In a particular embodiment, the invention relates to a miPEP fragment for its use in the treatment of a disease or trauma of bone tissue or cartilage tissue, said miPEP fragment being a fragment of the miPEP 145-2, preferably consisting of SEQ ID NO: 11 751.

In a particular embodiment, the invention relates to a miPEP fragment for its use in the treatment of a disease or trauma of bone tissue or cartilage tissue, said miPEP fragment being the miPEP//145-2 consisting of SEQ ID NO: 11 751.

In a particular embodiment, the invention relates to a miPEP fragment for its use in the treatment of a disease or trauma of bone tissue or cartilage tissue, said miPEP fragment being a fragment of the miPEP 125a-1, preferably consisting of SEQ ID NO: 11 752.

In a particular embodiment, the invention relates to a miPEP fragment for its use in the treatment of a disease or trauma of bone tissue or cartilage tissue, said miPEP fragment being the miPEP//125a-1 consisting of SEQ ID NO: 11 752.

In a particular embodiment, the invention relates to a miPEP fragment for its use in the treatment of a disease or trauma of bone tissue or cartilage tissue, said miPEP fragment being a fragment of the miPEP 15a-16-1, preferably consisting of SEQ ID NO: 11 753.

In a particular embodiment, the invention relates to a miPEP fragment for its use in the treatment of a disease or trauma of bone tissue or cartilage tissue, said miPEP fragment being the miPEP//15a-16-1 consisting of SEQ ID NO: 11 754.

In a particular embodiment, the invention relates to a miPEP fragment for its use in the treatment of a disease or trauma of bone tissue or cartilage tissue, said miPEP fragment being selected from:

miPEP//145-2-TAT consisting of SEQ ID NO: 11 755
(MVGLNPPLWQYGRKKRRQRRR),
miPEP//125a-1-TAT consisting of SEQ IDNO: 11 756
(MSLCLSPSLTYGRKKRRQRRR),
and
miPEP//15a-16-1-TAT consisting of SEQ ID NO: 11 757
(MFKHRFFYMHYGRKKRRQRRR).

Another aspect of the invention relates to process for identifying a miPEP, or a fragment of said miPEP, modulating the accumulation of a miR involved in a pathology, said miPEP being encoded by a nucleotide sequence contained in the primary transcript of a miR, comprising:
a) a step of detecting an open reading frame from 12 to 1 503 nucleotides contained in the sequence of the primary transcript of said miR, then
b) a step of comparison between:
   i. the accumulation of said miR in a specified eukaryotic cell expressing the primary transcript of said miR, in the presence of a peptide encoded by a nucleotide sequence that is identical or degenerate relative to that of said open reading frame, said peptide being present in the cell independently of transcription of the primary transcript of said miR, and
   ii. the accumulation of said miR in a eukaryotic cell of the same type as the aforesaid specified eukaryotic cell expressing the primary transcript of said miR, in the absence of said peptide,
      in which a modulation of the accumulation of said miR in the presence of said peptide relative to the accumulation of said miR in the absence of said peptide indicates the existence of a miPEP, the latter being encoded by said open reading frame and being capable of modulating the accumulation of said miR involved in a pathology.

Another aspect of the invention also relates to a miPEP from 101 to 500 amino acids, or a fragment of said miPEP, encoded by a nucleotide sequence contained in the primary transcript of a microRNA, said miPEP being capable of modulating the accumulation of said miR in a eukaryotic cell.

In particular, a miPEP may have a size from 200 to 500 amino acids, from 300 to 500 amino acids or from 400 to 500 amino acids.

In particular, a miPEP may have a size of 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 amino acids.

In a particular embodiment, the invention relates to a miPEP selected from the group consisting of SEQ ID NO: 2q-1, q varying from 1 to 5 875, or a fragment of said miPEP.

In a particular embodiment, the invention relates to a miPEP selected from the group consisting of: miPEP 145-2 (SEQ ID NO: 11 745), miPEP 125a-1 (SEQ ID NO: 11 747) and miPEP 15a-16-1 (SEQ ID NO: 11 749).

In a particular embodiment, the invention relates to a fragment of miPEP 145-2, preferably consisting of SEQ ID NO: 11 751.

In a particular embodiment, the invention relates to miPEP//145-2 consisting of SEQ ID NO: 11 751.

In a particular embodiment, the invention relates to a fragment of miPEP 125a-1, preferably consisting of SEQ ID NO: 11 752.

In a particular embodiment, the invention relates to miPEP//125a-1 consisting of SEQ ID NO: 11 752.

In a particular embodiment, the invention relates to a fragment of miPEP 15a-16-1, preferably consisting of SEQ ID NO: 11 753.

In a particular embodiment, the invention relates to miPEP//15a-16-1 consisting of SEQ ID NO: 11 753.

In a particular embodiment, the invention relates to a miPEP, or a fragment of said miPEP, vectorized or encapsulated.

In a particular embodiment, the invention relates to a miPEP, or a fragment of said miPEP, fused to a peptide facilitating the entry into the cell of the miPEP, or miPEP fragment.

In a particular embodiment, the invention relates to a miPEP, or a fragment of said miPEP, wherein said miPEP, or said fragment of said miPEP, fused to the TAT peptide (YGRKKRRQRRR, SEQ ID NO: 11 754).

In a particular embodiment, the invention relates to a miPEP fragment, said miPEP fragment being selected from:

miPEP//145-2-TAT consisting of SEQ ID NO: 11 755
(MVGLNPPLWQYGRKKRRQRRR),
miPEP//125a-1-TAT consisting of SEQ ID NO: 11 756
(MSLCLSPSLTYGRKKRRQRRR),
and
miPEP//15a-16-1-TAT consisting of SEQ ID NO: 11 757
(MFKHRFFYMHYGRKKRRQRRR).

In a particular embodiment, the invention relates to a miPEP, or a fragment of said miPEP, as defined above, fused to penetratin, to a polyhistidine peptide (in particular a peptide of at least 6 histidine residues) or to a polyarginine peptide (in particular a peptide of 9 arginine residues).

In a particular embodiment, the invention relates to a miPEP, or a fragment of said miPEP, as defined above, linked to one or more palmitic acid molecules. In another aspect, the invention relates to a composition comprising a miPEP or a fragment of said miPEP.

In a particular embodiment, the invention relates to a composition as defined above, wherein said miPEP is selected from the group of miPEP consisting of SEQ ID NO: 2q-1, q varying from 1 to 5 875.

In a particular embodiment, the invention relates to a composition as defined above, wherein said miPEP is selected from the group of miPEP consisting of: miPEP 145-2 (SEQ ID NO: 11745), miPEP 125a-1 (SEQ ID NO: 11747) and miPEP 15a-16-1 (SEQ ID NO: 11 749).

In a particular embodiment, the invention relates to a composition as defined above, wherein said miPEP fragment is a fragment of miPEP 145-2, preferably consisting of SEQ ID NO: 11 751.

In a particular embodiment, the invention relates to a composition as defined above, wherein said miPEP fragment is the miPEP//145-2 consisting of SEQ ID NO: 11 751.

In a particular embodiment, the invention relates to a composition as defined above, wherein said miPEP fragment is a fragment of miPEP 125a-1, preferably consisting of SEQ ID NO: 11 752.

In a particular embodiment, the invention relates to a composition as defined above, wherein said miPEP fragment is the miPEP//125a-1 consisting of SEQ ID NO: 11 752.

In a particular embodiment, the invention relates to a composition as defined above, wherein said miPEP fragment is a fragment of miPEP 15a-16-1, preferably consisting of SEQ ID NO 11 753.

In a particular embodiment, the invention relates to a composition as defined above, wherein said miPEP fragment is the miPEP//15a-16-1 consisting of SEQ ID NO: 11 753.

In a particular embodiment, the invention relates to a composition as defined above, wherein said miPEP fragment being selected from:

miPEP//145-2-TAT consisting of SEQ ID NO: 11 755
(MVGLNPPLWQYGRKKRRQRRR),
miPEP//125a-1-TAT consisting of SEQ ID NO: 11 756
(MSLCLSPSLTYGRKKRRQRRR),
and
miPEP//15a-16-1-TAT consisting of SEQ ID NO: 11 757
(MFKHRFFYMHYGRKKRRQRRR).

In a particular embodiment, the invention relates to a composition as defined above, wherein said composition additionally contains growth factors and/or cell differentiation factors.

In a particular embodiment, the invention relates to a composition as defined above, wherein said composition additionally contains BMP4.

In a particular embodiment, the invention relates to a composition as defined above, wherein said composition additionally contains cell culture medium.

In another aspect, the invention relates to the in vitro or ex vivo use of a miPEP from 3 to 500 amino acids encoded by a nucleotide sequence contained in the primary transcript of a miR, or a fragment of said miPEP, said miPEP being capable of modulating the accumulation of said miR in a eukaryotic cell, to promote cell differentiation of cells.

In a particular embodiment, the invention relates to the in vitro or ex vivo use of a miPEP, or a fragment of said miPEP, to promote cell differentiation of mesenchymal stem cells (also called bone marrow stromal cells or mesenchymal stromal cells).

In a particular embodiment, the invention relates to the in vitro or ex vivo use of a miPEP, or a fragment of said miPEP, to promote cell differentiation of mesenchymal stem cells in osteoblasts.

In a particular embodiment, the invention relates to the use as defined above, wherein cells are cultured in the presence of said miPEP, or a fragment of said miPEP.

In a particular embodiment, the invention relates to the use as defined above, wherein cells are cultured in the presence of said miPEP, or a fragment of said miPEP, and in the presence of growth factors and/or cell differentiation factors.

In a particular embodiment, the invention relates to the use as defined above, wherein cells are cultured in the presence of said miPEP, or a fragment of said miPEP, and in the presence of BMP4 (Bone Morphogenetic Protein 4).

In a particular embodiment, the invention relates to the use as defined above, wherein said miPEP is selected from the group of miPEPs consisting of SEQ ID NO: 2q-1, q varying from 1 to 5 875.

In a particular embodiment, the invention relates to the use as defined above, wherein said miPEP selected from the group consisting of: miPEP 145-2 (SEQ ID NO: 11 745), miPEP 125a-1 (SEQ ID NO: 11 747) and miPEP 15a-16-1 (SEQ ID NO: 11 749).

In a particular embodiment, the invention relates to the use as defined above, wherein said fragment is a fragment of miPEP 145-2, preferably consisting of SEQ ID NO: 11 751.

In a particular embodiment, the invention relates to the use as defined above, wherein said fragment is the miPEP//145-2 consisting of SEQ ID NO: 11 751.

In a particular embodiment, the invention relates to the use as defined above, wherein said fragment is a fragment of miPEP 125a-1, preferably consisting of SEQ ID NO: 11 752.

In a particular embodiment, the invention relates to the use as defined above, wherein said fragment is the miPEP//125a-1 consisting of SEQ ID NO: 11 752.

In a particular embodiment, the invention relates to the use as defined above, wherein said fragment is a fragment of miPEP 15a-16-1, preferably consisting of SEQ ID NO: 11 753.

In a particular embodiment, the invention relates to the use as defined above, wherein said fragment is the miPEP//15a-16-1 consisting of SEQ ID NO: 11 753.

In a particular embodiment, the invention relates to the use as defined above, wherein said miPEP fragment is selected from:

miPEP//145-2-TAT consisting of SEQ ID NO: 11 755
(MVGLNPPLWQYGRKKRRQRRR),
miPEP//125a-1-TAT consisting of SEQ ID NO: 11 756
(MSLCLSPSLTYGRKKRRQRRR),
and
miPEP//15a-16-1-TAT consisting of SEQ ID NO: 11 757
(MFKHRFFYMHYGRKKRRQRRR).

In a particular embodiment, the invention relates to the in vitro or ex vivo use of a miPEP, or a fragment of said miPEP, to induce or promote bone formation.

In a particular embodiment, the invention relates to the in vitro or ex vivo use of a miPEP, or a fragment of said miPEP, to form a mineralized bone matrix.

In a particular embodiment, the invention relates to the in vitro or ex vivo use of a miPEP, or a fragment of said miPEP, to promote bone formation with the help of a bone biomaterial.

In a particular embodiment, the invention relates to the use as defined above, wherein mesenchymal stem cells are cultured in the presence of said miPEP, or a fragment of said miPEP.

In a particular embodiment, the invention relates to the use as defined above, wherein mesenchymal stem cells are cultured in the presence of said miPEP, or a fragment of said miPEP, and in the presence of growth factors and/or cell differentiation factors.

In a particular embodiment, the invention relates to the use as defined above, wherein mesenchymal stem cells are cultured in the presence of said miPEP, or a fragment of said miPEP, and in the presence of BMP4.

In a particular embodiment, the invention relates to the use as defined above, wherein said miPEP is selected from the group of miPEPs consisting of SEQ ID NO: 2q-1, q varying from 1 to 5 875.

In a particular embodiment, the invention relates to the use as defined above, wherein said miPEP selected from the group consisting of: miPEP 145-2 (SEQ ID NO: 11 745), miPEP 125a-1 (SEQ ID NO: 11 747) and miPEP 15a-16-1 (SEQ ID NO: 11 749).

In a particular embodiment, the invention relates to the use as defined above, wherein said fragment is a fragment of miPEP 145-2, preferably consisting of SEQ ID NO: 11 751.

In a particular embodiment, the invention relates to the use as defined above, wherein said fragment is the miPEP//145-2 consisting of SEQ ID NO: 11 751.

In a particular embodiment, the invention relates to the use as defined above, wherein said fragment is a fragment of miPEP 125a-1, preferably consisting of SEQ ID NO: 11 752.

In a particular embodiment, the invention relates to the use as defined above, wherein said fragment is the miPEP//125a-1 consisting of SEQ ID NO: 11 752.

In a particular embodiment, the invention relates to the use as defined above, wherein said fragment is a fragment of miPEP 15a-16-1, preferably consisting of SEQ ID NO: 11 753.

In a particular embodiment, the invention relates to the use as defined above, wherein said fragment is the miPEP//15a-16-1 consisting of SEQ ID NO: 11 753.

In a particular embodiment, the invention relates to the use as defined above, wherein said miPEP fragment is selected from:

miPEP//145-2-TAT consisting of SEQ ID NO: 11 755
(MVGLNPPLWQYGRKKRRQRRR),
miPEP//125a-1-TAT consisting of SEQ ID NO: 11 756
(MSLCLSPSLTYGRKKRRQRRR),
and
miPEP//15a-16-1-TAT consisting of SEQ ID NO: 11 757
(MFKHRFFYMHYGRKKRRQRRR).

In another aspect, the invention relates to the in vivo use of a miPEP, or a fragment of said miPEP, to induce or promote bone formation.

In another aspect, the invention relates to the in vivo use of a miPEP, or a fragment of said miPEP, to form a mineralized bone matrix.

In another aspect, the invention relates to the in vivo use of a miPEP, or a fragment of said miPEP, to promote bone formation with the help of a bone biomaterial.

Another aspect of the invention relates to a nucleic acid encoding a miPEP as defined above, or a fragment of said miPEP.

In a particular embodiment, the invention relates to a nucleic acid as defined above, said nucleic acid being selected from the group consisting of SEQ ID NO: 2q, q varying from 1 to 5 875.

In another aspect, the invention relates to an antibody specifically recognizing a miPEP selected from the group consisting of SEQ ID NO: 2q-1, q varying from 1 to 5 875, or a fragment of said miPEP.

Such antibody can be obtained from a method known to those skilled in the art, such as for example by injecting said miPEP with a non-human animal to trigger an immunization reaction and the production of antibodies by said animal.

In another aspect, the invention relates to an antibody specifically recognizing a miPEP, or a fragment of said miPEP, for its use as a drug.

In a particular embodiment, the invention relates to an antibody specifically recognizing a miPEP selected from the group consisting of SEQ ID NO: 2q-1, q varying from 1 to 5 875, or a fragment of said miPEP, for its use as a drug.

In another aspect, the invention relates to an antibody specifically recognizing a miPEP, or a fragment of said miPEP, for immunostaining said miPEP or a fragment of said miPEP.

In a particular embodiment, the invention relates to the use of an antibody as defined above, wherein said specifically recognizing a miPEP selected from the group consisting of SEQ ID NO: 2q-1, q varying from 1 to 5 875, or a fragment of said miPEP.

According to a particular embodiment, the invention relates to a miPEP from 3 to 500 amino acids encoded by a nucleotide sequence contained in the primary transcript of a miR, or a fragment of said miPEP, said miPEP being capable of modulating the accumulation of said miR in a eukaryotic cell,

for its use as a drug in the treatment of musculoskeletal disorders, or disease or trauma of bone tissue or cartilage tissue, said primary transcript of a miR being selected from the group consisting of:

let-7b, let-7g, miR-15a, miR16-1, miR106a, miR10a, miR10b, miR1246, miR125a-1, miR132, miR134, miR142, miR145, miR145-2, miR146a, miR150, miR155, miR181c, miR182, miR183, miR184, miR18b, miR192, miR195, miR200a, miR202, miR203, miR205, miR206, miR20b, miR21, miR210, miR27a, miR29a, miR29c, miR30b, miR345, miR377, miR409, miR495, miR520h, miR542, miR572, miR648, miR96 and miR99b;

preferably among the group consisting of: miR15a, miR16-1, miR125a-1 and miR145-2.

According to another particular embodiment, the invention relates to a miPEP as defined above having in particular a size from 3 to 50 amino acids, from 50 to 100 amino acids, from 100 to 500 amino acids, from 101 to 500 amino acids, from 200 to 500 amino acids, from 300 to 500 amino acids, from 400 to 500 amino acids, from 100 to 200 amino acids, from 200 to 300 amino acids or from 300 to 400 amino acids.

According to another particular embodiment, the invention relates to a miPEP, or a fragment of said miPEP, for its use as defined above, said miPEP being identified, or as identified, by implementing of a process for identifying a miPEP modulating the accumulation of a miR involved in a musculoskeletal disorder, or disease or trauma of bone tissue or cartilage tissue, comprising:
a) a step of detecting an open reading frame from 12 to 1 503 nucleotides contained in the sequence of the primary transcript of said miR, then
b) a step of comparison between:
   i. the accumulation of said miR in a specified eukaryotic cell expressing the primary transcript of said miR, in the presence of a peptide encoded by a nucleotide sequence that is identical or degenerate relative to that of said open reading frame, said peptide being present in the cell independently of transcription of the primary transcript of said miR, and
   ii. the accumulation of said miR in a eukaryotic cell of the same type as the aforesaid specified eukaryotic cell expressing the primary transcript of said miR, in the absence of said peptide,
      in which a modulation of the accumulation of said miR in the presence of said peptide relative to the accumulation of said miR in the absence of said peptide indicates the existence of a miPEP, the latter being encoded by said open reading frame and being capable of modulating the accumulation of said miR involved in a musculoskeletal disorder, or disease or trauma of bone tissue or cartilage tissue.

According to another particular embodiment, the invention relates to a miPEP, or a fragment of said miPEP, for its use as defined above, said miPEP being selected from the group of miPEPs consisting of SEQ ID NO: 2q-1,

q varying
from 45 to 48, from 89 to 92, from 101 to 104, from 121 to 124, from 133 to 140, from 153 to 156, from 161 to 164, from 169 to 192, from 221 to 224, from 233 to 256, from 301 to 304, from 317 to 320, from 329 to 332, from 337 to 340, from 381 to 384, from 445 to 448, from 469 to 472, from 533 to 536, from 693 to 696, from 789 to 792, from 909 to 916, from 977 to 980, from 1 021 to 1 024, from 1 237 to 1 240, from 1 277 to 1 280, from 1 329 to 1 332, from 1 337 to 1 344, from 1 350 to 1 353, from 1 361 to 1 364, from 1 374 to 1 377, from 1 385 to 1 388, from 1 393 to 1 404, from 1 445 to 1 448, from 1 453 to 1 456, from 1 469 to 1 472, from 1 533 to 1 536, from 1 549 to 1 552, from 1 557 to 1 560, from 1 581 to 1 584, from 1 645 to 1 652, from 1 677 to 1 680, from 1 705 to 1 716, from 1 769 to 1 772, from 1 877 to 1 880, from 1 909 to 1912, from 1 953 to 1 956, from 1 965 to 1 972, from 2 061 to 2 064, from 2 073 to 2 076, from 2 117 to 2 120, from 2 129 to 2 140, from 2 185 to 2 188, from 2 217 to 2 244, from 2 269 to 2 288, from 2 317 to 2 320, from 2 337 to 2 340, from 2 361 to 2 364, from 2 413 to 2 416, from 2 425 to 2 432, from 2 437 to 2 444, from 2 465 to 2 476, from 2 609 to 2 612, from 2 641 to 2 644, from 2 653 to 2 656, from 2 689 to 2 692, from 2 825 to 2 828, from 2 949 to 2 952, from 2 977 to 2 980, from 3 141 to 3 144, from 3 317 to 3 320, from 3 373 to 3 376, from 3 517 to 3 520, from 3 861 to 3 868, from 4 041 to 4 044, from 4 061 to 4 064, from 4 093 to 4 096, from 4 113 to 4 116, from 4 121 to 4 128, from 4 157 to 4 164, from 4 189 to 4 192, from 4 205 to 4 208, from 4 225 to 4 228, from 4 242 to 4 245, from 4 269 to 4 276, from 4 309 to 4 312, from 4 341 to 4 344, from 4 421 to 4 424, from 4 637 to 4 640, from 4 661 to 4 664, from 4 677 to 4 680, from 4 685 to 4 692, from 4 705 to 4 708, from 4 749 to 4 752, from 4 757 to 4 764, from 4 777 to 4 780, from 4 793 to 4 796, from 4 813 to 4 816, from 4 825 to 4 828, from 4 833 to 4 840, from 4 898 to 4 901, from 4 985 to 4 988, from 5 157 to 5 160, from 5 189 to 5 196, from 5 221 to 5 224, from 5 241 to 5 244, from 5 249 to 5 300, from 5 341 to 5 344, from 5 357 to 5 360, from 5 381 to 5 384, from 5 397 to 5 408, from 5 437 to 5 440, from 5 473 to 5 476, from 5 563 to 5 570, from 5 717 to 5 720, from 5 765 to 5 768, from 5 793 to 5 800, from 5 809 to 5 812, from 5 826 to 5 829, from 5 833 to 5 836 and from 5 869 to 5 875; preferably selected from the group consisting of: SEQ ID NO: 11 745, SEQ ID NO: 11 747 and SEQ ID NO: 11 749.

According to another particular embodiment, the invention relates to a miPEP, or a fragment of said miPEP, for its use as defined above for its use in the treatment of one or more musculoskeletal disorders, or diseases or traumas of bone tissue or cartilage tissue, selected from the group comprising:

arthritis, craniosynostosis, fibrous dysplasia, multiple hereditary exostoses, cleft palates, fibrodysplasia ossificans progressiva, non-ossifying fibroma, bone fracture, hyperostosis, infantile cortical hyperostosis, hyperostosis porotid, hyperparathyroidism, hypophosphatasia, Kienbôck's disease, Kôhler-Mouchet's disease, Paget's disease, Panner's disease, Scheuermann's disease, Osgood-Schlatter's disease or tibial osteochondrosis, melorheostosis, multiple myeloma, osteitis, osteitis condensas, osteitis fibrosa cystica or osteitis fibrosa or von Recklinghausen's disease, osteoarthritis, deforming osteochondritis of the hip, osteochondritis dissecans, osteochondroma, osteogenesis imperfecta or glass bone disease, osteolysis, osteomalacia, osteomyelitis, osteopenia, osteopetrosis, osteophyte, osteoporosis, osteosclerosis, pseudarthrosis, pseudarthritis pink (or non-consolidation or delayed consolidation), pycnodysostosis, Coffin-Lowry syndrome, Hajdu- Cheney syndrome, Klippel-Feil syndrome, giant cell tumor and bone tumor.

According to another particular embodiment, the invention relates to a miPEP, or a fragment of said miPEP, for its use as defined above, said miPEP being selected from the group of miPEPs consisting of: SEQ ID NO: 2q-1,

q varying from 45 to 48, from 89 to 92, from 101 to 104, from 121 to 124, from 133 to 140, from 153 to 156, from 161 to 164, from 169 to 192, from 221 to 224, from 233 to 256, from 301 to 304, from 317 to 320, from 329 to 332, from 337 to 340, from 381 to 384, from 445 to 448, from 469 to 472, from 533 to 536, from 693 to 696, from 789 to 792, from 909 to 916, from 977 to 980, from 1 021 to 1 024, from 1 237 to 1 240, from 1 277 to 1 280, from 1 329 to 1 332, from 1 337 to 1 344, from 1 350 to 1 353, from 1 361 to 1 364, from 1 374 to 1 377, from 1 385 to 1 388, from 1 393 to 1 404, from 1 445 to 1 448, from 1 453 to 1 456, from 1 469 to 1 472, from 1 533 to 1 536, from 1 549 to 1 552, from 1 557 to 1 560, from 1 581 to 1 584, from 1 645 to 1 652, from 1 677 to 1 680, from 1 705 to 1 716, from 1 769 to 1 772, from 1 877 to 1 880, from 1 909 to 1 912, from 1 953 to 1 956, from 1 965 to 1 972, from 2 061 to 2 064, from 2 073 to 2 076, from 2 117 to 2 120, from 2 129 to 2 140, from 2 185 to 2 188, from 2 217 to 2 244, from 2 269 to 2 288, from 2 317 to 2 320, from 2 337 to 2 340, from 2 361 to 2 364, from 2 413 to 2 416, from 2 425 to 2 432, from 2 437 to 2 444, from 2 465 to 2 476, from 2 609 to 2 612, from 2 641 to 2 644, from 2 653 to 2 656, from 2 689 to 2 692, from 2 825 to 2 828, from 2 949 to 2 952, from 2 977 to 2 980, from 3 141 to 3 144, from 3 317 to 3 320, from 3 373 to 3 376, from 3 517 to 3 520, from 3 861 to 3 868, from 4 041 to 4 044, from 4 061 to 4 064, from 4 093 to 4 096, from 4 113 to 4 116, from 4 121 to 4 128, from 4 157 to 4 164, from 4 189 to 4 192, from 4 205 to 4 208, from 4 225 to 4 228, from 4 242 to 4 245, from 4 269 to 4 276, from 4 309 to 4 312, from 4 341 to 4 344, from 4 421 to 4 424, from 4 637 to 4 640, from 4 661 to 4 664, from 4 677 to 4 680, from 4 685 to 4 692, from 4 705 to 4 708, from 4 749 to 4 752, from 4 757 to 4 764, from 4 777 to 4 780, from 4 793 to 4 796, from 4 813 to 4 816, from 4 825 to 4 828, from 4 833 to 4 840, from 4 898 to 4 901, from 4 985 to 4 988, from 5 157 to 5 160, from 5 189 to 5 196, from 5 221 to 5 224, from 5 241 to 5 244, from 5 249 to 5 300, from 5 341 to 5 344, from 5 357 to 5 360, from 5 381 to 5 384, from 5 397 to 5 408, from 5 437 to 5 440, from 5 473 to 5 476, from 5 563 to 5 570, from 5 717 to 5 720, from 5 765 to 5 768, from 5 793 to 5 800, from 5 809 to 5 812, from 5 826 to 5 829, from 5 833 to 5 836 and from 5 869 to 5 875;
preferably selected from the group consisting of: SEQ ID NO: 11 745, SEQ ID NO: 11 747 and SEQ ID NO: 11 749.

According to another particular embodiment, the invention relates to a miPEP fragment for its use as defined above, said miPEP fragment being selected from:
miPEP//145-2 consisting of SEQ ID NO: 11 751,
miPEP//125a-1 consisting of SEQ ID NO: 11 752, and
miPEP//15a-16-1 consisting of SEQ ID NO: 11 753.

According to another particular embodiment, the invention relates to a miPEP, or a fragment of said miPEP, for its use as defined above, said miPEP, or said fragment of said miPEP, being fused to or linked to one or more molecules facilitating the entry into the cell of the miPEP or miPEP fragment,

said miPEP, or fragment of said miPEP, being preferably fused in N-ter or in C-ter to the TAT peptide (YGRKKRRQRRR, SEQ ID NO: 11 754).

According to another particular embodiment, the invention relates to a miPEP selected from the group of miPEPs consisting of: SEQ ID NO: 2q-1, q varying from 45 to 48, from 89 to 92, from 101 to 104, from 121 to 124, from 133 to 140, from 153 to 156, from 161 to 164, from 169 to 192, from 221 to 224, from 233 to 256, from 301 to 304, from 317 to 320, from 329 to 332, from 337 to 340, from 381 to 384, from 445 to 448, from 469 to 472, from 533 to 536, from 693 to 696, from 789 to 792, from 909 to 916, from 977 to 980, from 1 021 to 1 024, from 1 237 to 1 240, from 1 277 to 1 280, from 1 329 to 1 332, from 1 337 to 1 344, from 1 350 to 1 353, from 1 361 to 1 364, from 1 374 to 1 377, from 1 385 to 1 388, from 1 393 to 1 404, from 1 445 to 1448, from 1 453 to 1 456, from 1 469 to 1 472, from 1 533 to 1 536, from 1 549 to 1 552, from 1 557 to 1 560, from 1 581 to 1 584, from 1 645 to 1 652, from 1 677 to 1 680, from 1 705 to 1 716, from 1 769 to 1 772, from 1 877 to 1 880, from 1 909 to 1 912, from 1 953 to 1956, from 1 965 to 1 972, from 2 061 to 2 064, from 2 073 to 2 076, from 2 117 to 2 120, from 2 129 to 2 140, from 2 185 to 2 188, from 2 217 to 2 244, from 2 269 to 2 288, from 2 317 to 2 320, from 2 337 to 2 340, from 2 361 to 2 364, from 2 413 to 2 416, from 2 425 to 2 432, from 2 437 to 2 444, from 2 465 to 2 476, from 2 609 to 2 612, from 2 641 to 2 644, from 2 653 to 2 656, from 2 689 to 2 692, from 2 825 to 2 828, from 2 949 to 2 952, from 2 977 to 2 980, from 3 141 to 3 144, from 3 317 to 3 320, from 3 373 to 3 376, from 3 517 to 3 520, from 3 861 to 3 868, from 4 041 to 4 044, from 4 061 to 4 064, from 4 093 to 4 096, from 4 113 to 4 116, from 4 121 to 4 128, from 4 157 to 4 164, from 4 189 to 4 192, from 4 205 to 4 208, from 4 225 to 4 228, from 4 242 to 4 245, from 4 269 to 4 276, from 4 309 to 4 312, from 4 341 to 4 344, from 4 421 to 4 424, from 4 637 to 4 640, from 4 661 to 4 664, from 4 677 to 4 680, from 4 685 to 4 692, from 4 705 to 4 708, from 4 749 to 4 752, from 4 757 to 4 764, from 4 777 to 4 780, from 4 793 to 4 796, from 4 813 to 4 816, from 4 825 to 4 828, from 4 833 to 4 840, from 4 898 to 4 901, from 4 985 to 4 988, from 5 157 to 5 160, from 5 189 to 5 196, from 5 221 to 5 224, from 5 241 to 5 244, from 5 249 to 5 300, from 5 341 to 5 344, from 5 357 to 5 360, from 5 381 to 5 384, from 5 397 to 5 408, from 5 437 to 5 440, from 5 473 to 5 476, from 5 563 to 5 570, from 5 717 to 5 720, from 5 765 to 5 768, from 5 793 to 5 800, from 5 809 to 5 812, from 5 826 to 5 829, from 5 833 to 5 836 and from 5 869 to 5 875;

preferably selected from the group consisting of: SEQ ID NO: 11 745, SEQ ID NO: 11 747 and SEQ ID NO: 11 749,
said miPEP, or fragment of said miPEP, being preferably fused to the TAT peptide (YGRKKRRQRRR, SEQ ID NO: 11 754), fused to penetratin, fused to a polyhistidine peptide, fused to a polyarginine peptide, or linked to one or more palmitic acid molecules.

According to another particular embodiment, the invention relates to a nucleic acid encoding one of the miPEPs selected from the group of miPEPs consisting of SEQ ID NO: 2q, q varying from 45 to 48, from 89 to 92, from 101 to 104, from 121 to 124, from 133 to 140, from 153 to 156, from 161 to 164, from 169 to 192, from 221 to 224, from 233 to 256, from 301 to 304, from 317 to 320, from 329 to 332, from 337 to 340, from 381 to 384, from 445 to 448, from 469 to 472, from 533 to 536, from 693 to 696, from 789 to 792, from 909 to 916, from 977 to 980, from 1 021 to 1 024, from 1 237 to 1 240, from 1 277 to 1 280, from 1 329 to 1 332, from 1 337 to 1 344, from 1 350 to 1 353, from 1 361 to 1 364, from 1 374 to 1 377, from 1 385 to 1 388, from 1 393 to 1 404, from 1 445 to 1 448, from 1 453 to 1 456, from 1 469 to 1 472, from 1 533 to 1 536, from 1 549 to 1 552, from 1 557 to 1 560, from 1 581 to 1 584, from 1 645 to 1 652, from 1 677 to 1 680, from 1 705 to 1 716, from 1 769 to 1 772, from 1 877 to 1 880, from 1 909 to 1912, from 1 953 to 1 956, from 1 965 to 1 972, from 2 061 to 2 064, from 2 073 to 2 076, from 2 117 to 2 120, from 2 129 to 2 140, from 2 185 to 2 188, from 2 217 to 2 244, from 2 269 to 2 288, from 2 317 to 2 320, from 2 337 to 2 340, from 2 361 to 2 364, from 2 413 to 2 416, from 2 425 to 2 432, from 2 437 to 2 444, from 2 465 to 2 476, from 2 609 to 2 612, from 2 641 to 2 644, from 2 653 to 2 656, from 2 689 to 2 692, from 2 825 to 2 828, from 2 949 to 2 952, from 2 977 to 2 980, from 3 141 to 3 144, from 3 317 to 3 320, from 3 373 to 3 376, from 3 517 to 3 520, from 3 861 to 3 868, from 4 041 to 4 044, from 4 061 to 4 064, from 4 093 to 4 096, from 4 113 to 4 116, from 4 121 to 4 128, from 4 157 to 4 164, from 4 189 to 4 192, from 4 205 to 4 208, from 4 225 to 4 228, from 4 242 to 4 245, from 4 269 to 4 276, from 4 309 to 4 312, from 4 341 to 4 344, from 4 421 to 4 424, from 4 637 to 4 640, from 4 661 to 4 664, from 4 677 to 4 680, from 4 685 to 4 692, from 4 705 to 4 708, from 4 749 to 4 752, from 4 757 to 4 764, from 4 777 to 4 780, from 4 793 to 4 796, from 4 813 to 4 816, from 4 825 to 4 828, from 4 833 to 4 840, from 4 898 to 4 901, from 4 985 to 4 988, from 5 157 to 5 160, from 5 189 to 5 196, from 5 221 to 5 224, from 5 241 to 5 244, from 5 249 to 5 300, from 5 341 to 5 344, from 5 357 to 5 360, from 5 381 to 5 384, from 5 397 to 5 408, from 5 437 to 5 440, from 5 473 to 5 476, from 5 563 to 5 570, from 5 717 to 5 720, from 5 765 to 5 768, from 5 793 to 5 800, from 5 809 to 5 812, from 5 826 to 5 829, from 5 833 to 5 836 and from 5 869 to 5 875; preferably selected from the group consisting of: SEQ ID NO: 11 746, SEQ ID NO: 11 748 and SEQ ID NO: 11 750.

Furthermore, the invention also relates to a process for identifying a miPEP modulating the accumulation of a miR involved in a musculoskeletal disorder, or disease or trauma of bone tissue or cartilage tissue, said miPEP being encoded by a nucleotide sequence contained in the primary transcript of a miR, comprising:
a) a step of detecting an open reading frame from 12 to 1 503 nucleotides contained in the sequence of the primary transcript of said miR, then
b) a step of comparison between:
   i. the accumulation of said miR in a specified eukaryotic cell expressing the primary transcript of said miR, in the presence of a peptide encoded by a nucleotide sequence that is identical or degenerate relative to that of said open reading frame, said peptide being present in the cell independently of transcription of the primary transcript of said miR, and
   ii. the accumulation of said miR in a eukaryotic cell of the same type as the aforesaid specified eukaryotic cell expressing the primary transcript of said miR, in the absence of said peptide,
      in which a modulation of the accumulation of said miR in the presence of said peptide relative to the accumulation of said miR in the absence of said peptide indicates the existence of a miPEP, the latter being encoded by said open reading frame and being capable of modulating the accumulation of said miR involved in a musculoskeletal disorder, or disease or trauma of bone tissue or cartilage tissue.

According to another aspect, the invention also relates to the in vitro, in vivo or ex vivo use of a miPEP or a fragment of said miPEP,

said miPEP being selected from the group of miPEPs consisting of: SEQ ID NO: 2q-1,
q varying
from 45 to 48, from 89 to 92, from 101 to 104, from 121 to 124, from 133 to 140, from 153 to 156, from 161 to 164, from 169 to 192, from 221 to 224, from 233 to 256, from 301 to 304, from 317 to 320, from 329 to 332, from 337 to 340, from 381 to 384, from 445 to 448, from 469 to 472, from 533 to 536, from 693 to 696, from 789 to 792, from 909 to 916, from 977 to 980, from 1 021 to 1 024, from 1 237 to 1 240, from 1 277 to 1 280, from 1 329 to 1 332, from 1 337 to 1 344, from 1 350 to 1 353, from 1 361 to 1 364, from 1 374 to 1 377, from 1 385 to 1 388, from 1 393 to 1 404, from 1 445 to 1 448, from 1 453 to 1 456, from 1 469 to 1 472, from 1 533 to 1 536, from 1 549 to 1 552, from 1 557 to 1 560, from 1 581 to 1 584, from 1 645 to 1 652, from 1 677 to 1 680, from 1 705 to 1 716, from 1 769 to 1 772, from 1 877 to 1 880, from 1 909 to 1 912, from 1 953 to 1 956, from 1 965 to 1 972, from 2 061 to 2 064, from 2 073 to 2 076, from 2 117 to 2 120, from 2 129 to 2 140, from 2 185 to 2 188, from 2 217 to 2 244, from 2 269 to 2 288, from 2 317 to 2 320, from 2 337 to 2 340, from 2 361 to 2 364, from 2 413 to 2 416, from 2 425 to 2 432, from 2 437 to 2 444, from 2 465 to 2 476, from 2 609 to 2 612, from 2 641 to 2 644, from 2 653 to 2 656, from 2 689 to 2 692, from 2 825 to 2 828, from 2 949 to 2 952, from 2 977 to 2 980, from 3 141 to 3 144, from 3 317 to 3 320, from 3 373 to 3 376, from 3 517 to 3 520, from 3 861 to 3 868, from 4 041 to 4 044, from 4 061 to 4 064, from 4 093 to 4 096, from 4 113 to 4 116, from 4 121 to 4 128, from 4 157 to 4 164, from 4 189 to 4 192, from 4 205 to 4 208, from 4 225 to 4 228, from 4 242 to 4 245, from 4 269 to 4 276, from 4 309 to 4 312, from 4 341 to 4 344, from 4 421 to 4 424, from 4 637 to 4 640, from 4 661 to 4 664, from 4 677 to 4 680, from 4 685 to 4 692, from 4 705 to 4 708, from 4 749 to 4 752, from 4 757 to 4 764, from 4 777 to 4 780, from 4 793 to 4 796, from 4 813 to 4 816, from 4 825 to 4 828, from 4 833 to 4 840, from 4 898 to 4 901, from 4 985 to 4 988, from 5 157 to 5 160, from 5 189 to 5 196, from 5 221 to 5 224, from 5 241 to 5 244, from 5 249 to 5 300, from 5 341 to 5 344, from 5 357 to 5 360, from 5 381 to 5 384, from 5 397 to 5 408, from 5 437 to 5 440, from 5 473 to 5 476, from 5 563 to 5 570, from 5 717 to 5 720, from 5 765 to 5 768, from 5 793 to 5 800, from 5 809 to 5 812, from 5 826 to 5 829, from 5 833 to 5 836 and from 5 869 to 5 875; preferably selected from the group consisting of: SEQ ID NO: 11 745, SEQ ID NO: 11 747 and SEQ ID NO: 11 749,
to promote osteogenesis of cells, particularly mesenchymal stem cells.

According to another particular aspect, the invention relates to the use of a miPEP or fragment of said miPEP as defined above, in combination with one or more additives, said additives being selected from the group consisting of: cytokines such as bone morphogenetic protein (BMP) and/or dexamethasone.

According to another particular aspect, the invention relates to a use of a miPEP or fragment of said miPEP as defined above, in combination with a biomaterial, said biomaterial being selected from the following table:

TABLE 1
List of possible biomaterials for implementing the invention.

| a) natural | b) synthetic | c) bioactive |
|---|---|---|
| polymers | polymers | glasses |
| gelatin/chitooligosaccharide, | poly(lactide-co-glycolide) | 45S5 Bioglass ® |
| collagen, | [PLGA], | (registered |
| chitosan, | poly(ε-caprolactone) [PCL] or | trademark of the |
| chitosan/collagen/beta- | poly(hydroxymethylglycolide- | University of |
| glycerophosphate (β-GP), | co-ε-caprolactone) [pHMGCL], | Florida), |
| silk fibroins, | PLGA-poly(ethylene oxide) | CaMgSi₂O₆, |
| alginate/calcium phosphate | (PEO) | 45S5 bioactive |
| cement (CPC)[=alginate/CPC] | [=PLGA-PEO], | glass ® (registered |
| alginate, | poly(L-lactide-co-ε- | trademark of the |
| hyaluronic acid, | caprolactone)/poly(L-lactide-co- | University of |
| RAD16-I BD ™ (PuraMatrix ™, | 1,5-dioxepan-2-one)[poly(LLA- | Florida), |
| registered trademark of 3-D | co-CL)/poly(LLA-co-DXO)], | 45S bioactive |
| Matrix, Ltd.), or | poly[(ethylglycinato)(p- | glass ®, |
| chitosan/peptide “Arginine- | methylphenoxy) | bioactive glass ® |
| Glycine-Aspartic acid” (RGD) | phosphazene]/PLGA | (BG20), |
| [=chitosan/peptide RGD] | [=PPHOS/PLGA], | bioglass ®, or |
| | PCL/poly(diisopropyl fumarate) | bioactive glass ® |
| | (PDIPF) | (13-93) |
| | [=PCL/PDIPF], | |
| | poly(ester amide)-g-TA [PEA-g- | |
| | TAJ, | |
| | PLGA-poly(ethylene glycol- | |
| | aspartic acid (PEG-ASP) [= | |
| | PLGA-(PEG-ASP)], | |
| | poly(ethylene oxide | |
| | terephthalate)/poly(butylene | |
| | terephthalate) [PEOT/PBT], or | |
| | poly (hydroxyethyl | |
| | methacrylate) [poly-HEMA] | |

| d) calcium phosphates | e) coral | f) metals |
|---|---|---|
| CPC, | Porites sp., | NiTi/Ti, |
| hydroxyapatite [HA] | Goniopora coral, | titanium (Ti) fiber |
| β-tricalcium phosphate [βTCP] | hydroxyapatite coral, | sheets, |
| HA/βTCP, | coralline in the shape of a | titanium wire, |
| calcium Aluminate/melatonine, | condyle, | Ti, |
| or | Porites lutea, | TiO₂, |
| CPC/Bioglass ® | Porites acropora, or | Ti₆Ta₄Sn alloy, |
| | natural coral matrices | Magnesium W4 |
| | | alloy (MgY4), or |
| | | Iron-Manganese |
| | | alloy |

| g) polymer/ceramic | h) metal/ceramic |
|---|---|
| composites | composites |
| Nano-apatite/PCL, | polyNaSS/Ti, |
| PLGA-PCL-Calcium Phosphate | polylactic acid in crystalline |
| (CP) | form/PLGA/Ti |
| [=PLGA-PCL-CP], | [=PLLA/PLGA/Ti], |
| PEOT/PBT-CP, | polyNaSS/Ti, |
| Collagen-CPC, | NaSS/methacrylic acid, |
| PCL-TCP, | MA/Ti₆Al₄V alloy, |
| Chitosan-CPC, | polyNaSS/Ti, |
| Magnesium Phosphate-PCL, | SiO₂/CaO/Na₂O/MgO/B₂O₃, |
| PLGA-βTCP, | Niobium/fluorapatite, |
| PCLF-PVA-HA (PVA: | Ti-HA, |
| poly(alcool vinylic)), | TiO₂, |
| Chitosan/poly(DL, lactide-co- | ZrO₂—CP/PCL, |
| glycolide), | K/Sr-calcium polyphosphate |
| PLGA-nHA, ou | (CPP) |
| poly-3-hydroxybutyrate-HA | [=K/Sr-CPP], |
| (P3HB-HA) | ZrO₂/HA, |
| | porous material containing |
| | cobalt and a mesoporous |
| | Bioglass ® [Co-MBG], |
| | porous material containing |
| | copper and a mesoporous |
| | Bioglass ® [Cu-MBG], |
| | β-TCP/Mg, or |
| | Sr/CPP |

According to another particular aspect, the invention relates to the use of a miPEP or fragment of said miPEP as defined above, in combination with one or more additives, said additives being chosen from the group consisting of: cytokines as well as bone morphogenetic protein (BMP) and/or dexamethasone;

and in combination with a biomaterial, said biomaterial being selected from the group shown in Table 1 (cf p39-41).

According to another particular aspect, the invention relates to the use of a miPEP fragment of sequence SEQ ID NO: 11 752 to promote osteogenesis of mesenchymal stem cells in combination with a biomaterial, said biomaterial being the HA/β-TCP in a ratio of 20/80.

Another aspect of the invention is that it relates to osteo-induced cells with one or more miPEP or fragment of said miPEP as defined above, said cells being in particular mesenchymal stem cells.

Another aspect of the invention is that it relates to cells comprising a nucleic acid encoding one or more of the miPEPs as defined above, the expression of said nucleic acid being inducible or not, said cells being in particular mesenchymal stem cells.

According to another particular aspect, the invention relates to a composite biomaterial comprising osteo-induced cells as defined above or cells as defined above, in combination or not with one or more additives, said additives being chosen from the group consisting of: cytokines such as bone morphogenetic proteins (BMP) and/or dexamethasone, said biomaterial being selected from the group shown in Table 1 (cf. p.39-41).

According to another particular aspect, the invention relates to a composite biomaterial comprising osteo-induced mesenchymal stem cells with a miPEP fragment of sequence SEQ ID NO: 11 752, said biomaterial being the HA/β-TCP in a ratio of 20/80.

TABLE 2
MiRs involved in cancer, bacterial or fungal infections, cardiovascular
diseases, hereditary congenital diseases, skin diseases and eye diseases

| | Bacterial or | | Hereditary | | |
|---|---|---|---|---|---|
| | fungal | Cardiovascular | congenital | Skin | Eye |
| Cancer | infections | diseases | diseases | diseases | diseases |
| hsa-let-7a-2 | hsa-let-7a-2 | hsa-let-7a-2 | hsa-let-7a-2 | hsa-let-7b | hsa-let-7a-2 |
| hsa-let-7a-3 | hsa-let-7a-3 | hsa-let-7a-3 | hsa-let-7a-3 | hsa-let-7g | hsa-let-7a-3 |
| hsa-let-7b | hsa-let-7b | hsa-let-7b | hsa-let-7b | hsa-let-7i | hsa-let-7b |
| hsa-let-7d | hsa-let-7d | hsa-let-7d | hsa-let-7d | hsa-mir-106a | hsa-let-7d |
| hsa-let-7e | hsa-let-7e | hsa-let-7e | hsa-let-7e | hsa-mir-10a | hsa-let-7e |
| hsa-let-7g | hsa-let-7g | hsa-let-7g | hsa-let-7g | hsa-mir-10b | hsa-let-7g |
| hsa-let-7i | hsa-let-7i | hsa-let-7i | hsa-let-7i | hsa-mir-1246 | hsa-let-7i |
| hsa-mir-100 | hsa-mir-100 | hsa-mir-100 | hsa-mir-100 | hsa-mir-132 | hsa-mir-106a |
| hsa-mir-106a | hsa-mir-106a | hsa-mir-106a | hsa-mir-106a | hsa-mir-134 | hsa-mir-10a |
| hsa-mir-10a | hsa-mir-10a | hsa-mir-10a | hsa-mir-10a | hsa-mir-141 | hsa-mir-10b |
| hsa-mir-10b | hsa-mir-10b | hsa-mir-10b | hsa-mir-10b | hsa-mir-142 | hsa-mir-1246 |
| hsa-mir-122 | hsa-mir-122 | hsa-mir-122 | hsa-mir-122 | hsa-mir-145 | hsa-mir-125a |
| hsa-mir-1246 | hsa-mir-1246 | hsa-mir-1246 | hsa-mir-1246 | hsa-mir-146a | hsa-mir-134 |
| hsa-mir-125a | hsa-mir-129-1 | hsa-mir-125a | hsa-mir-125a | hsa-mir-148a | hsa-mir-141 |
| hsa-mir-130b | hsa-mir-130b | hsa-mir-129-1 | hsa-mir-130b | hsa-mir-150 | hsa-mir-143 |
| hsa-mir-132 | hsa-mir-133b | hsa-mir-130b | hsa-mir-132 | hsa-mir-155 | hsa-mir-144 |
| hsa-mir-133b | hsa-mir-134 | hsa-mir-132 | hsa-mir-134 | hsa-mir-181c | hsa-mir-145 |
| hsa-mir-134 | hsa-mir-141 | hsa-mir-133b | hsa-mir-141 | hsa-mir-182 | hsa-mir-155 |
| hsa-mir-141 | hsa-mir-142 | hsa-mir-134 | hsa-mir-142 | hsa-mir-183 | hsa-mir-181c |
| hsa-mir-142 | hsa-mir-143 | hsa-mir-141 | hsa-mir-143 | hsa-mir-184 | hsa-mir-182 |
| hsa-mir-143 | hsa-mir-144 | hsa-mir-142 | hsa-mir-145 | hsa-mir-18b | hsa-mir-183 |
| hsa-mir-144 | hsa-mir-145 | hsa-mir-143 | hsa-mir-146a | hsa-mir-192 | hsa-mir-18b |
| hsa-mir-145 | hsa-mir-146a | hsa-mir-144 | hsa-mir-146b | hsa-mir-195 | hsa-mir-192 |
| hsa-mir-146a | hsa-mir-154 | hsa-mir-145 | hsa-mir-148a | hsa-mir-200a | hsa-mir-195 |
| hsa-mir-146b | hsa-mir-155 | hsa-mir-146a | hsa-mir-154 | hsa-mir-202 | hsa-mir-200b |
| hsa-mir-148a | hsa-mir-181a-1 | hsa-mir-146b | hsa-mir-155 | hsa-mir-203 | hsa-mir-202 |
| hsa-mir-150 | hsa-mir-181a-2 | hsa-mir-150 | hsa-mir-181a-2 | hsa-mir-205 | hsa-mir-203 |
| hsa-mir-154 | hsa-mir-181c | hsa-mir-154 | hsa-mir-181c | hsa-mir-206 | hsa-mir-205 |
| hsa-mir-155 | hsa-mir-182 | hsa-mir-155 | hsa-mir-181d | hsa-mir-20b | hsa-mir-20b |
| hsa-mir-181c | hsa-mir-183 | hsa-mir-181a-1 | hsa-mir-182 | hsa-mir-21 | hsa-mir-21 |
| hsa-mir-181d | hsa-mir-184 | hsa-mir-181a-2 | hsa-mir-183 | hsa-mir-210 | hsa-mir-210 |
| hsa-mir-182 | hsa-mir-187 | hsa-mir-181c | hsa-mir-18b | hsa-mir-221 | hsa-mir-212 |
| hsa-mir-183 | hsa-mir-18b | hsa-mir-181d | hsa-mir-192 | hsa-mir-222 | hsa-mir-221 |
| hsa-mir-184 | hsa-mir-192 | hsa-mir-182 | hsa-mir-193a | hsa-mir-27a | hsa-mir-222 |
| hsa-mir-187 | hsa-mir-193b | hsa-mir-183 | hsa-mir-193b | hsa-mir-29a | hsa-mir-23a |
| hsa-mir-18b | hsa-mir-195 | hsa-mir-184 | hsa-mir-195 | hsa-mir-29c | hsa-mir-27a |
| hsa-mir-192 | hsa-mir-196a-2 | hsa-mir-187 | hsa-mir-202 | hsa-mir-30a | hsa-mir-298 |
| hsa-mir-193b | hsa-mir-19b-2 | hsa-mir-18b | hsa-mir-203 | hsa-mir-30b | hsa-mir-30a |
| hsa-mir-195 | hsa-mir-200b | hsa-mir-192 | hsa-mir-206 | hsa-mir-345 | hsa-mir-30d |
| hsa-mir-200a | hsa-mir-200c | hsa-mir-193a | hsa-mir-20b | hsa-mir-34a | hsa-mir-331 |
| hsa-mir-200b | hsa-mir-202 | hsa-mir-193b | hsa-mir-21 | hsa-mir-377 | hsa-mir-345 |
| hsa-mir-200c | hsa-mir-203 | hsa-mir-195 | hsa-mir-210 | hsa-mir-409 | hsa-mir-377 |
| hsa-mir-202 | hsa-mir-205 | hsa-mir-19b-2 | hsa-mir-212 | hsa-mir-495 | hsa-mir-495 |
| hsa-mir-203 | hsa-mir-206 | hsa-mir-200a | hsa-mir-217 | hsa-mir-520h | hsa-mir-542 |
| hsa-mir-205 | hsa-mir-20b | hsa-mir-200c | hsa-mir-221 | hsa-mir-542 | hsa-mir-572 |
| hsa-mir-206 | hsa-mir-21 | hsa-mir-202 | hsa-mir-222 | hsa-mir-572 | hsa-mir-648 |
| hsa-mir-20b | hsa-mir-210 | hsa-mir-203 | hsa-mir-23a | hsa-mir-648 | hsa-mir-96 |
| hsa-mir-21 | hsa-mir-2114 | hsa-mir-205 | hsa-mir-27a | hsa-mir-92b | hsa-mir-99b |
| hsa-mir-210 | hsa-mir-212 | hsa-mir-206 | hsa-mir-29a | hsa-mir-96 | |
| hsa-mir-212 | hsa-mir-217 | hsa-mir-20b | hsa-mir-29c | hsa-mir-99b | |
| hsa-mir-216a | hsa-mir-219-2 | hsa-mir-21 | hsa-mir-30a | | |
| hsa-mir-217 | hsa-mir-221 | hsa-mir-210 | hsa-mir-30b | | |
| hsa-mir-221 | hsa-mir-222 | hsa-mir-212 | hsa-mir-30c-2 | | |
| hsa-mir-222 | hsa-mir-23a | hsa-mir-217 | hsa-mir-30d | | |
| hsa-mir-23a | hsa-mir-24-2 | hsa-mir-221 | hsa-mir-3178 | | |
| hsa-mir-27a | hsa-mir-27a | hsa-mir-222 | hsa-mir-331 | | |
| hsa-mir-297 | hsa-mir-296 | hsa-mir-23a | hsa-mir-345 | | |
| hsa-mir-298 | hsa-mir-29a | hsa-mir-24-2 | hsa-mir-34a | | |
| hsa-mir-29a | hsa-mir-29b-1 | hsa-mir-27a | hsa-mir-363 | | |
| hsa-mir-29b-1 | hsa-mir-29b-2 | hsa-mir-29a | hsa-mir-369 | | |
| hsa-mir-29c | hsa-mir-29c | hsa-mir-29b-1 | hsa-mir-371 | | |
| hsa-mir-301b | hsa-mir-30a | hsa-mir-29b-2 | hsa-mir-372 | | |
| hsa-mir-30a | hsa-mir-30b | hsa-mir-29c | hsa-mir-373 | | |
| hsa-mir-30b | hsa-mir-30d | hsa-mir-30a | hsa-mir-374a | | |
| hsa-mir-30d | hsa-mir-331 | hsa-mir-30b | hsa-mir-377 | | |
| hsa-mir-320 | hsa-mir-345 | hsa-mir-30c-2 | hsa-mir-379 | | |
| hsa-mir-331 | hsa-mir-34a | hsa-mir-30d | hsa-mir-409 | | |
| hsa-mir-345 | hsa-mir-648 | hsa-mir-3178 | hsa-mir-422a | | |
| hsa-mir-34a | hsa-mir-96 | hsa-mir-331 | hsa-mir-494 | | |
| hsa-mir-373 | hsa-mir-99b | hsa-mir-345 | hsa-mir-495 | | |
| hsa-mir-374a | | hsa-mir-34a | hsa-mir-497 | | |
| hsa-mir-377 | | hsa-mir-363 | hsa-mir-505 | | |
| hsa-mir-379 | | hsa-mir-369 | hsa-mir-518c | | |
| hsa-mir-382 | | hsa-mir-371 | hsa-mir-519e | | |
| hsa-mir-409 | | hsa-mir-372 | hsa-mir-520a | | |
| hsa-mir-422a | | hsa-mir-373 | hsa-mir-520g | | |
| hsa-mir-429 | | hsa-mir-374a | hsa-mir-522 | | |
| hsa-mir-485 | | hsa-mir-377 | hsa-mir-523 | | |
| hsa-mir-493 | | hsa-mir-379 | hsa-mir-542 | | |
| hsa-mir-494 | | hsa-mir-409 | hsa-mir-572 | | |
| hsa-mir-495 | | hsa-mir-422a | hsa-mir-648 | | |
| hsa-mir-496 | | hsa-mir-494 | hsa-mir-654 | | |
| hsa-mir-497 | | hsa-mir-495 | hsa-mir-665 | | |
| hsa-mir-506 | | hsa-mir-497 | hsa-mir-769 | | |
| hsa-mir-507 | | hsa-mir-505 | hsa-mir-92b | | |
| hsa-mir-508 | | hsa-mir-518c | hsa-mir-96 | | |
| hsa-mir-509-1 | | hsa-mir-519e | hsa-mir-99b | | |
| hsa-mir-510 | | hsa-mir-520a | | | |
| hsa-mir-520h | | hsa-mir-520g | | | |
| hsa-mir-523 | | hsa-mir-520h | | | |
| hsa-mir-525 | | hsa-mir-522 | | | |
| hsa-mir-542 | | hsa-mir-523 | | | |
| hsa-mir-568 | | hsa-mir-542 | | | |
| hsa-mir-570 | | hsa-mir-572 | | | |
| hsa-mir-572 | | hsa-mir-648 | | | |
| hsa-mir-633 | | hsa-mir-654 | | | |
| hsa-mir-648 | | hsa-mir-665 | | | |
| hsa-mir-7-2 | | hsa-mir-769 | | | |
| hsa-mir-92b | | hsa-mir-92b | | | |
| hsa-mir-940 | | hsa-mir-96 | | | |
| hsa-mir-96 | | hsa-mir-99b | | | |
| hsa-mir-99b | | hsa-mir-15a | | | |
| | | hsa-mir-15b | | | |

TABLE 3
MiRs involved in diseases of the digestive system, diseases of the endocrine
system, diseases of the nervous system, diseases related to viruses, diseases
related to nutrition and metabolism and lymphatic diseases and hemopathies

| Diseases | Diseases | Diseases | Diseases | Diseases | Lymphatic |
|---|---|---|---|---|---|
| of the | of the | of the | related | related to | diseases |
| digestive | endocrine | nervous | to | nutrition and | and |
| system | system | system | viruses | metabolism | hemopathies |
| hsa-let-7a-2 | hsa-let-7a-2 | hsa-let-7b | hsa-let-7b | hsa-let-7b | hsa-let-7a-2 |
| hsa-let-7a-3 | hsa-let-7a-3 | hsa-let-7d | hsa-let-7d | hsa-let-7g | hsa-let-7a-3 |
| hsa-let-7b | hsa-let-7b | hsa-let-7g | hsa-let-7g | hsa-let-7i | hsa-let-7b |
| hsa-let-7d | hsa-let-7d | hsa-let-7i | hsa-mir-106a | hsa-mir-100 | hsa-let-7d |
| hsa-let-7e | hsa-let-7e | hsa-mir-100 | hsa-mir-10a | hsa-mir-106a | hsa-let-7e |
| hsa-let-7g | hsa-let-7g | hsa-mir-106a | hsa-mir-10b | hsa-mir-10a | hsa-let-7g |
| hsa-let-7i | hsa-let-7i | hsa-mir-10a | hsa-mir-122 | hsa-mir-10b | hsa-let-7i |
| hsa-mir-100 | hsa-mir-106a | hsa-mir-10b | hsa-mir-1246 | hsa-mir-122 | hsa-mir-106a |
| hsa-mir-106a | hsa-mir-10a | hsa-mir-122 | hsa-mir-132 | hsa-mir-1246 | hsa-mir-10a |
| hsa-mir-10a | hsa-mir-10b | hsa-mir-1246 | hsa-mir-134 | hsa-mir-125a | hsa-mir-125a |
| hsa-mir-10b | hsa-mir-1246 | hsa-mir-125a | hsa-mir-145 | hsa-mir-129-1 | hsa-mir-130b |
| hsa-mir-122 | hsa-mir-132 | hsa-mir-129-1 | hsa-mir-155 | hsa-mir-130b | hsa-mir-132 |
| hsa-mir-1246 | hsa-mir-134 | hsa-mir-130b | hsa-mir-181c | hsa-mir-132 | hsa-mir-141 |
| hsa-mir-125a | hsa-mir-142 | hsa-mir-132 | hsa-mir-182 | hsa-mir-133b | hsa-mir-142 |
| hsa-mir-130b | hsa-mir-145 | hsa-mir-133b | hsa-mir-183 | hsa-mir-134 | hsa-mir-143 |
| hsa-mir-132 | hsa-mir-150 | hsa-mir-134 | hsa-mir-18b | hsa-mir-141 | hsa-mir-144 |
| hsa-mir-134 | hsa-mir-155 | hsa-mir-141 | hsa-mir-192 | hsa-mir-142 | hsa-mir-145 |
| hsa-mir-141 | hsa-mir-181c | hsa-mir-142 | hsa-mir-195 | hsa-mir-143 | hsa-mir-146a |
| hsa-mir-143 | hsa-mir-182 | hsa-mir-143 | hsa-mir-202 | hsa-mir-144 | hsa-mir-150 |
| hsa-mir-145 | hsa-mir-183 | hsa-mir-144 | hsa-mir-206 | hsa-mir-145 | hsa-mir-155 |
| hsa-mir-146a | hsa-mir-184 | hsa-mir-145 | hsa-mir-20b | hsa-mir-146a | hsa-mir-181c |
| hsa-mir-148a | hsa-mir-18b | hsa-mir-146a | hsa-mir-21 | hsa-mir-146b | hsa-mir-182 |
| hsa-mir-150 | hsa-mir-192 | hsa-mir-146b | hsa-mir-210 | hsa-mir-154 | hsa-mir-183 |
| hsa-mir-154 | hsa-mir-195 | hsa-mir-154 | hsa-mir-212 | hsa-mir-155 | hsa-mir-187 |
| hsa-mir-155 | hsa-mir-200a | hsa-mir-155 | hsa-mir-217 | hsa-mir-181a-1 | hsa-mir-18b |
| hsa-mir-182 | hsa-mir-202 | hsa-mir-181a-1 | hsa-mir-296 | hsa-mir-181a-2 | hsa-mir-192 |
| hsa-mir-183 | hsa-mir-203 | hsa-mir-181a-2 | hsa-mir-30d | hsa-mir-181c | hsa-mir-193b |
| hsa-mir-18b | hsa-mir-205 | hsa-mir-181c | hsa-mir-345 | hsa-mir-181d | hsa-mir-195 |
| hsa-mir-192 | hsa-mir-20b | hsa-mir-181d | hsa-mir-34a | hsa-mir-182 | hsa-mir-200b |
| hsa-mir-195 | hsa-mir-21 | hsa-mir-182 | hsa-mir-648 | hsa-mir-183 | hsa-mir-202 |
| hsa-mir-200a | hsa-mir-210 | hsa-mir-183 | hsa-mir-96 | hsa-mir-184 | hsa-mir-203 |
| hsa-mir-200b | hsa-mir-212 | hsa-mir-184 | hsa-mir-99b | hsa-mir-187 | hsa-mir-205 |
| hsa-mir-200c | hsa-mir-29a | hsa-mir-187 | | hsa-mir-18b | hsa-mir-20b |
| hsa-mir-202 | hsa-mir-29c | hsa-mir-18b | | hsa-mir-192 | hsa-mir-21 |
| hsa-mir-203 | hsa-mir-30b | hsa-mir-192 | | hsa-mir-193a | hsa-mir-210 |
| hsa-mir-205 | hsa-mir-30d | hsa-mir-193a | | hsa-mir-193b | hsa-mir-216a |
| hsa-mir-206 | hsa-mir-331 | hsa-mir-193b | | hsa-mir-195 | hsa-mir-221 |
| hsa-mir-20b | hsa-mir-345 | hsa-mir-195 | | hsa-mir-19b-2 | hsa-mir-222 |
| hsa-mir-21 | hsa-mir-34a | hsa-mir-19b-2 | | hsa-mir-200c | hsa-mir-23a |
| hsa-mir-210 | hsa-mir-377 | hsa-mir-200c | | hsa-mir-202 | hsa-mir-27a |
| hsa-mir-212 | hsa-mir-409 | hsa-mir-202 | | hsa-mir-203 | hsa-mir-298 |
| hsa-mir-217 | hsa-mir-495 | hsa-mir-203 | | hsa-mir-205 | hsa-mir-301b |
| hsa-mir-221 | hsa-mir-520h | hsa-mir-205 | | hsa-mir-206 | hsa-mir-30a |
| hsa-mir-222 | hsa-mir-542 | hsa-mir-206 | | hsa-mir-20b | hsa-mir-331 |
| hsa-mir-23a | hsa-mir-572 | hsa-mir-20b | | hsa-mir-21 | hsa-mir-345 |
| hsa-mir-27a | hsa-mir-648 | hsa-mir-21 | | hsa-mir-210 | hsa-mir-34a |
| hsa-mir-296 | hsa-mir-96 | hsa-mir-210 | | hsa-mir-212 | hsa-mir-373 |
| hsa-mir-29a | hsa-mir-99b | hsa-mir-212 | | hsa-mir-217 | hsa-mir-374a |
| hsa-mir-29c | | hsa-mir-217 | | hsa-mir-221 | hsa-mir-377 |
| hsa-mir-30a | | hsa-mir-221 | | hsa-mir-222 | hsa-mir-485 |
| hsa-mir-30b | | hsa-mir-222 | | hsa-mir-23a | hsa-mir-493 |
| hsa-mir-30d | | hsa-mir-23a | | hsa-mir-24-2 | hsa-mir-495 |
| hsa-mir-320 | | hsa-mir-24-2 | | hsa-mir-27a | hsa-mir-496 |
| hsa-mir-331 | | hsa-mir-27a | | hsa-mir-29a | hsa-mir-523 |
| hsa-mir-345 | | hsa-mir-29a | | hsa-mir-29b-1 | hsa-mir-525 |
| hsa-mir-34a | | hsa-mir-29b-1 | | hsa-mir-29b-2 | hsa-mir-542 |
| hsa-mir-377 | | hsa-mir-29b-2 | | hsa-mir-29c | hsa-mir-568 |
| hsa-mir-379 | | hsa-mir-29c | | hsa-mir-30a | hsa-mir-570 |
| hsa-mir-411 | | hsa-mir-30a | | hsa-mir-30b | hsa-mir-572 |
| hsa-mir-429 | | hsa-mir-30b | | hsa-mir-30c-2 | hsa-mir-633 |
| hsa-mir-494 | | hsa-mir-30c-2 | | hsa-mir-30d | hsa-mir-648 |
| hsa-mir-497 | | hsa-mir-30d | | hsa-mir-3178 | hsa-mir-92b |
| hsa-mir-648 | | hsa-mir-3178 | | hsa-mir-331 | hsa-mir-99b |
| hsa-mir-96 | | hsa-mir-331 | | hsa-mir-345 | hsa-mir-451 |
| hsa-mir-99b | | hsa-mir-345 | | hsa-mir-34a | |
| | | hsa-mir-34a | | hsa-mir-363 | |
| | | hsa-mir-363 | | hsa-mir-369 | |
| | | hsa-mir-369 | | hsa-mir-371 | |
| | | hsa-mir-371 | | hsa-mir-372 | |
| | | hsa-mir-372 | | hsa-mir-373 | |
| | | hsa-mir-373 | | hsa-mir-374a | |
| | | hsa-mir-374a | | hsa-mir-379 | |
| | | hsa-mir-379 | | hsa-mir-409 | |
| | | hsa-mir-409 | | hsa-mir-422a | |
| | | hsa-mir-422a | | hsa-mir-494 | |
| | | hsa-mir-494 | | hsa-mir-497 | |
| | | hsa-mir-495 | | hsa-mir-505 | |
| | | hsa-mir-497 | | hsa-mir-518c | |
| | | hsa-mir-505 | | hsa-mir-519e | |
| | | hsa-mir-518c | | hsa-mir-520a | |
| | | hsa-mir-519e | | hsa-mir-520g | |
| | | hsa-mir-520a | | hsa-mir-522 | |
| | | hsa-mir-520g | | hsa-mir-523 | |
| | | hsa-mir-522 | | hsa-mir-572 | |
| | | hsa-mir-523 | | hsa-mir-648 | |
| | | hsa-mir-572 | | hsa-mir-654 | |
| | | hsa-mir-648 | | hsa-mir-665 | |
| | | hsa-mir-654 | | hsa-mir-769 | |
| | | hsa-mir-665 | | hsa-mir-92b | |
| | | hsa-mir-769 | | hsa-mir-96 | |
| | | hsa-mir-92b | | hsa-mir-99b | |
| | | hsa-mir-96 | | hsa-mir-103-1 | |
| | | hsa-mir-99b | | hsa-mir-103-2 | |
| | | | | hsa-mir-107 | |

TABLE 4
MiRs involved in neonatal and hereditary diseases, respiratory diseases,
urogenital diseases in men, urogenital diseases in women, disorders
of the immune system and musculoskeletal disorders

| Neonatal and | | Urogenital | Urogenital | Disorders | |
|---|---|---|---|---|---|
| hereditary | Respiratory | diseases | diseases | of the | Musculoskeletal |
| diseases | diseases | in men | in women | immune system | disorders |
| hsa-let-7a-2 | hsa-let-7a-2 | hsa-let-7a-2 | hsa-let-7a-2 | hsa-let-7a-2 | hsa-let-7b |
| hsa-let-7a-3 | hsa-let-7a-3 | hsa-let-7a-3 | hsa-let-7a-3 | hsa-let-7a-3 | hsa-let-7g |
| hsa-let-7b | hsa-let-7b | hsa-let-7b | hsa-let-7b | hsa-let-7b | hsa-mir-106a |
| hsa-let-7d | hsa-let-7d | hsa-let-7d | hsa-let-7d | hsa-let-7d | hsa-mir-10a |
| hsa-let-7e | hsa-let-7e | hsa-let-7e | hsa-let-7e | hsa-let-7e | hsa-mir-10b |
| hsa-let-7g | hsa-let-7g | hsa-let-7g | hsa-let-7g | hsa-let-7g | hsa-mir-1246 |
| hsa-let-7i | hsa-let-7i | hsa-let-7i | hsa-let-7i | hsa-let-7i | hsa-mir-132 |
| hsa-mir-100 | hsa-mir-106a | hsa-mir-106a | hsa-mir-106a | hsa-mir-100 | hsa-mir-134 |
| hsa-mir-106a | hsa-mir-10a | hsa-mir-10b | hsa-mir-10b | hsa-mir-106a | hsa-mir-142 |
| hsa-mir-10a | hsa-mir-10b | hsa-mir-132 | hsa-mir-132 | hsa-mir-10a | hsa-mir-145 |
| hsa-mir-10b | hsa-mir-1197 | hsa-mir-142 | hsa-mir-142 | hsa-mir-10b | hsa-mir-146a |
| hsa-mir-122 | hsa-mir-122 | hsa-mir-143 | hsa-mir-143 | hsa-mir-122 | hsa-mir-150 |
| hsa-mir-1246 | hsa-mir-1246 | hsa-mir-145 | hsa-mir-145 | hsa-mir-1246 | hsa-mir-155 |
| hsa-mir-125a | hsa-mir-125a | hsa-mir-150 | hsa-mir-150 | hsa-mir-125a | hsa-mir-181c |
| hsa-mir-130b | hsa-mir-130b | hsa-mir-181c | hsa-mir-181c | hsa-mir-130b | hsa-mir-182 |
| hsa-mir-132 | hsa-mir-132 | hsa-mir-182 | hsa-mir-182 | hsa-mir-132 | hsa-mir-183 |
| hsa-mir-134 | hsa-mir-134 | hsa-mir-183 | hsa-mir-183 | hsa-mir-134 | hsa-mir-184 |
| hsa-mir-141 | hsa-mir-135b | hsa-mir-184 | hsa-mir-184 | hsa-mir-141 | hsa-mir-18b |
| hsa-mir-142 | hsa-mir-141 | hsa-mir-192 | hsa-mir-192 | hsa-mir-142 | hsa-mir-192 |
| hsa-mir-143 | hsa-mir-142 | hsa-mir-195 | hsa-mir-195 | hsa-mir-143 | hsa-mir-195 |
| hsa-mir-145 | hsa-mir-143 | hsa-mir-200a | hsa-mir-200a | hsa-mir-144 | hsa-mir-200a |
| hsa-mir-146a | hsa-mir-144 | hsa-mir-202 | hsa-mir-202 | hsa-mir-145 | hsa-mir-202 |
| hsa-mir-146b | hsa-mir-145 | hsa-mir-203 | hsa-mir-203 | hsa-mir-146a | hsa-mir-203 |
| hsa-mir-148a | hsa-mir-146a | hsa-mir-205 | hsa-mir-205 | hsa-mir-146b | hsa-mir-205 |
| hsa-mir-154 | hsa-mir-146b | hsa-mir-21 | hsa-mir-21 | hsa-mir-148a | hsa-mir-206 |
| hsa-mir-155 | hsa-mir-148a | hsa-mir-210 | hsa-mir-210 | hsa-mir-150 | hsa-mir-20b |
| hsa-mir-181a-2 | hsa-mir-150 | hsa-mir-212 | hsa-mir-212 | hsa-mir-154 | hsa-mir-21 |
| hsa-mir-181c | hsa-mir-154 | hsa-mir-29a | hsa-mir-29a | hsa-mir-155 | hsa-mir-210 |
| hsa-mir-181d | hsa-mir-155 | hsa-mir-29c | hsa-mir-29c | hsa-mir-181a-2 | hsa-mir-27a |
| hsa-mir-182 | hsa-mir-181a-2 | hsa-mir-30b | hsa-mir-30b | hsa-mir-181c | hsa-mir-29a |
| hsa-mir-183 | hsa-mir-181c | hsa-mir-30d | hsa-mir-30d | hsa-mir-181d | hsa-mir-29c |
| hsa-mir-18b | hsa-mir-182 | hsa-mir-331 | hsa-mir-331 | hsa-mir-182 | hsa-mir-30b |
| hsa-mir-192 | hsa-mir-183 | hsa-mir-34a | hsa-mir-34a | hsa-mir-183 | hsa-mir-345 |
| hsa-mir-193a | hsa-mir-18b | hsa-mir-377 | hsa-mir-377 | hsa-mir-184 | hsa-mir-377 |
| hsa-mir-193b | hsa-mir-192 | hsa-mir-409 | hsa-mir-409 | hsa-mir-187 | hsa-mir-409 |
| hsa-mir-195 | hsa-mir-193a | hsa-mir-495 | hsa-mir-495 | hsa-mir-18b | hsa-mir-495 |
| hsa-mir-202 | hsa-mir-193b | hsa-mir-520h | hsa-mir-520h | hsa-mir-192 | hsa-mir-520h |
| hsa-mir-203 | hsa-mir-195 | hsa-mir-542 | hsa-mir-542 | hsa-mir-193a | hsa-mir-542 |
| hsa-mir-206 | hsa-mir-200a | hsa-mir-572 | hsa-mir-572 | hsa-mir-193b | hsa-mir-572 |
| hsa-mir-20b | hsa-mir-200b | hsa-mir-648 | hsa-mir-648 | hsa-mir-195 | hsa-mir-648 |
| hsa-mir-21 | hsa-mir-200c | hsa-mir-96 | hsa-mir-96 | hsa-mir-200a | hsa-mir-96 |
| hsa-mir-210 | hsa-mir-202 | hsa-mir-99b | hsa-mir-99b | hsa-mir-200b | hsa-mir-99b |
| hsa-mir-212 | hsa-mir-203 | | | hsa-mir-202 | |
| hsa-mir-217 | hsa-mir-205 | | | hsa-mir-203 | |
| hsa-mir-221 | hsa-mir-206 | | | hsa-mir-205 | |
| hsa-mir-222 | hsa-mir-20b | | | hsa-mir-20b | |
| hsa-mir-23a | hsa-mir-21 | | | hsa-mir-21 | |
| hsa-mir-27a | hsa-mir-210 | | | hsa-mir-210 | |
| hsa-mir-29a | hsa-mir-212 | | | hsa-mir-212 | |
| hsa-mir-29c | hsa-mir-221 | | | hsa-mir-216a | |
| hsa-mir-30a | hsa-mir-222 | | | hsa-mir-217 | |
| hsa-mir-30b | hsa-mir-23a | | | hsa-mir-221 | |
| hsa-mir-30c-2 | hsa-mir-24-2 | | | hsa-mir-222 | |
| hsa-mir-30d | hsa-mir-27a | | | hsa-mir-23a | |
| hsa-mir-3178 | hsa-mir-298 | | | hsa-mir-27a | |
| hsa-mir-331 | hsa-mir-29a | | | hsa-mir-298 | |
| hsa-mir-345 | hsa-mir-29c | | | hsa-mir-29a | |
| hsa-mir-34a | hsa-mir-30a | | | hsa-mir-29c | |
| hsa-mir-363 | hsa-mir-30b | | | hsa-mir-301b | |
| hsa-mir-369 | hsa-mir-30d | | | hsa-mir-30a | |
| hsa-mir-371 | hsa-mir-320 | | | hsa-mir-30b | |
| hsa-mir-372 | hsa-mir-331 | | | hsa-mir-30c-2 | |
| hsa-mir-373 | hsa-mir-337 | | | hsa-mir-30d | |
| hsa-mir-374a | hsa-mir-345 | | | hsa-mir-3178 | |
| hsa-mir-377 | hsa-mir-34a | | | hsa-mir-331 | |
| hsa-mir-379 | hsa-mir-369 | | | hsa-mir-345 | |
| hsa-mir-409 | hsa-mir-374a | | | hsa-mir-34a | |
| hsa-mir-422a | hsa-mir-374b | | | hsa-mir-363 | |
| hsa-mir-494 | hsa-mir-377 | | | hsa-mir-369 | |
| hsa-mir-495 | hsa-mir-379 | | | hsa-mir-371 | |
| hsa-mir-497 | hsa-mir-382 | | | hsa-mir-372 | |
| hsa-mir-505 | hsa-mir-409 | | | hsa-mir-373 | |
| hsa-mir-518c | hsa-mir-411 | | | hsa-mir-374a | |
| hsa-mir-519e | hsa-mir-422a | | | hsa-mir-377 | |
| hsa-mir-520a | hsa-mir-429 | | | hsa-mir-379 | |
| hsa-mir-520g | hsa-mir-450b | | | hsa-mir-409 | |
| hsa-mir-522 | hsa-mir-487a | | | hsa-mir-422a | |
| hsa-mir-523 | hsa-mir-493 | | | hsa-mir-485 | |
| hsa-mir-542 | hsa-mir-494 | | | hsa-mir-493 | |
| hsa-mir-572 | hsa-mir-495 | | | hsa-mir-494 | |
| hsa-mir-648 | hsa-mir-505 | | | hsa-mir-495 | |
| hsa-mir-654 | hsa-mir-518d | | | hsa-mir-496 | |
| hsa-mir-665 | hsa-mir-518e | | | hsa-mir-497 | |
| hsa-mir-769 | hsa-mir-518f | | | hsa-mir-505 | |
| hsa-mir-92b | hsa-mir-520a | | | hsa-mir-518c | |
| hsa-mir-96 | hsa-mir-520d | | | hsa-mir-519e | |
| hsa-mir-99b | hsa-mir-520e | | | hsa-mir-520a | |
| | hsa-mir-523 | | | hsa-mir-520g | |
| | hsa-mir-542 | | | hsa-mir-520h | |
| | hsa-mir-543 | | | hsa-mir-522 | |
| | hsa-mir-570 | | | hsa-mir-523 | |
| | hsa-mir-572 | | | hsa-mir-525 | |
| | hsa-mir-648 | | | hsa-mir-542 | |
| | hsa-mir-656 | | | hsa-mir-568 | |
| | hsa-mir-668 | | | hsa-mir-570 | |
| | hsa-mir-758 | | | hsa-mir-572 | |
| | hsa-mir-769 | | | hsa-mir-633 | |
| | hsa-mir-96 | | | hsa-mir-648 | |
| | hsa-mir-99b | | | hsa-mir-654 | |
| | | | | hsa-mir-665 | |
| | | | | hsa-mir-769 | |
| | | | | hsa-mir-92b | |
| | | | | hsa-mir-96 | |
| | | | | hsa-mir-99b | |

TABLE 5
The top 10 miRs involved in cancers, bacterial and fungal infections, immune system disorders, cardiovascular diseases, hereditary
congenital diseases, skin diseases, musculoskeletal disorders, eye diseases and diseases of the digestive system.

| | | | | | | | | Diseases |
|---|---|---|---|---|---|---|---|---|
| | bacterial | Immune | | Hereditary | | | | of the |
| | infections - | system | Cardiovascular | congenital | Skin | Musculoskeletal | Eye | digestive |
| Cancer | Mycosis | disorders | diseases | diseases | diseases | disorders | diseases | system |
| hsa-mir-21 | hsa-mir-155 | hsa-mir-192 | hsa-mir-192 | hsa-mir-192 | hsa-mir-182 | hsa-mir-192 | hsa-mir-192 | hsa-mir-122 |
| hsa-mir-145 | hsa-mir-21 | hsa-mir-221 | hsa-mir-21 | hsa-mir-221 | hsa-mir-221 | hsa-mir-210 | hsa-mir-145 | hsa-mir-192 |
| hsa-mir-192 | hsa-mir-203 | hsa-mir-141 | hsa-mir-122 | hsa-let-7i | hsa-mir-203 | hsa-mir-182 | hsa-let-7g | hsa-mir-21 |
| hsa-mir-205 | hsa-mir-195 | hsa-mir-145 | hsa-mir-145 | hsa-mir-122 | hsa-mir-106a | hsa-mir-183 | hsa-mir-331 | hsa-mir-210 |
| hsa-mir-143 | hsa-let-7b | hsa-mir-182 | hsa-mir-210 | hsa-mir-141 | hsa-mir-155 | hsa-mir-96 | hsa-mir-99b | hsa-mir-99b |
| hsa-mir-10a | hsa-let-7d | hsa-mir-21 | hsa-mir-29c | hsa-mir-182 | hsa-mir-29a | hsa-mir-106a | hsa-mir-182 | hsa-let-7b |
| hsa-mir-106a | hsa-mir-145 | hsa-mir-203 | hsa-mir-497 | hsa-mir-155 | hsa-mir-210 | hsa-mir-10b | hsa-mir-183 | hsa-let-7d |
| hsa-mir-146a | hsa-mir-1246 | hsa-let-7i | hsa-mir-96 | hsa-mir-145 | hsa-mir-148a | hsa-mir-1246 | hsa-mir-195 | hsa-mir-10a |
| hsa-mir-182 | hsa-mir-192 | hsa-mir-106a | hsa-mir-132 | hsa-mir-222 | hsa-mir-141 | hsa-mir-132 | hsa-mir-106a | hsa-let-7g |
| hsa-mir-221 | hsa-let-7g | hsa-mir-122 | hsa-mir-141 | hsa-mir-20b | hsa-let-7i | hsa-mir-134 | hsa-mir-10a | hsa-let-7i |

TABLE 6
The top 10 miRs involved in diseases of the endocrine system, diseases of the nervous system, diseases related
to viruses, diseases related to nutrition and metabolism, lymphatic diseases and hemopathies, neonatal and
hereditary diseases, respiratory diseases , urogenital diseases in men, urogenital diseases in women.

| | | | Diseases | | | | | |
|---|---|---|---|---|---|---|---|---|
| diseases | diseases | | related to | Lymphatic | Neonatal | | | |
| of the | of the | Diseases | nutrition | diseases | and | | Urogenital | Urogenital |
| endocrine | nervous | related to | and | and | hereditary | Respiratory | diseases | diseases |
| system | system | viruses | metabolism | hemopathies | diseases | diseases | in men | in women |
| hsa-mir-145 | hsa-mir-192 | hsa-mir-122 | hsa-mir-192 | hsa-mir-106a | hsa-mir-192 | hsa-mir-21 | hsa-mir-21 | hsa-mir-21 |
| hsa-mir-21 | hsa-mir-122 | hsa-mir-192 | hsa-mir-122 | hsa-mir-205 | hsa-mir-221 | hsa-mir-145 | hsa-let-7b | hsa-let-7b |
| hsa-mir-192 | hsa-mir-145 | hsa-mir-195 | hsa-mir-21 | hsa-mir-145 | hsa-mir-182 | hsa-mir-143 | hsa-mir-145 | hsa-mir-145 |
| hsa-mir-181c | hsa-mir-141 | hsa-mir-18b | hsa-mir-141 | hsa-mir-125a | hsa-let-7i | hsa-mir-144 | hsa-mir-106a | hsa-mir-181c |
| hsa-mir-182 | hsa-mir-142 | hsa-mir-21 | hsa-mir-145 | hsa-mir-141 | hsa-mir-122 | hsa-mir-182 | hsa-let-7d | hsa-let-7d |
| hsa-mir-183 | hsa-mir-143 | hsa-mir-1246 | hsa-mir-142 | hsa-mir-143 | hsa-mir-141 | hsa-mir-195 | hsa-mir-181c | hsa-mir-106a |
| hsa-mir-96 | hsa-mir-146a | hsa-mir-212 | hsa-mir-143 | hsa-let-7d | hsa-mir-145 | hsa-mir-192 | hsa-mir-192 | hsa-mir-192 |
| hsa-mir-210 | hsa-mir-100 | hsa-mir-34a | hsa-mir-155 | hsa-let-7i | hsa-mir-155 | hsa-mir-221 | hsa-mir-195 | hsa-mir-195 |
| hsa-mir-212 | hsa-mir-155 | hsa-mir-106a | hsa-mir-182 | hsa-mir-331 | hsa-mir-222 | hsa-mir-222 | hsa-mir-200a | hsa-mir-200a |
| hsa-mir-132 | hsa-mir-182 | hsa-mir-10a | hsa-mir-183 | hsa-mir-192 | hsa-mir-20b | hsa-mir-27a | hsa-mir-202 | hsa-mir-202 |

The following examples will better illustrate the invention without limiting its scope.

### EXAMPLES

### Example 1

### miPEP 125a-1 and miPEP 145-2

A transcriptomic analysis carried out by the inventors has revealed that the miR 125a-1 and miR 145-2 are overexpressed during the differentiation of the cells into osteoblasts (unpublished results).

The miPEPs 125a-1 and 145-2 sequences were identified from the primary transcripts of miRs 125a-1 and 145-2.

The respective effects of miPEP 125a-1 and miPEP 145-2 on differentiation of mesenchymal stem cells were analyzed by measuring the expression of the iBSP, PTHR1, STMN2 and OSTERIX genes that correspond to markers of osteogenesis and of osteoblast differentiation (Chiellini et al., Biochem Biophys Res Commun, 374(1):64-8, 2008; Cordonnier et al., Tissue Eng, 17(3): 249-259, 2011).

The experiments were carried out with two fragments of 10 amino acids respectively corresponding to the N-terminal part of the miPEP 125a-1 (=miPEP//125a-1 fragment) and to the N-terminal part of the miPEP 145-2 (=fragment miPEP//145-2). Mesenschymal stem cells from 4 different donors were cultured for 24 hours in PROLIF medium (αMEM+10% FCS) with or without miPEP// then for 3 days in DIF+BMP4 medium (αMEM+2% FCS +b-Glycerophosphate+Ascorbic acid+BMP4), DIF ΔBMP4 medium (αMEM+2% FCS +b-Glycerophosphate+Ascorbic acid) or PROLIF medium (αMEM+10% FCS), with or without miPEP//.

The expression of the iBSP, PTHR1, STMN2 and OSTERIX genes was then measured in mesenchymal stem cells cultured in the presence or absence of a miPEP*.

### 1.1 - Results

### In DIF +BMP4 Medium

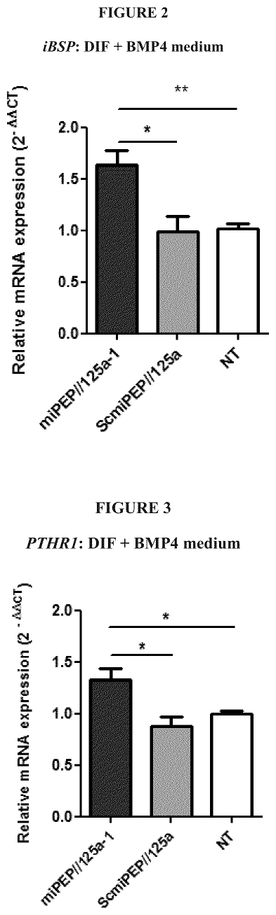

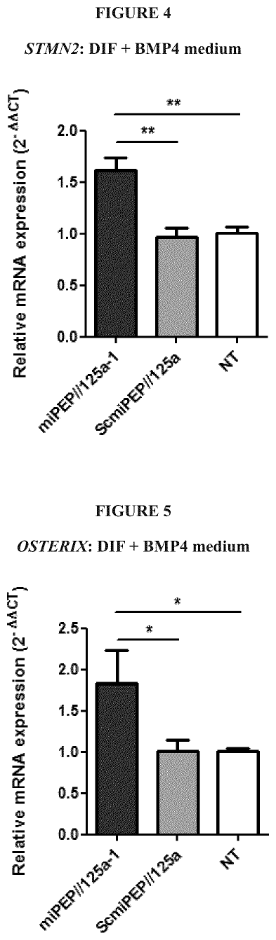

The miPEP//125a-1 increases the expression of the iBSP, PTHR1, STMN2 and OSTERIX genes (FIGS. 2, 3, 4 and 5).

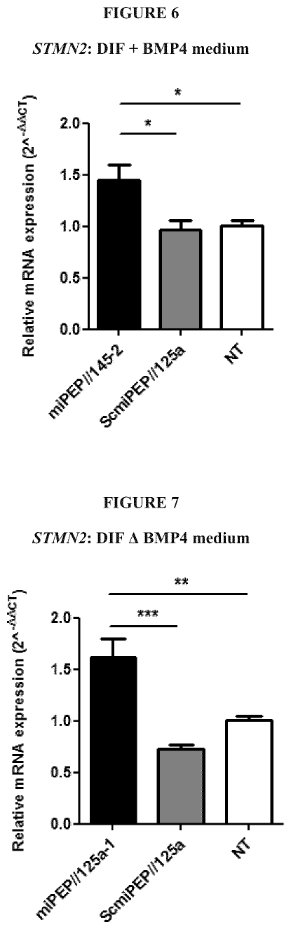

The miPEP//145-2 increases the expression of the STMN2 gene (FIG. 6).

### In DIF γBMP4 Medium

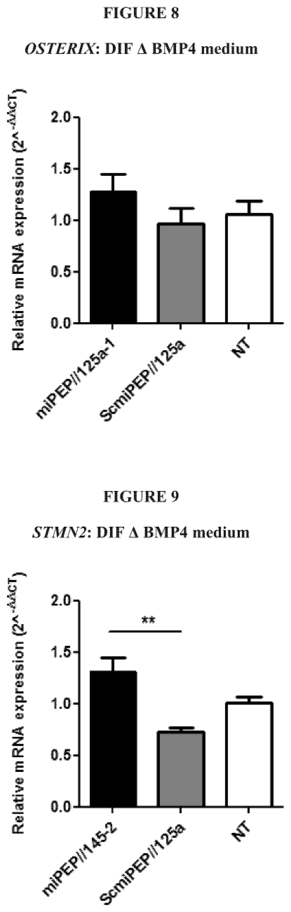

The miPEP//125a-1 increases the expression of the STMN2 and OSTERIX genes (FIGS. 7 and 8).

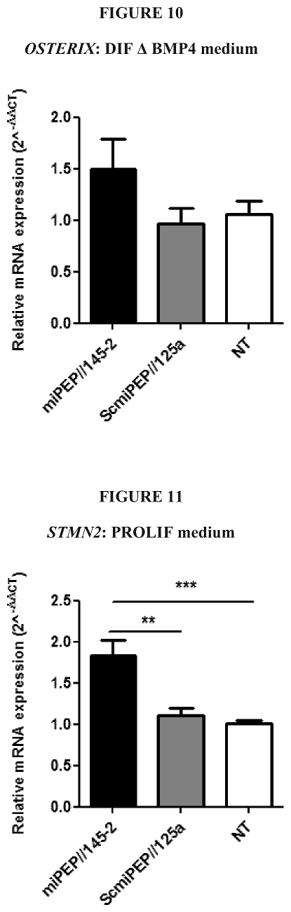

The miPEP//145-2 increases the expression of the STMN2 and OSTERIX genes (FIGS. 9 and 10).

### In PROLIF Medium

The miPEP//145-2 increases the expression STMN2 gene (FIG. 11).

The above results show that miPEP//125a-1 and miPEP//145-2 increase the expression of osteoblast marker genes, indicating that these miPEPs promote osteoblast differentiation of mesenchymal stem cells.

### 1.2—Materials

MEMα GIBCO (reference 22561-021)=>“αMEM”,
Fetal bovine serum (FBS),
Penicillin/streptomycin GIBCO (reference 15140-122)=>“P/S”,
Dulbecco's PBS GIBCO (reference 14190-169)=>“PBS”,
12-well plate,
βglycerophosphate SIGMA (reference G9422),
Ascorbic acid SIGMA (reference A8960),
BMP4 PEPROTECH (reference 120-05ET)=>“BMP4”,
miPEP//125a-1 (MSLCLSPSLT, SEQ ID NO: 11 752),
miPEP//145-2 (MVGLNPPLWQ, SEQ ID NO: 11 751),
“scramble” control peptide ScmiPEP//125a (IQVGHEDETD, SEQ ID NO: 11 758).

### 1.3—Methods

a. Composition of Culture Media
Proliferation medium (PROLIF): αMEM+10% FBS+1% P/S
Differentiation medium+BMP4 (DIF+BMP4): αMEM+2% FBS+1% P/S+βglycerophosphate (10 mM)+Ascorbic acid (50 μM)+BMP4 (50 ng/mL)
Differentiation medium without BMP4 (DIF ΔBMP4): αMEM+2% FBS+1% P/S+βglycerophosphate (10 mM)+Ascorbic acid (50 μM)
   b. Resuspension of Micropeptides

The miPEP//145-2 was synthesized by Smartbioscience and dissolved at 2 mM in water. The miPEP//125a-1 was synthesized by Smartbioscience and dissolved in water+0.1% ammonium.

c. Seeding of the Cells

The mesenchymal stem cells were seeded in 12-well plates at 70,000 cells/well in 1 mL of PROLIF medium. The cells were then incubated for 72 h at 37° C. in a humid atmosphere with 5% CO₂.

d. Cell Treatment with miPEPs

After 72 hours of incubation, the cells were pre-treated with 100 μM of miPEP or “scramble” peptide diluted in PROLIF medium, and then incubated at 37° C. for 24 hours. In parallel, cells treated with PROLIF±water or water+0.1% ammonium medium were used as a negative control.

For 3 days, the cells were treated every 24 h with 100 μM of diluted miPEP either in DIF+BMP4 medium, or in DIF ΔBMP4 medium, or in PROLIF medium. Cells untreated or treated with water or water+0.1% ammonium were used as controls. The cells are incubated at 37° C. After 3 days of treatment, the culture supernatants were removed, the cell mats were washed once with PBS, and then the plates were frozen at −80° C. until the total RNAs were extracted.

e. Extraction of Total RNA

Total RNAs were extracted according to the supplier's recommendations with the miRNeasy microkit kit (Qiagen).

f. Reverse Transcription Reaction

The RNAs were reverse transcribed with the High Capacity cDNA reverse transcription kit (Applied Biosystem). 600 ng of RNA were added to 2 μL of RT buffer (10×), 2 μL of Random primer (10×), 0.8 μL of dNTPs (25×), 0.5 μL of RNAse OUT (40 U/μL) and 1 μL of multiscribe reverse transcriptase (50 U/μL) in a total volume of 20 μL. The reverse transcription reaction was carried out at 25° C. for 10 min and then 42° C. for 2 h. The activity of the enzyme was stopped by incubating the mixture at 85° C. for 5 min.

g. Evaluation of Gene Expression by Quantitative RT-PCR

The expression of osteoblastic transcription factors iBSP, PTHR1, STMN2 and OSTERIX was evaluated by quantitative RT-PCR with the SsoFast EvagGreen kit. For the RT-PCR reaction, 3 μL of reverse transcriptase diluted ⅛ were added to 1× of SsoFast EvaGreen, 10 μM of each primer and RNase free water in a final volume of 10 μL. The reaction was subjected to the following temperatures: 3 min pre-amplification at 95° C., 40 cycles of 10 sec at 95° C. and 30 sec at 60° C. in a thermal cycler (CFX96 Real-time PCR detection system, Biorad). PPIA was used as a reference gene to normalize quantitative RT-PCR.

For all PCR reactions, two replicates were used for each biological sample.

TABLE 7
Sequences of the primers.

| | | Forward primer (5′-3′) | Reverse primer (5′-3′) |
|---|---|---|---|
| Reference | PPIA | TCT TTG GGA CCT TGT CTG | GCC GAG GAA AAC CGT |
| gene | | CAA (SEQ ID NO: 11 759) | GTA CTA T |
| | | | (SEQ ID NO: 11 760) |
| Osteoblastic | iBSP | GGG CAG TAG TGA CTC ATC | CTC CAT AGC CCA GTG |
| marker genes | | CGA AG (SEQ ID NO: 11 761) | TTG TAG CAG |
| | | | (SEQ ID NO: 11 762) |
| | OSX | CTC CTG CGA CTG CCC TAA | GCC TTG CCA TAC ACC |
| | | T (SEQ ID NO: 11 763) | TTG C (SEQ ID NO: 11 764) |
| | PTHR1 | ACA TCT GCG TCC ACA TCA | CCG TTC ACG AGT CTC |
| | | GG (SEQ ID NO: 11 765) | ATT GGT G |
| | | | (SEQ ID NO: 11 766) |
| | STMN2 | GCG GAG GAA AAg CTG | TCC GCA GCA TGC CTC |
| | | ATC CTG A | TCC TT (SEQ ID NO: 11 768) |
| | | (SEQ ID NO: 11 767) | |

### Example 2

### Cell Penetration of miPEPs

The entry of miPEPs into mesenchymal stem cells (MSCs) was analyzed by fluorescence microscopy using fluorescein (FAM)-labeled miPEP//15a-16-1, fused or not to a peptide promoting cell penetration (TAT peptide).

### 2.1—Results

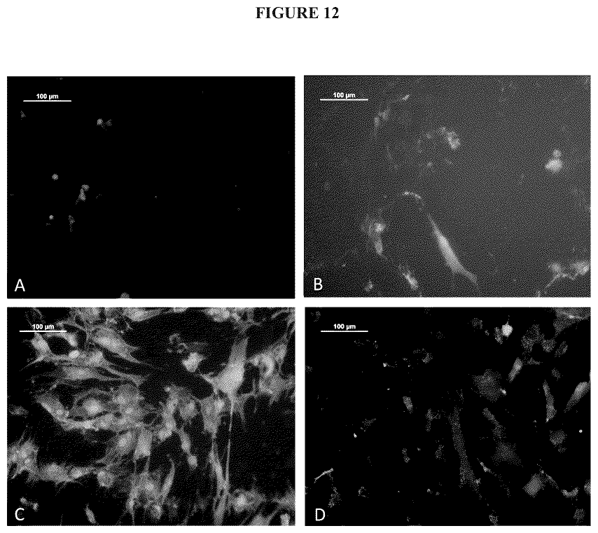

In a first experiment, undifferentiated MSCs were incubated for 7 h with 5 μM, 10 μM, 50 μM or 100 μM of miPEP//15a-16-1-FAM (FIG. 12).

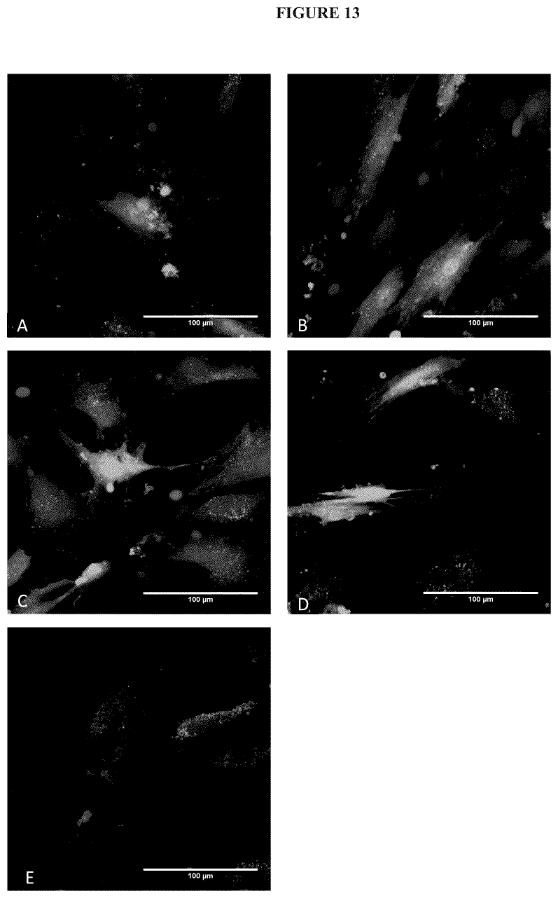

In a second experiment, undifferentiated MSCs were incubated with 100 μM of miPEP//15a-16-1-FAM for 2 h, 4 h, 6 h, 8 h or 24 h (FIG. 13).

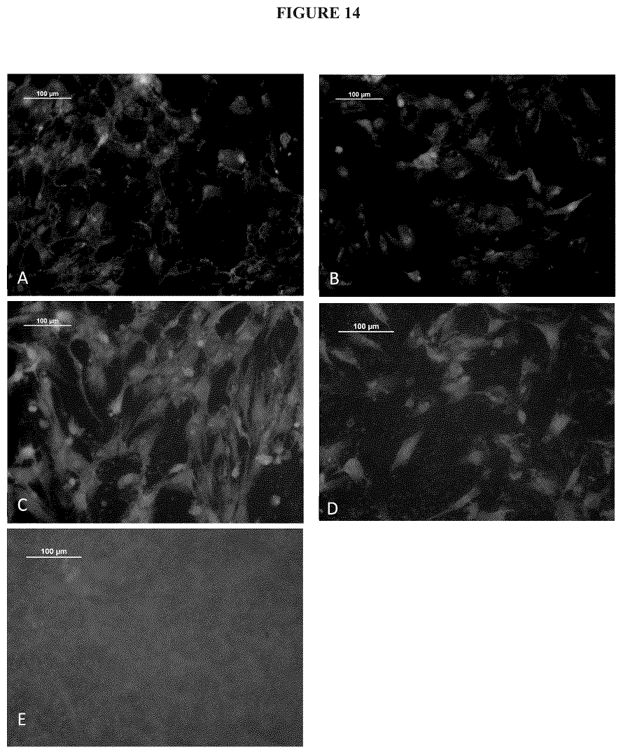

In a third experiment, undifferentiated MSCs were incubated for 7 h with 5 μM, 10 μM, 20 μM, 50 μM or 100 μM of miPEP//15a-16-1-FAM-TAT (FIGS. 14).

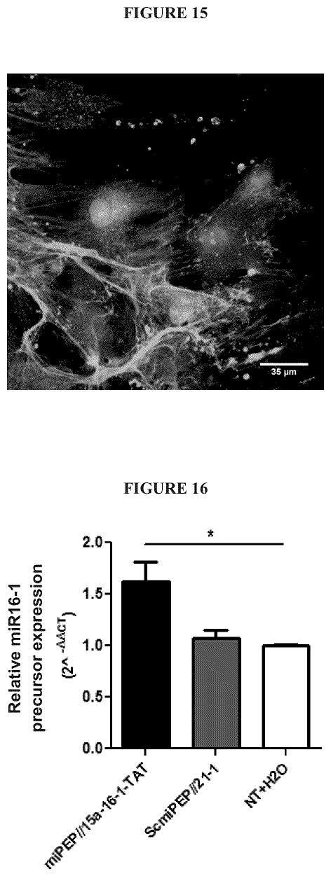

In a fourth experiment, undifferentiated MSCs were incubated for 2 h, with 10 μM of miPEP//15 a-16-1-FAM- TAT (FIG. 15).

The above results indicate, on the one hand, that the miPEP//15a-16-1-FAM homogeneously penetrates the MSCs when the MSCs are treated with at least 50 μM of miPEP, and on the other hand, that the miPEP is captured in the core of the MSCs.

The strongest fluorescence is observed from 4 h to 8 h after the treatment with the miPEP.

These results also indicate that the addition of the TAT peptide to the miPEP promotes cell penetration by a factor of 10 relative to the cellular penetration of the miPEP without the TAT peptide.

### 2.2—Materials

MEMα GIBCO (reference 22561-021)=>“αMEM”,
Fetal bovine serum (FBS),
Penicillin/streptomycin GIBCO (reference 15140-122)=>“P/S”,
Dulbecco's PBS GIBCO (reference 14190-169)=>“PBS”,
8-well IBIDI blade (15μ slide 8 well IBIDi; Biovalley),
DRAQS (5 mM) Biostatus,
Formaldehyde solution 37%,

miPEP//15a-16-1
(MFKHRFFYMH, SEQ ID NO: 11 753),
miPEP//15a-16-1-TAT
(MFKHRFFYMH-YGRKKRRQRRR, SEQ ID NO: 11 757).

### 2.3—Methods

Undifferentiated MSCs are incubated in αMEM+10% FBS+1% P/S medium in the presence of fluorescein (FAM) labeled miPEP. After incubation from 2 h to 10 h, the wells are washed 3 times with PBS and then the cells are fixed for 5 min with 3.8% formaldehyde. The nuclei are marked with the Draq5 diluted 1/1 000. The cells are then observed under a confocal microscope in fluorescence.

### Example 3

### miPEP 15a-16-1

A transcriptomic analysis carried out by the inventors has revealed that the miR 15a and miR 16-1 are overexpressed during the differentiation of the cells into osteoblasts (unpublished results).

The miPEP 15a-16-1 sequence was identified from the primary transcript of 15a-16-1.

The effect of miPEP 15a-16-1 on miR16-1 accumulation was analyzed in mesenchymal stem cells (MSCs). The experiments were carried out with a fragment of 10 amino acids respectively corresponding to the N-terminal portion of the miPEP 15a-16-1 (=fragment miPEP//15a-16-1).

MSCs from three different patients were treated with 10 μM of miPEP//15a-16-1 fused to TAT peptide (miPEP//15a-16-1-TAT) for 3h, then the amount of miR16-1 was measured by RT-qPCR.

### 3.1—Results

Treatment with miPEP//15a-16-1-TAT results in an increase in the amount of miR 16-1 (FIG. 16).

### 3.2—Materials

MEMα GIBCO (reference 22561-021)=>“αMEM”,
Fetal bovine serum (FBS),
Penicillin/streptomycin GIBCO (reference 15140-122)=>“P/S”,
Dulbecco's PBS GIBCO (reference 14190-169) “PBS”,

miPEP//15a-16-1-TAT
(MFKHRFFYMH-YGRKKRRQRRR, SEQ ID NO: 11 757),
ScmiPEP//21 (RPHLRMELPV).

### 3.3—Methods

a. Resuspension of Micropeptides

Peptides were synthesized by Smartbioscience and dissolved at 2 mM in water.

b. Seeding of the Cells

The MSCs were seeded in wells of 12-well plates at 72,900 cells per well under 1 mL of PROLIF medium. The cells were then incubated for 72 h at 37° C. in a humid atmosphere with 5% CO₂.

c. Cell Treatment with miPEPs

After 24 h of incubation, the cells were treated with 10 μM of miPEP//15a-16-1-TAT or 100 μM of ScmiPEP21 diluted in PROLIF medium and then incubated at 37° C. for 1 h, 2 h, 3 h, 4 h and 6 h. In parallel, cells treated with PROLIF±water medium were used as a negative control.

After treatment from 1 h to 6 h, the culture supernatants were removed and then the cell mats were washed twice with PBS, and then the plates were frozen at −80° C. until miRNA extraction.

d. Total RNA Extraction

The miRNAs were extracted according to the supplier's recommendations with the miRNeasy microkit kit (Qiagen).

e. Reverse Transcription Reaction

The RNAs were reverse transcribed with the High Capacity cDNA reverse transcription kit (Applied Biosystem). 400 ng of RNA were added to 2 μL of RT buffer (10×), 2 μL of Random primer (10×), 0.8 μL of dNTPs (25×), 0.5 μL of RNAse OUT (40 U/μL) and 1 μL of multiscribe reverse transcriptase (50 U/μL) in a total volume of 20 μL. The reverse transcription reaction was carried out at 25° C. for 10 min and then 42° C. for 2 h. The activity of the enzyme was stopped by incubating the mixture at 85° C. for 5 min.

f. Quantification of miRNAs Precursors by Quantitative RT-PCR

The amount of miRNAs precursors in cells treated or not treated with miPEP was evaluated by quantitative RT-PCR with the SsoFast EvagGreen kit. For the RT-PCR reaction, 3 μL of reverse transcriptase diluted ⅕ were added to 1× of SsoFast EvaGreen, 10 μM of each primer and RNase free water in a final volume of 10 μL. The reaction was subjected to the following temperatures: 3 min pre-amplification at 95° C., 40 cycles of 10 sec at 95° C. and 30 sec at 60° C. in a thermal cycler (CFX96 Real-time PCR detection system, Biorad). PPIA was used as a reference gene to normalize quantitative RT-PCR.

For all PCR reactions, two replicates were used for each biological sample.

TABLE 8
Sequence of the primers.

| | | Forward primer (5′-3′) | Reverse primer (5′-3′) |
|---|---|---|---|
| Reference | PPIA | TCT TTG GGA CCT TGT CTG | GCC GAG GAA AAC CGT GTA |
| gene | | CAA (SEQ ID NO: 11 759) | CTA T (SEQ ID NO: 11 760) |
| miRNA | miR15a | ATAAAACCTTGGAGTAAAGT | GCACAATATGGCCTGCACC |
| | | AGCAG | (SEQ ID NO: 11 770) |
| | | (SEQ ID NO: 11 769) | |
| | miR16 | CAGCACGTAAATATTGGCGTT | TCAACCTTACTTCAGCAGCA |
| | | A (SEQ ID NO: 11 771) | C (SEQ ID NO: 11 772) |

### Example 4

### miPEPs Immunolabeling

The production of polyclonal antibodies specifically recognizing miPEP 125a, miPEP145 and miPEP15a-16-1 is made in mice or rabbits. The animals are treated by repeated injections of miPEP triggering the production of anti-miPEP antibodies.

Mesenchymal stem cells from 6 different donors are cultured for 7 days either in proliferation medium (αMEM+10% FCS), or in differentiation medium (αMEM+2% FCS+β-Glycerophosphate+Ascorbic acid+BMP4). The presence of miPEP is then looked for in the cells by immunolabelings with the polyclonal antibodies anti-miPEP125a, anti-miPEP145 and anti-miPEP 15a-16-1. The cells are incubated for 1 h to 12 h with the primary antibodies. They are then washed 3 times with 0.1% PBS BSA and then treated by adding anti-species secondary antibodies (anti-mouse or anti-rabbit) conjugated to a fluorescent probe. The reading is done under an epifluorescence microscope.

### Example 5

### In Vivo Bone Formation by Subcutaneous Implantation in Nude Mice

All experiments are done on animals in accordance with the directive 2010/63/EU and after validation of the protocols by the animal ethics committee (Toulouse). A total of 6-8 mice was used per group. The cultured cells are osteo-induced using miPEPs, and then suspended for 1 to 2 hours in the culture medium also containing biomaterial granules in the ratio of 2×10⁶ cells/50 mg of biomaterial. This biomaterial is biphasic inorganic calcium phosphate (BCP) composed of hydroxyapatite (HA) and beta-tricalcium phosphate (β-TCP) in a ratio of 20/80, ranging in size from 0.5 to 1 mm (sold by Biomatlante, Vigneux de Bretagne, France). The porosity of this biomaterial (% vol) was 75±5% of pores of which 70% from 0 to 10 μm, 20% from 10 to 100 μm and 10% from 100 to 300 μm. The biomaterial is sterilized by autoclaving.

Female Nude mice (RjOrl: NMRI-Foxnnul/Foxnlnu) (Laboratoires Janvier, Saint-Berthevin, France) are generally anesthetized by inhalationn of isoflurane. The biomaterial alone is implanted as a negative control. Two implants (cells+biomaterials) are subcutaneously placed in the back of the mice on each side of the spine (a mouse may contain different types of implants). After 8 weeks, the treated animals are sacrificed. The implants are then removed and fixed in a buffered 4% formalin solution.

### Example 6

### Pulmonary Fibrosis

miR-29 as Therapeutic Target

The therapeutic benefit of increasing the level of miR-29 has been demonstrated for fibrosis in the heart, kidney, liver, lung and in a context of systemic sclerosis. The ability of a miR-29b analog to directly target the expression of Collagen1a1 (Col1a1), whose deregulation is implicated in the mechanism of fibrosis, has been demonstrated. Thus, an increasing amount of this analog results in a dose-dependent decrease in Col1a1 expression in a cell model (Montgomery et al., MicroRNA mimicry blocks pulmonary fibrosis, EMBO Mol. Med. (2014), 6(10); 1347-1356).

Demonstration of the Therapeutic Effect of a miPEP Encoded by the Primary Transcript of miR-29

NIH 3T3 cells (mouse fibroblast cell line) are cultured on DMEM medium, supplemented with 1-Glutamine (4 mM), Sodium Pyruvate (1 mM) and a 10% BCS of BCS solution. The cells are transfected with Dharmafect I according to the reseller's recommendations and then with increasing amounts of a miPEP encoded by the primary miR-29 transcript. The cells are collected 48 hours after transfection and Col1a1 expression is measured by qPCR.

### Example 7

### Lung Cancer (Non-Small Cell Lung Cancer)

miR let-7 as a Therapeutic Target

miR letR-7 is known to act as a tumor suppressor gene in a variety of human tissues, particularly in the lung, by downregulating the post-transcriptional expression of multiple oncogenes, including RAS, MYC and HMGA2, as well as other genes for cell cycle progression. Increasing amounts of miR let-7 in overexpressing KRAS mutant cells showed a dose-dependent reduction in kras expression (Esquela-Kerscher et al., The let-7 microRNA reduces tumor growth in mouse models of lung cancer, Cell Cycle (2008), 7(6), 759-764).

Demonstration of the Therapeutic Effect of a miPEP Encoded by the Primary Transcript of miR let-7

A549 cells (KRAS overexpressing mutant cell line) were cultured on DMEM medium (90%) supplemented with fetal calf serum (10%) and 1× penicillin/streptomycin solution, then transfected with increasing amount of miPEP encoded by the primary transcript of miR let-7. The next day, the cells are rinsed and incubated for 48 hours with a conventional culture medium. The proliferation tests are carried out with the AlamarBlue kit according to the distributor's recommendations.

### Example 8

### Ischemia—Reperfusion

miR-7 as Therapeutic Target

miR-7 is implicated in the deleterious effects of ischemia-reperfusion syndrome (I/R) and is overexpressed in simulated I/R cardiomyocites. In addition, miR-7 has been shown to protect myocardial cells against apoptosis by reducing the expression of poly(ADP-ribose) polymerase (PARP). An increase in miR-7 induces a dose-dependent reduction in PARP expression (Li et al., MicroRNA-7a/b Protects against Cardiac Myocyte Injury in Ischemia/Reperfusion by Targeting Poly(ADP-Ribose) Polymerase, PLoS One (2014), 9(3); e90096).

Demonstration of the Therapeutic Effect of a miPEP Encoded by the Primary Transcript of miR-7

H9c2 cells (rat ventricular cell line) are cultured at 37° C. under 5% CO₂ on DMEM medium (90%) supplemented with fetal calf serum (10%) and 100 μg/mL penicillin/streptomycin solution. 48 hours after transfection of a miPEP encoded by the primary miR-7 transcript, the cells are subjected to a simulated I/R episode: the medium is replaced by glucose- and serum-deficient DMEM, and the cells are placed in a hypoxic chamber at 37° C. for 10 h and then reoxygenated for 2 h on DMEM medium containing 10% of fetal calf serum. Quantification of PARP expression is conducted in Western Blot with anti-PARP antibodies.

### Example 9

### Hepatitis C Virus Infection (HCV)

miR-181c as Therapeutic Target

The expression of Homeobox A1 (HOXA1) is increased in hepatocytes infected with HCV. Exogenous expression of a miR-181c analog inhibits HOXA1 and other cascading agents, including STAT3 and STATS, involved in the regulation of cell growth. The analog also suppresses HCV replication (Mukherjee et al., Transcriptional Suppression of miR-181c by Hepatitis C Virus Enhances Homeobox A1 Expression, J. Virol. (2014), 88(14); 7929-7940).

Demonstration of the Therapeutic Effect of a miPEP Encoded by the Primary Transcript of miR-181c

The human hepatoma cells (Huh7.5) are maintained on DMEM medium (90%) supplemented with fetal calf serum (10%) and antibiotics (100 U penicillin G/mL and 100 μg streptomycin/mL). HCV genotype 2a (clone JFH1) is grown in Huh7.5 cells. The supernatant is filtered on a cellulose acetate membrane. For infection, Huh7.5 cells are incubated with the viral solution for 72 h. The cells reflecting the presence of HCV genotype 2a are cultured on DMEM medium (90%) supplemented with fetal calf serum (10%) and 1% penicillin/streptomycin solution. All cells are maintained at 37° C. under 5% CO₂ and transfected with increasing doses of a miPEP encoded by the primary miR-181c transcript. The quantification of HOXA1 is carried out by Western Blot using dedicated antibodies.

### Example 10

### Evaluation of the Effects of miPEP125a on In Vivo Bone Formation by Subcutaneous Implantation in Nude Mice

### 10.1—Methods

All experiments on animals were done in accordance with directive 2010/63/EU and after validation of protocols by the Animal Ethics Committee (Toulouse, N: 86/809 EEC). A total of 6-8 mice was used per group. The cells cultured osteo-induced or not according to the methods described elsewhere (see Example 1), were then suspended for 1 to 2 h in the culture medium also containing granules of biomaterials in the ratio of 2×10⁶ cells/50 mg of biomaterial. These biomaterials were biphasic inorganic calcium phosphate (BCP), composed of hydroxyapatite (HA) and beta-tricalcium phosphate (β-TCP) in a ratio of 20/80, ranging in size from 0.5 to 1 mm (sold by Biomatlante, Vigneux de Bretagne, France). The porosity of these biomaterials (% vol) was 75±5% of pores of which 70% (0 to 10 μm), 20% (10 to 100 μm) and 10% (100 to 300 μm). They were sterilized by autoclaving.

The biomaterial alone has been implanted as a negative control. Female Nude mice (RjOrl: NMRI-Foxnnul/Foxnlnu) (Laboratoires Janvier, Saint-Berthevin, France) are generally anesthetized by inhalationn of isoflurane. Two implants (cells+biomaterials) are subcutaneously placed in the back of the mice on each side of the spine. After 4 weeks, the treated animals are sacrificed. The implants are then removed and fixed in a buffered 4% formalin solution. Some groups of mice will also receive regular injections (once a week) during the study either of the miPEP//125a-1 (SEQ ID NO: 11 752) or the control miPEP (scramble ScmiPEP//125a, SEQ ID NO: 11 758).

These implants were then decalcified in a solution of 4.13% EDTA/0.2% PFA in PBS for 96 hours at 50° C. using an automatic microwave for demineralization (KOS Histostation, Milestone Med. Corp. USA). The samples were dehydrated in ethanol and then butanol baths in an automatic dehydration apparatus (MicromMicrotech, Lyon, France). The samples were then immersed in paraphine (Histowax, Histolab, Gottenburg, Sweden). Fine sections (3 mm thick) were made using a microtome (Leica RM2255, Leica Biosystems, Nanterre, France). The sections were stained by the Masson trichrome technique and the collagen in light green. The photos were obtained by a scan of each cut (NanoZoomer, Hamamatsu, Photonics, Hamamatsu City, Shizuoka, Japan) and observed virtually (NDP view, Hamamatsu). The histomorphometry of the images was done using the ImageJ software and the neoformed bone percentage was calculated by explant area. 3 to 4 sections per explant were analyzed and quantified.

### 10.2—Materials

Female Nude mice (RjOrl: NMRI-Foxnnu1/Foxn1nu), from the approved supplier Janvier (Laboratoires Janvier, Saint-Berthevin, France),
Biomaterials: Biphasic inorganic calcium phosphate (BCP), composed of hydroxyapatite (HA) and beta-tricalcium phosphate (β-TCP) in a ratio of 20/80, ranging in size from 0.5 to 1 mm (sold by Biomatlante, Vigneux de Bretagne, France),
1.5 mL Eppendorf tubes,
Anesthesia table with Isoflurane,
Syringes 19G,
An automatic microwave for demineralization (KOS Histostation, Milestone Med. Corp. USA),
Demineralization solution of 4,13% EDTA/0.2% PFA in PBS,
Ethanol, Butanol,
Automatic dehydration device (MicromMicrotech, Lyon, France),
Microtome (Leica RM2255, Leica Biosystems, Nanterre, France),
Paraphine (Histowax, Histolab, Gottenburg, Sweden),
NanoZoomer (Hamamatsu, Photonics, Hamamatsu City, Shizuoka, Japon),
NDPview software (NDP view, Hamamatsu).

### 10.3—Results

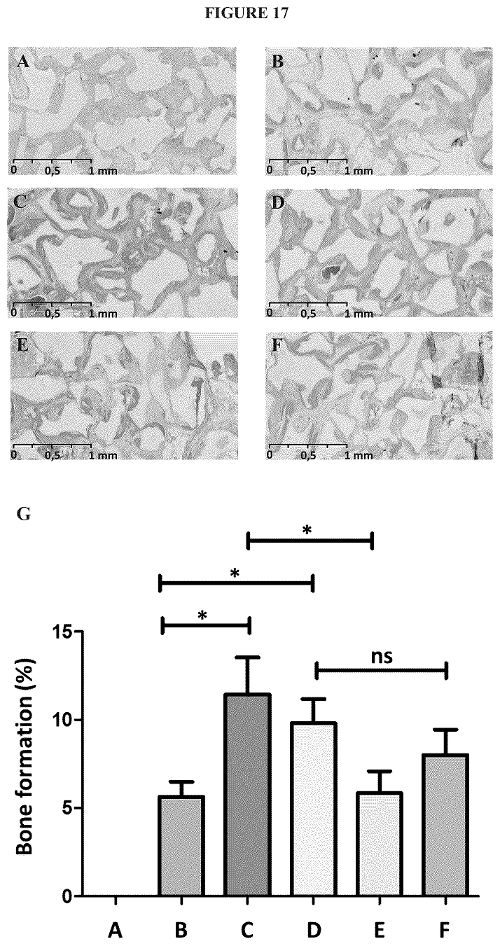

The images in FIG. 17 show that there is no bone formation in the biomaterial control alone (FIG. 17A). In contrast, the addition of MSCs to the biomaterial generated bone formation in all cases (FIG. 17B-F). However, qualitatively it appears that this bone formation is greater when the MSCs have been pre-treated with miPEP125a-1 (FIG. 17C-D) than with the scramble miPEP (FIG. 17E-F).

Quantification of neoformed bone confirms this assessment (FIG. 17G). Indeed, there is significantly more bone formation when the MSCs were pre-treated with miPEP//125a in culture (C) than in the associated control (E: MSC pre-treated with the scramble miPEP, p=0.0499). This quantification also shows that the injection of the miPEP//125a-1 in vivo (D) is without consequence, or even decreases (in a non-significant manner) the induction effect compared to its control (C: MSCs only pre-treated). On the other hand, it is clear that the bone formation of MSCs only pre-treated in vitro (C) or MSCs pre-treated in vitro and then in vivo (D) is significantly higher than the untreated MSCs (B) (p=0.0241 and p=0.0203 respectively).

The treatment of human MSCs with miPEP//125a-1 has therefore increased their ability to form bone in vivo when previously treated in vitro.

The listing of sequences of the present invention is identical to that of the priority document, which corresponds to the French application No. FR16/63245 filed on Dec. 22, 2016.

1. A method of treatment of a pathology comprising the administration of a miPEP, or a fragment of said miPEP, as a drug, said miPEP comprising from 3 to 500 amino acids encoded by a nucleotide sequence contained in the primary transcript of a miR, said miPEP being capable of modulating the accumulation of said miR in a eukaryotic cell, which miR regulates expression of at least one gene involved in a pathology, wherein said miPEP is selected from the group consisting of SEQ ID NO: 2q-1, q varying from 1 to 5,872.

2. The method of claim 1, wherein said miPEP is identified, or as identified, by implementing of a process for identifying a miPEP modulating the accumulation of a miR involved in a pathology, comprising:
a) a step of detecting an open reading frame from 12 to 1 503 nucleotides contained in the sequence of the primary transcript of said miR, then
b) a step of comparison between:
i) the accumulation of said miR in a specified eukaryotic cell expressing the primary transcript of said miR, in the presence of a peptide encoded by a nucleotide sequence that is identical or degenerate relative to that of said open reading frame, said peptide being present in the cell independently of transcription of the primary transcript of said miR, and
ii) the accumulation of said miR in a eukaryotic cell of the same type as the aforesaid specified eukaryotic cell expressing the primary transcript of said miR, in the absence of said peptide,
in which a modulation of the accumulation of said miR in the presence of said peptide relative to the accumulation of said miR in the absence of said peptide indicates the existence of a miPEP, the latter being encoded by said open reading frame and being capable of modulating the accumulation of said miR involved in a pathology.

3. The method of claim 1, wherein said miPEP is encoded by a nucleotide sequence contained in the primary transcript of a miR selected from those presented in Tables 2, 3, 4, 5 and 6, excluding the sequences SEQ ID NOs: 11,745; 11,747 and 11,749.

4. The method of claim 1, wherein said pathology is selected from the group consisting of: cancer, bacterial infections, mycoses, cardiovascular diseases, hereditary congenital diseases, skin diseases, eye diseases, diseases of the digestive system, diseases of the endocrine system, diseases of the nervous system, diseases related to viruses, diseases related to nutrition and metabolism, lymphatic diseases and hemopathies, neonatal and hereditary diseases, respiratory diseases, urogenital diseases in humans, urogenital diseases in women, disorders of the immune system, musculoskeletal disorders, and diseases or trauma of bone tissue or cartilage tissue.

5. The method of claim 1, wherein said pathology is a disease or trauma of bone tissue or cartilage tissue.

6. The method of claim 1, wherein said miPEP, or said fragment of said miPEP, is fused or linked to one or more molecules facilitating the entry of the miPEP, or miPEP fragment, into the cell.

7. The method of claim 1, wherein said miPEP, or fragment of said miPEP, is fused at the N-terminus of the C-terminus to the TAT peptide (YGRKKRRQRRR, SEQ ID NO: 11,754).

8. A process for identifying a miPEP modulating the accumulation of a miR involved in a pathology, said miPEP being encoded by a nucleotide sequence contained in the primary transcript of a miR, comprising:
a) a step of detecting an open reading frame from 12 to 1,503 nucleotides contained in the sequence of the primary transcript of said miR, then
b) a step of comparison between:
i) the accumulation of said miR in a specified eukaryotic cell expressing the primary transcript of said miR, in the presence of a peptide encoded by a nucleotide sequence that is identical or degenerate relative to that of said open reading frame, said peptide being present in the cell independently of transcription of the primary transcript of said miR, and
ii) the accumulation of said miR in a eukaryotic cell of the same type as the aforesaid specified eukaryotic cell expressing the primary transcript of said miR, in the absence of said peptide, in which a modulation of the accumulation of said miR in the presence of said peptide
in which a modulation of the accumulation of said miR in the presence of said peptide relative to the accumulation of said miR in the absence of said peptide indicates the existence of a miPEP, the latter being encoded by said open reading frame and being capable of modulating the accumulation of said miR involved in a pathology.

9. A pharmaceutical composition comprising:
1) a miPEP from 3 to 500 amino acids encoded by a nucleotide sequence contained in the primary transcript of a miR, or a fragment of said miPEP, said miPEP being capable of modulating the accumulation of said miR in a eukaryotic cell, wherein said miPEP is selected from the group consisting of SEQ ID NO: 2q-1, q varying from 1 to 5,872; or
2) a nucleic acid encoding said miPEP, or a fragment of said miPEP, said miPEP being capable of modulating the accumulation of said miR in a eukaryotic cell, wherein said nucleic acid is selected from the group consisting of SEQ ID NO: 2q, q varying from 1 to 5,872, and
a pharmaceutically acceptable excipient.

10. The pharmaceutical composition of claim 9, wherein said miPEP is identified, or as identified, by implementing of a process for identifying a miPEP modulating the accumulation of a miR involved in a pathology, comprising:
a) a step of detecting an open reading frame from 12 to 1 503 nucleotides contained in the sequence of the primary transcript of said miR, then
b) a step of comparison between:
i) the accumulation of said miR in a specified eukaryotic cell expressing the primary transcript of said miR, in the presence of a peptide encoded by a nucleotide sequence that is identical or degenerate relative to that of said open reading frame, said peptide being present in the cell independently of transcription of the primary transcript of said miR, and
ii) the accumulation of said miR in a eukaryotic cell of the same type as the aforesaid specified eukaryotic cell expressing the primary transcript of said miR, in the absence of said peptide,
in which a modulation of the accumulation of said miR in the presence of said peptide relative to the accumulation of said miR in the absence of said peptide indicates the existence of a miPEP, the latter being encoded by said open reading frame and being capable of modulating the accumulation of said miR involved in a pathology.

11. The pharmaceutical composition of claim 9, wherein said miPEP is selected from the group of miPEP consisting of SEQ ID NO: 2q-1, q varying from 1 to 5,872.

12. The pharmaceutical composition of claim 9, wherein said miPEP, or said fragment of said miPEP, is fused or linked to one or more molecules facilitating the entry of the miPEP, or miPEP fragment, into the cell.

13. The pharmaceutical composition of claim 9, wherein said miPEP, or fragment of said miPEP, is fused at the N-terminus or the C-terminus to the TAT peptide
(YGRKKRRQRRR, SEQ ID NO: 11, 754).

14. The pharmaceutical composition of claim 9, wherein said nucleic acid is selected from the group consisting of: SEQ ID NO: 2q, q varying from 1 to 5,872.
